(19) United States
(12) Patent Application Publication
(10) Pub. No.: US 2022/0389394 A1
(43) Pub. Date: Dec. 8, 2022

(54) METHODS OF USING FLT3L-Fc FUSION PROTEINS

(71) Applicant: Gilead Sciences, Inc., Foster City, CA (US)

(72) Inventors: Anees M. Dauki, Foster City, CA (US); Michelle R. Kuhne, San Francisco, CA (US); Ahmed A. Othman, Dublin, CA (US); Nishanthan Rajakumaraswamy, San Ramon, CA (US)

(21) Appl. No.: 17/744,515

(22) Filed: May 13, 2022

Related U.S. Application Data
(60) Provisional application No. 63/190,168, filed on May 18, 2021.

(51) Int. Cl. C12N 9/12; A61P 35/02; A61K 38/45; A61K 45/06; A61K 47/68

(52) U.S. Cl. CPC C12N 9/12; A61P 35/02; C12Y 207/10001; A61K 38/45; A61K 45/06; A61K 47/6803; C07K 2319/30

(57) ABSTRACT

Provided methods of using FLT3L-Fc fusion proteins, including doses and dosing regimens and schedules for administering FLT3L-Fc fusion proteins to a subject in need thereof.

### CROSS REFERENCE TO RELATED APPLICATIONS

This application claims the benefit of U.S. provisional application Ser. No. 63/190,168 filed on May 18, 2021. The entire content of this application is incorporated herein by reference in its entirety.

### SEQUENCE LISTING

The instant application contains a Sequence Listing which has been submitted electronically in ASCII format and is hereby incorporated by reference in its entirety. Said ASCII copy, created on May 11, 2021, is named 1376-US-NP_SL.txt and is 210,078 bytes in size.

### BACKGROUND

Dendritic cells (DCs) are the most potent antigen-presenting cell in the body. DCs function to process antigen material and present it on the cell surface to the T cells. DCs act as messengers between the innate and the adaptive immune systems. Fms related tyrosine kinase 3 ligand (FLT3LG, FLT3L, NCBI Gene ID: 2323) selectively expands DCs from bone marrow precursors, as well as promotes proliferation of terminally differentiated DCs in lymphoid and tumor tissues.

Soluble recombinant human protein forms of FLT3L have a serum half-life in humans of about 12-28 hours after five consecutive subcutaneous (SC) doses, requiring daily administration to the patient over a 28-day therapeutic cycle. Daily administration is undesirable, for both the patient and clinician, and is dose scheduling that does not align with other approved immune-oncology therapeutic agents, which is usually once every 2 to 3 weeks. Disclosed herein methods of administering to a subject a fusion protein comprising a human fms related tyrosine kinase 3 ligand (FLT3L) extracellular domain operably linked to an immunoglobulin fragment crystallizable region (Fc region). The methods disclosed herein provide safe and effective doses and dosing regimens and schedules for administering the fusion protein to a subject in need thereof.

### SUMMARY

Provided herein are methods of preventing, reducing and/or inhibiting the recurrence, growth, proliferation, migration and/or metastasis of a cancer cell or population of cancer cells in a subject in need thereof. In some embodiments, the method comprises administering to the subject at least about 200 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 225 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 675 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 200 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart, and wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month, and wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject two or more doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the two or more doses are administered at least 2 weeks apart over a duration of at least 1 month, and wherein (a) at least 5 amino acids are truncated from the C-terminus of the FLT3L extracellular domain; and/or (b) the Fc region does not comprise a hinge region. In some embodiments, the method comprises administering to the subject two or more doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the two or more doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and (B) administering one or more subsequent doses of an effective amount of the fusion protein to the subject, wherein the dosing interval between the last dose of step A and the first dose of step B is greater than the dosing interval for the two or more doses of step A, and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of a fusion protein comprising a human fms related tyrosine kinase 3 ligand (FLT3L) extracellular domain operably linked to an immunoglobulin fragment crystallizable region (Fc region), wherein (i) each dose comprises between about 200 μg to about 30000 μg of the fusion protein; and (ii) the dosing interval for the two or more doses is once every 2 to 4 weeks; and (B) administering one or more subsequent doses of an effective amount of the fusion protein to the subject, wherein the dosing interval between the last dose of step A and the first dose of step B is greater than the dosing interval for the two or more doses of step A; and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and (B) administering one or more subsequent doses of an effective amount of the fusion protein to the subject, wherein the dosing interval between the last dose of step A and the first dose of step B is between about 6 weeks to about 8 months; and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the two or more doses are administered about 8 to 20 days apart; (B) administering to the subject two or more subsequent doses of an effective amount of the fusion protein, wherein the dosing interval for the two or more subseqEuent doses is between about 21 to 36 days apart; (C) pausing administration of the fusion protein to the subject for a period of between about 6 weeks to about 8 months; and (D) repeating the administration of any one of steps A and B, wherein (a) at least 5 amino acids are truncated from the C-terminus of the FLT3L extracellular domain; and/or (b) the Fc region does not comprise a hinge region. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27. In some embodiments, the fusion protein comprises an amino acid sequence selected from the group consisting of SEQ ID NOs: 1-18 and 21-27. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 14. In some embodiments, the fusion protein comprises the amino acid sequence of SEQ ID NO: 14. In some embodiments, the method further comprises co-administering to the subject an anti-cancer agent. In some embodiments, the anti-cancer agent is sacituzumab govitecan. In some embodiments, the anti-cancer agent is magrolimab. In some embodiments, the anti-cancer agent is an anti-CD47 antibody. In some embodiments, the anti-cancer agent is an inhibitor of MCL-1. In some embodiments, the method further comprises co-administering to the subject an immunotherapy. In some embodiments, the method further comprises co-administering to the subject an immune checkpoint protein or receptor. In some embodiments, the method further comprises co-administering to the subject an inhibitor of an immune checkpoint protein or receptor. In some embodiments, the method further comprises co-administering to the subject an activator of an immune checkpoint protein or receptor.

Provided herein are methods of treating or inhibiting cancer in a subject in need thereof. In some embodiments, the method comprises administering to the subject at least about 200 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 225 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 675 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 200 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart, and wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month, and wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject two or more doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the two or more doses are administered at least 2 weeks apart over a duration of at least 1 month, and wherein (a) at least 5 amino acids are truncated from the C-terminus of the FLT3L extracellular domain; and/or (b) the Fc region does not comprise a hinge region. In some embodiments, the method comprises administering to the subject two or more doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the two or more doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and (B) administering one or more subsequent doses of an effective amount of the fusion protein to the subject, wherein the dosing interval between the last dose of step A and the first dose of step B is greater than the dosing interval for the two or more doses of step A, and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of a fusion protein comprising a human fms related tyrosine kinase 3 ligand (FLT3L) extracellular domain operably linked to an immunoglobulin fragment crystallizable region (Fc region), wherein (i) each dose comprises between about 200 μg to about 30000 μg of the fusion protein; and (ii) the dosing interval for the two or more doses is once every 2 to 4 weeks; and (B) administering one or more subsequent doses of an effective amount of the fusion protein to the subject, wherein the dosing interval between the last dose of step A and the first dose of step B is greater than the dosing interval for the two or more doses of step A; and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and (B) administering one or more subsequent doses of an effective amount of the fusion protein to the subject, wherein the dosing interval between the last dose of step A and the first dose of step B is between about 6 weeks to about 8 months; and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the two or more doses are administered about 8 to 20 days apart; (B) administering to the subject two or more subsequent doses of an effective amount of the fusion protein, wherein the dosing interval for the two or more subsequent doses is between about 21 to 36 days apart; (C) pausing administration of the fusion protein to the subject for a period of between about 6 weeks to about 8 months; and (D) repeating the administration of any one of steps A and B, wherein (a) at least 5 amino acids are truncated from the C-terminus of the FLT3L extracellular domain; and/or (b) the Fc region does not comprise a hinge region. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27. In some embodiments, the fusion protein comprises an amino acid sequence selected from the group consisting of SEQ ID NOs: 1-18 and 21-27. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 14. In some embodiments, the fusion protein comprises the amino acid sequence of SEQ ID NO: 14. In some embodiments, the method further comprises co-administering to the subject an anti-cancer agent. In some embodiments, the anti-cancer agent is sacituzumab govitecan. In some embodiments, the anti-cancer agent is magrolimab. In some embodiments, the anti-cancer agent is an anti-CD47 antibody. In some embodiments, the anti-cancer agent is an inhibitor of MCL-1. In some embodiments, the method further comprises co-administering to the subject an immunotherapy. In some embodiments, the method further comprises co-administering to the subject an immune checkpoint protein or receptor. In some embodiments, the method further comprises co-administering to the subject an inhibitor of an immune checkpoint protein or receptor. In some embodiments, the method further comprises co-administering to the subject an activator of an immune checkpoint protein or receptor.

Provided herein are methods of enhancing, promoting, and/or increasing the tumor infiltration of T-cells and/or NK cells in a subject in need thereof. In some embodiments, the method comprises administering to the subject at least about 200 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 225 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 675 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 200 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart, and wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month, and wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject two or more doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the two or more doses are administered at least 2 weeks apart over a duration of at least 1 month, and wherein (a) at least 5 amino acids are truncated from the C-terminus of the FLT3L extracellular domain; and/or (b) the Fc region does not comprise a hinge region. In some embodiments, the method comprises administering to the subject two or more doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the two or more doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and (B) administering one or more subsequent doses of an effective amount of the fusion protein to the subject, wherein the dosing interval between the last dose of step A and the first dose of step B is greater than the dosing interval for the two or more doses of step A, and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of a fusion protein comprising a human fms related tyrosine kinase 3 ligand (FLT3L) extracellular domain operably linked to an immunoglobulin fragment crystallizable region (Fc region), wherein (i) each dose comprises between about 200 μg to about 30000 μg of the fusion protein; and (ii) the dosing interval for the two or more doses is once every 2 to 4 weeks; and (B) administering one or more subsequent doses of an effective amount of the fusion protein to the subject, wherein the dosing interval between the last dose of step A and the first dose of step B is greater than the dosing interval for the two or more doses of step A; and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and (B) administering one or more subsequent doses of an effective amount of the fusion protein to the subject, wherein the dosing interval between the last dose of step A and the first dose of step B is between about 6 weeks to about 8 months; and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the two or more doses are administered about 8 to 20 days apart; (B) administering to the subject two or more subsequent doses of an effective amount of the fusion protein, wherein the dosing interval for the two or more subsequent doses is between about 21 to 36 days apart; (C) pausing administration of the fusion protein to the subject for a period of between about 6 weeks to about 8 months; and (D) repeating the administration of any one of steps A and B, wherein (a) at least 5 amino acids are truncated from the C-terminus of the FLT3L extracellular domain; and/or (b) the Fc region does not comprise a hinge region. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27. In some embodiments, the fusion protein comprises an amino acid sequence selected from the group consisting of SEQ ID NOs: 1-18 and 21-27. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 14. In some embodiments, the fusion protein comprises the amino acid sequence of SEQ ID NO: 14. In some embodiments, the method further comprises co-administering to the subject an anti-cancer agent. In some embodiments, the anti-cancer agent is sacituzumab govitecan. In some embodiments, the anti-cancer agent is magrolimab. In some embodiments, the anti-cancer agent is an anti-CD47 antibody. In some embodiments, the anti-cancer agent is an inhibitor of MCL-1. In some embodiments, the method further comprises co-administering to the subject an immunotherapy. In some embodiments, the method further comprises co-administering to the subject an immune checkpoint protein or receptor. In some embodiments, the method further comprises co-administering to the subject an inhibitor of an immune checkpoint protein or receptor. In some embodiments, the method further comprises co-administering to the subject an activator of an immune checkpoint protein or receptor.

Provided herein are methods of enhancing, promoting, and/or accelerating the recovery from or reversing the effects of lymphopenia in a subject in need thereof. In some embodiments, the method comprises administering to the subject at least about 200 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 225 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 675 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 200 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart, and wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month, and wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject two or more doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the two or more doses are administered at least 2 weeks apart over a duration of at least 1 month, and wherein (a) at least 5 amino acids are truncated from the C-terminus of the FLT3L extracellular domain; and/or (b) the Fc region does not comprise a hinge region. In some embodiments, the method comprises administering to the subject two or more doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the two or more doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and (B) administering one or more subsequent doses of an effective amount of the fusion protein to the subject, wherein the dosing interval between the last dose of step A and the first dose of step B is greater than the dosing interval for the two or more doses of step A, and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of a fusion protein comprising a human fms related tyrosine kinase 3 ligand (FLT3L) extracellular domain operably linked to an immunoglobulin fragment crystallizable region (Fc region), wherein (i) each dose comprises between about 200 μg to about 30000 μg of the fusion protein; and (ii) the dosing interval for the two or more doses is once every 2 to 4 weeks; and (B) administering one or more subsequent doses of an effective amount of the fusion protein to the subject, wherein the dosing interval between the last dose of step A and the first dose of step B is greater than the dosing interval for the two or more doses of step A; and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and (B) administering one or more subsequent doses of an effective amount of the fusion protein to the subject, wherein the dosing interval between the last dose of step A and the first dose of step B is between about 6 weeks to about 8 months; and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the two or more doses are administered about 8 to 20 days apart; (B) administering to the subject two or more subsequent doses of an effective amount of the fusion protein, wherein the dosing interval for the two or more subsequent doses is between about 21 to 36 days apart; (C) pausing administration of the fusion protein to the subject for a period of between about 6 weeks to about 8 months; and (D) repeating the administration of any one of steps A and B, wherein (a) at least 5 amino acids are truncated from the C-terminus of the FLT3L extracellular domain; and/or (b) the Fc region does not comprise a hinge region. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27. In some embodiments, the fusion protein comprises an amino acid sequence selected from the group consisting of SEQ ID NOs: 1-18 and 21-27. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 14. In some embodiments, the fusion protein comprises the amino acid sequence of SEQ ID NO: 14. In some embodiments, the method further comprises co-administering to the subject an anti-cancer agent. In some embodiments, the anti-cancer agent is sacituzumab govitecan. In some embodiments, the anti-cancer agent is magrolimab. In some embodiments, the anti-cancer agent is an anti-CD47 antibody. In some embodiments, the anti-cancer agent is an inhibitor of MCL-1. In some embodiments, the method further comprises co-administering to the subject an immunotherapy. In some embodiments, the method further comprises co-administering to the subject an immune checkpoint protein or receptor. In some embodiments, the method further comprises co-administering to the subject an inhibitor of an immune checkpoint protein or receptor. In some embodiments, the method further comprises co-administering to the subject an activator of an immune checkpoint protein or receptor.

Provided herein are methods of enhancing, improving, and/or increasing the response to an anticancer therapy in a subject in need thereof. In some embodiments, the method comprises co-administering to the subject (I) at least about 200 μg of a fusion protein comprising a human fms related tyrosine kinase 3 ligand (FLT3L) extracellular domain operably linked to an immunoglobulin fragment crystallizable region (Fc region); and (II) an anti-cancer agent, wherein (a) at least 5 amino acids are truncated from the C-terminus of the FLT3L extracellular domain; and/or (b) the Fc region does not comprise a hinge region. In some embodiments, the method comprises co-administering to the subject (I) at least about 225 μg of a fusion protein comprising a human fms related tyrosine kinase 3 ligand (FLT3L) extracellular domain operably linked to an immunoglobulin fragment crystallizable region (Fc region); and (II) an anti-cancer agent, wherein (a) at least 5 amino acids are truncated from the C-terminus of the FLT3L extracellular domain; and/or (b) the Fc region does not comprise a hinge region. In some embodiments, the method comprises co-administering to the subject (I) at least about 675 μg of a fusion protein comprising a human fms related tyrosine kinase 3 ligand (FLT3L) extracellular domain operably linked to an immunoglobulin fragment crystallizable region (Fc region); and (II) an anti-cancer agent, wherein (a) at least 5 amino acids are truncated from the C-terminus of the FLT3L extracellular domain; and/or (b) the Fc region does not comprise a hinge region. In some embodiments, the method comprises co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region); and (II) an anti-cancer agent, wherein (a) at least 5 amino acids are truncated from the C-terminus of the FLT3L extracellular domain; and/or (b) the Fc region does not comprise a hinge region. In some embodiments, the method comprises co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart; and (II) an anti-cancer agent, wherein (a) at least 5 amino acids are truncated from the C-terminus of the FLT3L extracellular domain; and/or (b) the Fc region does not comprise a hinge region. In some embodiments, the method comprises co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart; and (II) an anti-cancer agent, and wherein (a) at least 5 amino acids are truncated from the C-terminus of the FLT3L extracellular domain; and/or (b) the Fc region does not comprise a hinge region. In some embodiments, the method comprises co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month; and (II) an anti-cancer agent, and wherein (a) at least 5 amino acids are truncated from the C-terminus of the FLT3L extracellular domain; and/or (b) the Fc region does not comprise a hinge region. In some embodiments, the method comprises co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months; and (II) an anti-cancer agent, and wherein (a) at least 5 amino acids are truncated from the C-terminus of the FLT3L extracellular domain; and/or (b) the Fc region does not comprise a hinge region. In some embodiments, the method comprises co-administering to the subject (I) between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months; and (II) an anti-cancer agent, and wherein (a) at least 5 amino acids are truncated from the C-terminus of the FLT3L extracellular domain; and/or (b) the Fc region does not comprise a hinge region. In some embodiments, the method comprises co-administering to the subject (I) two or more doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the two or more doses are administered at least 2 weeks apart over a duration of at least 1 month; and (II) an anti-cancer agent, and wherein (a) at least 5 amino acids are truncated from the C-terminus of the FLT3L extracellular domain; and/or (b) the Fc region does not comprise a hinge region. In some embodiments, the method comprises co-administering to the subject (I) two or more doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the two or more doses are administered between about 2 to 5 weeks apart over a duration of between about 1 to 4 months; and (II) an anti-cancer agent, and wherein (a) at least 5 amino acids are truncated from the C-terminus of the FLT3L extracellular domain; and/or (b) the Fc region does not comprise a hinge region. In some embodiments, the method comprises co-administering to the subject (I) between about 3 to 8 doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the doses are administered between about 2 to 5 weeks apart over a duration of between about 1 to 4 months; and (II) an anti-cancer agent, and wherein (a) at least 5 amino acids are truncated from the C-terminus of the FLT3L extracellular domain; and/or (b) the Fc region does not comprise a hinge region. In some embodiments, the method comprises (A) co-administering to the subject (I) two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and (II) an anti-cancer agent; and (B) administering one or more subsequent doses of an effective amount of the fusion protein to the subject, wherein the dosing interval between the last dose of step A and the first dose of step B is greater than the dosing interval for the two or more doses of step A, and wherein (a) at least 5 amino acids are truncated from the C-terminus of the FLT3L extracellular domain; and/or (b) the Fc region does not comprise a hinge region. In some embodiments, the method comprises (A) co-administering to the subject (I) two or more doses of a fusion protein comprising a human fms related tyrosine kinase 3 ligand (FLT3L) extracellular domain operably linked to an immunoglobulin fragment crystallizable region (Fc region), wherein (i) each dose comprises between about 200 μg to about 30000 μg of the fusion protein; and (ii) the dosing interval for the two or more doses is once every 2 to 4 weeks; and (II) an anti-cancer agent; and (B) administering one or more subsequent doses of an effective amount of the fusion protein to the subject, wherein the dosing interval between the last dose of step A and the first dose of step B is greater than the dosing interval for the two or more doses of step A; and wherein (a) at least 5 amino acids are truncated from the C-terminus of the FLT3L extracellular domain; and/or (b) the Fc region does not comprise a hinge region. In some embodiments, the method comprises (A) co-administering to the subject (I) two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and (II) an anti-cancer agent; and (B) administering one or more subsequent doses of an effective amount of the fusion protein to the subject, wherein the dosing interval between the last dose of step A and the first dose of step B is between about 6 weeks to about 8 months; and wherein (a) at least 5 amino acids are truncated from the C-terminus of the FLT3L extracellular domain; and/or (b) the Fc region does not comprise a hinge region. In some embodiments, the method comprises (A) co-administering to the subject (I) two or more doses of an effective amount a fusion protein comprising a human fms related tyrosine kinase 3 ligand (FLT3L) extracellular domain operably linked to an immunoglobulin fragment crystallizable region (Fc region), wherein the two or more doses are administered about 8 to 20 days apart; and (II) an anti-cancer agent; (B) administering to the subject two or more subsequent doses of an effective amount of the fusion protein, wherein the dosing interval for the two or more subsequent doses is between about 21 to 36 days apart; (C) pausing administration of the fusion protein to the subject for a period of between about 6 weeks to about 8 months; and (D) repeating the administration of any one of steps A and B, wherein (a) at least 5 amino acids are truncated from the C-terminus of the FLT3L extracellular domain; and/or (b) the Fc region does not comprise a hinge region. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27. In some embodiments, the fusion protein comprises an amino acid sequence selected from the group consisting of SEQ ID NOs: 1-18 and 21-27. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 14. In some embodiments, the fusion protein comprises the amino acid sequence of SEQ ID NO: 14. In some embodiments, the anti-cancer agent is sacituzumab govitecan. In some embodiments, the anti-cancer agent is magrolimab. In some embodiments, the anti-cancer agent is an anti-CD47 antibody. In some embodiments, the anti-cancer agent is an inhibitor of MCL-1. In some embodiments, the method further comprises co-administering to the subject an immunotherapy. In some embodiments, the method further comprises co-administering to the subject an immune checkpoint protein or receptor. In some embodiments, the method further comprises co-administering to the subject an inhibitor of an immune checkpoint protein or receptor. In some embodiments, the method further comprises co-administering to the subject an activator of an immune checkpoint protein or receptor.

Provided herein are methods of promoting, inducing and/or increasing the expansion and/or proliferation of a cell or a population of cells that express fms related tyrosine kinase 3 (FLT3, CD135) in a subject in need thereof. In some embodiments, the method comprises administering to the subject at least about 200 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 225 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 675 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 200 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart, and wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month, and wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject two or more doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the two or more doses are administered at least 2 weeks apart over a duration of at least 1 month, and wherein (a) at least 5 amino acids are truncated from the C-terminus of the FLT3L extracellular domain; and/or (b) the Fc region does not comprise a hinge region. In some embodiments, the method comprises administering to the subject two or more doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the two or more doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and (B) administering one or more subsequent doses of an effective amount of the fusion protein to the subject, wherein the dosing interval between the last dose of step A and the first dose of step B is greater than the dosing interval for the two or more doses of step A, and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of a fusion protein comprising a human fms related tyrosine kinase 3 ligand (FLT3L) extracellular domain operably linked to an immunoglobulin fragment crystallizable region (Fc region), wherein (i) each dose comprises between about 200 μg to about 30000 μg of the fusion protein; and (ii) the dosing interval for the two or more doses is once every 2 to 4 weeks; and (B) administering one or more subsequent doses of an effective amount of the fusion protein to the subject, wherein the dosing interval between the last dose of step A and the first dose of step B is greater than the dosing interval for the two or more doses of step A; and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and (B) administering one or more subsequent doses of an effective amount of the fusion protein to the subject, wherein the dosing interval between the last dose of step A and the first dose of step B is between about 6 weeks to about 8 months; and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the two or more doses are administered about 8 to 20 days apart; (B) administering to the subject two or more subsequent doses of an effective amount of the fusion protein, wherein the dosing interval for the two or more subsequent doses is between about 21 to 36 days apart; (C) pausing administration of the fusion protein to the subject for a period of between about 6 weeks to about 8 months; and (D) repeating the administration of any one of steps A and B, wherein (a) at least 5 amino acids are truncated from the C-terminus of the FLT3L extracellular domain; and/or (b) the Fc region does not comprise a hinge region. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27. In some embodiments, the fusion protein comprises an amino acid sequence selected from the group consisting of SEQ ID NOs: 1-18 and 21-27. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 14. In some embodiments, the fusion protein comprises the amino acid sequence of SEQ ID NO: 14. In some embodiments, the method further comprises co-administering to the subject an anti-cancer agent. In some embodiments, the anti-cancer agent is sacituzumab govitecan. In some embodiments, the anti-cancer agent is magrolimab. In some embodiments, the anti-cancer agent is an anti-CD47 antibody. In some embodiments, the anti-cancer agent is an inhibitor of MCL-1. In some embodiments, the method further comprises co-administering to the subject an immunotherapy. In some embodiments, the method further comprises co-administering to the subject an immune checkpoint protein or receptor. In some embodiments, the method further comprises co-administering to the subject an inhibitor of an immune checkpoint protein or receptor. In some embodiments, the method further comprises co-administering to the subject an activator of an immune checkpoint protein or receptor.

Provided herein are methods of enhancing, improving, and/or increasing the response to an immunotherapy in a subject in need thereof. In some embodiments, the method comprises co-administering to the subject (I) at least about 200 μg of a fusion protein comprising a human fms related tyrosine kinase 3 ligand (FLT3L) extracellular domain operably linked to an immunoglobulin fragment crystallizable region (Fc region); and (II) an immunotherapy, wherein (a) at least 5 amino acids are truncated from the C-terminus of the FLT3L extracellular domain; and/or (b) the Fc region does not comprise a hinge region. In some embodiments, the method comprises co-administering to the subject (I) at least about 225 μg of a fusion protein comprising a human fms related tyrosine kinase 3 ligand (FLT3L) extracellular domain operably linked to an immunoglobulin fragment crystallizable region (Fc region); and (II) an immunotherapy, wherein (a) at least 5 amino acids are truncated from the C-terminus of the FLT3L extracellular domain; and/or (b) the Fc region does not comprise a hinge region. In some embodiments, the method comprises co-administering to the subject (I) at least about 675 μg of a fusion protein comprising a human fms related tyrosine kinase 3 ligand (FLT3L) extracellular domain operably linked to an immunoglobulin fragment crystallizable region (Fc region); and (II) an immunotherapy, wherein (a) at least 5 amino acids are truncated from the C-terminus of the FLT3L extracellular domain; and/or (b) the Fc region does not comprise a hinge region. In some embodiments, the method comprises co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region); and (II) an immunotherapy, wherein (a) at least 5 amino acids are truncated from the C-terminus of the FLT3L extracellular domain; and/or (b) the Fc region does not comprise a hinge region. In some embodiments, the method comprises co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart; and (II) an immunotherapy, wherein (a) at least 5 amino acids are truncated from the C-terminus of the FLT3L extracellular domain; and/or (b) the Fc region does not comprise a hinge region. In some embodiments, the method comprises co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart; and (II) an immunotherapy, and wherein (a) at least 5 amino acids are truncated from the C-terminus of the FLT3L extracellular domain; and/or (b) the Fc region does not comprise a hinge region. In some embodiments, the method comprises co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month; and (II) an immunotherapy, and wherein (a) at least 5 amino acids are truncated from the C-terminus of the FLT3L extracellular domain; and/or (b) the Fc region does not comprise a hinge region. In some embodiments, the method comprises co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months; and (II) an immunotherapy, and wherein (a) at least 5 amino acids are truncated from the C-terminus of the FLT3L extracellular domain; and/or (b) the Fc region does not comprise a hinge region. In some embodiments, the method comprises co-administering to the subject (I) between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months; and (II) an immunotherapy, and wherein (a) at least 5 amino acids are truncated from the C-terminus of the FLT3L extracellular domain; and/or (b) the Fc region does not comprise a hinge region. In some embodiments, the method comprises co-administering to the subject (I) two or more doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the two or more doses are administered at least 2 weeks apart over a duration of at least 1 month; and (II) an immunotherapy, and wherein (a) at least 5 amino acids are truncated from the C-terminus of the FLT3L extracellular domain; and/or (b) the Fc region does not comprise a hinge region. In some embodiments, the method comprises co-administering to the subject (I) two or more doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the two or more doses are administered between about 2 to 5 weeks apart over a duration of between about 1 to 4 months; and (II) an immunotherapy, and wherein (a) at least 5 amino acids are truncated from the C-terminus of the FLT3L extracellular domain; and/or (b) the Fc region does not comprise a hinge region. In some embodiments, the method comprises co-administering to the subject (I) between about 3 to 8 doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the doses are administered between about 2 to 5 weeks apart over a duration of between about 1 to 4 months; and (II) an immunotherapy, and wherein (a) at least 5 amino acids are truncated from the C-terminus of the FLT3L extracellular domain; and/or (b) the Fc region does not comprise a hinge region. In some embodiments, the method comprises (A) co-administering to the subject (I) two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and (II) an immunotherapy; and (B) administering one or more subsequent doses of an effective amount of the fusion protein to the subject, wherein the dosing interval between the last dose of step A and the first dose of step B is greater than the dosing interval for the two or more doses of step A, and wherein (a) at least 5 amino acids are truncated from the C-terminus of the FLT3L extracellular domain; and/or (b) the Fc region does not comprise a hinge region. In some embodiments, the method comprises (A) co-administering to the subject (I) two or more doses of a fusion protein comprising a human fms related tyrosine kinase 3 ligand (FLT3L) extracellular domain operably linked to an immunoglobulin fragment crystallizable region (Fc region), wherein (i) each dose comprises between about 200 μg to about 30000 μg of the fusion protein; and (ii) the dosing interval for the two or more doses is once every 2 to 4 weeks; and (II) an immunotherapy; and (B) administering one or more subsequent doses of an effective amount of the fusion protein to the subject, wherein the dosing interval between the last dose of step A and the first dose of step B is greater than the dosing interval for the two or more doses of step A; and wherein (a) at least 5 amino acids are truncated from the C-terminus of the FLT3L extracellular domain; and/or (b) the Fc region does not comprise a hinge region. In some embodiments, the method comprises (A) co-administering to the subject (I) two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and (II) an immunotherapy; and (B) administering one or more subsequent doses of an effective amount of the fusion protein to the subject, wherein the dosing interval between the last dose of step A and the first dose of step B is between about 6 weeks to about 8 months; and wherein (a) at least 5 amino acids are truncated from the C-terminus of the FLT3L extracellular domain; and/or (b) the Fc region does not comprise a hinge region. In some embodiments, the method comprises (A) co-administering to the subject (I) two or more doses of an effective amount a fusion protein comprising a human fms related tyrosine kinase 3 ligand (FLT3L) extracellular domain operably linked to an immunoglobulin fragment crystallizable region (Fc region), wherein the two or more doses are administered about 8 to 20 days apart; and (II) an immunotherapy; (B) administering to the subject two or more subsequent doses of an effective amount of the fusion protein, wherein the dosing interval for the two or more subsequent doses is between about 21 to 36 days apart; (C) pausing administration of the fusion protein to the subject for a period of between about 6 weeks to about 8 months; and (D) repeating the administration of any one of steps A and B, wherein (a) at least 5 amino acids are truncated from the C-terminus of the FLT3L extracellular domain; and/or (b) the Fc region does not comprise a hinge region. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27. In some embodiments, the fusion protein comprises an amino acid sequence selected from the group consisting of SEQ ID NOs: 1-18 and 21-27. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 14. In some embodiments, the fusion protein comprises the amino acid sequence of SEQ ID NO: 14. In some embodiments, the method further comprises co-administering to the subject an anti-cancer agent. In some embodiments, the anti-cancer agent is sacituzumab govitecan. In some embodiments, the anti-cancer agent is magrolimab. In some embodiments, the anti-cancer agent is an anti-CD47 antibody. In some embodiments, the anti-cancer agent is an inhibitor of MCL-1. In some embodiments, the method further comprises co-administering to the subject an immune checkpoint protein or receptor. In some embodiments, the method further comprises co-administering to the subject an inhibitor of an immune checkpoint protein or receptor. In some embodiments, the method further comprises co-administering to the subject an activator of an immune checkpoint protein or receptor.

Provided herein are methods inducing the immune system in a subject in need thereof. In some embodiments, the method comprises administering to the subject at least about 200 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 225 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 675 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject at least about 200 μg of a fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart, and wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month, and wherein (a) at least 5 amino acids are truncated from the C-terminus of the FLT3L extracellular domain; and/or (b) the Fc region does not comprise a hinge region. In some embodiments, the method comprises administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject two or more doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the two or more doses are administered at least 2 weeks apart over a duration of at least 1 month, and wherein (a) at least 5 amino acids are truncated from the C-terminus of the FLT3L extracellular domain; and/or (b) the Fc region does not comprise a hinge region. In some embodiments, the method comprises administering to the subject two or more doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the two or more doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein (a) each dose comprises between about 200 μg to about 30000 μg of the fusion protein; and (b) at least two of the doses are administered between about 2 to 5 weeks apart over a duration of between about 1 to 4 months, and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and (B) administering one or more subsequent doses of an effective amount of the fusion protein to the subject, wherein the dosing interval between the last dose of step A and the first dose of step B is greater than the dosing interval for the two or more doses of step A, and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of a fusion protein comprising a human fms related tyrosine kinase 3 ligand (FLT3L) extracellular domain operably linked to an immunoglobulin fragment crystallizable region (Fc region), wherein (i) each dose comprises between about 200 μg to about 30000 μg of the fusion protein; and (ii) the dosing interval for the two or more doses is once every 2 to 4 weeks; and (B) administering one or more subsequent doses of an effective amount of the fusion protein to the subject, wherein the dosing interval between the last dose of step A and the first dose of step B is greater than the dosing interval for the two or more doses of step A; and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and (B) administering one or more subsequent doses of an effective amount of the fusion protein to the subject, wherein the dosing interval between the last dose of step A and the first dose of step B is between about 6 weeks to about 8 months; and wherein (a) at least 5 amino acids are truncated from the C-terminus of the FLT3L extracellular domain; and/or (b) the Fc region does not comprise a hinge region. In some embodiments, the method comprises (A) administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the two or more doses are administered about 8 to 20 days apart; (B) administering to the subject two or more subsequent doses of an effective amount of the fusion protein, wherein the dosing interval for the two or more subsequent doses is between about 21 to 36 days apart; (C) pausing administration of the fusion protein to the subject for a period of between about 6 weeks to about 8 months; and (D) repeating the administration of any one of steps A and B, wherein (a) at least 5 amino acids are truncated from the C-terminus of the FLT3L extracellular domain; and/or (b) the Fc region does not comprise a hinge region. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27. In some embodiments, the fusion protein comprises an amino acid sequence selected from the group consisting of SEQ ID NOs: 1-18 and 21-27. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 14. In some embodiments, the fusion protein comprises the amino acid sequence of SEQ ID NO: 14. In some embodiments, the method further comprises co-administering to the subject an anti-cancer agent. In some embodiments, the anti-cancer agent is sacituzumab govitecan. In some embodiments, the anti-cancer agent is magrolimab. In some embodiments, the anti-cancer agent is an anti-CD47 antibody. In some embodiments, the anti-cancer agent is an inhibitor of MCL-1. In some embodiments, the method further comprises co-administering to the subject an immunotherapy. In some embodiments, the method further comprises co-administering to the subject an immune checkpoint protein or receptor. In some embodiments, the method further comprises co-administering to the subject an inhibitor of an immune checkpoint protein or receptor. In some embodiments, the method further comprises co-administering to the subject an activator of an immune checkpoint protein or receptor.

Provided herein are methods of preventing, reducing and/or inhibiting the recurrence, growth, proliferation, migration and/or metastasis of a cancer cell or population of cancer cells in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

Provided herein are methods of treating and/or inhibiting cancer in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

Provided herein are methods of enhancing, promoting, and/or increasing the tumor infiltration of T-cells and/or NK cells in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

Provided herein are methods of enhancing, promoting, and/or accelerating the recovery from or reversing the effects of lymphopenia in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

Provided herein are methods of promoting, inducing and/or increasing the expansion and/or proliferation of a cell or a population of cells that express fms related tyrosine kinase 3 (FLT3, CD135) in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

Provided herein are methods of inducing an immune system in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

Provided herein are methods of preventing, reducing and/or inhibiting the recurrence, growth, proliferation, migration and/or metastasis of a cancer cell or population of cancer cells in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor.

Provided herein are methods of treating and/or inhibiting in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor.

Provided herein are methods of enhancing, promoting, and/or increasing the tumor infiltration of T-cells and/or NK cells in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor.

Provided herein are methods of enhancing, promoting, and/or accelerating the recovery from or reversing the effects of lymphopenia in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor.

Provided herein are methods of promoting, inducing and/or increasing the expansion and/or proliferation of a cell or a population of cells that express fms related tyrosine kinase 3 (FLT3, CD135) in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor.

Provided herein are methods of inducing the immune system in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor.

Provided herein are methods of preventing, reducing and/or inhibiting the recurrence, growth, proliferation, migration and/or metastasis of a cancer cell or population of cancer cells in a subject in need thereof, comprising administering to the subject (I) an effective amount of a human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor.

Provided herein are methods of treating and/or inhibiting in a subject in need thereof, comprising administering to the subject (I) an effective amount of a human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor.

Provided herein are methods of enhancing, promoting, and/or increasing the tumor infiltration of T-cells and/or NK cells in a subject in need thereof, comprising administering to the subject (I) an effective amount of a human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor.

Provided herein are methods of enhancing, promoting, and/or accelerating the recovery from or reversing the effects of lymphopenia in a subject in need thereof, comprising administering to the subject (I) an effective amount of a human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor.

Provided herein are methods of promoting, inducing and/or increasing the expansion and/or proliferation of a cell or a population of cells that express fms related tyrosine kinase 3 (FLT3, CD135) in a subject in need thereof, comprising administering to the subject (I) an effective amount of a human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor.

Provided herein are methods of inducing the immune system in a subject in need thereof, comprising administering to the subject (I) an effective amount of a human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor.

In some embodiments, for any of the methods disclosed herein, no more than 30000 μg, 29000 μg, 28000 μg, 27000 μg, 26000 μg, 25000 μg, 24000 μg, 23000 μg, 22000 μg, 21000 μg, 20000 μg, 19000 μg, 18000 μg, 17000 μg, 16000 μg, 15000 μg, 14000 μg, 13000 μg, 12000 μg, 11000 μg, 10000 μg, 9000 μg, 8000 μg, 7000 μg, 6000 μg, or 5000 μg of the fusion protein is administered to the subject per dose.

In some embodiments, for any of the methods disclosed herein, between about 600 μg to about 30000 μg, about 600 μg to about 29000 μg, about 600 μg to about 28000 μg, about 600 μg to about 27000 μg, about 600 μg to about 26000 μg, about 600 μg to about 25000 μg, about 600 μg to about 24000 μg, about 600 μg to about 23000 μg, about 600 μg to about 22000 μg, about 600 μg to about 21000 μg, about 600 μg to about 20000 μg, about 600 μg to about 19000 μg, about 600 μg to about 18000 μg, about 600 μg to about 17000 μg, about 600 μg to about 16000 μg, about 600 μg to about 15000 μg, about 600 μg to about 14000 μg, about 600 μg to about 13000 μg, about 600 μg to about 12000 μg, about 600 μg to about 11000 μg, about 600 μg to about 10000 μg, about 1000 μg to about 30000 μg, about 1000 μg to about 29000 μg, about 1000 μg to about 28000 μg, about 1000 μg to about 27000 μg, about 1000 μg to about 26000 μg, about 1000 μg to about 25000 μg, about 1000 μg to about 24000 μg, about 1000 μg to about 23000 μg, about 1000 μg to about 22000 μg, about 1000 μg to about 21000 μg, about 1000 μg to about 20000 μg, about 1000 μg to about 19000 μg, about 1000 μg to about 18000 μg, about 1000 μg to about 17000 μg, about 1000 μg to about 16000 μg, about 1000 μg to about 15000 μg, about 1000 μg to about 14000 μg, about 1000 μg to about 13000 μg, about 1000 μg to about 12000 μg, about 1000 μg to about 11000 μg, about 1000 μg to about 10000 μg, about 2000 μg to about 30000 μg, about 2000 μg to about 29000 μg, about 2000 μg to about 28000 μg, about 2000 μg to about 27000 μg, about 2000 μg to about 26000 μg, about 2000 μg to about 25000 μg, about 2000 μg to about 24000 μg, about 2000 μg to about 23000 μg, about 2000 μg to about 22000 μg, about 2000 μg to about 21000 μg, about 2000 μg to about 20000 μg, about 2000 μg to about 19000 μg, about 2000 μg to about 18000 μg, about 2000 μg to about 17000 μg, about 2000 μg to about 16000 μg, about 2000 μg to about 15000 μg, about 2000 μg to about 14000 μg, about 2000 μg to about 13000 μg, about 2000 μg to about 12000 μg, about 2000 μg to about 11000 μg, or about 2000 μg to about 10000 μg of the fusion protein is administered to the subject per dose.

In some embodiments, for any of the methods disclosed herein, at least about 225 μg, 250 μg, 275 μg, 300 μg, 400 μg, 500 μg, 600 μg, 625 μg, 650 μg, 675 μg, 700 μg, 800 μg, 900 μg, 1000 μg, 1100 μg, 1200 μg, 1300 μg, 1400 μg, 1500 μg, 1600 μg, 1700 μg, 1800 μg, 1900 μg, 2000 μg, 2100 μg, 2200 μg, 2300 μg, 2400 μg, 2500 μg, 2600 μg, 2700 μg, 2800 μg, 2900 μg, or 3000 μg of the fusion protein is administered to the subject per dose. In some embodiments, for any of the methods disclosed herein, at least about 800 μg of the fusion protein is administered to the subject per dose. In some embodiments, for any of the methods disclosed herein, at least about 1000 μg of the fusion protein is administered to the subject per dose. In some embodiments, for any of the methods disclosed herein, at least about 1500 μg of the fusion protein is administered to the subject per dose. In some embodiments, for any of the methods disclosed herein, at least about 2000 μg of the fusion protein is administered to the subject per dose. In some embodiments, for any of the methods disclosed herein, at least about 2500 μg of the fusion protein is administered to the subject per dose. In some embodiments, for any of the methods disclosed herein, at least about 3000 μg of the fusion protein is administered to the subject per dose.

In some embodiments, for any of the methods disclosed herein, at least two doses of the fusion protein are administered at least 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days apart. In some embodiments, for any of the methods disclosed herein, at least two doses of the fusion protein are administered at least 10 days apart. In some embodiments, for any of the methods disclosed herein, at least two doses of the fusion protein are administered at least 14 days apart. In some embodiments, for any of the methods disclosed herein, at least two doses of the fusion protein are administered at least 21 days apart. In some embodiments, for any of the methods disclosed herein, at least two doses of the fusion protein are administered at least 28 days apart. In some embodiments, for any of the methods disclosed herein, (i) at least two doses of the fusion protein are administered at least 10 days apart; and (ii) at least two additional doses of the fusion protein are administered at least 21 days apart. In some embodiments, for any of the methods disclosed herein, (i) at least two doses of the fusion protein are administered at least 14 days apart; and (ii) at least two additional doses of the fusion protein are administered at least 21 days apart. In some embodiments, for any of the methods disclosed herein, (i) at least two doses of the fusion protein are administered at least 14 days apart; and (ii) at least two additional doses of the fusion protein are administered at least 28 days apart.

In some embodiments, for any of the methods disclosed herein, at least two doses of the fusion protein are administered at least 1, 2, 3, 4, 5, 6, 7, 8, 9, 10, 11, 12, 13, 14, 15, 16, 17, 18, 19, or 20 weeks apart. In some embodiments, for any of the methods disclosed herein, at least two doses of the fusion protein are administered at least 1 week apart. In some embodiments, for any of the methods disclosed herein, at least two doses of the fusion protein are administered at least 2 weeks apart. In some embodiments, for any of the methods disclosed herein, at least two doses of the fusion protein are administered at least 3 weeks apart. In some embodiments, for any of the methods disclosed herein, at least two doses of the fusion protein are administered at least 4 weeks apart. In some embodiments, for any of the methods disclosed herein, (i) at least two doses of the fusion protein are administered at least 1 week apart; and (ii) at least two additional doses of the fusion protein are administered at least 3 weeks apart. In some embodiments, for any of the methods disclosed herein, (i) at least two doses of the fusion protein are administered at least 2 weeks apart; and (ii) at least two additional doses of the fusion protein are administered at least 3 weeks apart. In some embodiments, for any of the methods disclosed herein, (i) at least two doses of the fusion protein are administered at least 2 weeks apart; and (ii) at least two additional doses of the fusion protein are administered at least 4 weeks apart.

In some embodiments, for any of the methods disclosed herein, the method further comprises pausing administration of the fusion protein for at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 weeks. In some embodiments, for any of the methods disclosed herein, administration of the fusion protein is paused for at least about 8 weeks. In some embodiments, for any of the methods disclosed herein, administration of the fusion protein is paused for at least about 10 weeks. In some embodiments, for any of the methods disclosed herein, administration of the fusion protein is paused for at least about 12 weeks. In some embodiments, for any of the methods disclosed herein, administration of the fusion protein is paused for at least about 14 weeks. In some embodiments, for any of the methods disclosed herein, administration of the fusion protein is paused for at least about 16 weeks. In some embodiments, for any of the methods disclosed herein, administration of the fusion protein is paused for at least about 18 weeks. In some embodiments, for any of the methods disclosed herein, administration of the fusion protein is paused for at least about 20 weeks. In some embodiments, for any of the methods disclosed herein, the method further comprises pausing administration of the fusion protein for at least about 1, 2, 3, 4, 5, 6, 7, 8, 9, 10, 11, or 12 months. In some embodiments, for any of the methods disclosed herein, administration of the fusion protein is paused for at least about 2 months. In some embodiments, for any of the methods disclosed herein, administration of the fusion protein is paused for at least about 3 months. In some embodiments, for any of the methods disclosed herein, administration of the fusion protein is paused for at least about 4 months. In some embodiments, for any of the methods disclosed herein, administration of the fusion protein is paused for at least about 5 months. In some embodiments, for any of the methods disclosed herein, administration of the fusion protein is paused for at least about 6 months.

In some embodiments, for any of the methods disclosed herein, at least about 2, 3, 4, 5, 6, 7, 8, 9, 10, 11, 12, 13, 14, 15, 16, 17, 18, 19, or 20 doses of the fusion protein are administered to the subject prior to pausing administration of the fusion protein. In some embodiments, for any of the methods disclosed herein, at least about 3 doses of the fusion protein are administered to the subject prior to pausing administration of the fusion protein. In some embodiments, for any of the methods disclosed herein, at least about 4 doses of the fusion protein are administered to the subject prior to pausing administration of the fusion protein. In some embodiments, for any of the methods disclosed herein, at least about 5 doses of the fusion protein are administered to the subject prior to pausing administration of the fusion protein. In some embodiments, for any of the methods disclosed herein, at least about 6 doses of the fusion protein are administered to the subject prior to pausing administration of the fusion protein. In some embodiments, for any of the methods disclosed herein, at least about 7 doses of the fusion protein are administered to the subject prior to pausing administration of the fusion protein. In some embodiments, for any of the methods disclosed herein, at least about 8 doses of the fusion protein are administered to the subject prior to pausing administration of the fusion protein. In some embodiments, for any of the methods disclosed herein, less than about 20, 19, 18, 17, 16, 15, 14, 13, 12, 11, 10, or 9 doses of the fusion protein are administered to the subject prior to pausing administration of the fusion protein. In some embodiments, for any of the methods disclosed herein, less than about 12 doses of the fusion protein are administered to the subject prior to pausing administration of the fusion protein. In some embodiments, for any of the methods disclosed herein, less than about 10 doses of the fusion protein are administered to the subject prior to pausing administration of the fusion protein. In some embodiments, for any of the methods disclosed herein, less than about 8 doses of the fusion protein are administered to the subject prior to pausing administration of the fusion protein. In some embodiments, for any of the methods disclosed herein, about 2 to about 15, about 2 to about 12, about 2 to about 10, about 2 to about 8, about 3 to about 15, about 3 to about 12, about 3 to about 10, about 3 to about 8, about 4 to about 15, about 4 to about 12, about 4 to about 10, about 4 to about 8, about 5 to about 15, about 5 to about 12, about 5 to about 10, about 5 to about 8, about 6 to about 15, about 6 to about 12, about 6 to about 10, about 6 to about 8 doses of the fusion protein are administered to the subject prior to pausing administration of the fusion protein. In some embodiments, for any of the methods disclosed herein, about 2 to about 10 doses of the fusion protein are administered to the subject prior to pausing administration of the fusion protein. In some embodiments, for any of the methods disclosed herein, about 3 to about 12 doses of the fusion protein are administered to the subject prior to pausing administration of the fusion protein. In some embodiments, for any of the methods disclosed herein, about 3 to about 9 doses of the fusion protein are administered to the subject prior to pausing administration of the fusion protein. In some embodiments, for any of the methods disclosed herein, about 4 to about 12 doses of the fusion protein are administered to the subject prior to pausing administration of the fusion protein. In some embodiments, for any of the methods disclosed herein, about 4 to about 9 doses of the fusion protein are administered to the subject prior to pausing administration of the fusion protein.

In some embodiments, for any of the methods disclosed herein, a plurality of doses of the fusion protein is administered over a duration of at least about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks. In some embodiments, for any of the methods disclosed herein, the plurality of doses of the fusion protein is administered over a duration of at least about 6 weeks. In some embodiments, for any of the methods disclosed herein, the plurality of doses of the fusion protein is administered over a duration of at least about 8 weeks. In some embodiments, for any of the methods disclosed herein, the plurality of doses of the fusion protein is administered over a duration of at least about 10 weeks. In some embodiments, for any of the methods disclosed herein, the plurality of doses of the fusion protein is administered over a duration of at least about 14 weeks. In some embodiments, for any of the methods disclosed herein, the plurality of doses of the fusion protein is administered over a duration of at least about 18 weeks. In some embodiments, for any of the methods disclosed herein, the plurality of doses of the fusion protein is administered over a duration of at least about 20 weeks. In some embodiments, for any of the methods disclosed herein, the plurality of doses of the fusion protein is administered over a duration of at least about 30 weeks. In some embodiments, for any of the methods disclosed herein, a plurality of doses of the fusion protein is administered over a duration of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 months. In some embodiments, for any of the methods disclosed herein, the plurality of doses of the fusion protein is administered over a duration of at least about 2 months. In some embodiments, for any of the methods disclosed herein, the plurality of doses of the fusion protein is administered over a duration of at least about 3 months. In some embodiments, for any of the methods disclosed herein, the plurality of doses of the fusion protein is administered over a duration of at least about 4 months. In some embodiments, for any of the methods disclosed herein, the plurality of doses of the fusion protein is administered over a duration of at least about 6 months. In some embodiments, for any of the methods disclosed herein, the plurality of doses of the fusion protein is administered over a duration of at least about 8 months. In some embodiments, for any of the methods disclosed herein, the plurality of doses of the fusion protein is administered over a duration of at least about 10 months. In some embodiments, for any of the methods disclosed herein, the plurality of doses of the fusion protein is administered over a duration of at least about 12 months. In some embodiments, for any of the methods disclosed herein, the plurality of doses of the fusion protein is administered over a duration of at least about 14 months.

In some embodiments, for any of the methods disclosed herein, the fusion protein is administered to the subject via intravenous administration.

In some embodiments, for any of the methods disclosed herein, the fusion protein is administered to the subject via subcutaneous administration.

In some embodiments, the methods disclosed herein promote, induce and/or increase the expansion and/or proliferation of a cell or a population of cells that express fms related tyrosine kinase 3 (FLT3, CD135). In some embodiments, the cell or population of cells that express FLT3 comprise dendritic cells (e.g., cDC1 cells and/or cDC2 cells), monocyte-derived dendritic cells (moDCs), and/or progenitor cells thereof. In some embodiments, the cell or population of cells that express FLT3 comprise hematopoietic progenitor cells. In some embodiments, the hematopoietic progenitor cells are selected from the group consisting of: Common Lymphoid Progenitors (CLPs), Early Progenitors with Lymphoid and Myeloid potential (EPLMs), granulocyte-monocyte (GM) progenitors (GMP), monocyte-derived dendritic cells (moDCs) progenitors, and early multi-potent progenitors (MPP) within the Lineage⁻kit⁺Sca1− (LSK) compartment. In some embodiments, the cell or population of cells are expanded within a solid tumor. In some embodiments, conventional dendritic cells (e.g., cDC1 and/or cDC2) are expanded or induced to proliferate. In some embodiments, cDC1 dendritic cells (e.g., positive for surface expression of X-C motif chemokine receptor 1 (XCR1), thrombomodulin (THBD, CD141), and C-type lectin domain containing 9A (CLEC9A)) are expanded or induced to proliferate. In some embodiments, cDC2 dendritic cells (e.g., positive for surface expression of CD1c molecule (BDCA) are expanded or induced to proliferate.

In some embodiments, for any of the methods disclosed herein, administering the fusion protein comprises administering a polynucleotide encoding the fusion protein. In some embodiments, the polynucleotide is selected from the group consisting of DNA, cDNA, RNA or mRNA. In some embodiments, the polynucleotide comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from the group consisting of SEQ ID NOs: 28-70. In some embodiments, the polynucleotide comprises a nucleic acid selected from the group consisting of SEQ ID NOs: 28-70. In some embodiments, the polynucleotide is delivered via a vector. In some embodiments, the vector is a plasmid vector or a viral vector. In some embodiments, the viral vector comprises an oncolytic viral vector. In some embodiments, the viral vector comprises a DNA virus or a RNA virus. In some embodiments, the viral vector is from a viral family selected from the group consisting of: Adenoviridae (e.g., Adenovirus), Arenaviridae (e.g., lymphocytic choriomeningitis mammarenavirus, Cali mammarenavirus (a.k.a., Pichinde mammarenavirus), Poxviridae (e.g., Vaccinia virus), Herpesviridae (e.g., Herpesvirus, e.g., HSV-1), Parvoviridae (e.g., Parvovirus H1), Reoviridae (e.g., Reovirus), Picornaviridae (e.g., Coxsackievirus, Seneca Valley Virus, Poliovirus), Paramyxoviridae (e.g., Measles virus, Newcastle disease virus (NDV)), Rhabdoviridae (e.g., Vesicular stomatitis virus (VSV)), Togaviridae (e.g., Alphavirus, Sindbis virus), Enteroviridae (e.g., Echovirus).

In some embodiments, for any of the methods disclosed herein, the fusion protein is delivered as a homodimer comprising two identical fusion proteins.

In some embodiments, for any of the methods disclosed herein, the fusion protein is delivered as a heterodimer comprising two non-identical fusion proteins.

In some embodiments, for any of the methods disclosed herein, the fusion protein is delivered as a heterodimer comprising the fusion protein and a second fusion protein comprising a targeting moiety domain fused to a second Fc region.

In some embodiments, for any of the methods disclosed herein, the fusion protein is formulated for delivery via a lipid nanoparticle, micelle, liposome, or capsule. In some embodiments, for any of the methods disclosed herein, the fusion protein is formulated for delivery via a lipid nanoparticle.

In some embodiments, any of the methods disclosed herein further comprise co-administering to the subject an anticancer agent. In some embodiments, the anticancer agent is an anti-neoplastic or chemotherapeutic agent.

In some embodiments, any of the methods disclosed herein further comprise co-administering to the subject an immunotherapy. In some embodiments, the immunotherapy comprises co-administering one or more antibodies or antigen-binding antibody fragments thereof, or antibody-drug conjugates thereof, CD3-targeting multi-specific molecules, NK cell-activating receptor-targeting multi-specific molecules, or non-immunoglobulin antigen-binding domains or antibody mimetic proteins directed against one or more targets or tumor associated antigens (TAAs).

In some embodiments, for any of the methods disclosed herein, the subject receives radiation therapy. In some embodiments, the radiation therapy comprises stereotactic body radiation therapy (SBRT).

In some embodiments, any of the methods disclosed herein further comprise co-administering the fusion protein with an anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, radiation therapy, or any combination thereof. In some embodiments, the fusion protein is administered prior to co-administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRP-alpha (SIRPα) targeting agent, and/or radiation therapy. In some embodiments, the fusion protein is administered at least 1, 2, 3, 4, 5, 6, 7, 8, 9, or 10 days prior to administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy. In some embodiments, the fusion protein is administered after administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy. In some embodiments, the fusion protein is administered at least 1, 2, 3, 4, 5, 6, 7, 8, 9, or 10 days after administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy. In some embodiments, the fusion protein is administered concurrently with administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy. In some embodiments, the fusion protein is administered within 10, 15, 20, 25, 30, 35, 40, 45, 50, 55, 60, 65, 70, 75, 80, 85, 90, 95, 100, 110, 120, 130, 140, 150, 160, 170, 180, 190, or 200 minutes of administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy. In some embodiments, the fusion protein is administered within 1, 2, 3, 4, 5, 6, 7, 8, 9, 10, 11, 12, 13, 14, or 15 hours of administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy.

In some embodiments, for any of the methods disclosed herein, the subject has cancer. In some embodiments, for any of the methods disclosed herein, the subject is in cancer remission. In some embodiments, for any of the methods disclosed herein, the subject has a hematological cancer, e.g., a leukemia (e.g., Acute Myelogenous Leukemia (AML), Acute Lymphoblastic Leukemia (ALL), B-cell ALL, Myelodysplastic Syndrome (MDS), myeloproliferative disease (MPD), Chronic Myelogenous Leukemia (CIVIL), Chronic Lymphocytic Leukemia (CLL), undifferentiated leukemia), a lymphoma (e.g., small lymphocytic lymphoma (SLL), mantle cell lymphoma (MCL), follicular lymphoma (FL), T-cell lymphoma, B-cell lymphoma, diffuse large B-cell lymphoma (DLBCL), marginal zone lymphoma (MZL), Waldestrom's macroglobulinemia (WM)) and/or a myeloma (e.g., multiple myeloma (MM)). In some embodiments, for any of the methods disclosed herein, the subject has a solid tumor. In some embodiments, the solid tumor is a malignant tumor. In some embodiments, the solid tumor is a metastatic tumor. In some embodiments, for any of the methods disclosed herein, the subject has a tumor infiltrated with conventional dendritic cells (cDC1).

In some embodiments, for any of the methods disclosed herein, the subject has a cancer selected from the group consisting of a lung cancer, a colorectal cancer, a breast cancer, a prostate cancer, a cervical cancer and a head and neck cancer.

In some embodiments, for any of the methods disclosed herein, the subject has neutropenia or lymphopenia.

In some embodiments, for any of the methods disclosed herein, the subject has received a lymphodepleting chemotherapy regimen.

In some embodiments, for any of the methods disclosed herein, the subject is naïve to or has not received chemotherapy.

In some embodiments, for any of the methods disclosed herein, the subject has bone marrow cells, or is not depleted of bone marrow cells.

In some embodiments, for any of the methods disclosed herein, the subject does not have a mutation in the gene encoding the FLT3 receptor that causes or results in or is associated with cancer.

In some embodiments, the methods disclosed herein induce the immune system in a subject in need thereof. In some embodiments, the subject is suffering from a virus infection. In some embodiments, the virus infection is caused by a virus selected from the group consisting of hepatitis B virus, human immunodeficiency virus (HIV), and coronavirus. In some embodiments, the coronavirus is selected from the group consisting of Severe Acute Respiratory Syndrom (SARS)-associated virus, Middle East Respiratory Syndrom (MFRS)-associated virus, and COVID-19 virus (SARS-CoV-2).

### BRIEF DESCRIPTION OF THE DRAWINGS

FIG. 1 illustrates induction of mouse IL-6 in a mouse FLT3-expressing M1 cell line by a titration (50-0.00005 nM) of recombinant human FLT3-ligand (Recombinant huFLT3L, closed circle), recombinant human FLT3-ligand human IgG1 fusion protein (Recombinant huFLT3L-Fc, open triangle), anti-mouse FLT3 agonist antibody (Comparator 1, closed triangle) or human IgG1 isotype antibody (Isotype negative control, open square). The x-axis shows the protein concentration (nM) and the y-axis shows mouse IL-6 concentration (pg/mL). The cross symbol indicates the IL-6 baseline level in untreated cells. Graph is a combination of two independent experiments. Experiments were performed in duplicate. Error bars represent standard deviation of the mean values. EC50 values are shown in Table 1.

FIG. 2 illustrates proliferation of a human FLT3-expressing AML5 cell line in response to a titration (100-0.0025 nM) of recombinant human FLT3-ligand (Recombinant huFLT3L, open square), human FLT3-ligand human hingeless IgG1 fusion protein (SEQ ID NO:1, open triangle), human FLT3-ligand human IgG1 fusion protein (SEQ ID NO:21, open circle) or human IgG1 isotype antibody (hIgG1 Isotype, cross). The x-axis shows the protein concentration (nM) and the y-axis shows the relative luminescence units (RLU). Graph is a result of one experiment. Experiments were performed in triplicate. Error bars represent standard deviation of the mean values. EC50 values are shown in Table 2.

FIG. 3 illustrates proliferation of a human FLT3-expressing AML5 cell line induced by a titration (5-0.00008 nM) of human wildtype FLT3-ligand human hingeless IgG1 fusion protein (SEQ ID NO:1, open triangle), human FLT3-ligand (H8Y) human hingeless IgG1 fusion protein (SEQ ID NO:22, open circle), human FLT3-ligand (K84E) human hingeless IgG1 fusion protein (SEQ ID NO:23, open square), human FLT3-ligand (H8Y+K84E) human hingeless IgG1 fusion protein (SEQ ID NO:24, closed circle), or human IgG1 isotype antibody (hIgG1 Isotype, cross). The x-axis shows the protein concentration (nM) and the y-axis shows the relative luminescence units (RLU). Graph is a combination of two independent experiments. Experiments were performed in triplicate. Error bars represent standard deviation of the mean values. EC50 values are shown in Table 3.

FIG. 4 illustrates proliferation of a human FLT3-expressing AML5 cell line induced by a titration (10-0.0004 nM) of human FLT3-ligand human hingeless IgG1 fusion protein (SEQ ID NO:1, open triangle), mouse FLT3-ligand mouse IgG2a (LALA-PG) fusion protein (SEQ ID NO:19, open circle), mouse FLT3-ligand mouse IgG2a (C136S LALA-PG) fusion protein (SEQ ID NO:20, open square), or human IgG1 isotype antibody (hIgG1 Isotype, cross). The x-axis shows the protein concentration (nM) and the y-axis shows the relative luminescence units (RLU). Graph is a result of one experiment of. Experiment was performed in triplicate. Error bars represent standard deviation of the mean values. EC50 values are shown in Table 4.

FIG. 5 illustrates proliferation of a human FLT3-expressing AML5 cell line induced by a titration (10-0.0004 nM) of human FLT3-ligand human hingeless IgG1 fusion protein (SEQ ID NO:1, open triangle), human FLT3-ligand (Δ5 amino acid) human hingeless IgG1 fusion protein (SEQ ID NO:2, closed triangle), human FLT3-ligand human IgG4 (S228P/L235E) fusion protein (SEQ ID NO:3, open circle), human FLT3-ligand human IgG4 (S228P/F234A/L235A) fusion protein (SEQ ID NO:4, closed circle), human FLT3-ligand (S128A/S151A) human hingeless IgG1 fusion protein (SEQ ID NO:5, open square), human FLT3-ligand (Δ5 amino acid) human IgG4 (S228P/F234A/L235A) fusion protein (SEQ ID NO:6, closed square), human FLT3-ligand (Δ10 amino acid) human hingeless IgG1 fusion protein (SEQ ID NO:7, cross), or human FLT3-ligand (Δ10 amino acid) human IgG4 (S228P/F234A/L235A) fusion protein (SEQ ID NO:8, closed diamond). The x-axis shows the protein concentration (nM) and the y-axis shows the relative luminescence units (RLU). Graph is a combination of two independent experiments. Experiment was performed in triplicate. Error bars represent standard deviation of the mean values. EC50 values are shown in Table 5.

FIG. 6 illustrates binding to recombinant human FLT3 of a titration (35-0.0001 nM) of human FLT3-ligand human hingeless IgG1 fusion protein (SEQ ID NO:1, open triangle), human FLT3-ligand (Δ5 amino acid) human hingeless IgG1 fusion protein (SEQ ID NO:2, closed triangle), human FLT3-ligand human IgG4 (S228P/L235E) fusion protein (SEQ ID NO:3, open circle), human FLT3-ligand human IgG4 (S228P/F234A/L235A) fusion protein (SEQ ID NO:4, closed circle), human FLT3-ligand (S128A/S151A) human hingeless IgG1 fusion protein (SEQ ID NO:5, open square), human FLT3-ligand (Δ5 amino acid) human IgG4 (S228P/F234A/L235A) fusion protein (SEQ ID NO:6, closed square), human FLT3-ligand (Δ10 amino acid) human hingeless IgG1 fusion protein (SEQ ID NO:7, cross), or human FLT3-ligand (Δ10 amino acid) human IgG4 (S228P/F234A/L235A) fusion protein (SEQ ID NO:8, closed diamond). The x-axis shows the protein concentration (nM) and the y-axis shows the optical density (OD) at 450 nm. Graph is a result of one experiment. EC50 values are shown in Table 6.

FIG. 7 illustrates binding of recombinant human FcRn with a dose titration (235-0.035 nM) of human FLT3-ligand human hingeless IgG1 fusion protein (SEQ ID NO:1, open triangle), human FLT3-ligand (Δ5 amino acid) human hingeless IgG1 fusion protein (SEQ ID NO:2, closed triangle), human FLT3-ligand human IgG4 (S228P/L235E) fusion protein (SEQ ID NO:3, open circle), human FLT3-ligand human IgG4 (S228P/F234A/L235A) fusion protein (SEQ ID NO:4, closed circle), human FLT3-ligand (S128A/S151A) human hingeless IgG1 fusion protein (SEQ ID NO:5, open square), human FLT3-ligand (Δ5 amino acid) human IgG4 (S228P/F234A/L235A) fusion protein (SEQ ID NO:6, closed square), human FLT3-ligand (Δ10 amino acid) human hingeless IgG1 fusion protein (SEQ ID NO:7, cross), human FLT3-ligand (Δ10 amino acid) human IgG4 (S228P/F234A/L235A) fusion protein (SEQ ID NO:8, closed diamond), human IgG1 isotype antibody (open diamond), or human IgG4 isotype antibody (open star). The x-axis shows the protein concentration (nM) and the y-axis shows the optical density (OD) at 450-650 nm. Graph is a result of one experiment. Experiment was performed in duplicate. Error bars represent standard deviation of the mean values. Estimated EC50 values are shown in Table 7.

FIG. 8 illustrates binding of human IgG to recombinant human FcγRI competed with a titration (294-0.48 nM) of human FLT3-ligand human hingeless IgG1 fusion protein (SEQ ID NO:1, open triangle), human FLT3-ligand (Δ5 amino acid) human hingeless IgG1 fusion protein (SEQ ID NO:2, closed triangle), human FLT3-ligand human IgG4 (S228P/L235E) fusion protein (SEQ ID NO:3, open circle), human FLT3-ligand human IgG4 (S228P/F234A/L235A) fusion protein (SEQ ID NO:4, closed circle), human FLT3-ligand (S128A/S151A) human hingeless IgG1 fusion protein (SEQ ID NO:5, open square), human FLT3-ligand (Δ5 amino acid) human IgG4 (S228P/F234A/L235A) fusion protein (SEQ ID NO:6, closed square), human FLT3-ligand (Δ10 amino acid) human hingeless IgG1 fusion protein (SEQ ID NO:7, cross), human FLT3-ligand (Δ10 amino acid) human IgG4 (S228P/F234A/L235A) fusion protein (SEQ ID NO:8, closed diamond), human IgG1 isotype antibody (open diamond), or human IgG4 isotype antibody (open star). The x-axis shows the protein concentration (nM) and the y-axis shows the relative fluorescence units (RFU). Graph is a result of one experiment. Experiment was performed in duplicate. Error bars represent standard deviation of the mean values. EC50 values are shown in Table 8.

FIG. 9 illustrates binding of human IgG to recombinant human FcγRIIIa (V-variant) competed with a dose titration (1176-1.92 nM) of human FLT3-ligand human hingeless IgG1 fusion protein (SEQ ID NO:1, open triangle), human FLT3-ligand (Δ5 amino acid) human hingeless IgG1 fusion protein (SEQ ID NO:2, closed triangle), human FLT3-ligand human IgG4 (S228P/L235E) fusion protein (SEQ ID NO:3, open circle), human FLT3-ligand human IgG4 (S228P/F234A/L235A) fusion protein (SEQ ID NO:4, closed circle), human FLT3-ligand (S128A/S151A) human hingeless IgG1 fusion protein (SEQ ID NO:5, open square), human FLT3-ligand (Δ5 amino acid) human IgG4 (S228P/F234A/L235A) fusion protein (SEQ ID NO:6, closed square),), human FLT3-ligand (Δ10 amino acid) human hingeless IgG1 fusion protein (SEQ ID NO:7, cross), human FLT3-ligand (Δ10 amino acid) human IgG4 (S228P/F234A/L235A) fusion protein (SEQ ID NO:8, closed diamond), human IgG1 isotype antibody (open diamond), or human IgG4 isotype antibody (open star). The x-axis shows the protein concentration (nM) and the y-axis shows the relative fluorescence units (RFU). Graph is a result of one experiment. Experiment was performed in duplicate. Error bars represent standard deviation of the mean values. EC50 values are shown in Table 9.

FIG. 10 illustrates binding of recombinant human C1q to a titration (94-0.74 nM) of human FLT3-ligand human hingeless IgG1 fusion protein (SEQ ID NO:1, open triangle), human FLT3-ligand (Δ5 amino acid) human hingeless IgG1 fusion protein (SEQ ID NO:2, closed triangle), human FLT3-ligand human IgG4 (S228P/L235E) fusion protein (SEQ ID NO:3, open circle), human FLT3-ligand human IgG4 (S228P/F234A/L235A) fusion protein (SEQ ID NO:4, closed circle), human FLT3-ligand (S128A/S151A) human hingeless IgG1 fusion protein (SEQ ID NO:5, open square), human FLT3-ligand (Δ5 amino acid) human IgG4 (S228P/F234A/L235A) fusion protein (SEQ ID NO:6, closed square),), human FLT3-ligand (Δ10 amino acid) human hingeless IgG1 fusion protein (SEQ ID NO:7, cross), human FLT3-ligand (Δ10 amino acid) human IgG4 (S228P/F234A/L235A) fusion protein (SEQ ID NO:8, closed diamond), human IgG1 isotype antibody (open diamond), or human IgG4 isotype antibody (open star). The x-axis shows the protein concentration (nM) and the y-axis shows the optical density (OD) at 450-650 nm. Graph is a result of one experiment. Experiment was performed in duplicate. Error bars represent standard deviation of the mean values. EC50 values are shown in Table 10.

FIGS. 11A-11B illustrate a mouse serum concentration-time profile following 5 mg/kg intraperitoneal dosing of 8 FLT3-ligand fusion proteins relative to recombinant FLT3-ligand. Panel A: linear scale; Panel B: Log 10 scale after a single dose intravenous administration (5 mg/kg) of human FLT3-ligand human hingeless IgG1 fusion protein produced in Expi293 expression system (SEQ ID NO:1 Expi293, open triangle), human FLT3-ligand human hingeless IgG1 fusion protein produced in ExpiCHO expression system (SEQ ID NO:1 ExpiCHO, closed triangle), human FLT3-ligand (Δ5 amino acid) human hingeless IgG1 fusion protein (SEQ ID NO:2, open circle), human FLT3-ligand human IgG4 (S228P/L235E) fusion protein (SEQ ID NO:3, closed circle), human FLT3-ligand human IgG4 (S228P/F234A/L235A) fusion protein (SEQ ID NO:4, open square), human FLT3-ligand (S128A/S151A) human hingeless IgG1 fusion protein (SEQ ID NO:5, closed square), human FLT3-ligand (Δ5 amino acid) human IgG4 (S228P/F234A/L235A) fusion protein (SEQ ID NO:6, cross), human FLT3-ligand (Δ10 amino acid) human hingeless IgG1 fusion protein (SEQ ID NO:7, open diamond), human FLT3-ligand (Δ10 amino acid) human IgG4 (S228P/F234A/L235A) fusion protein (SEQ ID NO:8, closed diamond), or recombinant human FLT3-ligand (Recombinant huFLT3L, closed star). Graph is a result of one experiment. The x-axis shows days post injection and the y-axis shows protein concentration in serum (μg/mL). Each data point represents the mean value of 4 animals. Error bars represent standard deviation (SD) of the mean values. Mean pharmacokinetic values±SD are shown in Table 11.

FIG. 12 illustrates day 11 frequency of conventional dendritic cell subtype 1 (cDC1) in spleens of C57BL/6 mice administrated intravenously with 5 mg/kg of human FLT3-ligand human hingeless IgG1 fusion protein produced in Expi293 expression system (SEQ ID NO:1 Expi293, open triangle), human FLT3-ligand human hingeless IgG1 fusion protein produced in ExpiCHO expression system (SEQ ID NO:1 ExpiCHO, closed triangle), human FLT3-ligand (Δ5 amino acid) human hingeless IgG1 fusion protein (SEQ ID NO:2, open circle), human FLT3-ligand human IgG4 (S228P/L235E) fusion protein (SEQ ID NO:3, closed circle), human FLT3-ligand human IgG4 (S228P/F234A/L235A) fusion protein (SEQ ID NO:4, open square), human FLT3-ligand (S128A/S151A) human hingeless IgG1 fusion protein (SEQ ID NO:5, closed square), human FLT3-ligand (Δ5 amino acid) human IgG4 (S228P/F234A/L235A) fusion protein (SEQ ID NO:6, cross), human FLT3-ligand (Δ10 amino acid) human hingeless IgG1 fusion protein (SEQ ID NO:7, open diamond), human FLT3-ligand (Δ10 amino acid) human IgG4 (S228P/F234A/L235A) fusion protein (SEQ ID NO:8, closed diamond), or recombinant human FLT3-ligand (Recombinant huFLT3L, open star) at day 0. Baseline cDC1 frequency is indicated (closed star). The x-axis shows the percentage of splenic cDC1 in total mononuclear cells (MNCs). Graph is a result of one experiment. Each individual symbol represents the data point of a single mouse. Horizontal bars represent the mean values and the error bars represent standard deviation of the mean values. Mean frequency of each group is shown in Table 12.

FIG. 13 illustrates proliferation of a human FLT3-expressing AML5 cell line stimulated by a dose titration (10-0.0004 nM) of human FLT3-ligand human hingeless IgG1 fusion protein (SEQ ID NO:1, open triangle), human FLT3-ligand (Δ5 amino acid) human IgG4 (S228P/F234A/L235A) fusion protein (SEQ ID NO:6, open circle), human FLT3-ligand human hingeless IgG1 (M252Y/S254T/T256E) fusion protein (SEQ ID NO:9, open square), or human FLT3-ligand (Δ5 amino acid) human IgG4 (S228P/F234A/L235A/M252Y/S254T/T256E) fusion protein (SEQ ID NO:14, cross). The x-axis shows the protein concentration (nM) and the y-axis shows the relative luminescence units (RLU). Graph is a result of one experiment. Experiment was performed in duplicate. Error bars represent standard deviation of the mean values. EC50 values are shown in Table 13.

FIG. 14 illustrates differentiation of conventional dendritic cell subtype 1 (cDC1) cells from human bone marrow CD34+ stem cells by a dose titration (10-0.002 nM) of human FLT3-ligand human hingeless IgG1 fusion protein (SEQ ID NO:1, open triangle), human FLT3-ligand (Δ5 amino acid) human IgG4 (S228P/F234A/L235A) fusion protein (SEQ ID NO:6, open circle), human FLT3-ligand human hingeless IgG1 (M252Y/S254T/T256E) fusion protein (SEQ ID NO:9, open square), or human FLT3-ligand (Δ5 amino acid) human IgG4 (S228P/F234A/L235A/M252Y/S254T/T256E) fusion protein (SEQ ID NO:14, cross). The x-axis shows the protein concentration (nM) and the y-axis shows percentage of cDC1 in total mononuclear cells (MNCs). Graph is a summary of 13 bone marrow donors. Error bars represent standard error mean of the mean values. EC50 values are shown in Table 14.

FIG. 15 illustrates a dose titration (10-0.002 nM) potency of enhancing survival of PBMC-derived conventional dendritic cell subtype 1 (cDC1) cells by human FLT3-ligand human hingeless IgG1 fusion protein (SEQ ID NO:1, open triangle), human FLT3-ligand (45 amino acid) human IgG4 (S228P/F234A/L235A) fusion protein (SEQ ID NO:6, open circle), human FLT3-ligand human hingeless IgG1 (M252Y/S254T/T256E) fusion protein (SEQ ID NO:9, open square), or human FLT3-ligand (Δ5 amino acid) human IgG4 (S228P/F234A/L235A/M252Y/S254T/T256E) fusion protein (SEQ ID NO:14, cross). The x-axis shows the protein concentration (nM) and the y-axis shows percentage of cDC1 in total mononuclear cells (MNCs). Graph is a summary of 16 PBMC donors. Error bars represent standard error mean of the mean values. EC50 values are shown in Table 15.

FIG. 16 illustrates binding of recombinant human FLT3 to a titration (15-0.007 nM) of human FLT3-ligand human hingeless IgG1 fusion protein (SEQ ID NO:1, open triangle), human FLT3-ligand (Δ5 amino acid) human IgG4 (S228P/F234A/L235A) fusion protein (SEQ ID NO:6, open circle), human FLT3-ligand human hingeless IgG1 (M252Y/S254T/T256E) fusion protein (SEQ ID NO:9, open square), or human FLT3-ligand (Δ5 amino acid) human IgG4 (S228P/F234A/L235A/M252Y/S254T/T256E) fusion protein (SEQ ID NO:14, cross). The x-axis shows the protein concentration (nM) and the y-axis shows optical density (OD) at 450 nm. Graph is a result of one experiment. Experiment was performed in duplicate. Error bars represent standard deviation of the mean values. EC50 values are shown in Table 16.

FIG. 17 illustrates binding of recombinant human FcRn to a dose titration (3529-0.55 nM) of human FLT3-ligand human hingeless IgG1 fusion protein (SEQ ID NO:1, open triangle), human FLT3-ligand (Δ5 amino acid) human IgG4 (S228P/F234A/L235A) fusion protein (SEQ ID NO:6, open circle), human FLT3-ligand human hingeless IgG1 (M252Y/S254T/T256E) fusion protein (SEQ ID NO:9, open square), or human FLT3-ligand (Δ5 amino acid) human IgG4 (S228P/F234A/L235A/M252Y/S254T/T256E) fusion protein (SEQ ID NO:14, cross). The x-axis shows the protein concentration (nM) and the y-axis shows optical density (OD) at 450-650 nm. Graph is a result of one experiment. Experiment was performed in duplicate. Error bars represent standard deviation of the mean values. EC50 values are shown in Table 17.

FIG. 18 illustrates binding of human IgG to recombinant human FcγRI competed with a titration (294-0.48 nM) of human FLT3-ligand human hingeless IgG1 fusion protein (SEQ ID NO:1, open triangle), human FLT3-ligand (Δ5 amino acid) human IgG4 (S228P/F234A/L235A) fusion protein (SEQ ID NO:6, open circle), human FLT3-ligand human hingeless IgG1 (M252Y/S254T/T256E) fusion protein (SEQ ID NO:9, open square), human FLT3-ligand (Δ5 amino acid) human IgG4 (S228P/F234A/L235A/M252Y/S254T/T256E) fusion protein (SEQ ID NO:14, cross), human IgG1 isotype antibody (open diamond), or human IgG4 isotype antibody (open star). The x-axis shows the protein concentration (nM) and the y-axis shows the relative fluorescence units (RFU). Graph is a result of one experiment. Experiment was performed in duplicate. Error bars represent standard deviation of the mean values. EC50 values are shown in Table 18.

FIG. 19 illustrates binding of human IgG to recombinant human FcγRIIIa (V-variant) competed by a dose titration (1176-1.92 nM) of human FLT3-ligand human hingeless IgG1 fusion protein (SEQ ID NO:1, open triangle), human FLT3-ligand (Δ5 amino acid) human IgG4 (S228P/F234A/L235A) fusion protein (SEQ ID NO:6, open circle), human FLT3-ligand human hingeless IgG1 (M252Y/S254T/T256E) fusion protein (SEQ ID NO:9, open square), human FLT3-ligand (Δ5 amino acid) human IgG4 (S228P/F234A/L235A/M252Y/S254T/T256E) fusion protein (SEQ ID NO:14, cross), human IgG1 isotype antibody (open diamond), or human IgG4 isotype antibody (open star). The x-axis shows the protein concentration (nM) and the y-axis shows the relative fluorescence units (RFU). Graph is a result of one experiment. Experiment was performed in duplicate. Error bars represent standard deviation of the mean values. EC50 values are shown in Table 19.

FIG. 20 illustrates binding of recombinant human C1q to a titration (94-0.74 nM) of human FLT3-ligand human hingeless IgG1 fusion protein (SEQ ID NO:1, open triangle), human FLT3-ligand (Δ5 amino acid) human IgG4 (S228P/F234A/L235A) fusion protein (SEQ ID NO:6, open circle), human FLT3-ligand human hingeless IgG1 (M252Y/S254T/T256E) fusion protein (SEQ ID NO:9, open square), human FLT3-ligand (Δ5 amino acid) human IgG4 (S228P/F234A/L235A/M252Y/S254T/T256E) fusion protein (SEQ ID NO:14, cross), human IgG1 isotype antibody (open diamond), or human IgG4 isotype antibody (open star). The x-axis shows the protein concentration (nM) and the y-axis shows optical density (OD) at 450-650 nm. Graph is a result of one experiment. Experiment was performed in duplicate. Error bars represent standard deviation of the mean values. EC50 values are shown in Table 20.

FIGS. 21A-21B illustrate Cynomolgus macaque serum concentration-time profile following 500 μg/kg intravenous and subcutaneous dosing of 4 FLT3-ligand fusion proteins relative to recombinant FLT3-ligand. Average serum concentration-time profiles after intravenous (Panel A) or subcutaneous (Panel B) administration of human FLT3-ligand human hingeless IgG1 fusion protein (SEQ ID NO:1, open triangle), human FLT3-ligand (45 amino acid) human IgG4 (S228P/F234A/L235A) fusion protein (SEQ ID NO:6, open circle), human FLT3-ligand human hingeless IgG1 (M252Y/S254T/T256E) fusion protein (SEQ ID NO:9, open square), human FLT3-ligand (Δ5 amino acid) human IgG4 (S228P/F234A/L235A/M252Y/S254T/T256E) fusion protein (SEQ ID NO:14, cross). The x-axis shows days post injection and the y-axis shows protein concentration in serum (μg/mL). Each data point represents the mean value of 3 animals. Error bars represent standard deviation of the mean values. Mean pharmacokinetic values are shown in Table 21.

FIGS. 22A-22B illustrate kinetics of conventional dendritic cell subtype 1 (cDC1) fold-change in peripheral blood of Cynomolgus macaque administrated intravenously (Panel A) or subcutaneously (Panel B) with 500 μg/kg of human FLT3-ligand human hingeless IgG1 fusion protein (SEQ ID NO:1, open triangle), human FLT3-ligand (Δ5 amino acid) human IgG4 (S228P/F234A/L235A) fusion protein (SEQ ID NO:6, open circle), human FLT3-ligand human hingeless IgG1 (M252Y/S254T/T256E) fusion protein (SEQ ID NO:9, open square), human FLT3-ligand (Δ5 amino acid) human IgG4 (S228P/F234A/L235A/M252Y/S254T/T256E) fusion protein (SEQ ID NO:14, cross) at day 0. Graph is a result of one experiment. Each data point represents the mean value of 3 animals. Error bars represent standard deviation of the mean values.

FIG. 23 illustrates tumor growth kinetics of MC38 tumor-bearing C57BL/6 mice administrated intravenously with 3750 μg/kg (open triangle), 750 μg/kg (open circle), 150 μg/kg (open square), 30 μg/kg (cross) mouse FLT3-Ligand mouse IgG2a (C136S, LALA-PG), or 3753 μg/kg mouse IgG2a isotype (open star) at day 0 when tumor volume reached 50 mm³. The x-axis is days post dosing. The y-axis is tumor volume in mm³. Graph is representative of two independent experiments. Each data point represents the mean value of 7 animals. Error bars represent standard deviation of the mean values. Statistical differences in tumor growth rate of each dose group compared to the isotype group were determined by repeated mixed ANOVA mode. Data was fitted with a linear mixed effect model.

FIGS. 24A-24B illustrate day 7 quantification of conventional dendritic cell subtype 1 (cDC1) numbers in tumors (Panel A) or spleens (Panel B) of MC38 tumor-bearing C57BL/6 mice administrated intravenously with 3750 μg/kg (open triangle), 750 μg/kg (open circle), 150 μg/kg (open square), 30 μg/kg (cross) mouse FLT3-Ligand mouse IgG2a (C136S, LALA-PG), or 3753 μg/kg mouse IgG2a isotype (open star) at day 0 when tumor volume reached 50 mm³. The x-axis indicates dose groups. The y-axis shows the cDC1 number per gram of tumor (Panel A) or cDC1 number per spleen (Panel B). Graph is a result of one experiment. Each individual symbol represents the data point of a single mouse. Horizontal bars represent the mean values and the error bars represent standard deviation of the mean values. Statistical differences were determined one-way ANOVA with Dunnett's post-test. ****p-value<0.0001; ***p-value<0.001; **p-value<0.01.

FIG. 25 illustrates tumor growth kinetics of MC38 tumor-bearing C57BL/6 mice administrated intravenously with QW×2 dose of 3.75 μg/kg (open circle) mouse FLT3-Ligand mouse IgG2a (C136S, LALA-PG), Q3W dose of 3 mg/kg (open triangle) anti-mouse PD-1 (clone RMP1-14), combination of the two reagents (closed circle) or QW×2 dose of 10 mg/kg mouse IgG2a isotype control (open star) at day 0 when tumor volume reached 50 mm³. Each data point represents the mean value of 10 animals. Error bars represent standard deviation of the mean values.

FIG. 26 illustrates tumor growth kinetics of CT26 tumor-bearing BALB/c mice administrated intravenously with QW×2 dose of 3.75 μg/kg (open circle) mouse FLT3-Ligand mouse IgG2a (C136S, LALA-PG), Q3W dose of 3 mg/kg (open triangle) anti-mouse CTLA4 (clone 9D9), combination of the two reagents (closed circle) or Q3W dose of 10 mg/kg mouse IgG2a isotype control (open star) at day 0 when tumor volume reached 65 mm³. Each data point represents the mean value of 10 animals. Error bars represent standard deviation of the mean values.

FIG. 27 diagrams an immunogenicity study in C57BL/6 mice that were transduced with an Adeno-Associated Virus (AAV) vector encoding a 1.2× length hepatitis B virus (HBV) genome (AAV-HBV mice). At indicated times (asterisks) AAV-HBV mice were administered 3 doses of an HBV vaccine and treated with saline, mouse FLT3L, anti-mouse inhibitory PD-1, anti-mouse inhibitory CTLA-4 or anti-mouse stimulatory CD137 antibodies. A control group of mice received the HBV vaccine alone but no AAV-HBV. HBV-specific IFN-γ ELISPOT was performed using spleens of all animals at day 105 post first vaccination.

FIGS. 28A-C illustrate IFN-γ ELISPOT responses of AAV-HBV mice specific for HBsAg (FIG. 28A), HBV core (FIG. 28B) and HBV polymerase (FIG. 28C) observed at the end of the immunogenicity study diagrammed in FIG. 27 for indicated treatment and control groups.

FIG. 29A provides an overview of the study design for a Phase 1 Study in Healthy Volunteers to Evaluate the Single Dose Pharmacokinetics, Safety, and Tolerability of a FLT3L-Fc fusion protein comprising the amino acid sequence of SEQ ID NO: 14.

FIG. 29B provides a schematic of the timing of the PD assessments.

FIG. 29C illustrates concentration-time profiles of FLT3L-Fc fusion protein following single IV infusion administration of 75 μg (triangle), 225 μg (diamond), or 675 μg (circle) of FLT3L-Fc fusion proteins to healthy volunteers.

FIG. 29D illustrates comparisons of cDC1 cells quantitative changes over time in placebo (square with dashed lines), cohort 1 (+), cohort 2 (square with solid line), cohort 3 (triangle), and cohort 4 (circle).

FIG. 29E illustrates comparisons of cDC2 cell quantitative changes over time in placebo (square with dashed lines), cohort 1 (+), cohort 2 (square with solid line), cohort 3 (triangle), and cohort 4 (circle).

FIGS. 29F-G illustrate changes in circulating monocytes over time in placebo (square with dashed lines), cohort 1 (+), cohort 2 (square with solid line), cohort 3 (triangle), and cohort 4 (circle).

FIG. 30A provides an overview of the study design for Phase 1b Dose Escalation Study to Evaluate the Safety, Tolerability, Pharmacokinetics, and Preliminary Efficacy of a FLT3L-Fc fusion protein (SEQ ID NO: 14) in Subjects with Advanced Solid Tumors.

FIG. 30B provides a schematic of the 3+3 Dose Escalation Scheme.

FIG. 30C illustrates the schedule of intensive pharmacokinetic assessments for a Phase 1b Dose Escalation Study to Evaluate the Safety, Tolerability, Pharmacokinetics, and Preliminary Efficacy of a FLT3L-Fc fusion protein (SEQ ID NO: 14) in Subjects with Advanced Solid Tumors.

FIG. 30D provides a study procedures table.

FIG. 31 shows the concentration-time profiles of a FLT3L-Fc fusion protein (SEQ ID NO: 14) in subjects dosed with 2000 ug (circle), 6000 ug (square), or 12000 mg (triangle) of the FLT3L-Fc fusion protein.

FIGS. 32A-D show cDC1 and cDC2 cell counts for subjects dosed with 12 mg of a FLT3L-Fc fusion protein (SEQ ID NO: 14). FIG. 32A shows absolute cDC1 count. FIG. 32B shows % change from baseline (BL) for cDC1 cells. FIG. 32C shows absolute cDC2 count. FIG. 32D shows % change from baseline for cDC2 cells. For FIGS. 32A-D, subject 1 is represented with a circle, subject 2 is represented as a triangle, and subject 3 is represented as a square.

FIGS. 33A-D show % change from baseline for cDC1 and cDC2 cells in subjects dosed with 2, 6, and 12 mg of a FLT3L-Fc fusion protein (SEQ ID NO: 14). FIG. 33A shows % change from baseline for cDC1 cells in subjects dosed with 2 mg of the FLT3L-Fc fusion protein. FIG. 33B shows % change from baseline for cDC1 cells in subjects dosed with 6 mg of the FLT3L-Fc fusion protein. FIG. 33C shows % change from baseline for cDC2 cells in subjects dosed with 2 mg of the FLT3L-Fc fusion protein. FIG. 33D shows % change from baseline for cDC2 cells in subjects dosed with 6 mg of the FLT3L-Fc fusion protein. For FIGS. 33A-D, subject 1 is represented with a circle, subject 2 is represented as a triangle, and subject 3 is represented as a square.

### DETAILED DESCRIPTION

1. Introduction

Fms related tyrosine kinase 3 ligand (FLT3L) extracellular domain-immunoglobulin fragment crystallizable region (Fc region) fusion proteins with a prolonged serum half-life as compared to a wild-type Fc have been described in International Publication No. WO2020263830. The present disclosure provides dosing, dosing regimens and schedules, and combination therapies for administering the FLT3L-fusion proteins to a subject in need thereof. The dosing and dosing regimens and schedules are based on data from the first clinical trials conducted in human subjects, which evaluated the safety, efficacy, and tolerability of FLT3L-Fc fusion proteins. To date, there have been no studies or experimental data conducted in human subjects demonstrating the effective dose or dosing regimens and schedules for administering FLT3L-Fc fusion proteins to human subjects. Examples 31 and 32 of the present disclosure provide the first in human data for administration of FLT3L-Fc fusion proteins in human subjects, including healthy subjects and subjects having solid tumors.

2. FLT3L-Fc Fusion Proteins and Homodimers Thereof

Provided are fusion proteins comprising a fms related tyrosine kinase 3 ligand (FLT3L) extracellular domain operably linked to an immunoglobulin fragment crystallizable region (Fc region), wherein at least 5 amino acids are truncated from the C-terminus of the FLT3L extracellular domain; and/or wherein the Fc region does not comprise a hinge region.

In some embodiments, the FLT3L fusion proteins provided herein are capable of binding to human fms related tyrosine kinase 3 ligand (FLT3). Human fms related tyrosine kinase 3 is identified as NCBI Gene ID 2322 and is also known as human CD135, FLK-2, FLK2, or STK1. Binding of FLT3L fusion proteins to FLT3L can be analyzed, for example, by FACS, SPR, ELISA, immunoprecipitation-western blot, and other assay formats known in the art.

A. FLT3L Extracellular Domain

In certain embodiments, the FLT3L extracellular domain comprises or is derived from a human FLT3L sequence. Human fms related tyrosine kinase 3 ligand is identified as NCBI Gene ID 2323 and the alternative symbols of FLT3LG, FLT3L, FL and FLG3L. NCBI identifies two isoforms and five transcriptional variants. Exemplary polynucleotide and polypeptide sequences of FLT3L include Ref Seq Nos. NM_001204502.1→NP_001191431.1 (isoform 1, transcript variant 1); NM_001204503.1→NP_001191432.1 (isoform 1, transcript variant 2); NM_001459.4→NP_001450.2 (isoform 1, transcript variant 3); NM_001278637.1→NP_001265566.1 (isoform 2, transcript variant 4); and NM_001278638.1→NP_001265567.1 (isoform 2, transcript variant 5). In some embodiments, the FLT3L extracellular domain comprises an amino acid sequence having at least 80%, at least 85%, at least 90%, at least 91%, at least 92%, at least 93%, at least 94%, at least 95%, at least 96%, at least 97%, at least 98%, at least 99%, or at least 100% identical to an amino acid sequence of NP_001191431.1, NP_001191432.1, NP_001450.2, NP_001265566.1 or NP_001265567.1, wherein the FLT3L extracellular domain binds to and activates signaling through fms related tyrosine kinase 3 (FLT3, CD135, FLK2, STK1). In some embodiments, the FLT3L extracellular domain comprises or is derived from human FLT3L isoform 1. In some embodiments, the FLT3L extracellular domain comprises or is derived from human FLT3L isoform 2.

In some embodiments, the FLT3L portion of the fusion protein comprises an amino acid sequence having at least 80%, at least 85%, at least 90%, at least 91%, at least 92%, at least 93%, at least 94%, at least 95%, at least 96%, at least 97%, at least 98%, at least 99%, or at least 100% identical to an amino acid sequence of

(SEQ ID NO: 71)
TQDCSFQHSPISSDFAVKIRELSDYLLQDYPVTVASNLQDEELCGGLWRLV
LAQRWMERLKTVAGSKMQGLLERVNTEIHFVTKCAFQPPPSCLRFVQTNIS
RLLQETSEQLVALKPWITRQNFSRCLELQCQPDSSTLPPPWSPRP;
(SEQ ID NO: 72)
TQDCSFQHSPISSDFAVKIRELSDYLLQDYPVTVASNLQDEELCGGLWRLV
LAQRWMERLKTVAGSKMQGLLERVNTEIHFVTKCAFQPPPSCLRFVQTNIS
RLLQETSEQLVALKPWITRQNFSRCLELQCQPDSSTLPPPWSPRPL;
(SEQ ID NO: 73)
TQDCSFQHSPISSDFAVKIRELSDYLLQDYPVTVASNLQDEELCGGLWRLV
LAQRWMERLKTVAGSKMQGLLERVNTEIHFVTKCAFQPPPSCLRFVQTNIS
RLLQETSEQLVALKPWITRQNFSRCLELQCQPDSSTLPPPWSPRPLE;
(SEQ ID NO: 74)
TQDCSFQHSPISSDFAVKIRELSDYLLQDYPVTVASNLQDEELCGGLWRLV
LAQRWMERLKTVAGSKMQGLLERVNTEIHFVTKCAFQPPPSCLRFVQTNIS
RLLQETSEQLVALKPWITRQNFSRCLELQCQPDSSTLPPPWSPRPLEA;
(SEQ ID NO: 75)
TQDCSFQHSPISSDFAVKIRELSDYLLQDYPVTVASNLQDEELCGGLWRLV
LAQRWMERLKTVAGSKMQGLLERVNTEIHFVTKCAFQPPPSCLRFVQTNIS
RLLQETSEQLVALKPWITRQNFSRCLELQCQPDSSTLPPPWSPRPLEAT;
(SEQ ID NO: 76)
TQDCSFQHSPISSDFAVKIRELSDYLLQDYPVTVASNLQDEELCGGLWRLV
LAQRWMERLKTVAGSKMQGLLERVNTEIHFVTKCAFQPPPSCLRFVQTNIS
RLLQETSEQLVALKPWITRQNFSRCLELQCQPDSSTLPPPWSPRPLEATA;
(SEQ ID NO: 77)
TQDCSFQHSPISSDFAVKIRELSDYLLQDYPVTVASNLQDEELCGGLWRLV
LAQRWMERLKTVAGSKMQGLLERVNTEIHFVTKCAFQPPPSCLRFVQTNIS
RLLQETSEQLVALKPWITRQNFSRCLELQCQPDSSTLPPPWSPRPLEATA
P;
(SEQ ID NO: 78)
TQDCSFQHSPISSDFAVKIRELSDYLLQDYPVTVASNLQDEELCGGLWRLV
LAQRWMERLKTVAGSKMQGLLERVNTEIHFVTKCAFQPPPSCLRFVQTNIS
RLLQETSEQLVALKPWITRQNFSRCLELQCQPDSSTLPPPWSPRPLEATAP
T;
(SEQ ID NO: 79)
TQDCSFQHSPISSDFAVKIRELSDYLLQDYPVTVASNLQDEELCGGLWRLV
LAQRWMERLKTVAGSKMQGLLERVNTEIHFVTKCAFQPPPSCLRFVQTNIS
RLLQETSEQLVALKPWITRQNFSRCLELQCQPDSSTLPPPWSPRPLEATAP
TA;
(SEQ ID NO: 80)
TQDCSFQHSPISSDFAVKIRELSDYLLQDYPVTVASNLQDEELCGGLWRLV
LAQRWMERLKTVAGSKMQGLLERVNTEIHFVTKCAFQPPPSCLRFVQTNIS
RLLQETSEQLVALKPWITRQNFSRCLELQCQPDSSTLPPPWSPRPLEATAP
TAP;
or
(SEQ ID NO: 81)
TQDCSFQHSPISSDFAVKIRELSDYLLQDYPVTVASNLQDEELCGGLWRLV
LAQRWMERLKTVAGSKMQGLLERVNTEIHFVTKCAFQPPPSCLRFVQTNIS
RLLQETSEQLVALKPWITRQNFSRCLELQCQPDSSTLPPPWSPRPLEATAP
TAPQ;

wherein the FLT3L extracellular domain binds to and activates signaling through fms related tyrosine kinase 3(Gene ED: 2322; FLT3, CD135, FLK2, STK1), and promotes or increases the proliferation of cells expressing FLT3 on their cell surface. In some embodiments, one or more FLT3L domain amino acid residues N100, 5102, N123 and 5125 are substituted, e.g., to remove the N-X-S/T motifs, and potential N-linked and/or O-linked glycosylation sites, e.g., to an amino acid residue selected from the group consisting of glycine (G), alanine (A), or valine (V), wherein the amino acid residue positions are with reference to SEQ ID NOs: 1-18, 21-27 or 71-81. In some embodiments, one or both of the serine residues at positions 102 and 125 are substituted to alanine (A), wherein the amino acid residue positions are with reference to SEQ ID NOs: 1-18, 21-27 or 71-81. In some embodiments, the FLT3L extracellular domain comprises one or more amino acid substitutions at the following positions: H8, K84, S102 and/or S125, wherein the amino acid residue positions are with reference to SEQ ID NOs: 1-18, 21-27 or 71-81. In some embodiments, the FLT3L extracellular domain comprises one or more of the following amino acid substitutions: H8Y, K84E; S102A; and/or S125A; wherein the amino acid residue positions are with reference to SEQ ID NOs: 1-18, 21-27 or 71-81.

Modifications may be made in the structure of the FLT3L-Fc polynucleotides and polypeptides described herein and still obtain a functional molecule that encodes a variant or derivative polypeptide with desirable characteristics. When it is desired to alter the amino acid sequence of a polypeptide to create an equivalent, or even an improved, variant or portion of a polypeptide described herein, one skilled in the art will typically change one or more of the codons of the encoding DNA sequence.

For example, certain amino acids may be substituted for other amino acids in a protein structure without appreciable loss of its ability to bind other polypeptides (e.g., antigens) or cells. Since it is the binding capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the polypeptide sequences of the disclosed antibodies and antigen-binding fragments thereof, or corresponding DNA sequences that encode said polypeptides without appreciable loss of their biological utility or activity.

In many instances, a polypeptide variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, or over the full length of a sequence, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5: 151-153; Myers, E. W. and Muller W. (1988) CABIOS 4:11-17; Robinson, E. D. (1971) Comb. Theor 77: 105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides described herein. Software for performing BLAST analyses is publicly available through the National Center for Biotechnology Information (blast.ncbi.nlm.nih.gov/Blast.cgi).

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a word length (W) of 11, and expectation (E) of 10, and the BLOSUM62 scoring matrix (see Henikoff and Henikoff (1989) Proc. Natl. Acad. Sci. USA 89: 10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

In some embodiments, the FLT3L extracellular domain does not comprise a signal peptide. In some embodiments, the FLT3L extracellular domain comprises an N-terminal signal peptide. The signal peptide can be an endogenous signal peptide (e.g., from a native or wild-type FLT3L protein), or from a heterologous polypeptide. In some embodiments, the heterologous signal peptide is from a secreted protein, e.g., a serum protein, an immunoglobulin or a cytokine. In some embodiments, the signal peptide is from a serum albumin signal peptide (e.g., having the amino acid sequence KWVTFISLLFLFSSAYS (SEQ ID NO:82). In some embodiments, the signal peptide is from a FLT3L protein (e.g., having the amino acid sequence MTVLAPAWSPTTYLLLLLLLSSGLSG (SEQ ID NO:83) or MTVLAPAWSPNSSLLLLLLLLSPCLRG (SEQ ID NO:84). The signal peptide can be designed to be cleaved off, e.g., after secretion from the cell, to form a mature fusion protein. A modified human serum albumin signal peptide to secrete proteins in cells that can find use in expressing the present fusion proteins is described, e.g., in Attallah, et al., Protein Expr Purif. (2017) 132:27-33. Additional signal peptide sequences for use in expressing the herein described fusion proteins are described, e.g., in Kober, et al., Biotechnol Bioeng. (2013) 110(4):1164-73.

In some embodiments, at least five amino acids are truncated from the C-terminus of the FLT3L extracellular domain. For example, in various embodiments, at least 5, 6, 7, 8, 9, 10, 11, 12, 13, 14 or 15 amino acid residues are truncated or removed from the C-terminus of the FLT3L extracellular domain. In some embodiments, the FLT3L extracellular domain in the fusion protein is no longer than 147, 148, 149, 150, 151, 152, 153, 154, 155, 156 or 157 amino acid residues in length. In some embodiments, the FLT3L extracellular domain does not comprise the amino acid sequence PTAPQ (SEQ ID NO:85), APTAPQ (SEQ ID NO:86), TAPTAPQ (SEQ ID NO:87), ATAPTAPQ (SEQ ID NO:88), EATAPTAPQ (SEQ ID NO:89), or LEATAPTAPQ (SEQ ID NO:90). In some embodiments, the FLT3L extracellular domain does not comprise the amino acid sequence PTAPQPP (SEQ ID NO:91), APTAPQPP (SEQ ID NO:92), TAPTAPQPP (SEQ ID NO:93), ATAPTAPQPP (SEQ ID NO:94), EATAPTAPQPP (SEQ ID NO:95), or LEATAPTAPQPP (SEQ ID NO:96).

In certain embodiments, the FLT3L extracellular domain comprises or is derived from a mouse or murine FLT3L sequence. Mus musculus fms related tyrosine kinase 3 ligand is identified as NCBI Gene ID 14256 and the alternative symbols of Flt31, Ly72L and Flt31g. NCBI identifies one validated isoform and three unvalidated isoforms (X1, X2 and X3). Exemplary polynucleotide and polypeptide sequences of FLT3L include RefSeq Nos. NM_013520.3→NP_038548.3 (validated isoform 1); XM_006540607.3→XP_006540670.1 (isoform X1); XM_006540608.3→XP_006540671.1 (isoform X1); XM_006540606.2→XP_006540669.1 (isoform X1); XM_011250793.1→XP_011249095.1 (isoform X1); XM_006540609.3→XP_006540672.1 (isoform X2); XM_006540610.3→XP_006540673.1 (isoform X2); XM_006540612.3→XP_006540675.1 (isoform X3); and XM_011250794.2→XP_011249096.1 (isoform X3). In some embodiments, the FLT3L extracellular domain comprises an amino acid sequence having at least 80%, at least 85%, at least 90%, at least 91%, at least 92%, at least 93%, at least 94%, at least 95%, at least 96%, at least 97%, at least 98%, at least 99%, or at least 100% identical to an amino acid sequence of NP_038548.3, XP_006540670.1, XP_006540671.1, XP_006540669.1, XP_011249095.1, XP_006540672.1, XP_006540673.1, XP_006540675.1, XP_011249096.1, wherein the FLT3L extracellular domain binds to and activates signaling through fms related tyrosine kinase 3 (FLT3, CD135, FLK2, STK1), and promotes or increases the proliferation of cells expressing FLT3 on their cell surface. In some embodiments, the FLT3L extracellular domain comprises or is derived from murine FLT3L isoforms 1, X1, X2 or X3. In some embodiments, at least five amino acids are truncated from the C-terminus of the mouse FLT3L extracellular domain. For example, in various embodiments, at least 5, 6, 7, 8, 9, 10, 11, 12, 13, 14 or 15 amino acid residues are truncated or removed from the C-terminus of the mouse FLT3L extracellular domain. In some embodiments, the FLT3L extracellular domain in the fusion protein is no longer than 149, 150, 151, 152, 153, 154, 155, 156, 157, 158 or 159 amino acid residues in length.

In some embodiments, the mouse FLT3L portion of the fusion protein comprises an amino acid sequence having at least 80%, at least 85%, at least 90%, at least 91%, at least 92%, at least 93%, at least 94%, at least 95%, at least 96%, at least 97%, at least 98%, at least 99%, or at least 100% identical to an amino acid sequence of: TPDCYFSHSPISSNFKVKFRELTDHLLKDYPVTVAVNLQDEKHCKALWSLFLAQRWIEQ LKTVAGSKMQTLLEDVNTEIHFVTSCTFQPLPECLRFVQTNISHLLKDTCTQLLALKPCI GKACQNFSRCLEVQCQPDSSTLLPPRSPIALEATELPEPR (SEQ ID NO:98), wherein the mouse FLT3L extracellular domain binds to and activates signaling through mouse fms related tyrosine kinase 3 (NCBI Human Gene ID: 14255; Flt3, Flk2; Ly72; wmfl; CD135; Flk-2; Flt-3; B230315G04). In some embodiments, cysteine at position 109 is substituted to an amino acid residue selected from the group consisting of glycine (G), alanine (A), serine (S), threonine (T) or valine (V), wherein the amino acid residue positions are with reference to SEQ ID NOs: 19, 20 and 42.

In certain embodiments, the FLT3L extracellular domain comprises or is derived from a macaque or macaca FLT3L sequence. Macaca mulatta (Rhesus monkey) fms related tyrosine kinase 3 ligand is identified as NCBI Gene ID 719239 and the alternative symbols of FLT3L and FLT3LG. NCBI identifies five unvalidated isoforms (X1, X2, X3, X4, X5). Exemplary polynucleotide and polypeptide sequences of FLT3L include RefSeq Nos. XM_015124576.1→XP_014980062.1 (isoform X1), XM_015124578.1→XP_014980064.1 (isoform X2), XM_015124579.1→XP_014980065.1 (isoform X3), XM_015124580.1→XP_014980066.1 (isoform X4) and XM_015124581.1→XP_014980067.1 (isoform X5). In some embodiments, the FLT3L extracellular domain comprises an amino acid sequence having at least 80%, at least 85%, at least 90%, at least 91%, at least 92%, at least 93%, at least 94%, at least 95%, at least 96%, at least 97%, at least 98%, at least 99%, or at least 100% identical to an amino acid sequence of XP_014980062.1, XP_014980064.1, XP_014980065.1, XP_014980066.1 or XP_014980067.1, wherein the FLT3L extracellular domain binds to and activates signaling through fms related tyrosine kinase 3 (FLT3, CD135, FLK2, STK1), and promotes or increases the proliferation of cells expressing FLT3 on their cell surface. In some embodiments, the FLT3L extracellular domain comprises or is derived from macaque FLT3L isoforms X1, X2, X3, X4 or X5. In some embodiments, at least five amino acids are truncated from the C-terminus of the macaque FLT3L extracellular domain. For example, in various embodiments, at least 5, 6, 7, 8, 9, 10, 11, 12, 13, 14 or 15 amino acid residues are truncated or removed from the C-terminus of the macaque FLT3L extracellular domain. In some embodiments, the FLT3L extracellular domain in the fusion protein is no longer than 145, 146, 147, 148, 149, 150, 151, 152, 153, 154, 155, 156, 157, 158, 159, 160, 161, 162, 163, 164 or 165 amino acid residues in length.

As appropriate, in certain embodiments, the FLT3L extracellular domain is comprised of or derived from a canine or a feline FLT3L extracellular domain. In some embodiments, the dog or Canis lupus FLT3L portion of the fusion protein comprises an amino acid sequence having at least 80%, at least 85%, at least 90%, at least 91%, at least 92%, at least 93%, at least 94%, at least 95%, at least 96%, at least 97%, at least 98%, at least 99%, or at least 100% identical to an amino acid sequence of: NP_001003350.1, XP_005615795.1 or XP_0222731641 In some embodiments, the cat or Felis catus FLT3L portion of the fusion protein comprises an amino acid sequence having at least 80%, at least 85%, at least 90%, at least 91%, at least 92%, at least 93%, at least 94%, at least 95%, at least 96%, at least 97%, at least 98%, at least 99%, or at least 100% identical to an amino acid sequence of: NP_001009842.1 or XP_011287950.1.

A “polypeptide variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences described herein and evaluating one or more biological activities of the polypeptide as described herein and/or using any of a number of techniques well known in the art.

The term “variant” may also refer to any naturally occurring or engineered molecule comprising one or more nucleotide or amino acid mutations. In one embodiment, the multi-specific antigen binding molecule is a bispecific antigen binding molecule. In one embodiment, the multi-specific antigen binding molecule is a bispecific antibody. For example, somatic variants may encompass all related naturally occurring antibodies that are part of or derived from the same B-cell lineage. Engineered variants may encompass all single mutations or combinatorial mutations made to an antibody.

B. Fc Region

The FLT3L extracellular domain, or truncated fragment thereof, is operably linked to an Fc domain. Generally, the Fc domain is comprised of or derived from the same species as the FLT3L extracellular domain (e.g., human, dog, cat, mouse or monkey). In some embodiments, the FLT3L extracellular domain, or truncated fragment thereof, is directly linked or contiguously linked or abutted to the Fc domain. In some embodiments, the FLT3L extracellular domain, or truncated fragment thereof, is operably linked to the Fc domain via a linker. As appropriate, the linker can be a flexible linker, e.g., a sequence comprising 3 or 4 repeats of a GGGGS motif or “G-S linker” (SEQ ID NO:99) (Desplancq et al. 1994, Protein Engineering 7:1027-1033).

In some embodiments, the Fc region is from a human IgG1, IgG2, IgG3 or IgG4. In some embodiments, the Fc region is from a human IgG1 or IgG4.

In certain embodiments the FLT3L extracellular domain, or truncated fragment thereof, is directly linked to, or linked via an intervening amino acid sequence (e.g., a G-S linker), to a human IgG1 (e.g., mutant IgG1m3 sequence), IgG2, IgG3 or IgG4 with 1 to 10 (e.g., 1, 2, 3, 4, 5, 6, 7, 8, 9, 10) amino acid substitutions. In some embodiments, the Fc modifications can promote one or more of increased serum half-life or decreased antibody effector function of the molecule. In other embodiments, certain of these modifications, decrease antibody effector function and increase half-life of the antibody. In some embodiments, the FLT3L-Fc fusion proteins described herein comprise two or more, three or more, four or more, five or more, six or more, six or fewer, five or fewer, four or fewer, three or fewer, two or fewer, or one modified Fc amino acid residue(s). Exemplary amino acid substitutions are described below.

In some embodiments, the Fc domain of the fusion protein does not comprise a hinge region; it is truncated or deleted, in whole or in part. The structural hinge region of human IgG1, IgG2 and IgG4 antibodies is a peptide linker of about 19 to 23 amino acids containing two to four cysteine residues, is genetically encoded on the hinge exon together with the 5′-end of the CH2 exon, and allows for disulfide bridges between first and second Fc domains (Roux, et al., J. Immunol. (1998) 161:4083). The structural hinge region is comprised of amino acid residue positions 216-238 (EU numbering) or 226-251 (Kabat numbering) (identified on imgt.org). In some embodiments, the Fc region comprises or is derived from a human IgG4 isotype and does not comprise the amino acid sequence ESKYGPPCPPCP (SEQ ID NO:100). In some embodiments, the Fc region comprises or is derived from a human IgG1 isotype and does not comprise the amino acid sequence EPKSCDKTHTCPPCP (SEQ ID NO:101) or EPKSCDKTHTCPPCPAPELL (SEQ ID NO:110).

Fc Mutations that Increase Serum Half-Life

In some embodiments, the Fc region comprises amino acid modifications that promote an increased serum half-life of the fusion protein. Mutations that increase the half-life of an antibody have been described. In one embodiment, the constant region of a FLT3L-Fc fusion proteins described herein comprise a methionine to tyrosine substitution at position 252 (EU numbering), a serine to threonine substitution at position 254 (EU numbering), and a threonine to glutamic acid substitution at position 256 (EU numbering). See, e.g., U.S. Pat. No. 7,658,921. This type of mutant, designated as a “YTE mutant” exhibits a four-fold increased half-life relative to wild-type versions of the same antibody (Dall'Acqua, et al., J Biol Chem, 281: 23514-24 (2006); Robbie, et al., Antimicrob Agents Chemotherap., 57(12):6147-6153 (2013)). In certain embodiments, the FLT3L-Fc fusion proteins described herein comprise an IgG constant domain comprising one, two, three or more amino acid substitutions of amino acid residues at positions 251-257, 285-290, 308-314, 385-389, and 428-436 (EU numbering). Alternatively, M428L and N434S (“LS”) substitutions can increase the pharmacokinetic half-life of the fusion protein. In other embodiments, the FLT3L-Fc fusion proteins described herein comprise a M428L and N434S substitution (EU numbering). In other embodiments, the FLT3L-Fc fusion proteins described herein comprise T250Q and M428L (EU numbering) mutations. In other embodiments, the FLT3L-Fc fusion proteins described herein comprise H433K and N434F (EU numbering) mutations.

Fc Mutations that Reduce or Eliminate Effector Activity

In some embodiments, the FLT3L-Fc fusion proteins described herein can have an Fc domain with amino acid substitutions that reduce or eliminate Fc effector function (including, e.g., antibody-dependent cellular cytotoxicity (ADCC), antibody-dependent cellular phagocytosis (ADCP), and complement-dependent cytotoxicity (CDC)).

In some embodiments, the Fc region is altered by replacing at least one amino acid residue with a different amino acid residue to reduce or eliminate effector function(s) of the antibody. For example, one or more amino acids selected from amino acid residues 234, 235, 236, 237, 297, 318, 320 and 322 (EU numbering) can be replaced with a different amino acid residue such that the fusion protein has decreased affinity for an effector ligand. The effector ligand to which affinity is altered can be, for example, an Fc receptor (e.g., at residue positions 234, 235, 236, 237, 297 (EU numbering)) or the C1 component of complement (e.g., at residue positions 297, 318, 320, 322 (EU numbering)). U.S. Pat. Nos. 5,624,821 and 5,648,260, both by Winter et al.

Fc modifications reducing or eliminating effector function include substitutions, insertions, and deletions, e.g., at one or more positions including 234, 235, 236, 237, 267, 269, 325, and 328, e.g., 234G, 235G, 236R, 237K, 267R, 269R, 325L, and 328R (EU numbering). Further, an Fc variant may comprise 236R/328R. Other modifications for reducing FcγR and complement interactions include substitutions at positions 297A, 234A, 235A, 318A, 228P, 236E, 268Q, 309L, 330S, 3315, 220S, 226S, 229S, 238S, 233P, and 234V (EU numbering). These and other modifications are reviewed in Strohl (2009) Current Opinion in Biotechnology 20:685-691. Effector functions (both ADCC and complement activation) can be reduced, while maintaining neonatal FcR binding (maintaining half-life), by mutating IgG residues at one or more of positions 233-236 and 327-331, such as E233P, L234V, L235A, optionally G236A, A327G, A330S and P331S in IgG1; E233P, F234V, L235A, optionally G236A, in IgG4; and A330S and P331S in IgG2 (EU numbering). See Armour et al. (1999) Eur. J. Immunol. 29:2613; WO 99/58572. Other mutations that reduce effector function include L234A and L235A in IgG1 (Alegre et al. (1994) Transplantation 57:1537); V234A and G237A in IgG2 (Cole et al. (1997) J. Immunol. 159:3613; see also U.S. Pat. No. 5,834,597); and S228P and L235E for IgG4 (Reddy et al. (2000) J. Immunol. 164:1925). Another combination of mutations for reducing effector function in a human IgG1 include L234F, L235E and P331S. Oganesyan et al. (2008) Acta Crystallogr. D. Biol. Crystallogr. 64:700. See generally Labrijn et gal. (2008) Curr. Op. Immunol. 20:479. Additional mutations found to decrease effector function in the context of an Fc (IgG1) fusion protein (abatacept) include C226S, C229S and P238S (EU numbering). Davis et al. (2007) J. Immunol. 34:2204.

ADCC activity may be reduced by modifying the Fc region. In certain embodiments, sites that affect binding to Fc receptors may be removed, e.g., sites other than salvage receptor binding sites. In other embodiments, an Fc region may be modified to remove an ADCC site. Exemplary ADCC sites have been described with respect to ADCC sites in IgG1 (Sarmay, et al, (1992) Molec. Immunol. 29 (5): 633-9). In one embodiment, the G236R and L328R variant of human IgG1 effectively eliminates FcγR binding (Horton, et al. (2011) J. Immunol. 186:4223 and Chu, et al. (2008) Mol. Immunol. 45:3926). In other embodiments, the Fc having reduced binding to FcγRs comprises the amino acid substitutions L234A, L235E and G237A. Gross, et al. (2001) Immunity 15:289. Modifications in the IgG Fc region to decrease binding to FcγRI to decrease ADCC (e.g., 234A; 235E; 236A; G237A) identified in WO 88/007089 can be used in the present fusion proteins. See also Duncan & Winter (1988) Nature 332:563; Chappel et al. (1991) Proc. Nat'l Acad. Sci. (USA) 88:9036; and Sondermann et al. (2000) Nature 406:267 (discussing the effects of these mutations on FcγRIII binding).

CDC activity may also be reduced by modifying the Fc region. Mutations at IgG1 positions D270, K322, P329 and P331, specifically alanine mutations D270A, K322A, P329A and P331A, significantly reduce the ability of the corresponding antibody to bind C1q and activate complement (Idusogie et al. (2000) J. Immunol. 164:4178; WO 99/51642. Modification of position 331 of IgG1 (e.g., P331S) has been shown to reduce complement binding (Tao et al. (1993) J. Exp. Med. 178:661; Xu Y, et al. J Biol Chem. 1994. 269:3469-74; and Canfield & Morrison (1991) J. Exp. Med. 173:1483). In another example, one or more amino acid residues within amino acid positions 231 to 239 are altered to thereby reduce the ability of the antibody to fix complement (WO 94/29351). Modifications in the IgG Fc region identified in WO 88/007089 that reduce or eliminate binding to complement component C1q, and therefore reduce or eliminate CDC (e.g., E318A or V/K320A and K322A/Q) can be used in the present fusion proteins.

In some embodiments, the Fc with reduced complement fixation has the amino acid substitutions A330S and P331S. Gross et al. (2001) Immunity 15:289.

Other Fc variants having reduced ADCC and/or CDC are disclosed at Glaesner et al. (2010) Diabetes Metab. Res. Rev. 26:287 (F234A and L235A to decrease ADCC and ADCP in an IgG4); Hutchins et al. (1995) Proc. Nat'l Acad. Sci. (USA) 92:11980 (F234A, G237A and E318A in an IgG4); An et al. (2009) MAbs 1:572 and U.S. Pat. App. Pub. 2007/0148167 (H268Q, V309L, A330S and P331S in an IgG2); McEarchern et al. (2007) Blood 109:1185 (C226S, C229S, E233P, L234V, L235A in an IgG1); Vafa et al. (2014) Methods 65:114 (V234A, G237A, P238S, H268A, V309L, A330S, P331S in an IgG2) (EU numbering).

In certain embodiments, the fusion protein has an Fc having essentially no effector function, e.g., the Fc has reduced or eliminated binding to FcγRs and reduced or eliminated complement fixation, e.g., is effectorless. An exemplary IgG1 Fc that is effectorless comprises the following five mutations: L234A, L235E, G237A, A330S and P331S (EU numbering) (Gross et al. (2001) Immunity 15:289). These five substitutions may be combined with N297A to eliminate glycosylation as well.

### IgG1 Isotype Fc

In one embodiment, the Fc region comprises or is derived from a human IgG1. In some embodiments, the antibody has a chimeric heavy chain constant region (e.g., having the CH1, hinge, CH2 regions of IgG4 and CH3 region of IgG1).

IgG1 antibodies exist in various allotypes and isoallotypes. In particular embodiments, the FLT3L-Fc fusion proteins described herein include an IgG1 heavy chain having an allotype of G1m1; nG1m2; G1m3; G1m17,1; G1m17,1,2; G1m3,1; or G1m17. Each of these allotypes or isoallotypes is characterized by the following amino acid residues at the indicated positions within the IgG1 heavy chain constant region (Fc) (EU numbering):

G1m1: D356, L358;

nG1m1: E356, M358;

G1m3: R214, E356, M358, A431;

G1m17,1: K214, D356, L358, A431;

G1m17,1,2: K214, D356, L358, G431;

G1m3,1: R214, D356, L358, A431; and

G1m17: K214, E356, M358, A431.

In a specific embodiment, the FLT3L extracellular domain, or truncated fragment thereof, is directly linked to, or linked via an intervening amino acid sequence (e.g., a G-S linker), to a wild type IgG1m3 sequence, or fragment thereof, provided below.

(SEQ ID NO: 102)
EPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQ
GNVFSCSVMHEALHNHYTQKSLSLSPGK.

In certain embodiments, the FLT3L-Fc fusion protein has an IgG1 isotype. In some embodiments, the FLT3L-Fc fusion protein contains a human IgG1 constant region. In some embodiments, the human IgG1 Fc region contains one or more modifications. For example, in some embodiments, the Fc region contains one or more amino acid substitutions (e.g., relative to a wild-type Fc region of the same isotype). In some embodiments, the one or more amino acid substitutions are selected from N297A, N297Q (Bolt S et al. (1993) Eur J Immunol 23:403-411), D265A, L234A, L235A (McEarchem et al., (2007) Blood, 109:1185-1192), C226S, C229S (McEarchem et al., (2007) Blood. 109:1185-1192), P238S (Davis et al., (2007) J Rheumatol, 34:2204-2210), E233P, L234V (McEarchern et al., (2007) Blood, 109:1185-1192), P238A, A327Q, A327G, P329A (Shields R L. et al., (2001) J Biol Chem. 276(9):6591-604), K322A, L234F, L235E (Hezareh, et al., (2001) J Virol 75, 12161-12168; Oganesyan et al., (2008). Acta Crystallographica 64, 700-704), P331S (Oganesyan et al., (2008) Acta Crystallographica 64, 700-704), T394D (Wilkinson et al. (2013) MAbs 5(3): 406-417), A330L, M252Y, S254T, and/or T256E, where the amino acid position is according to the EU numbering convention. In certain embodiments, the Fc region further includes an amino acid deletion at a position corresponding to glycine 236 according to the EU numbering convention. As used herein, numbering of a given amino acid polymer or nucleic acid polymer “corresponds to”, is “corresponding to” or is “relative to” the numbering of a selected or reference amino acid polymer or nucleic acid polymer when the position of any given polymer component (e.g., amino acid, nucleotide, also referred to generically as a “residue”) is designated by reference to the same or to an equivalent position (e.g., based on an optimal alignment or a consensus sequence) in the selected amino acid or nucleic acid polymer, rather than by the actual numerical position of the component in the given polymer.

In some embodiments, the FLT3L-Fc fusion protein has an IgG1 isotype with a heavy chain constant region that contains a C220S amino acid substitution according to the EU numbering convention.

In some embodiments, the Fc region comprises a human IgG1 isotype and comprises one or more amino acid substitutions in the Fc region at a residue position selected from the group consisting of: N297A, N297G, N297Q, N297G, D265A, L234A, L235A, C226S, C229S, P238S, E233P, L234V, P238A, A327Q, A327G, P329A, P329G, K322A, L234F, L235E, P331S, T394D, A330L, M252Y, S254T, T256E, M428L, N434S, T366W, T366S, L368A, Y407V and any combination thereof, wherein the numbering of the residues is according to EU numbering. In some embodiments, the Fc region comprises a human IgG1 isotype and comprises one or more amino acid substitutions in the Fc region at a residue position selected from the group consisting of: L234A, L234V, L234F, L235A, L235E, A330L, P331S, and any combination thereof, wherein the numbering of the residues is according to EU numbering.

### IgG4 Isotype Fc

For uses where effector function is to be avoided altogether, e.g., when antigen binding alone is sufficient to generate the desired therapeutic benefit, and effector function only leads to (or increases the risk of) undesired side effects, IgG4 antibodies may be used, or antibodies or fragments lacking the Fc region or a substantial portion thereof can be devised, or the Fc may be mutated to eliminate glycosylation altogether (e.g., N297A). Alternatively, a hybrid construct of human IgG2 (CH1 domain and hinge region) and human IgG4 (CH2 and CH3 domains) has been generated that is devoid of effector function, lacking the ability to bind the FcγRs (like IgG2) and unable to activate complement (like IgG4). (see, Rother et al. (2007) Nat. Biotechnol. 25:1256; Mueller et al. (1997) Mol. Immunol. 34:441; and Labrijn et al. (2008) Curr. Op. Immunol. 20:479, discussing Fc modifications to reduce effector function generally).

In one embodiment, the Fc region comprises or is derived from a human IgG4. In certain embodiments, the FLT3L-Fc fusion protein has an IgG4 isotype. In some embodiments, the FLT3L-Fc fusion protein contains a human IgG4 constant region. In some embodiments, the human IgG4 constant region includes an Fc region. In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from E233P, F234V, F234A, L235A, G237A, E318A, S228P, L235E, T394D, M252Y, S254T, T256E, N297A, N297G, N297Q, T366W, T366S, L368A, Y407V, M428L, N434S, and any combination thereof, where the amino acid position is according to the EU numbering convention. See, e.g., Hutchins et al. (1995) Proc Natl Acad Sci USA, 92:11980-11984; Reddy et al., (2000)J Immunol, 164:1925-1933; Angal et al., (1993) Mol Immunol. 30(1):105-8; U.S. Pat. No. 8,614,299 B2; Vafa O. et al., (2014) Methods 65:114-126; and Jacobsen et. al., J. Biol. Chem. (2017) 292(5):1865-1875. In some embodiments, the Fc region comprises a human IgG4 isotype and comprises one or more amino acid substitutions in the Fc region at a residue position selected from the group consisting of: F234V, F234A, L235A, L235E, S228P, and any combination thereof, wherein the numbering of the residues is according to EU numbering.

In some embodiments, an IgG4 variant of the present disclosure may be combined with an S228P mutation according to the EU numbering convention (Angal et al., (1993) Mol Immunol, 30:105-108) and/or with one or more mutations described in Peters et al., (2012) J Biol Chem. 13; 287(29):24525-33) to enhance antibody stabilization.

### IgG2 Isotype Fc

In certain embodiments, the FLT3L-Fc fusion protein has an IgG2 isotype. In some embodiments, the FLT3L-Fc fusion protein contains a human IgG2 constant region. In some embodiments, the human IgG2 constant region includes an Fc region. In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from P238S, V234A, G237A, H268A, H268Q, H268E, V309L, N297A, N297G, N297Q, V309L, A330S, P331 S, C232S, C233S, M252Y, S254T, and/or T256E, where the amino acid position is according to the EU numbering convention (Vafa, et al., (2014) Methods 65:114-126).

In certain embodiments, the FLT3L-Fc fusion proteins described herein comprise the L234F, L235E, D265A mutations, which are collectively referred to as “FEA.” The FEA mutations decrease or abrogate effector function. In certain embodiments, the FLT3L-Fc fusion proteins described herein comprise the L234F, L235E, D265A, and F405L mutations, which are collectively referred to as “FEAL.” In certain embodiments, the FLT3L-Fc fusion proteins described herein comprise the L234F, L235E, D265A, and a mutation selected from the group consisting of F405L, F405A, F405D, F405E, F405H, F4051, F405K, F405M, F405N, F405Q, F4055, F405T, F405V, F405W, and F405Y. In certain embodiments, the FLT3L-Fc fusion proteins described herein comprise the L234F, L235E, D265A, and K409R mutations, which are collectively referred to as “FEAR.” In certain embodiments, FEAL and FEAR are comprised in a fusion protein described herein. In certain embodiments, the FLT3L-Fc fusion proteins described herein additionally comprise the M428L and N434S mutations, which are collectively referred to as LS. In certain embodiments, the FLT3L-Fc fusion proteins described herein comprise the L234F, L235E, D265A, F405L, M428L, and N434S mutations, which are collectively referred to as “FEALLS.” In certain embodiments, the FLT3L-Fc fusion proteins described herein comprise the L234F, L235E, D265A, M428L, and N434S mutations along with one further mutation selected from the group consisting of F405L, F405A, F405D, F405E, F405H, F4051, F405K, F405M, F405N, F405Q, F4055, F405T, F405V, F405W, and F405Y. In certain embodiments, the FLT3L-Fc fusion proteins described herein comprise the L234F, L235E, D265A, K409R, M428L, and N434S mutations which are collectively referred to as “FEARLS.” In certain embodiments, FEALLS and FEARLS are comprised in a fusion protein described herein. By reducing or abrogating effector function on the Fc domains of the FLT3L-Fc fusion protein, cells bound by the molecule are not killed by innate effector cells e.g., NK cells, macrophages.

In certain embodiments, the one or more modifications are selected from the following Fc amino acid substitutions (EU numbering) or combinations thereof: L234F; L235E; G236A; S239D; F243L; D265E; D265A; S267E; H268F; R292P; N297Q; N297G, N297A; S298A; S324T; I332E; S239D; A330L; L234F; L235E; P331S; F243L; Y300L; V305I; P396L; S298A; E333A; K334A; E345R; L235V; F243L; R292P; Y300L; P396L; M428L; E430G; N434S; G236A, S267E, H268F, S324T, and I332E; G236A, S239D, and I332E; S239D, A330L, I332E; L234F, L235E, and P3315; F243L, R292P, Y300L, V305I, and P396L; G236A, H268F, S324T, and I332E; S239D, H268F, S324T, and I332E; S298A, E333A, and K334A; L235V, F243L, R292P, Y300L, and P396L; S239D, I332E; S239D, S298A, and I332E; G236A, S239D, I332E, M428L, and N434S; G236A, S239D, A330L, I332E, M428L, and N434S; S239D, I332E, G236A and A330L; M428L and N4343S; M428L, N434S; G236A, S239D, A330L, and I332E; and G236A and I332E. In certain embodiments, the one or more modifications is selected from the group consisting of: D265A, L234F, L235E, N297A, N297G, N297Q, and P331S. In certain embodiments, the one or more modifications are selected from N297A and D265A. In certain embodiments, the one or more modifications are selected from L234F and L235E. In certain embodiments, the one or more modifications are selected from L234F, L234E, and D265A. In certain embodiments, the one or more modifications are selected from L234F, L234E, and N297Q. In certain embodiments, the one or more modifications are selected from L234F, L235E, and P331S. In certain embodiments, the one or more modifications are selected from D265A and N297Q. In certain embodiments, the one or more modifications are selected from L234F, L235E, D265A, N297A, N297G, N297Q, and P331S.

Mutations that reduce Fc-receptor binding and find use in the herein described fusion proteins include, for example, N297A; N297G; N297Q; D265A; L234F/L235E; L234F/L235E/N297Q; L234F/L235E/P331S; D265A/N297Q; and L234F/L235E/D265A/N297Q/P331S (all EU numbering). In certain embodiments the FLT3L-Fc fusion proteins described herein described herein comprise L234F and L235E mutations. In certain embodiments the FLT3L-Fc fusion proteins described herein described herein comprise L234F, L235E, and D265A mutations. In certain embodiments the FLT3L-Fc fusion proteins described herein described herein comprise L234F, L235E, and N297Q mutations. In certain embodiments the FLT3L-Fc fusion proteins described herein described herein comprise an N297A or N297Q mutation. In certain embodiments the FLT3L-Fc fusion proteins described herein described herein comprise an N297A, N297G or N297Q mutation as well as L234F, L235E, and D265A mutations. In certain embodiments, one, two, three, four, or more amino acid substitutions are introduced into a Fc region to alter the effector function of the antigen binding molecule. For example, these substitutions are located at positions selected from the group consisting of amino acid residues 234, 235, 236, 237, 265, 297, 318, 320, and 322, (according to EU numbering). These positions can be replaced with a different amino acid residue such that the antigen binding molecule has an altered (e.g., reduced) affinity for an effector ligand (e.g., an Fc receptor or the C1 component of complement), but retains the antigen binding ability of the parent antibody. In certain embodiments, the FLT3L-Fc fusion proteins described herein described herein comprise E233P, L234V, L235A, and/or G236A mutations (EU numbering). In some embodiments, the FLT3L-Fc fusion proteins described herein comprise A327G, A330S, and/or P331S mutations (EU numbering). In some embodiments, the FLT3L-Fc fusion proteins described herein comprise K322A mutations (EU numbering). In some embodiments the FLT3L-Fc fusion proteins described herein comprise E318A, K320A, and K322A (EU numbering) mutations. In certain embodiments, the FLT3L-Fc fusion proteins described herein comprise a L235E (EU numbering) mutation.

In some embodiments, the Fc portion of the fusion protein comprises an amino acid sequence having at least 80%, at least 85%, at least 90%, at least 91%, at least 92%, at least 93%, at least 94%, at least 95%, at least 96%, at least 97%, at least 98%, at least 99%, or at least 100% identical to an amino acid sequence of

(SEQ ID NO: 103)
GGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQK
SLSLSPGK;
(SEQ ID NO: 104)
GGPSVFLFPPKPKDTLYITREPEVTCVVVDVSHEDPEVKFNWYVDGVEVHN
AKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQK
SLSLSPGK;
(SEQ ID NO: 105)
ESKYGPPCPPCPAPEFEGGPSVFLFPPKPKDTLMISRTPEVTCVVVDVSQE
DPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGNV
FSCSVMHEALHNHYTQKSLSLSLGK;
(SEQ ID NO: 106)
ESKYGPPCPPCPAPEAAGGPSVFLFPPKPKDTLMISRTPEVTCVVVDVSQE
DPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGNV
FSCSVMHEALHNHYTQKSLSLSLGK;
or
(SEQ ID NO: 107)
ESKYGPPCPPCPAPEFEGGPSVFLFPPKPKDTLYITREPEVTCVVVDVSQE
DPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGNV
FSCSVMHEALHNHYTQKSLSLSLGK.

Illustrative polypeptide sequences of the FLT3L-Fc fusion proteins described herein are provided in Table A. In some embodiments, FLT3-Fc fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27. In some embodiments, FLT3-Fc fusion protein comprises an amino acid sequence selected from the group consisting of SEQ ID NOs: 1-18 and 21-27. In some embodiments, FLT3-Fc fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 19-20. In some embodiments, FLT3-Fc fusion protein comprises an amino acid sequence selected from the group consisting of SEQ ID NOs: 19-20.

In various embodiments, the FLT3L-Fc fusion proteins may be glycosylated or aglycosylated. In certain embodiments where the FLT3L-Fc fusion protein is glycosylated, at least 50%, at least 60%, at least 70%, least 80%, at least 85%, at least 90%, or more, N-linked and/or O-linked glycosylation sites in the fusion protein are sialylated. In certain embodiments where the FLT3L-Fc fusion protein is sialylated, the sialylated N-linked and/or O-linked glycosylation sites in the fusion protein comprise from 2 to 7 sialic acid residues, e.g., from 3 to 6 sialic acid residues, e.g., from 4 to 5 sialic acid residues.

In some embodiments, the FLT3L-Fc fusion proteins have a serum half-life of at least about 7 days, e.g., in a mammal, e.g., in a human, monkey, mouse, cat or dog. In some embodiments, the FLT3L-Fc fusion proteins have a serum half-life of at least about 7 days, e.g., at least about 8, 9, 10, 12, 14, 16, 18, 20, 22, 24, 26, 28, 30 days, or longer, e.g., in a mammal, e.g., in a human, monkey, mouse, cat or dog. Generally, a shorter serum half-life is observed with relatively lower doses. A longer serum half-life is observed with relatively higher doses.

Functionally, the FLT3L-Fc fusion proteins described herein induce, promote and/or increase the growth, proliferation and/or expansion of cells or populations of cells that express or overexpress FLT3 on their cell surface. Illustrative cells or populations of cells that express or overexpress FLT3 include dendritic cells (e.g., cDC1 cells and/or cDC2 cells), monocyte-derived dendritic cells (moDCs), and/or progenitor cells thereof. In some embodiments, the cell or population of cells that express FLT3 comprise hematopoietic progenitor cells, e.g., Common Lymphoid Progenitors (CLPs), Early Progenitors with Lymphoid and Myeloid potential (EPLMs), granulocyte-monocyte (GM) progenitors (GMP), monocyte-derived dendritic cells (moDCs) progenitors, and early multi-potent progenitors (MPP) within the Lineage-kit+Sca1 (LSK) compartment.

TABLE A
FLT3L-Fc fusion proteins

| PROTEIN NO: | | |
|---|---|---|
| SEQ ID NO: | Features | Polypeptide Sequence (Fc domain is underlined) |
| 1 | FLT3L ECD-hingeless hG1 | TQDCSFQHSPISSDFAVKIRELSDYLLQDYPVTVASNLQDEELCGGLWRLVLAQRWMER |
| | | LKTVAGSKMQGLLERVNTEIHFVTKCAFQPPPSCLRFVQTNISRLLQETSEQLVALKPWIT |
| | | RQNFSRCLELQCQPDSSTLPPPWSPRPLEATAPTAPQGGPSVFLFPPKPKDTLMISRTPEVT |
| | | CVVVDVSHEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCSVMHEALHNHY |
| | | TQKSLSLSPGK |
| 2 | FLT3L ECD (Δ C-term 5aa | TQDCSFQHSPISSDFAVKIRELSDYLLQDYPVTVASNLQDEELCGGLWRLVLAQRWMER |
| | (PTAPQ; SEQ ID NO: 85)) - | LKTVAGSKMQGLLERVNTEIHFVTKCAFQPPPSCLRFVQTNISRLLQETSEQLVALKPWIT |
| | hingeless hG1 | RQNFSRCLELQCQPDSSTLPPPWSPRPLEATAGGPSVFLFPPKPKDTLMISRTPEVTCVVV |
| | | DVSHEDPEVKFNWYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMHEALHNHYTQK |
| | | SLSLSPGK |
| 3 | FLT3L ECD - hG4 | TQDCSFQHSPISSDFAVKIRELSDYLLQDYPVTVASNLQDEELCGGLWRLVLAQRWMER |
| | S228P/L235E | LKTVAGSKMQGLLERVNTEIHFVTKCAFQPPPSCLRFVQTNISRLLQETSEQLVALKPWIT |
| | | RQNFSRCLELQCQPDSSTLPPPWSPRPLEATAPTAPQESKYGPPCPPCPAPEFEGGPSVFLF |
| | | PPKPKDTLMISRTPEVTCVVVDVSQEDPEVQFNWYVDGVEVHNAKTKPREEQFNSTYRV |
| | | VSVLTVLHQDWLNGKEYKCKVSNKGLPSSIEKTISKAKGQPREPQVYTLPPSQEEMTKN |
| | | QVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| 4 | FLT3L ECD - hG4 | TQDCSFQHSPISSDFAVKIRELSDYLLQDYPVTVASNLQDEELCGGLWRLVLAQRWMER |
| | S228P/F234A/L235A | LKTVAGSKMQGLLERVNTEIHFVTKCAFQPPPSCLRFVQTNISRLLQETSEQLVALKPWIT |
| | | RQNFSRCLELQCQPDSSTLPPPWSPRPLEATAPTAPQESKYGPPCPPCPAPEAAGGPSVFL |
| | | FPPKPKDTLMISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| 5 | Aglyco-FLT3L ECD | TQDCSFQHSPISSDFAVKIRELSDYLLQDYPVTVASNLQDEELCGGLWRLVLAQRWMER |
| | (S128A/S151A) hingeless | LKTVAGSKMQGLLERVNTEIHFVTKCAFQPPPSCLRFVQTNIARLLQETSEQLVALKPWI |
| | hG1 | TRQNFARCLELQCQPDSSTLPPPWSPRPLEATAPTAPQGGPSVFLFPPKPKDTLMISRTPEV |
| | | TCVVVDVSHEDPEVKFNWYVDGVEVHNAKTKPREEQYNSTYRVVSVLTVLHQDWLNG |
| | | KEYKCKVSNKALPAPIEKTISKAKGQPREPQVYTLPPSREEMTKNQVSLTCLVKGFYPSDI |
| | | AVEWESNGQPENNYKTTPPVLDSDGSFFLYSKLTVDKSRWQQGNVFSCSVMHEALHNH |
| | | YTQKSLSLSPGK |
| 6 | FLT3L (Δ C-term 5aa | TQDCSFQHSPISSDFAVKIRELSDYLLQDYPVTVASNLQDEELCGGLWRLVLAQRWMER |
| | (PTAPQ; SEQ ID NO: 85)) - | LKTVAGSKMQGLLERVNTEIHFVTKCAFQPPPSCLRFVQTNISRLLQETSEQLVALKPWIT |
| | hG4 S228P/F234A/L235A | RQNFSRCLELQCQPDSSTLPPPWSPRPLEATAESKYGPPCPPCPAPEAAGGPSVFLFPPKPK |
| | | DTLMISRTPEVTCVVVDVSQEDPEVQFNWYVDGVEVHNAKTKPREEQFNSTYRVVSVL |
| | | TVLHQDWLNGKEYKCKVSNKGLPSSIEKTISKAKGQPREPQVYTLPPSQEEMTKNQVSL |
| | | TCLVKGFYPSDIAVEWESNGQPENNYKTTPPVLDSDGSFFLYSRLTVDKSRWQEGNVFS |
| | | CSVMHEALHNHYTQKSLSLSLGK |
| 7 | FLT3L ECD (Δ C-term 10aa | TQDCSFQHSPISSDFAVKIRELSDYLLQDYPVTVASNLQDEELCGGLWRLVLAQRWMER |
| | (LEATAPTAPQ; SEQ ID | LKTVAGSKMQGLLERVNTEIHFVTKCAFQPPPSCLRFVQTNISRLLQETSEQLVALKPWIT |
| | NO: 90)) -hingeless hG1 | RQNFSRCLELQCQPDSSTLPPPWSPRPGGPSVFLFPPKPKDTLMISRTPEVTCVVVDVSHE |
| | | DPEVKFNWYVDGVEVHNAKTKPREEQYNSTYRVVSVLTVLHQDWLNGKEYKCKVSNK |
| | | ALPAPIEKTISKAKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNHYTQKSLSLSPG |
| | | K |
| 8 | FLT3L ECD (Δ C-term 10aa | TQDCSFQHSPISSDFAVKIRELSDYLLQDYPVTVASNLQDEELCGGLWRLVLAQRWMER |
| | (LEATAPTAPQ; SEQ ID | LKTVAGSKMQGLLERVNTEIHFVTKCAFQPPPSCLRFVQTNISRLLQETSEQLVALKPWIT |
| | NO: 90))-hG4 | RQNFSRCLELQCQPDSSTLPPPWSPRPESKYGPPCPPCPAPEAAGGPSVFLFPPKPKDTLMI |
| | S228P/F234A/L235A | SRTPEVTCVVVDVSQEDPEVQFNWYVDGVEVHNAKTKPREEQFNSTYRVVSVLTVLHQ |
| | | DWLNGKEYKCKVSNKGLPSSIEKTISKAKGQPREPQVYTLPPSQEEMTKNQVSLTCLVK |
| | | GFYPSDIAVEWESNGQPENNYKTTPPVLDSDGSFFLYSRLTVDKSRWQEGNVFSCSVMH |
| | | EALHNHYTQKSLSLSLGK |
| 9 | FLT3L ECD-hingeless hG1 | TQDCSFQHSPISSDFAVKIRELSDYLLQDYPVTVASNLQDEELCGGLWRLVLAQRWMER |
| | (M252Y/S254T/T256E) | LKTVAGSKMQGLLERVNTEIHFVTKCAFQPPPSCLRFVQTNISRLLQETSEQLVALKPWIT |
| | | RQNFSRCLELQCQPDSSTLPPPWSPRPLEATAPTAPQGGPSVFLFPPKPKDTLYITREPEVT |
| | | CVVVDVSHEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCSVMHEALHNHY |
| | | TQKSLSLSPGK |
| 10 | FLT3L ECD (Δ C-term 5aa | TQDCSFQHSPISSDFAVKIRELSDYLLQDYPVTVASNLQDEELCGGLWRLVLAQRWMER |
| | (PTAPQ; SEQ ID NO: 85)) - | LKTVAGSKMQGLLERVNTEIHFVTKCAFQPPPSCLRFVQTNISRLLQETSEQLVALKPWIT |
| | hingeless hG1 | RQNFSRCLELQCQPDSSTLPPPWSPRPLEATAGGPSVFLFPPKPKDTLYITREPEVTCVVV |
| | (M252Y/S254T/T256E) | DVSHEDPEVKFNWYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMHEALHNHYTQK |
| | | SLSLSPGK |
| 11 | FLT3L ECD - hG4 | TQDCSFQHSPISSDFAVKIRELSDYLLQDYPVTVASNLQDEELCGGLWRLVLAQRWMER |
| | S228P/L235E/ | LKTVAGSKMQGLLERVNTEIHFVTKCAFQPPPSCLRFVQTNISRLLQETSEQLVALKPWIT |
| | M252Y/S254T/T256E) | RQNFSRCLELQCQPDSSTLPPPWSPRPLEATAPTAPQESKYGPPCPPCPAPEFEGGPSVFLF |
| | | PPKPKDTLYITREPEVTCVVVDVSQEDPEVQFNWYVDGVEVHNAKTKPREEQFNSTYRV |
| | | VSVLTVLHQDWLNGKEYKCKVSNKGLPSSIEKTISKAKGQPREPQVYTLPPSQEEMTKN |
| | | QVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| 12 | FLT3L ECD - hG4 | TQDCSFQHSPISSDFAVKIRELSDYLLQDYPVTVASNLQDEELCGGLWRLVLAQRWMER |
| | (S228P/F234A/L235A/ | LKTVAGSKMQGLLERVNTEIHFVTKCAFQPPPSCLRFVQTNISRLLQETSEQLVALKPWIT |
| | M252Y/S254T/T256E) | RQNFSRCLELQCQPDSSTLPPPWSPRPLEATAPTAPQESKYGPPCPPCPAPEAAGGPSVFL |
| | | FPPKPKDTLYITREPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQE |
| | | GNVFSCSVMHEALHNHYTQKSLSLSLGK |
| 13 | Aglyco-FLT3L ECD | TQDCSFQHSPISSDFAVKIRELSDYLLQDYPVTVASNLQDEELCGGLWRLVLAQRWMER |
| | (S128A/S151A) hingeless | LKTVAGSKMQGLLERVNTEIHFVTKCAFQPPPSCLRFVQTNIARLLQETSEQLVALKPWI |
| | hG1 | TRQNFARCLELQCQPDSSTLPPPWSPRPLEATAPTAPQGGPSVFLFPPKPKDTLYITREPEV |
| | (M252Y/S254T/T256E) | TCVVVDVSHEDPEVKFNWYVDGVEVHNAKTKPREEQYNSTYRVVSVLTVLHQDWLNG |
| | | KEYKCKVSNKALPAPIEKTISKAKGQPREPQVYTLPPSREEMTKNQVSLTCLVKGFYPSDI |
| | | AVEWESNGQPENNYKTTPPVLDSDGSFFLYSKLTVDKSRWQQGNVFSCSVMHEALHNH |
| | | YTQKSLSLSPGK |
| 14 | FLT3L ECD (Δ C-term 5aa | TQDCSFQHSPISSDFAVKIRELSDYLLQDYPVTVASNLQDEELCGGLWRLVLAQRWMER |
| | (PTAPQ; SEQ ID NO: 85)) - | LKTVAGSKMQGLLERVNTEIHFVTKCAFQPPPSCLRFVQTNISRLLQETSEQLVALKPWIT |
| | hG4 | RQNFSRCLELQCQPDSSTLPPPWSPRPLEATAESKYGPPCPPCPAPEAAGGPSVFLFPPKPK |
| | (S228P/F234A/L235A/ | DTLYITREPEVTCVVVDVSQEDPEVQFNWYVDGVEVHNAKTKPREEQFNSTYRVVSVL |
| | M252Y/S254T/T256E) | TVLHQDWLNGKEYKCKVSNKGLPSSIEKTISKAKGQPREPQVYTLPPSQEEMTKNQVSL |
| | | TCLVKGFYPSDIAVEWESNGQPENNYKTTPPVLDSDGSFFLYSRLTVDKSRWQEGNVFS |
| | | CSVMHEALHNHYTQKSLSLSLGK |
| 15 | FLT3L ECD (Δ C-term 10aa | TQDCSFQHSPISSDFAVKIRELSDYLLQDYPVTVASNLQDEELCGGLWRLVLAQRWMER |
| | (LEATAPTAPQ; SEQ ID | LKTVAGSKMQGLLERVNTEIHFVTKCAFQPPPSCLRFVQTNISRLLQETSEQLVALKPWIT |
| | NO: 90))-hG1 | RQNFSRCLELQCQPDSSTLPPPWSPRPGGPSVFLFPPKPKDTLYITREPEVTCVVVDVSHE |
| | (M252Y/S254T/T256E) | DPEVKFNWYVDGVEVHNAKTKPREEQYNSTYRVVSVLTVLHQDWLNGKEYKCKVSNK |
| | | ALPAPIEKTISKAKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNHYTQKSLSLSPG |
| | | K |
| 16 | FLT3L ECD (Δ C-term 10aa | TQDCSFQHSPISSDFAVKIRELSDYLLQDYPVTVASNLQDEELCGGLWRLVLAQRWMER |
| | (LEATAPTAPQ; SEQ ID | LKTVAGSKMQGLLERVNTEIHFVTKCAFQPPPSCLRFVQTNISRLLQETSEQLVALKPWIT |
| | NO: 90))-hG4 | RQNFSRCLELQCQPDSSTLPPPWSPRPESKYGPPCPPCPAPEAAGGPSVFLFPPKPKDTLYI |
| | (S228P/F234A/L235A/ | TREPEVTCVVVDVSQEDPEVQFNWYVDGVEVHNAKTKPREEQFNSTYRVVSVLTVLHQ |
| | M252Y/S254T/T256E) | DWLNGKEYKCKVSNKGLPSSIEKTISKAKGQPREPQVYTLPPSQEEMTKNQVSLTCLVK |
| | | GFYPSDIAVEWESNGQPENNYKTTPPVLDSDGSFFLYSRLTVDKSRWQEGNVFSCSVMH |
| | | EALHNHYTQKSLSLSLGK |
| 17 | Aglyco-FLT3L ECD (Δ | TQDCSFQHSPISSDFAVKIRELSDYLLQDYPVTVASNLQDEELCGGLWRLVLAQRWMER |
| | C-term 10aa (LEATAPTAPQ; | LKTVAGSKMQGLLERVNTEIHFVTKCAFQPPPSCLRFVQTNIARLLQETSEQLVALKPWI |
| | SEQ ID NO: 90)) | TRQNFARCLELQCQPDSSTLPPPWSPRPESKYGPPCPPCPAPEAAGGPSVFLFPPKPKDTL |
| | (S128A/S151A) - hG4 | MISRTPEVTCVVVDVSQEDPEVQFNWYVDGVEVHNAKTKPREEQFNSTYRVVSVLTVL |
| | S228P/F234A/L235A | HQDWLNGKEYKCKVSNKGLPSSIEKTISKAKGQPREPQVYTLPPSQEEMTKNQVSLTCL |
| | | VKGFYPSDIAVEWESNGQPENNYKTTPPVLDSDGSFFLYSRLTVDKSRWQEGNVFSCSV |
| | | MHEALHNHYTQKSLSLSLGK |
| 18 | Aglyco-FLT3L ECD (Δ | TQDCSFQHSPISSDFAVKIRELSDYLLQDYPVTVASNLQDEELCGGLWRLVLAQRWMER |
| | C-term 10aa (LEATAPTAPQ; | LKTVAGSKMQGLLERVNTEIHFVTKCAFQPPPSCLRFVQTNIARLLQETSEQLVALKPWI |
| | SEQ ID NO: 90)) | TRQNFARCLELQCQPDSSTLPPPWSPRPESKYGPPCPPCPAPEAAGGPSVFLFPPKPKDTL |
| | (S128A/S151A) - hG4 | YITREPEVTCVVVDVSQEDPEVQFNWYVDGVEVHNAKTKPREEQFNSTYRVVSVLTVL |
| | (S228P/F234A/L235A/ | HQDWLNGKEYKCKVSNKGLPSSIEKTISKAKGQPREPQVYTLPPSQEEMTKNQVSLTCL |
| | M252Y/S254T/T256E) | VKGFYPSDIAVEWESNGQPENNYKTTPPVLDSDGSFFLYSRLTVDKSRWQEGNVFSCSV |
| | | MHEALHNHYTQKSLSLSLGK |
| 19 | Murine surrogate | TPDCYFSHSPISSNFKVKFRELTDHLLKDYPVTVAVNLQDEKHCKALWSLFLAQRWIEQL |
| | mFLT3L ECD - mG2a Fc | KTVAGSKMQTLLEDVNTEIHFVTSCTFQPLPECLRFVQTNISHLLKDTCTQLLALKPCIGK |
| | (L234A/L235A/P329G) | ACQNFSRCLEVQCQPDSSTLLPPRSPIALEATELPEPRGPTIKPCPPCKCPAPNAAGGPSVFI |
| | | FPPKIKDVLMISLSPIVTCVVVDVSEDDPDVQISWFVNNVEVHTAQTQTHREDYNSTLRV |
| | | VSALPIQHQDWMSGKEFKCKVNNKDLGAPIERTISKPKGSVRAPQVYVLPPPEEEMTKK |
| | | QVTLTCMVTDFMPEDIYVEWTNNGKTELNYKNTEPVLDSDGSYFMYSKLRVEKKNWV |
| | | ERNSYSCSVVHEGLHNHHTTKSFSRTPGK |
| 20 | Murine surrogate | TPDCYFSHSPISSNFKVKFRELTDHLLKDYPVTVAVNLQDEKHCKALWSLFLAQRWIEQL |
| | mFLT3L ECD (C136S) | KTVAGSKMQTLLEDVNTEIHFVTSCTFQPLPECLRFVQTNISHLLKDTSTQLLALKPCIGK |
| | mG2aFc | ACQNFSRCLEVQCQPDSSTLLPPRSPIALEATELPEPRGPTIKPCPPCKCPAPNAAGGPSVFI |
| | (L234A/L235A/P329G) | FPPKIKDVLMISLSPIVTCVVVDVSEDDPDVQISWFVNNVEVHTAQTQTHREDYNSTLRV |
| | | VSALPIQHQDWMSGKEFKCKVNNKDLGAPIERTISKPKGSVRAPQVYVLPPPEEEMTKK |
| | | QVTLTCMVTDFMPEDIYVEWTNNGKTELNYKNTEPVLDSDGSYFMYSKLRVEKKNWV |
| | | ERNSYSCSVVHEGLHNHHTTKSFSRTPGK |
| 21 | FLT3L ECD-hingeless | TQDCSFQHSPISSDFAVKIRELSDYLLQDYPVTVASNLQDEELCGGLWRLVLAQRWMER |
| | monoFc | LKTVAGSKMQGLLERVNTEIHFVTKCAFQPPPSCLRFVQTNISRLLQETSEQLVALKPWIT |
| | | RQNFSRCLELQCQPDSSTLPPPWSPRPLEATAPTAPQGGPSVFLFPPKPKDTLMISRTPEVT |
| | | CVVVDVSHEDPEVKFNWYVDGVEVHNAKTKPREEQYNSTYRVVSVLTVLHQDWLNGK |
| | | EYKCKVSNKALPAPIEKTISKAKGQPREPQVYTKPPSREEMTKNQVSLSCLVKGFYPSDIA |
| | | VEWESNGQPENNYKTTVPVLDSDGSFRLASYLTVDKSRWQQGNVFSCSVMHEALHNHY |
| | | TQKSLSLSPGK |
| 22 | FLT3L ECD (H8Y) - | TQDCSFQYSPISSDFAVKIRELSDYLLQDYPVTVASNLQDEELCGGLWRLVLAQRWMER |
| | hingeless hG1 Fc | LKTVAGSKMQGLLERVNTEIHFVTKCAFQPPPSCLRFVQTNISRLLQETSEQLVALKPWIT |
| | | RQNFSRCLELQCQPDSSTLPPPWSPRPLEATAPTAPQGGPSVFLFPPKPKDTLMISRTPEVT |
| | | CVVVDVSHEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCSVMHEALHNHY |
| | | TQKSLSLSPGK |
| 23 | FLT3L ECD (K84E) - | TQDCSFQHSPISSDFAVKIRELSDYLLQDYPVTVASNLQDEELCGGLWRLVLAQRWMER |
| | hingeless hG1 Fc | LKTVAGSKMQGLLERVNTEIHFVTECAFQPPPSCLRFVQTNISRLLQETSEQLVALKPWIT |
| | | RQNFSRCLELQCQPDSSTLPPPWSPRPLEATAPTAPQGGPSVFLFPPKPKDTLMISRTPEVT |
| | | CVVVDVSHEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCSVMHEALHNHY |
| | | TQKSLSLSPGK |
| 24 | FLT3L ECD (H8Y/K84E) | TQDCSFQYSPISSDFAVKIRELSDYLLQDYPVTVASNLQDEELCGGLWRLVLAQRWMER |
| | hingeless hG1 Fc | LKTVAGSKMQGLLERVNTEIHFVTECAFQPPPSCLRFVQTNISRLLQETSEQLVALKPWIT |
| | | RQNFSRCLELQCQPDSSTLPPPWSPRPLEATAPTAPQGGPSVFLFPPKPKDTLMISRTPEVT |
| | | CVVVDVSHEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCSVMHEALHNHY |
| | | TQKSLSLSPGK |
| 25 | Aglyco-FLT3L ECD | TQDCSFQHSPISSDFAVKIRELSDYLLQDYPVTVASNLQDEELCGGLWRLVLAQRWMER |
| | (Δ C-term 5aa (PTAPQ; | LKTVAGSKMQGLLERVNTEIHFVTKCAFQPPPSCLRFVQTNIARLLQETSEQLVALKPWI |
| | SEQ ID NO: 85)) | TRQNFARCLELQCQPDSSTLPPPWSPRPLEATAESKYGPPCPPCPAPEAAGGPSVFLFPPK |
| | (S128A/S151A) - hG4 | PKDTLYITREPEVTCVVVDVSQEDPEVQFNWYVDGVEVHNAKTKPREEQFNSTYRVVS |
| | (S228P/F234A/L235A/ | VLTVLHQDWLNGKEYKCKVSNKGLPSSIEKTISKAKGQPREPQVYTLPPSQEEMTKNQV |
| | M252Y/S254T/T256E) | SLTCLVKGFYPSDIAVEWESNGQPENNYKTTPPVLDSDGSFFLYSRLTVDKSRWQEGNVF |
| | | SCSVMHEALHNHYTQKSLSLSLGK |
| 26 | FLT3L ECD (Δ C-term 5aa | TQDCSFQHSPISSDFAVKIRELSDYLLQDYPVTVASNLQDEELCGGLWRLVLAQRWMER |
| | (PTAPQ; SEQ ID NO: 85)) - | LKTVAGSKMQGLLERVNTEIHFVTKCAFQPPPSCLRFVQTNISRLLQETSEQLVALKPWIT |
| | linker SST/AAA - hG4 | RQNFSRCLELQCQPDAAALPPPWSPRPLEATAESKYGPPCPPCPAPEAAGGPSVFLFPPKP |
| | (S228P/F234A/L235A/ | KDTLYITREPEVTCVVVDVSQEDPEVQFNWYVDGVEVHNAKTKPREEQFNSTYRVVSV |
| | M252Y/S254T/T256E) | LTVLHQDWLNGKEYKCKVSNKGLPSSIEKTISKAKGQPREPQVYTLPPSQEEMTKNQVS |
| | | LTCLVKGFYPSDIAVEWESNGQPENNYKTTPPVLDSDGSFFLYSRLTVDKSRWQEGNVFS |
| | | CSVMHEALHNHYTQKSLSLSLGK |
| 27 | FLT3L ECD (Δ C-term 5aa | TQDCSFQHSPISSDFAVKIRELSDYLLQDYPVTVASNLQDEELCGGLWRLVLAQRWMER |
| | (PTAPQ; SEQ ID NO: 85)) - | LKTVAGSKMQGLLERVNTEIHFVTKCAFQPPPSCLRFVQTNISRLLQETSEQLVALKPWIT |
| | linker SST/AAA; | RQNFSRCLELQCQPDAAALPPPWAPRPLEATAEAKYGPPCPPCPAPEAAGGPSVFLFPPK |
| | S170A/S180A - hG4 | PKDTLYITREPEVTCVVVDVSQEDPEVQFNWYVDGVEVHNAKTKPREEQFNSTYRVVS |
| | (S228P/F234A/L235A/ | VLTVLHQDWLNGKEYKCKVSNKGLPSSIEKTISKAKGQPREPQVYTLPPSQEEMTKNQV |
| | M252Y/S254T/T256E) | SLTCLVKGFYPSDIAVEWESNGQPENNYKTTPPVLDSDGSFFLYSRLTVDKSRWQEGNVF |
| | | SCSVMHEALHNHYTQKSLSLSLGK |

### Heterodimers and Fusion Proteins Comprising a FLT3L-Fc Fusion Protein and a Second Polypeptide

Further provided are fusion proteins comprising (i) a FLT3L-Fc fusion protein described herein, e.g., having an amino acid sequence that is at least 80%, at least 85%, at least 90%, at least 91%, at least 92%, at least 93%, at least 94%, at least 95%, at least 96%, at least 97%, at least 98%, at least 99%, or 100%, identical to an amino acid sequence selected from the group consisting of SEQ ID NOs: 19-20, and (ii) a second polypeptide. In some embodiments, the second polypeptide comprises a targeting moiety or domain, a growth factor, a cytokine, a chemokine or a TNF superfamily (TNFSF) member. In some embodiments, the second polypeptide is N-terminal to the FLT3L extracellular domain. In some embodiments, the second polypeptide is C-terminal to the Fc region. In some embodiments, the second polypeptide is between the FLT3L extracellular domain and the Fc region. In various embodiments, the targeting moiety binds to a protein target in Table B.

Further provided are heterodimeric molecules comprising (i) a FLT3L-Fc fusion protein described herein, e.g., having an amino acid sequence that is at least 80%, at least 85%, at least 90%, at least 91%, at least 92%, at least 93%, at least 94%, at least 95%, at least 96%, at least 97%, at least 98%, at least 99%, or 100%, identical to an amino acid sequence selected from the group consisting of SEQ ID NOs: 19-20, and (ii) a second polypeptide fused to a second Fc region. In certain embodiments, the first and second Fc regions of the heterodimeric molecules are different, e.g., having complementary “knob (W)-and-hole (S)” amino acid substitutions at position 366 (EU numbering). In some embodiments, the second polypeptide comprises a targeting moiety or domain, a growth factor, a cytokine, a chemokine or a TNF superfamily (TNFSF) member. In various embodiments, the targeting moiety binds to a protein target in Table B.

In some embodiments, the targeting moiety or domain comprises an antibody fragment (e.g., scFv, sc(Fv)₂, Fab, F(ab)₂, Fab′, F(ab′)₂, Facb, and Fv). In some embodiments, the antibody fragment comprises a Fab or a single-chain variable fragment (scFv). In some embodiments, both the first Fc region and the second Fc region do not comprise a hinge region. In some embodiments, the heterodimer is stabilized by an interaction between the first Fc region and the second Fc region. Illustrative interactions that can stabilize the heterodimer through the Fc region include without limitation disulfide bonds and complementary amino acid substitutions in the first and second Fc regions (e.g., knob-in-hole mutations).

In some embodiments, the targeting moiety or domain comprises a non-immunoglobulin or antibody mimetic protein. Examples of non-immunoglobulin or antibody mimetic protein targeting moieties or domains include without limitation adnectins, affibody molecules, affilins, affimers, affitins, alphabodies, anticalins, peptide aptamers, armadillo repeat proteins (ARMs), atrimers, avimers, designed ankyrin repeat proteins (DARPins®), fynomers, knottins, Kunitz domain peptides, monobodies, and nanoCLAMPs. Non-immunoglobulin or antibody mimetic protein targeting moieties or domains of use in the herein described FLT3L-Fc fusion protein heterodimers are described, e.g., in Zhang, et al., Methods Mol Biol. 2017; 1575:3-13; Ta, et al., Future Med Chem. 2017 August; 9(12):1301-1304; Yu, et al., Annu Rev Anal Chem (Palo Alto Calif.). 2017 Jun. 12; 10(1):293-320; Baloch, et al., Crit Rev Biotechnol. 2016; 36(2):268-75; and Bruce, et al., Chembiochem. 2016 Oct. 17; 17(20):1892-1899.

In some embodiments, the targeting moiety or domain has T-cell receptor (TCR)-like binding properties and binds to the epitope of a target or tumor-associated antigen (TAA) presented in a major histocompatibility complex (MHC) molecule.

In some embodiments, the targeting moiety or domain comprises a binding partner domain, e.g., a soluble or extracellular domain of the binding partner or ligand of the protein target or antigen. For example, in some embodiments, the targeting moiety or domain comprises a binding partner or ligand of any of the protein or antigen targets listed in Table B. In one embodiment, the targeting moiety or domain comprises the extracellular domain of a TGFB1 receptor (e.g., a “TGF beta trap”).

In homodimers or heterodimer formats of the FLT3L-Fc fusion proteins, the dimeric molecule comprises first and second Fc domains. In certain embodiments, amino acid substitutions may be in one or both of the first and second Fc domains. In certain embodiments, the one or both of the first and second Fc domains have one or more (1, 2, 3, 4, or 5) of the following mutations (EU numbering). In some embodiments, Fc region heterodimerization of the two different immunoadhesins (Fc fusion proteins) can be facilitated by so-called ‘knobs-into-holes’ mutations (Atwell et al. 1997. JMB 270:26-35). The ‘hole’ mutations (T366S, L368A and Y407V) are incorporated into one Fc-containing chain, the T366W ‘knob’ mutation is incorporated into the other chain. Knob-and-hole amino acid substitutions can be incorporated into human IgG1 or human IgG4 Fc domains. In addition, a C220S mutation can be incorporated into an IgG1 hinge region of a scFv-containing arm to eliminate a free cysteine that otherwise forms a disulfide bond with a corresponding cysteine in the light chain in a wild-type IgG1. Co-transfection of such constructs leads to preferential formation of a heterodimeric Fc, with low levels of homodimer contaminants. Additionally, incorporating a S354C mutation can be incorporated into the Fc containing the ‘knob’ mutations and a Y349C mutation into the Fc containing the ‘hole’ mutations can optionally be used to generate a covalent bond between the two halves of the heterodimeric Fc if additional thermodynamic stability is desired (Merchant et al. 1998. Nat. Biotechnol. 16: 677-81). In certain embodiments, R409D, K370E mutations are introduced in the “knobs chain” and D399K, E357K mutations in the “hole chain.” In other embodiments, Y349C, T366W mutations are introduced in one of the chains and E356C, T366S, L368A, Y407V mutations in the counterpart chain. In some embodiments. Y349C, T366W mutations are introduced in one chain and S354C, T366S, L368A, Y407V mutations in the counterpart chain. In some embodiments, Y349C, T366W mutations are introduced in one chain and S354C, T366S, L368A, Y407V mutations in the counterpart chain. In yet other embodiments, Y349C, T366W mutations are introduced in one chain and S354C, T366S, L368A, Y407V mutations in the counterpart chain (all EU numbering).

To facilitate purification of the heterodimeric molecule away from contaminating homodimeric products, the H435R or H435R+Y436F mutations to reduce or eliminate protein A binding can be introduced into one but not both of the Fc-containing chains (Jendeberg, L. et al. 1997 J. Immunol. Methods 201:25-34). This reduces or eliminates protein A binding of the homodimer contaminant containing these mutations, and greatly simplifies purification of the desired heterodimer away from remaining homodimer contaminant via additional chromatography steps (e.g., ion exchange). In embodiments incorporating H435R (or H435R+Y436F) mutations in the first or second Fc region of a heavy chain, if the VH region in the same heavy chain is from a VH3 family variable region, this VH region can also include amino acid substitutions, as described herein, to reduce or eliminate Protein A binding of the entire heavy chain.

Yet another exemplary method of making bispecific antibodies is by using the Trifunctional Hybrid Antibodies platform—Triomab®. This platform employs a chimeric construction made up of half of two full-length antibodies of different isotypes, mouse IgG2a and rat IgG2b. This technology relies on species-preferential heavy/light chain pairing associations. See, Lindhofer et al., J Immunol., 155:219-225 (1995).

Yet another method for making bispecific antibodies is the CrossMab technology. CrossMab are chimeric antibodies constituted by the halves of two full-length antibodies. For correct chain pairing, it combines two technologies: (i) the knob-into-hole which favors a correct pairing between the two heavy chains; and (ii) an exchange between the heavy and light chains of one of the two Fabs to introduce an asymmetry which avoids light-chain mispairing. See, Ridgway et al., Protein Eng., 9:617-621 (1996); Schaefer et al., PNAS, 108:11187-11192 (2011). CrossMabs can combine two or more antigen binding domains for targeting two or more targets or for introducing bivalency towards one target such as the 2:1 format.

In some embodiments, the targeting moiety or domain targets or binds to an effector cell, e.g., engaging or activating a T-cell or an NK cell. In certain embodiments, the targeting moiety or domains binds to CD3. In some embodiments, the targeting moiety binds to CD16. Illustrative proteins and antigens, including tumor-associated antigens, immune checkpoint proteins and dendritic cell surface proteins, that can be targeted or bound by the targeting moiety or domain, include without limitation those listed in Table B. Target names, symbols (official and alternative) and Gene IDs identified in Table B are from ncbi.nlm.nih.gov/gene.

TABLE B
Illustrative Antigen/Protein Targets

| | NCBI Official | NCBI Human | Alternative Symbols |
|---|---|---|---|
| Target Name | Symbol | Gene ID No. | (also known as) |
| 5′-nucleotidase ecto | NT5E | 4907 | NT; eN; NT5; NTE; eNT; CD73; E5NT; CALJA |
| ALK receptor tyrosine kinase | ALK | 238 | CD246; NBLST3 |
| Alpha fetoprotein | AFP | 174 | AFPD, FETA, HPAFP |
| B and T lymphocyte associated | BTLA | 151888 | BTLA1, CD272 |
| cadherin 3 | CDH3 | 1001 | CDHP; HJMD; p-cadherin; PCAD |
| carbonic anhydrase 6 | CA6 | 765 | CA-VI; GUSTIN |
| carbonic anhydrase 9 | CA9 | 768 | MN; CAIX |
| carcinoembryonic antigen related cell | CEACAM3 | 1084 | CEA; CGM1; W264; W282; CD66D |
| adhesion molecule 3 | | | |
| carcinoembryonic antigen related cell | CEACAM5 | 1048 | CEA; CD66e |
| adhesion molecule 5 | | | |
| carcinoembryonic antigen related cell | CEACAM6 | 4680 | NCA; CEAL; CD66c |
| adhesion molecule 6 | | | |
| C—C motif chemokine receptor 2 | CCR2 | 729230 | CC-CKR-2, CCR-2, CCR2A, CCR2B, CD192, CKR2, |
| | | | CKR2A, CKR2B, CMKBR2, MCP-1-R |
| C—C motif chemokine receptor 4 | CCR4 | 1233 | CC-CKR-4, CD194, CKR4, CMKBR4, ChemR13, |
| | | | HGCN: 14099, K5-5 |
| C—C motif chemokine receptor 5 | CCR5 | 1234 | CC-CKR-5, CCCKR5, CCR-5, CD195, CKR-5, |
| | | | CKR5, CMKBR5, IDDM22 |
| C—C motif chemokine receptor 8 | CCR8 | 1237 | CC-CKR-8, CCR-8, CDw198, CKRL1, CMKBR8, |
| | | | CMKBRL2, CY6, GPRCY6, TER1 |
| CD160 molecule | CD160 | 11126 | BY55, NK1, NK28 |
| CD19 molecule | CD19 | 930 | B4; CVID3 |
| CD1a molecule | CD1A | 909 | CD1, FCB6, HTA1, R4, T6 |
| CD1c molecule | CD1C | 911 | R7; CD1; CD1A; BDCA1 |
| CD1d molecule | CD1D | 912 | CD1A, R3, R3G1 |
| CD1e molecule | CD1E | 913 | CD1A, R2 |
| CD22 molecule | CD22 | 933 | SIGLEC2; SIGLEC-2 |
| CD226 molecule | CD226 | 10666 | DNAM-1, DNAM1, PTA1, TLiSA1 |
| CD24 molecule | CD24 | 100133941 | CD24A |
| CD244 molecule | CD244 | 51744 | 2B4, NAIL, NKR2B4, Nmrk, SLAMF4 |
| CD27 molecule | CD27 | 939 | T14; S152; Tp55; TNFRSF7; S152. LPFS2 |
| CD207 molecule | CD207 | 50489 | CLEC4K |
| CD274 molecule | CD274 | 29126 | B7-H; B7H1; PDL1; PD-L1; hPD-L1; PDCD1L1; |
| | | | PDCD1LG1 |
| CD276 molecule | CD276 | 80381 | 4Ig-B7-H3, B7-H3, B7H3, B7RP-2 |
| CD28 molecule | CD28 | 940 | Tp44 |
| CD33 molecule | CD33 | 945 | p67; SIGLEC3; SIGLEC-3 |
| CD37 molecule | CD37 | 951 | GP52-40; TSPAN26 |
| CD38 molecule | CD38 | 952 | ADPRC1; ADPRC 1 |
| CD40 ligand | CD40LG | 959 | IGM; IMD3; TRAP; gp39; CD154; CD40L; HIGM1; |
| | | | T-BAM; TNFSF5; hCD40L |
| CD40 molecule | CD40 | 958 | p50; Bp50; CDW40; TNFRSF5 |
| CD44 molecule | CD44 | 960 | IN; LHR; MC56; MDU2; MDU3; MIC4; Pgp1; |
| | | | CDW44; CSPG8; HCELL; HUTCH-I; ECMR-III |
| CD47 molecule | CD47 | 961 | IAP, MER6, OA3 |
| CD48 molecule | CD48 | 962 | BCM1; BLAST; hCD48; mCD48; BLAST1; |
| | | | SLAMF2; MEM-102 |
| CD52 molecule | CD52 | 1043 | HE5; CDW52; EDDM5 |
| CD70 molecule | CD70 | 970 | CD27L; LPFS3; CD27-L; CD27LG; TNFSF7; |
| | | | TNLG8A |
| CD74 molecule | CD74 | 972 | II; p33; DHLAG; HLADG; Ia-GAMMA |
| CD79a molecule | CD79A | 973 | IGA; MB-1 |
| CD79b molecule | CD79B | 974 | B29; IGB; AGM6 |
| CD80 molecule | CD80 | 941 | B7; BB1; B7-1; B7.1; LAB7; CD28LG; CD28LG1 |
| CD84 molecule | CD84 | 8832 | LY9B; hCD84; mCD84; SLAMF5 |
| CD86 molecule | CD86 | 942 | B70; B7-2; B7.2; LAB72; CD28LG2 |
| CD96 molecule | CD96 | 10225 | TACTILE |
| cell adhesion molecule 1 | CADM1 | 23705 | BL2, IGSF4, IGSF4A, NECL2, Necl-2, RA175, ST17, |
| | | | SYNCAM, TSLC1, sTSLC-1, sgIGSF, synCAM1 |
| chorionic somatomammotropin hormone 1 | CSH1 | 1442 | PL; CSA; CS-1; CSMT; GHB3; hCS-1; hCS-A |
| coagulation factor III, tissue factor | F3 | 2152 | TF; TFA; CD142 |
| collagen type IV alpha 1 chain | COL4A1 | 1282 | BSVD, BSVD1, RATOR |
| collagen type IV alpha 2 chain | COL4A2 | 1284 | BSVD2, ICH, POREN2 |
| collagen type IV alpha 3 chain | COL4A3 | 1285 | ATS2, ATS3 |
| collagen type IV alpha 4 chain | COL4A4 | 1286 | ATS2, BFH, CA44 |
| collagen type IV alpha 5 chain | COL4A5 | 1287 | ASLN, ATS, ATS1, CA54 |
| collectin subfamily member 10 | COLEC10 | 10584 | 3MC3; CLL1; CL-34 |
| C-type lectin domain containing 9A | CLEC9A | 283420 | CD370; DNGR1; DNGR-1; UNQ9341 |
| C-type lectin domain family 12 member A | CLEC12A | 160364 | CLL1; MICL; CD371; CLL-1; DCAL-2 |
| C-type lectin domain family 4 member C | CLEC4C | 170482 | DLEC; HECL; BDCA2; CD303; CLECSF7; |
| | | | CLECSF11; PRO34150 |
| C-X-C motif chemokine receptor 1 | CXCR1 | 3577 | C—C, C-C-CKR-1, CD128, CD181, CDw128a, |
| | | | CKR-1, CMKAR1, IL8R1, IL8RA, IL8RBA |
| C-X-C motif chemokine receptor 2 | CXCR2 | 3579 | CD182, CDw128b, CMKAR2, IL8R2, IL8RA, IL8RB |
| C-X-C motif chemokine receptor 3 | CXCR3 | 2833 | CD182, CD183, CKR-L2, CMKAR3, GPR9, IP10-R, |
| | | | Mig-R, MigR |
| C-X-C motif chemokine receptor 4 | CXCR4 | 7852 | CD184, D2S201E, FB22, HM89, HSY3RR, LAP-3, |
| | | | LAP3, LCR1, LESTR, NPY3R, NPYR, NPYRL, |
| | | | NPYY3R, WHIM, WHIMS |
| cytokine inducible SH2 containing protein | CISH | 1154 | CIS; G18; SOCS; CIS-1; BACTS2 |
| cytotoxic T-lymphocyte associated | CTLA4 | 1493 | ALPS5, CD, CD152, CELIAC3, CTLA-4, GRD4, |
| protein 4 | | | GSE, IDDM12 |
| delta like canonical Notch ligand 3 | DLL3 | 10683 | SCDO1 |
| ectonucleotide pyrophosphatase/ | ENPP3 | 5169 | B10; NPP3; PDNP3; CD203c; PD-IBETA |
| phosphodiesterase 3 | | | |
| ectonucleoside triphosphate | ENTPD1 | 953 | CD39; SPG64; ATPDase; NTPDase-1 |
| diphosphohydrolase 1 | | | |
| EPH receptor A1 | EPHA1 | 2041 | EPH; EPHT; EPHT1 |
| EPH receptor A2 | EPHA2 | 1969 | ECK; CTPA; ARCC2; CTPP1; CTRCT6 |
| EPH receptor A4 | EPHA4 | 2043 | EK8; SEK; HEK8; TYRO1 |
| EPH receptor A5 | EPHA5 | 2044 | EK7; CEK7; EHK1; HEK7; EHK-1; TYRO4 |
| EPH receptor A7 | EPHA7 | 2045 | EHK3; EK11; EHK-3; HEK11 |
| ephrin A1 | EFNA1 | 1942 | B61; EFL1; ECKLG; EPLG1; LERK1; LERK-1; |
| | | | TNFAIP4 |
| epidermal growth factor receptor, | EGFR (e.g., | 1956 | ERBB; HER1; mENA; ERBB1; PIG61; NISBD2 |
| including variant III | EGFRvIII) | | |
| epithelial cell adhesion molecule | EPCAM | 4072 | ESA; KSA; M4S1; MK-1; DIAR5; EGP-2; EGP40; |
| | | | KS1/4; MIC18; TROP1; EGP314; HNPCC8; |
| | | | TACSTD1 |
| erb-b2 receptor tyrosine kinase 2 | ERBB2 | 2064 | NEU; NGL; HER2; TKR1; CD340; HER-2; MLN 19; |
| | | | HER-2/neu |
| erb-b2 receptor tyrosine kinase 3 | ERBB3 | 2065 | ErbB-3, FERLK, HER3, LCCS2, MDA-BF-1, |
| | | | c-erbB-3, c-erbB3, erbB3-S, p180-ErbB3, p45-sErbB3, |
| | | | p85-sErbB3 |
| erb-b2 receptor tyrosine kinase 4 | ERBB4 | 2066 | ALS19, HER4, p180erbB4 |
| Fc fragment of IgE receptor Ia | FCER1A | 2205 | FCE1A, FcERI |
| Fc fragment of IgG receptor IIIa | FCGR3A | 2214 | CD16; FCG3; CD16A; FCGR3; IGFR3; IMD20; |
| | | | FCR-10; FCRIII; FCGRIII; FCRIIIA |
| fibroblast activation protein alpha | FAP | 2191 | DPPIV, FAPA, FAPalpha, SIMP |
| fibroblast growth factor receptor 1 | FGFR1 | 2260 | BFGFR, CD331, CEK, ECCL, FGFBR, FGFR-1, FLG, |
| | | | FLT-2, FLT2, HBGFR, HH2, HRTFDS, KAL2, |
| | | | N-SAM, OGD, bFGF-R-1 |
| fibroblast growth factor receptor 2 | FGFR2 | 2263 | BEK; JWS; BBDS; CEK3; CFD1; ECT1; KGFR; |
| | | | TK14; TK25; BFR-1; CD332; K-SAM |
| fibroblast growth factor receptor 3 | FGFR3 | 2261 | ACH, CD333, CEK2, HSFGFR3EX, JTK4 |
| fms related tyrosine kinase 1 | FLT1 | 2321 | FLT, FLT-1, VEGFR-1, VEGFR1 |
| fms related tyrosine kinase 4 | FLT4 | 2324 | FLT-4, FLT41, LMPH1A, LMPHM1, PCL, VEGFR-3, |
| | | | VEGFR3 |
| folate hydrolase 1 | FOLH1 | 2346 | PSM; FGCP; FOLH; GCP2; PSMA; mGCP; GCPII; |
| | | | NAALAD1; NAALAdase, carboxypeptidase II |
| folate receptor 1 | FOLR1 | 2348 | FBP; FOLR, FRα |
| galectin 9 | LGALS9 | 3965 | HUAT, LGALS9A |
| glypican 3 | GPC3 | 2719 | SGB; DGSX; MXR7; SDYS; SGBS; OCI-5; SGBS1; |
| | | | GTR2-2 |
| GPNMB glycoprotein nmb | GPNMB | 10457 | NMB; HGFIN; PLCA3; osteoactivin |
| guanylate cyclase 2C | GUCY2C | 2984 | GC-C; STAR; DIAR6; GUC2C; MECIL; MUCIL |
| hepatitis A virus cellular receptor 2 | HAVCR2 | 84868 | TIM3; CD366; KIM-3; SPTCL; TIMD3; Tim-3; |
| | | | TIMD-3; HAVcr-2 |
| HERV-H LTR-associating 2 | HHLA2 | 11148 | B7-H5, B7-H7, B7H7, B7y |
| immunoglobulin superfamily member 11 | IGSF11 | 152404 | CT119; VSIG3; Igsf13; BT-IgSF; CXADRL1 |
| inducible T cell costimulator | ICOS | 29851 | AILIM, CD278, CVID1 |
| inducible T cell costimulator ligand | ICOSLG | 23308 | B7-H2, B7H2, B7RP-1, B7RP1, B7h, CD275, GL50, |
| | | | ICOS-L, ICOSL, LICOS |
| integrin subunit alpha 5 | ITGA5 | 3678 | CD49e, FNRA, VLA-5, VLA5A |
| integrin subunit alpha V | ITGAV | 3685 | CD51, MSK8, VNRA, VTNR |
| integrin subunit beta 7 | ITGB7 | 3695 | |
| interleukin 2 receptor subunit alpha | IL2RA | 3559 | p55; CD25; IL2R; IMD41; TCGFR; IDDM10 |
| interleukin 3 receptor subunit alpha | IL3RA | 3563 | IL3R; CD123; IL3RX; IL3RY; IL3RAY; hIL-3Ra |
| killer cell immunoglobulin like receptor, | KIR3DL1 | 3811 | CD158E1, KIR, KIR3DL1/S1, NKAT-3, NKAT3, |
| three Ig domains and long cytoplasmic | | | NKB1, NKB1B |
| tail 1 | | | |
| killer cell immunoglobulin like receptor, | KIR2DL1 | 3802 | CD158A, KIR-K64, KIR221, KIR2DL3, NKAT, |
| two Ig domains and long cytoplasmic tail 1 | | | NKAT-1, NKAT1, p58.1 |
| killer cell immunoglobulin like receptor, | KIR2DL2 | 3803 | CD158B1, CD158b, NKAT-6, NKAT6, p58.2 |
| two Ig domains and long cytoplasmic tail 2 | | | |
| killer cell immunoglobulin like receptor, | KIR2DL3 | 3804 | p58; NKAT; GL183; NKAT2; CD158b; KIR2DL; |
| two Ig domains and long cytoplasmic tail 3 | | | NKAT2A; NKAT2B; CD158B2; KIR-K7b; KIR-K7c; |
| | | | KIR2DS5; KIRCL23; KIR-023GB |
| killer cell lectin like receptor C1 | KLRC1 | 3821 | CD159A, NKG2, NKG2A |
| killer cell lectin like receptor C2 | KLRC2 | 3822 | CD159c, NKG2-C, NKG2C |
| killer cell lectin like receptor C3 | KLRC3 | 3823 | NKG2E; NKG2-E |
| killer cell lectin like receptor C4 | KLRC4 | 8302 | NKG2-F, NKG2F |
| killer cell lectin like receptor D1 | KLRD1 | 3824 | CD94 |
| killer cell lectin like receptor G1 | KLRG1 | 10219 | 2F1, CLEC15A, MAFA, MAFA-2F1, MAFA-L, |
| | | | MAFA-LIKE |
| killer cell lectin like receptor K1 | KLRK1 | 22914 | CD314, D12S2489E, KLR, NKG2-D, NKG2D |
| kinase insert domain receptor | KDR | 3791 | CD309, FLK1, VEGFR, VEGFR2 |
| KIT proto-oncogene, receptor tyrosine | KIT | 3815 | PBT; SCFR; C-Kit; CD117; MASTC |
| kinase | | | |
| KRAS proto-oncogene, GTPase | KRAS | 3845 | NS; NS3; CFC2; RALD; K-Ras; KRAS1; KRAS2; |
| | | | RASK2; KI-RAS; C-K-RAS; K-RAS2A; K-RAS2B; |
| | | | K-RAS4A; K-RAS4B; c-Ki-ras2 |
| leukocyte immunoglobulin like receptor B1 | LILRB1 | 10859 | ILT2; LIR1; MIR7; PIRB; CD85J; ILT-2; LIR-1; |
| | | | MIR-7; PIR-B |
| leukocyte immunoglobulin like receptor B2 | LILRB2 | 10288 | ILT4; LIR2; CD85D; ILT-4; LIR-2; MIR10; MIR-10 |
| LY6/PLAUR domain containing 3 | LYPD3 | 27076 | C4.4A |
| lymphocyte activating 3 | LAG3 | 3902 | CD223 |
| lymphocyte antigen 9 | LY9 | 4063 | CD229, SLAMF3, hly9, mLY9 |
| MAGE family member A1 | MAGEA1 | 4100 | CT1.1; MAGE1 |
| MAGE family member A11 | MAGEA11 | 4110 | CT1.11; MAGE11; MAGE-11; MAGEA-11 |
| MAGE family member A3 | MAGEA3 | 4102 | HIP8; HYPD; CT1.3; MAGE3; MAGEA6 |
| MAGE family member A4 | MAGEA4 | 4103 | CT1.4; MAGE4; MAGE4A; MAGE4B; MAGE-41; |
| | | | MAGE-X2 |
| MAGE family member C1 | MAGEC1 | 9947 | CT7; CT7.1 |
| MAGE family member D1 | MAGED1 | 9500 | NRAGE; DLXIN-1 |
| MAGE family member D2 | MAGED2 | 10916 | 11B6; BCG1; BCG-1; HCA10; BARTS5; MAGE-D2 |
| major histocompatibility complex, | HLA-E | 3133 | QA1; HLA-6.2 |
| class I, E | | | |
| major histocompatibility complex, | HLA-F | 3134 | HLAF; CDA12; HLA-5.4; HLA-CDA12 |
| class I, F | | | |
| major histocompatibility complex, | HLA-G | 3135 | MHC-G |
| class I, G | | | |
| membrane spanning 4-domains A1 | MS4A1 | 931 | B1; S7; Bp35; CD20; CVID5; MS4A2; LEU-16 |
| Mesothelin | MSLN | 10232 | MPF, SMRP |
| MET proto-oncogene, receptor tyrosine | MET | 4233 | HGFR; AUTS9; RCCP2; c-Met; DFNB97 |
| kinase | | | |
| MHC class I polypeptide-related | MICA | 100507436 | MIC-A, PERB11.1 |
| sequence A | | | |
| MHC class I polypeptide-related | MICB | 4277 | PERB11.2 |
| sequence B | | | |
| mucin 1, cell surface associated, and splice | MUC1 | 4582 | ADMCKD, ADMCKD1, CA 15-3, CD227, EMA, |
| variants thereof (e.g., including MUC1/A, | | | H23AG, KL-6, MAM6, MCD, MCKD, MCKD1, |
| C, D, X, Y, Z and REP) | | | MUC-1, MUC-1/SEC, MUC-1/X, MUC1/ZD, PEM, |
| | | | PEMT, PUM |
| mucin 16, cell surface associated | MUC16 | 94025 | CA125 |
| natural killer cell cytotoxicity receptor 3 | NCR3LG1 | 374383 | B7-H6, B7H6, DKFZp686O24166 |
| ligand 1 | | | |
| necdin, MAGE family member | NDN | 4692 | PWCR; HsT16328 |
| nectin cell adhesion molecule 2 | NECTIN2 | 5819 | CD112, HVEB, PRR2, PVRL2, PVRR2 |
| nectin cell adhesion molecule 4 | NECTIN4 | 81607 | EDSS1, LNIR, PRR4, PVRL4, nectin-4 |
| neural cell adhesion molecule 1 | NCAM1 | 4684 | CD56, MSK39, NCAM |
| neurophilin 1 | NRP1 | 8829 | NP1; NRP; BDCA4; CD304; VEGF165R |
| Periostin | POSTN | 10631 | OSF-2, OSF2, PDLPOSTN, PN |
| Poliovirus receptor (PVR) cell adhesion | PVR | 5817 | CD155, HVED, NECL5, Necl-5, PVS, TAGE4 |
| molecule | | | |
| programmed cell death 1 | PDCD1 | 5133 | PD1; PD-1; CD279; SLEB2; hPD-1; hPD-1; hSLE1 |
| programmed cell death 1 ligand 2 | PDCD1LG2 | 80380 | B7DC, Btdc, CD273, PD-L2, PDCD1L2, PDL2, |
| | | | bA574F11.2 |
| prominin 1 | PROM1 | 8842 | RP41; AC133; CD133; MCDR2; STGD4; CORD12; |
| | | | PROML1; MSTP061 |
| promyelocytic leukemia | PML | 5371 | MYL, PP8675, RNF71, TRIM19 |
| protein tyrosine kinase 7 (inactive) | PTK7 | 5754 | CCK-4, CCK4 |
| PVR related immunoglobulin domain | PVRIG | 79037 | C7orf15, CD112R |
| containing | | | |
| retinoic acid early transcript 1E | RAET1E | 135250 | LETAL, N2DL-4, NKG2DL4, RAET1E2, RL-4, |
| | | | ULBP4, bA350J20.7 |
| retinoic acid early transcript 1G | RAET1G | 353091 | ULPB5 |
| retinoic acid early transcript 1L | RAET1L | 154064 | ULPB6 |
| roundabout guidance receptor 4 | ROBO4 | 54538 | ECSM4, MRB |
| sialic acid binding Ig like lectin 9 | SIGLEC9 | 27180 | CD329; CDw329; FOAP-9; siglec-9; OBBP-LIKE |
| sialic acid binding Ig like lectin 10 | SIGLEC10 | 89790 | SLG2; PRO940; SIGLEC-10 |
| sialic acid binding Ig like lectin 10 | SIGLEC10 | 89790 | SLG2; PRO940; SIGLEC-10 |
| signal regulatory protein alpha | SIRPA | 140885 | BIT; MFR; P84; SIRP; MYD-1; SHPS1; CD172A; |
| | | | PTPNS1 |
| signaling lymphocytic activation molecule | SLAMF1 | 6504 | SLAM; CD150; CDw150 |
| family member 1 | | | |
| SLAM family member 6 | SLAMF6 | 114836 | CD352, KALI, KALIb, Ly108, NTB-A, NTBA, |
| | | | SF2000 |
| SLAM family member 7 | SLAMF7 | 57823 | 19A, CD319, CRACC, CS1 |
| SLIT and NTRK like family member 6 | SLITRK6 | 84189 | DFNMYP |
| solute carrier family 34 (sodium | SLC34A2 | 10568 | NPTIIb; NAPI-3B; NAPI-IIb |
| phosphate), member 2 | | | |
| solute carrier family 39 member 6 | SLC39A6 | 25800 | LIV-1, ZIP6 |
| solute carrier family 44 member 4 | SLC44A4 | 80736 | C6orf29, CTL4, DFNA72, NG22, TPPT, hTPPT1 |
| STEAP family member 1 | STEAP1 | 26872 | PRSS24, STEAP |
| syndecan 1 | SDC1 | 6382 | SDC; CD138; SYND1; syndecan |
| T cell immunoglobulin and mucin domain | TIMD4 | 91937 | SMUCKLER, TIM4 |
| containing 4 | | | |
| T cell immunoreceptor with Ig and ITIM | TIGIT | 201633 | VSIG9, VSTM3, WUCAM |
| domains | | | |
| tenascin C | TNC | 3371 | 150-225, DFNA56, GMEM, GP, HXB, JI, TN, TN-C |
| thrombomodulin | THBD | 7056 | AHUS6, BDCA3, CD141, THPH12, THRM, TM |
| TNF receptor superfamily member 10a | TNFRSF10A | 8797 | APO2, CD261, DR4, TRAILR-1, TRAILR1 |
| TNF receptor superfamily member 10b | TNFRSF10B | 8795 | CD262, DR5, KILLER, KILLER/DR5, TRAIL-R2, |
| | | | TRAILR2, TRICK2, TRICK2A, TRICK2B, TRICKB, |
| | | | ZTNFR9 |
| TNF receptor superfamily member 14 | TNFRSF14 | 8764 | ATAR, CD270, HVEA, HVEM, LIGHTR, TR2 |
| TNF receptor superfamily member 17 | TNFRSF17 | 608 | BCM, BCMA, CD269, TNFRSF13A |
| TNF receptor superfamily member 18 | TNFRSF18 | 8784 | AITR, CD357, GITR, GITR-D |
| TNF receptor superfamily member 4 | TNFRSF4 | 7293 | OX40; ACT35; CD134; IMD16; TXGP1L |
| TNF receptor superfamily member 8 | TNFRSF8 | 943 | CD30; Ki-1; D1S166E |
| TNF receptor superfamily member 9 | TNFRSF9 | 3604 | 4-1BB, CD137, CDw137, ILA |
| TNF superfamily member 10 | TNFSF10 | 8743 | APO2L, Apo-2L, CD253, TL2, TNLG6A, TRAIL |
| TNF superfamily member 13b | TNFSF13B | 10673 | BAFF, BLYS, CD257, DTL, TALL-1, TALL1, |
| | | | THANK, TNFSF20, TNLG7A, ZTNF4 |
| TNF superfamily member 14 | TNFSF14 | 8740 | CD258, HVEML, LIGHT, LTg |
| TNF superfamily member 18 | TNFSF18 | 8995 | AITRL, GITRL, TL6, TNLG2A, hGITRL |
| TNF superfamily member 4 | TNFSF4 | 7292 | CD134L, CD252, GP34, OX-40L, OX4OL, TNLG2B, |
| | | | TXGP1 |
| TNF superfamily member 8 | TNFSF8 | 944 | CD153, CD30L, CD30LG, TNLG3A |
| TNF superfamily member 9 | TNFSF9 | 8744 | 4-1BB-L, CD137L, TNLG5A |
| transferrin | TF | 7018 | HEL-S-71p, PRO1557, PRO2086, TFQTL1 |
| transforming growth factor beta 1 and | TGFB1 | 7040 | CED, DPD1, IBDIMDE, LAP, TGFB, TGFbeta |
| isoforms thereof | | | |
| transmembrane and immunoglobulin | TMIGD2 | 126259 | CD28H, IGPR-1, IGPR1 |
| domain containing 2 | | | |
| trophinin | TRO | 7216 | MAGE-d3, MAGED3 |
| trophoblast glycoprotein | TPBG | 7162 | 5T4, 5T4AG, M6P1, WAIF1 |
| tumor associated calcium signal | TACSTD2 | 4070 | EGP-1, EGP1, GA733-1, GA7331, GP50, M1S1, |
| transducer 2 | | | TROP2 |
| UL16 binding protein 1 | ULBP1 | 80329 | N2DL-1, NKG2DL1, RAET1I |
| UL16 binding protein 2 | ULBP2 | 80328 | ALCAN-alpha, N2DL2, NKG2DL2, RAET1H, |
| | | | RAET1L |
| UL16 binding protein 3 | ULBP3 | 79465 | N2DL-3, NKG2DL3, RAET1N |
| V-set domain containing T cell activation | VTCN1 | 79679 | B7-H4, B7H4, B7S1, B7X, B7h.5, PRO1291, VCTN1 |
| inhibitor 1 | | | |
| V-set immunoregulatory receptor | VSIR | 64115 | B7-H5, B7H5, C10orf54, DD1alpha, Dies1, GI24, |
| | | | PD-1H, PP2135, SISP1, VISTA |
| X-C motif chemokine receptor 1 | XCR1 | 2829 | GPR5; CCXCR1 |

In some embodiments, the target antigen comprises a tumor-associated carbohydrate antigen (TACA). Illustrative carbohydrate antigen targets include, e.g., mucin TACAs, including truncated glycans Thomsen-nouveau (Tn) (GalNAcα1-Ser/Thr) and STn (Neu5Acα2,6GalNAcα1-Ser/Thr), R1\42 antigen hexasaccharide, carbasugars, C-glycosides, gangliosides GM2, GD2 and GD3; globo-H, sialyl Lewis(a), sialyl Lewis(x) and sialyl Lewis(y) antigens. TACAs are described, e.g., in Sadraei, et al., Adv Carbohydr Chem Biochem. (2017) 74:137-237; Sletmoen, et al., Glycobiology. (2018) 28(7):437-442; Chuang, et al., J Am Chem Soc. (2013) 135(30):11140-50; Ragupathi, Cancer. Immunol Immunother. (1996) 43(3):152-7; Ugorski, et al., Acta Biochim Pol. 2002; 49(2):303-11; Takada, et al., Cancer Res. 1993 Jan. 15; 53(2):354-61.

In some embodiments, the target antigen comprises a neoantigen presented within a major histocompatibility complex (MHC) class I or class II molecule. See, e.g., Ott, et al., Nature. (2017) 547(7662):217-221; Capietto, et al., Curr Opin Immunol. (2017) 46:58-65; Sun, et al., Cancer Lett. (2017) 392:17-25; Khodadoust, et al., Nature. (2017) 543(7647):723-727; Kreiter, et al., Nature. (2015) 520(7549):692-6; Marty, et al., Cell. (2017) 171(6):1272-1283; and Kochin, et al., Oncoimmunology. (2017) 6(4):e1293214 (describing SUV39H2 peptide presented in HLA-A24).

### Conjugated FLT3L-Fc Fusion Proteins

Any of the FLT3L-Fc fusion proteins, or homodimers or heterodimers thereof, disclosed herein may be conjugated. FLT3L-Fc fusion proteins which are bound to various molecules (e.g., labels) including without limitation macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g., ⁹⁰Y, ¹³¹I, ¹²⁵I, ³⁵S, ³H, ¹²¹In, ⁹⁹Tc), fluorescent substances (e.g., fluorescein and rhodamine), fluorescent proteins, luminescent substances (e.g., luminol), Qdots, haptens, enzymes (e.g., glucose oxidase), metal chelates, biotin, avidin, and drugs.

The above-described conjugated FLT3L-Fc fusion proteins can be prepared according to known methods, e.g., performing chemical modifications on the FLT3L-Fc fusion proteins described herein. In certain embodiments, the labelling moiety or therapeutic moiety is conjugated to the Fc portion of the fusion protein. Methods for modifying antibody Fc regions are well known in the art (e.g., U.S. Pat. Nos. 5,057,313 and 5,156,840).

In some embodiments, the FLT3L-Fc fusion protein, or homodimer or heterodimer thereof, is conjugated to a drug or therapeutic agent. In various embodiments, the drug is a small organic compound or an inhibitory nucleic acid, e.g., a short-inhibitory RNA (siRNA), a microRNA (miRNA). In some embodiments, the drug or therapeutic agent is an anti-neoplastic agent or a chemotherapeutic agent, as known in the art and described herein. In a particular embodiment, the drug or therapeutic agent is selected from the group consisting of monomethyl auristatin E (MIME), monomethyl auristatin F (MMAF), a calicheamicin, ansamitocin, maytansine or an analog thereof (e.g., mertansine/emtansine (DM1), ravtansine/soravtansine (DM4)), an anthracyline (e.g., doxorubicin, daunorubicin, epirubicin, idarubicin), pyrrolobenzodiazepine (PBD) DNA cross-linking agent SC-DR002 (D6.5), duocarmycin, a microtubule inhibitors (MTI) (e.g., a taxane, a vinca alkaloid, an epothilone), a pyrrolobenzodiazepine (PBD) or dimer thereof, and a duocarmycin (A, B1, B2, C1, C2, D, SA, CC-1065).

3. Polynucleotides Encoding FLT3L-Fc Fusion Proteins

Provided are polynucleotides encoding the FLT3L-Fc fusion proteins, described herein, vectors comprising such polynucleotides, and host cells (e.g., human cells, mammalian cells, yeast cells, plant cells, insect cells, bacterial cells, e.g., E. coli) comprising such polynucleotides or expression vectors. Provided herein are polynucleotides comprising nucleotide sequence(s) encoding any of the FLT3L-Fc fusion proteins provided herein, as well as expression cassettes and vector(s) comprising such polynucleotide sequences, e.g., expression vectors for their efficient expression in host cells, e.g., mammalian cells. In various embodiments, the polynucleotide is a DNA, a cDNA, or an mRNA.

The terms “polynucleotide” and “nucleic acid molecule” interchangeably refer to a polymeric form of nucleotides and includes both sense and anti-sense strands of RNA, cDNA, genomic DNA, and synthetic forms and mixed polymers of the above. As used herein, the term nucleic acid molecule may be interchangeable with the term polynucleotide. In some embodiments, a nucleotide refers to a ribonucleotide, deoxynucleotide or a modified form of either type of nucleotide, and combinations thereof. The terms also include without limitation, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-biased polynucleotides for improved expression in a desired host cell.

A “substitution,” as used herein, denotes the replacement of one or more amino acids or nucleotides by different amino acids or nucleotides, respectively.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location. “Isolated nucleic acid encoding an FLT3L-Fc fusion protein” refers to one or more nucleic acid molecules encoding first antigen binding domain, and optionally second antigen binding domain, antibody heavy and light chains (or fragments thereof), including such nucleic acid molecule(s) in a single vector or separate vectors, and such nucleic acid molecule(s) present at one or more locations in a host cell.

An “isolated” polypeptide, such as an isolated FLT3L-Fc fusion protein provided herein, is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In some embodiments, the isolated polypeptide will be purified (1) to greater than 95% by weight of polypeptide as determined by the Lowry method, for example, more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. Isolated polypeptide includes the polypeptide in situ within recombinant cells since at least one component of the antibody's natural environment will not be present. Ordinarily, however, isolated polypeptide will be prepared by at least one purification step.

A “polynucleotide variant,” as the term is used herein, is a polynucleotide that typically differs from a polynucleotide specifically disclosed herein in one or more substitutions, deletions, additions and/or insertions. Such variants may be naturally occurring or may be synthetically generated, for example, by modifying one or more of the polynucleotide sequences described herein and evaluating one or more biological activities of the encoded polypeptide as described herein and/or using any of a number of techniques well known in the art.

In some embodiments, the nucleic acid molecule is codon-biased to enhance expression in a desired host cell, e.g., in human cells, mammalian cells, yeast cells, plant cells, insect cells, or bacterial cells, e.g., E. coli cells. Accordingly, provided are polynucleotides encoding a FLT3L-Fc fusion protein wherein the polynucleotides are codon-biased, comprise replacement heterologous signal sequences, and/or have mRNA instability elements eliminated. Methods to generate codon-biased nucleic acids can be carried out by adapting the methods described in, e.g., U.S. Pat. Nos. 5,965,726; 6,174,666; 6,291,664; 6,414,132; and 6,794,498. Preferred codon usage for expression of the FLT3L-Fc fusion proteins in desired host cells is provided, e.g., at kazusa.or.jp/codon/; and genscript.com/tools/codon-frequency-table.

In some embodiments, the polynucleotide encoding a FLT3L-Fc fusion protein, as described herein, has at least 80%, at least 85%, at least 90%, at least 91%, at least 92%, at least 93%, at least 94%, at least 95%, at least 96%, at least 97%, at least 98%, at least 99% identical, or 100% identical to an nucleic acid sequence selected from the group consisting of SEQ ID NOs: 28-70, as provided in Table C.

As appropriate, in certain embodiments, the 3′-end of the polynucleotide encoding the FLT3L-Fc fusion protein comprises multiple tandem stop codons, e.g., two or more tandem TAG (“amber”), TAA (“ochre”) or TGA (“opal” or “umber”) stop codons. The multiple tandem stop codons can be the same or different.

TABLE C
Polynucleotides encoding FLT3L-Fc fusion proteins

| POLYNUCL. | | |
|---|---|---|
| NO. | | |
| SEQ ID NO: | Features | Polynucleotide Sequence |
| 28 | FLT3L ECD- | ACACAGGATTGCAGCTTCCAGCACAGCCCCATCAGCAGCGATTTCGCCGTGAAGATCAGA |
| | hingeless hG1 | GAGCTGAGCGACTACCTGCTGCAGGACTACCCTGTGACCGTGGCCAGCAATCTGCAGGAC |
| | | GAAGAACTGTGTGGTGGACTGTGGCGACTGGTGCTGGCTCAGAGATGGATGGAACGGCTG |
| | | AAAACAGTGGCCGGCAGCAAGATGCAGGGACTGCTGGAAAGAGTGAACACCGAGATCCA |
| | | CTTCGTGACCAAGTGCGCCTTCCAGCCTCCTCCATCCTGCCTGAGATTCGTGCAGACCAAC |
| | | ATCAGCAGACTGCTGCAAGAGACAAGCGAGCAGCTGGTGGCCCTGAAGCCTTGGATCACC |
| | | AGACAGAACTTCAGCCGGTGCCTGGAACTGCAGTGTCAGCCCGATAGCAGCACACTGCCTC |
| | | CGCCTTGGAGTCCTAGACCTCTGGAAGCCACAGCTCCCACCGCTCCTCAAGGCGGACCGTC |
| | | AGTCTTCCTCTTCCCCCCAAAACCCAAGGACACCCTCATGATCTCCCGGACCCCTGAGGTC |
| | | ACATGCGTGGTGGTGGACGTGAGCCACGAAGACCCTGAGGTCAAGTTCAACTGGTACGTG |
| | | GACGGCGTGGAGGTGCATAATGCCAAGACAAAGCCGCGGGAGGAGCAGTACAACAGCAC |
| | | GTACCGTGTGGTCAGCGTCCTCACCGTCCTGCACCAGGACTGGCTGAATGGCAAGGAGTAC |
| | | AAGTGCAAGGTCTCCAACAAAGCCCTCCCAGCCCCCATCGAGAAAACCATCTCCAAAGCC |
| | | AAAGGGCAGCCCCGAGAACCACAGGTGTACACCCTGCCCCCATCCCGGGAGGAGATGACC |
| | | AAGAACCAGGTCAGCCTGACCTGCCTGGTCAAAGGCTTCTATCCCAGCGACATCGCCGTGG |
| | | AGTGGGAGAGCAATGGGCAGCCGGAGAACAACTACAAGACCACGCCTCCCGTGCTGGACT |
| | | CCGACGGCTCCTTCTTCCTCTACAGCAAGCTCACCGTGGACAAGAGCAGGTGGCAGCAGGG |
| | | GAACGTCTTCTCATGCTCCGTGATGCATGAGGCTCTGCACAACCACTACACGCAGAAGAGC |
| | | CTCTCCCTGTCTCCGGGTAAA |
| 29 | FLT3L ECD- | ACCCAGGACTGCTCCTTCCAGCACTCCCCTATCTCTTCCGACTTCGCCGTGAAGATCAGAG |
| | hingeless hG1 | AGCTGTCCGACTACCTGCTGCAGGACTACCCTGTGACCGTGGCCAGCAATCTGCAGGACGA |
| | | AGAACTGTGTGGTGGCCTGTGGCGACTGGTGTTGGCTCAGAGATGGATGGAACGGCTGAA |
| | | AACCGTGGCCGGCTCTAAGATGCAGGGCCTGCTGGAAAGAGTGAACACCGAGATCCACTT |
| | | CGTGACCAAGTGCGCCTTTCAGCCTCCTCCATCCTGCCTGAGATTCGTGCAGACCAACATCT |
| | | CCCGGCTGCTGCAAGAGACATCCGAGCAGCTGGTGGCTCTGAAGCCCTGGATCACCCGGC |
| | | AGAACTTCTCTCGGTGTCTGGAACTGCAGTGTCAGCCCGACTCTTCTACCCTGCCTCCACCT |
| | | TGGAGCCCCAGACCTTTGGAAGCTACCGCTCCAACAGCTCCTCAAGGCGGCCCTTCCGTGT |
| | | TTCTGTTCCCTCCAAAGCCTAAGGACACCCTGATGATCTCTCGGACCCCTGAAGTGACCTG |
| | | CGTGGTGGTGGATGTGTCTCACGAGGATCCCGAAGTGAAGTTCAATTGGTACGTGGACGGC |
| | | GTGGAAGTGCACAACGCCAAGACCAAGCCTAGAGAGGAACAGTACAACTCCACCTACAGA |
| | | GTGGTGTCCGTGCTGACCGTGCTGCACCAGGATTGGCTGAACGGCAAAGAGTACAAGTGC |
| | | AAGGTGTCCAACAAGGCCCTGCCTGCTCCTATCGAAAAGACCATCTCCAAGGCCAAGGGC |
| | | CAGCCTAGGGAACCCCAGGTTTACACCCTGCCACCTAGCCGGGAAGAGATGACCAAGAAC |
| | | CAGGTGTCCCTGACCTGCCTGGTCAAGGGCTTCTACCCCTCTGATATCGCCGTGGAATGGG |
| | | AGAGCAATGGCCAGCCTGAGAACAACTACAAGACCACACCTCCTGTGCTGGACTCCGACG |
| | | GCTCATTCTTCCTGTACTCCAAGCTGACAGTGGACAAGTCCAGATGGCAGCAGGGCAACGT |
| | | GTTCTCCTGCTCCGTGATGCACGAGGCTCTGCACAACCACTACACCCAGAAGTCCCTGTCT |
| | | CTGTCCCCTGGCAAA |
| 30 | FLT3L ECD (Δ | ACACAGGATTGCAGCTTCCAGCACAGCCCCATCAGCAGCGATTTCGCCGTGAAGATCAGA |
| | C-term 5aa (PTAPQ; | GAGCTGAGCGACTACCTGCTGCAGGACTACCCTGTGACCGTGGCCAGCAATCTGCAGGAC |
| | SEQ ID NO: 85)- | GAAGAACTGTGTGGTGGACTGTGGCGACTGGTGCTGGCTCAGAGATGGATGGAACGGCTG |
| | hingeless hG1 | AAAACAGTGGCCGGCAGCAAGATGCAGGGACTGCTGGAAAGAGTGAACACCGAGATCCA |
| | | CTTCGTGACCAAGTGCGCCTTCCAGCCTCCTCCATCCTGCCTGAGATTCGTGCAGACCAAC |
| | | ATCAGCAGACTGCTGCAAGAGACAAGCGAGCAGCTGGTGGCCCTGAAGCCTTGGATCACC |
| | | AGACAGAACTTCAGCCGGTGCCTGGAACTGCAGTGTCAGCCCGATAGCAGCACACTGCCTC |
| | | CGCCTTGGAGTCCTAGACCTCTGGAAGCCACAGCTGGGGGACCGTCAGTCTTCCTCTTCCC |
| | | CCCAAAACCCAAGGACACCCTCATGATCTCCCGGACCCCTGAGGTCACATGCGTGGTGGTG |
| | | GACGTGAGCCACGAAGACCCTGAGGTCAAGTTCAACTGGTACGTGGACGGCGTGGAGGTG |
| | | CATAATGCCAAGACAAAGCCGCGGGAGGAGCAGTACAACAGCACGTACCGTGTGGTCAGC |
| | | GTCCTCACCGTCCTGCACCAGGACTGGCTGAATGGCAAGGAGTACAAGTGCAAGGTCTCCA |
| | | ACAAAGCCCTCCCAGCCCCCATCGAGAAAACCATCTCCAAAGCCAAAGGGCAGCCCCGAG |
| | | AACCACAGGTGTACACCCTGCCCCCATCCCGGGAGGAGATGACCAAGAACCAGGTCAGCC |
| | | TGACCTGCCTGGTCAAAGGCTTCTATCCCAGCGACATCGCCGTGGAGTGGGAGAGCAATGG |
| | | GCAGCCGGAGAACAACTACAAGACCACGCCTCCCGTGCTGGACTCCGACGGCTCCTTCTTC |
| | | CTCTACAGCAAGCTCACCGTGGACAAGAGCAGGTGGCAGCAGGGGAACGTCTTCTCATGC |
| | | TCCGTGATGCATGAGGCTCTGCACAACCACTACACGCAGAAGAGCCTCTCCCTGTCTCCGG |
| | | GTAAA |
| 31 | FLT3L ECD (Δ | ACCCAGGACTGCTCCTTCCAGCACTCCCCTATCTCTTCCGACTTCGCCGTGAAGATCAGAG |
| | C-term 5aa (PTAPQ; | AGCTGTCCGACTACCTGCTGCAGGACTATCCTGTGACCGTGGCCAGCAACCTGCAGGATGA |
| | SEQ ID NO: 85)- | AGAACTGTGTGGTGGACTGTGGCGACTGGTGCTGGCTCAGAGATGGATGGAAAGACTGAA |
| | hingeless hG1 | AACCGTGGCCGGCTCCAAGATGCAGGGACTGCTGGAAAGAGTGAACACAGAGATCCACTT |
| | | CGTGACCAAGTGCGCCTTTCAGCCTCCTCCATCCTGCCTGAGATTCGTGCAGACCAACATCT |
| | | CCCGGCTGCTGCAAGAGACATCTGAGCAGCTGGTGGCCCTGAAGCCTTGGATCACCCGGCA |
| | | GAACTTCTCTCGGTGCCTGGAACTGCAGTGTCAGCCTGATTCTTCTACCCTGCCTCCACCTT |
| | | GGAGCCCTAGACCTTTGGAAGCTACAGCTGGCGGCCCAAGCGTGTTCCTGTTTCCTCCAAA |
| | | GCCTAAGGACACCCTGATGATCTCTCGGACCCCTGAAGTGACCTGCGTGGTGGTGGATGTG |
| | | TCTCACGAGGATCCCGAAGTGAAGTTCAATTGGTACGTGGACGGCGTGGAAGTGCACAAC |
| | | GCCAAGACCAAGCCTAGAGAGGAACAGTACAACTCCACCTACAGAGTGGTGTCCGTGCTG |
| | | ACCGTGCTGCACCAGGATTGGCTGAACGGCAAAGAGTACAAGTGCAAGGTGTCCAACAAG |
| | | GCCCTGCCTGCTCCTATCGAAAAGACCATCTCCAAGGCCAAGGGCCAGCCTAGGGAACCTC |
| | | AGGTTTACACCCTGCCACCTAGCCGGGAAGAGATGACCAAAAACCAGGTGTCCCTGACCT |
| | | GCCTGGTCAAGGGCTTCTACCCATCCGATATCGCCGTGGAATGGGAGTCTAACGGCCAGCC |
| | | TGAGAACAACTACAAGACCACACCTCCTGTGCTGGACTCCGACGGCTCATTCTTCCTGTAC |
| | | TCCAAGCTGACAGTGGACAAGTCTCGGTGGCAGCAGGGCAACGTGTTCTCCTGTTCTGTGA |
| | | TGCACGAGGCCCTGCACAACCACTACACCCAGAAGTCCCTGTCTCTGTCCCCTGGCAAA |
| 32 | FLT3L ECD - hG4 | ACACAGGATTGCAGCTTCCAGCACAGCCCCATCAGCAGCGATTTCGCCGTGAAGATCAGA |
| | S228P/L235E | GAGCTGAGCGACTACCTGCTGCAGGACTACCCTGTGACCGTGGCCAGCAATCTGCAGGAC |
| | | GAAGAACTGTGTGGTGGACTGTGGCGACTGGTGCTGGCTCAGAGATGGATGGAACGGCTG |
| | | AAAACAGTGGCCGGCAGCAAGATGCAGGGACTGCTGGAAAGAGTGAACACCGAGATCCA |
| | | CTTCGTGACCAAGTGCGCCTTCCAGCCTCCTCCATCCTGCCTGAGATTCGTGCAGACCAAC |
| | | ATCAGCAGACTGCTGCAAGAGACAAGCGAGCAGCTGGTGGCCCTGAAGCCTTGGATCACC |
| | | AGACAGAACTTCAGCCGGTGCCTGGAACTGCAGTGTCAGCCCGATAGCAGCACACTGCCTC |
| | | CGCCTTGGAGTCCTAGACCTCTGGAAGCCACAGCTCCCACCGCTCCTCAAGAATCTAAGTA |
| | | CGGCCCTCCCTGCCCTCCTTGCCCAGCCCCTGAATTTGAGGGCGGACCCTCCGTGTTCCTGT |
| | | TCCCCCCAAAGCCCAAGGACACCCTGATGATCAGCCGGACCCCCGAAGTGACCTGCGTGGT |
| | | GGTGGATGTGTCCCAGGAAGATCCCGAGGTGCAGTTCAATTGGTACGTGGACGGCGTGGA |
| | | AGTGCACAACGCCAAGACCAAGCCCAGAGAGGAACAGTTCAACAGCACCTACCGGGTGGT |
| | | GTCCGTGCTGACAGTGCTGCACCAGGACTGGCTGAACGGCAAAGAGTACAAGTGCAAGGT |
| | | GTCCAACAAGGGCCTGCCCAGCTCCATCGAGAAAACCATCAGCAAGGCCAAGGGCCAGCC |
| | | CCGCGAACCCCAGGTGTACACACTGCCTCCAAGCCAGGAAGAGATGACCAAGAACCAGGT |
| | | GTCCCTGACCTGTCTCGTGAAAGGCTTCTACCCCTCCGATATCGCCGTGGAATGGGAGAGC |
| | | AACGGCCAGCCCGAGAACAACTACAAGACCACCCCCCCTGTGCTGGACAGCGACGGCTCA |
| | | TTCTTCCTGTACAGCAGACTGACCGTGGACAAGAGCCGGTGGCAGGAAGGCAACGTGTTC |
| | | AGCTGCAGCGTGATGCACGAGGCCCTGCACAACCACTACACCCAGAAGTCCCTGTCTCTGT |
| | | CCCTGGGCAAA |
| 33 | FLT3L ECD - hG4 | ACCCAGGACTGCTCCTTCCAGCACTCCCCTATCTCTTCCGACTTCGCCGTGAAGATCAGAG |
| | S228P/L235E | AGCTGTCCGACTACCTGCTGCAGGACTATCCTGTGACCGTGGCCAGCAACCTGCAGGATGA |
| | | AGAACTGTGTGGTGGACTGTGGCGACTGGTGCTGGCTCAGAGATGGATGGAAAGACTGAA |
| | | AACCGTGGCCGGCTCCAAGATGCAGGGACTGCTGGAAAGAGTGAACACAGAGATCCACTT |
| | | CGTGACCAAGTGCGCCTTTCAGCCTCCTCCATCCTGCCTGAGATTCGTGCAGACCAACATCT |
| | | CCCGGCTGCTGCAAGAGACATCTGAGCAGCTGGTGGCCCTGAAGCCTTGGATCACCCGGCA |
| | | GAACTTCTCTCGGTGCCTGGAACTGCAGTGTCAGCCTGATTCTTCTACCCTGCCTCCACCTT |
| | | GGAGCCCTAGACCTTTGGAGGCTACAGCTCCTACCGCTCCTCAAGAGTCTAAGTACGGCCC |
| | | TCCTTGTCCTCCATGTCCTGCTCCAGAATTTGAAGGCGGCCCAAGCGTGTTCCTGTTTCCTC |
| | | CAAAGCCTAAGGACACCCTGATGATCTCTCGGACCCCTGAAGTGACCTGCGTGGTGGTGGA |
| | | TGTGTCTCAAGAGGACCCCGAGGTGCAGTTCAATTGGTACGTGGACGGCGTGGAAGTGCA |
| | | CAACGCCAAGACCAAGCCTAGAGAGGAACAGTTCAACTCCACCTACAGAGTGGTGTCCGT |
| | | GCTGACCGTGCTGCACCAGGATTGGCTGAACGGCAAAGAGTACAAGTGCAAGGTGTCCAA |
| | | CAAGGGCCTGCCTAGCTCCATCGAAAAGACCATCTCCAAGGCCAAGGGCCAGCCAAGAGA |
| | | ACCTCAGGTGTACACACTGCCTCCAAGCCAAGAGGAAATGACCAAGAACCAGGTGTCCCT |
| | | GACCTGCCTGGTCAAGGGCTTCTACCCATCCGATATCGCCGTGGAATGGGAGTCTAACGGC |
| | | CAGCCTGAGAACAACTACAAGACCACACCTCCTGTGCTGGACTCCGACGGCTCCTTCTTTC |
| | | TGTACTCTCGCCTGACCGTGGACAAGTCTAGATGGCAAGAGGGCAACGTGTTCTCCTGCTC |
| | | TGTGATGCACGAGGCCCTGCACAACCACTACACCCAGAAGTCCCTGTCTCTGTCCCTGGGC |
| | | AAA |
| 34 | FLT3L ECD - hG4 | ACACAGGATTGCAGCTTCCAGCACAGCCCCATCAGCAGCGATTTCGCCGTGAAGATCAGA |
| | S228P/ | GAGCTGAGCGACTACCTGCTGCAGGACTACCCTGTGACCGTGGCCAGCAATCTGCAGGAC |
| | F234A/ | GAAGAACTGTGTGGTGGACTGTGGCGACTGGTGCTGGCTCAGAGATGGATGGAACGGCTG |
| | L235A | AAAACAGTGGCCGGCAGCAAGATGCAGGGACTGCTGGAAAGAGTGAACACCGAGATCCA |
| | | CTTCGTGACCAAGTGCGCCTTCCAGCCTCCTCCATCCTGCCTGAGATTCGTGCAGACCAAC |
| | | ATCAGCAGACTGCTGCAAGAGACAAGCGAGCAGCTGGTGGCCCTGAAGCCTTGGATCACC |
| | | AGACAGAACTTCAGCCGGTGCCTGGAACTGCAGTGTCAGCCCGATAGCAGCACACTGCCTC |
| | | CGCCTTGGAGTCCTAGACCTCTGGAAGCCACAGCTCCCACCGCTCCTCAAGAGTCTAAGTA |
| | | CGGCCCTCCTTGTCCTCCATGTCCTGCTCCAGAAGCTGCTGGCGGCCCTTCCGTGTTTCTGT |
| | | TCCCTCCAAAGCCTAAGGACACCCTGATGATCTCTCGGACCCCTGAAGTGACCTGCGTGGT |
| | | GGTGGATGTGTCCCAAGAGGATCCCGAGGTGCAGTTCAATTGGTACGTGGACGGCGTGGA |
| | | AGTGCACAACGCCAAGACCAAGCCTAGAGAGGAACAGTTCAACTCCACCTACAGAGTGGT |
| | | GTCCGTGCTGACCGTGCTGCACCAGGATTGGCTGAACGGCAAAGAGTACAAGTGCAAGGT |
| | | GTCCAACAAGGGCCTGCCTTCCAGCATCGAAAAGACCATCTCCAAGGCCAAGGGCCAGCC |
| | | TAGGGAACCCCAGGTTTACACCCTGCCTCCAAGCCAAGAGGAAATGACCAAGAACCAGGT |
| | | GTCCCTGACCTGCCTGGTCAAGGGCTTCTACCCTTCCGATATCGCCGTGGAATGGGAGAGC |
| | | AATGGCCAGCCTGAGAACAACTACAAGACCACACCTCCTGTGCTGGACTCCGACGGCTCCT |
| | | TCTTTCTGTACTCCCGCCTGACCGTGGACAAGTCCAGATGGCAAGAGGGCAACGTGTTCTC |
| | | CTGCTCCGTGATGCACGAGGCCCTGCACAATCACTACACCCAGAAGTCCCTGTCTCTGTCC |
| | | CTGGGCAAA |
| 35 | FLT3L ECD - hG4 | ACCCAGGACTGCTCCTTCCAGCACTCCCCTATCTCTTCCGACTTCGCCGTGAAGATCAGAG |
| | S228P/ | AGCTGTCCGACTACCTGCTGCAGGACTATCCTGTGACCGTGGCCAGCAACCTGCAGGATGA |
| | F234A/ | AGAACTGTGTGGTGGACTGTGGCGACTGGTGCTGGCTCAGAGATGGATGGAAAGACTGAA |
| | L235A | AACCGTGGCCGGCTCCAAGATGCAGGGACTGCTGGAAAGAGTGAACACAGAGATCCACTT |
| | | CGTGACCAAGTGCGCCTTTCAGCCTCCTCCATCCTGCCTGAGATTCGTGCAGACCAACATCT |
| | | CCCGGCTGCTGCAAGAGACATCTGAGCAGCTGGTGGCCCTGAAGCCTTGGATCACCCGGCA |
| | | GAACTTCTCTCGGTGCCTGGAACTGCAGTGTCAGCCTGATTCTTCTACCCTGCCTCCACCTT |
| | | GGAGCCCTAGACCTTTGGAGGCTACAGCTCCTACCGCTCCTCAAGAGTCTAAGTACGGCCC |
| | | TCCTTGTCCTCCATGTCCTGCTCCAGAAGCTGCTGGCGGACCAAGCGTTTTCCTGTTTCCTC |
| | | CAAAGCCTAAGGACACCCTGATGATCTCTCGGACCCCTGAAGTGACCTGCGTGGTGGTGGA |
| | | TGTGTCTCAAGAGGACCCCGAGGTGCAGTTCAATTGGTACGTGGACGGCGTGGAAGTGCA |
| | | CAACGCCAAGACCAAGCCTAGAGAGGAACAGTTCAACTCCACCTACAGAGTGGTGTCCGT |
| | | GCTGACCGTGCTGCACCAGGATTGGCTGAACGGCAAAGAGTACAAGTGCAAGGTGTCCAA |
| | | CAAGGGCCTGCCTAGCTCCATCGAAAAGACCATCTCCAAGGCCAAGGGCCAGCCAAGAGA |
| | | ACCTCAGGTGTACACACTGCCTCCAAGCCAAGAGGAAATGACCAAGAACCAGGTGTCCCT |
| | | GACCTGCCTGGTCAAGGGCTTCTACCCATCCGATATCGCCGTGGAATGGGAGTCTAACGGC |
| | | CAGCCTGAGAACAACTACAAGACCACACCTCCTGTGCTGGACTCCGACGGCTCCTTCTTTC |
| | | TGTACTCTCGCCTGACCGTGGACAAGTCTAGATGGCAAGAGGGCAACGTGTTCTCCTGCTC |
| | | TGTGATGCACGAGGCCCTGCACAACCACTACACCCAGAAGTCCCTGTCTCTGTCCCTGGGC |
| | | AAA |
| 36 | Aglyco-FLT3L | ATGACAGTTTTGGCTCCAGCTTGGTCCCCTACAACCTACCTGCTGCTGCTGTTGCTGCTCTC |
| | (S128A/S151A) | CTCTGGCCTGTCTGGCACCCAGGACTGTTCCTTCCAGCACTCCCCTATCTCCAGCGACTTCG |
| | hingeless hG1 | CCGTGAAGATCAGAGAGCTGTCCGACTATCTGCTGCAGGACTACCCTGTGACCGTGGCCAG |
| | | CAATCTGCAGGACGAAGAACTGTGTGGTGGCCTGTGGCGACTGGTGTTGGCTCAGAGATG |
| | | GATGGAACGGCTGAAAACCGTGGCCGGCTCTAAGATGCAGGGCCTGCTGGAAAGAGTGAA |
| | | CACCGAGATCCACTTCGTGACCAAGTGCGCCTTTCAGCCTCCTCCATCCTGCCTGAGATTCG |
| | | TGCAGACCAATATCGCCCGGCTGCTGCAAGAGACATCCGAGCAGCTGGTGGCTCTGAAGC |
| | | CCTGGATCACCAGACAGAACTTCGCCCGGTGTCTGGAACTGCAGTGTCAGCCTGACAGCTC |
| | | TACCCTGCCTCCACCTTGGAGCCCTAGACCTCTGGAAGCTACCGCTCCAACCGCTCCTCAA |
| | | GGGGGACCGTCAGTCTTCCTCTTCCCCCCAAAACCCAAGGACACCCTCATGATCTCCCGGA |
| | | CCCCTGAGGTCACATGCGTGGTGGTGGACGTGAGCCACGAAGACCCTGAGGTCAAGTTCA |
| | | ACTGGTACGTGGACGGCGTGGAGGTGCATAATGCCAAGACAAAGCCGCGGGAGGAGCAGT |
| | | ACAACAGCACGTACCGTGTGGTCAGCGTCCTCACCGTCCTGCACCAGGACTGGCTGAATGG |
| | | CAAGGAGTACAAGTGCAAGGTCTCCAACAAAGCCCTCCCAGCCCCCATCGAGAAAACCAT |
| | | CTCCAAAGCCAAAGGGCAGCCCCGAGAACCACAGGTGTACACCCTGCCCCCATCCCGGGA |
| | | GGAGATGACCAAGAACCAGGTCAGCCTGACCTGCCTGGTCAAAGGCTTCTATCCCAGCGA |
| | | CATCGCCGTGGAGTGGGAGAGCAATGGGCAGCCGGAGAACAACTACAAGACCACGCCTCC |
| | | CGTGCTGGACTCCGACGGCTCCTTCTTCCTCTACAGCAAGCTCACCGTGGACAAGAGCAGG |
| | | TGGCAGCAGGGGAACGTCTTCTCATGCTCCGTGATGCATGAGGCTCTGCACAACCACTACA |
| | | CGCAGAAGAGCCTCTCCCTGTCTCCGGGTAAA |
| 37 | Aglyco-FLT3L | ACCCAGGACTGCTCCTTCCAGCACTCCCCTATCTCTTCCGACTTCGCCGTGAAGATCAGAG |
| | (S128A/S151A) | AGCTGTCCGACTACCTGCTGCAGGACTATCCTGTGACCGTGGCCAGCAACCTGCAGGATGA |
| | hingeless hG1 | AGAACTGTGTGGTGGACTGTGGCGACTGGTGCTGGCTCAGAGATGGATGGAAAGACTGAA |
| | | AACCGTGGCCGGCTCCAAGATGCAGGGACTGCTGGAAAGAGTGAACACAGAGATCCACTT |
| | | CGTGACCAAGTGCGCCTTTCAGCCTCCTCCATCCTGCCTGAGATTCGTGCAGACCAATATC |
| | | GCCCGGCTGCTGCAAGAGACATCTGAGCAGCTGGTGGCCCTGAAGCCTTGGATCACCAGA |
| | | CAGAACTTCGCCCGGTGCCTGGAACTGCAGTGTCAGCCTGATTCTTCTACCCTGCCTCCACC |
| | | TTGGAGCCCTAGACCTTTGGAGGCTACAGCTCCTACTGCTCCTCAAGGCGGCCCAAGCGTT |
| | | TTCCTGTTTCCTCCAAAGCCTAAGGACACCCTGATGATCTCTCGGACCCCTGAAGTGACCTG |
| | | CGTGGTGGTGGATGTGTCTCACGAGGATCCCGAAGTGAAGTTCAATTGGTACGTGGACGGC |
| | | GTGGAAGTGCACAACGCCAAGACCAAGCCTAGAGAGGAACAGTACAACTCCACCTACAGA |
| | | GTGGTGTCCGTGCTGACCGTGCTGCACCAGGATTGGCTGAACGGCAAAGAGTACAAGTGC |
| | | AAGGTGTCCAACAAGGCCCTGCCTGCTCCTATCGAAAAGACCATCTCCAAGGCCAAGGGC |
| | | CAGCCTAGGGAACCTCAGGTTTACACCCTGCCACCTAGCCGGGAAGAGATGACCAAAAAC |
| | | CAGGTGTCCCTGACCTGCCTGGTCAAGGGCTTCTACCCATCCGATATCGCCGTGGAATGGG |
| | | AGTCTAACGGCCAGCCTGAGAACAACTACAAGACCACACCTCCTGTGCTGGACTCCGACG |
| | | GCTCATTCTTCCTGTACTCCAAGCTGACAGTGGACAAGTCTCGGTGGCAGCAGGGCAACGT |
| | | GTTCTCCTGTTCTGTGATGCACGAGGCCCTGCACAACCACTACACCCAGAAGTCCCTGTCTC |
| | | TGTCCCCTGGCAAA |
| 38 | FLT3L (Δ C-term | ACACAGGATTGCAGCTTCCAGCACAGCCCCATCAGCAGCGATTTCGCCGTGAAGATCAGA |
| | 5aa (PTAPQ; SEQ | GAGCTGAGCGACTACCTGCTGCAGGACTACCCTGTGACCGTGGCCAGCAATCTGCAGGAC |
| | ID NO: 85))-hG4 | GAAGAACTGTGTGGTGGACTGTGGCGACTGGTGCTGGCTCAGAGATGGATGGAACGGCTG |
| | S228P/ | AAAACAGTGGCCGGCAGCAAGATGCAGGGACTGCTGGAAAGAGTGAACACCGAGATCCA |
| | F234A/ | CTTCGTGACCAAGTGCGCCTTCCAGCCTCCTCCATCCTGCCTGAGATTCGTGCAGACCAAC |
| | L235A | ATCAGCAGACTGCTGCAAGAGACAAGCGAGCAGCTGGTGGCCCTGAAGCCTTGGATCACC |
| | | AGACAGAACTTCAGCCGGTGCCTGGAACTGCAGTGTCAGCCCGATAGCAGCACACTGCCTC |
| | | CGCCTTGGAGTCCTAGACCTCTGGAAGCCACAGCTGAGTCTAAGTACGGCCCTCCTTGTCC |
| | | TCCATGTCCTGCTCCAGAAGCTGCTGGCGGCCCTTCCGTGTTTCTGTTCCCTCCAAAGCCTA |
| | | AGGACACCCTGATGATCTCTCGGACCCCTGAAGTGACCTGCGTGGTGGTGGATGTGTCCCA |
| | | AGAGGATCCCGAGGTGCAGTTCAATTGGTACGTGGACGGCGTGGAAGTGCACAACGCCAA |
| | | GACCAAGCCTAGAGAGGAACAGTTCAACTCCACCTACAGAGTGGTGTCCGTGCTGACCGT |
| | | GCTGCACCAGGATTGGCTGAACGGCAAAGAGTACAAGTGCAAGGTGTCCAACAAGGGCCT |
| | | GCCTTCCAGCATCGAAAAGACCATCTCCAAGGCCAAGGGCCAGCCTAGGGAACCCCAGGT |
| | | TTACACCCTGCCTCCAAGCCAAGAGGAAATGACCAAGAACCAGGTGTCCCTGACCTGCCTG |
| | | GTCAAGGGCTTCTACCCTTCCGATATCGCCGTGGAATGGGAGAGCAATGGCCAGCCTGAGA |
| | | ACAACTACAAGACCACACCTCCTGTGCTGGACTCCGACGGCTCCTTCTTTCTGTACTCCCGC |
| | | CTGACCGTGGACAAGTCCAGATGGCAAGAGGGCAACGTGTTCTCCTGCTCCGTGATGCACG |
| | | AGGCCCTGCACAATCACTACACCCAGAAGTCCCTGTCTCTGTCCCTGGGCAAA |
| 39 | FLT3L (A C-term | ACCCAGGACTGCTCCTTCCAGCACTCCCCTATCTCTTCCGACTTCGCCGTGAAGATCAGAG |
| | 5aa (PTAPQ; SEQ | AGCTGTCCGACTACCTGCTGCAGGACTATCCTGTGACCGTGGCCAGCAACCTGCAGGATGA |
| | ID NO: 85))-hG4 | AGAACTGTGTGGTGGACTGTGGCGACTGGTGCTGGCTCAGAGATGGATGGAAAGACTGAA |
| | S228P/ | AACCGTGGCCGGCTCCAAGATGCAGGGACTGCTGGAAAGAGTGAACACAGAGATCCACTT |
| | F234A/ | CGTGACCAAGTGCGCCTTTCAGCCTCCTCCATCCTGCCTGAGATTCGTGCAGACCAACATCT |
| | L235A | CCCGGCTGCTGCAAGAGACATCTGAGCAGCTGGTGGCCCTGAAGCCTTGGATCACCCGGCA |
| | | GAACTTCTCTCGGTGCCTGGAACTGCAGTGTCAGCCTGATTCTTCTACCCTGCCTCCACCTT |
| | | GGAGCCCTAGACCTCTGGAAGCTACCGCCGAGTCTAAGTACGGACCTCCTTGTCCTCCATG |
| | | TCCTGCTCCAGAAGCTGCTGGCGGACCAAGCGTTTTCCTGTTTCCTCCAAAGCCTAAGGAC |
| | | ACCCTGATGATCTCTCGGACCCCTGAAGTGACCTGCGTGGTGGTGGATGTGTCTCAAGAGG |
| | | ACCCCGAGGTGCAGTTCAATTGGTACGTGGACGGCGTGGAAGTGCACAACGCCAAGACCA |
| | | AGCCTAGAGAGGAACAGTTCAACTCCACCTACAGAGTGGTGTCCGTGCTGACCGTGCTGCA |
| | | CCAGGATTGGCTGAACGGCAAAGAGTACAAGTGCAAGGTGTCCAACAAGGGCCTGCCTAG |
| | | CTCCATCGAAAAGACCATCTCCAAGGCCAAGGGCCAGCCAAGAGAACCTCAGGTGTACAC |
| | | ACTGCCTCCAAGCCAAGAGGAAATGACCAAGAACCAGGTGTCCCTGACCTGCCTGGTCAA |
| | | GGGCTTCTACCCATCCGATATCGCCGTGGAATGGGAGTCTAACGGCCAGCCTGAGAACAAC |
| | | TACAAGACCACACCTCCTGTGCTGGACTCCGACGGCTCCTTCTTTCTGTACTCTCGCCTGAC |
| | | CGTGGACAAGTCTAGATGGCAAGAGGGCAACGTGTTCTCCTGCTCTGTGATGCACGAGGCC |
| | | CTGCACAACCACTACACCCAGAAGTCCCTGTCTCTGTCCCTGGGCAAA |
| 40 | FLT3L ECD | ACACAGGATTGCAGCTTCCAGCACAGCCCCATCAGCAGCGATTTCGCCGTGAAGATCAGA |
| | (Δ C-term 10aa | GAGCTGAGCGACTACCTGCTGCAGGACTACCCTGTGACCGTGGCCAGCAATCTGCAGGAC |
| | (LEATAPTAPQ; | GAAGAACTGTGTGGTGGACTGTGGCGACTGGTGCTGGCTCAGAGATGGATGGAACGGCTG |
| | SEQ ID NO: 90))- | AAAACAGTGGCCGGCAGCAAGATGCAGGGACTGCTGGAAAGAGTGAACACCGAGATCCA |
| | hingeless hG1 | CTTCGTGACCAAGTGCGCCTTCCAGCCTCCTCCATCCTGCCTGAGATTCGTGCAGACCAAC |
| | | ATCAGCAGACTGCTGCAAGAGACAAGCGAGCAGCTGGTGGCCCTGAAGCCTTGGATCACC |
| | | AGACAGAACTTCAGCCGGTGCCTGGAACTGCAGTGTCAGCCCGATAGCAGCACACTGCCTC |
| | | CGCCTTGGAGTCCTAGACCTGGGGGACCGTCAGTCTTCCTCTTCCCCCCAAAACCCAAGGA |
| | | CACCCTCATGATCTCCCGGACCCCTGAGGTCACATGCGTGGTGGTGGACGTGAGCCACGAA |
| | | GACCCTGAGGTCAAGTTCAACTGGTACGTGGACGGCGTGGAGGTGCATAATGCCAAGACA |
| | | AAGCCGCGGGAGGAGCAGTACAACAGCACGTACCGTGTGGTCAGCGTCCTCACCGTCCTG |
| | | CACCAGGACTGGCTGAATGGCAAGGAGTACAAGTGCAAGGTCTCCAACAAAGCCCTCCCA |
| | | GCCCCCATCGAGAAAACCATCTCCAAAGCCAAAGGGCAGCCCCGAGAACCACAGGTGTAC |
| | | ACCCTGCCCCCATCCCGGGAGGAGATGACCAAGAACCAGGTCAGCCTGACCTGCCTGGTC |
| | | AAAGGCTTCTATCCCAGCGACATCGCCGTGGAGTGGGAGAGCAATGGGCAGCCGGAGAAC |
| | | AACTACAAGACCACGCCTCCCGTGCTGGACTCCGACGGCTCCTTCTTCCTCTACAGCAAGC |
| | | TCACCGTGGACAAGAGCAGGTGGCAGCAGGGGAACGTCTTCTCATGCTCCGTGATGCATG |
| | | AGGCTCTGCACAACCACTACACGCAGAAGAGCCTCTCCCTGTCTCCGGGTAAA |
| 41 | FLT3L ECD | ACCCAGGACTGCTCCTTCCAGCACTCCCCTATCTCTTCCGACTTCGCCGTGAAGATCAGAG |
| | (Δ C-term 10aa | AGCTGTCCGACTACCTGCTGCAGGACTATCCTGTGACCGTGGCCAGCAACCTGCAGGATGA |
| | (LEATAPTAPQ; | AGAACTGTGTGGTGGACTGTGGCGACTGGTGCTGGCTCAGAGATGGATGGAAAGACTGAA |
| | SEQ ID NO: 90))- | AACCGTGGCCGGCTCCAAGATGCAGGGACTGCTGGAAAGAGTGAACACAGAGATCCACTT |
| | hingeless hG1 | CGTGACCAAGTGCGCCTTTCAGCCTCCTCCATCCTGCCTGAGATTCGTGCAGACCAACATCT |
| | | CCCGGCTGCTGCAAGAGACATCTGAGCAGCTGGTGGCCCTGAAGCCTTGGATCACCCGGCA |
| | | GAACTTCTCTCGGTGCCTGGAACTGCAGTGTCAGCCTGATTCTTCTACCCTGCCTCCACCTT |
| | | GGAGTCCTAGACCTGGCGGACCAAGCGTGTTCCTGTTTCCTCCAAAGCCTAAGGACACCCT |
| | | GATGATCTCTCGGACCCCTGAAGTGACCTGCGTGGTGGTGGATGTGTCTCACGAGGATCCC |
| | | GAAGTGAAGTTCAATTGGTACGTGGACGGCGTGGAAGTGCACAACGCCAAGACCAAGCCT |
| | | AGAGAGGAACAGTACAACTCCACCTACAGAGTGGTGTCCGTGCTGACCGTGCTGCACCAG |
| | | GATTGGCTGAACGGCAAAGAGTACAAGTGCAAGGTGTCCAACAAGGCCCTGCCTGCTCCT |
| | | ATCGAAAAGACCATCTCCAAGGCCAAGGGCCAGCCTAGGGAACCTCAGGTTTACACCCTG |
| | | CCACCTAGCCGGGAAGAGATGACCAAAAACCAGGTGTCCCTGACCTGCCTGGTCAAGGGC |
| | | TTCTACCCATCCGATATCGCCGTGGAATGGGAGTCTAACGGCCAGCCTGAGAACAACTACA |
| | | AGACCACACCTCCTGTGCTGGACTCCGACGGCTCATTCTTCCTGTACTCCAAGCTGACAGT |
| | | GGACAAGTCTCGGTGGCAGCAGGGCAACGTGTTCTCCTGTTCTGTGATGCACGAGGCCCTG |
| | | CACAACCACTACACCCAGAAGTCCCTGTCTCTGTCCCCTGGCAAA |
| 42 | FLT3L ECD | ACACAGGATTGCAGCTTCCAGCACAGCCCCATCAGCAGCGATTTCGCCGTGAAGATCAGA |
| | (Δ C-term 10aa | GAGCTGAGCGACTACCTGCTGCAGGACTACCCTGTGACCGTGGCCAGCAATCTGCAGGAC |
| | (LEATAPTAPQ; | GAAGAACTGTGTGGTGGACTGTGGCGACTGGTGCTGGCTCAGAGATGGATGGAACGGCTG |
| | SEQ ID NO: 90))- | AAAACAGTGGCCGGCAGCAAGATGCAGGGACTGCTGGAAAGAGTGAACACCGAGATCCA |
| | hG4 S228P/ | CTTCGTGACCAAGTGCGCCTTCCAGCCTCCTCCATCCTGCCTGAGATTCGTGCAGACCAAC |
| | F234A/ | ATCAGCAGACTGCTGCAAGAGACAAGCGAGCAGCTGGTGGCCCTGAAGCCTTGGATCACC |
| | L235A | AGACAGAACTTCAGCCGGTGCCTGGAACTGCAGTGTCAGCCCGATAGCAGCACACTGCCTC |
| | | CGCCTTGGAGTCCTAGACCTGAGTCTAAGTACGGCCCTCCTTGTCCTCCATGTCCTGCTCCA |
| | | GAAGCTGCTGGCGGCCCTTCCGTGTTTCTGTTCCCTCCAAAGCCTAAGGACACCCTGATGA |
| | | TCTCTCGGACCCCTGAAGTGACCTGCGTGGTGGTGGATGTGTCCCAAGAGGATCCCGAGGT |
| | | GCAGTTCAATTGGTACGTGGACGGCGTGGAAGTGCACAACGCCAAGACCAAGCCTAGAGA |
| | | GGAACAGTTCAACTCCACCTACAGAGTGGTGTCCGTGCTGACCGTGCTGCACCAGGATTGG |
| | | CTGAACGGCAAAGAGTACAAGTGCAAGGTGTCCAACAAGGGCCTGCCTTCCAGCATCGAA |
| | | AAGACCATCTCCAAGGCCAAGGGCCAGCCTAGGGAACCCCAGGTTTACACCCTGCCTCCA |
| | | AGCCAAGAGGAAATGACCAAGAACCAGGTGTCCCTGACCTGCCTGGTCAAGGGCTTCTAC |
| | | CCTTCCGATATCGCCGTGGAATGGGAGAGCAATGGCCAGCCTGAGAACAACTACAAGACC |
| | | ACACCTCCTGTGCTGGACTCCGACGGCTCCTTCTTTCTGTACTCCCGCCTGACCGTGGACAA |
| | | GTCCAGATGGCAAGAGGGCAACGTGTTCTCCTGCTCCGTGATGCACGAGGCCCTGCACAAT |
| | | CACTACACCCAGAAGTCCCTGTCTCTGTCCCTGGGCAAA |
| 43 | FLT3L ECD (Δ C- | ACCCAGGACTGCTCCTTCCAGCACTCCCCTATCTCTTCCGACTTCGCCGTGAAGATCAGAG |
| | term 10aa | AGCTGTCCGACTACCTGCTGCAGGACTATCCTGTGACCGTGGCCAGCAACCTGCAGGATGA |
| | (LEATAPTAPQ; | AGAACTGTGTGGTGGACTGTGGCGACTGGTGCTGGCTCAGAGATGGATGGAAAGACTGAA |
| | SEQ ID NO: 90))- | AACCGTGGCCGGCTCCAAGATGCAGGGACTGCTGGAAAGAGTGAACACAGAGATCCACTT |
| | hG4 S228P/ | CGTGACCAAGTGCGCCTTTCAGCCTCCTCCATCCTGCCTGAGATTCGTGCAGACCAACATCT |
| | F234A/ | CCCGGCTGCTGCAAGAGACATCTGAGCAGCTGGTGGCCCTGAAGCCTTGGATCACCCGGCA |
| | L235A | GAACTTCTCTCGGTGCCTGGAACTGCAGTGTCAGCCTGATTCTTCTACCCTGCCTCCACCTT |
| | | GGAGCCCTCGGCCTGAATCTAAGTATGGCCCTCCTTGTCCTCCATGTCCTGCTCCAGAAGCT |
| | | GCTGGCGGACCAAGCGTTTTCCTGTTTCCTCCAAAGCCTAAGGACACCCTGATGATCTCTC |
| | | GGACCCCTGAAGTGACCTGCGTGGTGGTGGATGTGTCTCAAGAGGACCCCGAGGTGCAGTT |
| | | CAATTGGTACGTGGACGGCGTGGAAGTGCACAACGCCAAGACCAAGCCTAGAGAGGAACA |
| | | GTTCAACTCCACCTACAGAGTGGTGTCCGTGCTGACCGTGCTGCACCAGGATTGGCTGAAC |
| | | GGCAAAGAGTACAAGTGCAAGGTGTCCAACAAGGGCCTGCCTAGCTCCATCGAAAAGACC |
| | | ATCTCCAAGGCCAAGGGCCAGCCAAGAGAACCTCAGGTGTACACACTGCCTCCAAGCCAA |
| | | GAGGAAATGACCAAGAACCAGGTGTCCCTGACCTGCCTGGTCAAGGGCTTCTACCCATCCG |
| | | ATATCGCCGTGGAATGGGAGTCTAACGGCCAGCCTGAGAACAACTACAAGACCACACCTC |
| | | CTGTGCTGGACTCCGACGGCTCCTTCTTTCTGTACTCTCGCCTGACCGTGGACAAGTCTAGA |
| | | TGGCAAGAGGGCAACGTGTTCTCCTGCTCTGTGATGCACGAGGCCCTGCACAACCACTACA |
| | | CCCAGAAGTCCCTGTCTCTGTCCCTGGGCAAA |
| 44 | FLT3L ECD- | ACACAGGATTGCAGCTTCCAGCACAGCCCCATCAGCAGCGATTTCGCCGTGAAGATCAGA |
| | hingeless hG1 | GAGCTGAGCGACTACCTGCTGCAGGACTACCCTGTGACCGTGGCCAGCAATCTGCAGGAC |
| | (M252Y/S254T/ | GAAGAACTGTGTGGTGGACTGTGGCGACTGGTGCTGGCTCAGAGATGGATGGAACGGCTG |
| | T256E) | AAAACAGTGGCCGGCAGCAAGATGCAGGGACTGCTGGAAAGAGTGAACACCGAGATCCA |
| | | CTTCGTGACCAAGTGCGCCTTCCAGCCTCCTCCATCCTGCCTGAGATTCGTGCAGACCAAC |
| | | ATCAGCAGACTGCTGCAAGAGACAAGCGAGCAGCTGGTGGCCCTGAAGCCTTGGATCACC |
| | | AGACAGAACTTCAGCCGGTGCCTGGAACTGCAGTGTCAGCCCGATAGCAGCACACTGCCTC |
| | | CGCCTTGGAGTCCTAGACCTCTGGAAGCCACAGCTCCCACCGCTCCTCAAGGCGGACCGTC |
| | | AGTCTTCCTCTTCCCCCCAAAACCCAAGGACACCCTCTACATCACCCGGGAACCTGAGGTC |
| | | ACATGCGTGGTGGTGGACGTGAGCCACGAAGACCCTGAGGTCAAGTTCAACTGGTACGTG |
| | | GACGGCGTGGAGGTGCATAATGCCAAGACAAAGCCGCGGGAGGAGCAGTACAACAGCAC |
| | | GTACCGTGTGGTCAGCGTCCTCACCGTCCTGCACCAGGACTGGCTGAATGGCAAGGAGTAC |
| | | AAGTGCAAGGTCTCCAACAAAGCCCTCCCAGCCCCCATCGAGAAAACCATCTCCAAAGCC |
| | | AAAGGGCAGCCCCGAGAACCACAGGTGTACACCCTGCCCCCATCCCGGGAGGAGATGACC |
| | | AAGAACCAGGTCAGCCTGACCTGCCTGGTCAAAGGCTTCTATCCCAGCGACATCGCCGTGG |
| | | AGTGGGAGAGCAATGGGCAGCCGGAGAACAACTACAAGACCACGCCTCCCGTGCTGGACT |
| | | CCGACGGCTCCTTCTTCCTCTACAGCAAGCTCACCGTGGACAAGAGCAGGTGGCAGCAGGG |
| | | GAACGTCTTCTCATGCTCCGTGATGCATGAGGCTCTGCACAACCACTACACGCAGAAGAGC |
| | | CTCTCCCTGTCTCCGGGTAAA |
| 45 | FLT3L ECD- | ACCCAGGACTGCTCCTTCCAGCACTCCCCTATCTCTTCCGACTTCGCCGTGAAGATCAGAG |
| | hingeless hG1 | AGCTGTCCGACTACCTGCTGCAGGACTATCCTGTGACCGTGGCCAGCAACCTGCAGGATGA |
| | (M252Y/S254T/ | AGAACTGTGTGGTGGACTGTGGCGACTGGTGCTGGCTCAGAGATGGATGGAAAGACTGAA |
| | T256E) | AACCGTGGCCGGCTCCAAGATGCAGGGACTGCTGGAAAGAGTGAACACAGAGATCCACTT |
| | | CGTGACCAAGTGCGCCTTTCAGCCTCCTCCATCCTGCCTGAGATTCGTGCAGACCAACATCT |
| | | CCCGGCTGCTGCAAGAGACATCTGAGCAGCTGGTGGCCCTGAAGCCTTGGATCACCCGGCA |
| | | GAACTTCTCTCGGTGCCTGGAACTGCAGTGTCAGCCTGATTCTTCTACCCTGCCTCCACCTT |
| | | GGAGCCCTAGACCTTTGGAGGCTACAGCTCCTACTGCTCCTCAAGGCGGCCCAAGCGTTTT |
| | | CCTGTTTCCTCCAAAGCCTAAGGACACCCTGTACATCACCCGCGAGCCTGAAGTGACATGC |
| | | GTGGTGGTGGATGTGTCCCACGAGGACCCCGAAGTGAAGTTCAATTGGTACGTGGACGGC |
| | | GTGGAAGTGCACAACGCCAAGACCAAGCCTAGAGAGGAACAGTACAACTCCACCTACAGA |
| | | GTGGTGTCCGTGCTGACCGTGCTGCACCAGGATTGGCTGAACGGCAAAGAGTACAAGTGC |
| | | AAGGTGTCCAACAAGGCCCTGCCTGCTCCTATCGAAAAGACCATCTCCAAGGCCAAGGGC |
| | | CAGCCTAGGGAACCTCAGGTTTACACCCTGCCACCTAGCCGGGAAGAGATGACCAAAAAC |
| | | CAGGTGTCCCTGACCTGCCTGGTCAAGGGCTTCTACCCATCCGATATCGCCGTGGAATGGG |
| | | AGTCTAACGGCCAGCCTGAGAACAACTACAAGACCACACCTCCTGTGCTGGACTCCGACG |
| | | GCTCATTCTTCCTGTACTCCAAGCTGACAGTGGACAAGTCTCGGTGGCAGCAGGGCAACGT |
| | | GTTCTCCTGTTCTGTGATGCACGAGGCCCTGCACAACCACTACACCCAGAAGTCCCTGTCTC |
| | | TGTCCCCTGGCAAA |
| 46 | FLT3L ECD | ACACAGGATTGCAGCTTCCAGCACAGCCCCATCAGCAGCGATTTCGCCGTGAAGATCAGA |
| | (Δ C-term 5aa | GAGCTGAGCGACTACCTGCTGCAGGACTACCCTGTGACCGTGGCCAGCAATCTGCAGGAC |
| | (PTAPQ; | GAAGAACTGTGTGGTGGACTGTGGCGACTGGTGCTGGCTCAGAGATGGATGGAACGGCTG |
| | SEQ ID NO: 85)) - | AAAACAGTGGCCGGCAGCAAGATGCAGGGACTGCTGGAAAGAGTGAACACCGAGATCCA |
| | hingeless hG1 | CTTCGTGACCAAGTGCGCCTTCCAGCCTCCTCCATCCTGCCTGAGATTCGTGCAGACCAAC |
| | (M252Y/S254T/ | ATCAGCAGACTGCTGCAAGAGACAAGCGAGCAGCTGGTGGCCCTGAAGCCTTGGATCACC |
| | T256E) | AGACAGAACTTCAGCCGGTGCCTGGAACTGCAGTGTCAGCCCGATAGCAGCACACTGCCTC |
| | | CGCCTTGGAGTCCTAGACCTCTGGAAGCCACAGCTGGGGGACCGTCAGTCTTCCTCTTCCC |
| | | CCCAAAACCCAAGGACACCCTCTACATCACCCGGGAACCTGAGGTCACATGCGTGGTGGT |
| | | GGACGTGAGCCACGAAGACCCTGAGGTCAAGTTCAACTGGTACGTGGACGGCGTGGAGGT |
| | | GCATAATGCCAAGACAAAGCCGCGGGAGGAGCAGTACAACAGCACGTACCGTGTGGTCAG |
| | | CGTCCTCACCGTCCTGCACCAGGACTGGCTGAATGGCAAGGAGTACAAGTGCAAGGTCTCC |
| | | AACAAAGCCCTCCCAGCCCCCATCGAGAAAACCATCTCCAAAGCCAAAGGGCAGCCCCGA |
| | | GAACCACAGGTGTACACCCTGCCCCCATCCCGGGAGGAGATGACCAAGAACCAGGTCAGC |
| | | CTGACCTGCCTGGTCAAAGGCTTCTATCCCAGCGACATCGCCGTGGAGTGGGAGAGCAATG |
| | | GGCAGCCGGAGAACAACTACAAGACCACGCCTCCCGTGCTGGACTCCGACGGCTCCTTCTT |
| | | CCTCTACAGCAAGCTCACCGTGGACAAGAGCAGGTGGCAGCAGGGGAACGTCTTCTCATG |
| | | CTCCGTGATGCATGAGGCTCTGCACAACCACTACACGCAGAAGAGCCTCTCCCTGTCTCCG |
| | | GGTAAA |
| 47 | FLT3L ECD | ACCCAGGACTGCTCCTTCCAGCACTCCCCTATCTCTTCCGACTTCGCCGTGAAGATCAGAG |
| | (Δ C-term 5aa | AGCTGTCCGACTACCTGCTGCAGGACTATCCTGTGACCGTGGCCAGCAACCTGCAGGATGA |
| | (PTAPQ; | AGAACTGTGTGGTGGACTGTGGCGACTGGTGCTGGCTCAGAGATGGATGGAAAGACTGAA |
| | SEQ ID NO: 85)) - | AACCGTGGCCGGCTCCAAGATGCAGGGACTGCTGGAAAGAGTGAACACAGAGATCCACTT |
| | hingeless hG1 | CGTGACCAAGTGCGCCTTTCAGCCTCCTCCATCCTGCCTGAGATTCGTGCAGACCAACATCT |
| | (M252Y/S254T/ | CCCGGCTGCTGCAAGAGACATCTGAGCAGCTGGTGGCCCTGAAGCCTTGGATCACCCGGCA |
| | T256E) | GAACTTCTCTCGGTGCCTGGAACTGCAGTGTCAGCCTGATTCTTCTACCCTGCCTCCACCTT |
| | | GGAGCCCTAGACCTTTGGAAGCTACAGCTGGCGGCCCAAGCGTGTTCCTGTTTCCTCCAAA |
| | | GCCTAAGGACACCCTGTACATCACCCGCGAGCCTGAAGTGACATGCGTGGTGGTGGATGTG |
| | | TCCCACGAGGACCCCGAAGTGAAGTTCAATTGGTACGTGGACGGCGTGGAAGTGCACAAC |
| | | GCCAAGACCAAGCCTAGAGAGGAACAGTACAACTCCACCTACAGAGTGGTGTCCGTGCTG |
| | | ACCGTGCTGCACCAGGATTGGCTGAACGGCAAAGAGTACAAGTGCAAGGTGTCCAACAAG |
| | | GCCCTGCCTGCTCCTATCGAAAAGACCATCTCCAAGGCCAAGGGCCAGCCTAGGGAACCTC |
| | | AGGTTTACACCCTGCCACCTAGCCGGGAAGAGATGACCAAAAACCAGGTGTCCCTGACCT |
| | | GCCTGGTCAAGGGCTTCTACCCATCCGATATCGCCGTGGAATGGGAGTCTAACGGCCAGCC |
| | | TGAGAACAACTACAAGACCACACCTCCTGTGCTGGACTCCGACGGCTCATTCTTCCTGTAC |
| | | TCCAAGCTGACAGTGGACAAGTCTCGGTGGCAGCAGGGCAACGTGTTCTCCTGTTCTGTGA |
| | | TGCACGAGGCCCTGCACAACCACTACACCCAGAAGTCCCTGTCTCTGTCCCCTGGCAAA |
| 48 | FLT3L ECD - hG4 | ACACAGGATTGCAGCTTCCAGCACAGCCCCATCAGCAGCGATTTCGCCGTGAAGATCAGA |
| | S228P/L235E/ | GAGCTGAGCGACTACCTGCTGCAGGACTACCCTGTGACCGTGGCCAGCAATCTGCAGGAC |
| | M252Y/ | GAAGAACTGTGTGGTGGACTGTGGCGACTGGTGCTGGCTCAGAGATGGATGGAACGGCTG |
| | S254T/T256E) | AAAACAGTGGCCGGCAGCAAGATGCAGGGACTGCTGGAAAGAGTGAACACCGAGATCCA |
| | | CTTCGTGACCAAGTGCGCCTTCCAGCCTCCTCCATCCTGCCTGAGATTCGTGCAGACCAAC |
| | | ATCAGCAGACTGCTGCAAGAGACAAGCGAGCAGCTGGTGGCCCTGAAGCCTTGGATCACC |
| | | AGACAGAACTTCAGCCGGTGCCTGGAACTGCAGTGTCAGCCCGATAGCAGCACACTGCCTC |
| | | CGCCTTGGAGTCCTAGACCTCTGGAAGCCACAGCTCCCACCGCTCCTCAAGAATCTAAGTA |
| | | CGGCCCTCCCTGCCCTCCTTGCCCAGCCCCTGAATTTGAGGGCGGACCCTCCGTGTTCCTGT |
| | | TCCCCCCAAAGCCCAAGGACACCCTGTACATCACCCGGGAACCCGAAGTGACCTGCGTGGT |
| | | GGTGGATGTGTCCCAGGAAGATCCCGAGGTGCAGTTCAATTGGTACGTGGACGGCGTGGA |
| | | AGTGCACAACGCCAAGACCAAGCCCAGAGAGGAACAGTTCAACAGCACCTACCGGGTGGT |
| | | GTCCGTGCTGACAGTGCTGCACCAGGACTGGCTGAACGGCAAAGAGTACAAGTGCAAGGT |
| | | GTCCAACAAGGGCCTGCCCAGCTCCATCGAGAAAACCATCAGCAAGGCCAAGGGCCAGCC |
| | | CCGCGAACCCCAGGTGTACACACTGCCTCCAAGCCAGGAAGAGATGACCAAGAACCAGGT |
| | | GTCCCTGACCTGTCTCGTGAAAGGCTTCTACCCCTCCGATATCGCCGTGGAATGGGAGAGC |
| | | AACGGCCAGCCCGAGAACAACTACAAGACCACCCCCCCTGTGCTGGACAGCGACGGCTCA |
| | | TTCTTCCTGTACAGCAGACTGACCGTGGACAAGAGCCGGTGGCAGGAAGGCAACGTGTTC |
| | | AGCTGCAGCGTGATGCACGAGGCCCTGCACAACCACTACACCCAGAAGTCCCTGTCTCTGT |
| | | CCCTGGGCAAA |
| 49 | FLT3L ECD - hG4 | ACCCAGGACTGCTCCTTCCAGCACTCCCCTATCTCTTCCGACTTCGCCGTGAAGATCAGAG |
| | S228P/L235E/ | AGCTGTCCGACTACCTGCTGCAGGACTATCCTGTGACCGTGGCCAGCAACCTGCAGGATGA |
| | M252Y/ | AGAACTGTGTGGTGGACTGTGGCGACTGGTGCTGGCTCAGAGATGGATGGAAAGACTGAA |
| | S254T/T256E) | AACCGTGGCCGGCTCCAAGATGCAGGGACTGCTGGAAAGAGTGAACACAGAGATCCACTT |
| | | CGTGACCAAGTGCGCCTTTCAGCCTCCTCCATCCTGCCTGAGATTCGTGCAGACCAACATCT |
| | | CCCGGCTGCTGCAAGAGACATCTGAGCAGCTGGTGGCCCTGAAGCCTTGGATCACCCGGCA |
| | | GAACTTCTCTCGGTGCCTGGAACTGCAGTGTCAGCCTGATTCTTCTACCCTGCCTCCACCTT |
| | | GGAGCCCTAGACCTTTGGAGGCTACAGCTCCTACCGCTCCTCAAGAGTCTAAGTACGGCCC |
| | | TCCTTGTCCTCCATGTCCTGCTCCAGAATTTGAAGGCGGCCCAAGCGTGTTCCTGTTTCCTC |
| | | CAAAGCCTAAGGACACCCTGTACATCACCCGCGAGCCTGAAGTGACATGCGTGGTGGTGG |
| | | ATGTGTCCCAAGAGGACCCCGAGGTGCAGTTCAATTGGTACGTGGACGGCGTGGAAGTGC |
| | | ACAACGCCAAGACCAAGCCTAGAGAGGAACAGTTCAACTCCACCTACAGAGTGGTGTCCG |
| | | TGCTGACCGTGCTGCACCAGGATTGGCTGAACGGCAAAGAGTACAAGTGCAAGGTGTCCA |
| | | ACAAGGGCCTGCCTAGCTCCATCGAAAAGACCATCTCCAAGGCCAAGGGCCAGCCAAGAG |
| | | AACCTCAGGTGTACACACTGCCTCCAAGCCAAGAGGAAATGACCAAGAACCAGGTGTCCC |
| | | TGACCTGCCTGGTCAAGGGCTTCTACCCATCCGATATCGCCGTGGAATGGGAGTCTAACGG |
| | | CCAGCCTGAGAACAACTACAAGACCACACCTCCTGTGCTGGACTCCGACGGCTCCTTCTTT |
| | | CTGTACTCTCGCCTGACCGTGGACAAGTCTAGATGGCAAGAGGGCAACGTGTTCTCCTGCT |
| | | CTGTGATGCACGAGGCCCTGCACAACCACTACACCCAGAAGTCCCTGTCTCTGTCCCTGGG |
| | | CAAA |
| 50 | FLT3L ECD - hG4 | ACACAGGATTGCAGCTTCCAGCACAGCCCCATCAGCAGCGATTTCGCCGTGAAGATCAGA |
| | (S228P/F234A/ | GAGCTGAGCGACTACCTGCTGCAGGACTACCCTGTGACCGTGGCCAGCAATCTGCAGGAC |
| | L235A/M252Y/ | GAAGAACTGTGTGGTGGACTGTGGCGACTGGTGCTGGCTCAGAGATGGATGGAACGGCTG |
| | S254T/T256E) | AAAACAGTGGCCGGCAGCAAGATGCAGGGACTGCTGGAAAGAGTGAACACCGAGATCCA |
| | | CTTCGTGACCAAGTGCGCCTTCCAGCCTCCTCCATCCTGCCTGAGATTCGTGCAGACCAAC |
| | | ATCAGCAGACTGCTGCAAGAGACAAGCGAGCAGCTGGTGGCCCTGAAGCCTTGGATCACC |
| | | AGACAGAACTTCAGCCGGTGCCTGGAACTGCAGTGTCAGCCCGATAGCAGCACACTGCCTC |
| | | CGCCTTGGAGTCCTAGACCTCTGGAAGCCACAGCTCCCACCGCTCCTCAAGAGTCTAAGTA |
| | | CGGCCCTCCTTGTCCTCCATGTCCTGCTCCAGAAGCTGCTGGCGGCCCTTCCGTGTTTCTGT |
| | | TCCCTCCAAAGCCTAAGGACACCCTGTACATCACCCGGGAACCCGAAGTGACCTGCGTGGT |
| | | GGTGGATGTGTCCCAGGAAGATCCCGAGGTGCAGTTCAATTGGTACGTGGACGGCGTGGA |
| | | AGTGCACAACGCCAAGACCAAGCCCAGAGAGGAACAGTTCAACAGCACCTACCGGGTGGT |
| | | GTCCGTGCTGACAGTGCTGCACCAGGACTGGCTGAACGGCAAAGAGTACAAGTGCAAGGT |
| | | GTCCAACAAGGGCCTGCCCAGCTCCATCGAGAAAACCATCAGCAAGGCCAAGGGCCAGCC |
| | | CCGCGAACCCCAGGTGTACACACTGCCTCCAAGCCAGGAAGAGATGACCAAGAACCAGGT |
| | | GTCCCTGACCTGTCTCGTGAAAGGCTTCTACCCCTCCGATATCGCCGTGGAATGGGAGAGC |
| | | AACGGCCAGCCCGAGAACAACTACAAGACCACCCCCCCTGTGCTGGACAGCGACGGCTCA |
| | | TTCTTCCTGTACAGCAGACTGACCGTGGACAAGAGCCGGTGGCAGGAAGGCAACGTGTTC |
| | | AGCTGCAGCGTGATGCACGAGGCCCTGCACAACCACTACACCCAGAAGTCCCTGTCTCTGT |
| | | CCCTGGGCAAA |
| 51 | FLT3L ECD - hG4 | ACCCAGGACTGCTCCTTCCAGCACTCCCCTATCTCTTCCGACTTCGCCGTGAAGATCAGAG |
| | (S228P/F234A/ | AGCTGTCCGACTACCTGCTGCAGGACTATCCTGTGACCGTGGCCAGCAACCTGCAGGATGA |
| | L235A/M252Y/ | AGAACTGTGTGGTGGACTGTGGCGACTGGTGCTGGCTCAGAGATGGATGGAAAGACTGAA |
| | S254T/T256E) | AACCGTGGCCGGCTCCAAGATGCAGGGACTGCTGGAAAGAGTGAACACAGAGATCCACTT |
| | | CGTGACCAAGTGCGCCTTTCAGCCTCCTCCATCCTGCCTGAGATTCGTGCAGACCAACATCT |
| | | CCCGGCTGCTGCAAGAGACATCTGAGCAGCTGGTGGCCCTGAAGCCTTGGATCACCCGGCA |
| | | GAACTTCTCTCGGTGCCTGGAACTGCAGTGTCAGCCTGATTCTTCTACCCTGCCTCCACCTT |
| | | GGAGCCCTAGACCTTTGGAGGCTACAGCTCCTACCGCTCCTCAAGAGTCTAAGTACGGCCC |
| | | TCCTTGTCCTCCATGTCCTGCTCCAGAAGCTGCTGGCGGACCAAGCGTTTTCCTGTTTCCTC |
| | | CAAAGCCTAAGGACACCCTGTACATCACCCGCGAGCCTGAAGTGACATGCGTGGTGGTGG |
| | | ATGTGTCCCAAGAGGACCCCGAGGTGCAGTTCAATTGGTACGTGGACGGCGTGGAAGTGC |
| | | ACAACGCCAAGACCAAGCCTAGAGAGGAACAGTTCAACTCCACCTACAGAGTGGTGTCCG |
| | | TGCTGACCGTGCTGCACCAGGATTGGCTGAACGGCAAAGAGTACAAGTGCAAGGTGTCCA |
| | | ACAAGGGCCTGCCTAGCTCCATCGAAAAGACCATCTCCAAGGCCAAGGGCCAGCCAAGAG |
| | | AACCTCAGGTGTACACACTGCCTCCAAGCCAAGAGGAAATGACCAAGAACCAGGTGTCCC |
| | | TGACCTGCCTGGTCAAGGGCTTCTACCCATCCGATATCGCCGTGGAATGGGAGTCTAACGG |
| | | CCAGCCTGAGAACAACTACAAGACCACACCTCCTGTGCTGGACTCCGACGGCTCCTTCTTT |
| | | CTGTACTCTCGCCTGACCGTGGACAAGTCTAGATGGCAAGAGGGCAACGTGTTCTCCTGCT |
| | | CTGTGATGCACGAGGCCCTGCACAACCACTACACCCAGAAGTCCCTGTCTCTGTCCCTGGG |
| | | CAAA |
| 52 | Aglyco-FLT3L | ATGACAGTTTTGGCTCCAGCTTGGTCCCCTACAACCTACCTGCTGCTGCTGTTGCTGCTCTC |
| | ECD | CTCTGGCCTGTCTGGCACCCAGGACTGTTCCTTCCAGCACTCCCCTATCTCCAGCGACTTCG |
| | (S128A/S151A) | CCGTGAAGATCAGAGAGCTGTCCGACTATCTGCTGCAGGACTACCCTGTGACCGTGGCCAG |
| | hingeless hG1 | CAATCTGCAGGACGAAGAACTGTGTGGTGGCCTGTGGCGACTGGTGTTGGCTCAGAGATG |
| | (M252Y/S254T/ | GATGGAACGGCTGAAAACCGTGGCCGGCTCTAAGATGCAGGGCCTGCTGGAAAGAGTGAA |
| | T256E) | CACCGAGATCCACTTCGTGACCAAGTGCGCCTTTCAGCCTCCTCCATCCTGCCTGAGATTCG |
| | | TGCAGACCAATATCGCCCGGCTGCTGCAAGAGACATCCGAGCAGCTGGTGGCTCTGAAGC |
| | | CCTGGATCACCAGACAGAACTTCGCCCGGTGTCTGGAACTGCAGTGTCAGCCTGACAGCTC |
| | | TACCCTGCCTCCACCTTGGAGCCCTAGACCTCTGGAAGCTACCGCTCCAACCGCTCCTCAA |
| | | GGGGGACCGTCAGTCTTCCTCTTCCCCCCAAAACCCAAGGACACCCTCTACATCACCCGGG |
| | | AACCTGAGGTCACATGCGTGGTGGTGGACGTGAGCCACGAAGACCCTGAGGTCAAGTTCA |
| | | ACTGGTACGTGGACGGCGTGGAGGTGCATAATGCCAAGACAAAGCCGCGGGAGGAGCAGT |
| | | ACAACAGCACGTACCGTGTGGTCAGCGTCCTCACCGTCCTGCACCAGGACTGGCTGAATGG |
| | | CAAGGAGTACAAGTGCAAGGTCTCCAACAAAGCCCTCCCAGCCCCCATCGAGAAAACCAT |
| | | CTCCAAAGCCAAAGGGCAGCCCCGAGAACCACAGGTGTACACCCTGCCCCCATCCCGGGA |
| | | GGAGATGACCAAGAACCAGGTCAGCCTGACCTGCCTGGTCAAAGGCTTCTATCCCAGCGA |
| | | CATCGCCGTGGAGTGGGAGAGCAATGGGCAGCCGGAGAACAACTACAAGACCACGCCTCC |
| | | CGTGCTGGACTCCGACGGCTCCTTCTTCCTCTACAGCAAGCTCACCGTGGACAAGAGCAGG |
| | | TGGCAGCAGGGGAACGTCTTCTCATGCTCCGTGATGCATGAGGCTCTGCACAACCACTACA |
| | | CGCAGAAGAGCCTCTCCCTGTCTCCGGGTAAA |
| 53 | Aglyco-FLT3L | ACCCAGGACTGCTCCTTCCAGCACTCCCCTATCTCTTCCGACTTCGCCGTGAAGATCAGAG |
| | ECD | AGCTGTCCGACTACCTGCTGCAGGACTATCCTGTGACCGTGGCCAGCAACCTGCAGGATGA |
| | (S128A/S151A) | AGAACTGTGTGGTGGACTGTGGCGACTGGTGCTGGCTCAGAGATGGATGGAAAGACTGAA |
| | hingeless hG1 | AACCGTGGCCGGCTCCAAGATGCAGGGACTGCTGGAAAGAGTGAACACAGAGATCCACTT |
| | (M252Y/S254T/ | CGTGACCAAGTGCGCCTTTCAGCCTCCTCCATCCTGCCTGAGATTCGTGCAGACCAATATC |
| | T256E) | GCCCGGCTGCTGCAAGAGACATCTGAGCAGCTGGTGGCCCTGAAGCCTTGGATCACCAGA |
| | | CAGAACTTCGCCCGGTGCCTGGAACTGCAGTGTCAGCCTGATTCTTCTACCCTGCCTCCACC |
| | | TTGGAGCCCTAGACCTTTGGAGGCTACAGCTCCTACTGCTCCTCAAGGCGGCCCAAGCGTT |
| | | TTCCTGTTTCCTCCAAAGCCTAAGGACACCCTGTACATCACCCGCGAGCCTGAAGTGACAT |
| | | GCGTGGTGGTGGATGTGTCCCACGAGGACCCCGAAGTGAAGTTCAATTGGTACGTGGACG |
| | | GCGTGGAAGTGCACAACGCCAAGACCAAGCCTAGAGAGGAACAGTACAACTCCACCTACA |
| | | GAGTGGTGTCCGTGCTGACCGTGCTGCACCAGGATTGGCTGAACGGCAAAGAGTACAAGT |
| | | GCAAGGTGTCCAACAAGGCCCTGCCTGCTCCTATCGAAAAGACCATCTCCAAGGCCAAGG |
| | | GCCAGCCTAGGGAACCTCAGGTTTACACCCTGCCACCTAGCCGGGAAGAGATGACCAAAA |
| | | ACCAGGTGTCCCTGACCTGCCTGGTCAAGGGCTTCTACCCATCCGATATCGCCGTGGAATG |
| | | GGAGTCTAACGGCCAGCCTGAGAACAACTACAAGACCACACCTCCTGTGCTGGACTCCGA |
| | | CGGCTCATTCTTCCTGTACTCCAAGCTGACAGTGGACAAGTCTCGGTGGCAGCAGGGCAAC |
| | | GTGTTCTCCTGTTCTGTGATGCACGAGGCCCTGCACAACCACTACACCCAGAAGTCCCTGT |
| | | CTCTGTCCCCTGGCAAA |
| 54 | FLT3L ECD (Δ | ACACAGGATTGCAGCTTCCAGCACAGCCCCATCAGCAGCGATTTCGCCGTGAAGATCAGA |
| | C-term 5aa (PTAPQ; | GAGCTGAGCGACTACCTGCTGCAGGACTACCCTGTGACCGTGGCCAGCAATCTGCAGGAC |
| | SEQ ID NO: 85)) - | GAAGAACTGTGTGGTGGACTGTGGCGACTGGTGCTGGCTCAGAGATGGATGGAACGGCTG |
| | hG4 | AAAACAGTGGCCGGCAGCAAGATGCAGGGACTGCTGGAAAGAGTGAACACCGAGATCCA |
| | (S228P/F234A/L23 | CTTCGTGACCAAGTGCGCCTTCCAGCCTCCTCCATCCTGCCTGAGATTCGTGCAGACCAAC |
| | 5A/M252Y/ | ATCAGCAGACTGCTGCAAGAGACAAGCGAGCAGCTGGTGGCCCTGAAGCCTTGGATCACC |
| | S254T/T256E) | AGACAGAACTTCAGCCGGTGCCTGGAACTGCAGTGTCAGCCCGATAGCAGCACACTGCCTC |
| | | CGCCTTGGAGTCCTAGACCTCTGGAAGCCACAGCTGAGTCTAAGTACGGCCCTCCTTGTCC |
| | | TCCATGTCCTGCTCCAGAAGCTGCTGGCGGCCCTTCCGTGTTTCTGTTCCCTCCAAAGCCCA |
| | | AGGACACCCTGTACATCACCCGGGAACCCGAAGTGACCTGCGTGGTGGTGGATGTGTCCCA |
| | | GGAAGATCCCGAGGTGCAGTTCAATTGGTACGTGGACGGCGTGGAAGTGCACAACGCCAA |
| | | GACCAAGCCCAGAGAGGAACAGTTCAACAGCACCTACCGGGTGGTGTCCGTGCTGACAGT |
| | | GCTGCACCAGGACTGGCTGAACGGCAAAGAGTACAAGTGCAAGGTGTCCAACAAGGGCCT |
| | | GCCCAGCTCCATCGAGAAAACCATCAGCAAGGCCAAGGGCCAGCCCCGCGAACCCCAGGT |
| | | GTACACACTGCCTCCAAGCCAGGAAGAGATGACCAAGAACCAGGTGTCCCTGACCTGTCTC |
| | | GTGAAAGGCTTCTACCCCTCCGATATCGCCGTGGAATGGGAGAGCAACGGCCAGCCCGAG |
| | | AACAACTACAAGACCACCCCCCCTGTGCTGGACAGCGACGGCTCATTCTTCCTGTACAGCA |
| | | GACTGACCGTGGACAAGAGCCGGTGGCAGGAAGGCAACGTGTTCAGCTGCAGCGTGATGC |
| | | ACGAGGCCCTGCACAACCACTACACCCAGAAGTCCCTGTCTCTGTCCCTGGGCAAA |
| 55 | FLT3L ECD (Δ | ACCCAGGACTGCTCCTTCCAGCACTCCCCTATCTCTTCCGACTTCGCCGTGAAGATCAGAG |
| | C-term 5aa (PTAPQ; | AGCTGTCCGACTACCTGCTGCAGGACTATCCTGTGACCGTGGCCAGCAACCTGCAGGATGA |
| | SEQ ID NO: 85)) - | AGAACTGTGTGGTGGACTGTGGCGACTGGTGCTGGCTCAGAGATGGATGGAAAGACTGAA |
| | hG4 | AACCGTGGCCGGCTCCAAGATGCAGGGACTGCTGGAAAGAGTGAACACAGAGATCCACTT |
| | (S228P/F234A/L23 | CGTGACCAAGTGCGCCTTTCAGCCTCCTCCATCCTGCCTGAGATTCGTGCAGACCAACATCT |
| | 5A/M252Y/ | CCCGGCTGCTGCAAGAGACATCTGAGCAGCTGGTGGCCCTGAAGCCTTGGATCACCCGGCA |
| | S254T/T256E) | GAACTTCTCTCGGTGCCTGGAACTGCAGTGTCAGCCTGATTCTTCTACCCTGCCTCCACCTT |
| | | GGAGCCCTAGACCTCTGGAAGCTACCGCCGAGTCTAAGTACGGACCTCCTTGTCCTCCATG |
| | | TCCTGCTCCAGAAGCTGCTGGCGGACCAAGCGTTTTCCTGTTTCCTCCAAAGCCTAAGGAC |
| | | ACCCTGTACATCACCCGCGAGCCTGAAGTGACATGCGTGGTGGTGGATGTGTCCCAAGAGG |
| | | ACCCCGAGGTGCAGTTCAATTGGTACGTGGACGGCGTGGAAGTGCACAACGCCAAGACCA |
| | | AGCCTAGAGAGGAACAGTTCAACTCCACCTACAGAGTGGTGTCCGTGCTGACCGTGCTGCA |
| | | CCAGGATTGGCTGAACGGCAAAGAGTACAAGTGCAAGGTGTCCAACAAGGGCCTGCCTAG |
| | | CTCCATCGAAAAGACCATCTCCAAGGCCAAGGGCCAGCCAAGAGAACCTCAGGTGTACAC |
| | | ACTGCCTCCAAGCCAAGAGGAAATGACCAAGAACCAGGTGTCCCTGACCTGCCTGGTCAA |
| | | GGGCTTCTACCCATCCGATATCGCCGTGGAATGGGAGTCTAACGGCCAGCCTGAGAACAAC |
| | | TACAAGACCACACCTCCTGTGCTGGACTCCGACGGCTCCTTCTTTCTGTACTCTCGCCTGAC |
| | | CGTGGACAAGTCTAGATGGCAAGAGGGCAACGTGTTCTCCTGCTCTGTGATGCACGAGGCC |
| | | CTGCACAACCACTACACCCAGAAGTCCCTGTCTCTGTCCCTGGGCAAA |
| 56 | FLT3L ECD (Δ | ACACAGGATTGCAGCTTCCAGCACAGCCCCATCAGCAGCGATTTCGCCGTGAAGATCAGA |
| | C-term 10aa | GAGCTGAGCGACTACCTGCTGCAGGACTACCCTGTGACCGTGGCCAGCAATCTGCAGGAC |
| | (LEATAPTAPQ; | GAAGAACTGTGTGGTGGACTGTGGCGACTGGTGCTGGCTCAGAGATGGATGGAACGGCTG |
| | SEQ ID NO: 90))- | AAAACAGTGGCCGGCAGCAAGATGCAGGGACTGCTGGAAAGAGTGAACACCGAGATCCA |
| | hG1 | CTTCGTGACCAAGTGCGCCTTCCAGCCTCCTCCATCCTGCCTGAGATTCGTGCAGACCAAC |
| | (M252Y/S254T/ | ATCAGCAGACTGCTGCAAGAGACAAGCGAGCAGCTGGTGGCCCTGAAGCCTTGGATCACC |
| | T256E) | AGACAGAACTTCAGCCGGTGCCTGGAACTGCAGTGTCAGCCCGATAGCAGCACACTGCCTC |
| | | CGCCTTGGAGTCCTAGACCTGGGGGACCGTCAGTCTTCCTCTTCCCCCCAAAACCCAAGGA |
| | | CACCCTCTACATCACCCGGGAACCTGAGGTCACATGCGTGGTGGTGGACGTGAGCCACGA |
| | | AGACCCTGAGGTCAAGTTCAACTGGTACGTGGACGGCGTGGAGGTGCATAATGCCAAGAC |
| | | AAAGCCGCGGGAGGAGCAGTACAACAGCACGTACCGTGTGGTCAGCGTCCTCACCGTCCT |
| | | GCACCAGGACTGGCTGAATGGCAAGGAGTACAAGTGCAAGGTCTCCAACAAAGCCCTCCC |
| | | AGCCCCCATCGAGAAAACCATCTCCAAAGCCAAAGGGCAGCCCCGAGAACCACAGGTGTA |
| | | CACCCTGCCCCCATCCCGGGAGGAGATGACCAAGAACCAGGTCAGCCTGACCTGCCTGGTC |
| | | AAAGGCTTCTATCCCAGCGACATCGCCGTGGAGTGGGAGAGCAATGGGCAGCCGGAGAAC |
| | | AACTACAAGACCACGCCTCCCGTGCTGGACTCCGACGGCTCCTTCTTCCTCTACAGCAAGC |
| | | TCACCGTGGACAAGAGCAGGTGGCAGCAGGGGAACGTCTTCTCATGCTCCGTGATGCATG |
| | | AGGCTCTGCACAACCACTACACGCAGAAGAGCCTCTCCCTGTCTCCGGGTAAA |
| 57 | FLT3L ECD (Δ | ACCCAGGACTGCTCCTTCCAGCACTCCCCTATCTCTTCCGACTTCGCCGTGAAGATCAGAG |
| | C-term 10aa | AGCTGTCCGACTACCTGCTGCAGGACTATCCTGTGACCGTGGCCAGCAACCTGCAGGATGA |
| | (LEATAPTAPQ; | AGAACTGTGTGGTGGACTGTGGCGACTGGTGCTGGCTCAGAGATGGATGGAAAGACTGAA |
| | SEQ ID NO: 90))- | AACCGTGGCCGGCTCCAAGATGCAGGGACTGCTGGAAAGAGTGAACACAGAGATCCACTT |
| | hG1 | CGTGACCAAGTGCGCCTTTCAGCCTCCTCCATCCTGCCTGAGATTCGTGCAGACCAACATCT |
| | (M252Y/S254T/ | CCCGGCTGCTGCAAGAGACATCTGAGCAGCTGGTGGCCCTGAAGCCTTGGATCACCCGGCA |
| | T256E) | GAACTTCTCTCGGTGCCTGGAACTGCAGTGTCAGCCTGATTCTTCTACCCTGCCTCCACCTT |
| | | GGAGTCCTAGACCTGGCGGACCAAGCGTGTTCCTGTTTCCTCCAAAGCCTAAGGACACCCT |
| | | GTACATCACCCGCGAGCCTGAAGTGACATGCGTGGTGGTGGATGTGTCCCACGAGGACCCC |
| | | GAAGTGAAGTTCAATTGGTACGTGGACGGCGTGGAAGTGCACAACGCCAAGACCAAGCCT |
| | | AGAGAGGAACAGTACAACTCCACCTACAGAGTGGTGTCCGTGCTGACCGTGCTGCACCAG |
| | | GATTGGCTGAACGGCAAAGAGTACAAGTGCAAGGTGTCCAACAAGGCCCTGCCTGCTCCT |
| | | ATCGAAAAGACCATCTCCAAGGCCAAGGGCCAGCCTAGGGAACCTCAGGTTTACACCCTG |
| | | CCACCTAGCCGGGAAGAGATGACCAAAAACCAGGTGTCCCTGACCTGCCTGGTCAAGGGC |
| | | TTCTACCCATCCGATATCGCCGTGGAATGGGAGTCTAACGGCCAGCCTGAGAACAACTACA |
| | | AGACCACACCTCCTGTGCTGGACTCCGACGGCTCATTCTTCCTGTACTCCAAGCTGACAGT |
| | | GGACAAGTCTCGGTGGCAGCAGGGCAACGTGTTCTCCTGTTCTGTGATGCACGAGGCCCTG |
| | | CACAACCACTACACCCAGAAGTCCCTGTCTCTGTCCCCTGGCAAA |
| 58 | FLT3L ECD | ACACAGGATTGCAGCTTCCAGCACAGCCCCATCAGCAGCGATTTCGCCGTGAAGATCAGA |
| | (Δ C-term 10aa | GAGCTGAGCGACTACCTGCTGCAGGACTACCCTGTGACCGTGGCCAGCAATCTGCAGGAC |
| | (LEATAPTAPQ; | GAAGAACTGTGTGGTGGACTGTGGCGACTGGTGCTGGCTCAGAGATGGATGGAACGGCTG |
| | SEQ ID NO: 90))- | AAAACAGTGGCCGGCAGCAAGATGCAGGGACTGCTGGAAAGAGTGAACACCGAGATCCA |
| | hG4 | CTTCGTGACCAAGTGCGCCTTCCAGCCTCCTCCATCCTGCCTGAGATTCGTGCAGACCAAC |
| | (S228P/F234A/ | ATCAGCAGACTGCTGCAAGAGACAAGCGAGCAGCTGGTGGCCCTGAAGCCTTGGATCACC |
| | L235A/M252Y/ | AGACAGAACTTCAGCCGGTGCCTGGAACTGCAGTGTCAGCCCGATAGCAGCACACTGCCTC |
| | S254T/T256E) | CGCCTTGGAGTCCTAGACCTGAGTCTAAGTACGGCCCTCCTTGTCCTCCATGTCCTGCTCCA |
| | | GAAGCTGCTGGCGGCCCTTCCGTGTTTCTGTTCCCTCCAAAGCCTAAGGACACCCTGTACAT |
| | | CACCCGGGAACCCGAAGTGACCTGCGTGGTGGTGGATGTGTCCCAGGAAGATCCCGAGGT |
| | | GCAGTTCAATTGGTACGTGGACGGCGTGGAAGTGCACAACGCCAAGACCAAGCCCAGAGA |
| | | GGAACAGTTCAACAGCACCTACCGGGTGGTGTCCGTGCTGACAGTGCTGCACCAGGACTG |
| | | GCTGAACGGCAAAGAGTACAAGTGCAAGGTGTCCAACAAGGGCCTGCCCAGCTCCATCGA |
| | | GAAAACCATCAGCAAGGCCAAGGGCCAGCCCCGCGAACCCCAGGTGTACACACTGCCTCC |
| | | AAGCCAGGAAGAGATGACCAAGAACCAGGTGTCCCTGACCTGTCTCGTGAAAGGCTTCTA |
| | | CCCCTCCGATATCGCCGTGGAATGGGAGAGCAACGGCCAGCCCGAGAACAACTACAAGAC |
| | | CACCCCCCCTGTGCTGGACAGCGACGGCTCATTCTTCCTGTACAGCAGACTGACCGTGGAC |
| | | AAGAGCCGGTGGCAGGAAGGCAACGTGTTCAGCTGCAGCGTGATGCACGAGGCCCTGCAC |
| | | AACCACTACACCCAGAAGTCCCTGTCTCTGTCCCTGGGCAAA |
| 59 | FLT3L ECD | ACCCAGGACTGCTCCTTCCAGCACTCCCCTATCTCTTCCGACTTCGCCGTGAAGATCAGAG |
| | (Δ C-term 10aa | AGCTGTCCGACTACCTGCTGCAGGACTATCCTGTGACCGTGGCCAGCAACCTGCAGGATGA |
| | (LEATAPTAPQ; | AGAACTGTGTGGTGGACTGTGGCGACTGGTGCTGGCTCAGAGATGGATGGAAAGACTGAA |
| | SEQ ID NO: 90))- | AACCGTGGCCGGCTCCAAGATGCAGGGACTGCTGGAAAGAGTGAACACAGAGATCCACTT |
| | hG4 | CGTGACCAAGTGCGCCTTTCAGCCTCCTCCATCCTGCCTGAGATTCGTGCAGACCAACATCT |
| | (S228P/F234A/ | CCCGGCTGCTGCAAGAGACATCTGAGCAGCTGGTGGCCCTGAAGCCTTGGATCACCCGGCA |
| | L235A/M252Y/ | GAACTTCTCTCGGTGCCTGGAACTGCAGTGTCAGCCTGATTCTTCTACCCTGCCTCCACCTT |
| | S254T/T256E) | GGAGCCCTCGGCCTGAATCTAAGTATGGCCCTCCTTGTCCTCCATGTCCTGCTCCAGAAGCT |
| | | GCTGGCGGACCAAGCGTTTTCCTGTTTCCTCCAAAGCCTAAGGACACCCTGTACATCACCC |
| | | GCGAGCCTGAAGTGACATGCGTGGTGGTGGATGTGTCCCAAGAGGACCCCGAGGTGCAGT |
| | | TCAATTGGTACGTGGACGGCGTGGAAGTGCACAACGCCAAGACCAAGCCTAGAGAGGAAC |
| | | AGTTCAACTCCACCTACAGAGTGGTGTCCGTGCTGACCGTGCTGCACCAGGATTGGCTGAA |
| | | CGGCAAAGAGTACAAGTGCAAGGTGTCCAACAAGGGCCTGCCTAGCTCCATCGAAAAGAC |
| | | CATCTCCAAGGCCAAGGGCCAGCCAAGAGAACCTCAGGTGTACACACTGCCTCCAAGCCA |
| | | AGAGGAAATGACCAAGAACCAGGTGTCCCTGACCTGCCTGGTCAAGGGCTTCTACCCATCC |
| | | GATATCGCCGTGGAATGGGAGTCTAACGGCCAGCCTGAGAACAACTACAAGACCACACCT |
| | | CCTGTGCTGGACTCCGACGGCTCCTTCTTTCTGTACTCTCGCCTGACCGTGGACAAGTCTAG |
| | | ATGGCAAGAGGGCAACGTGTTCTCCTGCTCTGTGATGCACGAGGCCCTGCACAACCACTAC |
| | | ACCCAGAAGTCCCTGTCTCTGTCCCTGGGCAAA |
| 60 | Aglyco-FLT3L | ACCCAGGACTGTTCCTTCCAGCACTCCCCTATCTCCAGCGACTTCGCCGTGAAGATCAGAG |
| | ECD (Δ C-term | AGCTGTCCGACTATCTGCTGCAGGACTACCCTGTGACCGTGGCCAGCAATCTGCAGGACGA |
| | 10aa | AGAACTGTGTGGTGGCCTGTGGCGACTGGTGTTGGCTCAGAGATGGATGGAACGGCTGAA |
| | (LEATAPTAPQ; | AACCGTGGCCGGCTCTAAGATGCAGGGCCTGCTGGAAAGAGTGAACACCGAGATCCACTT |
| | SEQ ID NO: 90)) | CGTGACCAAGTGCGCCTTTCAGCCTCCTCCATCCTGCCTGAGATTCGTGCAGACCAATATC |
| | (S128A/S151A)- | GCCCGGCTGCTGCAAGAGACATCCGAGCAGCTGGTGGCTCTGAAGCCCTGGATCACCAGA |
| | hG4 S228P/F234A/ | CAGAACTTCGCCCGGTGTCTGGAACTGCAGTGTCAGCCTGACAGCTCTACCCTGCCTCCAC |
| | L235A | CTTGGAGCCCTAGACCTGAGTCTAAGTACGGCCCTCCTTGTCCTCCATGTCCTGCTCCAGAA |
| | | GCTGCTGGCGGCCCTTCCGTGTTTCTGTTCCCTCCAAAGCCTAAGGACACCCTGATGATCTC |
| | | TCGGACCCCTGAAGTGACCTGCGTGGTGGTGGATGTGTCCCAAGAGGATCCCGAGGTGCA |
| | | GTTCAATTGGTACGTGGACGGCGTGGAAGTGCACAACGCCAAGACCAAGCCTAGAGAGGA |
| | | ACAGTTCAACTCCACCTACAGAGTGGTGTCCGTGCTGACCGTGCTGCACCAGGATTGGCTG |
| | | AACGGCAAAGAGTACAAGTGCAAGGTGTCCAACAAGGGCCTGCCTTCCAGCATCGAAAAG |
| | | ACCATCTCCAAGGCCAAGGGCCAGCCTAGGGAACCCCAGGTTTACACCCTGCCTCCAAGCC |
| | | AAGAGGAAATGACCAAGAACCAGGTGTCCCTGACCTGCCTGGTCAAGGGCTTCTACCCTTC |
| | | CGATATCGCCGTGGAATGGGAGAGCAATGGCCAGCCTGAGAACAACTACAAGACCACACC |
| | | TCCTGTGCTGGACTCCGACGGCTCCTTCTTTCTGTACTCCCGCCTGACCGTGGACAAGTCCA |
| | | GATGGCAAGAGGGCAACGTGTTCTCCTGCTCCGTGATGCACGAGGCCCTGCACAATCACTA |
| | | CACCCAGAAGTCCCTGTCTCTGTCCCTGGGCAAA |
| 61 | Aglyco-FLT3L | ACCCAGGACTGTTCCTTCCAGCACTCCCCTATCTCCAGCGACTTCGCCGTGAAGATCAGAG |
| | ECD (Δ C-term | AGCTGTCCGACTATCTGCTGCAGGACTACCCTGTGACCGTGGCCAGCAATCTGCAGGACGA |
| | 10aa | AGAACTGTGTGGTGGCCTGTGGCGACTGGTGTTGGCTCAGAGATGGATGGAACGGCTGAA |
| | (LEATAPTAPQ; | AACCGTGGCCGGCTCTAAGATGCAGGGCCTGCTGGAAAGAGTGAACACCGAGATCCACTT |
| | SEQ ID NO: 90)) | CGTGACCAAGTGCGCCTTTCAGCCTCCTCCATCCTGCCTGAGATTCGTGCAGACCAATATC |
| | (S128A/S151A)- | GCCCGGCTGCTGCAAGAGACATCCGAGCAGCTGGTGGCTCTGAAGCCCTGGATCACCAGA |
| | hG4 | CAGAACTTCGCCCGGTGTCTGGAACTGCAGTGTCAGCCTGACAGCTCTACCCTGCCTCCAC |
| | (S228P/F234A/ | CTTGGAGCCCTAGACCTGAGTCTAAGTACGGCCCTCCTTGTCCTCCATGTCCTGCTCCAGAA |
| | L235A/M252Y/ | GCTGCTGGCGGCCCTTCCGTGTTTCTGTTCCCTCCAAAGCCCAAGGACACCCTGTACATCAC |
| | S254T/T256E) | CCGGGAACCCGAAGTGACCTGCGTGGTGGTGGATGTGTCCCAGGAAGATCCCGAGGTGCA |
| | | GTTCAATTGGTACGTGGACGGCGTGGAAGTGCACAACGCCAAGACCAAGCCCAGAGAGGA |
| | | ACAGTTCAACAGCACCTACCGGGTGGTGTCCGTGCTGACAGTGCTGCACCAGGACTGGCTG |
| | | AACGGCAAAGAGTACAAGTGCAAGGTGTCCAACAAGGGCCTGCCCAGCTCCATCGAGAAA |
| | | ACCATCAGCAAGGCCAAGGGCCAGCCCCGCGAACCCCAGGTGTACACACTGCCTCCAAGC |
| | | CAGGAAGAGATGACCAAGAACCAGGTGTCCCTGACCTGTCTCGTGAAAGGCTTCTACCCCT |
| | | CCGATATCGCCGTGGAATGGGAGAGCAACGGCCAGCCCGAGAACAACTACAAGACCACCC |
| | | CCCCTGTGCTGGACAGCGACGGCTCATTCTTCCTGTACAGCAGACTGACCGTGGACAAGAG |
| | | CCGGTGGCAGGAAGGCAACGTGTTCAGCTGCAGCGTGATGCACGAGGCCCTGCACAACCA |
| | | CTACACCCAGAAGTCCCTGTCTCTGTCCCTGGGCAAA |
| 62 | Murine surrogate | ACCCCTGACTGCTACTTCAGCCACTCTCCTATCTCCAGCAACTTCAAAGTGAAGTTCCGCGA |
| | mFLT3L ECD - | GCTGACCGACCATCTGCTGAAGGACTATCCTGTGACCGTGGCCGTGAACCTGCAGGACGAA |
| | mG2aFc | AAGCACTGCAAGGCCCTGTGGTCCCTGTTTCTGGCCCAGAGATGGATCGAGCAGCTGAAAA |
| | (L234A/L235A/ | CCGTGGCTGGCTCCAAGATGCAGACCCTGCTGGAAGATGTGAACACCGAGATCCACTTCGT |
| | P329G) | GACCAGCTGCACCTTCCAGCCTCTGCCTGAGTGCCTGAGATTCGTGCAGACCAACATCTCC |
| | | CACCTGTTGAAGGACACATGCACCCAGCTGCTGGCCCTGAAGCCTTGTATCGGCAAGGCCT |
| | | GCCAGAACTTCTCCCGGTGTCTGGAAGTGCAGTGCCAGCCTGACTCCTCCACACTGCTGCC |
| | | ACCTAGAAGCCCTATCGCTCTGGAAGCTACCGAGCTGCCTGAGCCTAGAGGCCCTACCATC |
| | | AAGCCTTGTCCTCCATGCAAGTGCCCCGCTCCTAATGCTGCTGGTGGCCCTTCCGTGTTCAT |
| | | CTTCCCACCTAAGATCAAGGACGTGCTGATGATCTCCCTGTCTCCTATCGTGACCTGCGTGG |
| | | TGGTGGACGTGTCCGAGGATGATCCTGACGTGCAGATCAGTTGGTTCGTGAACAACGTGGA |
| | | AGTGCACACCGCTCAGACCCAGACACACAGAGAGGACTACAACAGCACCCTGAGAGTGGT |
| | | GTCTGCCCTGCCTATCCAGCACCAGGATTGGATGTCCGGCAAAGAATTCAAGTGCAAAGTG |
| | | AACAACAAGGACCTGGGCGCTCCCATCGAGCGGACCATCTCTAAGCCTAAGGGATCCGTC |
| | | AGAGCCCCTCAGGTGTACGTTCTGCCTCCACCTGAGGAAGAGATGACCAAGAAACAAGTG |
| | | ACCCTGACCTGCATGGTCACCGACTTCATGCCCGAGGACATCTACGTGGAATGGACCAACA |
| | | ACGGCAAGACCGAGCTGAACTACAAGAACACCGAGCCTGTGCTGGACTCCGACGGCTCCT |
| | | ACTTCATGTACTCCAAGCTGCGCGTCGAGAAGAAGAACTGGGTCGAGAGAAACTCCTACTC |
| | | CTGCTCCGTGGTGCACGAGGGCCTGCACAATCACCACACCACCAAGTCCTTCTCTCGGACC |
| | | CCTGGCAAA |
| 63 | Murine surrogate | ACCCCTGACTGCTACTTCAGCCACTCTCCTATCTCCAGCAACTTCAAAGTGAAGTTCCGCGA |
| | mFLT3L ECD | GCTGACCGACCATCTGCTGAAGGACTATCCTGTGACCGTGGCCGTGAACCTGCAGGACGAA |
| | (C136S) mG2aFc | AAGCACTGCAAGGCCCTGTGGTCCCTGTTTCTGGCCCAGAGATGGATCGAGCAGCTGAAAA |
| | (L234A/L235A/ | CCGTGGCTGGCTCCAAGATGCAGACCCTGCTGGAAGATGTGAACACCGAGATCCACTTCGT |
| | P329G) | GACCAGCTGCACCTTCCAGCCTCTGCCTGAGTGCCTGAGATTCGTGCAGACCAACATCTCC |
| | | CACCTGTTGAAGGACACATCCACCCAGCTGCTGGCCCTGAAGCCTTGTATCGGCAAGGCCT |
| | | GCCAGAACTTCTCCCGGTGTCTGGAAGTGCAGTGCCAGCCTGACTCCTCCACACTGCTGCC |
| | | ACCTAGAAGCCCTATCGCTCTGGAAGCTACCGAGCTGCCTGAGCCTAGAGGCCCTACCATC |
| | | AAGCCTTGTCCTCCATGCAAGTGCCCCGCTCCTAATGCTGCTGGTGGCCCTTCCGTGTTCAT |
| | | CTTCCCACCTAAGATCAAGGACGTGCTGATGATCTCCCTGTCTCCTATCGTGACCTGCGTGG |
| | | TGGTGGACGTGTCCGAGGATGATCCTGACGTGCAGATCAGTTGGTTCGTGAACAACGTGGA |
| | | AGTGCACACCGCTCAGACCCAGACACACAGAGAGGACTACAACAGCACCCTGAGAGTGGT |
| | | GTCTGCCCTGCCTATCCAGCACCAGGATTGGATGTCCGGCAAAGAATTCAAGTGCAAAGTG |
| | | AACAACAAGGACCTGGGCGCTCCCATCGAGCGGACCATCTCTAAGCCTAAGGGATCCGTC |
| | | AGAGCCCCTCAGGTGTACGTTCTGCCTCCACCTGAGGAAGAGATGACCAAGAAACAAGTG |
| | | ACCCTGACCTGCATGGTCACCGACTTCATGCCCGAGGACATCTACGTGGAATGGACCAACA |
| | | ACGGCAAGACCGAGCTGAACTACAAGAACACCGAGCCTGTGCTGGACTCCGACGGCTCCT |
| | | ACTTCATGTACTCCAAGCTGCGCGTCGAGAAGAAGAACTGGGTCGAGAGAAACTCCTACTC |
| | | CTGCTCCGTGGTGCACGAGGGCCTGCACAATCACCACACCACCAAGTCCTTCTCTCGGACC |
| | | CCTGGCAAA |
| 64 | FLT3L ECD- | ACACAGGATTGCAGCTTCCAGCACAGCCCCATCAGCAGCGATTTCGCCGTGAAGATCAGA |
| | hingeless monoFc | GAGCTGAGCGACTACCTGCTGCAGGACTACCCTGTGACCGTGGCCAGCAATCTGCAGGAC |
| | | GAAGAACTGTGTGGTGGACTGTGGCGACTGGTGCTGGCTCAGAGATGGATGGAACGGCTG |
| | | AAAACAGTGGCCGGCAGCAAGATGCAGGGACTGCTGGAAAGAGTGAACACCGAGATCCA |
| | | CTTCGTGACCAAGTGCGCCTTCCAGCCTCCTCCATCCTGCCTGAGATTCGTGCAGACCAAC |
| | | ATCAGCAGACTGCTGCAAGAGACAAGCGAGCAGCTGGTGGCCCTGAAGCCTTGGATCACC |
| | | AGACAGAACTTCAGCCGGTGCCTGGAACTGCAGTGTCAGCCCGATAGCAGCACACTGCCTC |
| | | CGCCTTGGAGTCCTAGACCTCTGGAAGCCACAGCTCCCACCGCTCCTCAAGGCGGACCGTC |
| | | AGTCTTTCTGTTCCCTCCAAAGCCTAAGGACACCCTGATGATCAGCAGAACCCCTGAAGTG |
| | | ACCTGCGTGGTGGTGGATGTGTCCCACGAGGATCCCGAAGTGAAGTTCAATTGGTACGTGG |
| | | ACGGCGTGGAAGTGCACAACGCCAAGACCAAGCCTAGAGAGGAACAGTACAACAGCACCT |
| | | ACAGAGTGGTGTCCGTGCTGACCGTGCTGCACCAGGATTGGCTGAACGGCAAAGAGTACA |
| | | AGTGCAAGGTGTCCAACAAGGCCCTGCCTGCTCCTATCGAGAAAACCATCAGCAAGGCCA |
| | | AGGGCCAGCCTAGGGAACCCCAGGTGTACACAAAGCCTCCAAGCCGGGAAGAGATGACCA |
| | | AGAACCAGGTGTCCCTGAGCTGCCTGGTCAAGGGCTTTTACCCCAGCGACATTGCCGTGGA |
| | | ATGGGAGAGCAATGGCCAGCCTGAGAACAACTACAAGACCACCGTGCCTGTGCTGGACAG |
| | | CGACGGCTCTTTTAGACTGGCCAGCTACCTGACCGTGGACAAGAGCAGATGGCAGCAGGG |
| | | CAACGTGTTCAGCTGCAGCGTGATGCACGAGGCCCTGCACAACCACTACACCCAGAAGTCC |
| | | CTGTCTCTGAGCCCCGGCAAA |
| 65 | FLT3L ECD (H8Y) - | ACACAGGATTGCAGCTTCCAGTACAGCCCCATCAGCAGCGATTTCGCCGTGAAGATCAGAG |
| | hingeless hG1 Fc | AGCTGAGCGACTACCTGCTGCAGGACTACCCTGTGACCGTGGCCAGCAATCTGCAGGACG |
| | | AAGAACTGTGTGGTGGACTGTGGCGACTGGTGCTGGCTCAGAGATGGATGGAACGGCTGA |
| | | AAACAGTGGCCGGCAGCAAGATGCAGGGACTGCTGGAAAGAGTGAACACCGAGATCCACT |
| | | TCGTGACCAAGTGCGCCTTCCAGCCTCCTCCATCCTGCCTGAGATTCGTGCAGACCAACAT |
| | | CAGCAGACTGCTGCAAGAGACAAGCGAGCAGCTGGTGGCCCTGAAGCCTTGGATCACCAG |
| | | ACAGAACTTCAGCCGGTGCCTGGAACTGCAGTGTCAGCCCGATAGCAGCACACTGCCTCCG |
| | | CCTTGGAGTCCTAGACCTCTGGAAGCCACAGCTCCCACCGCTCCTCAAGGCGGACCGTCAG |
| | | TCTTCCTCTTCCCCCCAAAACCCAAGGACACCCTCATGATCTCCCGGACCCCTGAGGTCAC |
| | | ATGCGTGGTGGTGGACGTGAGCCACGAAGACCCTGAGGTCAAGTTCAACTGGTACGTGGA |
| | | CGGCGTGGAGGTGCATAATGCCAAGACAAAGCCGCGGGAGGAGCAGTACAACAGCACGT |
| | | ACCGTGTGGTCAGCGTCCTCACCGTCCTGCACCAGGACTGGCTGAATGGCAAGGAGTACAA |
| | | GTGCAAGGTCTCCAACAAAGCCCTCCCAGCCCCCATCGAGAAAACCATCTCCAAAGCCAA |
| | | AGGGCAGCCCCGAGAACCACAGGTGTACACCCTGCCCCCATCCCGGGAGGAGATGACCAA |
| | | GAACCAGGTCAGCCTGACCTGCCTGGTCAAAGGCTTCTATCCCAGCGACATCGCCGTGGAG |
| | | TGGGAGAGCAATGGGCAGCCGGAGAACAACTACAAGACCACGCCTCCCGTGCTGGACTCC |
| | | GACGGCTCCTTCTTCCTCTACAGCAAGCTCACCGTGGACAAGAGCAGGTGGCAGCAGGGG |
| | | AACGTCTTCTCATGCTCCGTGATGCATGAGGCTCTGCACAACCACTACACGCAGAAGAGCC |
| | | TCTCCCTGTCTCCGGGTAAA |
| 66 | FLT3L ECD | ACACAGGATTGCAGCTTCCAGCACAGCCCCATCAGCAGCGATTTCGCCGTGAAGATCAGA |
| | (K84E) - hingeless | GAGCTGAGCGACTACCTGCTGCAGGACTACCCTGTGACCGTGGCCAGCAATCTGCAGGAC |
| | hG1 Fc | GAAGAACTGTGTGGTGGACTGTGGCGACTGGTGCTGGCTCAGAGATGGATGGAACGGCTG |
| | | AAAACAGTGGCCGGCAGCAAGATGCAGGGACTGCTGGAAAGAGTGAACACCGAGATCCA |
| | | CTTCGTGACCGAGTGCGCCTTCCAGCCTCCTCCATCCTGCCTGAGATTCGTGCAGACCAAC |
| | | ATCAGCAGACTGCTGCAAGAGACAAGCGAGCAGCTGGTGGCCCTGAAGCCTTGGATCACC |
| | | AGACAGAACTTCAGCCGGTGCCTGGAACTGCAGTGTCAGCCCGATAGCAGCACACTGCCTC |
| | | CGCCTTGGAGTCCTAGACCTCTGGAAGCCACAGCTCCCACCGCTCCTCAAGGCGGACCGTC |
| | | AGTCTTCCTCTTCCCCCCAAAACCCAAGGACACCCTCATGATCTCCCGGACCCCTGAGGTC |
| | | ACATGCGTGGTGGTGGACGTGAGCCACGAAGACCCTGAGGTCAAGTTCAACTGGTACGTG |
| | | GACGGCGTGGAGGTGCATAATGCCAAGACAAAGCCGCGGGAGGAGCAGTACAACAGCAC |
| | | GTACCGTGTGGTCAGCGTCCTCACCGTCCTGCACCAGGACTGGCTGAATGGCAAGGAGTAC |
| | | AAGTGCAAGGTCTCCAACAAAGCCCTCCCAGCCCCCATCGAGAAAACCATCTCCAAAGCC |
| | | AAAGGGCAGCCCCGAGAACCACAGGTGTACACCCTGCCCCCATCCCGGGAGGAGATGACC |
| | | AAGAACCAGGTCAGCCTGACCTGCCTGGTCAAAGGCTTCTATCCCAGCGACATCGCCGTGG |
| | | AGTGGGAGAGCAATGGGCAGCCGGAGAACAACTACAAGACCACGCCTCCCGTGCTGGACT |
| | | CCGACGGCTCCTTCTTCCTCTACAGCAAGCTCACCGTGGACAAGAGCAGGTGGCAGCAGGG |
| | | GAACGTCTTCTCATGCTCCGTGATGCATGAGGCTCTGCACAACCACTACACGCAGAAGAGC |
| | | CTCTCCCTGTCTCCGGGTAAA |
| 67 | FLT3L ECD | ACACAGGATTGCAGCTTCCAGTACAGCCCCATCAGCAGCGATTTCGCCGTGAAGATCAGAG |
| | (H8Y/K84E) | AGCTGAGCGACTACCTGCTGCAGGACTACCCTGTGACCGTGGCCAGCAATCTGCAGGACG |
| | hingeless hG1 Fc | AAGAACTGTGTGGTGGACTGTGGCGACTGGTGCTGGCTCAGAGATGGATGGAACGGCTGA |
| | | AAACAGTGGCCGGCAGCAAGATGCAGGGACTGCTGGAAAGAGTGAACACCGAGATCCACT |
| | | TCGTGACCGAGTGCGCCTTCCAGCCTCCTCCATCCTGCCTGAGATTCGTGCAGACCAACAT |
| | | CAGCAGACTGCTGCAAGAGACAAGCGAGCAGCTGGTGGCCCTGAAGCCTTGGATCACCAG |
| | | ACAGAACTTCAGCCGGTGCCTGGAACTGCAGTGTCAGCCCGATAGCAGCACACTGCCTCCG |
| | | CCTTGGAGTCCTAGACCTCTGGAAGCCACAGCTCCCACCGCTCCTCAAGGCGGACCGTCAG |
| | | TCTTCCTCTTCCCCCCAAAACCCAAGGACACCCTCATGATCTCCCGGACCCCTGAGGTCAC |
| | | ATGCGTGGTGGTGGACGTGAGCCACGAAGACCCTGAGGTCAAGTTCAACTGGTACGTGGA |
| | | CGGCGTGGAGGTGCATAATGCCAAGACAAAGCCGCGGGAGGAGCAGTACAACAGCACGT |
| | | ACCGTGTGGTCAGCGTCCTCACCGTCCTGCACCAGGACTGGCTGAATGGCAAGGAGTACAA |
| | | GTGCAAGGTCTCCAACAAAGCCCTCCCAGCCCCCATCGAGAAAACCATCTCCAAAGCCAA |
| | | AGGGCAGCCCCGAGAACCACAGGTGTACACCCTGCCCCCATCCCGGGAGGAGATGACCAA |
| | | GAACCAGGTCAGCCTGACCTGCCTGGTCAAAGGCTTCTATCCCAGCGACATCGCCGTGGAG |
| | | TGGGAGAGCAATGGGCAGCCGGAGAACAACTACAAGACCACGCCTCCCGTGCTGGACTCC |
| | | GACGGCTCCTTCTTCCTCTACAGCAAGCTCACCGTGGACAAGAGCAGGTGGCAGCAGGGG |
| | | AACGTCTTCTCATGCTCCGTGATGCATGAGGCTCTGCACAACCACTACACGCAGAAGAGCC |
| | | TCTCCCTGTCTCCGGGTAAA |
| 68 | Aglyco-FLT3L | ACCCAGGACTGTTCCTTCCAGCACTCCCCTATCTCCAGCGACTTCGCCGTGAAGATCAGAG |
| | ECD (Δ C-term 5aa | AGCTGTCCGACTATCTGCTGCAGGACTACCCTGTGACCGTGGCCAGCAATCTGCAGGACGA |
| | (PTAPQ; SEQ ID | AGAACTGTGTGGTGGCCTGTGGCGACTGGTGTTGGCTCAGAGATGGATGGAACGGCTGAA |
| | NO: 85)) | AACCGTGGCCGGCTCTAAGATGCAGGGCCTGCTGGAAAGAGTGAACACCGAGATCCACTT |
| | (S128A/S151A) - | CGTGACCAAGTGCGCCTTTCAGCCTCCTCCATCCTGCCTGAGATTCGTGCAGACCAATATC |
| | hG4 | GCCCGGCTGCTGCAAGAGACATCCGAGCAGCTGGTGGCTCTGAAGCCCTGGATCACCAGA |
| | (S228P/F234A/ | CAGAACTTCGCCCGGTGTCTGGAACTGCAGTGTCAGCCTGACAGCTCTACCCTGCCTCCAC |
| | L235A/M252Y/S254T/ | CTTGGAGCCCTAGACCTCTGGAAGCTACCGCTGAGTCTAAGTACGGCCCTCCTTGTCCTCC |
| | T256E) | ATGTCCTGCTCCAGAAGCTGCTGGCGGCCCTTCCGTGTTTCTGTTCCCTCCAAAGCCcAAGG |
| | | ACACCCTGtacATCaccCGGgaaCCCGAAGTGACCTGCGTGGTGGTGGATGTGTCCCAGGAAGA |
| | | TCCCGAGGTGCAGTTCAATTGGTACGTGGACGGCGTGGAAGTGCACAACGCCAAGACCAA |
| | | GCCCAGAGAGGAACAGTTCAACAGCACCTACCGGGTGGTGTCCGTGCTGACAGTGCTGCA |
| | | CCAGGACTGGCTGAACGGCAAAGAGTACAAGTGCAAGGTGTCCAACAAGGGCCTGCCCAG |
| | | CTCCATCGAGAAAACCATCAGCAAGGCCAAGGGCCAGCCCCGCGAACCCCAGGTGTACAC |
| | | ACTGCCTCCAAGCCAGGAAGAGATGACCAAGAACCAGGTGTCCCTGACCTGTCTCGTGAA |
| | | AGGCTTCTACCCCTCCGATATCGCCGTGGAATGGGAGAGCAACGGCCAGCCCGAGAACAA |
| | | CTACAAGACCACCCCCCCTGTGCTGGACAGCGACGGCTCATTCTTCCTGTACAGCAGACTG |
| | | ACCGTGGACAAGAGCCGGTGGCAGGAAGGCAACGTGTTCAGCTGCAGCGTGATGCACGAG |
| | | GCCCTGCACAACCACTACACCCAGAAGTCCCTGTCTCTGTCCCTGGGCAAA |
| 69 | FLT3L ECD (Δ | ACCCAGGACTGTTCCTTCCAGCACTCCCCTATCTCCAGCGACTTCGCCGTGAAGATCAGAG |
| | C-term 5aa (PTAPQ; | AGCTGTCCGACTATCTGCTGCAGGACTACCCTGTGACCGTGGCCAGCAATCTGCAGGACGA |
| | SEQ ID NO: 85)) - | AGAACTGTGTGGTGGCCTGTGGCGACTGGTGTTGGCTCAGAGATGGATGGAACGGCTGAA |
| | linker SST/AAA - | AACCGTGGCCGGCTCTAAGATGCAGGGCCTGCTGGAAAGAGTGAACACCGAGATCCACTT |
| | hG4 | CGTGACCAAGTGCGCCTTTCAGCCTCCTCCATCCTGCCTGAGATTCGTGCAGACCAACATCT |
| | (S228P/F234A/ | CCCGGCTGCTGCAAGAGACATCCGAGCAGCTGGTGGCTCTGAAGCCCTGGATCACCCGGC |
| | L235A/M252Y/S254T/ | AGAACTTCTCTCGGTGTCTGGAACTGCAGTGTCAGCCTGATGCTGCCGCTTTGCCTCCACCT |
| | T256E) | TGGAGCCCTAGACCTCTGGAAGCTACCGCCGAGTCTAAGTACGGACCTCCTTGTCCTCCAT |
| | | GTCCTGCTCCAGAAGCTGCTGGCGGCCCTTCCGTGTTTCTGTTCCCTCCAAAGCCCAAGGAC |
| | | ACCCTGTACATCACCCGGGAACCCGAAGTGACCTGCGTGGTGGTGGATGTGTCCCAGGAA |
| | | GATCCCGAGGTGCAGTTCAATTGGTACGTGGACGGCGTGGAAGTGCACAACGCCAAGACC |
| | | AAGCCCAGAGAGGAACAGTTCAACAGCACCTACCGGGTGGTGTCCGTGCTGACAGTGCTG |
| | | CACCAGGACTGGCTGAACGGCAAAGAGTACAAGTGCAAGGTGTCCAACAAGGGCCTGCCC |
| | | AGCTCCATCGAGAAAACCATCAGCAAGGCCAAGGGCCAGCCCCGCGAACCCCAGGTGTAC |
| | | ACACTGCCTCCAAGCCAGGAAGAGATGACCAAGAACCAGGTGTCCCTGACCTGTCTCGTG |
| | | AAAGGCTTCTACCCCTCCGATATCGCCGTGGAATGGGAGAGCAACGGCCAGCCCGAGAAC |
| | | AACTACAAGACCACCCCCCCTGTGCTGGACAGCGACGGCTCATTCTTCCTGTACAGCAGAC |
| | | TGACCGTGGACAAGAGCCGGTGGCAGGAAGGCAACGTGTTCAGCTGCAGCGTGATGCACG |
| | | AGGCCCTGCACAACCACTACACCCAGAAGTCCCTGTCTCTGTCCCTGGGCAAA |
| 70 | FLT3L ECD (Δ | ACCCAGGACTGTTCCTTCCAGCACTCCCCTATCTCCAGCGACTTCGCCGTGAAGATCAGAG |
| | C-term 5aa (PTAPQ; | AGCTGTCCGACTATCTGCTGCAGGACTACCCTGTGACCGTGGCCAGCAATCTGCAGGACGA |
| | SEQ ID NO: 85))- | AGAACTGTGTGGTGGCCTGTGGCGACTGGTGTTGGCTCAGAGATGGATGGAACGGCTGAA |
| | linker SST/AAA; | AACCGTGGCCGGCTCTAAGATGCAGGGCCTGCTGGAAAGAGTGAACACCGAGATCCACTT |
| | S170A/S180A- | CGTGACCAAGTGCGCCTTTCAGCCTCCTCCATCCTGCCTGAGATTCGTGCAGACCAACATCT |
| | hG4 | CCCGGCTGCTGCAAGAGACATCCGAGCAGCTGGTGGCTCTGAAGCCCTGGATCACCCGGC |
| | (S228P/F234A/L23 | AGAACTTCTCTCGGTGTCTGGAACTGCAGTGTCAGCCTGATGCTGCCGCTTTGCCTCCTCCT |
| | 5A/M252Y/S254T/ | TGGGCTCCTCGACCTCTGGAAGCTACAGCCGAGGCTAAGTATGGCCCTCCTTGTCCTCCAT |
| | T256E) | GTCCTGCTCCAGAAGCTGCTGGCGGCCCTTCCGTGTTTCTGTTCCCTCCAAAGCCCAAGGAC |
| | | ACCCTGTACATCACCCGGGAACCCGAAGTGACCTGCGTGGTGGTGGATGTGTCCCAGGAA |
| | | GATCCCGAGGTGCAGTTCAATTGGTACGTGGACGGCGTGGAAGTGCACAACGCCAAGACC |
| | | AAGCCCAGAGAGGAACAGTTCAACAGCACCTACCGGGTGGTGTCCGTGCTGACAGTGCTG |
| | | CACCAGGACTGGCTGAACGGCAAAGAGTACAAGTGCAAGGTGTCCAACAAGGGCCTGCCC |
| | | AGCTCCATCGAGAAAACCATCAGCAAGGCCAAGGGCCAGCCCCGCGAACCCCAGGTGTAC |
| | | ACACTGCCTCCAAGCCAGGAAGAGATGACCAAGAACCAGGTGTCCCTGACCTGTCTCGTG |
| | | AAAGGCTTCTACCCCTCCGATATCGCCGTGGAATGGGAGAGCAACGGCCAGCCCGAGAAC |
| | | AACTACAAGACCACCCCCCCTGTGCTGGACAGCGACGGCTCATTCTTCCTGTACAGCAGAC |
| | | TGACCGTGGACAAGAGCCGGTGGCAGGAAGGCAACGTGTTCAGCTGCAGCGTGATGCACG |
| | | AGGCCCTGCACAACCACTACACCCAGAAGTCCCTGTCTCTGTCCCTGGGCAAA |

4. Vectors and Host Cells

Further provided are vectors comprising one or more polynucleotides encoding one or more of the FLT3L-Fc fusion proteins, described herein. A vector can be of any type, for example, a recombinant vector such as an expression vector. Vectors include without limitation, plasmids, cosmids, bacterial artificial chromosomes (BAC) and yeast artificial chromosomes (YAC) and vectors derived from bacteriophages or plant or animal (including human) viruses. Vectors can comprise an origin of replication recognized by the proposed host cell and in the case of expression vectors, promoter and other regulatory regions recognized by the host cell. In additional embodiments, a vector comprises a polynucleotide encoding an antibody of the disclosure operably linked to a promoter and optionally additional regulatory elements. Certain vectors are capable of autonomous replication in a host into which they are introduced (e.g., vectors having a bacterial origin of replication can replicate in bacteria). Other vectors can be integrated into the genome of a host upon introduction into the host, and thereby are replicated along with the host genome. Vectors include without limitation, those suitable for recombinant production of the antibodies disclosed herein.

The choice of the vector is dependent on the recombinant procedures followed and the host used. Introduction of vectors into host cells can be affected by inter alia calcium phosphate transfection, virus infection, DEAE-dextran-mediated transfection, lipofectamine transfection or electroporation. Vectors may be autonomously replicating or may replicate together with the chromosome into which they have been integrated. In certain embodiments, the vectors contain one or more selection markers. The choice of the markers may depend on the host cells of choice. These include without limitation, kanamycin, neomycin, puromycin, hygromycin, zeocin, thymidine kinase gene from Herpes simplex virus (HSV-TK), and dihydrofolate reductase gene from mouse (dhfr). Vectors comprising one or more nucleic acid molecules encoding the FLT3L-Fc fusion proteins described herein, operably linked to one or more nucleic acid molecules encoding proteins or peptides that can be used to isolate the FLT3L-Fc fusion proteins, are also covered by the disclosure. These proteins or peptides include without limitation, glutathione-S-transferase, maltose binding protein, metal-binding polyhistidine, green fluorescent protein, luciferase and beta-galactosidase.

In other embodiments, the vector that is used is pcDNA™3.1+(ThermoFisher, MA).

In some embodiments, the viral vector comprises an oncolytic viral vector. As appropriate, the oncolytic viral vector can be a DNA virus or a RNA virus. In some embodiments, the viral vector is from a viral family selected from the group consisting of: Adenoviridae (e.g., Adenovirus), Arenaviridae (e.g., lymphocytic choriomeningitis mammarenavirus, Cali mammarenavirus (a.k.a., Pichinde mammarenavirus), Poxviridae (e.g., Vaccinia virus), Herpesviridae (e.g., Herpesvirus, e.g., HSV-1), Parvoviridae (e.g., Parvovirus H1), Reoviridae (e.g., Reovirus), Picornaviridae (e.g., Coxsackievirus, Seneca Valley Virus, Poliovirus), Paramyxoviridae (e.g., Measles virus, Newcastle disease virus (NDV)), Rhabdoviridae (e.g., Vesicular stomatitis virus (VSV)), Togaviridae (e.g., Alphavirus, Sindbis virus), Enteroviridae (e.g., Echovirus). The use of oncolytic viruses in cancer therapy is described e.g., Fukuhara, et al., Cancer Sci. (2016) 107(10):1373-1379; Kaufman, et al., Nat Rev Drug Discov. (2015) 14(9):642-62; Hamid, et al., Cancer Immunol Immunother. (2017) 66(10):1249-1264; Taguchi, et al., Int J Urol. (2017) 24(5):342-351; and Buijs, et al., Hum Vaccin Immunother. (2015) 11(7):1573-84.

The disclosure also provides host cells comprising a nucleic acid or a vector described herein. Any of a variety of host cells can be used. In one embodiment, a host cell is a prokaryotic cell, for example, E. coli. In another embodiment, a host cell is a eukaryotic cell, for example, a yeast cell, a plant cell, an insect cell, a mammalian cell, such as a Chinese Hamster Ovary (CHO)-based or CHO-origin cell line (e.g., CHO-S, CHO DG44, ExpiCHO™, CHOZN® ZFN-modified GS−/− CHO cell line, CHO-K1, CHO-K1a), COS cells, BHK cells, NSO cells or Bowes melanoma cells. Examples of human host cells are, inter alia, HeLa, 911, AT1080, A549 and HEK293 (e.g., HEK293E, HEK293T, Expi293™). In addition, the FLT3L-Fc fusion proteins can be expressed in a yeast cell such as Pichia (see, e.g., Powers et al., J Immunol Methods. 251:123-35 (2001)), Hanseula, or Saccharomyces.

In some embodiments, the host cell predominantly sialylates N-linked glycosylation sites of the fusion protein. In some embodiments, the polynucleotides encoding a FLT3L-Fc fusion protein, as described herein, are expressed in a host cell that sialylates at least 50%, at least 55%, at least 60%, at least 65%, at least 70%, at least 75%, at least 80%, at least 85%, at least 90%, at least 91%, at least 92%, at least 93%, at least 94%, at least 95%, or more, N-linked glycosylation sites in the expressed FLT3L-Fc fusion proteins.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Some vectors are suitable for delivering the nucleic acid molecule or polynucleotide of the present application. Certain vectors are capable of directing the expression of nucleic acids to which they are operatively linked. Such vectors are referred to herein as expression vectors.

The term “operably linked” refers to two or more nucleic acid sequence or polypeptide elements that are usually physically linked and are in a functional relationship with each other. For instance, a promoter is operably linked to a coding sequence if the promoter is able to initiate or regulate the transcription or expression of a coding sequence, in which case, the coding sequence should be understood as being “under the control of” the promoter.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As appropriate, the host cells can be stably or transiently transfected with a polynucleotide encoding a FLT3L-Fc fusion protein, as described herein.

5. Methods of Producing FLT3L-Fc Fusion Proteins

The FLT3L-Fc fusion proteins described herein can be produced by any method known in the art for the synthesis of fusion proteins, for example, by chemical synthesis or by recombinant expression techniques.

Methods of recombinant expression of fusion proteins are known and can be applied to the recombinant production and isolation/purification of the FLT3L-Fc fusion proteins. Methods of recombinantly expressing proteins, including fusion proteins, are described, for example, in Green and Sambrook, “Molecular Cloning: A Laboratory Manual,” 4^{th} Edition, 2012, Cold Spring Harbor Laboratory Press; Current Protocols in Protein Science, Wiley, 1995-2109 (currentprotocols.onlinelibrary.wiley.com/journal/19343663/); and Current Protocols in Molecular Biology, Wiley, 1987-2019 (currentprotocols.onlinelibrary.wiley.com/journal/19343647/). In addition, other publications relating to producing recombinantly expressed fusion proteins include, e.g., Argelia Lorence (Editor), “Recombinant Gene Expression” (Methods in Molecular Biology) 2012, Humana Press; James L Hartley (Editor), “Protein Expression in Mammalian Cells: Methods and Protocols” (Methods in Molecular Biology) 2012, Humana Press; Roslyn M. Bill (Editor), “Recombinant Protein Production in Yeast: Methods and Protocols” (Methods in Molecular Biology) 2012, Humana Press; and MacDonald, Kolotilin and Menassa (Editors) “Recombinant Proteins from Plants: Methods and Protocols” (Methods in Molecular Biology), 2^{nd} Edition, 2016, Humana Press.

In various embodiments, the FLT3L-Fc fusion proteins described herein may be produced in bacterial or eukaryotic cells. The FLT3L-Fc fusion proteins can also be produced in eukaryotic cells such as transformed cell lines (e.g., CHO, CHO-S, CHO DG44, ExpiCHO™, CHOZN®, CHO-K1, CHO-K1a, 293E, 293T, COS, NIH3T3). In addition, the FLT3L-Fc fusion proteins described herein can be expressed in a yeast cell such as Pichia (see, e.g., Powers et al., J Immunol Methods. 251:123-35 (2001)), Hanseula, or Saccharomyces. In one embodiment, the FLT3L-Fc fusion proteins described herein are produced in a CHO cell line, e.g., a CHO-S, CHO DG44, ExpiCHO™, CHOZN®, CHO-K1, CHO-K1a cell line, or a HEK293 (e.g., HEK293E, HEK293T, Expi293™) cell line. To produce the FLT3L-Fc fusion proteins of interest (including a heterodimer comprising the FLT3-Fc fusion protein), one or more polynucleotides encoding the FLT3L-Fc fusion proteins is constructed, introduced into an expression vector, and then expressed in one or more suitable host cells. In some embodiments, three polynucleotides encoding a FLT3L-Fc fusion, a Fab heavy chain and a Fab light chain comprising a second antigen binding domain are co-expressed in a single host cell. Standard molecular biology techniques are used to prepare the recombinant expression vector, transfect the host cells, select for transformants, culture the host cells, and recover the FLT3L-Fc fusion proteins.

In some embodiments, the host cell predominantly sialylates N-linked glycosylation sites of the fusion protein. In some embodiments, the polynucleotides encoding a FLT3L-Fc fusion protein, as described herein, are expressed in a host cell that sialylates at least 50%, at least 55%, at least 60%, at least 65%, at least 70%, at least 75%, at least 80%, at least 85%, at least 90%, at least 91%, at least 92%, at least 93%, at least 94%, at least 95%, or more, N-linked glycosylation sites in the expressed FLT3L-Fc fusion proteins.

If the FLT3L-Fc fusion proteins are to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for secretion of the FLT3L-Fc fusion proteins. For production into the periplasm of E. coli, the pelB signal sequence (Lei et al., J. Bacteriol., 169: 4379 (1987)) may be used as the signal sequence for secretion of the FLT3L-Fc fusion proteins. For bacterial expression, calcium chloride methods or electroporation methods may be used to introduce the expression vector into the bacterial cell.

If the FLT3L-Fc fusion proteins are to be expressed in mammalian cells (e.g., such as CHO-S, CHO DG44, ExpiCHO™, CHOZN®, CHO-K1, CHO-K1a, 293E, 293T, Expi293™, COS, NIH3T3 cells), the expression vector includes a promoter to promote expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, the FLT3L-Fc fusion proteins are produced in mammalian cells. Exemplary mammalian host cells for expressing FLT3L-Fc fusion proteins include Chinese Hamster Ovary (e.g., CHO, CHO-S, CHO DG44, ExpiCHO™, CHOZN®, CHO-K1, CHO-K1a) (including dhfr− CHO cells, described in Urlaub and Chasin (1980) Proc. Natl. Acad. Sci. USA 77: 4216-4220, used with a DHFR selectable marker, e.g., as described in Kaufman and Sharp (1982) Mol. Biol. 159:601 621), human embryonic kidney 293 cells (e.g., 293, 293E, 293T, Expi293™), COS cells, NIH3T3 cells, lymphocytic cell lines, e.g., NSO myeloma cells and SP2 cells, and a cell from a transgenic animal, e.g., a transgenic mammal. For example, in some embodiments, the cell is a mammary epithelial cell.

In an exemplary system for expression of the FLT3L-Fc fusion proteins, recombinant expression vectors encoding the FLT3L-Fc fusion protein are introduced into dhfr− CHO cells by calcium phosphate-mediated transfection. In a specific embodiment, the dhfr− CHO cells are cells of the DG44 cell line, such as DG44i (see, e.g., Derouaz et al., Biochem Biophys Res Commun., 340(4):1069-77 (2006)). Within the recombinant expression vectors, the polynucleotide encoding the FLT3L-Fc fusion protein, and optionally a second polynucleotide encoding a second Fc fusion protein for forming a heterodimer, are operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vectors also carry a DHFR gene, which allows for selection of CHO cells that have been transfected with the vector using methotrexate selection/amplification. The selected transformant host cells are cultured to allow for expression and secretion of the FLT3L-Fc fusion protein, and the fusion protein is recovered from the culture medium.

The FLT3L-Fc fusion proteins can also be produced by a transgenic animal. For example, U.S. Pat. No. 5,849,992 describes a method of expressing an antibody in the mammary gland of a transgenic mammal. A transgene is constructed that includes a milk-specific promoter and one or more polynucleotides encoding the FLT3L-Fc fusion protein of interest and a signal sequence for secretion. The milk produced by females of such transgenic mammals includes, secreted-therein, the FLT3L-Fc fusion protein of interest. The FLT3L-Fc fusion protein can be purified from the milk, or for some applications, used directly. Animals are also provided comprising one or more of the FLT3L-Fc-encoding nucleic acids described herein.

The FLT3L-Fc fusion proteins can be isolated from inside or outside (such as medium) of the host cell and purified as substantially pure and homogenous, non-aggregated FLT3L-Fc fusion proteins (e.g., including monomeric, homodimeric and/or heterodimeric bispecific FLT3L-Fc fusion proteins). Methods for isolation and purification commonly used for protein purification, including antibody purification, may be used for the isolation and purification of herein described FLT3L-Fc fusion proteins, and are not limited to any particular method. Applicable protein purification techniques are described, e.g., in Labrou, Chronopoulou and Ataya (Editors), “Handbook on Protein Purification: Industry Challenges and Technological Developments, 2018, Nova Science Pub Inc; Gottschalk (Editor), “Process Scale Purification of Antibodies,” 2nd Edition, 2017, Wiley; Staby, Rathore and Ahuja (Editors), “Preparative Chromatography for Separation of Proteins, 2017, Wiley; and Labrou (Editor), “Protein Downstream Processing: Design, Development and Application of High and Low-Resolution Methods, 2014, Human Press. The FLT3L-Fc fusion proteins may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes FLT3L-Fc fusion proteins that are highly purified using these purification methods.

6. Pharmaceutical Compositions

Provided are pharmaceutical compositions comprising a FLT3L-Fc fusion protein, as described herein, or a polynucleotide encoding a FLT3L-Fc fusion protein, as described herein, and a pharmaceutically acceptable diluent, carrier or excipient. In certain embodiments, the pharmaceutical composition comprises a therapeutically effective amount of the FLT3L-Fc fusion protein, or polynucleotide encoding such FLT3L-Fc fusion protein.

Various pharmaceutically acceptable diluents, carriers, and excipients, and techniques for the preparation and use of pharmaceutical compositions will be known to those of skill in the art in light of the present disclosure. Illustrative pharmaceutical compositions and pharmaceutically acceptable diluents, carriers, and excipients are also described in, e.g., Loyd V. Allen Jr (Editor), “Remington: The Science and Practice of Pharmacy,” 22^{nd} Edition, 2012, Pharmaceutical Press; Brunton, Knollman and Hilal-Dandan, “Goodman and Gilman's The Pharmacological Basis of Therapeutics,” 13th Edition, 2017, McGraw-Hill Education/Medical; McNally and Hastedt (Editors), “Protein Formulation and Delivery,” 2nd Edition, 2007, CRC Press; Banga, “Therapeutic Peptides and Proteins: Formulation, Processing, and Delivery Systems,” 3rd Edition, 2015, CRC Press; Lars Hovgaard, Frokjaer and van de Weert (Editors), “Pharmaceutical Formulation Development of Peptides and Proteins,” 2nd Edition, 2012, CRC Press; Carpenter and Manning (Editors), “Rational Design of Stable Protein Formulations: Theory and Practice,” 2002, Springer (Pharmaceutical Biotechnology (Book 13)); Meyer (Editor), “Therapeutic Protein Drug Products: Practical Approaches to Formulation in the Laboratory, Manufacturing, and the Clinic,” 2012, Woodhead Publishing; and Shire, “Monoclonal Antibodies: Meeting the Challenges in Manufacturing, Formulation, Delivery and Stability of Final Drug Product,” 2015, Woodhead Publishing.

In some embodiments, each carrier, diluent or excipient is “acceptable” in the sense of being compatible with the other ingredients of the pharmaceutical composition and not injurious to the subject. Often, the pharmaceutically acceptable carrier is an aqueous pH-buffered solution. Some examples of materials which can serve as pharmaceutically-acceptable carriers, diluents or excipients include: water; buffers, e.g., phosphate-buffered saline; sugars, such as lactose, trehalose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; amino acids (e.g., charged amino acids, including without limitation, aspartate, asparagine, glutamate, glutamine, histidine, lysine); and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

The formulation of and delivery methods of pharmaceutical compositions will generally be adapted according to the site and the disease to be treated. Exemplary formulations include without limitation, those suitable for parenteral administration, e.g., intratumoral, intravenous, intra-arterial, intramuscular, or subcutaneous administration, including formulations encapsulated in micelles, liposomes or drug-release capsules (active agents incorporated within a biocompatible coating designed for slow-release); ingestible formulations; formulations for topical use, such as creams, ointments and gels; and other formulations such as inhalants, aerosols and sprays. In some embodiments, the pharmaceutical compositions are formulated for parenteral, e.g., intravenous, subcutaneous, or oral administration. In some embodiments, the pharmaceutical compositions are formulated for intratumoral administration.

In certain embodiments, pharmaceutical compositions are sterile. In certain embodiments, the pharmaceutical composition has a pH in the range of 4.5 to 8.5, 4.5 to 6.5, 6.5 to 8.5, or a pH of about 5.0, about 5.5, about 6.0, about 6.5, about 7.0, about 7.5, about 8.0 or about 8.5. In some embodiments, the pharmaceutical composition has a pH of 5.9. In one embodiment, the pharmaceutical composition has an osmolarity in the range of 240-260 or 250-330 mOsmol/L. In certain embodiments, the pharmaceutical composition is isotonic or near isotonic.

In some embodiments, the pharmaceutical compositions are liquids or solids. In some embodiments, the pharmaceutical composition comprises an aqueous solution, e.g., at a concentration in the range of about 1 mg/ml to about 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml or 20 mg/ml. In some embodiments, the pharmaceutical composition comprises an aqueous solution of FLT3L-Fc fusion protein at a concentration of about 2 mg/ml. In some embodiments, the pharmaceutical composition comprises an aqueous solution of FLT3L-Fc fusion protein at a concentration of 2 mg/ml. In some embodiments, the pharmaceutical composition is lyophilized. In certain embodiments, the pharmaceutical composition is formulated for intravenous administration and has a concentration of FLT3L-Fc fusion protein of about 1-100 mg/ml, 1-10 mg/ml, 2-20 mg/ml or about 1, 2, 3, 4, 5, 6, 7, 8, 9 or 10 mg/ml. In certain embodiments, the pharmaceutical composition is formulated for intravenous administration and has a concentration of FLT3L-Fc fusion protein of about 2 mg/ml. In certain embodiments, the pharmaceutical composition is formulated for intravenous administration and has a concentration of FLT3L-Fc fusion protein of 2 mg/ml. In some embodiments, the pharmaceutical composition is formulated for subcutaneous injection and has a concentration of FLT3L-Fc fusion protein of 1-100 mg/ml, 1-10 mg/ml, 2-20 mg/ml or about 1, 2, 3, 4, 5, 6, 7, 8, 9 or 10 mg/ml, and a viscosity less than 50 cP, less than 30 cP, less than 20 cP, or about 10 cP.

In some embodiments, the pharmaceutical composition is an aqueous solution containing 2 mg/mL FLT3L-Fc fusion protein in 20 mM histidine, 90 g/L sucrose, 0.2 g/L polysorbate 80, pH 5.9.

In some embodiments, the pharmaceutical composition further comprises one or more additional therapeutic agents, e.g., a second therapeutic agent, or second and third therapeutic agents.

7. Methods of Treatment

Provided are methods of preventing, reducing and/or inhibiting the recurrence, growth, proliferation, migration and/or metastasis of a cancer cell or population of cancer cells in a subject in need thereof, comprising administering to the subject an effective amount of fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. Alternatively, in some embodiments, the method of preventing, reducing and/or inhibiting the recurrence, growth, proliferation, migration and/or metastasis of a cancer cell or population of cancer cells in a subject in need thereof, comprises administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan. Alternatively, in some embodiments, the method of preventing, reducing and/or inhibiting the recurrence, growth, proliferation, migration and/or metastasis of a cancer cell or population of cancer cells in a subject in need thereof, comprising administering to the subject (I) an effective amount of a human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor. Alternatively, in some embodiments, the method of preventing, reducing and/or inhibiting the recurrence, growth, proliferation, migration and/or metastasis of a cancer cell or population of cancer cells in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor. In some embodiments, the FLT3L modulator is a fusion protein comprising a FLT3L protein or fragment thereof and an Fc protein or fragment thereof. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18, 21-27, 114, and 115. In some embodiments, the Fc protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 111. In some embodiments, residues 13-17 of SEQ ID NO: 111 comprise the amino acid sequence PVAGT (SEQ ID NO: 116) and residue 76 of SEQ ID NO: 111 is a glycine. In some embodiments, the FLT3L protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NOs: 112, 113, or 117. In some embodiments, the FLT3L protein or fragment thereof comprises CDX-301, which is disclosed in International Publication No. 94/28391. In some embodiments, the FLT3L modulator comprises a FLT3L protein, Fc protein, or FLT3L-Fc fusion protein, or a fragment thereof as disclosed in International Publication No. 2022/031876. In some embodiments, the immunoconjugate is co-administered with the FLT3L modulator. In some embodiments, the FLT3L modulator comprises the amino acid sequence of any one of SEQ ID NOs: 101-105 and 107. In some embodiments, the immunoconjugate comprises datopotamab deruxtecan (DS-1062). In some embodiments, the FLT3L modulator comprises the amino acid sequence of any one of SEQ ID NOs: 111-115 and 117 or comprises CDX-301 and the immunoconjugate comprises datopotamab deruxtecan (DS-1062). In some embodiments, the effective amount of the fusion protein is at least about 200 μg, 225 μg, 300 μg, 500 μg, 600 μg, 675 μg, 700 μg, 800 μg, 900 μg, 1000 μg, 1200 μg, 1400 μg, 1600 μg, 1800 μg, or 2000 μg of the fusion protein per dose. In some embodiments, the effective amount of the fusion protein is between about 200 μg to about 30000 μg, about 200 μg to about 25000 μg, about 200 μg to about 20000 μg, about 500 μg to about 20000 μg, about 500 μg to about 15000 μg, about 500 μg to about 10000 μg, about 600 μg to about 20000 μg, about 600 μg to about 15000 μg, about 600 μg to about 10000 μg, about 600 μg to about 8000 μg, about 600 μg to about 5000 μg, about 600 μg to about 3000 μg, about 600 μg to about 2500 μg, about 600 μg to about 2000 μg of the fusion protein per dose. In some embodiments, the effective amount of the fusion protein is between about 200 μg to about to about 2500 μg of the fusion protein per dose. In some embodiments, the effective amount of the fusion protein is between about 600 μg to about 2000 μg of the fusion protein per dose. In some embodiments, the effective amount of the fusion protein is less than about 30000 μg, 25000 μg, 20000 μg, 15000 μg, 10000 μg, 9000 μg, 8000 μg, 7000 μg, 6000 μg, 5000 μg, 4000 μg, 3000 μg, or 2000 μg of the fusion protein per dose. In some embodiments, the effective amount of the fusion protein is less than about 25000 μg of the fusion protein per dose. In some embodiments, the effective amount of the fusion protein is less than about 20000 μg of the fusion protein per dose. In some embodiments, the effective amount of the fusion protein is less than about 15000 μg of the fusion protein per dose. In some embodiments, the effective amount of the fusion protein is less than about 10000 μg of the fusion protein per dose. In some embodiments, the effective amount of the fusion protein is less than about 5000 μg of the fusion protein per dose. In some embodiments, at least two doses of the fusion protein are administered at least two weeks apart. In some embodiments, at least two doses are administered 2 to 4 weeks apart. In some embodiments, at least two doses are administered at least about 2 weeks apart over a duration of at least about 1 month. In some embodiments, at least two doses are administered between about 2 to 5 weeks apart over a duration of between about 1 to 4 months. In some embodiments, between about 3 to about 8 doses of the fusion protein. In some embodiments, between about 3 to about 8 doses of the fusion protein, wherein at least two of the doses are administered between 2 to about 5 weeks apart over a duration of between 1 to 4 months. In some embodiments, the method further comprises pausing administration of the fusion protein for a period of at least about 6, 7, 8, 9, 10, 11, 12, 13, 14, 15, 16, 17, 18, 19, or 20 weeks. In some embodiments, the method further comprises pausing administration of the fusion protein for a period of up to about 3, 4, 5, 6, 7, 8, 9, 10, 11, 12, 13, 14, 15, 16, 17, 18, 19, or 20 months. In some embodiments, the method further comprises pausing administration of the fusion protein for a period of between about 6 weeks to about 8 months, about 2 to about 8 months, about 3 to about 8 months, about 2 to about 10 months, about 2 to about 6 months, about 3 to about 6 months, about 4 to about 8 months, or about 4 to about 6 months. In some embodiments, administration of the fusion protein is paused for at least about 6 weeks. In some embodiments, administration of the fusion protein is paused for at least about 10 weeks. In some embodiments, administration of the fusion protein is paused for at least about 12 weeks. In some embodiments, administration of the fusion protein is paused for at least about 16 weeks. In some embodiments, administration of the fusion protein is paused for up to about 10 months. In some embodiments, administration of the fusion protein is paused for up to about 9 months. In some embodiments, administration of the fusion protein is paused for up to about 8 months. In some embodiments, administration of the fusion protein is paused for up to about 7 months. In some embodiments, administration of the fusion protein is paused for up to about 6 months. In some embodiments, the method further comprises co-administering to the subject an anti-cancer agent. In some embodiments, the anti-cancer agent is sacituzumab govitecan. In some embodiments, the anti-cancer agent is an anti-CD47 antibody. In some embodiments, the anti-cancer agent is magrolimab. In some embodiments, the anti-cancer agent is an inhibitor of MCL-1. In some embodiments, the immunoconjugate is co-administered with the fusion protein. In some embodiments, the immunconjugate comprises an anti-Trop2-ADC. In some embodiments, the immunoconjugate comprises an anti-Trop2 antibody. In some embodiments, a FLT3R agonist is co-administered with the fusion protein. In some embodiments, the FLT3R agonist is selected from an antibody, small molecule, or cytokine. In some embodiments, an anti-PD1 antibody is co-administered with the fusion protein. In some embodiments, the anti-PD1 antibody is selected from balstilimab, budigalimab, camrelizumab, cemiplimab, cetrelimab, dostarlimab, genolimzumab, nivolumab, pembrolizumab, pidilizumab, prolgolimab, retifanlimab, sasanlimab, sintilimab, spartalizumab, tislelizumab, toripalimab, and zimberelimab. In some embodiments, the anti-PD1 antibody is zimberelimab. In some embodiments, an anti-PDL1 antibody is co-administered with the fusion protein. In some embodiments, the anti-PDL1 antibody is selected from atezolizumab, avelumab, cosibelimab, durvalumab, envafolimab, and lodapolimab. In some embodiments, the anti-PDL1 antibody is atezolizumab. In some embodiments, an anti-Tigit antibody is co-administered with the fusion protein. In some embodiments, the anti-Tigit antibody is selected from AB-308, AGEN-1307 (AGEN-1327), AGEN-1777, AK127, BMS-986207, domvanalimab, EOS-448, etigilimab, JS006, ociperlimab, SEA-TGT (SGN-TGT), tiragolumab, and vibostolimab. In some embodiments, the anti-Tigit antibody is vibostolimab. In some embodiments, the anti-Tigit antibody is AB-308. In some embodiments, the anti-Tigit antibody is domvanalimab. In some embodiments, MCL-1 inhibitor is co-administered with the fusion protein. In some embodiments, the MCL-1 inhibitor is selected from GS-9716, 564315 (MIK665), AZD5991, AMG-176, AMG-397, ABBV-467, and PRT1419. In some embodiments, the anti-CD47 antibody is co-administered with the fusion protein. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments, the adenosine pathway inhibitor is co-administered with the fusion protein. In some embodiments, the adenosine pathway inhibitor is selected from an adenosine receptor antagonist, CD39 inhibitor, and CD73 inhibitor. In some embodiments, the adenosine receptor antagonist is a small molecule. In some embodiments, the adenosine receptor antagonist is selected from etrumadenant (AB729; GS-0928), M1069, taminadenant, TT-4, and TT-10. In some embodiments, the adenosine receptor antagonist is AB729 (etrumadenant). In some embodiments, the CD39 inhibitor is selected from TTX-030, IPH5201, SRF617, nucleotide derivatives, anthraquinone derivatives, and suramin derivatives. In some embodiments, the anthraquinone derivate is RB2. In some embodiments, the CD73 inhibitor is a small molecule. In some embodiments, CD73 inhibitor is selected from AB680 (quemliclustat), AK131, ATG-037, BMS-986179, mupadolimab, NZV930, oleclumab, ORIC-533, PT-199, and uliledlimab. In some embodiments, the CD73 inhibitor is AB680 (quemliclustat). In some embodiments, the anti-CCR8 antibody is co-administered with the fusion protein. In some embodiments, anti-CCR8 antibody causes depletion of regulatory T (Treg) cells. In some embodiments, the anti-CCR8 antibody is selected from BMS-986340, FPA157, HFB1011, HBM1022, IO-1, IPG276, JTX-1811 (GS-1811), LM-108, S-531011, and SRF-114. In some embodiments, the anti-CCR8 antibody is JTX-1811 (GS-1811). In some embodiments, the method further comprises co-administering to the subject an immunotherapy. In some embodiments, the method further comprises co-administering to the subject an immune checkpoint protein or receptor. In some embodiments, the method further comprises co-administering to the subject an inhibitor of an immune checkpoint protein or receptor. In some embodiments, the method further comprises co-administering to the subject an activator of an immune checkpoint protein or receptor. In some embodiments, the subject is a human subject. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 97% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 98% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 99% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence selected from the group consisting of SEQ ID NOs: 1-18 and 21-27. In some embodiments, the fusion protein comprises an amino acid sequence that is at least about 97% identical to the amino acid sequence of SEQ ID NO: 14. In some embodiments, the fusion protein comprises an amino acid sequence that is at least about 98% identical to the amino acid sequence of SEQ ID NO: 14. In some embodiments, the fusion protein comprises an amino acid sequence that is at least about 99% identical to the amino acid sequence of SEQ ID NO: 14. In some embodiments, the fusion protein comprises the amino acid sequence of SEQ ID NO: 14.

Provided are methods of treating and/or inhibiting cancer in a subject in need thereof, comprising administering to the subject an effective amount of fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. Alternatively, in some embodiments, the method of treating and/or inhibiting cancer in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan. Alternatively, in some embodiments, the method of treating and/or inhibiting in a subject in need thereof, comprising administering to the subject (I) an effective amount of a human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor. Alternatively, in some embodiments, the method of treating and/or inhibiting in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor. In some embodiments, the FLT3L modulator is a fusion protein comprising a FLT3L protein or fragment thereof and an Fc protein or fragment thereof. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18, 21-27, 114, and 115. In some embodiments, the Fc protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 111. In some embodiments, residues 13-17 of SEQ ID NO: 111 comprise the amino acid sequence PVAGT (SEQ ID NO: 161) and residue 76 of SEQ ID NO: 111 is a glycine. In some embodiments, the FLT3L protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NOs: 112, 113, or 117. In some embodiments, the FLT3L protein or fragment thereof comprises CDX-301, which is disclosed in International Publication No. 94/28391. In some embodiments, the FLT3L modulator comprises a FLT3L protein, Fc protein, or FLT3L-Fc fusion protein, or a fragment thereof as disclosed in International Publication No. 2022/031876. In some embodiments, the immunoconjugate is co-administered with the FLT3L modulator. In some embodiments, the FLT3L modulator comprises the amino acid sequence of any one of SEQ ID NOs: 111-115 and 117. In some embodiments, the immunoconjugate comprises datopotamab deruxtecan (DS-1062). In some embodiments, the FLT3L modulator comprises the amino acid sequence of any one of SEQ ID NOs: 111-115 and 117 or comprises CDX-301 and the immunoconjugate comprises datopotamab deruxtecan (DS-1062). In some embodiments, the effective amount of the fusion protein is at least about 200 μg, 225 μg, 300 μg, 500 μg, 600 μg, 675 μg, 700 μg, 800 μg, 900 μg, 1000 μg, 1200 μg, 1400 μg, 1600 μg, 1800 μg, or 2000 μg of the fusion protein per dose. In some embodiments, the effective amount of the fusion protein is between about 200 μg to about 30000 μg, about 200 μg to about 25000 μg, about 200 μg to about 20000 μg, about 500 μg to about 20000 μg, about 500 μg to about 15000 μg, about 500 μg to about 10000 μg, about 600 μg to about 20000 μg, about 600 μg to about 15000 μg, about 600 μg to about 10000 μg, about 600 μg to about 8000 μg, about 600 μg to about 5000 μg, about 600 μg to about 3000 μg, about 600 μg to about 2500 μg, about 600 μg to about 2000 μg of the fusion protein per dose. In some embodiments, the effective amount of the fusion protein is between about 200 μg to about to about 2500 μg of the fusion protein per dose. In some embodiments, the effective amount of the fusion protein is between about 600 μg to about 2000 μg of the fusion protein per dose. In some embodiments, the effective amount of the fusion protein is less than about 30000 μg, 25000 μg, 20000 μg, 15000 μg, 10000 μg, 9000 μg, 8000 μg, 7000 μg, 6000 μg, 5000 μg, 4000 μg, 3000 μg, or 2000 μg of the fusion protein per dose. In some embodiments, the effective amount of the fusion protein is less than about 25000 μg of the fusion protein per dose. In some embodiments, the effective amount of the fusion protein is less than about 20000 μg of the fusion protein per dose. In some embodiments, the effective amount of the fusion protein is less than about 15000 μg of the fusion protein per dose. In some embodiments, the effective amount of the fusion protein is less than about 10000 μg of the fusion protein per dose. In some embodiments, the effective amount of the fusion protein is less than about 5000 μg of the fusion protein per dose. In some embodiments, at least two doses of the fusion protein are administered at least two weeks apart. In some embodiments, at least two doses are administered 2 to 4 weeks apart. In some embodiments, at least two doses are administered at least about 2 weeks apart over a duration of at least about 1 month. In some embodiments, at least two doses are administered between about 2 to 5 weeks apart over a duration of between about 1 to 4 months. In some embodiments, between about 3 to about 8 doses of the fusion protein. In some embodiments, between about 3 to about 8 doses of the fusion protein, wherein at least two of the doses are administered between 2 to about 5 weeks apart over a duration of between 1 to 4 months. In some embodiments, the method further comprises pausing administration of the fusion protein for a period of at least about 6, 7, 8, 9, 10, 11, 12, 13, 14, 15, 16, 17, 18, 19, or 20 weeks. In some embodiments, the method further comprises pausing administration of the fusion protein for a period of up to about 3, 4, 5, 6, 7, 8, 9, 10, 11, 12, 13, 14, 15, 16, 17, 18, 19, or 20 months. In some embodiments, the method further comprises pausing administration of the fusion protein for a period of between about 6 weeks to about 8 months, about 2 to about 8 months, about 3 to about 8 months, about 2 to about 10 months, about 2 to about 6 months, about 3 to about 6 months, about 4 to about 8 months, or about 4 to about 6 months. In some embodiments, administration of the fusion protein is paused for at least about 6 weeks. In some embodiments, administration of the fusion protein is paused for at least about 10 weeks. In some embodiments, administration of the fusion protein is paused for at least about 12 weeks. In some embodiments, administration of the fusion protein is paused for at least about 16 weeks. In some embodiments, administration of the fusion protein is paused for up to about 10 months. In some embodiments, administration of the fusion protein is paused for up to about 9 months. In some embodiments, administration of the fusion protein is paused for up to about 8 months. In some embodiments, administration of the fusion protein is paused for up to about 7 months. In some embodiments, administration of the fusion protein is paused for up to about 6 months. In some embodiments, the method further comprises co-administering to the subject an anti-cancer agent. In some embodiments, the method further comprises co-administering to the subject an immunotherapy. In some embodiments, the method further comprises co-administering to the subject an immune checkpoint protein or receptor. In some embodiments, the method further comprises co-administering to the subject an inhibitor of an immune checkpoint protein or receptor. In some embodiments, the method further comprises co-administering to the subject an activator of an immune checkpoint protein or receptor. In some embodiments, the method further comprises co-administering to the subject sacituzumab govitecan. In some embodiments, the method further comprises co-administering to the subject an anti-CD47 antibody. In some embodiments, the method further comprises co-administering to the subject magrolimab. In some embodiments, the method further comprises co-administering to the subject an inhibitor of MCL-1. In some embodiments, the immunoconjugate is co-administered with the fusion protein. In some embodiments, the immunconjugate comprises an anti-Trop2-ADC. In some embodiments, the immunoconjugate comprises an anti-Trop2 antibody. In some embodiments, the FLT3R agonist is co-administered with the fusion protein. In some embodiments, the FLT3R agonist is selected from an antibody, small molecule, or cytokine. In some embodiments, the anti-PD1 antibody is co-administered with the fusion protein. In some embodiments, the anti-PD1 antibody is selected from balstilimab, budigalimab, camrelizumab, cemiplimab, cetrelimab, dostarlimab, genolimzumab, nivolumab, pembrolizumab, pidilizumab, prolgolimab, retifanlimab, sasanlimab, sintilimab, spartalizumab, tislelizumab, toripalimab, and zimberelimab. In some embodiments, the anti-PD1 antibody is zimberelimab. In some embodiments, an anti-PDL1 antibody is co-administered with the fusion protein. In some embodiments, the anti-PDL1 antibody is selected from atezolizumab, avelumab, cosibelimab, durvalumab, envafolimab, and lodapolimab. In some embodiments, the anti-PDL1 antibody is atezolizumab. In some embodiments, the anti-Tigit antibody is co-administered with the fusion protein. In some embodiments, the anti-Tigit antibody is selected from AB-308, AGEN-1307 (AGEN-1327), AGEN-1777, AK127, BMS-986207, domvanalimab, EOS-448, etigilimab, JS006, ociperlimab, SEA-TGT (SGN-TGT), tiragolumab, and vibostolimab. In some embodiments, the anti-Tigit antibody is vibostolimab. In some embodiments, the anti-Tigit antibody is AB-308. In some embodiments, the anti-Tigit antibody is domvanalimab. In some embodiments, MCL-1 inhibitor is co-administered with the fusion protein. In some embodiments, the MCL-1 inhibitor is selected from GS-9716, 564315 (MIK665), AZD5991, AMG-176, AMG-397, ABBV-467, and PRT1419. In some embodiments, the anti-CD47 antibody is co-administered with the fusion protein. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments, the adenosine pathway inhibitor is co-administered with the fusion protein. In some embodiments, the adenosine pathway inhibitor is selected from an adenosine receptor antagonist, CD39 inhibitor, and CD73 inhibitor. In some embodiments, the adenosine receptor antagonist is a small molecule. In some embodiments, the adenosine receptor antagonist is selected from etrumadenant (AB729; GS-0928), M1069, taminadenant, TT-4, and TT-10. In some embodiments, the adenosine receptor antagonist is AB729 (etrumadenant). In some embodiments, the CD39 inhibitor is selected from TTX-030, IPH5201, SRF617, nucleotide derivatives, anthraquinone derivatives, and suramin derivatives. In some embodiments, the anthraquinone derivate is RB2. In some embodiments, the CD73 inhibitor is a small molecule. In some embodiments, CD73 inhibitor is selected from AB680 (quemliclustat), AK131, ATG-037, BMS-986179, mupadolimab, NZV930, oleclumab, ORIC-533, PT-199, and uliledlimab. In some embodiments, the CD73 inhibitor is AB680 (quemliclustat). In some embodiments, the anti-CCR8 antibody is co-administered with the fusion protein. In some embodiments, anti-CCR8 antibody causes depletion of regulatory T (Treg) cells. In some embodiments, the anti-CCR8 antibody is selected from BMS-986340, FPA157, HFB1011, HBM1022, IO-1, IPG276, JTX-1811 (GS-1811), LM-108, S-531011, and SRF-114. In some embodiments, the anti-CCR8 antibody is JTX-1811 (GS-1811). In some embodiments, the subject is a human subject. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 97% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 98% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 99% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence selected from the group consisting of SEQ ID NOs: 1-18 and 21-27. In some embodiments, the fusion protein comprises an amino acid sequence that is at least about 97% identical to the amino acid sequence of SEQ ID NO: 14. In some embodiments, the fusion protein comprises an amino acid sequence that is at least about 98% identical to the amino acid sequence of SEQ ID NO: 14. In some embodiments, the fusion protein comprises an amino acid sequence that is at least about 99% identical to the amino acid sequence of SEQ ID NO: 14. In some embodiments, the fusion protein comprises the amino acid sequence of SEQ ID NO: 14.

Provided are methods of enhancing, promoting, and/or increasing the tumor infiltration of T-cells and/or NK cells in a subject in need thereof, comprising administering to the subject an effective amount of fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. Alternatively, in some embodiments, the method of enhancing, promoting, and/or increasing the tumor infiltration of T-cells and/or NK cells in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan. Alternatively, in some embodiments, the method of enhancing, promoting, and/or increasing the tumor infiltration of T-cells and/or NK cells in a subject in need thereof, comprising administering to the subject (I) an effective amount of a human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. Alternatively, in some embodiments, the method of enhancing, promoting, and/or increasing the tumor infiltration of T-cells and/or NK cells in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor. In some embodiments, the FLT3L modulator is a fusion protein comprising a FLT3L protein or fragment thereof and an Fc protein or fragment thereof. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18, 21-27, 114, and 115. In some embodiments, the Fc protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 111. In some embodiments, residues 13-17 of SEQ ID NO: 111 comprise the amino acid sequence PVAGT (SEQ ID NO: 116) and residue 76 of SEQ ID NO: 111 is a glycine. In some embodiments, the FLT3L protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NOs: 112, 113, or 117. In some embodiments, the FLT3L protein or fragment thereof comprises CDX-301, which is disclosed in International Publication No. 94/28391. In some embodiments, the FLT3L modulator comprises a FLT3L protein, Fc protein, or FLT3L-Fc fusion protein, or a fragment thereof as disclosed in International Publication No. 2022/031876. In some embodiments, the immunoconjugate is co-administered with the FLT3L modulator. In some embodiments, the FLT3L modulator comprises the amino acid sequence of any one of SEQ ID NOs: 101-105 and 107. In some embodiments, the immunoconjugate comprises datopotamab deruxtecan (DS-1062). In some embodiments, the FLT3L modulator comprises the amino acid sequence of any one of SEQ ID NOs: 111-115 and 117 or comprises CDX-301 and the immunoconjugate comprises datopotamab deruxtecan (DS-1062). In some embodiments, the effective amount of the fusion protein is at least about 200 μg, 225 μg, 300 μg, 500 μg, 600 μg, 675 μg, 700 μg, 800 μg, 900 μg, 1000 μg, 1200 μg, 1400 μg, 1600 μg, 1800 μg, or 2000 μg of the fusion protein per dose. In some embodiments, the effective amount of the fusion protein is between about 200 μg to about 30000 μg, about 200 μg to about 25000 μg, about 200 μg to about 20000 μg, about 500 μg to about 20000 μg, about 500 μg to about 15000 μg, about 500 μg to about 10000 μg, about 600 μg to about 20000 μg, about 600 μg to about 15000 μg, about 600 μg to about 10000 μg, about 600 μg to about 8000 μg, about 600 μg to about 5000 μg, about 600 μg to about 3000 μg, about 600 μg to about 2500 μg, about 600 μg to about 2000 μg of the fusion protein per dose. In some embodiments, the effective amount of the fusion protein is between about 200 μg to about to about 2500 μg of the fusion protein per dose. In some embodiments, the effective amount of the fusion protein is between about 600 μg to about 2000 μg of the fusion protein per dose. In some embodiments, the effective amount of the fusion protein is less than about 30000 μg, 25000 μg, 20000 μg, 15000 μg, 10000 μg, 9000 μg, 8000 μg, 7000 μg, 6000 μg, 5000 μg, 4000 μg, 3000 μg, or 2000 μg of the fusion protein per dose. In some embodiments, the effective amount of the fusion protein is less than about 25000 μg of the fusion protein per dose. In some embodiments, the effective amount of the fusion protein is less than about 20000 μg of the fusion protein per dose. In some embodiments, the effective amount of the fusion protein is less than about 15000 μg of the fusion protein per dose. In some embodiments, the effective amount of the fusion protein is less than about 10000 μg of the fusion protein per dose. In some embodiments, the effective amount of the fusion protein is less than about 5000 μg of the fusion protein per dose. In some embodiments, at least two doses of the fusion protein are administered at least two weeks apart. In some embodiments, at least two doses are administered 2 to 4 weeks apart. In some embodiments, at least two doses are administered at least about 2 weeks apart over a duration of at least about 1 month. In some embodiments, at least two doses are administered between about 2 to 5 weeks apart over a duration of between about 1 to 4 months. In some embodiments, between about 3 to about 8 doses of the fusion protein. In some embodiments, between about 3 to about 8 doses of the fusion protein, wherein at least two of the doses are administered between 2 to about 5 weeks apart over a duration of between 1 to 4 months. In some embodiments, the method further comprises pausing administration of the fusion protein for a period of at least about 6, 7, 8, 9, 10, 11, 12, 13, 14, 15, 16, 17, 18, 19, or 20 weeks. In some embodiments, the method further comprises pausing administration of the fusion protein for a period of up to about 3, 4, 5, 6, 7, 8, 9, 10, 11, 12, 13, 14, 15, 16, 17, 18, 19, or 20 months. In some embodiments, the method further comprises pausing administration of the fusion protein for a period of between about 6 weeks to about 8 months, about 2 to about 8 months, about 3 to about 8 months, about 2 to about 10 months, about 2 to about 6 months, about 3 to about 6 months, about 4 to about 8 months, or about 4 to about 6 months. In some embodiments, administration of the fusion protein is paused for at least about 6 weeks. In some embodiments, administration of the fusion protein is paused for at least about 10 weeks. In some embodiments, administration of the fusion protein is paused for at least about 12 weeks. In some embodiments, administration of the fusion protein is paused for at least about 16 weeks. In some embodiments, administration of the fusion protein is paused for up to about 10 months. In some embodiments, administration of the fusion protein is paused for up to about 9 months. In some embodiments, administration of the fusion protein is paused for up to about 8 months. In some embodiments, administration of the fusion protein is paused for up to about 7 months. In some embodiments, administration of the fusion protein is paused for up to about 6 months. In some embodiments, the method further comprises co-administering to the subject an anti-cancer agent. In some embodiments, the method further comprises co-administering to the subject an immunotherapy. In some embodiments, the method further comprises co-administering to the subject an immune checkpoint protein or receptor. In some embodiments, the method further comprises co-administering to the subject an inhibitor of an immune checkpoint protein or receptor. In some embodiments, the method further comprises co-administering to the subject an activator of an immune checkpoint protein or receptor. In some embodiments, the method further comprises co-administering to the subject sacituzumab govitecan. In some embodiments, the method further comprises co-administering to the subject an anti-CD47 antibody. In some embodiments, the method further comprises co-administering to the subject magrolimab. In some embodiments, the method further comprises co-administering to the subject an inhibitor of MCL-1. In some embodiments, the immunoconjugate is co-administered with the fusion protein. In some embodiments, the immunconjugate comprises an anti-Trop2-ADC. In some embodiments, the immunoconjugate comprises an anti-Trop2 antibody. In some embodiments, the FLT3R agonist is co-administered with the fusion protein. In some embodiments, the FLT3R agonist is selected from an antibody, small molecule, or cytokine. In some embodiments, the anti-PD1 antibody is co-administered with the fusion protein. In some embodiments, the anti-PD1 antibody is selected from balstilimab, budigalimab, camrelizumab, cemiplimab, cetrelimab, dostarlimab, genolimzumab, nivolumab, pembrolizumab, pidilizumab, prolgolimab, retifanlimab, sasanlimab, sintilimab, spartalizumab, tislelizumab, toripalimab, and zimberelimab. In some embodiments, the anti-PD1 antibody is zimberelimab. In some embodiments, an anti-PDL1 antibody is co-administered with the fusion protein. In some embodiments, the anti-PDL1 antibody is selected from atezolizumab, avelumab, cosibelimab, durvalumab, envafolimab, and lodapolimab. In some embodiments, the anti-PDL1 antibody is atezolizumab. In some embodiments, the anti-Tigit antibody is co-administered with the fusion protein. In some embodiments, the anti-Tigit antibody is selected from AB-308, AGEN-1307 (AGEN-1327), AGEN-1777, AK127, BMS-986207, domvanalimab, EOS-448, etigilimab, JS006, ociperlimab, SEA-TGT (SGN-TGT), tiragolumab, and vibostolimab. In some embodiments, the anti-Tigit antibody is vibostolimab. In some embodiments, the anti-Tigit antibody is AB-308. In some embodiments, the anti-Tigit antibody is domvanalimab. In some embodiments, MCL-1 inhibitor is co-administered with the fusion protein. In some embodiments, the MCL-1 inhibitor is selected from GS-9716, 564315 (MIK665), AZD5991, AMG-176, AMG-397, ABBV-467, and PRT1419. In some embodiments, the anti-CD47 antibody is co-administered with the fusion protein. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments, the adenosine pathway inhibitor is co-administered with the fusion protein. In some embodiments, the adenosine pathway inhibitor is selected from an adenosine receptor antagonist, CD39 inhibitor, and CD73 inhibitor. In some embodiments, the adenosine receptor antagonist is a small molecule. In some embodiments, the adenosine receptor antagonist is selected from etrumadenant (AB729; GS-0928), M1069, taminadenant, TT-4, and TT-10. In some embodiments, the adenosine receptor antagonist is AB729 (etrumadenant). In some embodiments, the CD39 inhibitor is selected from TTX-030, IPH5201, SRF617, nucleotide derivatives, anthraquinone derivatives, and suramin derivatives. In some embodiments, the anthraquinone derivate is RB2. In some embodiments, the CD73 inhibitor is a small molecule. In some embodiments, CD73 inhibitor is selected from AB680 (quemliclustat), AK131, ATG-037, BMS-986179, mupadolimab, NZV930, oleclumab, ORIC-533, PT-199, and uliledlimab. In some embodiments, the CD73 inhibitor is AB680 (quemliclustat). In some embodiments, the anti-CCR8 antibody is co-administered with the fusion protein. In some embodiments, anti-CCR8 antibody causes depletion of regulatory T (Treg) cells. In some embodiments, the anti-CCR8 antibody is selected from BMS-986340, FPA157, HFB1011, HBM1022, IO-1, IPG276, JTX-1811 (GS-1811), LM-108, S-531011, and SRF-114. In some embodiments, the anti-CCR8 antibody is JTX-1811 (GS-1811). In some embodiments, the subject is a human subject. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 97% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 98% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 99% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence selected from the group consisting of SEQ ID NOs: 1-18 and 21-27. In some embodiments, the fusion protein comprises an amino acid sequence that is at least about 97% identical to the amino acid sequence of SEQ ID NO: 14. In some embodiments, the fusion protein comprises an amino acid sequence that is at least about 98% identical to the amino acid sequence of SEQ ID NO: 14. In some embodiments, the fusion protein comprises an amino acid sequence that is at least about 99% identical to the amino acid sequence of SEQ ID NO: 14. In some embodiments, the fusion protein comprises the amino acid sequence of SEQ ID NO: 14.

Provided are methods of enhancing, promoting, and/or accelerating the recovery from or reversing the effects of lymphopenia in a subject in need thereof, comprising administering to the subject an effective amount of fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. Alternatively, in some embodiments, the method of enhancing, promoting, and/or accelerating the recovery from or reversing the effects of lymphopenia in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan. Alternatively, in some embodiments, the method of enhancing, promoting, and/or accelerating the recovery from or reversing the effects of lymphopenia in a subject in need thereof, comprising administering to the subject (I) an effective amount of a human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. Alternatively, in some embodiments, the method of enhancing, promoting, and/or accelerating the recovery from or reversing the effects of lymphopenia in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor. In some embodiments, the FLT3L modulator is a fusion protein comprising a FLT3L protein or fragment thereof and an Fc protein or fragment thereof. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18, 21-27, 114, and 115. In some embodiments, the Fc protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 111. In some embodiments, residues 13-17 of SEQ ID NO: 111 comprise the amino acid sequence PVAGT (SEQ ID NO: 116) and residue 76 of SEQ ID NO: 111 is a glycine. In some embodiments, the FLT3L protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NOs: 112, 113, or 117. In some embodiments, the FLT3L protein or fragment thereof comprises CDX-301, which is disclosed in International Publication No. 94/28391. In some embodiments, the FLT3L modulator comprises a FLT3L protein, Fc protein, or FLT3L-Fc fusion protein, or a fragment thereof as disclosed in International Publication No. 2022/031876. In some embodiments, the immunoconjugate is co-administered with the FLT3L modulator. In some embodiments, the FLT3L modulator comprises the amino acid sequence of any one of SEQ ID NOs: 101-105 and 107. In some embodiments, the immunoconjugate comprises datopotamab deruxtecan (DS-1062). In some embodiments, the FLT3L modulator comprises the amino acid sequence of any one of SEQ ID NOs: 111-115 and 117 or comprises CDX-301 and the immunoconjugate comprises datopotamab deruxtecan (DS-1062). In some embodiments, the effective amount of the fusion protein is at least about 200 μg, 225 μg, 300 μg, 500 μg, 600 μg, 675 μg, 700 μg, 800 μg, 900 μg, 1000 μg, 1200 μg, 1400 μg, 1600 μg, 1800 μg, or 2000 μg of the fusion protein per dose. In some embodiments, the effective amount of the fusion protein is between about 200 μg to about 30000 μg, about 200 μg to about 25000 μg, about 200 μg to about 20000 μg, about 500 μg to about 20000 μg, about 500 μg to about 15000 μg, about 500 μg to about 10000 μg, about 600 μg to about 20000 μg, about 600 μg to about 15000 μg, about 600 μg to about 10000 μg, about 600 μg to about 8000 μg, about 600 μg to about 5000 μg, about 600 μg to about 3000 μg, about 600 μg to about 2500 μg, about 600 μg to about 2000 μg of the fusion protein per dose. In some embodiments, the effective amount of the fusion protein is between about 200 μg to about to about 2500 μg of the fusion protein per dose. In some embodiments, the effective amount of the fusion protein is between about 600 μg to about 2000 μg of the fusion protein per dose. In some embodiments, the effective amount of the fusion protein is less than about 30000 μg, 25000 μg, 20000 μg, 15000 μg, 10000 μg, 9000 μg, 8000 μg, 7000 μg, 6000 μg, 5000 μg, 4000 μg, 3000 μg, or 2000 μg of the fusion protein per dose. In some embodiments, the effective amount of the fusion protein is less than about 25000 μg of the fusion protein per dose. In some embodiments, the effective amount of the fusion protein is less than about 20000 μg of the fusion protein per dose. In some embodiments, the effective amount of the fusion protein is less than about 15000 μg of the fusion protein per dose. In some embodiments, the effective amount of the fusion protein is less than about 10000 μg of the fusion protein per dose. In some embodiments, the effective amount of the fusion protein is less than about 5000 μg of the fusion protein per dose. In some embodiments, at least two doses of the fusion protein are administered at least two weeks apart. In some embodiments, at least two doses are administered 2 to 4 weeks apart. In some embodiments, at least two doses are administered at least about 2 weeks apart over a duration of at least about 1 month. In some embodiments, at least two doses are administered between about 2 to 5 weeks apart over a duration of between about 1 to 4 months. In some embodiments, between about 3 to about 8 doses of the fusion protein. In some embodiments, between about 3 to about 8 doses of the fusion protein, wherein at least two of the doses are administered between 2 to about 5 weeks apart over a duration of between 1 to 4 months. In some embodiments, the method further comprises pausing administration of the fusion protein fora period of at least about 6, 7, 8, 9, 10, 11, 12, 13, 14, 15, 16, 17, 18, 19, or 20 weeks. In some embodiments, the method further comprises pausing administration of the fusion protein fora period of up to about 3, 4, 5, 6, 7, 8, 9, 10, 11, 12, 13, 14, 15, 16, 17, 18, 19, or 20 months. In some embodiments, the method further comprises pausing administration of the fusion protein for a period of between about 6 weeks to about 8 months, about 2 to about 8 months, about 3 to about 8 months, about 2 to about 10 months, about 2 to about 6 months, about 3 to about 6 months, about 4 to about 8 months, or about 4 to about 6 months. In some embodiments, administration of the fusion protein is paused for at least about 6 weeks. In some embodiments, administration of the fusion protein is paused for at least about 10 weeks. In some embodiments, administration of the fusion protein is paused for at least about 12 weeks. In some embodiments, administration of the fusion protein is paused for at least about 16 weeks. In some embodiments, administration of the fusion protein is paused for up to about 10 months. In some embodiments, administration of the fusion protein is paused for up to about 9 months. In some embodiments, administration of the fusion protein is paused for up to about 8 months. In some embodiments, administration of the fusion protein is paused for up to about 7 months. In some embodiments, administration of the fusion protein is paused for up to about 6 months. In some embodiments, the method further comprises co-administering to the subject an anti-cancer agent. In some embodiments, the method further comprises co-administering to the subject an immunotherapy. In some embodiments, the method further comprises co-administering to the subject an immune checkpoint protein or receptor. In some embodiments, the method further comprises co-administering to the subject an inhibitor of an immune checkpoint protein or receptor. In some embodiments, the method further comprises co-administering to the subject an activator of an immune checkpoint protein or receptor. In some embodiments, the method further comprises co-administering to the subject sacituzumab govitecan. In some embodiments, the method further comprises co-administering to the subject an anti-CD47 antibody. In some embodiments, the method further comprises co-administering to the subject magrolimab. In some embodiments, the method further comprises co-administering to the subject an inhibitor of MCL-1. In some embodiments, the immunoconjugate is co-administered with the fusion protein. In some embodiments, the immunconjugate comprises an anti-Trop2-ADC. In some embodiments, the immunoconjugate comprises an anti-Trop2 antibody. In some embodiments, the FLT3R agonist is co-administered with the fusion protein. In some embodiments, the FLT3R agonist is selected from an antibody, small molecule, or cytokine. In some embodiments, the anti-PD1 antibody is co-administered with the fusion protein. In some embodiments, the anti-PD1 antibody is selected from balstilimab, budigalimab, camrelizumab, cemiplimab, cetrelimab, dostarlimab, genolimzumab, nivolumab, pembrolizumab, pidilizumab, prolgolimab, retifanlimab, sasanlimab, sintilimab, spartalizumab, tislelizumab, toripalimab, and zimberelimab. In some embodiments, the anti-PD1 antibody is zimberelimab. In some embodiments, an anti-PDL1 antibody is co-administered with the fusion protein. In some embodiments, the anti-PDL1 antibody is selected from atezolizumab, avelumab, cosibelimab, durvalumab, envafolimab, and lodapolimab. In some embodiments, the anti-PDL1 antibody is atezolizumab. In some embodiments, the anti-Tigit antibody is co-administered with the fusion protein. In some embodiments, the anti-Tigit antibody is selected from AB-308, AGEN-1307 (AGEN-1327), AGEN-1777, AK127, BMS-986207, domvanalimab, EOS-448, etigilimab, JS006, ociperlimab, SEA-TGT (SGN-TGT), tiragolumab, and vibostolimab. In some embodiments, the anti-Tigit antibody is vibostolimab. In some embodiments, the anti-Tigit antibody is AB-308. In some embodiments, the anti-Tigit antibody is domvanalimab. In some embodiments, MCL-1 inhibitor is co-administered with the fusion protein. In some embodiments, the MCL-1 inhibitor is selected from GS-9716, 564315 (MIK665), AZD5991, AMG-176, AMG-397, ABBV-467, and PRT1419. In some embodiments, the anti-CD47 antibody is co-administered with the fusion protein. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments, the adenosine pathway inhibitor is co-administered with the fusion protein. In some embodiments, the adenosine pathway inhibitor is selected from an adenosine receptor antagonist, CD39 inhibitor, and CD73 inhibitor. In some embodiments, the adenosine receptor antagonist is a small molecule. In some embodiments, the adenosine receptor antagonist is selected from etrumadenant (AB729; GS-0928), M1069, taminadenant, TT-4, and TT-10. In some embodiments, the adenosine receptor antagonist is AB729 (etrumadenant). In some embodiments, the CD39 inhibitor is selected from TTX-030, IPH5201, SRF617, nucleotide derivatives, anthraquinone derivatives, and suramin derivatives. In some embodiments, the anthraquinone derivate is RB2. In some embodiments, the CD73 inhibitor is a small molecule. In some embodiments, CD73 inhibitor is selected from AB680 (quemliclustat), AK131, ATG-037, BMS-986179, mupadolimab, NZV930, oleclumab, ORIC-533, PT-199, and uliledlimab. In some embodiments, the CD73 inhibitor is AB680 (quemliclustat). In some embodiments, the anti-CCR8 antibody is co-administered with the fusion protein. In some embodiments, anti-CCR8 antibody causes depletion of regulatory T (Treg) cells. In some embodiments, the anti-CCR8 antibody is selected from BMS-986340, FPA157, HFB1011, HBM1022, IO-1, IPG276, JTX-1811 (GS-1811), LM-108, S-531011, and SRF-114. In some embodiments, the anti-CCR8 antibody is JTX-1811 (GS-1811). In some embodiments, the subject is a human subject. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 97% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 98% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 99% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence selected from the group consisting of SEQ ID NOs: 1-18 and 21-27. In some embodiments, the fusion protein comprises an amino acid sequence that is at least about 97% identical to the amino acid sequence of SEQ ID NO: 14. In some embodiments, the fusion protein comprises an amino acid sequence that is at least about 98% identical to the amino acid sequence of SEQ ID NO: 14. In some embodiments, the fusion protein comprises an amino acid sequence that is at least about 99% identical to the amino acid sequence of SEQ ID NO: 14. In some embodiments, the fusion protein comprises the amino acid sequence of SEQ ID NO: 14.

Provided are methods of enhancing, improving, and/or increasing the response to an anticancer therapy in a subject in need thereof, comprising administering to the subject (I) an effective amount of fusion protein comprising a human fms related tyrosine kinase 3 ligand (FLT3L) extracellular domain operably linked to an immunoglobulin fragment crystallizable region (Fc region); and (II) an effective amount of an anticancer agent, wherein (a) at least 5 amino acids are truncated from the C-terminus of the FLT3L extracellular domain; and/or (b) the Fc region does not comprise a hinge region. In some embodiments, the effective amount of the fusion protein is at least about 200 μg, 225 μg, 300 μg, 500 μg, 600 μg, 675 μg, 700 μg, 800 μg, 900 μg, 1000 μg, 1200 μg, 1400 μg, 1600 μg, 1800 μg, or 2000 μg of the fusion protein per dose. In some embodiments, the effective amount of the fusion protein is between about 200 μg to about 30000 μg, about 200 μg to about 25000 μg, about 200 μg to about 20000 μg, about 500 μg to about 20000 μg, about 500 μg to about 15000 μg, about 500 μg to about 10000 μg, about 600 μg to about 20000 μg, about 600 μg to about 15000 μg, about 600 μg to about 10000 μg, about 600 μg to about 8000 μg, about 600 μg to about 5000 μg, about 600 μg to about 3000 μg, about 600 μg to about 2500 μg, about 600 μg to about 2000 μg of the fusion protein per dose. In some embodiments, the effective amount of the fusion protein is between about 200 μg to about to about 2500 μg of the fusion protein per dose. In some embodiments, the effective amount of the fusion protein is between about 600 μg to about 2000 μg of the fusion protein per dose. In some embodiments, the effective amount of the fusion protein is less than about 30000 μg, 25000 μg, 20000 μg, 15000 μg, 10000 μg, 9000 μg, 8000 μg, 7000 μg, 6000 μg, 5000 μg, 4000 μg, 3000 μg, or 2000 μg of the fusion protein per dose. In some embodiments, the effective amount of the fusion protein is less than about 25000 μg of the fusion protein per dose. In some embodiments, the effective amount of the fusion protein is less than about 20000 μg of the fusion protein per dose. In some embodiments, the effective amount of the fusion protein is less than about 15000 μg of the fusion protein per dose. In some embodiments, the effective amount of the fusion protein is less than about 10000 μg of the fusion protein per dose. In some embodiments, the effective amount of the fusion protein is less than about 5000 μg of the fusion protein per dose. In some embodiments, at least two doses of the fusion protein are administered at least two weeks apart. In some embodiments, at least two doses are administered 2 to 4 weeks apart. In some embodiments, at least two doses are administered at least about 2 weeks apart over a duration of at least about 1 month. In some embodiments, at least two doses are administered between about 2 to 5 weeks apart over a duration of between about 1 to 4 months. In some embodiments, between about 3 to about 8 doses of the fusion protein. In some embodiments, between about 3 to about 8 doses of the fusion protein, wherein at least two of the doses are administered between 2 to about 5 weeks apart over a duration of between 1 to 4 months. In some embodiments, the method further comprises pausing administration of the fusion protein for a period of at least about 6, 7, 8, 9, 10, 11, 12, 13, 14, 15, 16, 17, 18, 19, or 20 weeks. In some embodiments, the method further comprises pausing administration of the fusion protein for a period of up to about 3, 4, 5, 6, 7, 8, 9, 10, 11, 12, 13, 14, 15, 16, 17, 18, 19, or 20 months. In some embodiments, the method further comprises pausing administration of the fusion protein for a period of between about 6 weeks to about 8 months, about 2 to about 8 months, about 3 to about 8 months, about 2 to about 10 months, about 2 to about 6 months, about 3 to about 6 months, about 4 to about 8 months, or about 4 to about 6 months. In some embodiments, administration of the fusion protein is paused for at least about 6 weeks. In some embodiments, administration of the fusion protein is paused for at least about 10 weeks. In some embodiments, administration of the fusion protein is paused for at least about 12 weeks. In some embodiments, administration of the fusion protein is paused for at least about 16 weeks. In some embodiments, administration of the fusion protein is paused for up to about 10 months. In some embodiments, administration of the fusion protein is paused for up to about 9 months. In some embodiments, administration of the fusion protein is paused for up to about 8 months. In some embodiments, administration of the fusion protein is paused for up to about 7 months. In some embodiments, administration of the fusion protein is paused for up to about 6 months. In some embodiments, the method further comprises co-administering to the subject an immunotherapy. In some embodiments, the method further comprises co-administering to the subject an immune checkpoint protein or receptor. In some embodiments, the method further comprises co-administering to the subject an inhibitor of an immune checkpoint protein or receptor. In some embodiments, the method further comprises co-administering to the subject an activator of an immune checkpoint protein or receptor. In some embodiments, the anti-cancer agent is sacituzumab govitecan. In some embodiments, the anti-cancer agent is an anti-CD47 antibody. In some embodiments, the anti-cancer agent is magrolimab. In some embodiments, the anti-cancer agent is an inhibitor of MCL-1. In some embodiments, the subject is a human subject. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 97% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 98% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 99% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence selected from the group consisting of SEQ ID NOs: 1-18 and 21-27. In some embodiments, the fusion protein comprises an amino acid sequence that is at least about 97% identical to the amino acid sequence of SEQ ID NO: 14. In some embodiments, the fusion protein comprises an amino acid sequence that is at least about 98% identical to the amino acid sequence of SEQ ID NO: 14. In some embodiments, the fusion protein comprises an amino acid sequence that is at least about 99% identical to the amino acid sequence of SEQ ID NO: 14. In some embodiments, the fusion protein comprises the amino acid sequence of SEQ ID NO: 14.

Provided are methods of promoting, inducing and/or increasing the expansion and/or proliferation of a cell or a population of cells that express fms related tyrosine kinase 3 (FLT3, CD135) in a subject in need thereof, comprising administering to the subject an effective amount of fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. Alternatively, in some embodiments, the method of promoting, inducing and/or increasing the expansion and/or proliferation of a cell or a population of cells that express fms related tyrosine kinase 3 (FLT3, CD135) in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan. Alternatively, in some embodiments, the method of promoting, inducing and/or increasing the expansion and/or proliferation of a cell or a population of cells that express fms related tyrosine kinase 3 (FLT3, CD135) in a subject in need thereof, comprising administering to the subject (I) an effective amount of a human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. Alternatively, in some embodiments, the method of promoting, inducing and/or increasing the expansion and/or proliferation of a cell or a population of cells that express fms related tyrosine kinase 3 (FLT3, CD135) in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor. In some embodiments, the FLT3L modulator is a fusion protein comprising a FLT3L protein or fragment thereof and an Fc protein or fragment thereof. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18, 21-27, 114, and 115. In some embodiments, the Fc protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 111. In some embodiments, residues 13-17 of SEQ ID NO: 111 comprise the amino acid sequence PVAGT (SEQ ID NO: 116) and residue 76 of SEQ ID NO: 111 is a glycine. In some embodiments, the FLT3L protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NOs: 112, 113, or 117. In some embodiments, the FLT3L protein or fragment thereof comprises CDX-301, which is disclosed in International Publication No. 94/28391. In some embodiments, the FLT3L modulator comprises a FLT3L protein, Fc protein, or FLT3L-Fc fusion protein, or a fragment thereof as disclosed in International Publication No. 2022/031876. In some embodiments, the immunoconjugate is co-administered with the FLT3L modulator. In some embodiments, the FLT3L modulator comprises the amino acid sequence of any one of SEQ ID NOs: 101-105 and 107. In some embodiments, the immunoconjugate comprises datopotamab deruxtecan (DS-1062). In some embodiments, the FLT3L modulator comprises the amino acid sequence of any one of SEQ ID NOs: 111-115 and 117 or comprises CDX-301 and the immunoconjugate comprises datopotamab deruxtecan (DS-1062). In some embodiments, the effective amount of the fusion protein is at least about 200 μg, 225 μg, 300 μg, 500 μg, 600 μg, 675 μg, 700 μg, 800 μg, 900 μg, 1000 μg, 1200 μg, 1400 μg, 1600 μg, 1800 μg, or 2000 μg of the fusion protein per dose. In some embodiments, the effective amount of the fusion protein is between about 200 μg to about 30000 μg, about 200 μg to about 25000 μg, about 200 μg to about 20000 μg, about 500 μg to about 20000 μg, about 500 μg to about 15000 μg, about 500 μg to about 10000 μg, about 600 μg to about 20000 μg, about 600 μg to about 15000 μg, about 600 μg to about 10000 μg, about 600 μg to about 8000 μg, about 600 μg to about 5000 μg, about 600 μg to about 3000 μg, about 600 μg to about 2500 μg, about 600 μg to about 2000 μg of the fusion protein per dose. In some embodiments, the effective amount of the fusion protein is between about 200 μg to about to about 2500 μg of the fusion protein per dose. In some embodiments, the effective amount of the fusion protein is between about 600 μg to about 2000 μg of the fusion protein per dose. In some embodiments, the effective amount of the fusion protein is less than about 30000 μg, 25000 μg, 20000 μg, 15000 μg, 10000 μg, 9000 μg, 8000 μg, 7000 μg, 6000 μg, 5000 μg, 4000 μg, 3000 μg, or 2000 μg of the fusion protein per dose. In some embodiments, the effective amount of the fusion protein is less than about 25000 μg of the fusion protein per dose. In some embodiments, the effective amount of the fusion protein is less than about 20000 μg of the fusion protein per dose. In some embodiments, the effective amount of the fusion protein is less than about 15000 μg of the fusion protein per dose. In some embodiments, the effective amount of the fusion protein is less than about 10000 μg of the fusion protein per dose. In some embodiments, the effective amount of the fusion protein is less than about 5000 μg of the fusion protein per dose. In some embodiments, at least two doses of the fusion protein are administered at least two weeks apart. In some embodiments, at least two doses are administered 2 to 4 weeks apart. In some embodiments, at least two doses are administered at least about 2 weeks apart over a duration of at least about 1 month. In some embodiments, at least two doses are administered between about 2 to 5 weeks apart over a duration of between about 1 to 4 months. In some embodiments, between about 3 to about 8 doses of the fusion protein. In some embodiments, between about 3 to about 8 doses of the fusion protein, wherein at least two of the doses are administered between 2 to about 5 weeks apart over a duration of between 1 to 4 months. In some embodiments, the method further comprises pausing administration of the fusion protein fora period of at least about 6, 7, 8, 9, 10, 11, 12, 13, 14, 15, 16, 17, 18, 19, or 20 weeks. In some embodiments, the method further comprises pausing administration of the fusion protein for a period of up to about 3, 4, 5, 6, 7, 8, 9, 10, 11, 12, 13, 14, 15, 16, 17, 18, 19, or 20 months. In some embodiments, the method further comprises pausing administration of the fusion protein for a period of between about 6 weeks to about 8 months, about 2 to about 8 months, about 3 to about 8 months, about 2 to about 10 months, about 2 to about 6 months, about 3 to about 6 months, about 4 to about 8 months, or about 4 to about 6 months. In some embodiments, administration of the fusion protein is paused for at least about 6 weeks. In some embodiments, administration of the fusion protein is paused for at least about 10 weeks. In some embodiments, administration of the fusion protein is paused for at least about 12 weeks. In some embodiments, administration of the fusion protein is paused for at least about 16 weeks. In some embodiments, administration of the fusion protein is paused for up to about 10 months. In some embodiments, administration of the fusion protein is paused for up to about 9 months. In some embodiments, administration of the fusion protein is paused for up to about 8 months. In some embodiments, administration of the fusion protein is paused for up to about 7 months. In some embodiments, administration of the fusion protein is paused for up to about 6 months. In some embodiments, the method further comprises co-administering to the subject an anti-cancer agent. In some embodiments, the method further comprises co-administering to the subject an immunotherapy. In some embodiments, the method further comprises co-administering to the subject an immune checkpoint protein or receptor. In some embodiments, the method further comprises co-administering to the subject an inhibitor of an immune checkpoint protein or receptor. In some embodiments, the method further comprises co-administering to the subject an activator of an immune checkpoint protein or receptor. In some embodiments, the method further comprises co-administering to the subject sacituzumab govitecan. In some embodiments, the method further comprises co-administering to the subject an anti-CD47 antibody. In some embodiments, the method further comprises co-administering to the subject magrolimab. In some embodiments, the method further comprises co-administering to the subject an inhibitor of MCL-1. In some embodiments, the immunoconjugate is co-administered with the fusion protein. In some embodiments, the immunconjugate comprises an anti-Trop2-ADC. In some embodiments, the immunoconjugate comprises an anti-Trop2 antibody. In some embodiments, the FLT3R agonist is co-administered with the fusion protein. In some embodiments, the FLT3R agonist is selected from an antibody, small molecule, or cytokine. In some embodiments, the anti-PD1 antibody is co-administered with the fusion protein. In some embodiments, the anti-PD1 antibody is selected from balstilimab, budigalimab, camrelizumab, cemiplimab, cetrelimab, dostarlimab, genolimzumab, nivolumab, pembrolizumab, pidilizumab, prolgolimab, retifanlimab, sasanlimab, sintilimab, spartalizumab, tislelizumab, toripalimab, and zimberelimab. In some embodiments, the anti-PD1 antibody is zimberelimab. In some embodiments, an anti-PDL1 antibody is co-administered with the fusion protein. In some embodiments, the anti-PDL1 antibody is selected from atezolizumab, avelumab, cosibelimab, durvalumab, envafolimab, and lodapolimab. In some embodiments, the anti-PDL1 antibody is atezolizumab. In some embodiments, the anti-Tigit antibody is co-administered with the fusion protein. In some embodiments, the anti-Tigit antibody is selected from AB-308, AGEN-1307 (AGEN-1327), AGEN-1777, AK127, BMS-986207, domvanalimab, EOS-448, etigilimab, JS006, ociperlimab, SEA-TGT (SGN-TGT), tiragolumab, and vibostolimab. In some embodiments, the anti-Tigit antibody is vibostolimab. In some embodiments, the anti-Tigit antibody is AB-308. In some embodiments, the anti-Tigit antibody is domvanalimab. In some embodiments, MCL-1 inhibitor is co-administered with the fusion protein. In some embodiments, the MCL-1 inhibitor is selected from GS-9716, 564315 (MIK665), AZD5991, AMG-176, AMG-397, ABBV-467, and PRT1419. In some embodiments, the anti-CD47 antibody is co-administered with the fusion protein. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments, the adenosine pathway inhibitor is co-administered with the fusion protein. In some embodiments, the adenosine pathway inhibitor is selected from an adenosine receptor antagonist, CD39 inhibitor, and CD73 inhibitor. In some embodiments, the adenosine receptor antagonist is a small molecule. In some embodiments, the adenosine receptor antagonist is selected from etrumadenant (AB729; GS-0928), M1069, taminadenant, TT-4, and TT-10. In some embodiments, the adenosine receptor antagonist is AB729 (etrumadenant). In some embodiments, the CD39 inhibitor is selected from TTX-030, IPH5201, SRF617, nucleotide derivatives, anthraquinone derivatives, and suramin derivatives. In some embodiments, the anthraquinone derivate is RB2. In some embodiments, the CD73 inhibitor is a small molecule. In some embodiments, CD73 inhibitor is selected from AB680 (quemliclustat), AK131, ATG-037, BMS-986179, mupadolimab, NZV930, oleclumab, ORIC-533, PT-199, and uliledlimab. In some embodiments, the CD73 inhibitor is AB680 (quemliclustat). In some embodiments, the anti-CCR8 antibody is co-administered with the fusion protein. In some embodiments, anti-CCR8 antibody causes depletion of regulatory T (Treg) cells. In some embodiments, the anti-CCR8 antibody is selected from BMS-986340, FPA157, HFB1011, HBM1022, IO-1, IPG276, JTX-1811 (GS-1811), LM-108, S-531011, and SRF-114. In some embodiments, the anti-CCR8 antibody is JTX-1811 (GS-1811). In some embodiments, the subject is a human subject. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 97% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 98% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 99% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence selected from the group consisting of SEQ ID NOs: 1-18 and 21-27. In some embodiments, the fusion protein comprises an amino acid sequence that is at least about 97% identical to the amino acid sequence of SEQ ID NO: 14. In some embodiments, the fusion protein comprises an amino acid sequence that is at least about 98% identical to the amino acid sequence of SEQ ID NO: 14. In some embodiments, the fusion protein comprises an amino acid sequence that is at least about 99% identical to the amino acid sequence of SEQ ID NO: 14. In some embodiments, the fusion protein comprises the amino acid sequence of SEQ ID NO: 14.

Provided are methods of enhancing, improving, and/or increasing the response to an immunotherapyin a subject in need thereof, comprising administering to the subject (I) an effective amount of fusion protein comprising a human fms related tyrosine kinase 3 ligand (FLT3L) extracellular domain operably linked to an immunoglobulin fragment crystallizable region (Fc region); and (II) an effective amount of an immunotherapy, wherein (a) at least 5 amino acids are truncated from the C-terminus of the FLT3L extracellular domain; and/or (b) the Fc region does not comprise a hinge region. In some embodiments, the effective amount of the fusion protein is at least about 200 μg, 225 μg, 300 μg, 500 μg, 600 μg, 675 μg, 700 μg, 800 μg, 900 μg, 1000 μg, 1200 μg, 1400 μg, 1600 μg, 1800 μg, or 2000 μg of the fusion protein per dose. In some embodiments, the effective amount of the fusion protein is between about 200 μg to about 30000 μg, about 200 μg to about 25000 μg, about 200 μg to about 20000 μg, about 500 μg to about 20000 μg, about 500 μg to about 15000 μg, about 500 μg to about 10000 μg, about 600 μg to about 20000 μg, about 600 μg to about 15000 μg, about 600 μg to about 10000 μg, about 600 μg to about 8000 μg, about 600 μg to about 5000 μg, about 600 μg to about 3000 μg, about 600 μg to about 2500 μg, about 600 μg to about 2000 μg of the fusion protein per dose. In some embodiments, the effective amount of the fusion protein is between about 200 μg to about to about 2500 μg of the fusion protein per dose. In some embodiments, the effective amount of the fusion protein is between about 600 μg to about 2000 μg of the fusion protein per dose. In some embodiments, the effective amount of the fusion protein is less than about 30000 μg, 25000 μg, 20000 μg, 15000 μg, 10000 μg, 9000 μg, 8000 μg, 7000 μg, 6000 μg, 5000 μg, 4000 μg, 3000 μg, or 2000 μg of the fusion protein per dose. In some embodiments, the effective amount of the fusion protein is less than about 25000 μg of the fusion protein per dose. In some embodiments, the effective amount of the fusion protein is less than about 20000 μg of the fusion protein per dose. In some embodiments, the effective amount of the fusion protein is less than about 15000 μg of the fusion protein per dose. In some embodiments, the effective amount of the fusion protein is less than about 10000 μg of the fusion protein per dose. In some embodiments, the effective amount of the fusion protein is less than about 5000 μg of the fusion protein per dose. In some embodiments, at least two doses of the fusion protein are administered at least two weeks apart. In some embodiments, at least two doses are administered 2 to 4 weeks apart. In some embodiments, at least two doses are administered at least about 2 weeks apart over a duration of at least about 1 month. In some embodiments, at least two doses are administered between about 2 to 5 weeks apart over a duration of between about 1 to 4 months. In some embodiments, between about 3 to about 8 doses of the fusion protein. In some embodiments, between about 3 to about 8 doses of the fusion protein, wherein at least two of the doses are administered between 2 to about 5 weeks apart over a duration of between 1 to 4 months. In some embodiments, the method further comprises pausing administration of the fusion protein for a period of at least about 6, 7, 8, 9, 10, 11, 12, 13, 14, 15, 16, 17, 18, 19, or 20 weeks. In some embodiments, the method further comprises pausing administration of the fusion protein for a period of up to about 3, 4, 5, 6, 7, 8, 9, 10, 11, 12, 13, 14, 15, 16, 17, 18, 19, or 20 months. In some embodiments, the method further comprises pausing administration of the fusion protein for a period of between about 6 weeks to about 8 months, about 2 to about 8 months, about 3 to about 8 months, about 2 to about 10 months, about 2 to about 6 months, about 3 to about 6 months, about 4 to about 8 months, or about 4 to about 6 months. In some embodiments, administration of the fusion protein is paused for at least about 6 weeks. In some embodiments, administration of the fusion protein is paused for at least about 10 weeks. In some embodiments, administration of the fusion protein is paused for at least about 12 weeks. In some embodiments, administration of the fusion protein is paused for at least about 16 weeks. In some embodiments, administration of the fusion protein is paused for up to about 10 months. In some embodiments, administration of the fusion protein is paused for up to about 9 months. In some embodiments, administration of the fusion protein is paused for up to about 8 months. In some embodiments, administration of the fusion protein is paused for up to about 7 months. In some embodiments, administration of the fusion protein is paused for up to about 6 months. In some embodiments, the method further comprises co-administering to the subject an anti-cancer agent. In some embodiments, the method further comprises co-administering to the subject an immune checkpoint protein or receptor. In some embodiments, the method further comprises co-administering to the subject an inhibitor of an immune checkpoint protein or receptor. In some embodiments, the method further comprises co-administering to the subject an activator of an immune checkpoint protein or receptor. In some embodiments, the method further comprises co-administering to the subject sacituzumab govitecan. In some embodiments, the method further comprises co-administering to the subject an anti-CD47 antibody. In some embodiments, the method further comprises co-administering to the subject magrolimab. In some embodiments, the method further comprises co-administering to the subject an inhibitor of MCL-1. In some embodiments, the immunoconjugate is co-administered with the fusion protein. In some embodiments, the immunconjugate comprises an anti-Trop2-ADC. In some embodiments, the immunoconjugate comprises an anti-Trop2 antibody. In some embodiments, the FLT3R agonist is co-administered with the fusion protein. In some embodiments, the FLT3R agonist is selected from an antibody, small molecule, or cytokine. In some embodiments, the anti-PD1 antibody is co-administered with the fusion protein. In some embodiments, the anti-PD1 antibody is selected from balstilimab, budigalimab, camrelizumab, cemiplimab, cetrelimab, dostarlimab, genolimzumab, nivolumab, pembrolizumab, pidilizumab, prolgolimab, retifanlimab, sasanlimab, sintilimab, spartalizumab, tislelizumab, toripalimab, and zimberelimab. In some embodiments, the anti-PD1 antibody is zimberelimab. In some embodiments, an anti-PDL1 antibody is co-administered with the fusion protein. In some embodiments, the anti-PDL1 antibody is selected from atezolizumab, avelumab, cosibelimab, durvalumab, envafolimab, and lodapolimab. In some embodiments, the anti-PDL1 antibody is atezolizumab. In some embodiments, the anti-Tigit antibody is co-administered with the fusion protein. In some embodiments, the anti-Tigit antibody is selected from AB-308, AGEN-1307 (AGEN-1327), AGEN-1777, AK127, BMS-986207, domvanalimab, EOS-448, etigilimab, JS006, ociperlimab, SEA-TGT (SGN-TGT), tiragolumab, and vibostolimab. In some embodiments, the anti-Tigit antibody is vibostolimab. In some embodiments, the anti-Tigit antibody is AB-308. In some embodiments, the anti-Tigit antibody is domvanalimab. In some embodiments, MCL-1 inhibitor is co-administered with the fusion protein. In some embodiments, the MCL-1 inhibitor is selected from GS-9716, 564315 (MIK665), AZD5991, AMG-176, AMG-397, ABBV-467, and PRT1419. In some embodiments, the anti-CD47 antibody is co-administered with the fusion protein. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments, the adenosine pathway inhibitor is co-administered with the fusion protein. In some embodiments, the adenosine pathway inhibitor is selected from an adenosine receptor antagonist, CD39 inhibitor, and CD73 inhibitor. In some embodiments, the adenosine receptor antagonist is a small molecule. In some embodiments, the adenosine receptor antagonist is selected from etrumadenant (AB729; GS-0928), M1069, taminadenant, TT-4, and TT-10. In some embodiments, the adenosine receptor antagonist is AB729 (etrumadenant). In some embodiments, the CD39 inhibitor is selected from TTX-030, IPH5201, SRF617, nucleotide derivatives, anthraquinone derivatives, and suramin derivatives. In some embodiments, the anthraquinone derivate is RB2. In some embodiments, the CD73 inhibitor is a small molecule. In some embodiments, CD73 inhibitor is selected from AB680 (quemliclustat), AK131, ATG-037, BMS-986179, mupadolimab, NZV930, oleclumab, ORIC-533, PT-199, and uliledlimab. In some embodiments, the CD73 inhibitor is AB680 (quemliclustat). In some embodiments, the anti-CCR8 antibody is co-administered with the fusion protein. In some embodiments, anti-CCR8 antibody causes depletion of regulatory T (Treg) cells. In some embodiments, the anti-CCR8 antibody is selected from BMS-986340, FPA157, HFB1011, HBM1022, IO-1, IPG276, JTX-1811 (GS-1811), LM-108, S-531011, and SRF-114. In some embodiments, the anti-CCR8 antibody is JTX-1811 (GS-1811). In some embodiments, the subject is a human subject. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 97% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 98% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 99% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence selected from the group consisting of SEQ ID NOs: 1-18 and 21-27. In some embodiments, the fusion protein comprises an amino acid sequence that is at least about 97% identical to the amino acid sequence of SEQ ID NO: 14. In some embodiments, the fusion protein comprises an amino acid sequence that is at least about 98% identical to the amino acid sequence of SEQ ID NO: 14. In some embodiments, the fusion protein comprises an amino acid sequence that is at least about 99% identical to the amino acid sequence of SEQ ID NO: 14. In some embodiments, the fusion protein comprises the amino acid sequence of SEQ ID NO: 14.

Provided are methods of inducing the immune system in a subject in need thereof, comprising administering to the subject an effective amount of fusion protein comprising a human fms related tyrosine kinase 3 ligand (FLT3L) extracellular domain operably linked to an immunoglobulin fragment crystallizable region (Fc region), wherein (a) at least 5 amino acids are truncated from the C-terminus of the FLT3L extracellular domain; and/or (b) the Fc region does not comprise a hinge region. Alternatively, in some embodiments, the method of inducing an immune system in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan. Alternatively, in some embodiments, the method of inducing the immune system in a subject in need thereof, comprising administering to the subject (I) an effective amount of a human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. Alternatively, in some embodiments, the method of inducing the immune system in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor. In some embodiments, the adenosine pathway inhibitor is selected from an adenosine receptor antagonist, CD39 inhibitor, and CD73 inhibitor. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18, 21-27, 114, and 115. In some embodiments, the Fc protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 111. In some embodiments, residues 13-17 of SEQ ID NO: 111 comprise the amino acid sequence PVAGT (SEQ ID NO: 116) and residue 76 of SEQ ID NO: 111 is a glycine. In some embodiments, the FLT3L protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NOs: 112, 113, or 117. In some embodiments, the FLT3L protein or fragment thereof comprises CDX-301, which is disclosed in International Publication No. 94/28391. In some embodiments, the FLT3L modulator comprises a FLT3L protein, Fc protein, or FLT3L-Fc fusion protein, or a fragment thereof as disclosed in International Publication No. 2022/031876. In some embodiments, the immunoconjugate is co-administered with the FLT3L modulator. In some embodiments, the FLT3L modulator comprises the amino acid sequence of any one of SEQ ID NOs: 101-105 and 107. In some embodiments, the immunoconjugate comprises datopotamab deruxtecan (DS-1062). In some embodiments, the FLT3L modulator comprises the amino acid sequence of any one of SEQ ID NOs: 111-115 and 117 or comprises CDX-301 and the immunoconjugate comprises datopotamab deruxtecan (DS-1062). In some embodiments, the effective amount of the fusion protein is between about 200 μg to about 30000 μg, about 200 μg to about 25000 μg, about 200 μg to about 20000 μg, about 500 μg to about 20000 μg, about 500 μg to about 15000 μg, about 500 μg to about 10000 μg, about 600 μg to about 20000 μg, about 600 μg to about 15000 μg, about 600 μg to about 10000 μg, about 600 μg to about 8000 μg, about 600 μg to about 5000 μg, about 600 μg to about 3000 μg, about 600 μg to about 2500 μg, about 600 μg to about 2000 μg of the fusion protein per dose. In some embodiments, the effective amount of the fusion protein is between about 200 μg to about to about 2500 μg of the fusion protein per dose. In some embodiments, the effective amount of the fusion protein is between about 600 μg to about 2000 μg of the fusion protein per dose. In some embodiments, the effective amount of the fusion protein is less than about 30000 μg, 25000 μg, 20000 μg, 15000 μg, 10000 μg, 9000 μg, 8000 μg, 7000 μg, 6000 μg, 5000 μg, 4000 μg, 3000 μg, or 2000 μg of the fusion protein per dose. In some embodiments, the effective amount of the fusion protein is less than about 25000 μg of the fusion protein per dose. In some embodiments, the effective amount of the fusion protein is less than about 20000 μg of the fusion protein per dose. In some embodiments, the effective amount of the fusion protein is less than about 15000 μg of the fusion protein per dose. In some embodiments, the effective amount of the fusion protein is less than about 10000 μg of the fusion protein per dose. In some embodiments, the effective amount of the fusion protein is less than about 5000 μg of the fusion protein per dose. In some embodiments, at least two doses of the fusion protein are administered at least two weeks apart. In some embodiments, at least two doses are administered 2 to 4 weeks apart. In some embodiments, at least two doses are administered at least about 2 weeks apart over a duration of at least about 1 month. In some embodiments, at least two doses are administered between about 2 to 5 weeks apart over a duration of between about 1 to 4 months. In some embodiments, between about 3 to about 8 doses of the fusion protein. In some embodiments, between about 3 to about 8 doses of the fusion protein, wherein at least two of the doses are administered between 2 to about 5 weeks apart over a duration of between 1 to 4 months. In some embodiments, the method further comprises pausing administration of the fusion protein for a period of at least about 6, 7, 8, 9, 10, 11, 12, 13, 14, 15, 16, 17, 18, 19, or 20 weeks. In some embodiments, the method further comprises pausing administration of the fusion protein for a period of up to about 3, 4, 5, 6, 7, 8, 9, 10, 11, 12, 13, 14, 15, 16, 17, 18, 19, or 20 months. In some embodiments, the method further comprises pausing administration of the fusion protein for a period of between about 6 weeks to about 8 months, about 2 to about 8 months, about 3 to about 8 months, about 2 to about 10 months, about 2 to about 6 months, about 3 to about 6 months, about 4 to about 8 months, or about 4 to about 6 months. In some embodiments, administration of the fusion protein is paused for at least about 6 weeks. In some embodiments, administration of the fusion protein is paused for at least about 10 weeks. In some embodiments, administration of the fusion protein is paused for at least about 12 weeks. In some embodiments, administration of the fusion protein is paused for at least about 16 weeks. In some embodiments, administration of the fusion protein is paused for up to about 10 months. In some embodiments, administration of the fusion protein is paused for up to about 9 months. In some embodiments, administration of the fusion protein is paused for up to about 8 months. In some embodiments, administration of the fusion protein is paused for up to about 7 months. In some embodiments, administration of the fusion protein is paused for up to about 6 months. In some embodiments, the method further comprises co-administering to the subject an anti-cancer agent. In some embodiments, the method further comprises co-administering to the subject an immunotherapy. In some embodiments, the method further comprises co-administering to the subject an immune checkpoint protein or receptor. In some embodiments, the method further comprises co-administering to the subject an inhibitor of an immune checkpoint protein or receptor. In some embodiments, the method further comprises co-administering to the subject an activator of an immune checkpoint protein or receptor. In some embodiments, the method further comprises co-administering to the subject sacituzumab govitecan. In some embodiments, the method further comprises co-administering to the subject an anti-CD47 antibody. In some embodiments, the method further comprises co-administering to the subject magrolimab. In some embodiments, the method further comprises co-administering to the subject an inhibitor of MCL-1. In some embodiments, the immunoconjugate is co-administered with the fusion protein. In some embodiments, the immunconjugate comprises an anti-Trop2-ADC. In some embodiments, the immunoconjugate comprises an anti-Trop2 antibody. In some embodiments, the FLT3R agonist is co-administered with the fusion protein. In some embodiments, the FLT3R agonist is selected from an antibody, small molecule, or cytokine. In some embodiments, the anti-PD1 antibody is co-administered with the fusion protein. In some embodiments, the anti-PD1 antibody is selected from balstilimab, budigalimab, camrelizumab, cemiplimab, cetrelimab, dostarlimab, genolimzumab, nivolumab, pembrolizumab, pidilizumab, prolgolimab, retifanlimab, sasanlimab, sintilimab, spartalizumab, tislelizumab, toripalimab, and zimberelimab. In some embodiments, the anti-PD1 antibody is zimberelimab. In some embodiments, an anti-PDL1 antibody is co-administered with the fusion protein. In some embodiments, the anti-PDL1 antibody is selected from atezolizumab, avelumab, cosibelimab, durvalumab, envafolimab, and lodapolimab. In some embodiments, the anti-PDL1 antibody is atezolizumab. In some embodiments, the anti-Tigit antibody is co-administered with the fusion protein. In some embodiments, the anti-Tigit antibody is selected from AB-308, AGEN-1307 (AGEN-1327), AGEN-1777, AK127, BMS-986207, domvanalimab, EOS-448, etigilimab, JS006, ociperlimab, SEA-TGT (SGN-TGT), tiragolumab, and vibostolimab. In some embodiments, the anti-Tigit antibody is vibostolimab. In some embodiments, the anti-Tigit antibody is AB-308. In some embodiments, the anti-Tigit antibody is domvanalimab. In some embodiments, MCL-1 inhibitor is co-administered with the fusion protein. In some embodiments, the MCL-1 inhibitor is selected from GS-9716, 564315 (MIK665), AZD5991, AMG-176, AMG-397, ABBV-467, and PRT1419. In some embodiments, the anti-CD47 antibody is co-administered with the fusion protein. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments, the adenosine pathway inhibitor is co-administered with the fusion protein. In some embodiments, the adenosine pathway inhibitor is selected from an adenosine receptor antagonist, CD39 inhibitor, and CD73 inhibitor. In some embodiments, the adenosine receptor antagonist is a small molecule. In some embodiments, the adenosine receptor antagonist is selected from etrumadenant (AB729; GS-0928), M1069, taminadenant, TT-4, and TT-10. In some embodiments, the adenosine receptor antagonist is AB729 (etrumadenant). In some embodiments, the CD39 inhibitor is selected from TTX-030, IPH5201, SRF617, nucleotide derivatives, anthraquinone derivatives, and suramin derivatives. In some embodiments, the anthraquinone derivate is RB2. In some embodiments, the CD73 inhibitor is a small molecule. In some embodiments, CD73 inhibitor is selected from AB680 (quemliclustat), AK131, ATG-037, BMS-986179, mupadolimab, NZV930, oleclumab, ORIC-533, PT-199, and uliledlimab. In some embodiments, the CD73 inhibitor is AB680 (quemliclustat). In some embodiments, the anti-CCR8 antibody is co-administered with the fusion protein. In some embodiments, anti-CCR8 antibody causes depletion of regulatory T (Treg) cells. In some embodiments, the anti-CCR8 antibody is selected from BMS-986340, FPA157, HFB1011, HBM1022, IO-1, IPG276, JTX-1811 (GS-1811), LM-108, S-531011, and SRF-114. In some embodiments, the anti-CCR8 antibody is JTX-1811 (GS-1811). In some embodiments, the subject is a human subject. In some embodiments, the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 97% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 98% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence that is at least 99% identical to an amino acid sequence selected from the group consisting of SEQ ID NOs: 1-18 and 21-27 across the entire length of SEQ ID Nos: 1-18 and 21-27. In some embodiments, the fusion protein comprises an amino acid sequence selected from the group consisting of SEQ ID NOs: 1-18 and 21-27. In some embodiments, the fusion protein comprises an amino acid sequence that is at least about 97% identical to the amino acid sequence of SEQ ID NO: 14. In some embodiments, the fusion protein comprises an amino acid sequence that is at least about 98% identical to the amino acid sequence of SEQ ID NO: 14. In some embodiments, the fusion protein comprises an amino acid sequence that is at least about 99% identical to the amino acid sequence of SEQ ID NO: 14. In some embodiments, the fusion protein comprises the amino acid sequence of SEQ ID NO: 14.

Any of the methods disclosed herein may comprise co-administration of the fusion protein with one or more therapeutic agents. In some embodiments, the therapeutic agent is an anti-cancer agent, immunotherapy, immune checkpoint protein or receptor, inhibitor of an immune checkpoint protein or receptor, or an activator of an immune checkpoint protein or receptor disclosed herein.

In some embodiments, any of the methods disclosed herein further comprise co-administering the fusion protein with an anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, radiation therapy, or any combination thereof. In some embodiments, the fusion protein is administered prior to co-administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy. In some embodiments, the fusion protein is administered at least 1, 2, 3, 4, 5, 6, 7, 8, 9, or 10 days prior to administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy. In some embodiments, the fusion protein is administered after administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy. In some embodiments, the fusion protein is administered at least 1, 2, 3, 4, 5, 6, 7, 8, 9, or 10 days after administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy. In some embodiments, the fusion protein is administered concurrently with administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy. In some embodiments, the fusion protein is administered within 10, 15, 20, 25, 30, 35, 40, 45, 50, 55, 60, 65, 70, 75, 80, 85, 90, 95, 100, 110, 120, 130, 140, 150, 160, 170, 180, 190, or 200 minutes of administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy. In some embodiments, the fusion protein is administered within 1, 2, 3, 4, 5, 6, 7, 8, 9, 10, 11, 12, 13, 14, or 15 hours of administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy.

As used herein, the terms “inhibition of cancer” and “inhibition of cancer cell proliferation” refer to the inhibition of the growth, division, maturation or viability of cancer cells, and/or causing the death of cancer cells, individually or in aggregate with other cancer cells, by cytotoxicity, nutrient depletion, or the induction of apoptosis.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, “delaying” development of a disease or disorder, or one or more symptoms thereof, means to defer, hinder, slow, retard, stabilize and/or postpone development of the disease, disorder, or symptom thereof. This delay can be of varying lengths of time, depending on the history of the disease and/or subject being treated. As is evident to one skilled in the art, a sufficient or significant delay can, in effect, encompass prevention, in that the subject does not develop the disease, disorder, or symptom thereof. For example, a method that “delays” development of AIDS is a method that reduces the probability of disease development in a given time frame and/or reduces extent of the disease in a given time frame, when compared to not using the method. Such comparisons may be based on clinical studies, using a statistically significant number of subjects. For example, the development of AIDS can be detected using known methods, such as confirming a subject's HIV⁺ status and assessing the subject's T-cell count or other indication of AIDS development, such as extreme fatigue, weight loss, persistent diarrhea, high fever, swollen lymph nodes in the neck, armpits or groin, or presence of an opportunistic condition that is known to be associated with AIDS (e.g., a condition that is generally not present in subjects with functioning immune systems but does occur in AIDS patients). Development may also refer to disease progression that may be initially undetectable and includes occurrence, recurrence and onset.

As used herein, “prevention” or “preventing” refers to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject (e.g., administration of a therapeutic substance to a subject in the absence of detectable infectious agent (e.g., virus) in the subject). The subject may be an individual at risk of developing the disease or disorder, such as an individual who has one or more risk factors known to be associated with development or onset of the disease or disorder. For example, the term “preventing HIV infection” refers to administering to a subject who does not have a detectable HIV infection an anti-HIV therapeutic substance. It is understood that the subject for anti-HIV preventative therapy may be an individual at risk of contracting the HIV virus. Further, it is understood that prevention may not result in complete protection against onset of the disease or disorder. In some instances, prevention includes reducing the risk of developing the disease or disorder. The reduction of the risk may not result in complete elimination of the risk of developing the disease or disorder.

As used herein, an “anti-Trop2 ADC” comprises a monoclonal antibody and a topoisomerase I inhibitor. The topoisomerase I inhibitor may comprise irinotecan, topetecan, or SN-38. The anti-Trop2 ADC may comprise a structural formula of mAb-CL2A-SN-38, with a structure represented by:

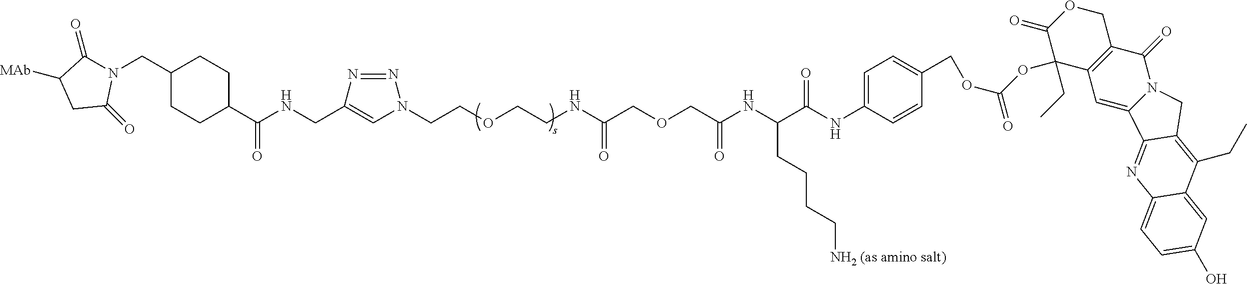

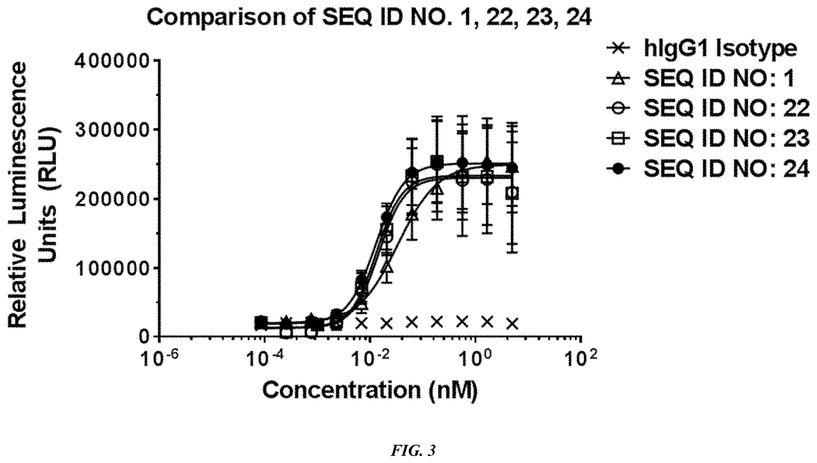

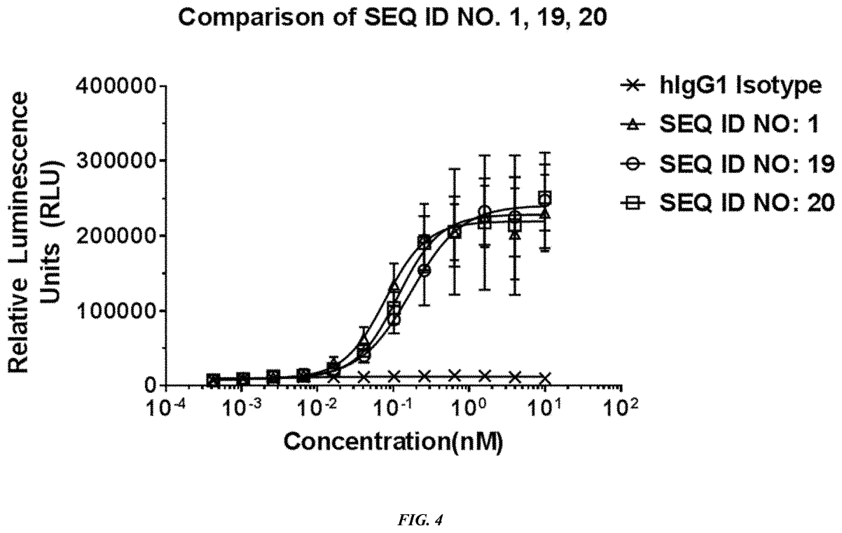

(described, e.g., in U.S. Pat. No. 7,999,083). The anti-Trop-2 ADC may comprise sacituzumab (hRS7; described, e.g., in WO2003074566, FIGS. 3 and 4). Examples of anti-Trop2 ADCs include, but are not limited to, sacituzumab govitecan, datopotamab deruxtecan (DS-1062), ESG-401, SKB-264, DAC-02 and BAT-8003.

As used herein, “sacituzumab govitecan- -hziy” and “sacituzumab govitecan” are used interchangeably and include any biosimilars thereof. Sacituzumab govitecan sold under the brand name TRODELVY® is an antibody-drug conjugate composed of 3 compounds: a humanized monoclonal antibody, a topoisomerase-I inhibitor, and linker protein. It binds to Trop-2 expressing cells, forming an internalized complex that releases SN-38 intracellularly.

With respect to subjects, in some embodiments, the methods of treatment provided herein may be used to treat a subject (e.g., human, monkey, dog, cat, mouse) who has been diagnosed with or is suspected of having cancer. In some embodiments, the methods of treatment provided herein can be used to treat a subject (e.g., human, monkey, dog, cat, mouse) who has been diagnosed with or is suspected of having a viral infection. As used herein, a subject refers to a mammal, including, for example, a human.

In some embodiments, the subject may be a human who exhibits one or more symptoms associated with cancer or hyperproliferative disease (e.g., a tumor). In some embodiments, the subject may be a human who exhibits one or more symptoms associated with cancer. Any of the methods of cancer treatment provided herein may be used to treat cancer at various stages. By way of example, the cancer stage includes but is not limited to early, advanced, locally advanced, remission, refractory, reoccurred after remission and progressive. In some embodiments, the subject is at an early stage of a cancer. In other embodiments, the subject is at an advanced stage of cancer. In various embodiments, the subject has a stage I, stage II, stage III or stage IV cancer. One or more administrations of the FLT3L-Fc fusion protein, optionally with one or more additional therapeutic agents, can promote reduction or retraction of a tumor, decrease or inhibit tumor growth or cancer cell proliferation, and/or induce, increase or promote tumor cell killing. In some embodiments, the subject is in cancer remission. One or more administrations of the FLT3L-Fc fusion protein, optionally with one or more additional therapeutic agents, can prevent or delay metastasis or recurrence of cancer.

In some embodiments, the subject may be a human who exhibits one or more symptoms associated with a viral infection (e.g., a detectable viral titer). In some embodiments, the subject may be a human who exhibits one or more symptoms associated with a viral infection. Any of the methods of antiviral treatment provided herein may be used to treat a viral infection at various stages. In some embodiments, the subject is at an early stage of a viral infection. In other embodiments, the subject is at an advanced stage of a viral infection. In some embodiments, one or more administrations of the FLT3L-Fc fusion protein, optionally with one or more additional therapeutic agents, can promote the reduction of a viral titer in a subject.

In some embodiments, the subject may be a human who is at risk, or genetically or otherwise predisposed (e.g., risk factor) to developing cancer or hyperproliferative disease who has or has not been diagnosed. In some embodiments, the subject may be a human who is at risk, or genetically or otherwise predisposed (e.g., risk factor) to a disease, disorder, or symptoms thereof, caused by a viral infection who has or has not been diagnosed.

As used herein, an “at risk” individual is an individual who is at risk of developing a condition to be treated. In some embodiments, an “at risk” subject is a subject who is at risk of developing cancer. Generally, an“at risk” subject may or may not have detectable disease, and may or may not have displayed detectable disease prior to the treatment methods described herein. “At risk” denotes that an individual has one or more so-called risk factors, which are measurable parameters that correlate with development of a disease or condition and are known in the art. For example, an at risk subject may have one or more risk factors, which are measurable parameters that correlate with development of cancer, which are described herein. A subject having one or more of these risk factors has a higher probability of developing cancer than an individual without these risk factor(s). In general, risk factors may include, for example, age, sex, race, diet, history of previous disease, presence of precursor disease, genetic (e.g., hereditary) considerations, and environmental exposure. In some embodiments, the subjects at risk for cancer include, for example, those having relatives who have experienced the disease, and those whose risk is determined by analysis of genetic or biochemical markers. In some embodiments, the at risk subject is at risk of developing symptoms of a viral infection. For example, individuals at risk for AIDS are those infected with HIV.

In addition, the subject may be a human who is undergoing one or more standard therapies, such as chemotherapy, radiotherapy, immunotherapy, surgery, or combination thereof. Accordingly, one or more kinase inhibitors may be administered before, during, or after administration of chemotherapy, radiotherapy, immunotherapy, surgery or combination thereof.

In certain embodiments, the subject may be a human who is (i) substantially refractory to at least one chemotherapy treatment, or (ii) is in relapse after treatment with chemotherapy, or both (i) and (ii). In some of embodiments, the subject is refractory to at least two, at least three, or at least four chemotherapy treatments (including standard or experimental chemotherapies).

The FLT3L-Fc fusion proteins described herein find use as a vaccine adjuvant, promoting, increasing, supplementing and/or boosting the immune response induced by the vaccine. In various embodiments, the vaccine can be an anticancer vaccine, antiviral vaccine, or antibacterial vaccine. In some embodiments, the anticancer vaccine is a neoantigen vaccine, wherein a neoantigen refers to a class of HLA-bound peptides that arise from tumor-specific mutations. Illustrative neoantigen anticancer vaccines are described, e.g., in Ott, et al., Nature. 2017 Jul. 13; 547(7662):217-221; Li, et al., Ann Oncol. 2017 Dec. 1; 28(suppl_12):xii11-xii17; Aldous, et al., Bioorg Med Chem. 2018 Jun. 1; 26(10):2842-2849; and Linette, et al, Trends Mol Med. 2017 October; 23(10):869-871. In various embodiments, the vaccine comprises an antiviral vaccine against a virus selected from the group consisting of hepatitis A virus (HAV), hepatitis B virus (HBV), human immunodeficiency virus (HIV), cytomegalovirus (CMV), a herpes simplex virus (HSV), Epstein-Barr virus (EBV), human orthopneumovirus or human respiratory syncytial virus (RSV), human papillomavirus (HPV), varicella-zoster virus, measles virus, mumps virus, poliovirus vaccine, influenza virus, paramyxovirus, rotavirus, Zika virus, Dengue virus and Ebola virus. In some embodiments, the vaccine comprises an antibacterial vaccine against a bacterium selected from the group consisting of Mycobacterium tuberculosis, pertussis, tetanus, diphtheria, meningococcus, pneumococcus, Haemophilus influenza, cholera, typhoid, and anthrax. Illustrative anticancer vaccines include without limitation Bacillus Calmettle-Guerin (TheraCys®)—a live attenuated strain of Mycobacterium bovis for non-muscle invasive bladder carcinoma; Sipuleucel-T (Provenge®)—a dendritic cell (DC) vaccine for metastatic castration resistant prostate cancer (mCRPC); talimogene laherparepvec (T-VEC or Imlygic®)—an oncolytic viral-based vaccine for advanced melanoma; and recombinant viral prostate cancer vaccine PROSTVAC®-VF/TRICOM™. In some embodiments, the anticancer vaccine is an antiviral vaccine. In some embodiments the anticancer vaccine is an HPV vaccine. In some embodiments, the HPV vaccine is PRGN-2009 (Precigen; PGEN Therapeutics). In some embodiments the HPV vaccine is Gardasil or Gardasil-9 (Merck&Co). In some embodiments, the HPV vaccine is Cervarix (GlaxoSmithKline Biologicals). In some embodiments the HSV vaccine is HSV529 (Sanofi Pasteur).

In some embodiments, the cell or population of cells that express FLT3 comprise dendritic cells (e.g., cDC1 cells and/or cDC2 cells), monocyte-derived dendritic cells (moDCs), and/or progenitor cells thereof. In some embodiments, the cell or population of cells that express FLT3 comprise hematopoietic progenitor cells. In some embodiments, the hematopoietic progenitor cells comprise Common Lymphoid Progenitors (CLPs), Early Progenitors with Lymphoid and Myeloid potential (EPLMs), granulocyte-monocyte (GM) progenitors (GMP), monocyte-derived dendritic cells (moDCs) progenitors, and/or early multi-potent progenitors (MPP) within the Lineage-kit+Sca1 (LSK) compartment. As appropriate, the cell can be contacted in vitro or in vivo. In some embodiments, conventional dendritic cells (e.g., cDC1 and/or cDC2) are expanded. In some embodiments, cDC1 dendritic cells (e.g., positive for surface expression of X-C motif chemokine receptor 1 (XCR1), thrombomodulin (THBD, CD141), and C-type lectin domain containing 9A (CLEC9A)) are expanded or induced to proliferate. In some embodiments, cDC2 dendritic cells (e.g., positive for surface expression of CD1c molecule (BDCA1)) are expanded or induced to proliferate. In some embodiments, dendritic cells positive for surface expression of BDCA1 (cDC1), BDCA2 (CLEC4c), BDCA3 (THBD) and/or BDCA4 (NRP1) are expanded or induced to proliferate. In some embodiments, the FLT3-expressing cells (e.g., dendritic cells) are expanded by at least about 10-fold, 20-fold, 50-fold, 100-fold, 200-fold, 300-fold, 400-fold, 500-fold, or more, e.g., in the tumor, in the lymph nodes, within 3 weeks of a single administration of the fusion protein, the homodimer, the heterodimer, the conjugate, the polynucleotide, the vector, the lipoplex (e.g., LNP) and/or the pharmaceutical composition.

As used herein, “HBV” refers to a virus described by NCBI Taxonomy ID: NCBI:txid10407.

As used herein, “HIV” refers to a virus described by NCBI Taxonomy ID: NCBI:txid11676.

As used herein, “SARS”-associated virus refers to a virus described by NCBI Taxonomy ID: NCBI:txid694009.

As used herein, “MERS”-associated virus refers to a virus described by NCBI Taxonomy ID: NCBI:txid1335626.

As used herein, “COVID-19-associated virus” or “SARS-CoV-2” refers to a virus described by NCBI Taxonomy ID: NCBI:txid2697049.

With respect to route of administration, in various embodiments, the FLT3L-Fc fusion protein, the homodimer, the heterodimer, the conjugate, the polynucleotide, the vector, the lipoplex, such as an LNP, and/or the pharmaceutical composition are administered systemically or locally. In some embodiments, the FLT3L-Fc fusion protein, the homodimer, the heterodimer, the conjugate, the polynucleotide, the vector, the lipoplex, such as an LNP, and/or the pharmaceutical composition can be administered intravenously, intratumorally, subcutaneously, intradermally, intramuscularly, intraperitoneally, intravesically, intracranially, intrathecally, intracavitary or intraventricularly. In embodiments involving combination therapies, as appropriate, the FLT3L-Fc fusion protein, the homodimer, the heterodimer, the conjugate, the polynucleotide, the vector, the lipoplex, such as an LNP, and/or the pharmaceutical composition and the one or more additional therapeutic agents can be administered by the same or different routes of administration. As appropriate, in certain embodiments, administration is via injection or infusion.

With respect to dosing, a therapeutically effective amount of FLT3L-Fc fusion protein, the homodimer, the heterodimer, the conjugate, the polynucleotide, the vector, the lipoplex, such as an LNP, and/or the pharmaceutical composition is administered to the subject. As used herein, a “therapeutically effective amount” means an amount sufficient to induce, promote and/or increase expansion and/or proliferation of FLT3⁺ cells, and thereby treat a subject (such as a human) suffering an indication, or to alleviate the existing symptoms of the indication (e.g., cancer, viral infection, bacterial infection). Determination of a therapeutically effective amount is within the capability of those skilled in the art, especially in light of the detailed disclosure provided herein.

In some embodiments, a therapeutically effective amount of a FLT3L-Fc fusion protein, homodimer, heterodimer, conjugate, polynucleotide, vector, lipoplex, such as an LNP, and/or pharmaceutical composition, as described herein, optionally, with one or more additional therapeutic agents, as described herein, can (i) reduce the number of diseased cells; (ii) reduce tumor size; (iii) inhibit, retard, slow to some extent, and preferably stop the diseased cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with cancer or myeloproliferative disease. In some embodiments, a therapeutically effective amount of a FLT3L-Fc fusion protein, homodimer, heterodimer, conjugate, polynucleotide, vector, lipoplex, such as an LNP, and/or pharmaceutical composition, as described herein, optionally, with one or more additional therapeutic agents, as described herein, can (i) reduce the number of cancer cells; (ii) reduce tumor size; (iii) inhibit, retard, slow to some extent, and preferably stop cancer cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with the cancer. In various embodiments, the amount is sufficient to ameliorate, palliate, lessen, and/or delay one or more of symptoms of cancer.

In some embodiments, a therapeutically effective amount of a FLT3L-Fc fusion protein, homodimer, heterodimer, conjugate, polynucleotide, vector, lipoplex, such as an LNP, and/or pharmaceutical composition, as described herein, optionally, with one or more additional therapeutic agents, as described herein, can inhibit the proliferation of a virus in a subject and/or delay to some extent one or more of the symptoms associated with viral infection (e.g., AIDS, SARS, MERS, liver disease caused by HBV). In various embodiments, the amount is sufficient to ameliorate, palliate, lessen, and/or delay one or more of symptoms of a viral infection.

An “increased” or “enhanced” amount (e.g., with respect to FLT3L+ cell expansion, antitumor response, cancer cell metastasis) refers to an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

A “decreased” or “reduced” or “lesser” amount (e.g., with respect to tumor size, cancer cell proliferation or growth) refers to a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least 200%, at least 500%, or at least 1000% of an amount or level described herein.

In some embodiments, the FLT3L-Fc fusion protein, homodimer, heterodimer, conjugate, polynucleotide, vector, lipoplex, such as an LNP, and/or pharmaceutical composition, as described herein, is administered at a dose in the range of about 0.5 μg/kg to about 5000 μg/kg, e.g., at least about 0.5 μg/kg per dose and up to about 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 15 μg/kg, 20 μg/kg, 30 μg/kg, 50 μg/kg, 100 μg/kg, 150 μg/kg, 300 μg/kg, 400 μg/kg, 500 μg/kg, 600 μg/kg, 700 μg/kg, 800 μg/kg, 900 μg/kg, 1000 μg/kg, 1500 μg/kg, 2000 μg/kg, 2500 μg/kg, 3000 μg/kg, 3500 μg/kg, 4000 μg/kg, or 5000 μg/kg, per dose. In some embodiments, the FLT3L-Fc fusion protein, homodimer, heterodimer, conjugate, polynucleotide, vector, lipoplex, such as an LNP, and/or pharmaceutical composition, as described herein, is administered at a dose in the range of about 1 μg/kg to about 100 μg/kg, per dose. In some embodiments, the FLT3L-Fc fusion protein, homodimer, heterodimer, conjugate, polynucleotide, vector, lipoplex, such as an LNP, and/or pharmaceutical composition, as described herein, is administered at a dose of 1 μg/kg, per dose. In some embodiments, the FLT3L-Fc fusion protein, homodimer, heterodimer, conjugate, polynucleotide, vector, lipoplex, such as an LNP, and/or pharmaceutical composition, as described herein, is administered at a dose of 3 μg/kg, per dose. In some embodiments, the FLT3L-Fc fusion protein, homodimer, heterodimer, conjugate, polynucleotide, vector, lipoplex, such as an LNP, and/or pharmaceutical composition, as described herein, is administered at a dose of 10 μg/kg, per dose. In some embodiments, the FLT3L-Fc fusion protein, homodimer, heterodimer, conjugate, polynucleotide, vector, lipoplex, such as an LNP, and/or pharmaceutical composition, as described herein, is administered at a dose of 30 μg/kg, per dose. In some embodiments, the FLT3L-Fc fusion protein, homodimer, heterodimer, conjugate, polynucleotide, vector, lipoplex, such as an LNP, and/or pharmaceutical composition, as described herein, is administered at a dose of 60 μg/kg, per dose. In some embodiments, the FLT3L-Fc fusion protein, homodimer, heterodimer, conjugate, polynucleotide, vector, lipoplex, such as an LNP, and/or pharmaceutical composition, as described herein, is administered at a dose of 100 μg/kg, per dose. In some embodiments, the FLT3L-Fc fusion protein, homodimer, heterodimer, conjugate, polynucleotide, vector, lipoplex, such as an LNP, and/or pharmaceutical composition, as described herein, is administered at a dose in the range of about 0.5 mg to about 50 mg, e.g., at least about 0.5 mg per dose and up to about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg or 50 mg per dose. In some embodiments, the FLT3L-Fc fusion protein, homodimer, heterodimer, conjugate, polynucleotide, vector, lipoplex, such as an LNP, and/or pharmaceutical composition, as described herein, is administered at a dose in the range of between about 600 μg to about 30000 μg, about 600 μg to about 29000 μg, about 600 μg to about 28000 μg, about 600 μg to about 27000 μg, about 600 μg to about 26000 μg, about 600 μg to about 25000 μg, about 600 μg to about 24000 μg, about 600 μg to about 23000 μg, about 600 μg to about 22000 μg, about 600 μg to about 21000 μg, about 600 μg to about 20000 μg, about 600 μg to about 19000 μg, about 600 μg to about 18000 μg, about 600 μg to about 17000 μg, about 600 μg to about 16000 μg, about 600 μg to about 15000 μg, about 600 μg to about 14000 μg, about 600 μg to about 13000 μg, about 600 μg to about 12000 μg, about 600 μg to about 11000 μg, about 600 μg to about 10000 μg, about 1000 μg to about 30000 μg, about 1000 μg to about 29000 μg, about 1000 μg to about 28000 μg, about 1000 μg to about 27000 μg, about 1000 μg to about 26000 μg, about 1000 μg to about 25000 μg, about 1000 μg to about 24000 μg, about 1000 μg to about 23000 μg, about 1000 μg to about 22000 μg, about 1000 μg to about 21000 μg, about 1000 μg to about 20000 μg, about 1000 μg to about 19000 μg, about 1000 μg to about 18000 μg, about 1000 μg to about 17000 μg, about 1000 μg to about 16000 μg, about 1000 μg to about 15000 μg, about 1000 μg to about 14000 μg, about 1000 μg to about 13000 μg, about 1000 μg to about 12000 μg, about 1000 μg to about 11000 μg, about 1000 μg to about 10000 μg, about 2000 μg to about 30000 μg, about 2000 μg to about 29000 μg, about 2000 μg to about 28000 μg, about 2000 μg to about 27000 μg, about 2000 μg to about 26000 μg, about 2000 μg to about 25000 μg, about 2000 μg to about 24000 μg, about 2000 μg to about 23000 μg, about 2000 μg to about 22000 μg, about 2000 μg to about 21000 μg, about 2000 μg to about 20000 μg, about 2000 μg to about 19000 μg, about 2000 μg to about 18000 μg, about 2000 μg to about 17000 μg, about 2000 μg to about 16000 μg, about 2000 μg to about 15000 μg, about 2000 μg to about 14000 μg, about 2000 μg to about 13000 μg, about 2000 μg to about 12000 μg, about 2000 μg to about 11000 μg, about 2000 μg to about 10000 μg per dose. In some embodiments, the FLT3L-Fc fusion protein, homodimer, heterodimer, conjugate, polynucleotide, vector, lipoplex, such as an LNP, and/or pharmaceutical composition, as described herein, is administered at a dose in the range of between about 200 μg to about 3000 μg. In some embodiments, the FLT3L-Fc fusion protein, homodimer, heterodimer, conjugate, polynucleotide, vector, lipoplex, such as an LNP, and/or pharmaceutical composition, as described herein, is administered at a dose in the range of between about 200 μg to about 2500 μg. In some embodiments, the FLT3L-Fc fusion protein, homodimer, heterodimer, conjugate, polynucleotide, vector, lipoplex, such as an LNP, and/or pharmaceutical composition, as described herein, is administered at a dose in the range of between about 200 μg to about 2000 μg. In some embodiments, the FLT3L-Fc fusion protein, homodimer, heterodimer, conjugate, polynucleotide, vector, lipoplex, such as an LNP, and/or pharmaceutical composition, as described herein, is administered at a dose of up to about 30000 μg, 29000 μg, 28000 μg, 27000 μg, 26000 μg, 25000 μg, 24000 μg, 23000 μg, 22000 μg, 21000 μg, 20000 μg, 19000 μg, 18000 μg, 17000 μg, 16000 μg, 15000 μg, 14000 μg, 13000 μg, 12000 μg, 11000 μg, 10000 μg, 9000 μg, 8000 μg, 7000 μg, 6000 μg, or 5000 μg per dose. In some embodiments, the FLT3L-Fc fusion protein, homodimer, heterodimer, conjugate, polynucleotide, vector, lipoplex, such as an LNP, and/or pharmaceutical composition, as described herein, is administered at a dose of up to about 20000 μg. In some embodiments, the FLT3L-Fc fusion protein, homodimer, heterodimer, conjugate, polynucleotide, vector, lipoplex, such as an LNP, and/or pharmaceutical composition, as described herein, is administered at a dose of up to about 10000 μg. In some embodiments, the FLT3L-Fc fusion protein, homodimer, heterodimer, conjugate, polynucleotide, vector, lipoplex, such as an LNP, and/or pharmaceutical composition, as described herein, is administered at a dose of up to about 5000 μg. In some embodiments, the FLT3L-Fc fusion protein, homodimer, heterodimer, conjugate, polynucleotide, vector, lipoplex, such as an LNP, and/or pharmaceutical composition, as described herein, is administered at a dose of up to about 3000 μg. In some embodiments, the FLT3L-Fc fusion protein, homodimer, heterodimer, conjugate, polynucleotide, vector, lipoplex, such as an LNP, and/or pharmaceutical composition, as described herein, is administered at a dose of up to about 2500 μg. In some embodiments, the FLT3L-Fc fusion protein, homodimer, heterodimer, conjugate, polynucleotide, vector, lipoplex, such as an LNP, and/or pharmaceutical composition, as described herein, is administered at a dose of up to about 2000 μg. In some embodiments, the FLT3L-Fc fusion protein, homodimer, heterodimer, conjugate, polynucleotide, vector, lipoplex, such as an LNP, and/or pharmaceutical composition, as described herein, is administered at a dose of at least about 225 μg, 250 μg, 275 μg, 300 μg, 400 μg, 500 μg, 600 μg, 625 μg, 650 μg, 675 μg, 700 μg, 800 μg, 900 μg, 1000 μg, 1100 μg, 1200 μg, 1300 μg, 1400 μg, 1500 μg, 1600 μg, 1700 μg, 1800 μg, 1900 μg, 2000 μg, 2100 μg, 2200 μg, 2300 μg, 2400 μg, 2500 μg, 2600 μg, 2700 μg, 2800 μg, 2900 μg, or 3000 μg per dose. In some embodiments, for any of the methods disclosed herein, at least about 800 μg of the fusion protein is administered to the subject per dose. In some embodiments, for any of the methods disclosed herein, at least about 1000 μg of the fusion protein is administered to the subject per dose. In some embodiments, for any of the methods disclosed herein, at least about 1500 μg of the fusion protein is administered to the subject per dose. In some embodiments, for any of the methods disclosed herein, at least about 2000 μg of the fusion protein is administered to the subject per dose. In some embodiments, for any of the methods disclosed herein, at least about 2500 μg of the fusion protein is administered to the subject per dose. In some embodiments, for any of the methods disclosed herein, at least about 3000 μg of the fusion protein is administered to the subject per dose. In some embodiments, the FLT3L-Fc fusion protein, homodimer, heterodimer, conjugate, polynucleotide, vector, lipoplex, such as an LNP, and/or pharmaceutical composition, as described herein, is administered at a dose that saturates FLT3 receptors in the tumor. In some embodiments, the FLT3L-Fc fusion protein, homodimer, heterodimer, conjugate, polynucleotide, vector, lipoplex, such as an LNP, and/or pharmaceutical composition, as described herein, is administered at a dose that saturates FLT3 receptors in the subject.

With respect to scheduling of administrations, in various embodiments, the methods comprise administering multiple administrations or doses of the fusion protein, the homodimer, the heterodimer, the conjugate, the polynucleotide, the vector, the lipoplex, such as an LNP, and/or the pharmaceutical composition, optionally with one or more additional therapeutic agents, at predetermined intervals. As appropriate, in various embodiments, the FLT3L-Fc fusion protein, the homodimer, the heterodimer, the conjugate, the polynucleotide, the vector, the lipoplex, such as an LNP, and/or the pharmaceutical composition can be administered once weekly (i.e., QW), once bi-weekly (i.e., once every other week, or once every two weeks or Q2W), once thrice-weekly (i.e., once every three weeks or Q3W), once monthly (i.e., QM) or once bi-monthly dosing (i.e., once every other month, or once every two months or Q2M), or less often. In some embodiments, for any of the methods disclosed herein, at least two doses of the fusion protein, the homodimer, the heterodimer, the conjugate, the polynucleotide, the vector, the lipoplex, such as an LNP, and/or the pharmaceutical composition are administered at least 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days apart. In some embodiments, for any of the methods disclosed herein, at least two doses of the fusion protein, the homodimer, the heterodimer, the conjugate, the polynucleotide, the vector, the lipoplex, such as an LNP, and/or the pharmaceutical composition are administered at least 10 days apart. In some embodiments, for any of the methods disclosed herein, at least two doses of the fusion protein, the homodimer, the heterodimer, the conjugate, the polynucleotide, the vector, the lipoplex, such as an LNP, and/or the pharmaceutical composition are administered at least 14 days apart. In some embodiments, for any of the methods disclosed herein, at least two doses of the fusion protein, the homodimer, the heterodimer, the conjugate, the polynucleotide, the vector, the lipoplex, such as an LNP, and/or the pharmaceutical composition are administered at least 21 days apart. In some embodiments, for any of the methods disclosed herein, at least two doses of the fusion protein, the homodimer, the heterodimer, the conjugate, the polynucleotide, the vector, the lipoplex, such as an LNP, and/or the pharmaceutical composition are administered at least 28 days apart. In some embodiments, for any of the methods disclosed herein, (i) at least two doses of the fusion protein, the homodimer, the heterodimer, the conjugate, the polynucleotide, the vector, the lipoplex, such as an LNP, and/or the pharmaceutical composition are administered at least 10 days apart; and (ii) at least two additional doses of the fusion protein, the homodimer, the heterodimer, the conjugate, the polynucleotide, the vector, the lipoplex, such as an LNP, and/or the pharmaceutical composition are administered at least 21 days apart. In some embodiments, for any of the methods disclosed herein, (i) at least two doses of the fusion protein, the homodimer, the heterodimer, the conjugate, the polynucleotide, the vector, the lipoplex, such as an LNP, and/or the pharmaceutical composition are administered at least 14 days apart; and (ii) at least two additional doses of the fusion protein, the homodimer, the heterodimer, the conjugate, the polynucleotide, the vector, the lipoplex, such as an LNP, and/or the pharmaceutical composition are administered at least 21 days apart. In some embodiments, for any of the methods disclosed herein, (i) at least two doses of the fusion protein, the homodimer, the heterodimer, the conjugate, the polynucleotide, the vector, the lipoplex, such as an LNP, and/or the pharmaceutical composition are administered at least 14 days apart; and (ii) at least two additional doses of the fusion protein, the homodimer, the heterodimer, the conjugate, the polynucleotide, the vector, the lipoplex, such as an LNP, and/or the pharmaceutical composition are administered at least 28 days apart. In some embodiments, for any of the methods disclosed herein, at least two doses of the fusion protein, the homodimer, the heterodimer, the conjugate, the polynucleotide, the vector, the lipoplex, such as an LNP, and/or the pharmaceutical composition are administered at least 1, 2, 3, 4, 5, 6, 7, 8, 9, 10, 11, 12, 13, 14, 15, 16, 17, 18, 19, or 20 weeks apart. In some embodiments, for any of the methods disclosed herein, at least two doses of the fusion protein, the homodimer, the heterodimer, the conjugate, the polynucleotide, the vector, the lipoplex, such as an LNP, and/or the pharmaceutical composition are administered at least 1 week apart. In some embodiments, for any of the methods disclosed herein, at least two doses of the fusion protein, the homodimer, the heterodimer, the conjugate, the polynucleotide, the vector, the lipoplex, such as an LNP, and/or the pharmaceutical composition are administered at least 2 weeks apart. In some embodiments, for any of the methods disclosed herein, at least two doses of the fusion protein, the homodimer, the heterodimer, the conjugate, the polynucleotide, the vector, the lipoplex, such as an LNP, and/or the pharmaceutical composition are administered at least 3 weeks apart. In some embodiments, for any of the methods disclosed herein, at least two doses of the fusion protein, the homodimer, the heterodimer, the conjugate, the polynucleotide, the vector, the lipoplex, such as an LNP, and/or the pharmaceutical composition are administered at least 4 weeks apart. In some embodiments, for any of the methods disclosed herein, (i) at least two doses of the fusion protein, the homodimer, the heterodimer, the conjugate, the polynucleotide, the vector, the lipoplex, such as an LNP, and/or the pharmaceutical composition are administered at least 1 week apart; and (ii) at least two additional doses of the fusion protein, the homodimer, the heterodimer, the conjugate, the polynucleotide, the vector, the lipoplex, such as an LNP, and/or the pharmaceutical composition are administered at least 3 weeks apart. In some embodiments, for any of the methods disclosed herein, (i) at least two doses of the fusion protein, the homodimer, the heterodimer, the conjugate, the polynucleotide, the vector, the lipoplex, such as an LNP, and/or the pharmaceutical composition are administered at least 2 weeks apart; and (ii) at least two additional doses of the fusion protein, the homodimer, the heterodimer, the conjugate, the polynucleotide, the vector, the lipoplex, such as an LNP, and/or the pharmaceutical composition are administered at least 3 weeks apart. In some embodiments, for any of the methods disclosed herein, (i) at least two doses of the fusion protein, the homodimer, the heterodimer, the conjugate, the polynucleotide, the vector, the lipoplex, such as an LNP, and/or the pharmaceutical composition are administered at least 2 weeks apart; and (ii) at least two additional doses of the fusion protein, the homodimer, the heterodimer, the conjugate, the polynucleotide, the vector, the lipoplex, such as an LNP, and/or the pharmaceutical composition are administered at least 4 weeks apart. As appropriate, the fusion protein, the homodimer, the heterodimer, the conjugate, the polynucleotide, the vector, the lipoplex, such as an LNP, and/or the pharmaceutical composition and the one or more additional therapeutic agents can be co-administered according to the same schedule (e.g., co-administered at the same time intervals) or different schedules (e.g., co-administered at different time intervals). in various embodiments, the FLT3L-Fc fusion protein, the homodimer, the heterodimer, the conjugate, the polynucleotide, the vector, the lipoplex, such as an LNP, and/or the pharmaceutical composition can be administered first, followed by administration of the one or more additional therapeutic agents, e.g., 1, 2 or 3 weeks later, e.g., after detectable or sufficient expansion of FLT3-expressing cells, e.g., cDC1 dendritic cells.

In some embodiments, for any of the methods disclosed herein, the method further comprises pausing administration of the fusion protein, the homodimer, the heterodimer, the conjugate, the polynucleotide, the vector, the lipoplex, such as an LNP, and/or the pharmaceutical composition for at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 weeks. In some embodiments, for any of the methods disclosed herein, administration of the fusion protein, the homodimer, the heterodimer, the conjugate, the polynucleotide, the vector, the lipoplex, such as an LNP, and/or the pharmaceutical composition is paused for at least about 8 weeks. In some embodiments, for any of the methods disclosed herein, administration of the fusion protein, the homodimer, the heterodimer, the conjugate, the polynucleotide, the vector, the lipoplex, such as an LNP, and/or the pharmaceutical composition is paused for at least about 10 weeks. In some embodiments, for any of the methods disclosed herein, administration of the fusion protein, the homodimer, the heterodimer, the conjugate, the polynucleotide, the vector, the lipoplex, such as an LNP, and/or the pharmaceutical composition is paused for at least about 12 weeks. In some embodiments, for any of the methods disclosed herein, administration of the fusion protein, the homodimer, the heterodimer, the conjugate, the polynucleotide, the vector, the lipoplex, such as an LNP, and/or the pharmaceutical composition is paused for at least about 14 weeks. In some embodiments, for any of the methods disclosed herein, administration of the fusion protein, the homodimer, the heterodimer, the conjugate, the polynucleotide, the vector, the lipoplex, such as an LNP, and/or the pharmaceutical composition is paused for at least about 16 weeks. In some embodiments, for any of the methods disclosed herein, administration of the fusion protein, the homodimer, the heterodimer, the conjugate, the polynucleotide, the vector, the lipoplex, such as an LNP, and/or the pharmaceutical composition is paused for at least about 18 weeks. In some embodiments, for any of the methods disclosed herein, administration of the fusion protein, the homodimer, the heterodimer, the conjugate, the polynucleotide, the vector, the lipoplex, such as an LNP, and/or the pharmaceutical composition is paused for at least about 20 weeks. In some embodiments, for any of the methods disclosed herein, the method further comprises pausing administration of the the fusion protein, the homodimer, the heterodimer, the conjugate, the polynucleotide, the vector, the lipoplex, such as an LNP, and/or the pharmaceutical composition for at least about 1, 2, 3, 4, 5, 6, 7, 8, 9, 10, 11, or 12 months. In some embodiments, for any of the methods disclosed herein, administration of the fusion protein, the homodimer, the heterodimer, the conjugate, the polynucleotide, the vector, the lipoplex, such as an LNP, and/or the pharmaceutical composition is paused for at least about 2 months. In some embodiments, for any of the methods disclosed herein, administration of the fusion protein, the homodimer, the heterodimer, the conjugate, the polynucleotide, the vector, the lipoplex, such as an LNP, and/or the pharmaceutical composition is paused for at least about 3 months. In some embodiments, for any of the methods disclosed herein, administration of the fusion protein, the homodimer, the heterodimer, the conjugate, the polynucleotide, the vector, the lipoplex, such as an LNP, and/or the pharmaceutical composition is paused for at least about 4 months. In some embodiments, for any of the methods disclosed herein, administration of the fusion protein, the homodimer, the heterodimer, the conjugate, the polynucleotide, the vector, the lipoplex, such as an LNP, and/or the pharmaceutical composition is paused for at least about 5 months. In some embodiments, for any of the methods disclosed herein, administration of the fusion protein, the homodimer, the heterodimer, the conjugate, the polynucleotide, the vector, the lipoplex, such as an LNP, and/or the pharmaceutical composition is paused for at least about 6 months.

In some embodiments, for any of the methods disclosed herein, at least about 2, 3, 4, 5, 6, 7, 8, 9, 10, 11, 12, 13, 14, 15, 16, 17, 18, 19, or 20 doses of the fusion protein, the homodimer, the heterodimer, the conjugate, the polynucleotide, the vector, the lipoplex, such as an LNP, and/or the pharmaceutical composition are administered to the subject prior to pausing administration of the fusion protein, the homodimer, the heterodimer, the conjugate, the polynucleotide, the vector, the lipoplex, such as an LNP, and/or the pharmaceutical composition. In some embodiments, for any of the methods disclosed herein, at least about 3 doses of the fusion protein, the homodimer, the heterodimer, the conjugate, the polynucleotide, the vector, the lipoplex, such as an LNP, and/or the pharmaceutical composition are administered to the subject prior to pausing administration of the fusion protein, the homodimer, the heterodimer, the conjugate, the polynucleotide, the vector, the lipoplex, such as an LNP, and/or the pharmaceutical composition. In some embodiments, for any of the methods disclosed herein, at least about 4 doses of the fusion protein, the homodimer, the heterodimer, the conjugate, the polynucleotide, the vector, the lipoplex, such as an LNP, and/or the pharmaceutical composition are administered to the subject prior to pausing administration of the fusion protein, the homodimer, the heterodimer, the conjugate, the polynucleotide, the vector, the lipoplex, such as an LNP, and/or the pharmaceutical composition. In some embodiments, for any of the methods disclosed herein, at least about 5 doses of the fusion protein, the homodimer, the heterodimer, the conjugate, the polynucleotide, the vector, the lipoplex, such as an LNP, and/or the pharmaceutical composition are administered to the subject prior to pausing administration of the fusion protein, the homodimer, the heterodimer, the conjugate, the polynucleotide, the vector, the lipoplex, such as an LNP, and/or the pharmaceutical composition. In some embodiments, for any of the methods disclosed herein, at least about 6 doses of the fusion protein, the homodimer, the heterodimer, the conjugate, the polynucleotide, the vector, the lipoplex, such as an LNP, and/or the pharmaceutical composition are administered to the subject prior to pausing administration of the fusion protein, the homodimer, the heterodimer, the conjugate, the polynucleotide, the vector, the lipoplex, such as an LNP, and/or the pharmaceutical composition. In some embodiments, for any of the methods disclosed herein, at least about 7 doses of the fusion protein, the homodimer, the heterodimer, the conjugate, the polynucleotide, the vector, the lipoplex, such as an LNP, and/or the pharmaceutical composition are administered to the subject prior to pausing administration of the fusion protein, the homodimer, the heterodimer, the conjugate, the polynucleotide, the vector, the lipoplex, such as an LNP, and/or the pharmaceutical composition. In some embodiments, for any of the methods disclosed herein, at least about 8 doses of the fusion protein, the homodimer, the heterodimer, the conjugate, the polynucleotide, the vector, the lipoplex, such as an LNP, and/or the pharmaceutical composition are administered to the subject prior to pausing administration of the fusion protein, the homodimer, the heterodimer, the conjugate, the polynucleotide, the vector, the lipoplex, such as an LNP, and/or the pharmaceutical composition. In some embodiments, for any of the methods disclosed herein, less than about 20, 19, 18, 17, 16, 15, 14, 13, 12, 11, 10, or 9 doses of the fusion protein, the homodimer, the heterodimer, the conjugate, the polynucleotide, the vector, the lipoplex, such as an LNP, and/or the pharmaceutical composition are administered to the subject prior to pausing administration of the fusion protein, the homodimer, the heterodimer, the conjugate, the polynucleotide, the vector, the lipoplex, such as an LNP, and/or the pharmaceutical composition. In some embodiments, for any of the methods disclosed herein, less than about 12 doses of the fusion protein, the homodimer, the heterodimer, the conjugate, the polynucleotide, the vector, the lipoplex, such as an LNP, and/or the pharmaceutical composition are administered to the subject prior to pausing administration of the fusion protein, the homodimer, the heterodimer, the conjugate, the polynucleotide, the vector, the lipoplex, such as an LNP, and/or the pharmaceutical composition. In some embodiments, for any of the methods disclosed herein, less than about 10 doses of the fusion protein, the homodimer, the heterodimer, the conjugate, the polynucleotide, the vector, the lipoplex, such as an LNP, and/or the pharmaceutical composition are administered to the subject prior to pausing administration of the fusion protein, the homodimer, the heterodimer, the conjugate, the polynucleotide, the vector, the lipoplex, such as an LNP, and/or the pharmaceutical composition. In some embodiments, for any of the methods disclosed herein, less than about 8 doses of the fusion protein, the homodimer, the heterodimer, the conjugate, the polynucleotide, the vector, the lipoplex, such as an LNP, and/or the pharmaceutical composition are administered to the subject prior to pausing administration of the fusion protein, the homodimer, the heterodimer, the conjugate, the polynucleotide, the vector, the lipoplex, such as an LNP, and/or the pharmaceutical composition. In some embodiments, for any of the methods disclosed herein, about 2 to about 15, about 2 to about 12, about 2 to about 10, about 2 to about 8, about 3 to about 15, about 3 to about 12, about 3 to about 10, about 3 to about 8, about 4 to about 15, about 4 to about 12, about 4 to about 10, about 4 to about 8, about 5 to about 15, about 5 to about 12, about 5 to about 10, about 5 to about 8, about 6 to about 15, about 6 to about 12, about 6 to about 10, about 6 to about 8 doses of the fusion protein, the homodimer, the heterodimer, the conjugate, the polynucleotide, the vector, the lipoplex, such as an LNP, and/or the pharmaceutical composition are administered to the subject prior to pausing administration of the fusion protein, the homodimer, the heterodimer, the conjugate, the polynucleotide, the vector, the lipoplex, such as an LNP, and/or the pharmaceutical composition. In some embodiments, for any of the methods disclosed herein, about 2 to about 10 doses of the fusion protein, the homodimer, the heterodimer, the conjugate, the polynucleotide, the vector, the lipoplex, such as an LNP, and/or the pharmaceutical composition are administered to the subject prior to pausing administration of the fusion protein, the homodimer, the heterodimer, the conjugate, the polynucleotide, the vector, the lipoplex, such as an LNP, and/or the pharmaceutical composition. In some embodiments, for any of the methods disclosed herein, about 3 to about 12 doses of the fusion protein, the homodimer, the heterodimer, the conjugate, the polynucleotide, the vector, the lipoplex, such as an LNP, and/or the pharmaceutical composition are administered to the subject prior to pausing administration of the fusion protein, the homodimer, the heterodimer, the conjugate, the polynucleotide, the vector, the lipoplex, such as an LNP, and/or the pharmaceutical composition. In some embodiments, for any of the methods disclosed herein, about 3 to about 9 doses of the fusion protein, the homodimer, the heterodimer, the conjugate, the polynucleotide, the vector, the lipoplex, such as an LNP, and/or the pharmaceutical composition are administered to the subject prior to pausing administration of the fusion protein, the homodimer, the heterodimer, the conjugate, the polynucleotide, the vector, the lipoplex, such as an LNP, and/or the pharmaceutical composition. In some embodiments, for any of the methods disclosed herein, about 4 to about 12 doses of the fusion protein, the homodimer, the heterodimer, the conjugate, the polynucleotide, the vector, the lipoplex, such as an LNP, and/or the pharmaceutical composition are administered to the subject prior to pausing administration of the fusion protein, the homodimer, the heterodimer, the conjugate, the polynucleotide, the vector, the lipoplex, such as an LNP, and/or the pharmaceutical composition. In some embodiments, for any of the methods disclosed herein, about 4 to about 9 doses of the fusion protein, the homodimer, the heterodimer, the conjugate, the polynucleotide, the vector, the lipoplex, such as an LNP, and/or the pharmaceutical composition are administered to the subject prior to pausing administration of the fusion protein, the homodimer, the heterodimer, the conjugate, the polynucleotide, the vector, the lipoplex, such as an LNP, and/or the pharmaceutical composition.

In some embodiments, for any of the methods disclosed herein, a plurality of doses of the fusion protein, the homodimer, the heterodimer, the conjugate, the polynucleotide, the vector, the lipoplex, such as an LNP, and/or the pharmaceutical composition is administered over a duration of at least about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks. In some embodiments, for any of the methods disclosed herein, the plurality of doses of the fusion protein, the homodimer, the heterodimer, the conjugate, the polynucleotide, the vector, the lipoplex, such as an LNP, and/or the pharmaceutical composition is administered over a duration of at least about 6 weeks. In some embodiments, for any of the methods disclosed herein, the plurality of doses of the fusion protein, the homodimer, the heterodimer, the conjugate, the polynucleotide, the vector, the lipoplex, such as an LNP, and/or the pharmaceutical composition is administered over a duration of at least about 8 weeks. In some embodiments, for any of the methods disclosed herein, the plurality of doses of the fusion protein, the homodimer, the heterodimer, the conjugate, the polynucleotide, the vector, the lipoplex, such as an LNP, and/or the pharmaceutical composition is administered over a duration of at least about 10 weeks. In some embodiments, for any of the methods disclosed herein, the plurality of doses of the fusion protein, the homodimer, the heterodimer, the conjugate, the polynucleotide, the vector, the lipoplex, such as an LNP, and/or the pharmaceutical composition is administered over a duration of at least about 14 weeks. In some embodiments, for any of the methods disclosed herein, the plurality of doses of the fusion protein, the homodimer, the heterodimer, the conjugate, the polynucleotide, the vector, the lipoplex, such as an LNP, and/or the pharmaceutical composition is administered over a duration of at least about 18 weeks. In some embodiments, for any of the methods disclosed herein, the plurality of doses of the fusion protein, the homodimer, the heterodimer, the conjugate, the polynucleotide, the vector, the lipoplex, such as an LNP, and/or the pharmaceutical composition is administered over a duration of at least about 20 weeks. In some embodiments, for any of the methods disclosed herein, the plurality of doses of the fusion protein, the homodimer, the heterodimer, the conjugate, the polynucleotide, the vector, the lipoplex, such as an LNP, and/or the pharmaceutical composition is administered over a duration of at least about 30 weeks. In some embodiments, for any of the methods disclosed herein, a plurality of doses of the fusion protein, the homodimer, the heterodimer, the conjugate, the polynucleotide, the vector, the lipoplex, such as an LNP, and/or the pharmaceutical composition is administered over a duration of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 months. In some embodiments, for any of the methods disclosed herein, the plurality of doses of the fusion protein, the homodimer, the heterodimer, the conjugate, the polynucleotide, the vector, the lipoplex, such as an LNP, and/or the pharmaceutical composition is administered over a duration of at least about 2 months. In some embodiments, for any of the methods disclosed herein, the plurality of doses of the fusion protein, the homodimer, the heterodimer, the conjugate, the polynucleotide, the vector, the lipoplex, such as an LNP, and/or the pharmaceutical composition is administered over a duration of at least about 3 months. In some embodiments, for any of the methods disclosed herein, the plurality of doses of the fusion protein, the homodimer, the heterodimer, the conjugate, the polynucleotide, the vector, the lipoplex, such as an LNP, and/or the pharmaceutical composition is administered over a duration of at least about 4 months. In some embodiments, for any of the methods disclosed herein, the plurality of doses of the fusion protein, the homodimer, the heterodimer, the conjugate, the polynucleotide, the vector, the lipoplex, such as an LNP, and/or the pharmaceutical composition is administered over a duration of at least about 6 months. In some embodiments, for any of the methods disclosed herein, the plurality of doses of the fusion protein, the homodimer, the heterodimer, the conjugate, the polynucleotide, the vector, the lipoplex, such as an LNP, and/or the pharmaceutical composition is administered over a duration of at least about 8 months. In some embodiments, for any of the methods disclosed herein, the plurality of doses of the fusion protein, the homodimer, the heterodimer, the conjugate, the polynucleotide, the vector, the lipoplex, such as an LNP, and/or the pharmaceutical composition is administered over a duration of at least about 10 months. In some embodiments, for any of the methods disclosed herein, the plurality of doses of the fusion protein, the homodimer, the heterodimer, the conjugate, the polynucleotide, the vector, the lipoplex, such as an LNP, and/or the pharmaceutical composition is administered over a duration of at least about 12 months. In some embodiments, for any of the methods disclosed herein, the plurality of doses of the fusion protein, the homodimer, the heterodimer, the conjugate, the polynucleotide, the vector, the lipoplex, such as an LNP, and/or the pharmaceutical composition is administered over a duration of at least about 14 months.

Examples of tissues containing cancerous cells whose proliferation is inhibited by the FLT3L-Fc fusion proteins described herein and against which the methods described herein are useful include but are not limited to breast, prostate, brain, blood, bone marrow, liver, pancreas, skin, kidney, colon, ovary, lung, testicle, penis, thyroid, parathyroid, pituitary, thymus, retina, uvea, conjunctiva, spleen, head, neck, trachea, gall bladder, rectum, salivary gland, adrenal gland, throat, esophagus, lymph nodes, sweat glands, sebaceous glands, muscle, heart, and stomach.

In some embodiments, the subject has a solid tumor. In various embodiments, the cancer or tumor is malignant and/or a metastatic. In various embodiments, the subject has a cancer selected from the group consisting of an epithelial tumor (e.g., a carcinoma, a squamous cell carcinoma, a basal cell carcinoma, a squamous intraepithelial neoplasia), a glandular tumor (e.g., an adenocarcinoma, an adenoma, an adenomyoma), a mesenchymal or soft tissue tumor (e.g., a sarcoma, a rhabdomyosarcoma, a leiomyosarcoma, a liposarcoma, a fibrosarcoma, a dermatofibrosarcoma, a neurofibrosarcoma, a fibrous histiocytoma, an angiosarcoma, an angiomyxoma, a leiomyoma, a chondroma, a chondrosarcoma, an alveolar soft-part sarcoma, an epithelioid hemangioendothelioma, a Spitz tumor, a synovial sarcoma), and a lymphoma.

In various embodiments, the subject has a solid tumor in or arising from a tissue or organ selected from the group consisting of:
bone (e.g., adamantinoma, aneurysmal bone cysts, angiosarcoma, chondroblastoma, chondroma, chondromyxoid fibroma, chondrosarcoma, chordoma, dedifferentiated chondrosarcoma, enchondroma, epithelioid hemangioendothelioma, fibrous dysplasia of the bone, giant cell tumour of bone, haemangiomas and related lesions, osteoblastoma, osteochondroma, osteosarcoma, osteoid osteoma, osteoma, periosteal chondroma, Desmoid tumor, Ewing sarcoma);
lips and oral cavity (e.g., odontogenic ameloblastoma, oral leukoplakia, oral squamous cell carcinoma, primary oral mucosal melanoma); salivary glands (e.g., pleomorphic salivary gland adenoma, salivary gland adenoid cystic carcinoma, salivary gland mucoepidermoid carcinoma, salivary gland Warthin's tumors);
esophagus (e.g., Barrett's esophagus, dysplasia and adenocarcinoma);
gastrointestinal tract, including stomach (e.g., gastric adenocarcinoma, primary gastric lymphoma, gastrointestinal stromal tumors (GISTs), metastatic deposits, gastric carcinoids, gastric sarcomas, neuroendocrine carcinoma, gastric primary squamous cell carcinoma, gastric adenoacanthomas), intestines and smooth muscle (e.g., intravenous leiomyomatosis), colon (e.g., colorectal adenocarcinoma), rectum, anus;
pancreas (e.g., serous neoplasms, including microcystic or macrocystic serous cystadenoma, solid serous cystadenoma, Von Hippel-Landau (VHL)-associated serous cystic neoplasm, serous cystadenocarcinoma; mucinous cystic neoplasms (MCN), intraductal papillary mucinous neoplasms (IPMN), intraductal oncocytic papillary neoplasms (IOPN), intraductal tubular neoplasms, cystic acinar neoplasms, including acinar cell cystadenoma, acinar cell cystadenocarcinoma, pancreatic adenocarcinoma, invasive pancreatic ductal adenocarcinomas, including tubular adenocarcinoma, adenosquamous carcinoma, colloid carcinoma, medullary carcinoma, hepatoid carcinoma, signet ring cell carcinoma, undifferentiated carcinoma, undifferentiated carcinoma with osteoclast-like giant cells, acinar cell carcinoma, neuroendocrine neoplasms, neuroendocrine microadenoma, neuroendocrine tumors (NET), neuroendocrine carcinoma (NEC), including small cell or large cell NEC, insulinoma, gastrinoma, glucagonoma, serotonin-producing NET, somatostatinoma, VIPoma, solid-pseudopapillary neoplasms (SPN), pancreatoblastoma);
gall bladder (e.g., carcinoma of the gallbladder and extrahepatic bile ducts, intrahepatic cholangiocarcinoma);
neuro-endocrine (e.g., adrenal cortical carcinoma, carcinoid tumors, phaeochromocytoma, pituitary adenomas);
thyroid (e.g., anaplastic (undifferentiated) carcinoma, medullary carcinoma, oncocytic tumors, papillary carcinoma, adenocarcinoma);
liver (e.g., adenoma, combined hepatocellular and cholangiocarcinoma, fibrolamellar carcinoma, hepatoblastoma, hepatocellular carcinoma, mesenchymal, nested stromal epithelial tumor, undifferentiated carcinoma; hepatocellular carcinoma, intrahepatic cholangiocarcinoma, bile duct cystadenocarcinoma, epithelioid hemangioendothelioma, angiosarcoma, embryonal sarcoma, rhabdomyosarcoma, solitary fibrous tumor, teratoma, York sac tumor, carcinosarcoma, rhabdoid tumor);
kidney (e.g., ALK-rearranged renal cell carcinoma, chromophobe renal cell carcinoma, clear cell renal cell carcinoma, clear cell sarcoma, metanephric adenoma, metanephric adenofibroma, mucinous tubular and spindle cell carcinoma, nephroma, nephroblastoma (Wilms tumor), papillary adenoma, papillary renal cell carcinoma, renal oncocytoma, renal cell carcinoma, succinate dehydrogenase-deficient renal cell carcinoma, collecting duct carcinoma);
breast (e.g., invasive ductal carcinoma, including without limitation, acinic cell carcinoma, adenoid cystic carcinoma, apocrine carcinoma, cribriform carcinoma, glycogen-rich/clear cell, inflammatory carcinoma, lipid-rich carcinoma, medullary carcinoma, metaplastic carcinoma, micropapillary carcinoma, mucinous carcinoma, neuroendocrine carcinoma, oncocytic carcinoma, papillary carcinoma, sebaceous carcinoma, secretory breast carcinoma, tubular carcinoma; lobular carcinoma, including without limitation, pleomorphic carcinoma, signet ring cell carcinoma);
peritoneum (e.g., mesothelioma; primary peritoneal cancer);
female sex organ tissues, including ovary (e.g., choriocarcinoma, epithelial tumors, germ cell tumors, sex cord-stromal tumors), Fallopian tubes (e.g., serous adenocarcinoma, mucinous adenocarcinoma, endometrioid adenocarcinoma, clear cell adenocarcinoma, transitional cell carcinoma, squamous cell carcinoma, undifferentiated carcinoma, mullerian tumors, adenosarcoma, leiomyosarcoma, teratoma, germ cell tumors, choriocarcinoma, trophoblastic tumors), uterus (e.g., carcinoma of the cervix, endometrial polyps, endometrial hyperplasia, intraepithelial carcinoma (EIC), endometrial carcinoma (e.g., endometrioid carcinoma, serous carcinoma, clear cell carcinoma, mucinous carcinoma, squamous cell carcinoma, transitional carcinoma, small cell carcinoma, undifferentiated carcinoma, mesenchymal neoplasia), leiomyoma (e.g., endometrial stromal nodule, leiomyosarcoma, endometrial stromal sarcoma (ESS), mesenchymal tumors), mixed epithelial and mesenchymal tumors (e.g., adenofibroma, carcinofibroma, adenosarcoma, carcinosarcoma (malignant mixed mesodermal sarcoma—MMMT)), endometrial stromal tumors, endometrial malignant mullerian mixed tumours, gestational trophoblastic tumors (partial hydatiform mole, complete hydatiform mole, invasive hydatiform mole, placental site tumour)), vulva, vagina;
male sex organ tissues, including prostate, testis (e.g., germ cell tumors, spermatocytic seminoma), penis;
bladder (e.g., squamous cell carcinoma, urothelial carcinoma, bladder urothelial carcinoma);
brain, (e.g., gliomas (e.g., astrocytomas, including non-infiltrating, low-grade, anaplastic, glioblastomas; oligodendrogliomas, ependymomas), meningiomas, gangliogliomas, schwannomas (neurilemmomas), craniopharyngiomas, chordomas, Non-Hodgkin lymphomas (NHLs), indolent non-Hodgkin's lymphoma (iNHL), refractory iNHL, pituitary tumors;
eye (e.g., retinoma, retinoblastoma, ocular melanoma, posterior uveal melanoma, iris hamartoma);
head and neck (e.g., nasopharyngeal carcinoma, Endolymphatic Sac Tumor (ELST), epidermoid carcinoma, laryngeal cancers including squamous cell carcinoma (SCC) (e.g., glottic carcinoma, supraglottic carcinoma, subglottic carcinoma, transglottic carcinoma), carcinoma in situ, verrucous, spindle cell and basaloid SCC, undifferentiated carcinoma, laryngeal adenocarcinoma, adenoid cystic carcinoma, neuroendocrine carcinomas, laryngeal sarcoma), head and neck paragangliomas (e.g., carotid body, jugulotympanic, vagal);
thymus (e.g., thymoma);
heart (e.g., cardiac myxoma);
lung (e.g., small cell carcinoma (SCLC), non-small cell lung carcinoma (NSCLC), including squamous cell carcinoma (SCC), adenocarcinoma and large cell carcinoma, carcinoids (typical or atypical), carcinosarcomas, pulmonary blastomas, giant cell carcinomas, spindle cell carcinomas, pleuropulmonary blastoma);
lymph (e.g., lymphomas, including Hodgkin's lymphoma, non-Hodgkin's lymphoma (NHL), indolent non-Hodgkin's lymphoma (iNHL), refractory iNHL, Epstein-Barr virus (EBV)-associated lymphoproliferative diseases, including B cell lymphomas and T cell lymphomas (e.g., Burkitt lymphoma; large B cell lymphoma, diffuse large B-cell lymphoma (DLBCL), mantle cell lymphoma, indolent B-cell lymphoma, low grade B cell lymphoma, fibrin-associated diffuse large cell lymphoma; primary effusion lymphoma; plasmablastic lymphoma; extranodal NK/T cell lymphoma, nasal type; peripheral T cell lymphoma, cutaneous T cell lymphoma, angioimmunoblastic T cell lymphoma; follicular T cell lymphoma; systemic T cell lymphoma), lymphangioleiomyomatosis);
central nervous system (CNS) (e.g., gliomas including astrocytic tumors (e.g., pilocytic astrocytoma, pilomyxoid astrocytoma, subependymal giant cell astrocytoma, pleomorphic xanthoastrocytoma, diffuse astrocytoma, fibrillary astrocytoma, gemistocytic astrocytoma, protoplasmic astrocytoma, anaplastic astrocytoma, glioblastoma (e.g., giant cell glioblastoma, gliosarcoma, glioblastoma multiforme) and gliomatosis cerebri), oligodendroglial tumors (e.g., oligodendroglioma, anaplastic oligodendroglioma), oligoastrocytic tumors (e.g., oligoastrocytoma, anaplastic oligoastrocytoma), ependymal tumors (e.g., subependymom, myxopapillary ependymoma, ependymomas (e.g., cellular, papillary, clear cell, tanycytic), anaplastic ependymoma), optic nerve glioma, and non-gliomas (e.g., choroid plexus tumors, neuronal and mixed neuronal-glial tumors, pineal region tumors, embryonal tumors, medulloblastoma, meningeal tumors, primary CNS lymphomas, germ cell tumors, Pituitary adenomas, cranial and paraspinal nerve tumors, stellar region tumors); neurofibroma, meningioma, peripheral nerve sheath tumors, peripheral neuroblastic tumours (including without limitation neuroblastoma, ganglioneuroblastoma, ganglioneuroma), trisomy 19 ependymoma);
neuroendocrine tissues (e.g., paraganglionic system including adrenal medulla (pheochromocytomas) and extra-adrenal paraganglia ((extra-adrenal) paragangliomas); skin (e.g., clear cell hidradenoma, cutaneous benign fibrous histiocytomas, cylindroma, hidradenoma, melanoma (including cutaneous melanoma, mucosal melanoma), basal cell carcinoma, pilomatricoma, Spitz tumors); and soft tissues (e.g., aggressive angiomyxoma, alveolar rhabdomyosarcoma, alveolar soft part sarcoma, angiofibroma, angiomatoid fibrous histiocytoma, synovial sarcoma, biphasic synovial sarcoma, clear cell sarcoma, dermatofibrosarcoma protuberans, desmoid-type fibromatosis, small round cell tumor, desmoplastic small round cell tumor, elastofibroma, embryonal rhabdomyosarcoma, Ewing's tumors/primitive neurectodermal tumors (PNET), extraskeletal myxoid chondrosarcoma, extraskeletal osteosarcoma, paraspinal sarcoma, inflammatory myofibroblastic tumor, lipoblastoma, lipoma, chondroid lipoma, liposarcoma/malignant lipomatous tumors, liposarcoma, myxoid liposarcoma, fibromyxoid sarcoma, lymphangioleiomyoma, malignant myoepithelioma, malignant melanoma of soft parts, myoepithelial carcinoma, myoepithelioma, myxoinflammatory fibroblastic sarcoma, undifferentiated sarcoma, pericytoma, rhabdomyosarcoma, non-rhabdomyosarcoma soft tissue sarcoma (NRSTS), soft tissue leiomyosarcoma, undifferentiated sarcoma, well-differentiated liposarcoma.

In some embodiments, the subject has a hematological cancer, e.g., a leukemia (e.g., Acute Myelogenous Leukemia (AML), Acute Lymphoblastic Leukemia (ALL), B-cell ALL, Myelodysplastic Syndrome (MDS), myeloproliferative disease (MPD), Chronic Myelogenous Leukemia (CML), Chronic Lymphocytic Leukemia (CLL), undifferentiated leukemia), a lymphoma (e.g., small lymphocytic lymphoma (SLL), mantle cell lymphoma (MCL), follicular lymphoma (FL), T-cell lymphoma, B-cell lymphoma, diffuse large B-cell lymphoma (DLBCL), marginal zone lymphoma (MZL), Waldestrom's macroglobulinemia (WM)) and/or a myeloma (e.g., multiple myeloma (MM)).

In some embodiments, the subject has a tumor that is infiltrated with conventional dendritic cells (cDCs). In some embodiments, the tumor infiltrating dendritic cells express C-C motif chemokine receptor 5 (CCR5, CD195) and/or X-C motif chemokine receptor 1 (XCR1) on their cell surface and/or produce CXCL9/10. Expression of XCR1 and CCR5 by cDC1 enables local recruitment by cytotoxic lymphocytes producing the ligands for these chemokine receptors, XCL1 and CCL4/5. cDC1 ability to produce CXCL9/10 promotes local recruitment of effector and memory CTLs expressing CXCR3. Cancel, et al., Front Immunol. (2019) 10:9. In some embodiments, the tumor infiltrating dendritic cells express one or more cell surface proteins selected from the group consisting of XCR1, interferon regulatory factor 8 (IRF8), cell adhesion molecule 1 (CADM1), C-type lectin domain containing 9A (CLEC9A, CD370), and thrombomodulin (THBD), which identify a cDC1 subtype. In some embodiments, the tumor infiltrating dendritic cells express one or more proteins selected from the group consisting of XCR1, IRF8, CADM1, CLEC9A, THBD, copine 3 (CPNE3), carboxypeptidase vitellogenic like (CPVL), N-acylethanolamine acid amidase (NAAA), cystatin C (CST3), WDFY family member 4 (WDFY4) and galectin 2 (LGALS2), which identify a cDC₁ subtype. cDC₁ cells are efficient antigen cross-presenters to CD8+ T cells. In some embodiments, the tumor infiltrating dendritic cells express one or more cell surface proteins selected from the group consisting of CD1A, CD1C, CD1E, signal regulatory protein alpha (SIRPA; CD172A), CD207 and Fc fragment of IgE receptor Ia (FCER1A), which identify a cDC2 subtype. In some embodiments, the tumor infiltrating dendritic cells express one or more cell surface proteins selected from the group consisting of CD1A, CD1C, CD1E, SIRPA, FCER1A, CD207, HLA-DQA2, HLA-DQB2, Fc fragment of IgG binding protein (FCGBP), S100 calcium binding protein B (S100B), NDRG family member 2 (NDRG2), interleukin 22 receptor subunit alpha 2 (IL22RA2), and chondroadherin (CHAD), which identify a cDC2 subtype. cDC2 cells preferentially interact with CD4+ T cells. In some embodiments, the tumor infiltrating dendritic cells expresses one or more proteins selected from the group consisting of basic leucine zipper ATF-like transcription factor 3 (BATF3) and interferon regulatory factor 8 (IRF8), identifying an “activated” DC phenotype or hDC3 subtype. In some embodiments, the tumor infiltrating dendritic cells expresses one or more proteins selected from the group consisting of BATF3, IRF8, C-C motif chemokine ligand 22 (CCL22), lymphocyte antigen 75 (LY75), C-C motif chemokine receptor 7 (CCR7), protein O-glucosyltransferase 1 (POGLUT1), lysine demethylase 2B (KDM2B), INSM transcriptional repressor 1 (INSM1), and UV radiation resistance associated (UVRAG), identifying an “activated” DC phenotype or hDC3 subtype. Expression signatures of various dendritic cell subtypes are described in Zilionis et al., Immunity (2019) 50, 1317-1334. In some embodiments, the tumor infiltrating dendritic cells express one or more cell surface proteins selected from the group consisting of XCR1, BATF3, IRF8, CLEC9A and THBD.

Administration of the FLT3L-Fc proteins described herein can promote or increase expansion and/or infiltration of myeloid cells (e.g., T-cells, NK cells and dendritic cells) into a tumor. Further, administration of the FLT3L-Fc proteins described herein can improve, increase, enhance and/or promote the antitumor effects or efficacy of an immune checkpoint inhibitor. In some embodiments, the subject has a cancer that detectably expresses or overexpresses one or more cell surface immune checkpoint receptors. In certain embodiments, greater than about 50% of the cells within the tumor (e.g., tumor cells, T cells and/or NK cells within the tumor) detectably express one or more cell surface immune checkpoint proteins (e.g., the subject has a so-called “hot” cancer or tumor). In some embodiments, greater than about 1% and less than about 50% of the cells within the tumor (e.g., tumor cells, T cells and/or NK cells within the tumor) detectably express one or more cell surface immune checkpoint proteins (e.g., the subject has a so called “warm” cancer or tumor). In some embodiments, the one or more cell surface immune checkpoint receptors are selected from the group consisting of: CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAET1E; ULBP4); retinoic acid early transcript 1G (RAET1G; ULBP5); retinoic acid early transcript 1L (RAET1L; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); killer cell lectin like receptor G1 (KLRG1; CLEC15A, MAFA, 2F1); sialic acid binding Ig like lectin 7 (SIGLEC7); and sialic acid binding Ig like lectin 9 (SIGLEC9).

The FLT3L-Fc variants described herein can be used to promote or accelerate the recovery from or reverse the effects of lymphopenia or neutropenia. Accordingly, in some embodiments, the subject has neutropenia or lymphopenia, e.g., as a result of having received or undergone a lymphodepleting chemotherapy regimen, e.g., an alkylating agent such as chlorambucil or cyclophosphamide, or a nucleoside analog, including pyrimidine nucleosides such as cytarabine and purine nucleosides such as cladribine, pentostatin and fludarabine. See, e.g., Lowe, et al., Gene Therapy (2018) 25:176-191. In certain embodiments, the methods comprise (a) subjecting a patient to a lymphodepleting chemotherapy regimen; (b) administering a FLT3L-Fc fusion protein, homodimer, heterodimer, conjugate, polynucleotide, vector, lipoplex, such as an LNP, and/or pharmaceutical composition, as described herein; and (c) administering to the patient a cellular therapy, as described herein. Illustrative lymphodepleting chemotherapy regimens, along with correlative beneficial biomarkers, are described in WO 2016/191756 and WO 2019/079564, incorporated herein by reference in their entireties for all purposes. In certain embodiments, the lymphodepleting chemotherapy regimen comprises administering to the patient doses of cyclophosphamide (between 200 mg/m²/day and 2000 mg/m²/day) and doses of fludarabine (between 20 mg/m²/day and 900 mg/m²/day). One such dose regimen involves treating a patient comprising administering daily to the patient about 500 mg/m²/day of cyclophosphamide and about 60 mg/m²/day of fludarabine for three days, e.g., prior to administration of a therapeutically effective amount of a cellular therapy (e.g., an effector cell having a chimeric antigen receptor) to the patient. In another example, in some embodiments, a lymphodepleting chemotherapy regimen of cyclophosphamide 500 mg/m² IV and fludarabine 30 mg/m² IV on the fifth, fourth, and third day e.g., prior to administration of a therapeutically effective amount of a cellular therapy (e.g., an effector cell having a chimeric antigen receptor) to the patient. In some embodiments, the subject is naïve to or has not received chemotherapy. In some embodiments, the subject has bone marrow cells (e.g., is not depleted of bone marrow cells).

In some embodiments, the subject does not have a mutation in the gene encoding the FLT3 receptor that causes or results in or is associated with cancer, e.g., FLT3 mutations associated with constitutive signaling of the FLT3 receptor, e.g., FLT3 mutations associated with Acute Myeloid Leukemia (AML). For example, in certain embodiments, the subject does not have internal tandem duplication (ITD) of the FMS-related tyrosine kinase 3 (FLT3) gene, which occurs in exons 14 and 15, and is one of the most prevalent somatic mutations in adult acute myeloid leukemia (AML). In some embodiments, the subject does not have a mutation in the FLT3 gene in exon 20 that affects codon 835, encoding the tyrosine kinase domain (TKD) mutation, which occurs relatively frequently in adult AML. In some embodiments, the subject does not have point mutations affecting amino acid positions D835 (e.g., resulting in D835Y, D835V, and D835H amino acid substitutions) and/or 1836 in the TKD. See, e.g., Azari-Yam, et al., Clin Lab. (2016) 62(10):2011-2017; Han, et al., Zhongguo Shi Yan Xue Ye Xue Za Zhi. (2009) 17(5):1135-9; Shoji, et al., Rinsho Byori. (2017) 65(1):44-5; and Liang, et al., Leukemia. (2003) 17(5):883-6.

8. Combination Therapies

In various embodiments, the FLT3L-Fc fusion proteins, homodimers, heterodimers, polynucleotides, vectors, lipoplex, such as an LNPs, and/or pharmaceutical compositions, as described herein, are combined with one or more additional therapeutic agents, e.g., an inhibitory immune checkpoint blocker or inhibitor, a stimulatory immune checkpoint stimulator, agonist or activator, a chemotherapeutic agent, an anticancer agent, an antiviral agent, a radiotherapeutic agent, an anti-neoplastic agent, an anti-proliferation agent, an anti-angiogenic agent, an anti-inflammatory agent, an immunotherapeutic agent, a therapeutic antigen-binding molecule (mono- and multi-specific antibodies and fragments thereof in any format (e.g., including without limitation DARTs®, Duobodies®, BiTEs®, BiKEs, TriKEs, XmAbs®, TandAbs®, scFvs, Fabs, Fab derivatives), bi-specific antibodies, non-immunoglobulin antibody mimetics (e.g., including without limitation adnectins, affibody molecules, affilins, affimers, affitins, alphabodies, anticalins, peptide aptamers, armadillo repeat proteins (ARMs), atrimers, avimers, designed ankyrin repeat proteins (DARPins®), fynomers, knottins, Kunitz domain peptides, monobodies, and nanoCLAMPs), antibody-drug conjugates (ADC)), an oncolytic virus, a gene modifier or editor, a cell comprising a chimeric antigen receptor (CAR), e.g., including a T-cell immunotherapeutic agent, an NK-cell immunotherapeutic agent, or a macrophage immunotherapeutic agent, a cell comprising an engineered T-cell receptor (TCR-T), or any combination thereof.

A. Illustrative Targets

In some embodiments, the one or more additional therapeutic agents include, without limitation, an inhibitor, agonist, antagonist, ligand, modulator, stimulator, blocker, activator or suppressor of a target (e.g., polypeptide or polynucleotide) including without limitation: Abelson murine leukemia viral oncogene homolog 1 gene (ABL, such as ABL1), Acetyl-CoA carboxylase (such as ACC1/2), activated CDC kinase (ACK, such as ACK1), Adenosine deaminase, adenosine receptor (such as A2B, A2a, A3), Adenylate cyclase, ADP ribosyl cyclase-1, adrenocorticotropic hormone receptor (ACTH), Aerolysin, AKT1 gene, Alk-5 protein kinase, Alkaline phosphatase, Alpha 1 adrenoceptor, Alpha 2 adrenoceptor, Alpha-ketoglutarate dehydrogenase (KGDH), Aminopeptidase N, AMP activated protein kinase, anaplastic lymphoma kinase (ALK, such as ALK1), Androgen receptor, Angiopoietin (such as ligand-1, ligand-2), Angiotensinogen (AGT) gene, murine thymoma viral oncogene homolog 1 (AKT) protein kinase (such as AKT1, AKT2, AKT3), apolipoprotein A-I (APOA1) gene, Apoptosis inducing factor, apoptosis protein (such as 1, 2), apoptosis signal-regulating kinase (ASK, such as ASK1), Arginase (I), Arginine deiminase, Aromatase, Asteroid homolog 1 (ASTE1) gene, ataxia telangiectasia and Rad 3 related (ATR) serine/threonine protein kinase, Aurora protein kinase (such as 1, 2), Axl tyrosine kinase receptor, 4-1BB ligand (CD137L), Baculoviral IAP repeat containing 5 (BIRC5) gene, Basigin, B-cell lymphoma 2 (BCL2) gene, Bcl2 binding component 3, Bcl2 protein, BCL2L11 gene, BCR (breakpoint cluster region) protein and gene, Beta adrenoceptor, Beta-catenin, B-lymphocyte antigen CD19, B-lymphocyte antigen CD20, B-lymphocyte cell adhesion molecule, B-lymphocyte stimulator ligand, Bone morphogenetic protein-10 ligand, Bone morphogenetic protein-9 ligand modulator, Brachyury protein, Bradykinin receptor, B-Raf proto-oncogene (BRAF), Brc-Abl tyrosine kinase, Bromodomain and external domain (BET) bromodomain containing protein (such as BRD2, BRD3, BRD4), Bruton's tyrosine kinase (BTK), Calmodulin, calmodulin-dependent protein kinase (CaMK, such as CAMKII), Cancer testis antigen 2, Cancer testis antigen NY-ESO-1, cancer/testis antigen 1B (CTAG1) gene, Cannabinoid receptor (such as CB1, CB2), Carbonic anhydrase, casein kinase (CK, such as CKI, CKII), Caspase (such as caspase-3, caspase-7, Caspase-9), caspase 8 apoptosis-related cysteine peptidase CASP8-FADD-like regulator, Caspase recruitment domain protein-15, Cathepsin G, CCR5 gene, CDK-activating kinase (CAK), Checkpoint kinase (such as CHK1, CHK2), chemokine (C-C motif) receptor (such as CCR2, CCR4, CCR5, CCR8), chemokine (C-X-C motif) receptor (such as CXCR1, CXCR2, CXCR3 and CXCR4), Chemokine CC21 ligand, Cholecystokinin CCK2 receptor, Chorionic gonadotropin, c-Kit (tyrosine-protein kinase Kit or CD117), CISH (Cytokine-inducible SH2-containing protein), Claudin (such as 6, 18), cluster of differentiation (CD) such as CD4, CD27, CD29, CD30, CD33, CD37, CD40, CD40 ligand receptor, CD40 ligand, CD40LG gene, CD44, CD45, CD47, CD49b, CD51, CD52, CD55, CD58, CD66e (CEACAM6), CD70 gene, CD74, CD79, CD79b, CD79B gene, CD80, CD95, CD99, CD117, CD122, CDw123, CD134, CDw137, CD158a, CD158b1, CD158b2, CD223, CD276 antigen; clusterin (CLU) gene, Clusterin, c-Met (hepatocyte growth factor receptor (HGFR)), Complement C3, Connective tissue growth factor, COPS signalosome subunit 5, CSF-1 (colony-stimulating factor 1 receptor), CSF2 gene, CTLA-4 (cytotoxic T-lymphocyte protein 4) receptor, C-type lectin domain protein 9A (CLEC9A), Cyclin D1, Cyclin G1, cyclin-dependent kinases (CDK, such as CDK1, CDK1B, CDK2-9), cyclooxygenase (such as COX1, COX2), CYP2B1 gene, Cysteine palmitoyltransferase porcupine, Cytochrome P450 11B2, Cytochrome P450 17, cytochrome P450 17A1, Cytochrome P450 2D6, cytochrome P450 3A4, Cytochrome P450 reductase, cytokine signalling-1, cytokine signalling-3, Cytoplasmic isocitrate dehydrogenase, Cytosine deaminase, cytosine DNA methyltransferase, cytotoxic T-lymphocyte protein-4, DDR2 gene, Death receptor 5 (DR5, TRAILR2), Death receptor 4 (DR4, TRAILR1), Delta-like protein ligand (such as 3, 4), Deoxyribonuclease, Dickkopf-1 ligand, dihydrofolate reductase (DHFR), Dihydropyrimidine dehydrogenase, Dipeptidyl peptidase IV, discoidin domain receptor (DDR, such as DDR1), DNA binding protein (such as HU-beta), DNA dependent protein kinase, DNA gyrase, DNA methyltransferase, DNA polymerase (such as alpha), DNA primase, dUTP pyrophosphatase, L-dopachrome tautomerase, echinoderm microtubule like protein 4, EGFR tyrosine kinase receptor, Elastase, Elongation factor 1 alpha 2, Elongation factor 2, Endoglin, Endonuclease, Endoplasmin, Endosialin, Endostatin, endothelin (such as ET-A, ET-B), Enhancer of zeste homolog 2 (EZH2), Ephrin (EPH) tyrosine kinase (such as Epha3, Ephb4), Ephrin B2 ligand, epidermal growth factor, epidermal growth factor receptors (EGFR), epidermal growth factor receptor (EGFR) gene, Epigen, Epithelial cell adhesion molecule (EpCAM), Erb-b2 (v-erb-b2 avian erythroblastic leukemia viral oncogene homolog 2) tyrosine kinase receptor, Erb-b3 tyrosine kinase receptor, Erb-b4 tyrosine kinase receptor, E-selectin, Estradiol 17 beta dehydrogenase, Estrogen receptor (such as alpha, beta), Estrogen related receptor, Eukaryotic translation initiation factor 5A (EIF5A) gene, Exportin 1, Extracellular signal related kinase (such as 1, 2), Extracellular signal-regulated kinases (ERK), Factor (such as Xa, VIIa), farnesoid x receptor (FXR), Fas ligand, Fatty acid synthase (FASN), Ferritin, FGF-2 ligand, FGF-5 ligand, fibroblast growth factor (FGF, such as FGF1, FGF2, FGF4), Fibronectin, focal adhesion kinase (FAK, such as FAK2), folate hydrolase prostate-specific membrane antigen 1 (FOLH1), Folate receptor (such as alpha), Folate, Folate transporter 1, FYN tyrosine kinase, paired basic amino acid cleaving enzyme (FURIN), Beta-glucuronidase, Galactosyltransferase, Galectin-3, Ganglioside GD2, Glucocorticoid, glucocorticoid-induced TNFR-related protein GITR receptor, Glutamate carboxypeptidase II, glutaminase, Glutathione S-transferase P, glycogen synthase kinase (GSK, such as 3-beta), Glypican 3 (GPC3), gonadotropin-releasing hormone (GNRH), Granulocyte macrophage colony stimulating factor (GM-CSF) receptor, Granulocyte-colony stimulating factor (GCSF) ligand, growth factor receptor-bound protein 2 (GRB2), Grp78 (78 kDa glucose-regulated protein) calcium binding protein, molecular chaperone groEL2 gene, Heme oxygenase 1 (HO1), Heme oxygenase 2 (HO2), Heat shock protein (such as 27, 70, 90 alpha, beta), Heat shock protein gene, Heat stable enterotoxin receptor, Hedgehog protein, Heparanase, Hepatocyte growth factor, HERV-H LTR associating protein 2, Hexose kinase, Histamine H2 receptor, Histone methyltransferase (DOT1L), histone deacetylase (HDAC, such as 1, 2, 3, 6, 10, 11), Histone H1, Histone H3, HLA class I antigen (A-2 alpha), HLA class II antigen, HLA class I antigen alpha G (HLA-G), Non-classical HLA, Homeobox protein NANOG, HSPB1 gene, Human leukocyte antigen (HLA), Human papillomavirus (such as E6, E7) protein, Hyaluronic acid, Hyaluronidase, Hypoxia inducible factor-1 alpha (HIF1α), Imprinted Maternally Expressed Transcript (H19) gene, mitogen-activated protein kinase kinase kinase kinase 1 (MAP4K1), tyrosine-protein kinase HCK, I-Kappa-B kinase (IKK, such as IKKbe), IL-1 alpha, IL-1 beta, IL-12, IL-12 gene, IL-15, IL-17, IL-2 gene, IL-2 receptor alpha subunit, IL-2, IL-3 receptor, IL-4, IL-6, IL-7, IL-8, immunoglobulin (such as G, G1, G2, K, M), Immunoglobulin Fc receptor, Immunoglobulin gamma Fc receptor (such as I, III, IIIA), indoleamine 2,3-dioxygenase (IDO, such as IDO1 and IDO2), indoleamine pyrrole 2,3-dioxygenase 1 inhibitor, insulin receptor, Insulin-like growth factor (such as 1, 2), Integrin alpha-4/beta-1, integrin alpha-4/beta-7, Integrin alpha-5/beta-1, Integrin alpha-V/beta-3, Integrin alpha-V/beta-5, Integrin alpha-V/beta-6, Intercellular adhesion molecule 1 (ICAM-1), interferon (such as alpha, alpha 2, beta, gamma), Interferon inducible protein absent in melanoma 2 (AIM2), interferon type I receptor, Interleukin 1 ligand, Interleukin 13 receptor alpha 2, interleukin 2 ligand, interleukin-1 receptor-associated kinase 4 (IRAK4), Interleukin-2, Interleukin-29 ligand, isocitrate dehydrogenase (such as IDH1, IDH2), Janus kinase (JAK, such as JAK1, JAK2), Jun N terminal kinase, kallikrein-related peptidase 3 (KLK3) gene, Killer cell Ig like receptor, Kinase insert domain receptor (KDR), Kinesin-like protein KIF11, Kirsten rat sarcoma viral oncogene homolog (KRAS) gene, Kisspeptin (KiSS-1) receptor, KIT gene, v-kit Hardy-Zuckerman 4 feline sarcoma viral oncogene homolog (KIT) tyrosine kinase, lactoferrin, Lanosterol-14 demethylase, LDL receptor related protein-1, Leukocyte immunoglobulin-like receptor subfamily B member 1 (ILT2), Leukocyte immunoglobulin-like receptor subfamily B member 2 (ILT4), Leukotriene A4 hydrolase, Listeriolysin, L-Selectin, Luteinizing hormone receptor, Lyase, lymphocyte activation gene 3 protein (LAG-3), Lymphocyte antigen 75, Lymphocyte function antigen-3 receptor, lymphocyte-specific protein tyrosine kinase (LCK), Lymphotactin, Lyn (Lck/Yes novel) tyrosine kinase, lysine demethylases (such as KDM1, KDM2, KDM4, KDM5, KDM6, A/B/C/D), Lysophosphatidate-1 receptor, lysosomal-associated membrane protein family (LAMP) gene, Lysyl oxidase homolog 2, lysyl oxidase protein (LOX), lysyl oxidase-like protein (LOXL, such as LOXL2), 5-Lipoxygenase (5-LOX), Hematopoietic Progenitor Kinase 1 (HPK1), Hepatocyte growth factor receptor (MET) gene, macrophage colony-stimulating factor (MCSF) ligand, Macrophage migration inhibitory fact, MAGEC1 gene, MAGEC2 gene, Major vault protein, MAPK-activated protein kinase (such as MK2), Mas-related G-protein coupled receptor, matrix metalloprotease (MMP, such as MMP2, MMP9), Mcl-1 differentiation protein, Mdm2 p53-binding protein, Mdm4 protein, Melan-A (MART-1) melanoma antigen, Melanocyte protein Pmel 17, melanocyte stimulating hormone ligand, melanoma antigen family A3 (MAGEA3) gene, Melanoma associated antigen (such as 1, 2, 3, 6), Membrane copper amine oxidase, Mesothelin, MET tyrosine kinase, Metabotropic glutamate receptor 1, Metalloreductase STEAP1 (six transmembrane epithelial antigen of the prostate 1), Metastin, methionine aminopeptidase-2, Methyltransferase, Mitochondrial 3 ketoacyl CoA thiolase, mitogen-activate protein kinase (MAPK), mitogen-activated protein kinase (MEK, such as MEK1, MEK2), mTOR (mechanistic target of rapamycin (serine/threonine kinase), mTOR complex (such as 1,2), mucin (such as 1, 5A, 16), mut T homolog (MTH, such as MTH1), Myc proto-oncogene protein, myeloid cell leukemia 1 (MCL1) gene, myristoylated alanine-rich protein kinase C substrate (MARCKS) protein, NAD ADP ribosyltransferase, natriuretic peptide receptor C, Neural cell adhesion molecule 1, Neurokinin 1 (NK1) receptor, Neurokinin receptor, Neuropilin 2, NF kappa B activating protein, NIMA-related kinase 9 (NEK9), Nitric oxide synthase, NK cell receptor, NK3 receptor, NKG2 A B activating NK receptor, NLRP3 (NACHT LRR PYD domain protein 3) modulators, Noradrenaline transporter, Notch (such as Notch-2 receptor, Notch-3 receptor, Notch-4 receptor), Nuclear erythroid 2-related factor 2, Nuclear Factor (NF) kappa B, Nucleolin, Nucleophosmin, nucleophosmin-anaplastic lymphoma kinase (NPM-ALK), 2 oxoglutarate dehydrogenase, 2,5-oligoadenylate synthetase, O-methylguanine DNA methyltransferase, Opioid receptor (such as delta), Ornithine decarboxylase, Orotate phosphoribosyltransferase, orphan nuclear hormone receptor NR4A1, Osteocalcin, Osteoclast differentiation factor, Osteopontin, OX-40 (tumor necrosis factor receptor superfamily member 4 TNFRSF4, or CD134) receptor, P3 protein, p38 kinase, p38 MAP kinase, p53 tumor suppressor protein, Parathyroid hormone ligand, peroxisome proliferator-activated receptors (PPAR, such as alpha, delta, gamma), P-Glycoprotein (such as 1), phosphatase and tensin homolog (PTEN), phosphatidylinositol 3-kinase (PI3K), phosphoinositide-3 kinase (PI3K such as alpha, delta, gamma), phosphorylase kinase (PK), PKN3 gene, placenta growth factor, platelet-derived growth factor (PDGF, such as alpha, beta), Platelet-derived growth factor (PDGF, such as alpha, beta), Pleiotropic drug resistance transporter, Plexin B1, PLK1 gene, polo-like kinase (PLK), Polo-like kinase 1, Poly (ADP-ribose) polymerase (PARP, such as PARP1, PARP2 and PARP3, PARP7, and mono-PARPs), Preferentially expressed antigen in melanoma (PRAME) gene, Prenyl-binding protein (PrPB), Probable transcription factor PML, Progesterone receptor, Programmed cell death 1 (PD-1), Programmed cell death ligand 1 inhibitor (PD-L1), Prosaposin (PSAP) gene, Prostanoid receptor (EP4), Prostaglandin E2 synthase, prostate specific antigen, Prostatic acid phosphatase, proteasome, Protein E7, Protein farnesyltransferase, protein kinase (PK, such as A, B, C), protein tyrosine kinase, Protein tyrosine phosphatase beta, Proto-oncogene serine/threonine-protein kinase (PIM, such as PIM-1, PIM-2, PIM-3), P-Selectin, Purine nucleoside phosphorylase, purinergic receptor P2X ligand gated ion channel 7 (P2X7), Pyruvate dehydrogenase (PDH), Pyruvate dehydrogenase kinase, Pyruvate kinase (PYK), 5-Alpha-reductase, Raf protein kinase (such as 1, B), RAF1 gene, Ras gene, Ras GTPase, RET gene, Ret tyrosine kinase receptor, retinoblastoma associated protein, retinoic acid receptor (such as gamma), Retinoid X receptor, Rheb (Ras homolog enriched in brain) GTPase, Rho (Ras homolog) associated protein kinase 2, ribonuclease, Ribonucleotide reductase (such as M2 subunit), Ribosomal protein S6 kinase, RNA polymerase (such as I, II), Ron (Recepteur d'Origine Nantais) tyrosine kinase, ROS1 (ROS proto-oncogene 1, receptor tyrosine kinase) gene, Rosl tyrosine kinase, Runt-related transcription factor 3, Gamma-secretase, S100 calcium binding protein A9, Sarco endoplasmic calcium ATPase, Second mitochondria-derived activator of caspases (SMAC) protein, Secreted frizzled related protein-2, Secreted phospholipase A2, Semaphorin-4D, Serine protease, serine/threonine kinase (STK), serine/threonine-protein kinase (TBK, such as TBK1), signal transduction and transcription (STAT, such as STAT-1, STAT-3, STAT-5), Signaling lymphocytic activation molecule (SLAM) family member 7, six-transmembrane epithelial antigen of the prostate (STEAP) gene, SL cytokine ligand, smoothened (SMO) receptor, Sodium iodide cotransporter, Sodium phosphate cotransporter 2B, Somatostatin receptor (such as 1, 2, 3, 4, 5), Sonic hedgehog protein, Son of sevenless (SOS), Specific protein 1 (Sp1) transcription factor, Sphingomyelin synthase, Sphingosine kinase (such as 1, 2), Sphingosine-1-phosphate receptor-1, spleen tyrosine kinase (SYK), SRC gene, Src tyrosine kinase, STAT3 gene, Steroid sulfatase, Stimulator of interferon genes (STING) receptor, stimulator of interferon genes protein, Stromal cell-derived factor 1 ligand, SUMO (small ubiquitin-like modifier), Superoxide dismutase, Suppressor of cytokine signaling modulators (SOCS), Survivin protein, Synapsin 3, Syndecan-1, Synuclein alpha, T cell surface glycoprotein CD28, tank-binding kinase (TBK), TATA box-binding protein-associated factor RNA polymerase I subunit B (TAF1B) gene, T-cell CD3 glycoprotein zeta chain, T-cell differentiation antigen CD6, T-cell immunoglobulin and mucin-domain containing-3 (TIM-3), T-cell surface glycoprotein CD8, Tec protein tyrosine kinase, Tek tyrosine kinase receptor, telomerase, Telomerase reverse transcriptase (TERT) gene, Tenascin, Thrombopoietin receptor, Thymidine kinase, Thymidine phosphorylase, Thymidylate synthase, Thymosin (such as alpha 1), Thyroid hormone receptor, Thyroid stimulating hormone receptor, Tissue factor, TNF related apoptosis inducing ligand, TNFR1 associated death domain protein, TNF-related apoptosis-inducing ligand (TRAIL) receptor, TNFSF11 gene, TNFSF9 gene, Toll-like receptor (TLR such as 1-13), topoisomerase (such as I, II, III), Transcription factor, Transferase, transferrin (TF); transforming growth factor beta 1 (TGFB1) and isoforms thereof, TGF beta 2 ligand, Transforming growth factor TGF-β receptor kinase, Transglutaminase, Translocation associated protein, Transmembrane glycoprotein NMB, Trop-2 calcium signal transducer, trophoblast glycoprotein (TPBG) gene, Trophoblast glycoprotein, Tropomyosin receptor kinase (Trk) receptor (such as TrkA, TrkB, TrkC), Tryptophan 5-hydroxylase, Tubulin, Tumor necrosis factor (TNF, such as alpha, beta), Tumor necrosis factor 13C receptor, tumor progression locus 2 (TPL2), Tumor protein 53 (TP53) gene, Tumor suppressor candidate 2 (TUSC2) gene, Tumor specific neoantigens, Tyrosinase, Tyrosine hydroxylase, tyrosine kinase (TK), Tyrosine kinase receptor, Tyrosine kinase with immunoglobulin-like and EGF-like domains (TIE) receptor, Tyrosine protein kinase ABL1 inhibitor, Ubiquitin, Ubiquitin carboxyl hydrolase isozyme L5, Ubiquitin thioesterase-14, Ubiquitin-conjugating enzyme E2I (UBE2I, UBC9), Urease, Urokinase plasminogen activator, Uteroglobin, Vanilloid VR1, Vascular cell adhesion protein 1, vascular endothelial growth factor receptor (VEGFR), V-domain Ig suppressor of T-cell activation (VISTA), VEGF-1 receptor, VEGF-2 receptor, VEGF-3 receptor, VEGF-A, VEGF-B, Vimentin, Vitamin D3 receptor, Proto-oncogene tyrosine-protein kinase, Mer (Mer tyrosine kinase receptor modulators), YAP (Yes-associated protein modulators)es, Wee-1 protein kinase, Wilms' tumor antigen 1, Wilms' tumor protein, WW domain containing transcription regulator protein 1 (TAZ), X-linked inhibitor of apoptosis protein, Zinc finger protein transcription factor or any combination thereof.

In some embodiments, the one or more additional therapeutic agents include without limitation bispecific antibodies and “antibody-like” therapeutic proteins (such as DARTs®, DUOBODIES®, BITES®, XmAbs®, TandAbs®, Fab derivatives, or TCR-like antibodies), cyclophilin inhibitor, stimulator of retinoic acid-inducible gene 1, stimulator of RIG-I like receptor, PD-1 inhibitor, PD-L1 inhibitor, arginase inhibitor, PI3K inhibitor, IDO inhibitor, stimulator of NOD2, HBV viral entry inhibitors, NTCP inhibitor, HBx inhibitor, cccDNA inhibitor, HBV antibody targeting the surface antigens of the hepatitis B virus, siRNA, miRNA gene therapy agents, sshRNAs, KDM5 inhibitors, or nucleoprotein modulator (HBV core or capsid protein modulator).

B. Illustrative Mechanisms of Action

In various embodiments, the one or more additional therapeutic agents may be categorized by their mechanism of action into, for example, the following groups:
anti-metabolites/anticancer agents, such as pyrimidine analogs floxuridine, capecitabine, cytarabine, CPX-351 (liposomal cytarabine, daunorubicin), and TAS-118;
purine analogs, folate antagonists (such as pralatrexate), cladribine, pentostatin, fludarabine and related inhibitors;
antiproliferative/antimitotic agents including natural products, such as vinca alkaloids (vinblastine, vincristine) and microtubule disruptors such as taxane (paclitaxel, docetaxel), vinblastin, nocodazole, epothilones, vinorelbine (NAVELBINE®), and epipodophyllotoxins (etoposide, teniposide);
DNA damaging agents, such as actinomycin, amsacrine, busulfan, carboplatin, chlorambucil, cisplatin, cyclophosphamide (CYTOXAN®), dactinomycin, daunorubicin, doxorubicin, epirubicin, iphosphamide, melphalan, merchlorethamine, mitomycin C, mitoxantrone, nitrosourea, procarbazine, taxol, Taxotere, teniposide, etoposide, and triethylenethiophosphoramide;
DNA-hypomethylating agents, such as guadecitabine (SGI-110), ASTX727;
antibiotics such as dactinomycin, daunorubicin, doxorubicin, idarubicin, anthracyclines, mitoxantrone, bleomycins, plicamycin (mithramycin);
enzymes such as L-asparaginase which systemically metabolizes L-asparagine and deprives cells which do not have the capacity to synthesize their own asparagine;
DNAi oligonucleotides targeting Bcl-2, such as PNT2258; agents that activate or reactivate latent human immunodeficiency virus (HIV), such as panobinostat and romidepsin;
asparaginase stimulators, such as crisantaspase (Erwinase®) and GRASPA (ERY-001, ERY-ASP), calaspargase pegol;
pan-Trk, ROS1 and ALK inhibitors, such as entrectinib, TPX-0005; anaplastic lymphoma kinase (ALK) inhibitors, such as alectinib, ceritinib;
antiproliferative/antimitotic alkylating agents, such as nitrogen mustard cyclophosphamide and analogs (e.g., melphalan, chlorambucil, hexamethylmelamine, thiotepa), alkyl nitrosoureas (e.g., carmustine) and analogs, streptozocin, and triazenes (e.g., dacarbazine);
antiproliferative/antimitotic antimetabolites, such as folic acid analogs (methotrexate);
platinum coordination complexes (e.g., cisplatin, oxiloplatinim, and carboplatin), procarbazine, hydroxyurea, mitotane, and aminoglutethimide;
hormones, hormone analogs (e.g., estrogen, tamoxifen, goserelin, bicalutamide, and nilutamide), and aromatase inhibitors (e.g., letrozole and anastrozole);
antiplatelet agents; anticoagulants such as heparin, synthetic heparin salts, and other inhibitors of thrombin;
fibrinolytic agents such as tissue plasminogen activator, streptokinase, urokinase, aspirin, dipyridamole, ticlopidine, and clopidogrel;
antimigratory agents; antisecretory agents (e.g., breveldin);
immunosuppressives, such as tacrolimus, sirolimus, azathioprine, and mycophenolate;
growth factor inhibitors, and vascular endothelial growth factor inhibitors;
fibroblast growth factor inhibitors, such as FPA14;
anti-VEGFR antibodies, such as IMC-3C5, GNR-011, tanibirumab, LYN-00101;
anti-VEGF/DDL4 antibodies, such as ABT-165;
anti-cadherin antibodies, such as HKT-288;
anti-CD52 antibodies, such as alemtuzumab;
anti-CD70 antibodies, such as AMG-172;
anti-leucine-rich repeat containing 15 (LRRC15) antibodies, such as ABBV-085, ARGX-110;
angiotensin receptor blockers, nitric oxide donors;
antisense oligonucleotides, such as AEG35156, IONIS-KRAS-2.5Rx, EZN-3042, RX-0201, IONIS-AR-2.5Rx, BP-100 (prexigebersen), IONIS-STAT3-2.5Rx;
DNA interference oligonucleotides, such as PNT2258, AZD-9150;
anti-angiopoietin (ANG)-2 antibodies, such as MEDI3617, and LY3127804;
anti-ANG-1/ANG-2 antibodies, such as AMG-780;
anti-CSF1R antibodies, such as emactuzumab, LY3022855, AMG-820, FPA-008 (cabiralizumab);
anti-CD40 antibodies, such as RG7876, SEA-CD40, APX-005M, ABBV-428;
anti-endoglin antibodies, such as TRC105 (carotuximab);
anti-CD45 antibodies, such as 131I-BC8 (lomab-B); anti-HER3 antibodies, such as LJM716, GSK2849330;
anti-MET/EGFR antibodies, such as LY3164530;
anti-EGFR antibodies, such as ABT-414, AMG-595, necitumumab, ABBV-221, depatuxizumab mafodotin (ABT-414), tomuzotuximab, ABT-806, vectibix, modotuximab, RM-1929;
anti-HER2 antibodies, such as HERCEPTIN® (trastuzumab), margetuximab, MEDI4276, BAT-8001, Pertuzumab (Perjeta), ZW25 (a bispecific HER2-directed antibody targeting the extracellular domains 2 and 4; Cancer Discov. 2019 January; 9(1):8; PMID: 30504239);
HER2 inhibitors, such as neratinib, tucatinib (ONT-380);
EGFR/ErbB2/Ephb4 inhibitors, such as tesevatinib;
anti-ERBB antibodies, such as CDX-3379, HLX-02, seribantumab;
EGFR/ErbB-2 inhibitors, such as varlitinib;
Mutant selective EGFR inhibitors, such as PF-06747775, EGF816 (nazartinib), ASP8273, ACEA-0010, BI-1482694;
anti-HLA-DR antibodies, such as IMMU-114;
anti-IL-3 antibodies, such as JNJ-56022473;
anti-TNF receptor superfamily member 4 (TNFRSF4, OX40; NCBI Gene ID: 7293) antibodies, such as MEDI6469, MEDI6383, MEDI0562 (tavolixizumab), MOXR0916, PF-04518600, RG-7888, GSK-3174998, INCAGN1949, BMS-986178, GBR-8383, ABBV-368; and those described in Intl. Patent Publ. Nos. WO 2016/179517, WO 2017/096179, WO 2017/096182, WO 2017/096281 and WO 2018/089628;
anti-TNF receptor superfamily member 18 (TNFRSF18, GITR; NCBI Gene ID: 8784) antibodies, such as MEDI1873, FPA-154, INCAGN-1876, TRX-518, BMS-986156, MK-1248, GWN-323; and those described, e.g., in Intl. Patent Publ. Nos. WO 2017/096179, WO 2017/096276, WO 2017/096189; and WO 2018/089628;
anti-TNFRSF4 (OX40)/TNFRSF18(GITR) bi-specific antibodies, such as those described in Intl. Patent Publ. Nos. WO 2017/096179 and WO 2018/089628;
anti-EphA3 antibodies, such as KB-004;
anti-CD20 antibodies, such as obinutuzumab, IGN-002;
anti-CD37 antibodies, such as AGS67E, otlertuzumab (TRU-016);
anti-ENPP3 antibodies, such as AGS-16C3F;
anti-FGFR-3 antibodies, such as LY3076226, B-701;
anti-FGFR-2 antibodies, such as GAL-F2;
anti-05 antibodies, such as ALXN-1210;
anti-CD27 antibodies, such as varlilumab (CDX-1127);
anti-TROP-2 antibodies, such as IMMU-132;
anti-NKG2a antibodies, such as monalizumab;
anti-VISTA antibodies, such as HMBD-002;
anti-PVRIG antibodies, such as COM-701;
anti-EpCAM antibodies, such as VB4-845;
antibodies against TNF receptor superfamily member 17 (TNFRSF17, BCMA), such as GSK-2857916;
anti-CEA antibodies, such as RG-7813;
anti-cluster of differentiation 3 (CD3) antibodies, such as MGD015; anti-folate receptor alpha antibodies, such as IMGN853;
epha2 inhibitors, such as MM-310;
anti LAG-3 (Lymphocyte-activation) antibodies, such as relatlimab (ONO-4482), LAG-525, MK-4280, REGN-3767, INCAGN2385;
raf kinase/VEGFR inhibitors, such as RAF-265;
polycomb protein (EED) inhibitors, such as MAK683;
anti-fibroblast activation protein (FAP)/IL-2R antibodies, such as RG7461;
anti-fibroblast activation protein (FAP)/TRAIL-R2 antibodies, such as RG7386;
anti-fucosyl-GM1 antibodies, such as BMS-986012;
p38 MAP kinase inhibitors, such as ralimetinib;
PRMT1 inhibitors, such as MS203;
Sphingosine kinase 2 (SK2) inhibitors, such as opaganib;
Nuclear erythroid 2-related factor 2 stimulators, such as omaveloxolone (RTA-408);
Tropomyosin receptor kinase (TRK) inhibitors, such as LOXO-195, ONO-7579;
anti-ICOS antibodies, such as JTX-2011, GSK3359609;
ICOS agonists, such as ICOS-L.COMP (Gariepy, J. et al. 106th Annu Meet Am Assoc Immunologists (AAI) (May 9-13, San Diego) 2019, Abst 71.5);
anti-TNF receptor superfamily member 10b (TNFRSF10B, DR5, TRAILR2) antibodies, such as DS-8273, CTB-006, INBRX-109, GEN-1029;
anti-Carcinoembryonic-antigen-related-cell-adhesion-molecule-6 (CEACAM6, CD66C) antibodies, such as BAY-1834942, NEO-201 (CEACAM 5/6);
anti-GD2 antibodies, such as APN-301;
anti-interleukin-17 (IL-17) antibodies, such as CJM-112;
anti-carbonic anhydrase 9 (CA9, CAIX) antibodies, such as TX-250;
anti-CD38 antibodies, such as isatuximab, MOR-202;
anti-CD38-attenukine, such as TAK573;
anti-Mucin 1 (MUC1) antibodies, such as gatipotuzumab, Mab-AR-20.5;
Mucin 1 inhibitors, such as GO-203-2C;
MARCK5 protein inhibitors, such as BIO-11006;
Folate antagonists, such as arfolitixorin;
Galectin-3 inhibitors, such as GR-MD-02;
Phosphorylated P68 inhibitors, such as RX-5902;
CD95/TNF modulators, such as ofranergene obadenovec;
PI3K/Akt/mTOR inhibitors, such as ABTL-0812;
pan-PIM kinase inhibitors, such as INCB-053914;
IL-12 gene stimulators, such as EGEN-001, tavokinogene telseplasmid;
Heat shock protein HSP90 inhibitors, such as TAS-116, PEN-866;
VEGF/HGF antagonists, such as MP-0250;
SYK tyrosine kinase/JAK tyrosine kinase inhibitors, such as ASN-002;
JAK3/JAK1/TBK1 kinase inhibitors, such as CS-12912;
IL-24 antagonist, such as AD-IL24;
NLRP3 (NACHT LRR PYD domain protein 3) modulators, such as BMS-986299;
RIG-I agonists, such as RGT-100;
Aerolysin stimulators, such as topsalysin;
P-Glycoprotein 1 inhibitors, such as HM-30181A;
CSF-1 antagonists, such as ARRY-382, BLZ-945;
CCR8 inhibitors, such as I-309, SB-649701, HG-1013, RAP-310;
anti-CCR8 antibodies, such as neutralizing anti-CCR8 antibodies, or anti-CCR8 antibodies having ADCC activity;
anti-Mesothelin antibodies, such as SEL-403;
Thymidine kinase stimulators, such as aglatimagene besadenovec;
Polo-like kinase 1 inhibitors, such as PCM-075;
NEDD8 inhibitors, such as pevonedistat (MLN-4924), TAS-4464;
Pleiotropic pathway modulators, such as avadomide (CC-122);
FoxM1 inhibitors, such as thiostrepton;
UBA1 inhibitors, such as TAK-243;
Src tyrosine kinase inhibitors, such as VAL-201;
VDAC/HK inhibitors, such as VDA-1102;
BRAF/PI3K inhibitors, such as ASN-003;
Elf4a inhibitors, such as rohinitib, eFT226;
TP53 gene stimulators, such as ad-p53;
Retinoic acid receptor alpha (RARα) inhibitors, such as SY-1425;
SIRT3 inhibitors, such as YC8-02;
Stromal cell-derived factor 1 ligand inhibitors, such as olaptesed pegol (NOX-A12);
IL-4 receptor modulators, such as MDNA-55;
Arginase-I stimulators, such as pegzilarginase;
Topoisomerase I inhibitor/hypoxia inducible factor-1 alpha inhibitors, such as PEG-SN38 (firtecan pegol);
Hypoxia inducible factor-1 alpha inhibitors, such as PT-2977, PT-2385;
CD122 agonists, such as NKTR-214;
TLR7/TLR8 agonist, such as NKTR-262;
TLR7 agonists, such as DS-0509, GS-9620 (versatolimod), LHC-165, TMX-101 (imiquimod);
p53 tumor suppressor protein stimulators such as kevetrin;
Mdm4/Mdm2 p53-binding protein inhibitors, such as ALRN-6924;
kinesin spindle protein (KSP) inhibitors, such as filanesib (ARRY-520);
CD80-fc fusion protein inhibitors, such as FPT-155;
Menin and mixed lineage leukemia (MLL) inhibitors such as KO-539;
Liver x receptor agonists, such as RGX-104;
IL-10 agonists, such as AM-0010;
VEGFR/PDGFR inhibitors, such as vorolanib;
IRAK4 inhibitors, such as CA-4948;
anti-TLR-2 antibodies, such as OPN-305;
Calmodulin modulators, such as CBP-501;
Glucocorticoid receptor antagonists, such as relacorilant (CORT-125134);
Second mitochondria-derived activator of caspases (SMAC) protein inhibitors, such as BI-891065;
Lactoferrin modulators, such as LTX-315;
KIT proto-oncogene, receptor tyrosine kinase (KIT) inhibitors, such as PLX-9486;
platelet derived growth factor receptor alpha (PDGFRA)/KIT proto-oncogene, receptor tyrosine kinase (KIT) mutant-specific antagonists/inhibitors such as BLU-285, DCC-2618;
Exportin 1 inhibitors, such as eltanexor;
anti-CD33 antibodies, such as IMGN-779;
anti-KMA antibodies, such as MDX-1097;
anti-TIM-3 antibodies, such as TSR-022, LY-3321367, MBG-453;
anti-CD55 antibodies, such as PAT-SC1;
anti-PSMA antibodies, such as ATL-101;
anti-CD100 antibodies, such as VX-15;
anti-EPHA3 antibodies, such as fibatuzumab;
anti-APRIL antibodies, such as BION-1301;
anti-TIGIT antibodies, such as BMS-986207, tiragolumab (RG-6058), AGEN-1307 (AGEN-1327), AGEN-1777, domvanalimab (AB154), AB-308, etigilimab, vibostolimab;
anti-TIM-3 antibodies, such as INCAGN-2390;
CHST15 gene inhibitors, such as STNM-01;
RAS inhibitors, such as NEO-100;
Somatostatin receptor antagonist, such as OPS-201;
CEBPA gene stimulators, such as MTL-501;
DKK3 gene modulators, such as MTG-201;
Chemokine (CXCR1/CXCR2) inhibitors, such as SX-682;
p70s6k inhibitors, such as MSC2363318A;
methionine aminopeptidase 2 (MetAP2) inhibitors, such as M8891, APL-1202;
arginine N-methyltransferase 5 inhibitors, such as GSK-3326595;
anti-programmed cell death protein 1 (anti-PD-1) antibodies, such as nivolumab (OPDIVO®, BMS-936558, MDX-1106), pembrolizumab (KEYTRUDA®, MK-3477, SCH-900475, lambrolizumab, CAS Reg. No. 1374853-91-4), pidilizumab, PF-06801591, BGB-A317 (tislelizumab), GLS-010 (WBP-3055), AK-103 (HX-008), CS-1003, HLX-10, MGA-012, BI-754091, REGN-2810 (cemiplimab), AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, AK-105, PD1-PIK, BAT-1306, zimberelimab, and anti-programmed death-ligand 1 (anti-PD-L1) antibodies such as BMS-936559, atezolizumab (MPDL3280A), durvalumab (MEDI-4736), avelumab, CK-301 (MSB0010718C), MEDI-0680, CX-072, CBT-502, PDR-001 (spartalizumab), TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, and MDX1105-01;
PD-L1/VISTA antagonists such as CA-170;
PD-1/PD-L1 inhibitors, such as INCB086550, GS-4224, GS-4416;
anti-PD-L1/TGFβ antibodies, such as M-7824;
PD-L1/EGFR inhibitors, such as GNS-1480 (lazertinib);
PD-1/CTLA-4 inhibitors, such as PF-06936308;
anti-CD73/TGFβ inhibitors, such as GS-1423 (AGEN1423; published in WO2019/173692);
anti-CTLA-4 (cytotoxic T-lymphocyte protein-4) antibodies, such as tremelimumab, ipilimumab (BMS-734016), AGEN-1884, BMS-986218, AGEN1181, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BA-3071;
CTLA-4 (cytotoxic T-lymphocyte protein-4) inhibitors, such as BPI-002; TLR-3 agonist/interferon inducers, such as Poly-ICLC (NSC-301463);
anti-transferrin antibodies, such as CX-2029;
anti-IL-8 (Interleukin-8) antibodies, such as HuMax-Inflam;
ATM (ataxia telangiectasia) inhibitors, such as AZD0156;
CHK1 inhibitors, such as GDC-0575, LY2606368 (prexasertib), SRA737, RG7741 (CHK1/2);
CXCR4 antagonists, such as BL-8040, LY2510924, burixafor (TG-0054), X4P-002, X4P-00140, Plerixafor;
EXH2 inhibitors, such as GSK2816126;
KDM1 inhibitors, such as ORY-1001, IMG-7289, INCB-59872, GSK-2879552;
CXCR2 antagonists, such as AZD-5069;
GM-CSF antibodies, such as lenzilumab;
DNA dependent protein kinase inhibitors, such as MSC2490484A (nedisertib), VX-984, AsiDNA (DT-01); protein kinase C (PKC) inhibitors, such as LXS-196, sotrastaurin;
Selective estrogen receptor downregulators (SERD), such as fulvestrant (Faslodex®), RG6046, RG6047, elacestrant (RAD-1901) and AZD9496;
Selective estrogen receptor covalent antagonists (SERCAs), such as H3B-6545;
selective androgen receptor modulator (SARM), such as GTX-024, darolutamide;
transforming growth factor-beta (TGF-beta) kinase antagonists, such as galunisertib; TGF-beta inhibitors described in WO 2019/103203;
anti-transforming growth factor-beta (TGF-beta) antibodies, such as LY3022859, NIS793, XOMA 089, SRK-181;
bispecific antibodies, such as MM-141 (IGF-1/ErbB3), MM-111 (Erb2/Erb3), JNJ-64052781 (CD19/CD3), PRS-343 (CD-137/HER2), AFM26 (BCMA/CD16A), JNJ-61186372 (EGFR/cMET), AMG-211 (CEA/CD3), RG7802 (CEA/CD3), ERY-974 (CD3/GPC3) vancizumab (angiopoietins/VEGF), PF-06671008 (Cadherins/CD3), AFM-13 (CD16/CD30), APV0436 (CD123/CD3), flotetuzumab (CD123/CD3), REGN-1979 (CD20/CD3), MCLA-117 (CD3/CLEC12A), MCLA-128 (HER2/HER3), JNJ-0819, JNJ-7564 (CD3/heme), AMG-757 (DLL3-CD3), MGD-013 (PD-1/LAG-3), FS-118 (LAG-3/PD-L1) MGD-019 (PD-1/CTLA-4), KN-046 (PD-1/CTLA-4), MEDI-5752 (CTLA-4/PD-1), RO-7121661 (PD-1/TIM-3), XmAb-20717 (PD-1/CTLA-4), AK-104 (CTLA-4/PD-1), AMG-330 (CD33/CD3), AMG-420 (BCMA/CD3), BI-836880 (VEFG/ANG2), JNJ-63709178 (CD123/CD3), MGD-007 (CD3/gpA33), MGD-009 (CD3/B7H3), AGEN1223, IMCgp100 (CD3/gp100), AGEN-1423 (GS-1423; CD73/TGF-beta), ATOR-1015 (CTLA-4/OX40), LY-3415244 (TIM3/PDL1), INHIBRX-105 (4-1BB/PDL1), faricimab (VEGF-A/ANG-2), FAP-4-IBBL (4-1BB/FAP), XmAb-13676 (CD3/CD20), TG-1801 (CD19/CD47), XmAb-18087 (SSTR2/CD3), catumaxomab (CD3/EpCAM), SAR-156597 (IL4/IL13), EMB-01 (EGFR/cMET), REGN-4018 (MUC16/CD3), RG-7828 (CD20/CD3), CC-93269 (CD3/BCMA), REGN-5458 (CD3/BCMA), navicixizumab (DLL4/VEGF), GRB-1302 (CD3/Erbb2), vanucizumab (VEGF-A/ANG-2), GRB-1342 (CD38/CD3), GEM-333 (CD3/CD33), IMM-0306 (CD47/CD20);
anti-delta-like protein ligand 3 (DDL3) antibodies, such as rovalpituzumab tesirine;
anti-clusterin antibodies, such as AB-16B5;
anti-Ephrin-A4 (EFNA4) antibodies, such as PF-06647263;
anti-RANKL antibodies, such as denosumab;
anti-mesothelin antibodies, such as BMS-986148, Anti-MSLN-MMAE;
anti-sodium phosphate cotransporter 2B (NaP2B) antibodies, such as lifastuzumab;
anti-c-Met antibodies, such as ABBV-399;
Adenosine A2A receptor antagonists, such as CPI-444, AZD-4635, preladenant, PBF-509;
Dual adenosine A2A/A2B receptor antagonists, such as AB-928;
Alpha-ketoglutarate dehydrogenase (KGDH) inhibitors, such as CPI-613;
XPO1 inhibitors, such as selinexor (KPT-330);
Isocitrate dehydrogenase 2 (IDH2) inhibitors, such as enasidenib (AG-221);
IDH1 inhibitors such as AG-120, and AG-881 (IDH1 and IDH2), IDH-305, BAY-1436032; interleukin-3 receptor (IL-3R) modulators, such as SL-401;
Arginine deiminase stimulators, such as pegargiminase (ADI-PEG-20);
antibody-drug conjugates, such as MLN0264 (anti-GCC, guanylyl cyclase C), T-DM1 (trastuzumab emtansine, Kadcycla); SYD985 (anti-HER2, Duocarmycin), milatuzumab-doxorubicin (hCD74-DOX), brentuximab vedotin, DCDT2980S, polatuzumab vedotin (RG-7596), SGN-CD70A, SGN-CD19A, inotuzumab ozogamicin (CMC-544), lorvotuzumab mertansine, SAR3419, isactuzumab govitecan, enfortumab vedotin (ASG-22ME), ASG-15ME, DS-8201 ((trastuzumab deruxtecan), 225Ac-lintuzumab, U3-1402, 177Lu-tetraxetan-tetuloma, tisotumab vedotin, anetumab ravtansine, CX-2009, SAR-566658, W-0101, polatuzumab vedotin, ABBV-085, gemtuzumab ozogamicin, ABT-414, glembatumumab vedotin (CDX-011), labetuzumab govitecan (IMMU-130), sacituzumab govitecan (IMMU-132), lifastuzumab vedotin, (RG-7599), milatuzumab-doxorubicin (IMMU-110), indatuximab ravtansine (BT-062), pinatuzumab vedotin (RG-7593), SGN-LIV1A, SGN-CD33A, SAR566658, MLN2704, SAR408701, rovalpituzumab tesirine, ABBV-399, AGS-16C3F, ASG-22ME, AGS67E, AMG 172, AMG 595, AGS-15E, BAY1129980, BAY1187982, BAY94-934 (anetumab ravtansine), GSK2857916, Humax-TF-ADC (tisotumab vedotin), IMGN289, IMGN529, IMGN853 (mirvetuximab soravtansine), LOP628, PCA062, MDX-1203, MEDI-547, PF-06263507, PF-06647020, PF-06647263, PF-06664178, RG7450, RG7458, RG7598, SAR566658, SGN-CD33A, DS-1602 and DS-7300, DS-6157, DS-6000;
claudin-18 inhibitors, such as claudiximab;
β-catenin inhibitors, such as CWP-291;
anti-CD73 antibodies, such as MEDI-9447 (oleclumab), CPX-006, IPH-53, BMS-986179, NZV-930, GS-1423 (AGEN-1423);
CD73 inhibitors, such as AB-680, PSB-12379, PSB-12441, PSB-12425, CB-708, GS-1423 (AGEN-1423);
CD39/CD73 inhibitors, such as PBF-1662;
anti-CD39 antibodies, such as TTX-030;
chemokine receptor 2 (CCR) inhibitors, such as PF-04136309, CCX-872, BMS-813160 (CCR2/CCR5);
thymidylate synthase inhibitors, such as ONX-0801;
ALK/ROS1 inhibtors, such as lorlatinib;
tankyrase inhibitors, such as G007-LK;
Mdm2 p53-binding protein inhibitors, such as CMG-097, HDM-201;
c-PIM inhibitors, such as PIM447;
BRAF inhibitors, such as dabrafenib, vemurafenib, encorafenib (LGX818), PLX8394;
sphingosine kinase-2 (SK2) inhibitors, such as Yeliva® (ABC294640);
cell cycle inhibitors, such as selumetinib (MEK1/2), and sapacitabine;
AKT inhibitors such as MK-2206, ipatasertib, afuresertib, AZD5363, and ARQ-092, capivasertib, triciribine;
c-MET inhibitors, such as AMG-337, savolitinib, tivantinib (ARQ-197), capmatinib, and tepotinib, ABT-700, AG213, AMG-208, JNJ-38877618 (OMO-1), merestinib, HQP-8361;
c-Met/VEGFR inhibitors, such as BMS-817378, TAS-115;
c-Met/RON inhibitors, such as BMS-777607;
BRAF/EGFR inhibitors, such as BGB-283;
bcr/abl inhibitors, such as rebastinib, asciminib;
MNK1/MNK2 inhibitors, such as eFT-508;
mTOR inhibitor/cytochrome P450 3A4 stimulators, such as TYME-88;
lysine-specific demethylase-1 (LSD1) inhibitors, such as CC-90011;
Pan-RAF inhibitors, such as LY3009120, LXH254, TAK-580;
Raf/MEK inhibitors, such as RG7304;
CSF1R/KIT and FLT3 inhibitors, such as pexidartinib (PLX3397);
kinase inhibitors, such as vandetanib;
E selectin antagonists, such as GMI-1271;
differentiation inducers, such as tretinoin;
epidermal growth factor receptor (EGFR) inhibitors, such as osimertinib (AZD-9291);
topoisomerase inhibitors, such as doxorubicin, daunorubicin, dactinomycin, eniposide, epirubicin, etoposide, idarubicin, irinotecan, mitoxantrone, pixantrone, sobuzoxane, topotecan, irinotecan, MM-398 (liposomal irinotecan), vosaroxin and GPX-150, aldoxorubicin, AR-67, mavelertinib, AST-2818, avitinib (ACEA-0010), irofulven (MGI-114);
corticosteroids, such as cortisone, dexamethasone, hydrocortisone, methylprednisolone, prednisone, prednisolone;
growth factor signal transduction kinase inhibitors;
nucleoside analogs, such as DFP-10917;
Axl inhibitors, such as BGB-324 (bemcentinib), SLC-0211;
Inhibitors of bromodomain and extraterminal motif (BET) proteins, including BRD2 (NCBI Gene ID: 6046), BRD3 (NCBI Gene ID: 8019), BRD4 (NCBI Gene ID: 23476), and bromodomain testis-specific protein (BRDT; NCBI Gene ID: 676), such as INCB-054329, INCB057643, TEN-010, AZD-5153, ABT-767, BMS-986158, CC-90010, GSK525762 (molibresib), NHWD-870, ODM-207, GSK-2820151, GSK-1210151A, ZBC246, ZBC260, ZEN3694, FT-1101, RG-6146, CC-90010, mivebresib, BI-894999, PLX-2853, PLX-51107, CPI-0610, GS-5829;
PARP inhibitors, such as olaparib, rucaparib, veliparib, talazoparib, ABT-767, BGB-290, fluzolepali (SHR-3162), niraparib (JNJ-64091742), bendamustine hydrochloride;
PARP/Tankyrase inhibitors such as 2X-121 (e-7499);
IMP-4297, SC-10914, IDX-1197, HWH-340, CK-102, simmiparib;
Proteasome inhibitors, such as ixazomib, carfilzomib (Kyprolis®), marizomib;
Glutaminase inhibitors, such as CB-839 (telaglenastat), bis-2-(5-phenylacetamido-1,3,4-thiadiazol-2-yl)ethyl sulfide (BPTES);
mitochondrial complex I inhibitors, such as metformin, phenformin;
Vaccines, such as peptide vaccine TG-01 (RAS), GALE-301, GALE-302, nelipepimut-s, SurVaxM, DSP-7888, TPIV-200, PVX-410, VXL-100, DPX-E7, ISA-101, 6MHP, OSE-2101, galinpepimut-S, SVN53-67/M57-KLH, IMU-131; bacterial vector vaccines such as CRS-207/GVAX, axalimogene filolisbac (ADXS11-001); adenovirus vector vaccines such as nadofaragene firadenovec; autologous Gp96 vaccine; dendritic cells vaccines, such as CVactm, tapuldencel-T, eltrapuldencel-T, SL-701, BSKO1TM, rocapuldencel-T (AGS-003), DCVAC, CVactm, stapuldencel-T, eltrapuldencel-T, SL-701, BSKO1TM, ADXS31-142; oncolytic vaccines such as, talimogene laherparepvec, pexastimogene devacirepvec, GL-ONC1, MG1-MA3, parvovirus H-1, ProstAtak, enadenotucirev, MG1MA3, ASN-002 (TG-1042); therapeutic vaccines, such as CVAC-301, CMP-001, CreaVax-BC, PF-06753512, VBI-1901, TG-4010, ProscaVax™; tumor cell vaccines, such as Vigil® (IND-14205), Oncoquest-L vaccine; live attenuated, recombinant, serotype 1 poliovirus vaccine, such as PVS-RIPO; Adagloxad simolenin; MEDI-0457; DPV-001 a tumor-derived, autophagosome enriched cancer vaccine; RNA vaccines such as, CV-9209, LV-305; DNA vaccines, such as MEDI-0457, MVI-816, INO-5401; modified vaccinia virus Ankara vaccine expressing p53, such as MVA-p53; DPX-Survivac; BriaVax™; GI-6301; GI-6207; GI-4000; 10-103; Neoantigen peptide vaccines, such as AGEN-2017, GEN-010, NeoVax, RG-6180, GEN-009, PGV-001 (TLR-3 agonist), GRANITE-001, NEO-PV-01; Peptide vaccines that target heat shock proteins, such as PhosphoSynVax™; Vitespen (HSPPC-96-C);
anti-DLL4 (delta like ligand 4) antibodies, such as demcizumab;
STAT-3 inhibitors, such as napabucasin (BBI-608);
ATPase p97 inhibitors, such as CB-5083;
smoothened (SMO) receptor inhibitors, such as Odomzo® (sonidegib, formerly LDE-225), LEQ506, vismodegib (GDC-0449), BMS-833923, glasdegib (PF-04449913), LY2940680, and itraconazole;
interferon alpha ligand modulators, such as interferon alpha-2b, interferon alpha-2a biosimilar (Biogenomics), ropeginterferon alfa-2b (AOP-2014, P-1101, PEG IFN alpha-2b), Multiferon (Alfanative, Viragen), interferon alpha 1b, Roferon-A (Canferon, Ro-25-3036), interferon alfa-2a follow-on biologic (Biosidus)(Inmutag, Inter 2A), interferon alfa-2b follow-on biologic (Biosidus—Bioferon, Citopheron, Ganapar, Beijing Kawin Technology—Kaferon), Alfaferone, pegylated interferon alpha-1b, peginterferon alfa-2b follow-on biologic (Amega), recombinant human interferon alpha-1b, recombinant human interferon alpha-2a, recombinant human interferon alpha-2b, veltuzumab-IFN alpha 2b conjugate, Dynavax (SD-101), and interferon alfa-n1 (Humoferon, SM-10500, Sumiferon);
interferon gamma ligand modulators, such as interferon gamma (OH-6000, Ogamma 100);
IL-6 receptor modulators, such as tocilizumab, siltuximab, AS-101 (CB-06-02, IVX-Q-101);
Telomerase modulators, such as, tertomotide (GV-1001, HR-2802, Riavax) and imetelstat (GRN-163, JNJ-63935937);
DNA methyltransferases inhibitors, such as temozolomide (CCRG-81045), decitabine, guadecitabine (S-110, SGI-110), KRX-0402, RX-3117, RRx-001, and azacitidine;
DNA gyrase inhibitors, such as pixantrone and sobuzoxane;
Bcl-2 family protein inhibitors, such as ABT-263, venetoclax (ABT-199), ABT-737, and AT-101;
Notch inhibitors, such as LY3039478 (crenigacestat), tarextumab (anti-Notch2/3), BMS-906024;
anti-myostatin inhibitors, such as landogrozumab;
hyaluronidase stimulators, such as PEGPH-20;
Wnt pathway inhibitors, such as SM-04755, PRI-724, WNT-974;
gamma-secretase inhibitors, such as PF-03084014, MK-0752, RO-4929097;
Grb-2 (growth factor receptor bound protein-2) inhibitors, such as BP1001;
TRAIL pathway-inducing compounds, such as ONC201, ABBV-621;
Focal adhesion kinase inhibitors, such as VS-4718, defactinib, GSK2256098;
hedgehog inhibitors, such as saridegib, sonidegib (LDE225), glasdegib and vismodegib;
Aurora kinase inhibitors, such as alisertib (MLN-8237), and AZD-2811, AMG-900, barasertib, ENMD-2076;
HSPB1 modulators (heat shock protein 27, HSP27), such as brivudine, apatorsen;
ATR inhibitors, such as BAY-937, AZD6738, AZD6783, VX-803, VX-970 (berzosertib) and VX-970;
mTOR inhibitors, such as sapanisertib and vistusertib (AZD2014), ME-344;
mTOR/PI3K inhibitors, such as gedatolisib, GSK2141795, omipalisib, RG6114;
Hsp90 inhibitors, such as AUY922, onalespib (AT13387), SNX-2112, SNX5422;
Murine double minute (mdm2) oncogene inhibitors, such as DS-3032b, RG7775, AMG-232, HDM201, and idasanutlin (RG7388);
CD137 agonists, such as urelumab, utomilumab (PF-05082566), AGEN2373, ADG-106; STING agonists, such as ADU-S100 (MIW-815), SB-11285, MK-1454, SR-8291, AdVCA0848, GSK-532, SYN-STING, MSA-1, SR-8291;
FGFR inhibitors, such as FGF-401, INCB-054828, BAY-1163877, AZD4547, JNJ-42756493, LY2874455, Debio-1347;
fatty acid synthase (FASN) inhibitors, such as TVB-2640;
Anti-killer cell immunoglobulin like receptor, three Ig domains and long cytoplasmic tail 1 (KIR3DL1; KIR; NCBI Gene ID: 3811) monoclonal antibodies, such as lirilumab (IPH-2102), IPH-4102;
Antigen CD19 inhibitors, such as MOR208, MEDI-551, AFM-11, inebilizumab;
CD44 binders, such as A6;
protein phosphatease 2A (PP2A) inhibitors, such as LB-100;
CYP17 inhibitors, such as seviteronel (VT-464), ASN-001, ODM-204, CFG920, abiraterone acetate;
RXR agonists, such as IRX4204;
hedgehog/smoothened (hh/Smo) antagonists, such as taladegib, patidegib;
complement C3 modulators, such as Imprime PGG;
IL-15 agonists, such as ALT-803, NKTR-255, and hetlL-15;
EZH2 (enhancer of zeste homolog 2) inhibitors, such as tazemetostat, CPI-1205, GSK-2816126;
Oncolytic viruses, such as pelareorep, CG-0070, MV-NIS therapy, HSV-1716, DS-1647, VCN-01, ONCOS-102, TBI-1401, tasadenoturev (DNX-2401), vocimagene amiretrorepvec, RP-1, CVA21, Celyvir, LOAd-703, OBP-301;
DOT1L (histone methyltransferase) inhibitors, such as pinometostat (EPZ-5676);
toxins such as Cholera toxin, ricin, Pseudomonas exotoxin, Bordetella pertussis adenylate cyclase toxin, diphtheria toxin, and caspase activators;
DNA plasmids, such as BC-819;
PLK inhibitors of PLK 1, 2, and 3, such as volasertib (PLK1);
WEE1 inhibitors, such as AZD-1775 (adavosertib); Rho kinase (ROCK) inhibitors, such as AT13148, KD025;
ERK inhibitors, such as GDC-0994, LY3214996, MK-8353;
Inhibition of Apoptosis Protein (IAP) inhibitors, such as ASTX660, debio-1143, birinapant, APG-1387, LCL-161;
RNA polymerase inhibitors, such has lurbinectedin (PM-1183), CX-5461;
Tubulin inhibitors, such as PM-184, BAL-101553 (lisavanbulin), and OXI-4503, fluorapacin (AC-0001), plinabulin;
Toll-like receptor 4 (TL4) agonists, such as G100, GSK1795091, and PEPA-10;
Elongation factor 1 alpha 2 inhibitors, such as plitidepsin;
CD95 inhibitors, such as APG-101, APO-010, asunercept;
WT1 inhibitors, such as DSP-7888;
splicing factor 3B subunit1 (SF3B1) inhibitors, such as H3B-8800;
retinoid Z receptor gamma (RORγ) agonists, such as LYC-55716; and
Microbiome modulators, such as SER-401, EDP-1503, MRx-0518.

In some embodiments, the fusion protein, the homodimer, the heterodimer, the conjugate, the polynucleotide, the vector, the lipoplex, such as an LNP, and/or the pharmaceutical composition is co-administered with one or more additional therapeutic agents comprising an inhibitor or antagonist of: protein tyrosine phosphatase, non-receptor type 11 (PTPN11 or SHP2; NCBI Gene ID: 5781); myeloid cell leukemia sequence 1 (MCL1) apoptosis regulator (NCBI Gene ID: 4170); mitogen-activated protein kinase kinase kinase kinase 1 (MAP4K1) (also called Hematopoietic Progenitor Kinase 1 (HPK1), NCBI Gene ID: 11184); phosphatidylinositol-4,5-bisphosphate 3-kinase, including catalytic subunit alpha (PIK3CA; NCBI Gene ID: 5290), catalytic subunit beta (PIK3CB; NCBI Gene ID: 5291), catalytic subunit gamma (PIK3CG; NCBI Gene ID: 5294) and catalytic subunit delta (PIK3CD; NCBI Gene ID: 5293), diacylglycerol kinase alpha (DGKA, DAGK, DAGK1 or DGK-alpha; NCBI Gene ID: 1606); 5′-nucleotidase ecto (NTSE or CD73; NCBI Gene ID: 4907); ectonucleoside triphosphate diphosphohydrolase 1 (ENTPD1 or CD39; NCBI Gene ID: 593); transforming growth factor beta 1 (TGFB1 or TGFβ; NCBI Gene ID: 7040); heme oxygenase 1 (HMOX1, HO-1 or H01; NCBI Gene ID: 3162); heme oxygenase 2 (HMOX2, HO-2 or H02; NCBI Gene ID: 3163); vascular endothelial growth factor A (VEGFA or VEGF; NCBI Gene ID: 7422); erb-b2 receptor tyrosine kinase 2 (ERBB2, HER2, HER2/neu or CD340; NCBI Gene ID: 2064), epidermal growth factor receptor (EGFR, ERBB, ERBB1 or HER1; NCBI Gene ID: 1956); ALK receptor tyrosine kinase (ALK, CD246; NCBI Gene ID: 238); poly(ADP-ribose) polymerase 1 (PARP1; NCBI Gene ID: 142); poly(ADP-ribose) polymerase 2 (PARP2; NCBI Gene ID: 10038); TCDD inducible poly(ADP-ribose) polymerase (TIPARP, PARP7; NCBI Gene ID: 25976); cyclin dependent kinase 4 (CDK4; NCBI Gene ID: 1019); cyclin dependent kinase 6 (CDK6; NCBI Gene ID: 1021); TNF receptor superfamily member 14 (TNFRSF14, HVEM, CD270; NCBI Gene ID: 8764); T cell immunoreceptor with Ig and ITIM domains (TIGIT; NCBI Gene ID: 201633); X-linked inhibitor of apoptosis (XIAP, BIRC4, IAP-3; NCBI Gene ID: 331); baculoviral IAP repeat containing 2 (BIRC2, cIAP1; NCBI Gene ID: 329); baculoviral IAP repeat containing 3 (BIRC3, cIAP2; NCBI Gene ID: 330); baculoviral IAP repeat containing 5 (BIRC5, surviving; NCBI Gene ID: 332); C-C motif chemokine receptor 2 (CCR2, CD192; NCBI Gene ID: 729230); C-C motif chemokine receptor 5 (CCR5, CD195; NCBI Gene ID: 1234); C-C motif chemokine receptor 8 (CCR8, CDw198; NCBI Gene ID: 1237); C-X-C motif chemokine receptor 2 (CXCR2, CD182; NCBI Gene ID: 3579); C-X-C motif chemokine receptor 3 (CXCR3, CD182, CD183; NCBI Gene ID: 2833); C-X-C motif chemokine receptor 4 (CXCR4, CD184; NCBI Gene ID: 7852); cytokine inducible SH2 containing protein (CISH; NCBI Gene ID: 1154); arginase (ARG1 (NCBI Gene ID: 383), ARG2 (NCBI Gene ID: 384)), carbonic anhydrase (CA1 (NCBI Gene ID: 759), CA2 (NCBI Gene ID: 760), CA3 (NCBI Gene ID: 761), CA4 (NCBI Gene ID: 762), CASA (NCBI Gene ID: 763), CA5B (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CA11 (NCBI Gene ID: 770), CA12 (NCBI Gene ID: 771), CA13 (NCBI Gene ID: 377677), CA14 (NCBI Gene ID: 23632)), prostaglandin-endoperoxide synthase 1 (PTGS1, COX-1; NCBI Gene ID: 5742), prostaglandin-endoperoxide synthase 2 (PTGS2, COX-2; NCBI Gene ID: 5743), secreted phospholipase A2, prostaglandin E synthase (PTGES, PGES; Gene ID: 9536), arachidonate 5-lipoxygenase (ALOX5, 5-LOX; NCBI Gene ID: 240) and/or soluble epoxide hydrolase 2 (EPHX2, SEH; NCBI Gene ID: 2053); a secreted phospholipase A2 (e.g., PLA2G1B (NCBI Gene ID: 5319); PLA2G7 (NCBI Gene ID: 7941), PLA2G3 (NCBI Gene ID: 50487), PLA2G2A (NCBI Gene ID: 5320); PLA2G4A (NCBI Gene ID: 5321); PLA2G12A (NCBI Gene ID: 81579); PLA2G12B (NCBI Gene ID: 84647); PLA2G10 (NCBI Gene ID: 8399); PLA2G5 (NCBI Gene ID: 5322); PLA2G2D (NCBI Gene ID: 26279); PLA2G15 (NCBI Gene ID: 23659)); indoleamine 2,3-dioxygenase 1 (IDO1; NCBI Gene ID: 3620); indoleamine 2,3-dioxygenase 2 (IDO2; NCBI Gene ID: 169355); hypoxia inducible factor 1 subunit alpha (HIF1A; NCBI Gene ID: 3091); angiopoietin 1 (ANGPT1; NCBI Gene ID: 284); Endothelial TEK tyrosine kinase (TIE-2, TEK, CD202B; NCBI Gene ID: 7010); Janus kinase 1 (JAK1; NCBI Gene ID: 3716); catenin beta 1 (CTNNB1; NCBI Gene ID: 1499); histone deacetylase 9 (HDAC9; NCBI Gene ID: 9734), 5′-3′ exoribonuclease 1 (XRN1; NCBI Gene ID: 54464); and/or WRN RecQ like helicase (WRN; NCBI Gene ID: 7486).

### Immune Checkpoint Modulators

In various embodiments, the FLT3L-Fc fusion proteins, homodimers, heterodimers, polynucleotides, vectors, lipoplexes, such as LNPs, and/or pharmaceutical compositions, as described herein, are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of cancer cells within the tumor microenvironment. Activation or stimulation of stimulatory immune check points can augment the effect of immune checkpoint inhibitors in cancer therapeutics. In various embodiments, the immune checkpoint proteins or receptors regulate T cell responses (e.g., reviewed in Xu, et al., J Exp Clin Cancer Res. (2018) 37:110). In various embodiments, the immune checkpoint proteins or receptors regulate NK cell responses (e.g., reviewed in Davis, et al., Semin Immunol. (2017) 31:64-75 and Chiossone, et al., Nat Rev Immunol. (2018) 18(11):671-688).

Examples of immune checkpoint proteins or receptors include without limitation CD27 (NCBI Gene ID: 939), CD70 (NCBI Gene ID: 970); CD40 (NCBI Gene ID: 958), CD40LG (NCBI Gene ID: 959); CD47 (NCBI Gene ID: 961), SIRPA (NCBI Gene ID: 140885); CD48 (SLAMF2; NCBI Gene ID: 962), transmembrane and immunoglobulin domain containing 2 (TMIGD2, CD28H; NCBI Gene ID: 126259), CD84 (LY9B, SLAMF5; NCBI Gene ID: 8832), CD96 (NCBI Gene ID: 10225), CD160 (NCBI Gene ID: 11126), MS4A1 (CD20; NCBI Gene ID: 931), CD244 (SLAMF4; NCBI Gene ID: 51744); CD276 (B7H3; NCBI Gene ID: 80381); V-set domain containing T cell activation inhibitor 1 (VTCN1, B7H4); V-set immunoregulatory receptor (VSIR, B7H5, VISTA; NCBI Gene ID: 64115); immunoglobulin superfamily member 11 (IGSF11, VSIG3; NCBI Gene ID: 152404); natural killer cell cytotoxicity receptor 3 ligand 1 (NCR3LG1, B7H6; NCBI Gene ID: 374383); HERV-H LTR-associating 2 (HHLA2, B7H7; NCBI Gene ID: 11148); inducible T cell co-stimulator (ICOS, CD278; NCBI Gene ID: 29851); inducible T cell co-stimulator ligand (ICOSLG, B7H2; NCBI Gene ID: 23308); TNF receptor superfamily member 4 (TNFRSF4, OX40; NCBI Gene ID: 7293); TNF superfamily member 4 (TNFSF4, OX40L; NCBI Gene ID: 7292); TNFRSF8 (CD30; NCBI Gene ID: 943), TNFSF8 (CD30L; NCBI Gene ID: 944); TNFRSF10A (CD261, DR4, TRAILR1; NCBI Gene ID: 8797), TNFRSF9 (CD137; NCBI Gene ID: 3604), TNFSF9 (CD137L; NCBI Gene ID: 8744); TNFRSF10B (CD262, DR5, TRAILR2; NCBI Gene ID: 8795), TNFRSF10 (TRAIL; NCBI Gene ID: 8743); TNFRSF14 (HVEM, CD270; NCBI Gene ID: 8764), TNFSF14 (HVEML; NCBI Gene ID: 8740); CD272 (B and T lymphocyte associated (BTLA); NCBI Gene ID: 151888); TNFRSF17 (BCMA, CD269; NCBI Gene ID: 608), TNFSF13B (BAFF; NCBI Gene ID: 10673); TNFRSF18 (GITR; NCBI Gene ID: 8784), TNFSF18 (GITRL; NCBI Gene ID: 8995); MHC class I polypeptide-related sequence A (MICA; NCBI Gene ID: 100507436); MHC class I polypeptide-related sequence B (MICB; NCBI Gene ID: 4277); CD274 (CD274, PDL1, PD-L1; NCBI Gene ID: 29126); programmed cell death 1 (PDCD1, PD1, PD-1; NCBI Gene ID: 5133); cytotoxic T-lymphocyte associated protein 4 (CTLA4, CD152; NCBI Gene ID: 1493); CD80 (B7-1; NCBI Gene ID: 941), CD28 (NCBI Gene ID: 940); nectin cell adhesion molecule 2 (NECTIN2, CD112; NCBI Gene ID: 5819); CD226 (DNAM-1; NCBI Gene ID: 10666); Poliovirus receptor (PVR) cell adhesion molecule (PVR, CD155; NCBI Gene ID: 5817); PVR related immunoglobulin domain containing (PVRIG, CD112R; NCBI Gene ID: 79037); T cell immunoreceptor with Ig and ITIM domains (TIGIT; NCBI Gene ID: 201633); T cell immunoglobulin and mucin domain containing 4 (TIMD4; TIM4; NCBI Gene ID: 91937); hepatitis A virus cellular receptor 2 (HAVCR2, TIMD3, TIM3; NCBI Gene ID: 84868); galectin 9 (LGALS9; NCBI Gene ID: 3965); lymphocyte activating 3 (LAG3, CD223; NCBI Gene ID: 3902); signaling lymphocytic activation molecule family member 1 (SLAMF1, SLAM, CD150; NCBI Gene ID: 6504); lymphocyte antigen 9 (LY9, CD229, SLAMF3; NCBI Gene ID: 4063); SLAM family member 6 (SLAMF6, CD352; NCBI Gene ID: 114836); SLAM family member 7 (SLAMF7, CD319; NCBI Gene ID: 57823); UL16 binding protein 1 (ULBP1; NCBI Gene ID: 80329); UL16 binding protein 2 (ULBP2; NCBI Gene ID: 80328); UL16 binding protein 3 (ULBP3; NCBI Gene ID: 79465); retinoic acid early transcript 1E (RAET1E; ULBP4; NCBI Gene ID: 135250); retinoic acid early transcript 1G (RAET1G; ULBP5; NCBI Gene ID: 353091); retinoic acid early transcript 1L (RAET1L; ULBP6; NCBI Gene ID: 154064); killer cell immunoglobulin like receptor, three Ig domains and long cytoplasmic tail 1 (KIR, CD158E1; NCBI Gene ID: 3811, e.g., lirilumab (IPH-2102, IPH-4102)); killer cell lectin like receptor C1 (KLRC1, NKG2A, CD159A; NCBI Gene ID: 3821); killer cell lectin like receptor K1 (KLRK1, NKG2D, CD314; NCBI Gene ID: 22914); killer cell lectin like receptor C2 (KLRC2, CD159c, NKG2C; NCBI Gene ID: 3822); killer cell lectin like receptor C3 (KLRC3, NKG2E; NCBI Gene ID: 3823); killer cell lectin like receptor C4 (KLRC4, NKG2F; NCBI Gene ID: 8302); killer cell immunoglobulin like receptor, two Ig domains and long cytoplasmic tail 1 (KIR2DL1; NCBI Gene ID: 3802); killer cell immunoglobulin like receptor, two Ig domains and long cytoplasmic tail 2 (KIR2DL2; NCBI Gene ID: 3803); killer cell immunoglobulin like receptor, two Ig domains and long cytoplasmic tail 3 (KIR2DL3; NCBI Gene ID: 3804); killer cell immunoglobulin like receptor, three Ig domains and long cytoplasmic tail 1 (KIR3DL1); killer cell lectin like receptor D1 (KLRD1; NCBI Gene ID: 3824); killer cell lectin like receptor G1 (KLRG1; CLEC15A, MAFA, 2F1; NCBI Gene ID: 10219); sialic acid binding Ig like lectin 7 (SIGLEC7; NCBI Gene ID: 27036); and sialic acid binding Ig like lectin 9 (SIGLEC9; NCBI Gene ID: 27180).

In various embodiments, the FLT3L-Fc fusion proteins, homodimers, heterodimers, polynucleotides, vectors, lipoplexes, such as LNPs, and/or pharmaceutical compositions, as described herein, are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the FLT3L-Fc fusion proteins, homodimers, heterodimers, polynucleotides, vectors, lipoplexes, such as LNPs, and/or pharmaceutical compositions, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu, et al., J Exp Clin Cancer Res. (2018) 37:110.

In various embodiments, the FLT3L-Fc fusion proteins, homodimers, heterodimers, polynucleotides, vectors, lipoplexes, such as LNPs, and/or pharmaceutical compositions, as described herein, are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); killer cell lectin like receptor D1 (KLRD1, CD94), killer cell lectin like receptor G1 (KLRG1; CLEC15A, MAFA, 2F1); sialic acid binding Ig like lectin 7 (SIGLEC7); and sialic acid binding Ig like lectin 9 (SIGLEC9). In various embodiments, the FLT3L-Fc fusion proteins, homodimers, heterodimers, polynucleotides, vectors, lipoplexes, such as LNPs, and/or pharmaceutical compositions, as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis, et al., Semin Immunol. (2017) 31:64-75; Fang, et al., Semin Immunol. (2017) 31:37-54; and Chiossone, et al., Nat Rev Immunol. (2018) 18(11):671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4.

Examples of inhibitors of CTLA4 that can be co-administered include without limitation ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, AGEN2034 (balstilimab), zimberelimab, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

### TNF Receptor Superfamily (TNFRSF) Member Agonists or Activators

In various embodiments, the FLT3L-Fc fusion proteins, homodimers, heterodimers, polynucleotides, vectors, lipoplexes, such as LNPs, and/or pharmaceutical compositions, as described herein, are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Example anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Example anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Example anti-TNFRSF9 (4-1BB, CD137) antibodies that can be co-administered include without limitation urelumab, utomilumab (PF-05082566), AGEN2373 and ADG-106.

In some embodiments, the anti-TNFRSF17 (BCMA) antibody GSK-2857916 is co-administered.

Example anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

Bi-specific antibodies targeting TNFRSF family members that can be co-administered include without limitation PRS-343 (CD-137/HER2), AFM26 (BCMA/CD16A), AFM-13 (CD16/CD30), REGN-1979 (CD20/CD3), AMG-420 (BCMA/CD3), INHIBRX-105 (4-1BB/PDL1), FAP-4-IBBL (4-1BB/FAP), XmAb-13676 (CD3/CD20), RG-7828 (CD20/CD3), CC-93269 (CD3/BCMA), REGN-5458 (CD3/BCMA), and IMM-0306 (CD47/CD20).

### Bi-Specific T-Cell Engagers

In various embodiments, the FLT3L-Fc fusion proteins, homodimers, heterodimers, polynucleotides, vectors, lipoplexes, such as LNPs, and/or pharmaceutical compositions, as described herein, are combined with a bi-specific T-cell engager (e.g., not having an Fc) or an anti-CD3 bi-specific antibody (e.g., having an Fc). Illustrative anti-CD3 bi-specific antibodies or BiTEs that can be co-administered include JNJ-64052781 (CD19/CD3), AMG-211 (CEA/CD3), RG7802 (CEA/CD3), ERY-974 (CD3/GPC3), PF-06671008 (Cadherins/CD3), APV0436 (CD123/CD3), flotetuzumab (CD123/CD3), REGN-1979 (CD20/CD3), MCLA-117 (CD3/CLEC12A), JNJ-0819, JNJ-7564 (CD3/heme), AMG-757 (DLL3-CD3), AMG-330 (CD33/CD3), AMG-420 (BCMA/CD3), JNJ-63709178 (CD123/CD3), MGD-007 (CD3/gpA33), MGD-009 (CD3/B7H3), IMCgp100 (CD3/gp100), XmAb-14045 (CD123/CD3), XmAb-13676 (CD3/CD20), XmAb-18087 (SSTR2/CD3), catumaxomab (CD3/EpCAM), REGN-4018 (MUC16/CD3), RG-7828 (CD20/CD3), CC-93269 (CD3/BCMA), REGN-5458 (CD3/BCMA), GRB-1302 (CD3/Erbb2), GRB-1342 (CD38/CD3), GEM-333 (CD3/CD33). As appropriate, the anti-CD3 binding bi-specific molecules may or may not have an Fc. Illustrative bi-specific T-cell engagers that can be co-administered target CD3 and a tumor-associated antigen as described herein, including, e.g., CD19 (e.g., blinatumomab); CD33 (e.g., AMG330); CEA (e.g., MEDI-565); receptor tyrosine kinase-like orphan receptor 1 (ROR1) (Gohil, et al., Oncoimmunology. (2017) May 17; 6(7):e1326437); PD-L1 (Horn, et al., Oncotarget. 2017 Aug. 3; 8(35):57964-57980); and EGFRvIII (Yang, et al., Cancer Lett. 2017 Sep. 10; 403:224-230).

### Bi- and Tri-Specific Natural Killer (NK)-Cell Engagers

In various embodiments, the FLT3L-Fc fusion proteins, homodimers, heterodimers, polynucleotides, vectors, lipoplexes, such as LNPs, and/or pharmaceutical compositions, as described herein, are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). Illustrative anti-CD16 bi-specific antibodies, BiKEs or TriKEs that can be co-administered include AFM26 (BCMA/CD16A) and AFM-13 (CD16/CD30). As appropriate, the anti-CD16 binding bi-specific molecules may or may not have an Fc. Illustrative bi-specific NK-cell engagers that can be co-administered target CD16 and one or more tumor-associated antigens as described herein, including, e.g., CD19, CD20, CD22, CD30, CD33, CD123, EGFR, EpCAM, ganglioside GD2, HER2/neu, HLA Class II and FOLR1. BiKEs and TriKEs are described, e.g., in Felices, et al., Methods Mol Biol. (2016) 1441:333-346; Fang, et al., Semin Immunol. (2017) 31:37-54.

### MCL1 Apoptosis Regulator, BCL2 Family Member (MCL1) Inhibitors

In various embodiments, the FLT3L-Fc fusion proteins, homodimers, heterodimers, polynucleotides, vectors, lipoplexes, such as LNPs, and/or pharmaceutical compositions, as described herein, are combined with an inhibitor of MCL1 apoptosis regulator, BCL2 family member (MCL1, TM; EAT; MCL1L; MCL1S; Mc1-1; BCL2L3; MCL1-ES; bc12-L-3; mcl1/EAT; NCBI Gene ID: 4170). Examples of MCL1 inhibitors include GS-9716, AMG-176, AMG-397, S-64315, and AZD-5991, 483-LM, A-1210477, UMI-77, JKY-5-037, and those described in WO 2019/222112, WO2018183418, WO2016033486, and WO2017147410.

### SHP2 Inhibitors

In various embodiments, the FLT3L-Fc fusion proteins, homodimers, heterodimers, polynucleotides, vectors, lipoplexes, such as LNPs, and/or pharmaceutical compositions, as described herein, are combined with an inhibitor of protein tyrosine phosphatase non-receptor type 11 (PTPN11; BPTP3, CFC, JMML, METCDS, NS1, PTP-1D, PTP2C, SH-PTP2, SH-PTP3, SHP2; NCBI Gene ID: 5781). Examples of SHP2 inhibitors include TNO155 (SHP-099), RMC-4550, JAB-3068, RMC-4630, and those described in WO2018172984 and WO2017211303.

### Hematopoietic Progenitor Kinase 1 (HPK1) Inhibitors

In various embodiments, the FLT3L-Fc fusion proteins, homodimers, heterodimers, polynucleotides, vectors, lipoplexes, such as LNPs, and/or pharmaceutical compositions, as described herein, are combined with an inhibitor of mitogen-activated protein kinase kinase kinase kinase 1 (MAP4K1, HPK1; NCBI Gene ID: 11184). Examples of Hematopoietic Progenitor Kinase 1 (HPK1) inhibitors include without limitation, those described in WO-2018183956, WO-2018183964, WO-2018167147, WO-2018183964, WO-2016205942, WO-2018049214, WO-2018049200, WO-2018049191, WO-2018102366, WO-2018049152 and WO-2016090300;

### Apoptosis Signal-Regulating Kinase (ASK) Inhibitors

In various embodiments, the FLT3L-Fc fusion proteins, homodimers, heterodimers, polynucleotides, vectors, lipoplexes, such as LNPs, and/or pharmaceutical compositions, as described herein, are combined with an inhibitor of an ASK inhibitor, e.g., mitogen-activated protein kinase kinase kinase 5 (MAP3K5; ASK1, MAPKKK5, MEKK5; NCBI Gene ID: 4217). Examples of ASK1 inhibitors include without limitation, those described in WO 2011/008709 (Gilead Sciences) and WO 2013/112741 (Gilead Sciences).

### Bruton Tyrosine Kinase (BTK) Inhibitors

In various embodiments, the FLT3L-Fc fusion proteins, homodimers, heterodimers, polynucleotides, vectors, lipoplex, such as LNPs, and/or pharmaceutical compositions, as described herein, are combined with an inhibitor of Bruton tyrosine kinase (BTK, AGMX1, AT, ATK, BPK, IGHD3, IMD1, PSCTK1, XLA; NCBI Gene ID: 695). Examples of BTK inhibitors include without limitation, (S)-6-amino-9-(1-(but-2-ynoyl)pyrrolidin-3-yl)-7-(4-phenoxyphenyl)-7H-purin-8(9H)-one, acalabrutinib (ACP-196), BGB-3111, CB988, HM71224, ibrutinib, M-2951 (evobrutinib), M7583, tirabrutinib (ONO-4059), PRN-1008, spebrutinib (CC-292), TAK-020, vecabrutinib, ARQ-531, SHR-1459, DTRMWXHS-12, TAS-5315.

### Cluster of Differentiation 47 (CD47) Inhibitors

In various embodiments, the FLT3L-Fc fusion proteins, homodimers, heterodimers, polynucleotides, vectors, lipoplex, such as LNPs, and/or pharmaceutical compositions, as described herein, are combined with an inhibitor of CD47 (IAP, MER6, 0A3; NCBI Gene ID: 961). Examples of CD47 inhibitors include without limitation anti-CD47 mAbs (Vx-1004), anti-human CD47 mAbs (CNTO-7108), CC-90002, CC-90002-ST-001, humanized anti-CD47 antibody (Hu5F9-G4), NI-1701, NI-1801, RCT-1938, and TTI-621. In some embodiments, the CD47 inhibitor is magrolimab.

### SIRPA (SIRPα) Targeting Agents

In various embodiments, the FLT3L-Fc fusion proteins, homodimers, heterodimers, polynucleotides, vectors, lipoplexes, such as LNPs, and/or pharmaceutical compositions, as described herein, are combined with a SIRPα targeting agent (NCBI Gene ID: 140885; UniProt P78324). Examples of SIRPα targeting agents include without limitation SIRPα inhibitors, such as AL-008, RRx-001, and CTX-5861, and anti-SIRPα antibodies, such as FSI-189 (GS-0189), ES-004, BI765063, ADU1805, and CC-95251. Additional SIRPα-targeting agents of use are described, for example, in WO200140307, WO2002092784, WO2007133811, WO2009046541, WO2010083253, WO2011076781, WO2013056352, WO2015138600, WO2016179399, WO2016205042, WO2017178653, WO2018026600, WO2018057669, WO2018107058, WO2018190719, WO2018210793, WO2019023347, WO2019042470, WO2019175218, WO2019183266, WO2020013170 and WO2020068752.

### Cyclin-Dependent Kinase (CDK) Inhibitors

In various embodiments, the FLT3L-Fc fusion proteins, homodimers, heterodimers, polynucleotides, vectors, lipoplexes, such as LNPs, and/or pharmaceutical compositions, as described herein, are combined with an inhibitor of cyclin dependent kinase 1 (CDK1, CDC2; CDC28A; P34CDC2; NCBI Gene ID: 983); cyclin dependent kinase 2 (CDK2, CDKN2; p33(CDK2); NCBI Gene ID: 1017); cyclin dependent kinase 3 (CDK3, ; NCBI Gene ID: 1018); cyclin dependent kinase 4 (CDK4, CMM3; PSK-J3; NCBI Gene ID: 1019); cyclin dependent kinase 6 (CDK6, MCPH12; PLSTIRE; NCBI Gene ID: 1021); cyclin dependent kinase 7 (CDK7, CAK; CAK1; HCAK; MO15; STK1; CDKN7; p39MO15; NCBI Gene ID: 1022); cyclin dependent kinase 9 (CDK9, TAK; C-2k; CTK1; CDC2L4; PITALRE; NCBI Gene ID: 1025). Inhibitors of CDK 1, 2, 3, 4, 6, 7 and/or 9, include without limitation abemaciclib, alvocidib (HMR-1275, flavopiridol), AT-7519, dinaciclib, ibrance, FLX-925, LEE001, palbociclib, ribociclib, rigosertib, selinexor, UCN-01, SY1365, CT-7001, SY-1365, G1T38, milciclib, trilaciclib, and TG-02.

### Discoidin Domain Receptor (DDR) Inhibitors

In various embodiments, the FLT3L-Fc fusion proteins, homodimers, heterodimers, polynucleotides, vectors, lipoplexes, such as LNPs, and/or pharmaceutical compositions, as described herein, are combined with an inhibitor of discoidin domain receptor tyrosine kinase 1 (DDR1, CAK, CD167, DDR, EDDR1, HGK2, MCK10, NEP, NTRK4, PTK3, PTK3A, RTK6, TRKE; NCBI Gene ID: 780); and/or discoidin domain receptor tyrosine kinase 2 (DDR2, MIG20a, NTRKR3, TKT, TYRO10, WRCN; NCBI Gene ID: 4921). Examples of DDR inhibitors include without limitation, dasatinib and those disclosed in WO2014/047624 (Gilead Sciences), US 2009-0142345 (Takeda Pharmaceutical), US 2011-0287011 (Oncomed Pharmaceuticals), WO 2013/027802 (Chugai Pharmaceutical), and WO2013/034933 (Imperial Innovations).

### Targeted E3 Ligase Ligand Conjugates

In various embodiments, the FLT3L-Fc fusion proteins, homodimers, heterodimers, polynucleotides, vectors, lipoplexes, such as LNPs, and/or pharmaceutical compositions, as described herein, are combined with a targeted E3 ligase ligand conjugate. Such conjugates have a target protein binding moiety and an E3 ligase binding moiety (e.g., an inhibitor of apoptosis protein (IAP) (e.g., XIAP, c-IAP1, c-IAP2, NIL-IAP, Bruce, and surviving) E3 ubiquitin ligase binding moiety, Von Hippel-Lindau E3 ubiquitin ligase (VHL) binding moiety, a cereblon E3 ubiquitin ligase binding moiety, mouse double minute 2 homolog (MDM2) E3 ubiquitin ligase binding moiety), and can be used to promote or increase the degradation of targeted proteins, e.g., via the ubiquitin pathway. In one embodiment, the targeted E3 ligase ligand conjugates comprise a targeting or binding moiety that targets or binds a protein identified in Table B, and an E3 ligase ligand or binding moiety. In one embodiment, the targeted E3 ligase ligand conjugates comprise a targeting or binding moiety that targets or binds a protein selected from Cbl proto-oncogene B (CBLB; Cbl-b, Nbla00127, RNF56; NCBI Gene ID: 868) and hypoxia inducible factor 1 subunit alpha (HIF1A; NCBI Gene ID: 3091). In one embodiment, the targeted E3 ligase ligand conjugates comprise a kinase inhibitor (e.g., a small molecule kinase inhibitor, e.g., of BTK and an E3 ligase ligand or binding moiety. See, e.g., WO2018098280. In another embodiment, the targeted E3 ligase ligand conjugates comprise a binding moiety targeting or binding to Interleukin-1 (IL-1) Receptor-Associated Kinase-4 (IRAK-4); Rapidly Accelerated Fibrosarcoma (RAF, such as c-RAF, A-RAF and/or B-RAF), c-Met/p38, or a BRD protein; and an E3 ligase ligand or binding moiety. See, e.g., WO2019099926, WO2018226542, WO2018119448, WO2018223909, WO2019079701. Additional targeted E3 ligase ligand conjugates that can be co-administered are described, e.g., in WO2018237026, WO2019084026, WO2019084030, WO2019067733, WO2019043217, WO2019043208 and WO2018144649.

### Histone Deacetylase (HDAC) Inhibitors

In various embodiments, the FLT3L-Fc fusion proteins, homodimers, heterodimers, polynucleotides, vectors, lipoplexes, such as LNPs, and/or pharmaceutical compositions, as described herein, are combined with an inhibitor of a histone deacetylase, e.g., histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CUDC-907 (fimepinostat), entinostat, givinostat, mocetinostat, panobinostat, pracinostat, quisinostat (JNJ-26481585), resminostat, ricolinostat, SHP-141, valproic acid (VAL-001), vorinostat, tinostamustine, remetinostat, entinostat.

Indoleamine-pyrrole-2,3-dioxygenase (IDO1) inhibitors

In various embodiments, the FLT3L-Fc fusion proteins, homodimers, heterodimers, polynucleotides, vectors, lipoplexes, such as LNPs, and/or pharmaceutical compositions, as described herein, are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, and shIDO-ST, EOS-200271, KHK-2455, LY-3381916.

### Janus Kinase (JAK) Inhibitors

In various embodiments, the FLT3L-Fc fusion proteins, homodimers, heterodimers, polynucleotides, vectors, lipoplexes, such as LNPs, and/or pharmaceutical compositions, as described herein, are combined with an inhibitor of Janus kinase 1 (JAK1, JAK1A, JAK1B, JTK3; NCBI Gene ID: 3716); Janus kinase 2 (JAK2, JTK10, THCYT3; NCBI Gene ID: 3717); and/or Janus kinase 3 (JAK3, JAK-3, JAK3_HUMAN, JAKL, L-JAK, LJAK; NCBI Gene ID: 3718). Examples of JAK inhibitors include without limitation, AT9283, AZD1480, baricitinib, BMS-911543, fedratinib, filgotinib (GLPG0634), gandotinib (LY2784544), INCB039110 (itacitinib), lestaurtinib, momelotinib (CYT0387), NS-018, pacritinib (SB1518), peficitinib (ASP015K), ruxolitinib, tofacitinib (formerly tasocitinib), INCB052793, and XL019.

### Lysyl Oxidase-Like Protein (LOXL) Inhibitors

In various embodiments, the FLT3L-Fc fusion proteins, homodimers, heterodimers, polynucleotides, vectors, lipoplexes, such as LNPs, and/or pharmaceutical compositions, as described herein, are combined with an inhibitor of a LOXL protein, e.g., LOXL1 (NCBI Gene ID: 4016), LOXL2 (NCBI Gene ID: 4017), LOXL3 (NCBI Gene ID: 84695), LOXL4 (NCBI Gene ID: 84171), and/or LOX (NCBI Gene ID: 4015). Examples of LOXL inhibitors include without limitation, the antibodies described in WO 2009/017833 (Arresto Biosciences). Examples of LOXL2 inhibitors include without limitation, the antibodies described in WO 2009/017833 (Arresto Biosciences), WO 2009/035791 (Arresto Biosciences), and WO 2011/097513 (Gilead Biologics).

### Matrix Metalloprotease (MMP) Inhibitors

In various embodiments, the FLT3L-Fc fusion proteins, homodimers, heterodimers, polynucleotides, vectors, lipoplexes, such as LNPs, and/or pharmaceutical compositions, as described herein, are combined with an inhibitor of a matrix metallopeptidase (MMP), e.g., an inhibitor of MMP1 (NCBI Gene ID: 4312), MMP2 (NCBI Gene ID: 4313), MMP3 (NCBI Gene ID: 4314), MMP7 (NCBI Gene ID: 4316), MMP8 (NCBI Gene ID: 4317), MMP9 (NCBI Gene ID: 4318); MMP10 (NCBI Gene ID: 4319); MMP11 (NCBI Gene ID: 4320); MMP12 (NCBI Gene ID: 4321), MMP13 (NCBI Gene ID: 4322), MMP14 (NCBI Gene ID: 4323), MMP15 (NCBI Gene ID: 4324), MMP16 (NCBI Gene ID: 4325), MMP17 (NCBI Gene ID: 4326), MMP19 (NCBI Gene ID: 4327), MMP20 (NCBI Gene ID: 9313), MMP21 (NCBI Gene ID: 118856), MMP24 (NCBI Gene ID: 10893), MMP25 (NCBI Gene ID: 64386), MMP26 (NCBI Gene ID: 56547), MMP27 (NCBI Gene ID: 64066) and/or MMP28 (NCBI Gene ID: 79148). Examples of MMP9 inhibitors include without limitation, marimastat (BB-2516), cipemastat (Ro 32-3555), GS-5745 (andecaliximab) and those described in WO 2012/027721 (Gilead Biologics).

### RAS and RAS Pathway Inhibitors

In various embodiments, the FLT3L-Fc fusion proteins, homodimers, heterodimers, polynucleotides, vectors, lipoplexes, such as LNPs, and/or pharmaceutical compositions, as described herein, are combined with an inhibitor of KRAS proto-oncogene, GTPase (KRAS; a.k.a., NS; NS3; CFC2; RALD; K-Ras; KRAS1; KRAS2; RASK2; KI-RAS; C-K-RAS; K-RAS2A; K-RAS2B; K-RAS4A; K-RAS4B; c-Ki-ras2; NCBI Gene ID: 3845); NRAS proto-oncogene, GTPase (NRAS; a.k.a., NS6; CMNS; NCMS; ALPS4; N-ras; NRAS1; NCBI Gene ID: 4893); HRas proto-oncogene, GTPase (HRAS; a.k.a., CTLO; KRAS; HAMSV; HRAS1; KRAS2; RASH1; RASK2; Ki-Ras; p21ras; C—H-RAS; c-K-ras; H-RASIDX; c-Ki-ras; C-BAS/HAS; C-HA-RAS1; NCBI Gene ID: 3265). The Ras inhibitors can inhibit Ras at either the polynucleotide (e.g., transcriptional inhibitor) or polypeptide (e.g., GTPase enzyme inhibitor) level. In some embodiments, the inhibitors target one or more proteins in the Ras pathway, e.g., inhibit one or more of EGFR, Ras, Raf (A-Raf, B-Raf, C-Raf), MEK (MEK1, MEK2), ERK, PI3K, AKT and mTOR. Illustrative K-Ras inhibitors that can be co-administered include ARS-1620 (G12C), SML-8-73-1 (G12C), Compound 3144 (G12D), Kobe0065/2602 (Ras GTP), RT11, MRTX-849 (G12C) and K-Ras(G12D)-selective inhibitory peptides, including KRpep-2 (Ac-RRCPLYISYDPVCRR-NH₂) (SEQ ID NO:108) and KRpep-2d (Ac-RRRRCPLYISYDPVCRRRR-NH₂) (SEQ ID NO:109). Illustrative KRAS mRNA inhibitors include anti-KRAS U1 adaptor, AZD-4785, siG12D-LODER™, and siG12D exosomes. Illustrative MEK inhibitors that can be co-administered include binimetinib, cobimetinib, PD-0325901, pimasertib, RG-7304, selumetinib, trametinib, and those described below and herein. Illustrative Raf dimer inhibitors that can be co-administered BGB-283, HM-95573, LXH-254, LY-3009120, RG7304 and TAK-580. Illustrative ERK inhibitors that can be co-administered include LTT-462, LY-3214996, MK-8353, ravoxertinib and ulixertinib. Illustrative Ras GTPase inhibitors that can be co-administered include rigosertib. Illustrative PI3K inhibitors that can be co-administered include idelalisib (Zydelig®), alpelisib, buparlisib, pictilisib, and those described below and herein. Illustrative PI3K/mTOR inhibitors that can be co-administered include dactolisib, omipalisib and voxtalisib. In certain embodiments, Ras-driven cancers (e.g., NSCLC) having CDKN2A mutations can be inhibited by co-administration of the MEK inhibitor selumetinib and the CDK4/6 inhibitor palbociclib. See, e.g., Zhou, et al., Cancer Lett. 2017 Nov. 1; 408:130-137. Also, K-RAS and mutant N-RAS can be reduced by the irreversible ERBB1/2/4 inhibitor neratinib. See, e.g., Booth, et al., Cancer Biol Ther. 2018 Feb. 1; 19(2):132-137.

### Mitogen-Activated Protein Kinase (MEK) Inhibitors

In various embodiments, the FLT3L-Fc fusion proteins, homodimers, heterodimers, polynucleotides, vectors, lipoplexes, such as LNPs, and/or pharmaceutical compositions, as described herein, are combined with an inhibitor of mitogen-activated protein kinase kinase 7 (MAP2K7, JNKK2, MAPKK7, MEK, MEK 7, MKK7, PRKMK7, SAPKK-4, SAPKK4; NCBI Gene ID: 5609). Examples of MEK inhibitors include antroquinonol, binimetinib, cobimetinib (GDC-0973, XL-518), MT-144, selumetinib (AZD6244), sorafenib, trametinib (GSK1120212), uprosertib+trametinib, PD-0325901, pimasertib, LTT462, AS703988, CC-90003, refametinib.

### Phosphatidylinositol 3-Kinase (PI3K) Inhibitors

In various embodiments, the FLT3L-Fc fusion proteins, homodimers, heterodimers, polynucleotides, vectors, lipoplexes, such as an LNPs, and/or pharmaceutical compositions, as described herein, are combined with an inhibitor of a phosphatidylinositol-4,5-bisphosphate 3-kinase catalytic subunit, e.g., phosphatidylinositol-4,5-bisphosphate 3-kinase catalytic subunit alpha (PIK3CA, CLAPO, CLOVE, CWS5, MCAP, MCM, MCMTC, PI3K, PI3K-alpha, p110-alpha; NCBI Gene ID: 5290); phosphatidylinositol-4,5-bisphosphate 3-kinase catalytic subunit beta (PIK3CB, P110BETA, PI3K, PI3KBETA, PIK3C1; NCBI Gene ID: 5291); phosphatidylinositol-4,5-bisphosphate 3-kinase catalytic subunit gamma (PIK3CG, PI3CG, PI3K, PI3Kgamma, PIK3, p110gamma, p120-PI3K; Gene ID: 5494); and/or phosphatidylinositol-4,5-bisphosphate 3-kinase catalytic subunit delta (PIK3CD, APDS, IMD14, P110DELTA, PI3K, p110D, NCBI Gene ID: 5293). In some embodiments, the PI3K inhibitor is a pan-PI3K inhibitor. Examples of PI3K inhibitors include without limitation, ACP-319, AEZA-129, AMG-319, AS252424, AZD8186, BAY 10824391, BEZ235, buparlisib (BKM120), BYL719 (alpelisib), CH5132799, copanlisib (BAY 80-6946), duvelisib, GDC-0032, GDC-0077, GDC-0941, GDC-0980, GSK2636771, GSK2269557, idelalisib (Zydelig®), INCB50465, IPI-145, IPI-443, IPI-549, KAR4141, LY294002, LY3023414, MLN1117, OXY111A, PA799, PX-866, RG7604, rigosertib, RP5090, RP6530, SRX3177, taselisib, TG100115, TGR-1202 (umbralisib), TGX221, WX-037, X-339, X-414, XL147 (SAR245408), XL499, XL756, wortmannin, ZSTK474, and the compounds described in WO 2005/113556 (ICOS), WO 2013/052699 (Gilead Calistoga), WO 2013/116562 (Gilead Calistoga), WO 2014/100765 (Gilead Calistoga), WO 2014/100767 (Gilead Calistoga), and WO 2014/201409 (Gilead Sciences).

### Spleen Tyrosine Kinase (SYK) Inhibitors

In various embodiments, the FLT3L-Fc fusion proteins, homodimers, heterodimers, polynucleotides, vectors, lipoplexes, such as LNPs, and/or pharmaceutical compositions, as described herein, are combined with an inhibitor of spleen associated tyrosine kinase (SYK, p72-Syk, Gene ID: 6850). Examples of SYK inhibitors include without limitation, 6-(1H-indazol-6-yl)-N-(4-morpholinophenyl)imidazo[1,2-a]pyrazin-8-amine, BAY-61-3606, cerdulatinib (PRT-062607), entospletinib, fostamatinib (R788), HMPL-523, NVP-QAB 205 AA, R112, R343, tamatinib (R406), and those described in U.S. Pat. No. 8,450,321 (Gilead Conn.) and those described in U.S. 2015/0175616.

### Toll-Like Receptor (TLR) Agonists

In various embodiments, the FLT3L-Fc fusion proteins, homodimers, heterodimers, polynucleotides, vectors, lipoplexes, such as LNPs, and/or pharmaceutical compositions, as described herein, are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation DS-0509, GS-9620 (vesatolimod), vesatolimod analogs, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). An TLR7/TLR8 agonist that can be co-administered is NKTR-262. Example TLR8 agonists that can be co-administered include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Example TLR9 agonists that can be co-administered include without limitation AST-008, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, leftolimod (MGN-1703), CYT-003, CYT-003-QbG10 and PUL-042. Examples of TLR3 agonist include rintatolimod, poly-ICLC, RIBOXXON®, Apoxxim, RIBOXXIM®, IPH-33, MCT-465, MCT-475, and ND-1.1.

### Tyrosine-kinase Inhibitors (TKIs)

In various embodiments, the FLT3L-Fc fusion proteins, homodimers, heterodimers, polynucleotides, vectors, lipoplexes, such as LNPs, and/or pharmaceutical compositions, as described herein, are combined with a tyrosine kinase inhibitor (TKI). TKIs may target epidermal growth factor receptors (EGFRs) and receptors for fibroblast growth factor (FGF), platelet-derived growth factor (PDGF), and vascular endothelial growth factor (VEGF). Examples of TKIs include without limitation, afatinib, ARQ-087 (derazantinib), asp5878, AZD3759, AZD4547, bosutinib, brigatinib, cabozantinib, cediranib, crenolanib, dacomitinib, dasatinib, dovitinib, E-6201, erdafitinib, erlotinib, gefitinib, gilteritinib (ASP-2215), FP-1039, HM61713, icotinib, imatinib, KX2-391 (Src), lapatinib, lestaurtinib, lenvatinib, midostaurin, nintedanib, ODM-203, osimertinib (AZD-9291), ponatinib, poziotinib, quizartinib, radotinib, rociletinib, sulfatinib (HMPL-012), sunitinib, famitinib L-malate, (MAC-4), tivoanib, TH-4000, and MEDI-575 (anti-PDGFR antibody).

### Chemotherapeutic Agents

In various embodiments, the FLT3L-Fc fusion proteins, homodimers, heterodimers, polynucleotides, vectors, lipoplexes, such as LNPs, and/or pharmaceutical compositions, as described herein, are combined with a chemotherapeutic agent or anti-neoplastic agent.

As used herein, the term “chemotherapeutic agent” or “chemotherapeutic” (or “chemotherapy” in the case of treatment with a chemotherapeutic agent) is meant to encompass any non-proteinaceous (e.g., non-peptidic) chemical compound useful in the treatment of cancer. Examples of chemotherapeutic agents include but not limited to: alkylating agents such as thiotepa and cyclophosphamide (CYTOXAN®); alkyl sulfonates such as busulfan, improsulfan, and piposulfan; aziridines such as benzodepa, carboquone, meturedepa, and uredepa; ethylenimines and methylamelamines including altretamine, triethylenemelamine, triethylenephosphoramide, triethylenethiophosphoramide, and trimemylolomelamine; acetogenins, e.g., bullatacin and bullatacinone; a camptothecin, including synthetic analog topotecan; bryostatin, callystatin; CC-1065, including its adozelesin, carzelesin, and bizelesin synthetic analogs; cryptophycins, particularly cryptophycin 1 and cryptophycin 8; dolastatin; duocarmycin, including the synthetic analogs KW-2189 and CBI-TMI; eleutherobin; 5-azacytidine; pancratistatin; a sarcodictyin; spongistatin; nitrogen mustards such as chlorambucil, chlornaphazine, cyclophosphamide, glufosfamide, evofosfamide, bendamustine, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, foremustine, lomustine, nimustine, and ranimustine; antibiotics such as the enediyne antibiotics (e.g., calicheamicin, especially calicheamicin gammall and calicheamicin phill), dynemicin including dynemicin A, bisphosphonates such as clodronate, an esperamicin, neocarzinostatin chromophore and related chromoprotein enediyne antibiotic chromomophores, aclacinomycins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogs such as demopterin, methotrexate, pteropterin, and trimetrexate; purine analogs such as cladribine, pentostatin, fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replinishers such as frolinic acid; radiotherapeutic agents such as Radium-223; trichothecenes, especially T-2 toxin, verracurin A, roridin A, and anguidine; taxoids such as paclitaxel (TAXOL®), abraxane, docetaxel (TAXOTERE®), cabazitaxel, BIND-014, tesetaxel; platinum analogs such as cisplatin and carboplatin, NC-6004 nanoplatin; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; trabectedin, triaziquone; 2,2′,2″-trichlorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine (GEMZAR®); 6-thioguanine; mercaptopurine; methotrexate; vinblastine; platinum; etoposide (VP-16); ifosfamide; mitroxantrone; vancristine; vinorelbine (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; NUC-1031; FOLFOX (folinic acid, 5-fluorouracil, oxaliplatin); FOLFIRI (folinic acid, 5-fluorouracil, irinotecan); FOLFOXIRI (folinic acid, 5-fluorouracil, oxaliplatin, irinotecan), FOLFIRINOX (folinic acid, 5-fluorouracil, irinotecan, oxaliplatin), and pharmaceutically acceptable salts, acids, or derivatives of any of the above. Such agents can be conjugated onto an antibody or any targeting agent described herein to create an antibody-drug conjugate (ADC) or targeted drug conjugate.

### Anti-Hormonal Agents

Also included in the definition of “chemotherapeutic agent” are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors.

Examples of anti-estrogens and SERMs include, for example, tamoxifen (including NOLVADEX™), raloxifene, droloxifene, 4-hydroxytamoxifen, trioxifene, keoxifene, LY117018, onapristone, and toremifene (FARESTON®).

Inhibitors of the enzyme aromatase regulate estrogen production in the adrenal glands. Examples include 4(5)-imidazoles, aminoglutethimide, megestrol acetate (MEGACE®), exemestane, formestane, fadrozole, vorozole (RIVISOR®), letrozole (FEMARA®), and anastrozole (ARIMIDEX®).

Examples of anti-androgens include apalutamide, abiraterone, enzalutamide, flutamide, galeterone, nilutamide, bicalutamide, leuprolide, goserelin, ODM-201, APC-100, ODM-204.

An example progesterone receptor antagonist includes onapristone.

### Anti-Angiogenic Agents

In various embodiments, the FLT3L-Fc fusion proteins, homodimers, heterodimers, polynucleotides, vectors, lipoplexes, such as LNPs, and/or pharmaceutical compositions, as described herein, are combined with an anti-angiogenic agent. Anti-angiogenic agents that can be co-administered include, but are not limited to, retinoid acid and derivatives thereof, 2-methoxyestradiol, ANGIOSTATIN®, ENDOSTATIN®, regorafenib, necuparanib, suramin, squalamine, tissue inhibitor of metalloproteinase-1, tissue inhibitor of metalloproteinase-2, plasminogen activator inhibitor-1, plasminogen activator inbibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism including proline analogs such as 1-azetidine-2-carboxylic acid (LACA), cishydroxyproline, d,I-3,4-dehydroproline, thiaproline, α,α′-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chicken inhibitor of metalloproteinase-3 (ChIMP-3), chymostatin, beta-cyclodextrin tetradecasulfate, eponemycin, fumagillin, gold sodium thiomalate, d-penicillamine, beta-1-anticollagenase-serum, alpha-2-antiplasmin, bisantrene, lobenzarit disodium, n-2-carb oxyphenyl-4-chloroanthronilic acid disodium or “CCA”, thalidomide, angiostatic steroid, carboxy aminoimidazole, metalloproteinase inhibitors such as BB-94, inhibitors of S100A9 such as tasquinimod. Other anti-angiogenesis agents include antibodies, preferably monoclonal antibodies against these angiogenic growth factors: beta-FGF, alpha-FGF, FGF-5, VEGF isoforms, VEGF-C, HGF/SF, and Ang-1/Ang-2.

### Anti-Fibrotic Agents

In various embodiments, the FLT3L-Fc fusion proteins, homodimers, heterodimers, polynucleotides, vectors, lipoplexes, such as LNPs, and/or pharmaceutical compositions, as described herein, are combined with an anti-fibrotic agent. Anti-fibrotic agents that can be co-administered include, but are not limited to, the compounds such as beta-aminoproprionitrile (BAPN), as well as the compounds disclosed in U.S. Pat. No. 4,965,288 relating to inhibitors of lysyl oxidase and their use in the treatment of diseases and conditions associated with the abnormal deposition of collagen and U.S. Pat. No. 4,997,854 relating to compounds which inhibit LOX for the treatment of various pathological fibrotic states, which are herein incorporated by reference. Further exemplary inhibitors are described in U.S. Pat. No. 4,943,593 relating to compounds such as 2-isobutyl-3-fluoro-, chloro-, or bromo-allylamine, U.S. Pat. Nos. 5,021,456, 5,059,714, 5,120,764, 5,182,297, 5,252,608 relating to 2-(1-naphthyloxymemyl)-3-fluoroallylamine, and US 2004-0248871, which are herein incorporated by reference.

Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives; semicarbazide and urea derivatives; aminonitriles such as BAPN or 2-nitroethylamine; unsaturated or saturated haloamines such as 2-bromo-ethylamine, 2-chloroethylamine, 2-trifluoroethylamine, 3-bromopropylamine, and p-halobenzylamines; and selenohomocysteine lactone.

Other anti-fibrotic agents are copper chelating agents penetrating or not penetrating the cells. Exemplary compounds include indirect inhibitors which block the aldehyde derivatives originating from the oxidative deamination of the lysyl and hydroxylysyl residues by the lysyl oxidases. Examples include the thiolamines, particularly D-penicillamine, and its analogs such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, and sodium-4-mercaptobutanesulphinate trihydrate.

### Anti-Inflammatory Agents

In various embodiments, the FLT3L-Fc fusion proteins, homodimers, heterodimers, polynucleotides, vectors, lipoplexes, such as LNPs, and/or pharmaceutical compositions, as described herein, are combined with an anti-inflammatory agent. Example anti-inflammatory agents include without limitation inhibitors of one or more of arginase (ARG1 (NCBI Gene ID: 383), ARG2 (NCBI Gene ID: 384)), carbonic anhydrase (CA1 (NCBI Gene ID: 759), CA2 (NCBI Gene ID: 760), CA3 (NCBI Gene ID: 761), CA4 (NCBI Gene ID: 762), CASA (NCBI Gene ID: 763), CA5B (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CAl 1 (NCBI Gene ID: 770), CA12 (NCBI Gene ID: 771), CA13 (NCBI Gene ID: 377677), CA14 (NCBI Gene ID: 23632)), prostaglandin-endoperoxide synthase 1 (PTGS1, COX-1; NCBI Gene ID: 5742), prostaglandin-endoperoxide synthase 2 (PTGS2, COX-2; NCBI Gene ID: 5743), secreted phospholipase A2, prostaglandin E synthase (PTGES, PGES; Gene ID: 9536), arachidonate 5-lipoxygenase (ALOXS, 5-LOX; NCBI Gene ID: 240), soluble epoxide hydrolase 2 (EPHX2, SEH; NCBI Gene ID: 2053) and/or mitogen-activated protein kinase kinase kinase 8 (MAP3K8, TPL2; NCBI Gene ID: 1326). In some embodiments, the inhibitor is a dual inhibitor, e.g., a dual inhibitor of COX-2/COX-1, COX-2/SEH, COX-2/CA, COX-2/5-LOX.

Examples of inhibitors of prostaglandin-endoperoxide synthase 1 (PTGS1, COX-1; NCBI Gene ID: 5742) that can be co-administered include without limitation mofezolac, GLY-230, and TRK-700.

Examples of inhibitors of prostaglandin-endoperoxide synthase 2 (PTGS2, COX-2; NCBI Gene ID: 5743) that can be co-administered include without limitation diclofenac, meloxicam, parecoxib, etoricoxib, AP-101, celecoxib, AXS-06, diclofenac potassium, DRGT-46, AAT-076, meisuoshuli, lumiracoxib, meloxicam, valdecoxib, zaltoprofen, nimesulide, Anitrazafen, Apricoxib, Cimicoxib, Deracoxib, Flumizole, Firocoxib, Mavacoxib, NS-398, Pamicogrel, Parecoxib, Robenacoxib, Rofecoxib, Rutecarpine, Tilmacoxib, and Zaltoprofen. Examples of dual COX1/COX2 inhibitors that can be co-administered include without limitation, HP-5000, lornoxicam, ketorolac tromethamine, bromfenac sodium, ATB-346, HP-5000. Examples of dual COX-2/carbonic anhydrase (CA) inhibitors that can be co-administered include without limitation polmacoxib and imrecoxib.

Examples of inhibitors of secreted phospholipase A2, prostaglandin E synthase (PTGES, PGES; Gene ID: 9536) that can be co-administered include without limitation LY3023703, GRC 27864, and compounds described in WO2015158204, WO2013024898, WO2006063466, WO2007059610, WO2007124589, WO2010100249, WO2010034796, WO2010034797, WO2012022793, WO2012076673, WO2012076672, WO2010034798, WO2010034799, WO2012022792, WO2009103778, WO2011048004, WO2012087771, WO2012161965, WO2013118071, WO2013072825, WO2014167444, WO2009138376, WO2011023812, WO2012110860, WO2013153535, WO2009130242, WO2009146696, WO2013186692, WO2015059618, WO2016069376, WO2016069374, WO2009117985, WO2009064250, WO2009064251, WO2009082347, WO2009117987, and WO2008071173. Metformin has further been found to repress the COX2/PGE2/STAT3 axis, and can be co-administered. See, e.g., Tong, et al., Cancer Lett. (2017) 389:23-32; and Liu, et al., Oncotarget. (2016) 7(19):28235-46.

Examples of inhibitors of carbonic anhydrase (e.g., one or more of CA1 (NCBI Gene ID: 759), CA2 (NCBI Gene ID: 760), CA3 (NCBI Gene ID: 761), CA4 (NCBI Gene ID: 762), CASA (NCBI Gene ID: 763), CASB (NCBI Gene ID: 11238), CA6 (NCBI Gene ID: 765), CA7 (NCBI Gene ID: 766), CA8 (NCBI Gene ID: 767), CA9 (NCBI Gene ID: 768), CA10 (NCBI Gene ID: 56934), CAl 1 (NCBI Gene ID: 770), CA12 (NCBI Gene ID: 771), CA13 (NCBI Gene ID: 377677), CA14 (NCBI Gene ID: 23632)) that can be co-administered include without limitation acetazolamide, methazolamide, dorzolamide, zonisamide, brinzolamide and dichlorphenamide. A dual COX-2/CA1/CA2 inhibitor that can be co-administered includes CG100649.

Examples of inhibitors of arachidonate 5-lipoxygenase (ALOX5, 5-LOX; NCBI Gene ID: 240) that can be co-administered include without limitation meclofenamate sodium, zileuton.

Examples of inhibitors of soluble epoxide hydrolase 2 (EPHX2, SEH; NCBI Gene ID: 2053) that can be co-administered include without limitation compounds described in WO2015148954. Dual inhibitors of COX-2/SEH that can be co-administered include compounds described in WO2012082647. Dual inhibitors of SEH and fatty acid amide hydrolase (FAAH; NCBI Gene ID: 2166) that can be co-administered include compounds described in WO2017160861.

Examples of inhibitors of mitogen-activated protein kinase kinase kinase 8 (MAP3K8, tumor progression loci-2, TPL2; NCBI Gene ID: 1326) that can be co-administered include without limitation GS-4875, GS-5290, BHM-078 and those described, e.g., in WO2006124944, WO2006124692, WO2014064215, WO2018005435, Teli, et al., J Enzyme Inhib Med Chem. (2012) 27(4):558-70; Gangwall, et al., Curr Top Med Chem. (2013) 13(9):1015-35; Wu, et al., Bioorg Med Chem Lett. (2009) 19(13):3485-8; Kaila, et al., Bioorg Med Chem. (2007) 15(19):6425-42; and Hu, et al., Bioorg Med Chem Lett. (2011) 21(16):4758-61.

### Tumor Oxygenation Agents

In various embodiments, the FLT3L-Fc fusion proteins, homodimers, heterodimers, polynucleotides, vectors, lipoplexes, such as LNPs, and/or pharmaceutical compositions, as described herein, are combined with an agent that promotes or increases tumor oxygenation or reoxygenation, or prevents or reduces tumor hypoxia. Illustrative agents that can be co-administered include, e.g., Hypoxia inducible factor-1 alpha (HIF-1α) inhibitors, such as PT-2977, PT-2385; VEGF inhibitors, such as bevasizumab, IMC-3C5, GNR-011, tanibirumab, LYN-00101, ABT-165; and/or an oxygen carrier protein (e.g., a heme nitric oxide and/or oxygen binding protein (HNOX)), such as OMX-302 and HNOX proteins described in WO 2007/137767, WO 2007/139791, WO 2014/107171, and WO 2016/149562.

### Immunotherapeutic Agents

In various embodiments, the FLT3L-Fc fusion proteins, homodimers, heterodimers, polynucleotides, vectors, lipoplexes, such as LNPs, and/or pharmaceutical compositions, as described herein, are combined with an immunotherapeutic agent. Example immunotherapeutic agents that can be co-administered include without limitation abagovomab, ABP-980, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, CC49, cetuximab, citatuzumab, cixutumumab, clivatuzumab, conatumumab, dacetuzumab, dalotuzumab, daratumumab, detumomab, dinutuximab, drozitumab, duligotumab, dusigitumab, ecromeximab, elotuzumab, emibetuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab (YERVOY®, MDX-010, BMS-734016, and MDX-101), iratumumab, labetuzumab, lexatumumab, lintuzumab, lorvotuzumab, lucatumumab, mapatumumab, matuzumab, milatuzumab, minretumomab, mitumomab, mogamulizumab, moxetumomab, naptumomab, narnatumab, necitumumab, nimotuzumab, nofetumomab, OBI-833, obinutuzumab, ocaratuzumab, ofatumumab, olaratumab, onartuzumab, oportuzumab, oregovomab, panitumumab, parsatuzumab, pasudotox, patritumab, pemtumomab, pertuzumab, pintumomab, pritumumab, racotumomab, radretumab, ramucirumab (Cyramza®), rilotumumab, rituximab, robatumumab, samalizumab, satumomab, sibrotuzumab, siltuximab, solitomab, simtuzumab, tacatuzumab, taplitumomab, tenatumomab, teprotumumab, tigatuzumab, tositumomab, trastuzumab, tucotuzumab, ubilituximab, veltuzumab, vorsetuzumab, votumumab, zalutumumab, and 3F8. Rituximab can be used for treating indolent B-cell cancers, including marginal-zone lymphoma, WM, CLL and small lymphocytic lymphoma. A combination of Rituximab and chemotherapy agents is especially effective.

The exemplified therapeutic antibodies may be further labeled or combined with a radioisotope particle such as indium-111, yttrium-90 (90Y-clivatuzumab), or iodine-131.

In some embodiments, the immunotherapeutic agent is an antibody-drug conjugate (ADC). Illustrative ADCs that can be co-administered include without limitation drug-conjugated antibodies, fragments thereof, or antibody mimetics targeting the proteins or antigens listed above and herein (e.g., in Table B). Example ADCs that can be co-administered include without limitation gemtuzumab, brentuximab, trastuzumab, inotuzumab, glembatumumab, anetumab, mirvetuximab, depatuxizumab, rovalpituzumab, vadastuximab, labetuzumab, sacituzumab, lifastuzumab, indusatumab, polatzumab, pinatuzumab, coltuximab, indatuximab, milatuzumab, rovalpituzumab, ABBV-399, AGS-16C3F, ASG-22ME, AGS67E, AMG172, AMG575, BAY1129980, BAY1187982, BAY94-9343, GSK2857916, Humax-TF-ADC, IMGN289, IMGN529, IMGN853, LOP628, PCA062, MDX-1203 (BMS936561), MEDI-547, PF-06263507, PF-06647020, PF-06647263, PF-06664178, RG7450, RG7458, RG7598, SAR566658, SGN-CD19A, SGN-CD33A, SGN-CD70A, SGN-LIV1A and SYD985. ADCs that can be co-administered are described, e.g., in Lambert, et al., Adv Ther (2017) 34:1015-1035 and in de Goeij, Current Opinion in Immunology (2016) 40:14-23.

Illustrative therapeutic agents (e.g., anticancer or antineoplastic agents) that can be conjugated to the drug-conjugated antibodies, fragments thereof, or antibody mimetics include without limitation monomethyl auristatin E (MMAE), monomethyl auristatin F (MMAF), a calicheamicin, ansamitocin, maytansine or an analog thereof (e.g., mertansine/emtansine (DM1), ravtansine/soravtansine (DM4)), an anthracyline (e.g., doxorubicin, daunorubicin, epirubicin, idarubicin), pyrrolobenzodiazepine (PBD) DNA cross-linking agent SC-DR002 (D6.5), duocarmycin, a microtubule inhibitors (MTI) (e.g., a taxane, a vinca alkaloid, an epothilone), a pyrrolobenzodiazepine (PBD) or dimer thereof, a duocarmycin (A, B1, B2, C1, C2, D, SA, CC-1065), and other anticancer or anti-neoplastic agents described herein. In some embodiments, the therapeutic agents (e.g., anticancer or antineoplastic agents) that can be conjugated to the drug-conjugated antibodies, fragments thereof, or antibody mimetics include an immune checkpoint inhibitor. In some embodiments, the conjugated immune checkpoint inhibitor is a conjugated small molecule inhibitor of CD274 (PDL1, PD-L1), programmed cell death 1 (PDCD1, PD1, PD-1) or CTLA4. In some embodiments, the conjugated small molecule inhibitor of CD274 or PDCD1 is selected from the group consisting of GS-4224, GS-4416, INCB086550 and MAX10181. In some embodiments, the conjugated small molecule inhibitor of CTLA4 comprises BPI-002.

### Cancer Gene Therapy and Cell Therapy

In various embodiments, the FLT3L-Fc fusion proteins, homodimers, heterodimers, polynucleotides, vectors, lipoplexes, such as LNPs, and/or pharmaceutical compositions, as described herein, are combined with a cancer gene therapy and cell therapy. Cancer gene therapies and cell therapies include the insertion of a normal gene into cancer cells to replace a mutated or altered gene; genetic modification to silence a mutated gene; genetic approaches to directly kill the cancer cells; including the infusion of immune cells designed to replace most of the patient's own immune system to enhance the immune response to cancer cells, or activate the patient's own immune system (T cells or Natural Killer cells) to kill cancer cells, or find and kill the cancer cells; genetic approaches to modify cellular activity to further alter endogenous immune responsiveness against cancer.

### Cellular Therapies

In various embodiments, the FLT3L-Fc fusion proteins, homodimers, heterodimers, polynucleotides, vectors, lipoplexes, such as LNPs, and/or pharmaceutical compositions, as described herein, are combined with one or more cellular therapies. Illustrative cellular therapies include without limitation co-administration of one or more of a population of natural killer (NK) cells, NK-T cells, T cells, cytokine-induced killer (CIK) cells, macrophage (MAC) cells, tumor infiltrating lymphocytes (TILs) and/or dendritic cells (DCs). In some embodiments, the cellular therapy entails a T cell therapy, e.g., co-administering a population of alpha/beta TCR T cells, gamma/delta TCR T cells, regulatory T (Treg) cells and/or TRuC™ T cells. In some embodiments, the cellular therapy entails a NK cell therapy, e.g., co-administering NK-92 cells. As appropriate, a cellular therapy can entail the co-administration of cells that are autologous, syngeneic or allogeneic to the subject.

In some embodiments, the cellular therapy entails co-administering cells comprising chimeric antigen receptors (CARs). In such therapies, a population of immune effector cells engineered to express a CAR, wherein the CAR comprises a tumor antigen-binding domain. In T cell therapies, the T cell receptors (TCRs) are engineered to target tumor derived peptides presented on the surface of tumor cells.

With respect to the structure of a CAR, in some embodiments, the CAR comprises an antigen binding domain, a transmembrane domain, and an intracellular signaling domain. In some embodiments, the intracellular domain comprises a primary signaling domain, a costimulatory domain, or both of a primary signaling domain and a costimulatory domain. In some embodiments, the primary signaling domain comprises a functional signaling domain of one or more proteins selected from the group consisting of CD3 zeta, CD3 gamma, CD3 delta, CD3 epsilon, common FcR gamma (FCERIG), FcR beta (Fc Epsilon Rlb), CD79a, CD79b, Fcgamma RIIa, DAP10, and DAP12.

In some embodiments, the costimulatory domain comprises a functional domain of one or more proteins selected from the group consisting of CD27, CD28, 4-1BB(CD137), OX40, CD30, CD40, PD-1, ICOS, CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRFI), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, ITGAE, CD103, ITGAL, CD1A (NCBI Gene ID: 909), CD1B (NCBI Gene ID: 910), CD1C (NCBI Gene ID: 911), CD1D (NCBI Gene ID: 912), CD1E (NCBI Gene ID: 913), ITGAM, ITGAX, ITGB1, CD29, ITGB2 (CD18, LFA-1), ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

In some embodiments, the transmembrane domain comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, ICOS (CD278), 4-1BB(CD137), GITR, CD40, BAFFR, HVEM (LIGHTR), SLAMF7, NKp80 (KLRF1), CD160, CD19, IL2R beta, IL2R gamma, IL7R, ITGA1, VLA1, CD49a, ITGA4, IA4, CD49D, ITGA6, VLA-6, CD49f, ITGAD, CD1A, CD1B, CD1C, CD1D, CD1E, ITGAE, CD103, ITGAL, ITGAM, ITGAX, ITGB1, CD29, ITGB2 (LFA-1, CD18), ITGB7, TNFR2, DNAM1 (CD226), SLAMF4 (CD244, 2B4), CD84, CD96 (TACTILE), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C.

In some embodiments, the TCR or CAR antigen binding domain or the immunotherapeutic agent described herein (e.g., monospecific or multi-specific antibody or antigen-binding fragment thereof or antibody mimetic) binds a tumor-associated antigen (TAA). In some embodiments, the tumor-associated antigen is selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECLI); CD33; epidermal growth factor receptor variant III (EGFRvlll); ganglioside G2 (GD2); ganglioside GD3 (αNeuSAc(2-8)αNeuSAc(2-3)βDGaip(1-4)bDGIcp(1-1)Cer); ganglioside GM3 (αNeuSAc(2-3)βDGalp(1-4)(3DGlcp(1-1)Cer); TNF receptor superfamily member 17 (TNFRSF17, BCMA); Tn antigen ((Tn Ag) or (GalNAcu-Ser/Thr)); prostate-specific membrane antigen (PSMA); Receptor tyrosine kinase-like orphan receptor 1 (RORI); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y)antigen; CD24; Platelet-derived growth factor receptor beta (PDGFR-beta); Stage-specific embryonic antigen-4 (SSEA-4); CD20; delta like 3 (DLL3); Folate receptor alpha; Receptor tyrosine-protein kinase, ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2(EphA2); Fucosyl GM1; sialyl Lewis adhesion molecule (sLe); transglutaminase 5 (TGS5); high molecular weight-melanoma associated antigen (HMWMAA); o-acetyl-GD2 ganglioside (OAcGD2); Folate receptor beta; tumor endothelial marker 1 (TEM1/CD248); tumor endothelial marker 7-related (TEM7R); six transmembrane epithelial antigen of the prostate I (STEAP1); claudin 6 (CLDN6); thyroid stimulating hormone receptor (TSHR); G protein-coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (ORS IE2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MADCT-1); melanoma cancer testis antigen-2 (MAD-CT-2); Fos-related antigen 1; tumor protein p53, (p53); p53 mutant; prostein; survivin; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP IBI); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES I); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-I); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRI); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1). In some embodiments, the target is an epitope of the tumor associated antigen presented in an MHC.

In some embodiments, the tumor antigen is selected from CD150, 5T4, ActRIIA, B7, TNF receptor superfamily member 17 (TNFRSF17, BCMA), CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-1R, IL-11Ralpha, IL-13R-alpha2, IL-2, IL-22R-alpha, IL-6, IL-6R, Ia, L1-CAM, L1-cell adhesion molecule, Lewis Y, L1-CAM, MAGE A3, MAGE-A1, MART-1, MUC1, NKG2C ligands, NKG2D Ligands, NYESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-I, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D1), ephrinB2, epithelial tumor antigen, estrogen receptor, fetal acetylcholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, P2-Microgiobuiin, Fc Receptor-like 5 (FcRL5).

In some embodiments, the antigen binding domain binds to an epitope of a target or tumor associated antigen (TAA) presented in a major histocompatibility complex (MHC) molecule. In some embodiments, the TAA is a cancer testis antigen. In some embodiments, the cancer testis antigen is selected from the group consisting of acrosin binding protein (ACRBP; CT23, OY-TES-1, SP32; NCBI Gene ID: 84519), alpha fetoprotein (AFP; AFPD, FETA, HPAFP; NCBI Gene ID: 174); A-kinase anchoring protein 4 (AKAP4; AKAP 82, AKAP-4, AKAP82, CT99, FSC1, HI, PRKA4, hAKAP82, p82; NCBI Gene ID: 8852), ATPase family AAA domain containing 2 (ATAD2; ANCCA, CT137, PRO2000; NCBI Gene ID: 29028), kinetochore scaffold 1 (KNL1; AF15Q14, CASC5, CT29, D40, MCPH4, PPP1R55, Spc7, hKNL-1, hSpc105; NCBI Gene ID: 57082), centrosomal protein 55 (CEP55; C10orf3, CT111, MARCH, URCC6; NCBI Gene ID: 55165), cancer/testis antigen 1A (CTAG1A; ESO1; CT6.1; LAGE-2; LAGE2A; NY-ESO-1; NCBI Gene ID: 246100), cancer/testis antigen 1B (CTAG1B; CT6.1, CTAG, CTAG1, ESO1, LAGE-2, LAGE2B, NY-ESO-1; NCBI Gene ID: 1485), cancer/testis antigen 2 (CTAG2; CAMEL, CT2, CT6.2, CT6.2a, CT6.2b, ESO2, LAGE-1, LAGE2B; NCBI Gene ID: 30848), CCCTC-binding factor like (CTCFL; BORIS, CT27, CTCF-T, HMGB1L1, dJ579F20.2; NCBI Gene ID: 140690), catenin alpha 2 (CTNNA2; CAP-R, CAPR, CDCBM9, CT114, CTNR; NCBI Gene ID: 1496), cancer/testis antigen 83 (CT83; CXorf61, KK-LC-1, KKLC1; NCBI Gene ID: 203413), cyclin A1 (CCNA1; CT146; NCBI Gene ID: 8900), DEAD-box helicase 43 (DDX43; CT13, HAGE; NCBI Gene ID: 55510), developmental pluripotency associated 2 (DPPA2; CT100, ECAT15-2, PESCRG1; NCBI Gene ID: 151871), fetal and adult testis expressed 1 (FATE1; CT43, FATE; NCBI Gene ID: 89885), FMR1 neighbor (FMR1NB; CT37, NY-SAR-35, NYSAR35; NCBI Gene ID: 158521), HORMA domain containing 1 (HORMAD1; CT46, NORMA; NCBI Gene ID: 84072), insulin like growth factor 2 mRNA binding protein 3 (IGF2BP3; CT98, IMP-3, IMP3, KOC, KOC1, VICKZ3; NCBI Gene ID: 10643), leucine zipper protein 4 (LUZP4; CT-28, CT-8, CT28, HOM-TES-85; NCBI Gene ID: 51213), lymphocyte antigen 6 family member K (LY6K; CT97, HSJ001348, URLC10, ly-6K; NCBI Gene ID: 54742), maelstrom spermatogenic transposon silencer (MAEL; CT128, SPATA35; NCBI Gene ID: 84944), MAGE family member A1 (MAGEA1; CT1.1, MAGE1; NCBI Gene ID: 4100); MAGE family member A3 (MAGEA3; CT1.3, HIPS, HYPD, MAGE3, MAGEA6; NCBI Gene ID: 4102); MAGE family member A4 (MAGEA4; CT1.4, MAGE-41, MAGE-X2, MAGE4, MAGE4A, MAGE4B; NCBI Gene ID: 4103); MAGE family member A11 (MAGEA11; CT1.11, MAGE-11, MAGE11, MAGEA-11; NCBI Gene ID: 4110); MAGE family member C1 (MAGEC1; CT7, CT7.1; NCBI Gene ID: 9947); MAGE family member C2 (MAGEC2; CT10, HCA587, MAGEE1; NCBI Gene ID: 51438); MAGE family member D1 (MAGED1; DLXIN-1, NRAGE; NCBI Gene ID: 9500); MAGE family member D2 (MAGED2; 11B6, BARTSS, BCG-1, BCG1, HCA10, MAGE-D2; NCBI Gene ID: 10916), kinesin family member 20B (KIF20B; CT90, KRMP1, MPHOSPH1, MPP-1, MPP1; NCBI Gene ID: 9585), NUF2 component of NDC80 kinetochore complex (NUF2; CDCA1, CT106, NUF2R; NCBI Gene ID: 83540), nuclear RNA export factor 2 (NXF2; CT39, TAPL-2, TCP11X2; NCBI Gene ID: 56001), PAS domain containing repressor 1 (PASD1; CT63, CT64, OXTES1; NCBI Gene ID: 139135), PDZ binding kinase (PBK; CT84, HEL164, Nori-3, SPK, TOPK; NCBI Gene ID: 55872), piwi like RNA-mediated gene silencing 2 (PIWIL2; CT80, HILI, PIWIL1L, mili; NCBI Gene ID: 55124), preferentially expressed antigen in melanoma (PRAME; CT130, MAPE, OIP-4, OIP4; NCBI Gene ID: 23532), sperm associated antigen 9 (SPAG9; CT89, HLC-6, HLC4, HLC6, JIP-4, JIP4, JLP, PNET, PIG6; NCBI Gene ID: 9043), sperm protein associated with the nucleus, X-linked, family member A1 (SPANXA1; CT11.1, CT11.3, NAP-X, SPAN-X, SPAN-Xa, SPAN-Xb, SPANX, SPANX-A; NCBI Gene ID: 30014), SPANX family member A2 (SPANXA2; CT11.1, CT11.3, SPANX, SPANX-A, SPANX-C, SPANXA, SPANXC; NCBI Gene ID: 728712), SPANX family member C (SPANXC; CT11.3, CTp11, SPANX-C, SPANX-E, SPANXE; NCBI Gene ID: 64663), SPANX family member D (SPANXD; CT11.3, CT11.4, SPANX-C, SPANX-D, SPANX-E, SPANXC, SPANXE, dJ171K16.1; NCBI Gene ID: 64648), SSX family member 1 (SSX1; CT5.1, SSRC; NCBI Gene ID: 6756), SSX family member 2 (SSX2; CT5.2, CT5.2A, HD21, HOM-MEL-40, SSX; NCBI Gene ID: 6757), synaptonemal complex protein 3 (SYCP3; COR1, RPRGL4, SCP3, SPGF4; NCBI Gene ID: 50511), testis expressed 14, intercellular bridge forming factor (TEX14; CT113, SPGF23; NCBI Gene ID: 56155), transcription factor Dp family member 3 (TFDP3; CT30, DP4, HCA661; NCBI Gene ID: 51270), serine protease 50 (PRSS50; CT20, TSP50; NCBI Gene ID: 29122), TTK protein kinase (TTK; CT96, ESK, MPH1, MPS1, MPS1L1, PYT; NCBI Gene ID: 7272) and zinc finger protein 165 (ZNF165; CT53, LD65, ZSCAN7; NCBI Gene ID: 7718). T cell receptors (TCRs) and TCR-like antibodies that bind to an epitope of a cancer testis antigen presented in a major histocompatibility complex (MHC) molecule are known in the art and can be used in the herein described heterodimers. Cancer testis antigens associated with neoplasia are summarized, e.g., in Gibbs, et al., Trends Cancer 2018 October; 4(10):701-712 and the CT database website at cta.lncc.br/index.php. Illustrative TCRs and TCR-like antibodies that bind to an epitope of NY-ESO-1 presented in an MHC are described, e.g., in Stewart-Jones, et al., Proc Natl Acad Sci USA. 2009 Apr. 7; 106(14):5784-8; WO2005113595, WO2006031221, WO2010106431, WO2016177339, WO2016210365, WO2017044661, WO2017076308, WO2017109496, WO2018132739, WO2019084538, WO2019162043, WO2020086158 and WO2020086647. Illustrative TCRs and TCR-like antibodies that bind to an epitope of PRAME presented in an MHC are described, e.g., in WO2011062634, WO2016142783, WO2016191246, WO2018172533, WO2018234319 and WO2019109821. Illustrative TCRs and TCR-like antibodies that bind to an epitope of a MAGE variant presented in an MHC are described, e.g., in WO2007032255, WO2012054825, WO2013039889, WO2013041865, WO2014118236, WO2016055785, WO2017174822, WO2017174823, WO2017174824, WO2017175006, WO2018097951, WO2018170338, WO2018225732 and WO2019204683. Illustrative TCRs and TCR-like antibodies that bind to an epitope of alpha fetoprotein (AFP) presented in an MHC are described, e.g., in WO2015011450. Illustrative TCRs and TCR-like antibodies that bind to an epitope of SSX2 presented in an MHC are described, e.g., in WO2020063488. Illustrative TCRs and TCR-like antibodies that bind to an epitope of KK-LC-1 (CT83) presented in an MHC are described, e.g., in WO2017189254.

Examples of cell therapies include without limitation: Algenpantucel-L, Sipuleucel-T, (BPX-501) rivogenlecleucel U.S. Pat. No. 9,089,520, WO2016100236, AU-105, ACTR-087, activated allogeneic natural killer cells CNDO-109-AANK, MG-4101, AU-101, BPX-601, FATE-NK100, LFU-835 hematopoietic stem cells, Imilecleucel-T, baltaleucel-T, PNK-007, UCARTCS1, ET-1504, ET-1501, ET-1502, ET-190, CD19-ARTEMIS, ProHema, FT-1050-treated bone marrow stem cell therapy, CD4CARNK-92 cells, CryoStim, AlloStim, lentiviral transduced huCART-meso cells, CART-22 cells, EGFRt/19-28z/4-1BBL CAR T cells, autologous 4H11-28z/fIL-12/EFGRt T cell, CCR5-SBC-728-HSPC, CAR4-1BBZ, CH-296, dnTGFbRII-NY-ESOc259T, Ad-RTS-IL-12, IMA-101, IMA-201, CARMA-0508, TT-18, CMD-501, CMD-503, CMD-504, CMD-502, CMD-601, CMD-602, CSG-005.

Additional agents for targeting tumors include without limitation:
Alpha-fetoprotein, such as ET-1402, and AFP-TCR;
Anthrax toxin receptor 1, such as anti-TEM8 CAR T-cell therapy;
TNF receptor superfamily member 17 (TNFRSF17, BCMA), such as bb-2121, UCART-BCMA, ET-140, KITE-585, MCM-998, LCAR-B38M, CART-BCMA, SEA-BCMA, BB212, UCART-BCMA, ET-140, P-BCMA-101, AUTO-2 (APRIL-CAR);
Anti-CLL-1 antibodies, such as KITE-796;
Anti-PD-L1-CAR tank cell therapy, such as KD-045;
B7 homolog 6, such as CAR-NKp30 and CAR-B7H6;
B-lymphocyte antigen CD19, such as TBI-1501, CTL-119 huCART-19 T cells, JCAR-015 U.S. Pat. No. 7,446,190, JCAR-014, JCAR-017, (WO2016196388, WO2016033570, WO2015157386), axicabtagene ciloleucel (KTE-C19, Yescarta®), KTE-X19, U.S. Pat. Nos. 7,741,465, 6,319,494, UCART-19, EBV-CTL, T tisagenlecleucel-T (CTL019), WO2012079000, WO2017049166, CD19CAR-CD28-CD3zeta-EGFRt-expressing T cells, CD19/4-1BBL armored CAR T cell therapy, C-CAR-011, CIK-CAR.CD19, CD19CAR-28-zeta T cells, PCAR-019, MatchCART, DSCAR-01, IM19 CAR-T, TC-110;
B-lymphocyte antigen CD20, such as ACTR707 ATTCK-20;
B-lymphocyte antigen CD19/B-lymphocyte antigen 22, such as TC-310;
B-lymphocyte antigen 22 cell adhesion, such as UCART-22, JCAR-018 WO2016090190;
NY-ESO-1, such as GSK-3377794, TBI-1301;
Carbonic anhydrase, such as DC-Ad-GMCAIX;
Caspase 9 suicide gene, such as CaspaCIDe DLI, BPX-501;
CCR5, such as SB-728;
CDw123, such as MB-102, UCART-123;
CD4, such as ICG-122;
CD33, such as CIK-CAR.CD33;
CD38, such as T-007, UCART-38;
CD40 ligand, such as BPX-201;
CEACAM protein 5 modulators, such as MG7-CART;
Claudin 6, such as CSG-002;
EBV targeted, such as CMD-003;
MUC16EGFR, such as autologous 4H11-28z/fIL-12/EFGRt T cell;
Endonuclease, such as PGN-514, PGN-201;
Epstein-Barr virus specific T-lymphocytes, such as TT-10;
Erbb2, such as CST-102, CIDeCAR;
Ganglioside (GD2), such as 4SCAR-GD2;
folate hydrolase 1 (FOLH1, Glutamate carboxypeptidase II, PSMA; NCBI Gene ID: 2346), such as CIK-CAR.PSMA, CART-PSMA-TGFβRDN, P-PSMA-101;
Glypican-3 (GPC3), such as TT-16, GLYCAR;
Hemoglobin, such as PGN-236;
Hepatocyte growth factor receptor, such as anti-cMet RNA CAR T;
Human papillomavirus E7 protein, such as KITE-439;
Immunoglobulin gamma Fc receptor III, such as ACTR087;
IL-12, such as DC-RTS-IL-12;
IL-12 agonist/mucin 16, such as JCAR-020;
IL-13 alpha 2, such as MB-101;
IL-2, such as CST-101;
K-Ras GTPase, such as anti-KRAS G12V mTCR cell therapy;
Neural cell adhesion molecule L1 L1CAM (CD171), such as JCAR-023;
Latent membrane protein 1/Latent membrane protein 2, such as Ad5f35-LMPd1-2-transduced autologous dendritic cells;
Melanoma associated antigen 10, such as MAGE-A10C796T MAGE-A10 TCR;
Melanoma associated antigen 3/Melanoma associated antigen 6 (MAGE A3/A6) such as KITE-718;
Mesothelin, such as CSG-MESO, TC-210;
NKG2D, such as NKR-2;
Ntrkr1 tyrosine kinase receptor, such as JCAR-024;
PRAMET cell receptor, such as BPX-701;
T-lymphocyte, such as TT-12;
Tumor infiltrating lymphocytes, such as LN-144, LN-145; and/or
Wilms tumor protein, such as JTCR-016, WT1-CTL.

In various embodiments, the FLT3L-Fc fusion proteins, homodimers, heterodimers, polynucleotides, vectors, lipoplexes, such as LNPs, and/or pharmaceutical compositions, as described herein, are combined with a gene or cell therapy regimen that can target a cell infected with a virus (e.g., HIV). A gene or cell therapy that can be combined with an agent disclosed herein includes without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Illustrative examples of a cell therapy that can be combined with an agent disclosed herein include LB-1903, ENOB-HV-01, GOVX-B01, and SupT1 cell-based therapy. Illustrative examples of a dendritic cell therapy that can be combined with an agent disclosed herein include AGS-004. An illustrative example of a CCR5 gene editing agent that can be used in combination with an agent disclosed herein is SB-728T. An illustrative example of a CCR5 gene inhibitor that can be used in combination with an agent disclosed herein is Cal-1. In some embodiments, C34-CCR5/C34-CXCR4 expressing CD4-positive T-cells are co-administered with an agent disclosed herein. In some embodiments, the agents described herein are co-administered with AGT-103-transduced autologous T-cell therapy or AAV-eCD4-Ig gene therapy.

In various embodiments, the FLT3L-Fc fusion proteins, homodimers, heterodimers, polynucleotides, vectors, lipoplexes, such as LNPs, and/or pharmaceutical compositions, as described herein, can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include convertible CAR-T, VC-CAR-T, CMV-N6-CART, anti-CD4 CART-cell therapy, CD4 CAR+C34-CXCR4+CCR5 ZFN T-cells, autologous hematopoietic stem cells genetically engineered to express a CD4 CAR and the C46 peptide.

In various embodiments, the FLT3L-Fc fusion proteins, homodimers, heterodimers, polynucleotides, vectors, lipoplexes, such as LNPs, and/or pharmaceutical compositions, as described herein, are combined with a population of B cells genetically modified to express broadly neutralizing antibodies, such as 3BNC117 (Hartweger et al., J. Exp. Med. 2019, 1301; Moffett et al., Sci. Immunol. 4, eaax0644 (2019) 17 May 2019).

### Gene Editors

In various embodiments, the FLT3L-Fc fusion proteins, homodimers, heterodimers, polynucleotides, vectors, lipoplexes, such as LNPs, and/or pharmaceutical compositions, as described herein, are combined with gene editor. Illustrative gene editing system that can be co-administered include without limitation a CRISPR/Cas9 system, a zinc finger nuclease system, a TALEN system, a homing endonucleases system (e.g., an ARCUS), and a homing meganuclease system.

### CDK Inhibitors

In various embodiments, the FLT3L-Fc fusion proteins, homodimers, heterodimers, polynucleotides, vectors, lipoplexes, such as LNPs, and/or pharmaceutical compositions, as described herein, are combined with a CDK inhibitor such as VS2-370.

### STING Agonists, RIG-I and NOD2 Modulators

In various embodiments, the FLT3L-Fc fusion proteins, homodimers, heterodimers, polynucleotides, vectors, lipoplexes, such as LNPs, and/or pharmaceutical compositions, as described herein, are combined with a stimulator of interferon genes (STING) agonist or activator, a RIG-I modulator (e.g., RGT-100), or a NOD2 modulator (e.g., SB-9200, IR-103). In some embodiments, the STING receptor agonist or activator that can be co-administered with an agent of this disclosure is selected from ADU-S100 (MIW-815), SB-11285, MK-1454, SR-8291, AdVCA0848, GSK-532, SYN-STING, MSA-1, SR-8291, 5,6-dimethylxanthenone-4-acetic acid (DMXAA), cyclic-GAMP (cGAMP) and cyclic-di-AMP. In some embodiments, the STING agonist is selected from the compounds disclosed in WO 2018065360 (“Biolog Life Science Institute Forschungslabor and Biochemica-Vertrieb GmbH, Germany), WO 2018009466 (Aduro Biotech), WO 2017186711 (InvivoGen), WO 2017161349 (Immune Sensor), WO 2017106740 (Aduro Biotech), US 20170158724 (Glaxo Smithkiline), WO 2017075477 (Aduro Biotech), US 20170044206 (Merck), WO 2014179760 (University of California), WO2018098203 (Janssn), WO2018118665 (Merck), WO2018118664 (Merck), WO2018100558 (Takeda), WO2018067423 (Merck), and WO2018060323 (Boehringer).

### LAG-3 and TIM-3 Inhibitors

In various embodiments, the FLT3L-Fc fusion proteins, homodimers, heterodimers, polynucleotides, vectors, lipoplexes, such as LNPs, and/or pharmaceutical compositions, as described herein, are combined with a LAG-3 inhibitor or a TIM-3 inhibitor. In some embodiments, the LAG-3 inhibitor that can be co-administered with an agent of this disclosure is selected from relatlimab (ONO-4482), LAG-525, MK-4280, REGN-3767, and INCAGN2385. In some embodiments, the TIM-3 inhibitor that can be co-administered with an agent of this disclosure is an anti-TIM-3 antibody, such as TSR-022, LY-3321367, MBG-453, or INCAGN-2390.

### Interleukine Agonists

In various embodiments, the FLT3L-Fc fusion proteins, homodimers, heterodimers, polynucleotides, vectors, lipoplexes, such as LNPs, and/or pharmaceutical compositions, as described herein, are combined with an interleukin agonist, such as an IL-2, IL-7, IL-15, IL-10, or IL-12 agonist. Illustrative examples of IL-2 agonists that can be combined with an agent of this disclosure include without limitation proleukin (aldesleukin, IL-2); pegylated IL-2 (e.g., NKTR-214); modified variants of IL-2 (e.g., THOR-707), bempegaldesleukin, AIC-284, ALKS-4230, CUI-101, and Neo-2/15. Illustrative examples of IL-15 agonists that can be combined with an agent of this disclosure include without limitation ALT-803, NKTR-255, hetlL-15, interleukin-15/Fc fusion protein, AM-0015, NIZ-985, SO-C101, IL-15 Synthorin (pegylated 11-15), P-22339, and IL-15-PD-1 fusion protein N-809. An illustrative example of an IL-7 agonist that can be combined with an agent of this disclosure is CYT-107.

### Pharmacokinetic Enhancers

In various embodiments, the FLT3L-Fc fusion proteins, homodimers, heterodimers, polynucleotides, vectors, lipoplexes, such as LNPs, and/or pharmaceutical compositions, as described herein, are combined with a pharmacokinetic enhancer such as cobicistat and ritonavir.

### Interferons

In various embodiments, the FLT3L-Fc fusion proteins, homodimers, heterodimers, polynucleotides, vectors, lipoplexes, such as LNPs, and/or pharmaceutical compositions, as described herein, are combined with an interferon. In some embodiments, the interferon that can be combined with an agent of this disclosure is selected from interferon alfacon 1, interferon alfa 1b, interferon alfa 2a, interferon alfa 2b, pegylated interferon alfacon 1, pegylated interferon alfa 1b, pegylated interferon alfa 2a (PegIFNα-2a), and PegIFNα-2b, and combinations thereof. In some embodiments, the interferon that can be combined with an agent of this disclosure is selected from interferon alfacon 1, pegylated interferon alfa 2a (PegIFNα-2a), PegIFNα-2b, ribavirin, and combinations thereof. In some embodiments, the interferon that can be combined with an agent of this disclosure is selected from pegylated interferon alfa-2a, pegylated interferon alfa-2b, and combinations thereof.

### Immunostimulatory Agents

In various embodiments, the FLT3L-Fc fusion proteins, homodimers, heterodimers, polynucleotides, vectors, lipoplexes, such as LNPs, and/or pharmaceutical compositions, as described herein, are combined with an immunostimulatory agent, such as an oligonucleotide or an antimitotic inhibitor. In some embodiments, the immunostimulatory agent that can be combined with an agent of this disclosure is selected from fomivirsen, podofilox, imiquimod, sinecatechins, and combinations thereof.

### Additional Therapeutic Agents

In various embodiments, the FLT3L-Fc fusion proteins, homodimers, heterodimers, polynucleotides, vectors, lipoplexes, such as LNPs, and/or pharmaceutical compositions, as described herein, are combined with an additional therapeutic agent selected from the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US 20140221356 (Gilead Sciences), US 20100143301 (Gilead Sciences) and WO 2013/091096 (Boehringer Ingelheim).

In various embodiments, the FLT3L-Fc fusion proteins, homodimers, heterodimers, polynucleotides, vectors, lipoplexes, such as LNPs, and/or pharmaceutical compositions, as described herein, are combined with an additional therapeutic agent selected from besifovir, nitazoxanide, REGN2222, doravirine, sofosbuvir, velpatasvir, daclatasvir, asunaprevir, beclabuvir, FV100, and letermovir, and combinations thereof.

In various embodiments, the FLT3L-Fc fusion proteins, homodimers, heterodimers, polynucleotides, vectors, lipoplexes, such as LNPs, and/or pharmaceutical compositions, as described herein, are combined with an additional therapeutic agent selected from IFX-1, FM-201, CYNK-001, DPP4-Fc, ranpirnase, nafamostat, LB-2, AM-1, anti-viroporins, and combinations thereof.

Exemplified Combination Therapies

### Lymphoma or Leukemia Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat a lymphoma or leukemia. In some embodiments, the FLT3L-Fc fusion proteins, homodimers, heterodimers, polynucleotides, vectors, lipoplexes, such as LNPs, and/or pharmaceutical compositions, as described herein, are used in combination with one or more chemotherapy agents, radioimmunotherapy agents, and/or immunotherapy agents suitable for treating lymphoma or leukemia. Some chemotherapy agents are suitable for treating lymphoma or leukemia. These agents include aldesleukin, alvocidib, amifostine trihydrate, aminocamptothecin, antineoplaston A10, antineoplaston AS2-1, anti-thymocyte globulin, arsenic trioxide, Bcl-2 family protein inhibitor ABT-263, beta alethine, BMS-345541, bortezomib (VELCADE®), bortezomib (VELCADE®, PS-341), bryostatin 1, bulsulfan, campath-1H, carboplatin, carfilzomib (Kyprolis®), carmustine, caspofungin acetate, CC-5103, chlorambucil, CHOP (cyclophosphamide, doxorubicin, vincristine, and prednisone), cisplatin, cladribine, clofarabine, curcumin, CVP (cyclophosphamide, vincristine, and prednisone), cyclophosphamide, cyclosporine, cytarabine, denileukin diftitox, dexamethasone, docetaxel, dolastatin 10, doxorubicin, doxorubicin hydrochloride, DT-PACE (dexamethasone, thalidomide, cisplatin, doxorubicin, cyclophosphamide, and etoposide), enzastaurin, epoetin alfa, etoposide, everolimus (RAD001), FCM (fludarabine, cyclophosphamide, and mitoxantrone), FCR (fludarabine, cyclophosphamide, and rituximab), fenretinide, filgrastim, flavopiridol, fludarabine, FR (fludarabine and rituximab), geldanamycin (17 AAG), hyperCVAD (hyperfractionated cyclophosphamide, vincristine, doxorubicin, dexamethasone, methotrexate, and cytarabine), ICE (iphosphamide, carboplatin, and etoposide), ifosfamide, irinotecan hydrochloride, interferon alpha-2b, ixabepilone, lenalidomide (REVLIMID®, CC-5013), lymphokine-activated killer cells, MCP (mitoxantrone, chlorambucil, and prednisolone), melphalan, mesna, methotrexate, mitoxantrone hydrochloride, motexafin gadolinium, mycophenolate mofetil, nelarabine, obatoclax (GX15-070), oblimersen, octreotide acetate, omega-3 fatty acids, Omr-IgG-am (WHIG, Omrix), oxaliplatin, paclitaxel, palbociclib (PD0332991), pegfilgrastim, PEGylated liposomal doxorubicin hydrochloride, perifosin, prednisolone, prednisone, recombinant flt3 ligand, recombinant human thrombopoietin, recombinant interferon alfa, recombinant interleukin-11, recombinant interleukin-12, rituximab, R-CHOP (rituximab and CHOP), R-CVP (rituximab and CVP), R-FCM (rituximab and FCM), R-ICE (rituximab and ICE), and R MCP (rituximab and MCP), R-roscovitine (seliciclib, CYC202), sargramostim, sildenafil citrate, simvastatin, sirolimus, styryl sulphones, tacrolimus, tanespimycin, temsirolimus (CC1-779), thalidomide, therapeutic allogeneic lymphocytes, thiotepa, tipifarnib, vincristine, vincristine sulfate, vinorelbine ditartrate, SAHA (suberanilohydroxamic acid, or suberoyl, anilide, and hydroxamic acid), vemurafenib (Zelboraf®), venetoclax (ABT-199).

One modified approach is radioimmunotherapy, wherein a monoclonal antibody is combined with a radioisotope particle, such as indium-111, yttrium-90, and iodine-131. Examples of combination therapies include, but are not limited to, iodine-131 tositumomab (BEXXAR®), yttrium-90 ibritumomab tiuxetan (ZEVALIN®), and BEXXAR® with CHOP.

The above mentioned therapies can be supplemented or combined with stem cell transplantation or treatment. Therapeutic procedures include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, low-LET cobalt-60 gamma ray therapy, bleomycin, conventional surgery, radiation therapy, and nonmyeloablative allogeneic hematopoietic stem cell transplantation.

### Non-Hodgkin's Lymphomas Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat non-Hodgkin's lymphoma (NHL). In some embodiments, the FLT3L-Fc fusion proteins, homodimers, heterodimers, polynucleotides, vectors, lipoplexes, such as LNPs, and/or pharmaceutical compositions, as described herein, are used in combination with one or more chemotherapy agents, radioimmunotherapy agents, and/or immunotherapy agents suitable for treating NHL. Treatment of non-Hodgkin's lymphomas (NHL), especially those of B cell origin, includes using monoclonal antibodies, standard chemotherapy approaches (e.g., CHOP (cyclophosphamide, doxorubicin, vincristine, and prednisone), CVP (cyclophosphamide, vincristine, and prednisone), FCM (fludarabine, cyclophosphamide, and mitoxantrone), MCP (Mitoxantrone, Chlorambucil, Prednisolone), all optionally including rituximab (R) and the like), radioimmunotherapy, and combinations thereof, especially integration of an antibody therapy with chemotherapy.

Examples of unconjugated monoclonal antibodies for the treatment of NHL/B-cell cancers include rituximab, alemtuzumab, human or humanized anti-CD20 antibodies, lumiliximab, anti-TNF-related apoptosis-inducing ligand (anti-TRAIL), bevacizumab, galiximab, epratuzumab, SGN-40, and anti-CD74.

Examples of experimental antibody agents used in treatment of NHL/B-cell cancers include ofatumumab, ha20, PRO131921, alemtuzumab, galiximab, SGN-40, CHIR-12.12, epratuzumab, lumiliximab, apolizumab, milatuzumab, and bevacizumab.

Examples of standard regimens of chemotherapy for NHL/B-cell cancers include CHOP, FCM, CVP, MCP, R-CHOP (rituximab, cyclophosphamide, doxorubicin, vincristine, and prednisone), R-FCM, R-CVP, and R MCP.

Examples of radioimmunotherapy for NHL/B-cell cancers include yttrium-90 ibritumomab tiuxetan (ZEVALIN®) and iodine-131 tositumomab (BEXXAR®).

### Mantle Cell Lymphoma Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat mantle cell lymphoma (MCL). In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic treatments for MCL or immunotherapy or radiotherapy agents suitable for treating MCL. Therapeutic treatments for mantle cell lymphoma (MCL) include combination chemotherapies such as CHOP, hyperCVAD, and FCM. These regimens can also be supplemented with the monoclonal antibody rituximab to form combination therapies R-CHOP, hyperCVAD-R, and R-FCM. Any of the abovementioned therapies may be combined with stem cell transplantation or ICE in order to treat MCL.

An alternative approach to treating MCL is immunotherapy. One immunotherapy uses monoclonal antibodies like rituximab. Another uses cancer vaccines, such as GTOP-99, which are based on the genetic makeup of an individual patient's tumor.

A modified approach to treat MCL is radioimmunotherapy, wherein a monoclonal antibody is combined with a radioisotope particle, such as iodine-131 tositumomab (BEXXAR®) and yttrium-90 ibritumomab tiuxetan (ZEVALIN®). In another example, BEXXAR® is used in sequential treatment with CHOP.

Other approaches to treating MCL include autologous stem cell transplantation coupled with high-dose chemotherapy, administering proteasome inhibitors such as bortezomib (VELCADE® or PS-341), or administering antiangiogenesis agents such as thalidomide, especially in combination with rituximab.

Another treatment approach is administering drugs that lead to the degradation of Bcl-2 protein and increase cancer cell sensitivity to chemotherapy, such as oblimersen, in combination with other chemotherapeutic agents.

A further treatment approach includes administering mTOR inhibitors, which can lead to inhibition of cell growth and even cell death. Non-limiting examples are sirolimus, temsirolimus (TORISEL®, CCI-779), CC-115, CC-223, SF-1126, PQR-309 (bimiralisib), voxtalisib, GSK-2126458, and temsirolimus in combination with RITUXAN®, VELCADE®, or other chemotherapeutic agents.

Other recent therapies for MCL have been disclosed. Such examples include flavopiridol, palbociclib (PD0332991), R-roscovitine (selicicilib, CYC202), styryl sulphones, obatoclax (GX15-070), TRAIL, Anti-TRAIL death receptors DR4 and DR5 antibodies, temsirolimus (TORISEL®, CC1-779), everolimus (RAD001), BMS-345541, curcumin, SAHA, thalidomide, lenalidomide (REVLIMID®, CC-5013), and geldanamycin (17 AAG).

### Waldenstrom's Macroglobulinemia Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat Waldenstrom's Macroglobulinemia (WM). In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents agents suitable for treating WM. Therapeutic agents used to treat Waldenstrom's Macroglobulinemia (WM) include aldesleukin, alemtuzumab, alvocidib, amifostine trihydrate, aminocamptothecin, antineoplaston A10, antineoplaston AS2-1, anti-thymocyte globulin, arsenic trioxide, autologous human tumor-derived HSPPC-96, Bcl-2 family protein inhibitor ABT-263, beta alethine, bortezomib (VELCADE®), bryostatin 1, busulfan, campath-1H, carboplatin, carmustine, caspofungin acetate, CC-5103, cisplatin, clofarabine, cyclophosphamide, cyclosporine, cytarabine, denileukin diftitox, dexamethasone, docetaxel, dolastatin 10, doxorubicin hydrochloride, DT-PACE, enzastaurin, epoetin alfa, epratuzumab (hLL2-anti-CD22 humanized antibody), etoposide, everolimus, fenretinide, filgrastim, fludarabine, ibrutinib, ifosfamide, indium-111 monoclonal antibody MN-14, iodine-131 tositumomab, irinotecan hydrochloride, ixabepilone, lymphokine-activated killer cells, melphalan, mesna, methotrexate, mitoxantrone hydrochloride, monoclonal antibody CD19 (such as tisagenlecleucel-T, CART-19, CTL-019), monoclonal antibody CD20, motexafin gadolinium, mycophenolate mofetil, nelarabine, oblimersen, octreotide acetate, omega-3 fatty acids, oxaliplatin, paclitaxel, pegfilgrastim, PEGylated liposomal doxorubicin hydrochloride, pentostatin, perifosine, prednisone, recombinant flt3 ligand, recombinant human thrombopoietin, recombinant interferon alfa, recombinant interleukin-11, recombinant interleukin-12, rituximab, sargramostim, sildenafil citrate (VIAGRA®), simvastatin, sirolimus, tacrolimus, tanespimycin, thalidomide, therapeutic allogeneic lymphocytes, thiotepa, tipifarnib, tositumomab, ulocuplumab, veltuzumab, vincristine sulfate, vinorelbine ditartrate, vorinostat, WT1 126-134 peptide vaccine, WT-1 analog peptide vaccine, yttrium-90 ibritumomab tiuxetan, yttrium-90 humanized epratuzumab, and any combination thereof.

Examples of therapeutic procedures used to treat WM include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme techniques, low-LET cobalt-60 gamma ray therapy, bleomycin, conventional surgery, radiation therapy, and nonmyeloablative allogeneic hematopoietic stem cell transplantation.

### Diffuse Large B-cell Lymphoma Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat diffuse large B-cell lymphoma (DLBCL). In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating DLBCL. Therapeutic agents used to treat diffuse large B-cell lymphoma (DLBCL) include cyclophosphamide, doxorubicin, vincristine, prednisone, anti-CD20 monoclonal antibodies, etoposide, bleomycin, many of the agents listed for WM, and any combination thereof, such as ICE and RICE.

### Chronic Lymphocytic Leukemia Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat chronic lymphocytic leukemia (CLL). In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating CLL. Examples of therapeutic agents used to treat chronic lymphocytic leukemia (CLL) include chlorambucil, cyclophosphamide, fludarabine, pentostatin, cladribine, doxorubicin, vincristine, prednisone, prednisolone, alemtuzumab, many of the agents listed for WM, and combination chemotherapy and chemoimmunotherapy, including the following common combination regimens: CVP, R-CVP, ICE, R-ICE, FCR, and FR.

### Myelofibrosis Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat myelofibrosis. In some embodiments, the FLT3L-Fc fusion proteins, homodimers, heterodimers, polynucleotides, vectors, lipoplexes, such as LNPs, and/or pharmaceutical compositions, as described herein, are used in combination with one or more myelofibrosis inhibiting agents. Myelofibrosis inhibiting agents include, but are not limited to, hedgehog inhibitors, hi stone deacetylase (HDAC) inhibitors, and tyrosine kinase inhibitors. Non-limiting examples of hedgehog inhibitors are saridegib and vismodegib. Examples of HDAC inhibitors include, but are not limited to, pracinostat and panobinostat. Non-limiting examples of tyrosine kinase inhibitors are lestaurtinib, bosutinib, imatinib, gilteritinib, radotinib, and cabozantinib.

### Hyperproliferative Disorder Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat a hyperproliferative disorder. In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating hyperproliferative disorders. Gemcitabine, nab-paclitaxel, and gemcitabine/nab-paclitaxel may be used with a JAK inhibitor and/or PI3Kδ inhibitor to treat hyperproliferative disorders.

### Bladder Cancer Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat bladder cancer. In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating bladder cancer. Therapeutic agents used to treat bladder cancer include atezolizumab, carboplatin, cisplatin, docetaxel, doxorubicin, fluorouracil (5-FU), gemcitabine, idosfamide, Interferon alfa-2b, methotrexate, mitomycin, nab-paclitaxel, paclitaxel, pemetrexed, sacituzumab govitecan, thiotepa, vinblastine, and any combination thereof. In some embodiments, the FLT3L-Fc fusion proteins, homodimers, heterodimers, polynucleotides, vectors, lipoplexes, such as LNPs, and/or pharmaceutical compositions, as described herein, are used in combination with sacituzumab govitecan for treating bladder cancer. In some embodiments, the FLT3L-Fc fusion proteins, homodimers, heterodimers, polynucleotides, vectors, lipoplexes, such as LNPs, and/or pharmaceutical compositions, as described herein, are used in combination with atezolizumab for treating bladder cancer.

### Breast Cancer Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat breast cancer. In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating breast cancer. Therapeutic agents used to treat breast cancer include albumin-bound paclitaxel, anastrozole, capecitabine, carboplatin, cisplatin, cyclophosphamide, docetaxel, doxorubicin, epirubicin, everolimus, exemestane, fluorouracil, fulvestrant, gemcitabine, Ixabepilone, lapatinib, Letrozole, methotrexate, mitoxantrone, paclitaxel, pegylated liposomal doxorubicin, pertuzumab, tamoxifen, toremifene, trastuzumab, vinorelbine, and any combinations thereof.

### Triple Negative Breast Cancer Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat triple negative breast cancer. In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating triple negative breast cancer. Therapeutic agents used to treat triple negative breast cancer include cyclophosphamide, docetaxel, doxorubicin, epirubicin, fluorouracil, paclitaxel, and combinations thereof.

### Colorectal Cancer Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat colorectal cancer. In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating colorectal cancer. Therapeutic agents used to treat colorectal cancer include bevacizumab, capecitabine, cetuximab, fluorouracil, irinotecan, leucovorin, oxaliplatin, panitumumab, ziv-aflibercept, and any combinations thereof.

### Castration-Resistant Prostate Cancer Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat castration resistant prostate cancer. In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating castration-resistant prostate cancer. Therapeutic agents used to treat castration-resistant prostate cancer include abiraterone, cabazitaxel, docetaxel, enzalutamide, prednisone, sipuleucel-T, and any combinations thereof.

### Esophageal and Esophagogastric Junction Cancer Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat esophageal and esophagogastric junction cancer. In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating esophageal and esophagogastric junction cancer. Therapeutic agents used to treat esophageal and esophagogastric junction cancer include capecitabine, carboplatin, cisplatin, docetaxel, epirubicin, fluoropyrimidine, fluorouracil, irinotecan, leucovorin, oxaliplatin, paclitaxel, ramucirumab, trastuzumab, and any combinations thereof.

### Gastric Cancer Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat gastric cancer. In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating gastric cancer. Therapeutic agents used to treat gastric cancer include capecitabine, carboplatin, cisplatin, docetaxel, epirubicin, fluoropyrimidine, fluorouracil, Irinotecan, leucovorin, mitomycin, oxaliplatin, paclitaxel, ramucirumab, trastuzumab, and any combinations thereof

### Head and Neck Cancer Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat head & neck cancer. In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating head & neck cancer. Therapeutic agents used to treat head & neck cancer include afatinib, bleomycin, capecitabine, carboplatin, cetuximab, cisplatin, docetaxel, fluorouracil, gemcitabine, hydroxyurea, methotrexate, nivolumab, paclitaxel, pembrolizumab, vinorelbine, and any combinations thereof.

### Hepatobiliary Cancer Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat hepatobiliary cancer. In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating hepatobiliary cancer. Therapeutic agents used to treat hepatobiliary cancer include capecitabine, cisplatin, fluoropyrimidine, 5-fluorourcil, gemecitabine, oxaliplatin, sorafenib, and any combinations thereof.

### Hepatocellular Carcinoma Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat hepatocellular carcinoma. In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating hepatocellular carcinoma. Therapeutic agents used to treat hepatocellular carcinoma include capecitabine, doxorubicin, gemcitabine, sorafenib, and any combinations thereof.

### Non-Small Cell Lung Cancer Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat non-small cell lung cancer (NSCLC). In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating NSCLC. Therapeutic agents used to treat non-small cell lung cancer (NSCLC) include afatinib, albumin-bound paclitaxel, alectinib, atezolizumab, bevacizumab, bevacizumab, cabozantinib, carboplatin, cisplatin, crizotinib, dabrafenib, docetaxel, erlotinib, etoposide, gemcitabine, nivolumab, paclitaxel, pembrolizumab, pemetrexed, ramucirumab, tiragolumab, trametinib, trastuzumab, vandetanib, vemurafenib, vibostolimab, vinblastine, vinorelbine, and any combinations thereof. In some embodiments, the FLT3L-Fc fusion proteins, homodimers, heterodimers, polynucleotides, vectors, lipoplexes, such as LNPs, and/or pharmaceutical compositions, as described herein, are used in combination with pembrolizumab to treat NSCLC. In some embodiments, the FLT3L-Fc fusion proteins, homodimers, heterodimers, polynucleotides, vectors, lipoplexes, such as LNPs, and/or pharmaceutical compositions, as described herein, are used in combination with pembrolizumab and vibostolimab to treat NSCLC. In some embodiments, the FLT3L-Fc fusion proteins, homodimers, heterodimers, polynucleotides, vectors, lipoplexes, such as LNPs, and/or pharmaceutical compositions, as described herein, are used in combination with atezolizumab to treat NSCLC. In some embodiments, the FLT3L-Fc fusion proteins, homodimers, heterodimers, polynucleotides, vectors, lipoplexes, such as LNPs, and/or pharmaceutical compositions, as described herein, are used in combination with atezolizumab and tiragolumab to treat NSCLC.

### Small Cell Lung Cancer Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat small cell lung cancer (SCLC). In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating SCLC. Therapeutic agents used to treat small cell lung cancer (SCLC) include bendamustime, carboplatin, cisplatin, cyclophosphamide, docetaxel, doxorubicin, etoposide, gemcitabine, ipillimumab, irinotecan, nivolumab, paclitaxel, temozolomide, topotecan, vincristine, vinorelbine, and any combinations thereof.

### Melanoma Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat melanoma. In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating melanoma. Therapeutic agents used to treat melanoma cancer include albumin bound paclitaxel, carboplatin, cisplatin, cobiemtinib, dabrafenib, dacrabazine, IL-2, imatinib, interferon alfa-2b, ipilimumab, nitrosourea, nivolumab, paclitaxel, pembrolizumab, pilimumab, temozolomide, trametinib, vemurafenib, vinblastine, and any combinations thereof.

### Ovarian Cancer Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat ovarian cancer. In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating ovarian cancer. Therapeutic agents used to treat ovarian cancer include 5-flourouracil, albumin bound paclitaxel, altretamine, anastrozole, bevacizumab, capecitabine, carboplatin, cisplatin, cyclophosphamide, docetaxel, doxorubicin, etoposide, exemestane, gemcitabine, ifosfamide, irinotecan, letrozole, leuprolide acetate, liposomal doxorubicin, megestrol acetate, melphalan, olaparib, oxaliplatin, paclitaxel, Pazopanib, pemetrexed, tamoxifen, topotecan, vinorelbine, and any combinations thereof.

### Pancreatic Cancer Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat pancreatic cancer. In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating pancreatic cancer. Therapeutic agents used to treat pancreatic cancer include 5-fluorourcil, albumin-bound paclitaxel, capecitabine, cisplatin, docetaxel, erlotinib, fluoropyrimidine, gemcitabine, irinotecan, leucovorin, oxaliplatin, paclitaxel, and any combinations thereof.

### Renal Cell Carcinoma Combination Therapy

In some embodiments, the FLT3L-Fc fusion proteins, homodimers, heterodimers, polynucleotides, vectors, lipoplexes, such as LNPs, and/or pharmaceutical compositions, as described herein, are used to treat renal cell carcinoma. In some embodiments, the FLT3L-Fc fusion proteins, homodimers, heterodimers, polynucleotides, vectors, lipoplexes, such as LNPs, and/or pharmaceutical compositions, as described herein, are used in combination with one or more therapeutic agents suitable for treating renal cell carcinoma. Therapeutic agents used to treat renal cell carcinoma include axitinib, bevacizumab, cabozantinib, erlotinib, everolimus, levantinib, nivolumab, pazopanib, sorafenib, sunitinib, temsirolimus, and any combinations thereof

9. Kits

Further provided are kits comprising one or more containers comprising one or more unitary doses of a FLT3L-Fc fusion protein, as described herein, a homodimer or heterodimer comprising such fusion protein, a polynucleotide encoding such fusion protein, a vector or lipoplex, such as a lipid nanoparticle (LNP) comprising such polynucleotide, or pharmaceutical composition comprising such fusion protein or polynucleotide. In some embodiments, the kits comprise two or more unitary doses of the FLT3L-Fc fusion protein, the homodimer or heterodimer comprising such fusion protein, the polynucleotide encoding such fusion protein, the vector or lipoplex, such as a lipid nanoparticle (LNP) comprising such polynucleotide, or pharmaceutical composition comprising such fusion protein or polynucleotide, in two or more containers. In some embodiments, the kit comprises one or more unitary doses of the FLT3L-Fc fusion protein, the homodimer or heterodimer comprising such fusion protein, the polynucleotide encoding such fusion protein, the vector or lipoplex, such as a lipid nanoparticle (LNP) comprising such polynucleotide, or pharmaceutical composition comprising such fusion protein or polynucleotide and one or more (e.g., one, two, three, one or two, or one to three) additional therapeutic agents in separate containers. The one or more additional therapeutic agents (e.g., for vaccination and/or for treating cancer or a viral infection) are as described above and herein. In some embodiments, the kits comprise two or more unitary doses wherein the unitary doses are the same. In some embodiments, the kits comprise two or more unitary doses, wherein the unitary doses are different.

In one embodiment, the kit comprises one or more pharmaceutical packs comprising one or more containers (e.g., vials, ampules, pre-loaded syringes) containing one or more of the ingredients of the pharmaceutical compositions described herein, such as the FLT3L-Fc fusion protein, the homodimer or heterodimer comprising such fusion protein, the polynucleotide encoding such fusion protein, the vector or lipoplex, such as a lipid nanoparticle (LNP), comprising such polynucleotide, or pharmaceutical composition comprising such fusion protein or polynucleotide, as provided herein. In some instances, the kits contain a pharmaceutical composition described herein. In some embodiments, the kit comprises one or more containers comprising the FLT3L-Fc fusion protein, the homodimer or heterodimer comprising such fusion protein, the polynucleotide encoding such fusion protein, the vector or lipoplex, such as a lipid nanoparticle (LNP), comprising such polynucleotide, or pharmaceutical composition comprising such fusion protein or polynucleotide, in an aqueous solution. In some embodiments, the aqueous solution comprises the FLT3L-Fc fusion protein, the homodimer or heterodimer comprising such fusion protein, or pharmaceutical composition comprising such fusion protein, at a concentration in the range of about 1 mg/ml to about 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml or 20 mg/ml. In some embodiments, the kit comprises one or more containers comprising the FLT3L-Fc fusion protein, the homodimer or heterodimer comprising such fusion protein, the polynucleotide encoding such fusion protein, the vector or lipoplex, such as a lipid nanoparticle (LNP), comprising such polynucleotide, or pharmaceutical composition comprising such fusion protein or polynucleotide, in lyophilized form.

Optionally associated with such container(s) can be a notice in the form prescribed by a governmental agency regulating the manufacture, use or sale of pharmaceuticals or biological products, which notice reflects approval by the agency of manufacture, use or sale for human administration.

### EXAMPLES

The following examples are offered to illustrate, but not to limit the claimed invention.

### Example 1

### In Vitro Potency of Different FLT3 Agonist Modalities

In this example, we compared the in vitro potency of FLT3 agonists of different modalities, including recombinant ligand, FLT3 ligand-Fc fusion protein, and anti-mouse FLT3 agonist antibody (comparator 1). We tested in vitro potency employing a M1 IL-6 Secretion Assay.

### Methods

M1 IL-6 Secretion Assay: Murine myeloid leukemic M1 cells (American Type Culture Collection (ATCC), TIB-192) were collected from culture, counted, and resuspended to 0.5×10⁶ cells/ml with serum-free RPMI1640. In a 96-well U-bottom tissue culture plate, 100 μl of resuspended cells (50,000 cells) was added to each well, then 50 μl of 4× test article was added to each well and 50 μl of serum-free RPMI was added into the wells for a final volume of 200 μl per well. Cells were incubated overnight at 37° C. The next day, cells were spun down at 500 g for 5 min at ambient temperatures. Supernatants were then collected for mouse IL-6 quantification (Meso Scale Discovery (MSD), Cat: K152AKB-1) which was performed by following the manufacturer's protocol. EC50 and Emax values of each sample were determined by plotting the concentration of the FLT3 agonist compound against the IL-6 supernatant concentration (pg/mL) and fit to a four parameter logistic (4PL) regression curve.

### Results

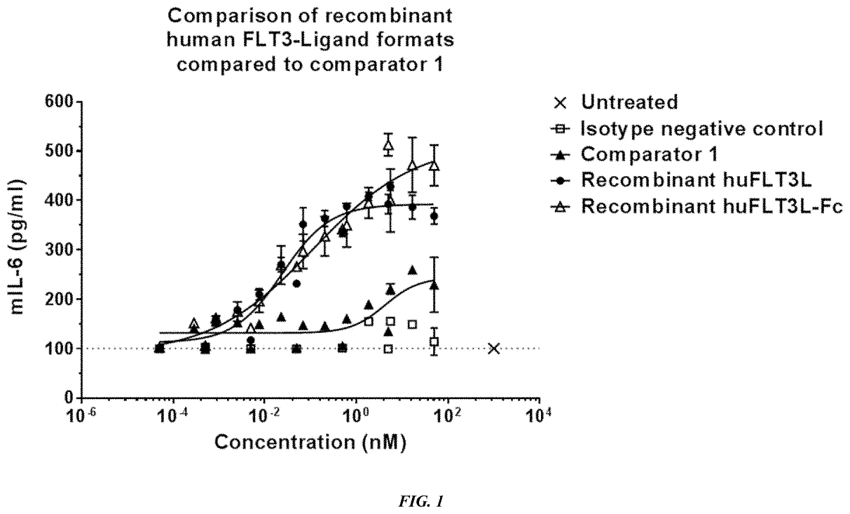

The data demonstrated that recombinant FLT3-ligand recombinant FLT3-ligand Fc fusion protein were superior to FLT3 agonist antibody (comparator 1) in activating M1 cells to produce IL-6 in a dose dependent manner. These data also demonstrated that human FLT3-ligand proteins can potently activate murine FLT3. These results are summarized in Table 1 and depicted in FIG. 1.

TABLE 1
EC50 and Emax values for Recombinant huFlt3L, Recombinant huFLT3L-Fc and
Comparator 1 in the M1 IL-6 release assay

| | anti-mouse FLT3 | | |
|---|---|---|---|
| | agonist antibody | Recombinant | Recombinant |
| | (Comparator 1) | huFlt3L-Fc | huFLT3L |
| EC50 (nM) | 4.188 | 0.117 | 0.024 |
| Emax (pg/ml) | 244.1 | 516.6 | 392.3 |

The results guided us to further pursue a FLT3-ligand Fc fusion protein as a FLT3 agonist.

### Example 2

### In Vitro Potency of FLT3L-Fc Fusion Proteins Having Different IgG Backbones

In this example, we compared the in vitro potency of different FLT3-Ligand Fc fusion protein variants: one having a hingeless human IgG1 backbone (SEQ ID NO:1) and a second having a human IgG1 backbone (SEQ ID NO:21). For this comparison, we tested the in vitro potency employing an AML5 Proliferation Assay.

### Methods

AML5 Proliferation Assay: AML5 cells (Deutsche Sammlung von Mikroorganismen and Zellkulturen (DSMZ), ACC247) were starved 0/N (18-24h) with serum free MEM-α. The next day, using a 96-well white opaque half area flat bottom TC plates (Costar, 3688), 25,000 cells per well were stimulated with the various compounds for 72h. Plates were sealed with a breathable plate sealer (Sigma, Z380059-1PAK) during the incubation. After incubation, proliferation was assessed through CellTiter Glo assay (Promega, G7571) using manufacturer's recommendations. Luminescence signals were measured using a SpectraMax plate reader. EC50 value of each sample was determined by plotting the concentration of the compound against the luminescence signal and fit to a 4PL curve.

### Results

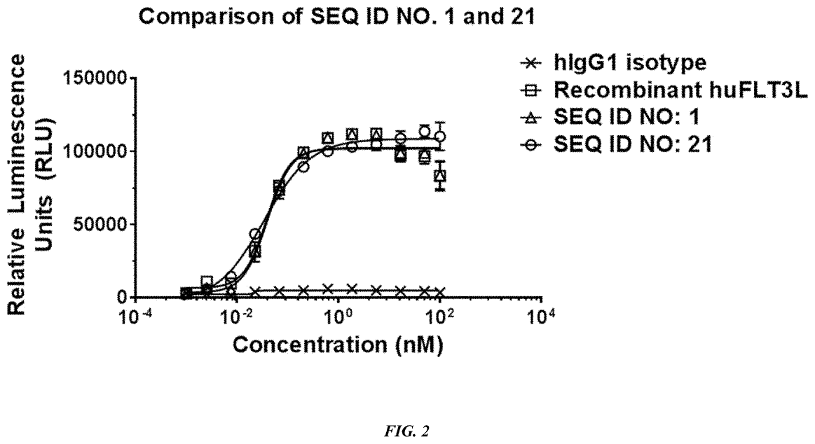

The data demonstrated that the potency of FLT3-ligand Fc fusion with hingeless IgG1 (SEQ ID NO:1) and FLT3-ligand Fc fusion with IgG1 (SEQ ID NO:21) was similar to that of recombinant human FLT3 ligand in inducing FLT3-dependent proliferation in AML5 cells, with EC50 values ranging between 0.035-0.04 nM. The results are summarized in Table 2 and depicted in FIG. 2.

TABLE 2
EC50 values for Inducing Proliferation of AML5 Cells by FLT3L-Fc
Variants Having Different IgG Backbones
EC50 (nM)

| recombinant | hingeless IgG1 | IgG1 |
|---|---|---|
| huFLT3L | SEQ ID NO: 1 | SEQ ID NO: 21 |
| 0.039 | 0.040 | 0.035 |

### Example 3

### In Vitro Potency of FLT3L-Fc Variants Having Mutations in the FLT3L EC Domain

In this example, we compared the in vitro potency of human FLT3-ligand hingeless human IgG1 fusion proteins containing different FLT3-ligand gain-of-fusion mutations. We tested the in vitro potency FLT3L-Fc fusion protein variants having mutations in the FLT3L extracellular (EC) domain (H8Y and/or K84E) by employing an AML5 Proliferation Assay. The methods are as described above in Example 2.

### Results

The data demonstrated that the potency of FLT3-ligand Fc fusion protein variants with gain-of-function mutations in the FLT3L extracellular domain (H8Y and/or K84E; SEQ ID NOs: 22, 23 and 24) in inducing FLT3-dependent proliferation in AML5 cells was approximately 2.5× fold higher than that of human FLT3-ligand Fc fusion protein (SEQ ID NO:1). The results are summarized in Table 3 and depicted in FIG. 3.

TABLE 3
EC50 values for Inducing Proliferation of AML5 Cells by FLT3L-Fc
Variants Having Mutations in the FLT3L EC Domain
EC50 (nM)

| | hFLT3L ECD | hFLT3L ECD | hFLT3L ECD |
|---|---|---|---|
| hFLT3L ECD | (H8Y) | (K84E) | (H8Y/K84E) |
| SEQ ID NO: 1 | SEQ ID NO: 22 | SEQ ID NO: 23 | SEQ ID NO: 24 |
| 0.034 | 0.014 | 0.013 | 0.013 |

### Example 4

### In Vitro Potency of Murine Surrogate FLT3-Ligand Fc Fusion Variants

In this example, we compared the in vitro potency of human FLT3-ligand hingeless human IgG1 fusion protein with two murine surrogate FLT3-ligand Fc fusion proteins. These murine surrogate proteins contained the wild type murine FLT3-ligand extracellular region fused to a L234A/L235A/P329G variant of murine IgG2a Fc region (IgG2a-LALA-PG), or, a C136S variant of murine FLT3-ligand extracellular region fused to the same Fc, where the C136S mutation was incorporated to eliminate an unpaired cysteine liability. We tested the in vitro potency employing an AML5 Proliferation Assay. The methods are as described above in Example 2.

### Results

The data demonstrated that the potency of murine surrogate FLT3-ligand Fc fusion proteins (SEQ ID NOs: 19 and 20) in inducing human FLT3-dependent proliferation in AML5 cells is similar to that of human FLT3-ligand Fc fusion protein (SEQ ID NO:1), with EC50 values ranging between 0.171-0.078 nM. The results are summarized in Table 4 and depicted in FIG. 4.

TABLE 4
EC50 values for Inducing Proliferation of AML5 Cells by Murine
Surrogate FLT3-Ligand Fc Fusion Variants
EC50 (nM)

| SEQ ID NO: 1 | SEQ ID NO: 19 | SEQ ID NO: 20 |
|---|---|---|
| 0.078 | 0.171 | 0.115 |

Based on these assay results and reduced risk of disulfide-mediated aggregation, we proceeded with using the murine surrogate FLT3-ligand Fc fusion variant of SEQ ID NO:20 in mouse preclinical models.

### Example 5

### In Vitro Potency of Different FLT3L-Fc Proteins

In this example, we compared the in vitro potency of eight different human FLT3-ligand human Fc fusion proteins. We tested the in vitro potency employing an AML5 Proliferation Assay. The methods are as described above in Example 2.

### Results

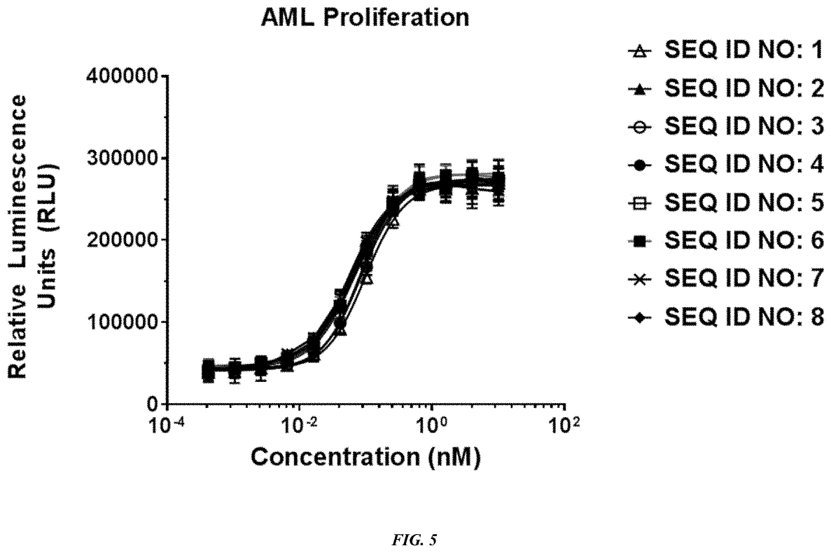

We tested the potency in inducing FLT3-dependent proliferation in AML5 cells of the eight human FLT3-ligand Fc fusion protein variants with different Fc regions, or containing modifications in the FLT3-ligand derived sequence (SEQ ID NOs: 1-8). The eight FLT3L-Fc variants tested are as follows: human FLT3-ligand human hingeless IgG1 fusion protein (SEQ ID NO:1), human FLT3-ligand (Δ5 amino acid) human hingeless IgG1 fusion protein (SEQ ID NO:2), human FLT3-ligand human IgG4 (S228P/L235E) fusion protein (SEQ ID NO:3), human FLT3-ligand human IgG4 (S228P/F234A/L235A) fusion protein (SEQ ID NO:4), human FLT3-ligand (S128A/S151A) human hingeless IgG1 fusion protein (SEQ ID NO:5), human FLT3-ligand (Δ5 amino acid) human IgG4 (S228P/F234A/L235A) fusion protein (SEQ ID NO:6), human FLT3-ligand (Δ10 amino acid) human hingeless IgG1 fusion protein (SEQ ID NO:7), or human FLT3-ligand (Δ10 amino acid) human IgG4 (S228P/F234A/L235A) fusion protein (SEQ ID NO:8). The resulting EC50 values ranged between 0.071-0.088 nM. The results are summarized in Table 5 and depicted in FIG. 5.

TABLE 5
EC50 Values for SEQ ID NOs: 1-8 in an
AML5 Proliferation Assay

| FLT3L-Fc Variant | |
|---|---|
| SEQ ID NO: | EC50 (nM) |
| 1 | 0.080 |
| 2 | 0.083 |
| 3 | 0.088 |
| 4 | 0.073 |
| 5 | 0.078 |
| 6 | 0.078 |
| 7 | 0.071 |
| 8 | 0.075 |

### Example 6

### In Vitro FLT3 Binding of Different FLT3L-Fc Fusion Proteins

In this example, we compared the in vitro binding to human recombinant FLT3 of eight different human FLT3-ligand human Fc fusion protein variants. We tested the in vitro FLT3 binding employing an enzyme-linked immunosorbent assay (ELISA).

### Methods

Flt3L-Fc fusion protein constructs were serially diluted and added to 96-well nickel plates (Pierce) coated with his-tagged recombinant human Flt3 receptor (Sino Biologicals). Bound Flt3L-Fc was detected using a goat anti-human (H+L) polyclonal antibody conjugated to horseradish peroxidase (Jackson Immunoresearch). Signal was developed using TMB substrate then quenched prior to reading absorbance at 450 nm on a SpectraMax plate reader. Flt3L-Fc concentration was plotted against signal and fit to a 4PL curve to determine the EC50 value of each construct.

### Results

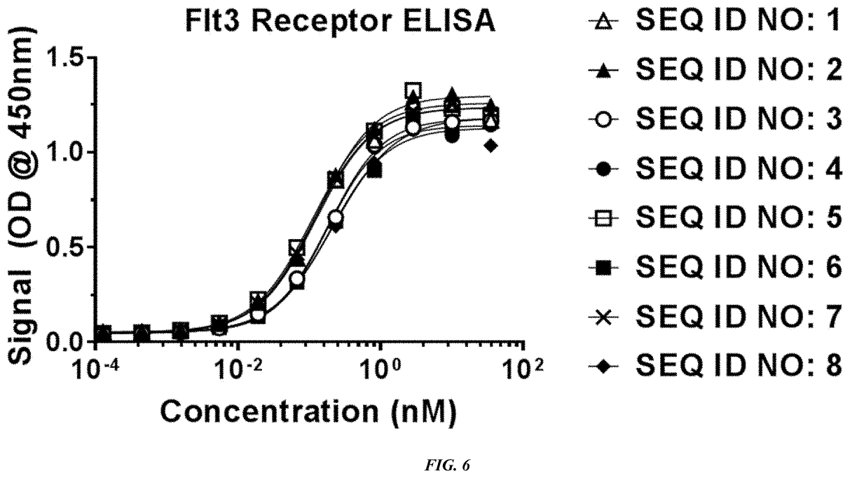

These data demonstrated that binding to human FLT3 receptor was similar for FLT3-ligand Fc fusion proteins of the same Fc isotype (IgG1, SEQ ID NOs: 1, 2, 5, 7; or IgG4, SEQ ID NOs: 3, 4, 6, 8). EC50 values ranged between 0.11-0.13 nM for IgG1 constructs, 0.18-0.22 nM for IgG4 constructs. These data also demonstrated that short truncations at the C-terminus of the FLT3-ligand portion (SEQ ID NOs: 2, 6, 7, 8) or mutations that eliminate N-linked glycans in FLT3-ligand (SEQ ID NO: 5) also had negligible effect on binding to FLT3. The results are summarized in Table 6 and depicted in FIG. 6.

TABLE 6
EC50 Values for FLT3L-Fc SEQ ID NOs: 1-8
Binding to Human Recombinant FLT3

| FLT3L-Fc Variant | |
|---|---|
| SEQ ID NO: | EC50 (nM) |
| 1 | 0.12 |
| 2 | 0.13 |
| 3 | 0.18 |
| 4 | 0.18 |
| 5 | 0.11 |
| 6 | 0.22 |
| 7 | 0.12 |
| 8 | 0.20 |

### Example 7

### In Vitro FcRn Binding of Different FLT3L-Fc Fusion Proteins

In this example, we compared the in vitro binding of eight different human FLT3-ligand human Fc fusion proteins to human recombinant FcRn. We tested the in vitro FLT3 binding employing an enzyme-linked immunosorbent assay (ELISA).

### Methods

Flt3L-Fc constructs were serially diluted and added to 96-well plates coated with recombinant human FcRn. The bound Flt3L-Fc was detected using a donkey anti-human (H+L) antibody conjugated to horseradish peroxidase (Jackson Immunoresearch). Signal was developed using TMB substrate then quenched prior to reading absorbance at 450-650 nm on a SpectraMax plate reader. Flt3L-Fc concentration was plotted against signal and fit to a 4PL curve. A full length IgG1 and IgG4 isotype was included in the initial experiment as Fc isotype controls.

### Results

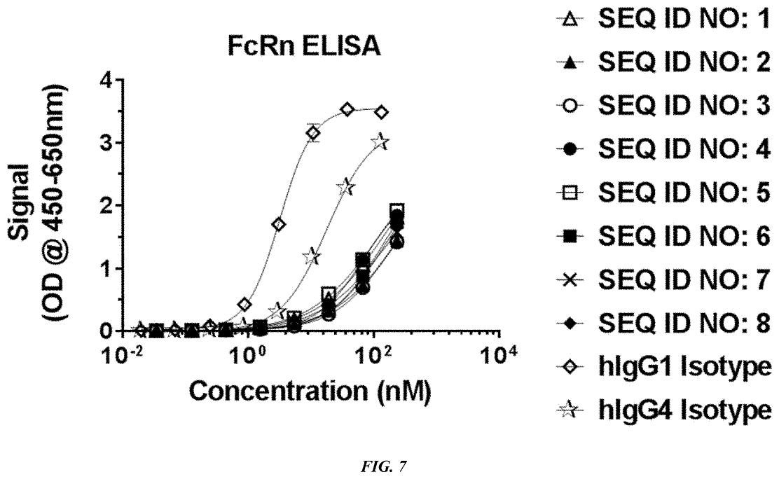

These data demonstrated that binding to human FcRn in this assay was weaker for the eight human FLT3-ligand Fc fusion proteins with different Fc variants (SEQ ID NOs: 1-8) compared to the human IgG1 and IgG4 isotype antibody controls, but relatively similar to each other. EC50 values for the FLT3L-Fc protein variant samples summarized in Table 7 are only estimates, as none of the FLT3L-Fc variants of SEQ ID NOs: 1-8 demonstrated saturating signal at the highest concentration tested. The results are also depicted in FIG. 7.

TABLE 7
Estimated EC50 values for FLT3L-Fc Variants SEQ ID NOs: 1-8
Binding to Human Recombinant FeRn

| FLT3L-Fc Variant | |
|---|---|
| SEQ ID NO: | EC50 (nM) |
| 1 | 150.70 |
| 2 | 300.40 |
| 3 | 183.30 |
| 4 | 68.76 |
| 5 | 132.70 |
| 6 | 136.50 |
| 7 | 157.90 |
| 8 | 139.90 |
| hIgG1 Isotype | 4.46 |
| hIgG4 Isotype | 25.86 |

### Example 8

### Ability of FLT3L-Fc Variants to Compete for Binding to Human FcγRI

In this example, we compared the in vitro ability of eight different human FLT3-ligand human Fc fusion proteins to compete with a human IgG molecule for binding to human recombinant FcγRI. To evaluate the ability to compete for binding to FcγRI, we employed an amplified luminescent proximity homogeneous assay (AlphaScreen® by Perkin Elmer).

### Methods

Serial dilutions of the Flt3L-Fc constructs were added to 96-well plates containing biotinylated FcγRI protein (Sino Biological). Human IgG acceptor beads (Perkin Elmer) were added to the plate, followed by streptavidin donor beads (Perkin Elmer). Acceptor beads contain thioxene derivatives. Donor beads contain a photosensitizer, phthalocyanine, which converts ambient oxygen to an excited and reactive form of 02, singlet oxygen (molecular oxygen with a single excited electron), upon illumination at 680 nm. If an acceptor bead is within 200 nm of a donor bead, energy is transferred from the singlet oxygen to thioxene derivatives within the acceptor bead, subsequently culminating in light production at 520-620 nm. Signal was measured on an EnVision™ plate reader (Perkin Elmer). Flt3L-Fc concentration was plotted against signal and fit to a 4PL curve. Full length IgG1 and IgG4 molecules were included on each plate as Fc isotype controls.

### Results

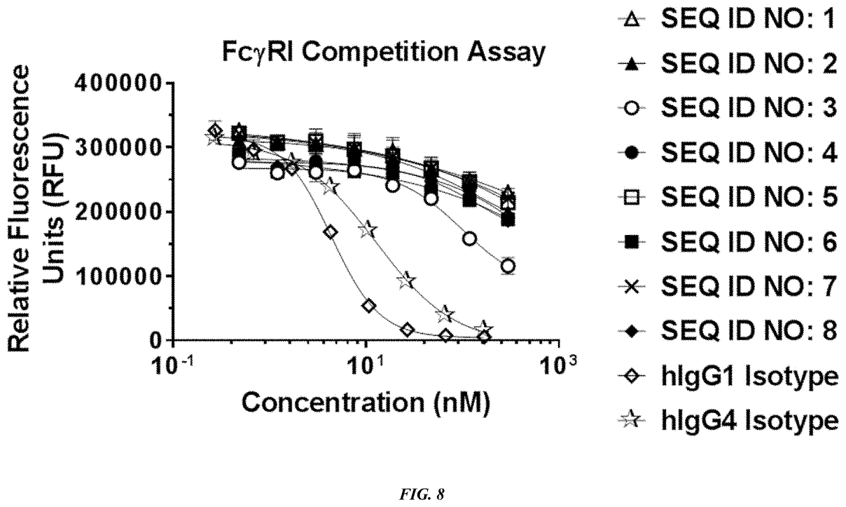

These data demonstrated that none of the eight human FLT3-ligand Fc fusion proteins with different Fc variants (SEQ ID NOs: 1-8) could fully compete with human IgG for binding to FcγRT at the highest concentrations tested. Both the human IgG1 and IgG4 isotype antibody controls demonstrated complete dose-response curves, with the IgG4 isotype showing reduced competition compared to the IgG1. The results are summarized in Table 8 and depicted in FIG. 8.

TABLE 8
EC50 Values for the Ability of FLT3L-Fc
Variants to Compete for Binding to FcγRI

| FLT3L-Fc Variant | |
|---|---|
| SEQ ID NO: | EC50 (nM) |
| 1 | NA |
| 2 | NA |
| 3 | NA |
| 4 | NA |
| 5 | NA |
| 6 | NA |
| 7 | NA |
| 8 | NA |
| hIgG1 Isotype | 4.44 |
| hIgG4 Isotype | 13.62 |

### Example 9

### Ability of FLT3L-Fc Variants to Compete for Binding to Human FcγRIIIa

In this example, we compared the in vitro ability of eight different human FLT3-ligand human Fc fusion proteins to compete for binding of human recombinant FcγRIIIa (V-variant) with a human IgG molecule. To evaluate the ability to compete for binding to FcγRIIIa, we employed an AlphaScreen® by Perkin Elmer. The methods are analogous to those described in Example 8.

### Methods

Serial dilutions of the Flt3L-Fc constructs were added to 96-well plates containing biotinylated FcγRIIIa (Val 176 variant) protein (Sino Biological). Human IgG acceptor beads (Perkin Elmer) were added to the plate, followed by streptavidin donor beads (Perkin Elmer), and signal was measured on an EnVision™ plate reader. Flt3L-Fc concentration was plotted against signal and fit to a 4PL curve. Full length IgG1 and IgG4 molecules were included on each plate as Fc isotype controls.

### Results

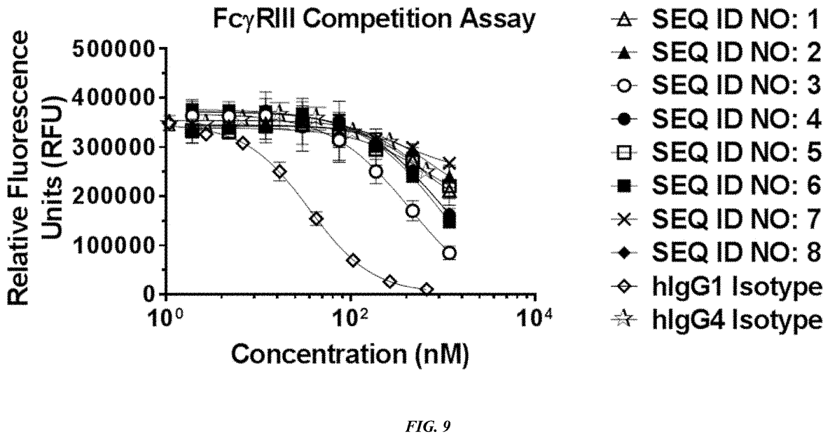

These data demonstrated that none of the eight human FLT3-ligand Fc fusion proteins with different Fc variants (SEQ ID NOs: 1-8) could fully compete with human IgG for binding to FcγRIIIa (Val176 variant) at the highest concentrations tested. Only human IgG1 isotype control demonstrated a complete dose-response curve. The results are summarized in Table 9 and depicted in FIG. 9.

TABLE 9
EC50 Values for the Ability of FLT3L-Fc
Variants to Compete for Binding to FcγRIIIa

| FLT3L-Fc Variant | |
|---|---|
| SEQ ID NO: | EC50 (nM) |
| 1 | NA |
| 2 | NA |
| 3 | NA |
| 4 | NA |
| 5 | NA |
| 6 | NA |
| 7 | NA |
| 8 | NA |
| hIgG1 Isotype | 32.09 |
| hIgG4 Isotype | 325.90 |

### Example 10

### In Vitro Binding of FLT3L-Fc Variants to Human C1q

In this example, we compared the in vitro binding of eight different human FLT3-ligand human Fc fusion proteins to human recombinant complement protein, C1q. To evaluate binding to C1q, we employed an ELISA.

### Methods

Serial dilutions of the Flt3L-Fc constructs were immobilized onto 96-well plates, followed by incubation with the recombinant human C1q protein (Fitzgerald). Binding was detected using a sheep anti-C1q antibody conjugated to horseradish peroxidase (BioRad). Signal was developed using TMB substrate then quenched prior to reading absorbance at 450-650 nm on a SpectraMax plate reader. Flt3L-Fc concentration was plotted against signal and fit to a 4PL curve. Full length IgG1 and IgG4 molecules were included on each plate as Fc isotype controls.

### Results

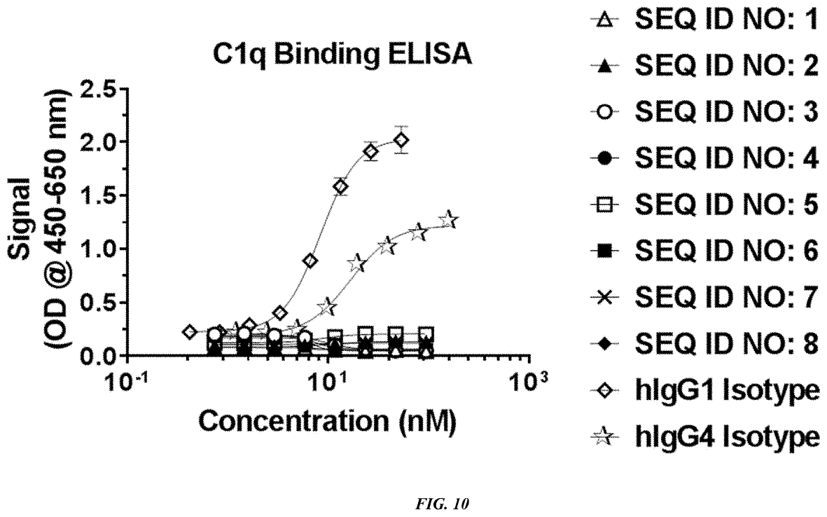

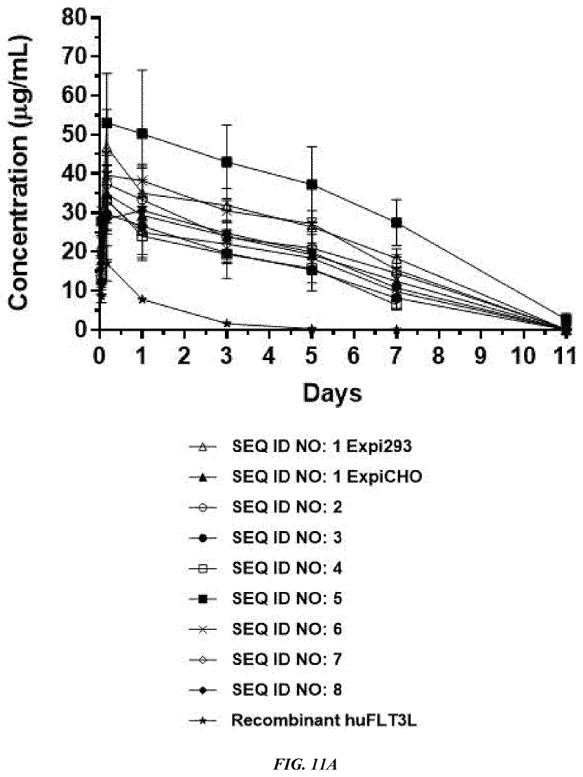

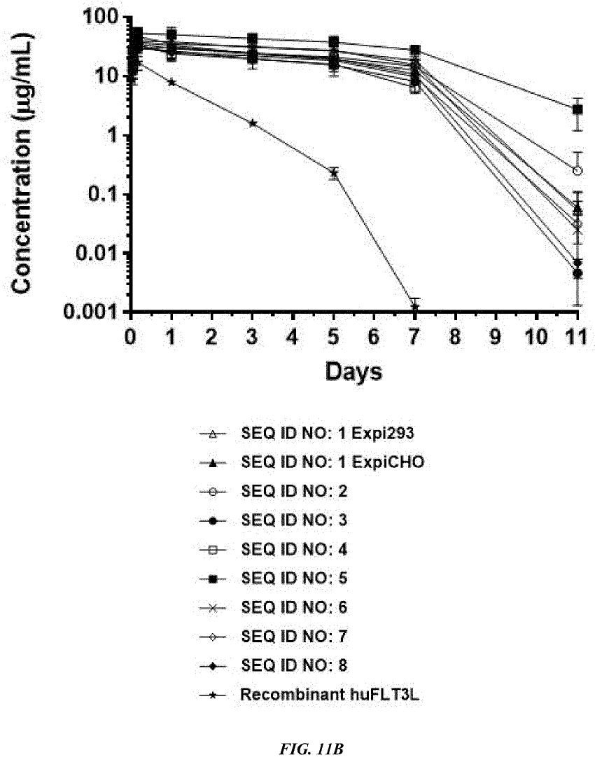

These data demonstrated that the eight human FLT3-ligand Fc fusion proteins with different Fc variants (SEQ ID NOs: 1-8) were devoid of C1q binding ability. Both the human IgG1 and IgG4 isotype controls demonstrated binding to C1q, with the IgG4 isotype showing reduced binding compared to the IgG1. The results are summarized in Table 10 and depicted in FIG. 10.

TABLE 10
EC50 Values for FLT3L-Fc Variants Binding to Human C1q

| FLT3L-Fc Variant | |
|---|---|
| SEQ ID NO: | EC50 (nM) |
| 1 | NA |
| 2 | NA |
| 3 | NA |
| 4 | NA |
| 5 | NA |
| 6 | NA |
| 7 | NA |
| 8 | NA |
| hIgG1 Isotype | 8.40 |
| hIgG4 Isotype | 16.69 |

### Example 11

### In Vivo Pharmacokinetics of FLT3L-Fc Variants in Mice

In this example, we compared the single dose pharmacokinetics of eight different human FLT3-ligand human Fc fusion proteins in C57Bl/6 mice.

### Methods

FLT3L-Fc Variants (SEQ ID NOs:1-8) were administered to male C57Bl/6 mice n=4/group (Covance, Wis.) at 5 mg/kg via a single intraperitoneal (IP) injection to characterize their basic pharmacokinetic (PK) profiles. Serial serum samples collected from mice were analyzed using U-PLEX FLT3L assay (Mesos Scale Discovery, MSD) according to the manufacturer's instructions. The calibration curve used the respective individual FLT3-ligand fusion proteins as reference standards in spiked mouse matrix fit to a 4-parameter logistic model with 1/Y2 weighting. Analyte concentrations were determined from the electrochemiluminescence signals back-fitted to the calibration curve. Serum concentration-time profiles were used to calculate the mean±SD serum PK parameters by non-compartmental PK analysis. Area under the curve (AUC0-7d) was determined through day 7 due to development of immunogenicity. Clearance (Cl/F) and half-life values reported in Table 11 are considered estimates due to the incomplete terminal extrapolation.

### Results

Pharmacokinetic analysis demonstrated that all eight Fc-fusions significantly enhanced the AUC exposure by approximately 5-12-fold in comparison to native human Flt3L, resulting in reduced FLT3-ligand clearance and prolonging the half-life (Table 11). We further observed a potential role of N-linked glycosylation on the pharmacokinetics because the aglycosylated FLT3L-Fc variant (SEQ ID NO: 5) had the highest AUC exposure. Additionally, we observed cell line dependent differences in PK evident by comparing data for samples corresponding to SEQ ID NO:1 produced in either Expi293 or ExpiCHO cells. The results are summarized in Table 11 and depicted in FIGS. 11A-11B.

TABLE 11
Single-Dose Pharmacokinetic Values for SEQ ID NOs: 1-8 in C57Bl/6 mice.

| | AUC_{0-7d} | Cl/F* | Cₘₐₓ | |
|---|---|---|---|---|
| SEQ ID NO | (μg*d/mL) | (mL/d/kg) | (μg/mL) | Half-life* (d) |
| 1-Expi293 | 209 ± 28.2 | 13.3 ± 2.26 | 46.8 ± 9.72 | 6.54 ± 0.653 |
| 1-ExpiCHO | 159 ± 29.6 | 20.9 ± 7.07 | 34.8 ± 7.45 | 5.15 ± 1.40 |
| 2 | 171 ± 45.7 | 18.9 ± 6.11 | 38.9 ± 8.13 | 5.34 ± 0.795 |
| 3 | 131 ± 9.60 | 28.9 ± 2.83 | 29.7 ± 4.00 | 3.64 ± 0.313 |
| 4 | 128 ± 37.4 | 33.3 ± 9.38 | 33.3 ± 8.80 | 3.34 ± 0.162 |
| 5 | 287 ± 71.4 | 8.72 ± 2.38 | 54.1 ± 13.4 | 8.02 ± 3.24 |
| 6 | 205 ± 8.77 | 15.9 ± 3.10 | 42.3 ± 1.57 | 5.10 ± 1.54 |
| 7 | 143 ± 26.1 | 25.1 ± 6.38 | 33.0 ± 7.63 | 4.63 ± 1.00 |
| 8 | 158 ± 52.3 | 24.4 ± 8.02 | 31.9 ± 11.4 | 4.16 ± 0.238 |
| Recombinant | 23.5 ± 3.84 | 215 ± 35.4 | 17.1 ± 4.51 | 0.785 ± 0.0441 |
| huFLT3L | | | | |

*CL/F and Half-life are estimates for Fc-fusions due to incomplete terminal extrapolation.

### Example 12

### Ability of FLT3L-Fc Variants to Promote Proliferation and Expansion of cDC1

In this example, we compared the ability of eight different human FLT3-ligand human Fc fusion proteins (SEQ ID NOs: 1-8) to induce proliferation of and expand conventional dendritic cell subtype 1 (cDC1) in C57Bl/6.

### Methods

Spleens were harvested from C57BL/6 mice from FIG. 11 at Day 11 post injection at 4° C. in HypoThermosol solution (BioLife Solutions). Spleens were then dissociated by using the gentleMACS Dissociator (Miltenyi Biotec) with heaters, following manufacturer's protocol. After enzymatic digestion, cell suspension was filtered through a 70 μm cell strainer. The remaining tube and strainer were rinsed 1× with 15-20 ml of RPMI and collected with the rest of the sample. Cells were centrifuged at 500 g for 5 min at room temperature. Supernatant was discarded and cells were washed 1× with PBS. Residual red blood cells were lysed by adding 2 ml of ACK lysis to each sample for 1-2 min at room temperature. FACS staining Buffer (BD Bioscience) was added to the samples to stop the ACK lysis activity. Cells were spun down and washed additionally with PBS. Samples were then stained with Live/Dead Fixable Aqua Dead Cell Stain Kit (ThermoFisher) at 1:750 dilution for 15 min at 4° C. Cells were washed 2× with FACS staining buffer, then Fc blocked for 30 min at 4° C. FACS antibodies (Biolegend) were directly added to the blocked samples and incubated at 4° C. for 30 min without spinning down or washing out the Fc block. Cells were washed 2×, resuspended in Staining Buffer, and analyzed by LSR Fortessa FACS analyzer. Raw data were analyzed by FlowJo X (BD Bioscience).

### Results

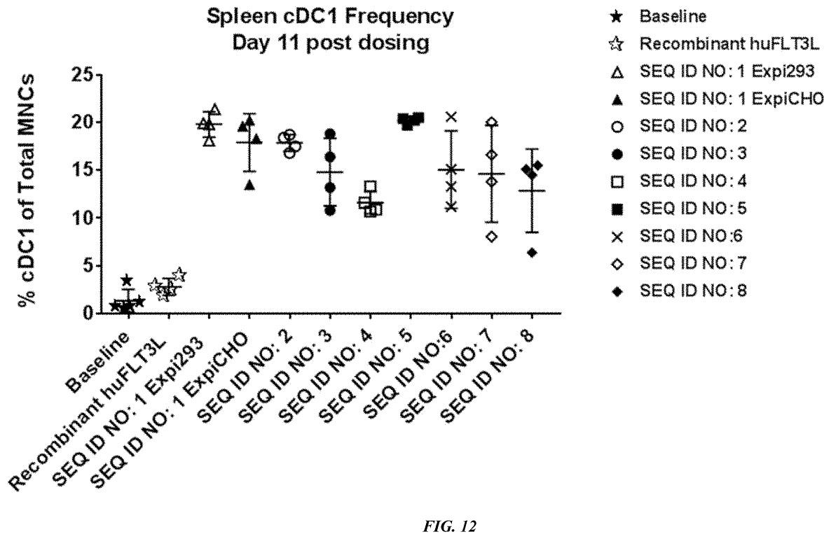

The data demonstrated that the ability of the eight human FLT3-ligand Fc fusion proteins with different Fc variants (SEQ ID NOs: 1-8) to expand splenic conventional dendritic cell subtype 1 (cDC1) in vivo at day 11 in mice was greater than that of the recombinant FLT3-ligand after a single dose administration at day 0. The results are summarized in Table 12 and depicted in FIG. 12.

TABLE 12
Average Frequency of Splenic cDC1 at day 11 in C57Bl/6 Mice
Injected with FLT3L-Fc Variants SEQ ID NO: 1-8 at Day 0

| FLT3L-Fc Variant | |
|---|---|
| SEQ ID NO: | % cDC1 in total MNCs |
| 1-Expi293 | 19.8 |
| 1-ExpiCHO | 17.9 |
| 2 | 17.85 |
| 3 | 14.8 |
| 4 | 11.62 |
| 5 | 20.2 |
| 6 | 15.05 |
| 7 | 14.62 |
| 8 | 12.87 |
| Recombinant huFLT3L | 2.8 |
| Baseline | 1.36 |

### Example 13

### In Vitro Potency of FLT3L-Fc Variants in Cell Proliferation Assay

In this example, we compared the in vitro potency of four different human FLT3-ligand human Fc fusion proteins: human FLT3-ligand human hingeless IgG1 fusion protein (SEQ ID NO:1), human FLT3-ligand (Δ5 amino acid) human IgG4 (S228P/F234A/L235A) fusion protein (SEQ ID NO:6), human FLT3-ligand human hingeless IgG1 (M252Y/S254T/T256E) fusion protein (SEQ ID NO:9), or human FLT3-ligand (Δ5 amino acid) human IgG4 (S228P/F234A/L235A/M252Y/S254T/T256E) fusion protein (SEQ ID NO:14). To evaluate the in vitro potency, we employed an AML5 cell proliferation assay. The methods are as described above in Example 5.

### Results

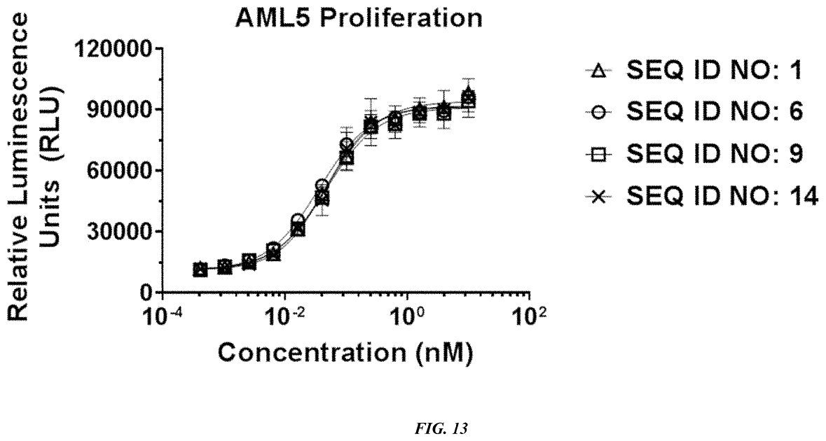

These data demonstrated that the potency of the four human FLT3-ligand Fc fusion proteins with different Fc variants (SEQ ID NOs: 1, 6, 9 and 14) in inducing FLT3-dependent proliferation in AML5 cells was similar, with EC50 values ranging between 0.037-0.050 nM. The results are summarized in Table 13 and depicted in FIG. 13.

TABLE 13
EC50 values for FLT3L-Fc Variant SEQ ID NOs: 1, 6, 9 and 14
in AML5 Proliferation Assay

| FLT3L-Fc Variant | |
|---|---|
| SEQ ID NO: | EC50 (nM) |
| 1 | 0.050 |
| 6 | 0.048 |
| 9 | 0.037 |
| 14 | 0.046 |

### Example 14

### In Vitro Potency of FLT3L-Fc Variants in cDC1 Differentiation Assay

In this example, we compared the in vitro potency of four different human FLT3-ligand human Fc fusion proteins to differentiate human bone marrow CD34+ stem cells into conventional dendritic cell subtype 1 (cDC1).

### Methods

96-well flat-bottom tissue culture plates (Falcon, 353072) were coated with recombinant DLL1 (R&D Systems, 1818-DL-050) as follows. DLL1 was reconstituted in PBS to create a stock solution of 500 μg/ml. The stock solution was diluted in DPBS (Corning, 21-030-CV) to a final working concentration of 5 μg/ml, and 100 μl of this was plated into each well. Plates were sealed and placed on a flat surface at 4° C. overnight.

Bone marrow CD34+ stem cells from 13 healthy donors were thawed in a 37° C. water bath and transferred into complete media (Alpha-Mem (Gibco, 12561056), 10% heat-inactivated FCS, lx Pen/Strep). To recover the cells, 20,000 cells per well were plated into a 96-well round bottom tissue culture plate.

The next day, the DLL1 coated plates were washed 3× with DBPS, then 10,000 recovered cells per well were cultured with 20 ng/ml human GM-CSF, 20 ng/ml human SCF, 2.5 ng/ml human IL-4 and various test articles. On day 6, half the media was removed and fresh cytokines and compounds were added to the cells. On day 14, cells were collected. Staining antibodies were then added to the cells and incubated for 30 min at 4° C. Cells were then washed twice with FACS staining buffer and analyzed LSR Fortessa FACS analyzer (BD Bioscience). Raw data were analyzed by FlowJo X (BD Bioscience).

### Results

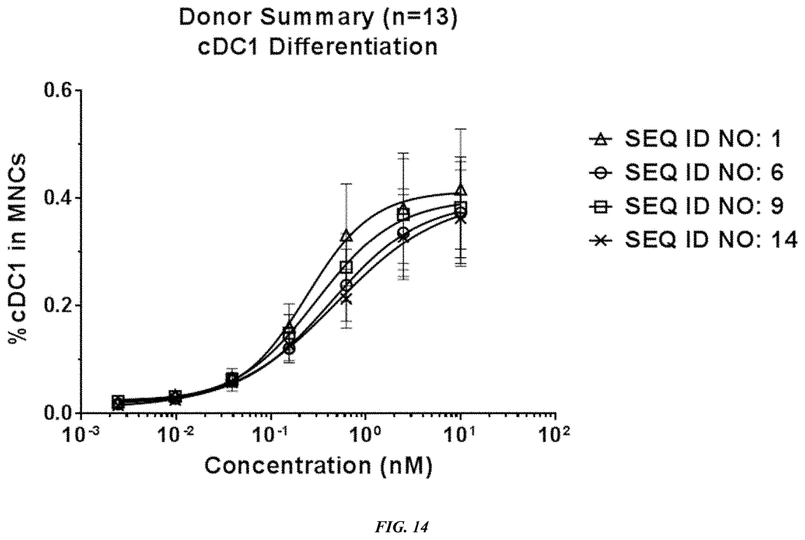

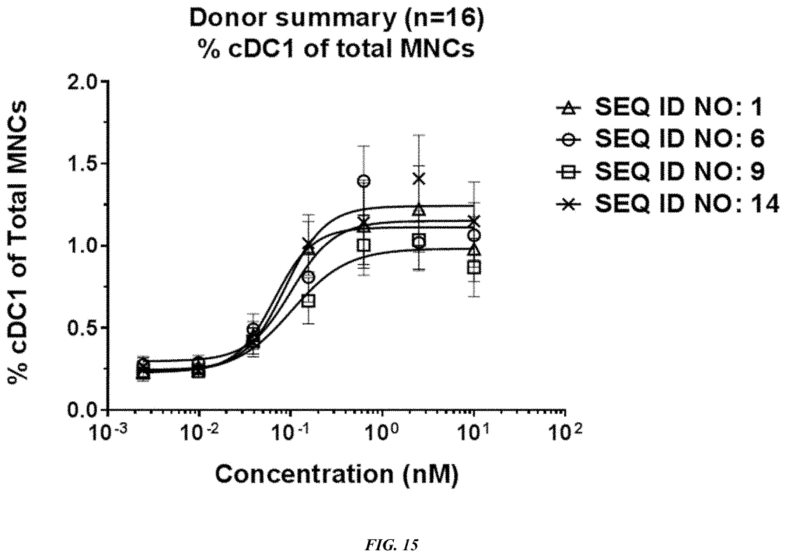

These data demonstrated that the potency of the four human FLT3-ligand Fc fusion proteins with different Fc variants (SEQ ID NOs: 1, 6, 9 and 14) in inducing cDC1 differentiation in vitro from primary human CD34+ bone marrow stem cells was similar, with EC50 values ranging between 0.788-1.252 nM. The results are summarized in Table 14 and depicted in FIG. 14.

TABLE 14
EC50 values for differentiation of human CD34+ stem cells into cDC1 by proteins
corresponding to FLT3L-Fc Variant SEQ ID NOs: 1, 6, 9 and 14

| FLT3L-Fc | |
|---|---|
| Variant | |
| SEQ ID NO: | EC50 (nM) |
| 1 | 1.252 |
| 6 | 1.031 |
| 9 | 0.915 |
| 14 | 0.788 |

### Example 15

### In Vitro Potency of FLT3L-Fc Variants in Promoting cDC1 Survival

In this example, we compared the in vitro potency of four different human FLT3-ligand human Fc fusion proteins to enhance the survival of human PBMC-derived conventional dendritic cell subtype 1 (cDC1).

### Methods

Sixteen fresh human healthy donor PBMCs were obtained from PPA Research Group Inc. Pan-DCs were isolated from the PBMCs following the manufacturer's protocol for EasySep Human Pan-DC Pre-Enrichment Kit (Stemcell Technologies, Inc, 19251). Pan-DCs were then stained with eBioscience Cell Proliferation Dye efluor 450 (Invitrogen, 65-0842-85) for 8 min in a 37° C. water bath. After staining with the cell proliferation dye, cells were washed 2× and resuspended with complete RPMI. 100,000 cells per well were plated into a 96-well round bottom tissue culture plate and titrations of the compounds were added to the cells for a final volume of 200 ul per well. Plates were sealed with a breathable plate sealer (Breathe Easy Sealing Membrane, Millipore Sigma, Z380059-1Pak) and then incubated for 4 days at 37° C. After incubation, cells were washed with FACS staining buffer and Fc blocked (Human TruStain FcX Biolegend, 422302) for at least 10 min at 4° C. Staining antibodies were then directly added to the cells and incubated for 30 min at 4° C. Cells were then washed twice with FACS staining buffer and analyzed LSR Fortessa FACS analyzer (BD Bioscience). Raw data were analyzed by FlowJo X (BD Bioscience).

### Results

The data demonstrated that the potency of the 4 human FLT3-ligand Fc fusion proteins with different Fc variants (SEQ ID NOs: 1, 6, 9 and 14) in enhancing primary human cDC1 survival in vitro was similar, with EC50 values ranging between 0.067-0.102 nM. The results are summarized in Table 15 and depicted in FIG. 15.

TABLE 15
EC50 values for promoting cDC1 survival by proteins
corresponding to FLT3L-Fc Variant SEQ ID NOs: 1, 6, 9 and 14

| FLT3L-Fc | |
|---|---|
| Variant | |
| SEQ ID NO: | EC50 (nM) |
| 1 | 0.067 |
| 6 | 0.102 |
| 9 | 0.102 |
| 14 | 0.087 |

### Example 16

### In Vitro Binding of FLT3L-Fc Variants to FLT3

In this example, we compared the in vitro binding of four different human FLT3-ligand human Fc fusion proteins to human recombinant FLT3 by ELISA. The methods are as described above in Example 6.

### Results

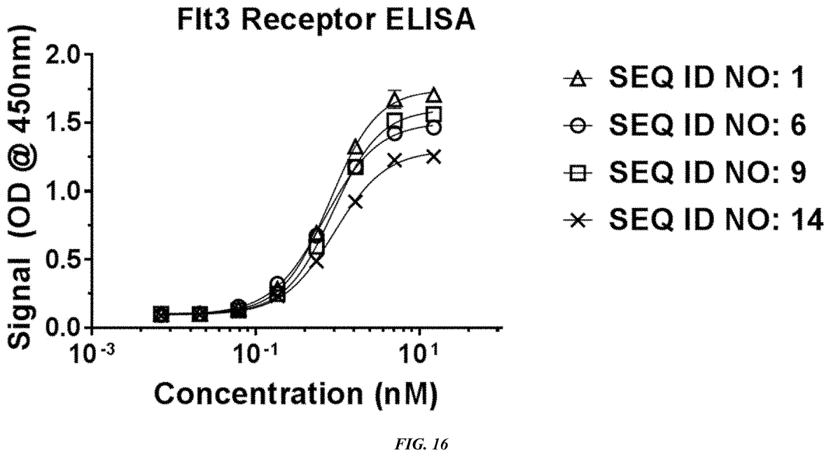

These data demonstrated that binding to human FLT3 receptor was similar among the four human FLT3-ligand Fc fusion proteins with different Fc variants (SEQ ID NOs: 1, 6, 9 and 14), with EC50 values ranged between 0.70 to 0.92 nM. The results are summarized in Table 16 and depicted in FIG. 16.

TABLE 16
EC50 values for FLT3L-Fc Variant SEQ ID NOs: 1, 6, 9 and 14
Binding to Human Recombinant FLT3

| FLT3L-Fc | |
|---|---|
| Variant | EC50 |
| SEQ ID NO: | (nM) |
| 1 | 0.81 |
| 6 | 0.70 |
| 9 | 0.88 |
| 14 | 0.92 |

### Example 17

### In Vitro Binding of FLT3L-Fc Variants to FcRn

In this example, we compared the in vitro binding of four different human FLT3-ligand human Fc fusion proteins to human recombinant FcRn by ELISA. The methods are as described above in Example 7.

### Results

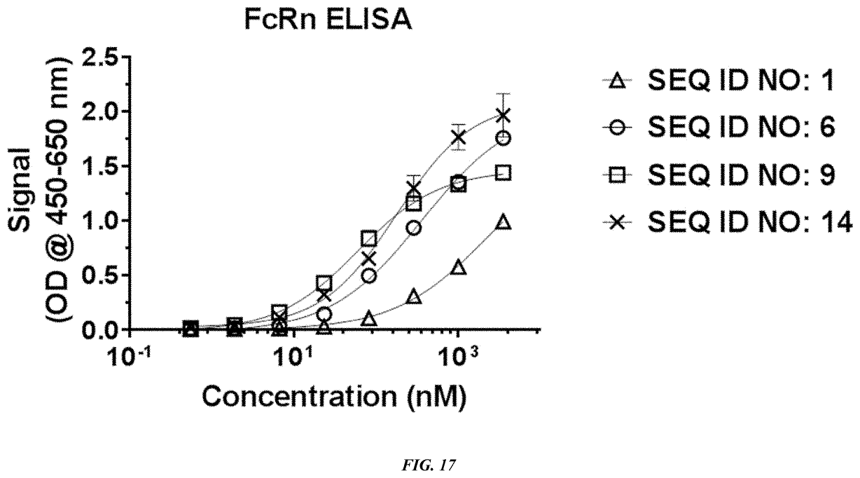

The data demonstrated that M252Y/S254T/T256E mutations in the human IgG Fc region leads to improved FcRn binding for SEQ ID NOs: 9 and 14, compared to their counterparts without these mutations (SEQ ID NOs: 1 and 6). A 38-fold increase in FcRn binding was observed for Flt3L-Fc in the hingeless IgG1 format (SEQ ID NOs: 1 and 9), compared to a 2-fold increase in the IgG4 format (SEQ ID NOs: 6 and 14). The results are summarized in Table 17 and depicted in FIG. 17.

TABLE 17
EC50 Values for FLT3L-Fc Variant SEQ ID NOs: 1, 6, 9 and 14
Binding to Human Recombinant FcRn

| FLT3L-Fc | |
|---|---|
| Variant | |
| SEQ ID NO: | EC50 (nM) |
| 1 | 2332 |
| 6 | 365.00 |
| 9 | 61 |
| 14 | 178 |

### Example 18

### Ability of FLT3L-Fc Variants to Compete for Binding to Human FcγRI

In this example, we compared the in vitro ability of four different human FLT3-ligand human Fc fusion proteins to compete for binding of human recombinant FcγRI with a human IgG molecule. To evaluate the ability to compete for binding to FcγRI, we employed an amplified luminescent proximity homogeneous assay (AlphaScreen® by Perkin Elmer). The methods are as described above in Example 8.

### Results

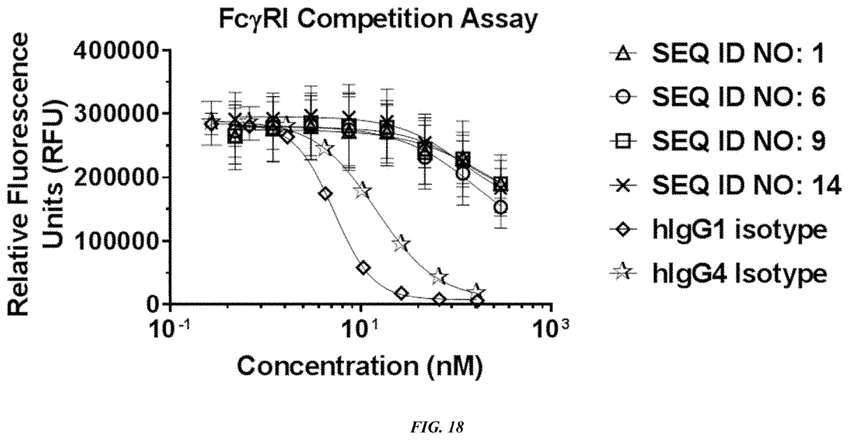

These data demonstrated that none of the 4 human FLT3-ligand Fc fusion proteins with different Fc variants (SEQ ID NOs: 1, 6, 9 and 14) could fully compete with human IgG for binding to FcγRI at the highest concentrations tested. Both the human IgG1 and IgG4 isotype antibody controls demonstrated complete dose-response curves, with the IgG4 isotype showing reduced competition compared to the IgG1. The results are summarized in Table 18 and depicted in FIG. 18.

TABLE 18
EC50 values for the ability of FLT3L-F Variant SEQ ID NOs: 1, 6, 9 and 14 to compete
with a human IgG molecule for binding to human recombinant FcγRI

| FLT3L-Fc | |
|---|---|
| Variant | |
| SEQ ID NO: | EC50 (nM) |
| 1 | NA |
| 6 | NA |
| 9 | NA |
| 14 | NA |
| hIgG1 Isotype | 5.25 |
| hIgG4 Isotype | 14.54 |

### Example 19

### Ability of FLT3L-Fc Variants to Compete for Binding to Human FcγRIIIa

In this example, we compared the in vitro ability of four different human FLT3-ligand human Fc fusion proteins to compete for binding of human recombinant FcγRIIIa (Val176 variant) with a human IgG molecule. To evaluate the ability to compete for binding to FcγRIIIa, we employed an amplified luminescent proximity homogeneous assay (AlphaScreen® by Perkin Elmer). The methods are as described above in Example 9.

### Results

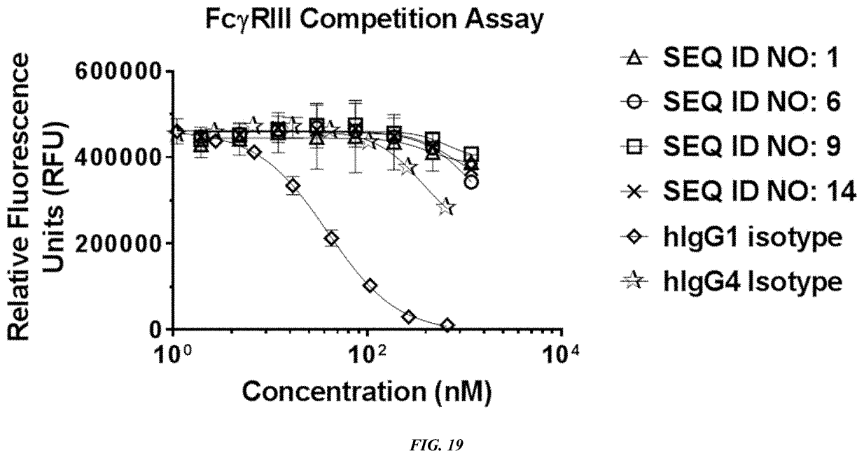

The results demonstrated that none of the 4 human FLT3-ligand Fc fusion proteins with different Fc variants (SEQ ID NOs: 1, 6, 9 and 14) could fully compete with human IgG for binding to FcγRIIIa (Val176 variant). Only human IgG1 isotype antibody control demonstrated a complete dose-response curve. The results are summarized in Table 19 and depicted in FIG. 19.

TABLE 19
EC50 values for the ability of FLT3L-F Variant SEQ ID NOs: 1, 6, 9 and 14 to compete with a
human IgG for binding to human recombinant FcγRIIIa (V-variant)

| FLT3L-Fc Variant | |
|---|---|
| SEQ ID NO: | EC50 (nM) |
| 1 | NA |
| 6 | NA |
| 9 | NA |
| 14 | NA |
| hIgG1 Isotype | 38.80 |
| hIgG4 Isotype | 368.30 |

### Example 20

### In Vitro Binding of FLT3L-Fc Variants to Human C1q

In this example, we compared the in vitro binding of four different human FLT3-ligand human Fc fusion proteins to human recombinant C1q. To evaluate the in vitro binding to C1q, we employed an ELISA. The methods are as described above in Example 10.

### Results

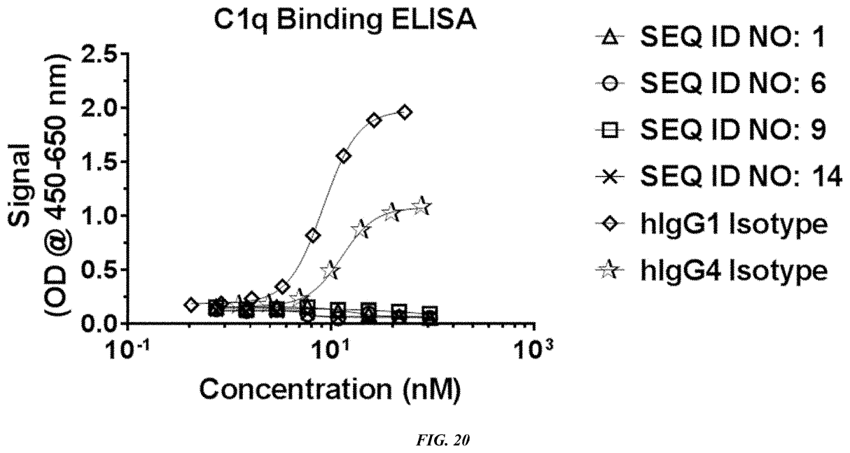

These data demonstrated that the four human FLT3-ligand Fc fusion proteins with different Fc variants (SEQ ID NOs: 1, 6, 9 and 14) are devoid of C1q binding ability. Both the human IgG1 and IgG4 isotype controls demonstrated binding to C1q, with the IgG4 isotype showing reduced binding compared to the IgG1. The results are summarized in Table 20 and depicted in FIG. 20.

TABLE 20
EC50 values for FLT3L-Fc Variants SEQ ID NOs: 1, 6, 9 and 14
Binding to Human Recombinant C1q

| FLT3L-Fc | |
|---|---|
| Variant | |
| SEQ ID NO: | EC50 (nM) |
| 1 | NA |
| 6 | NA |
| 9 | NA |
| 14 | NA |
| hIgG1 Isotype | 8.44 |
| hIgG4 Isotype | 13.71 |

### Example 21

### Single Dose Pharmacokinetics of FLT3L-Fc Variants in Cynomolgus Macaque

In this example, we compared the single dose pharmacokinetics of four different human FLT3-ligand human Fc fusion proteins in Cynomolgus macaque.

### Methods

Four FLT3L-Fc variants (SEQ ID NOs: 1, 6, 9, and 14) were administered to cynomologus macaques n=3/group (Covance, Tex.) at 0.5 mg/kg via a single intravenous (IV) and subcutaneous (SC) injection to characterize their basic PK profiles. Serial serum samples collected from macaques were analyzed using U-PLEX FLT3L assay (Meso Scale Discovery, MSD) according to the manufacturer's instructions. The calibration curve used the respective individual FLT3-ligand fusion proteins as reference standards in spiked macaque matrix fit to a 4-parameter logistic model with 1/Y2 weighting. Analyte concentrations were determined from the electrochemiluminescence signals back-fitted to the calibration curve. Serum concentration-time profiles were used to calculate the mean±SD serum PK parameters by non-compartmental PK analysis.

### Results

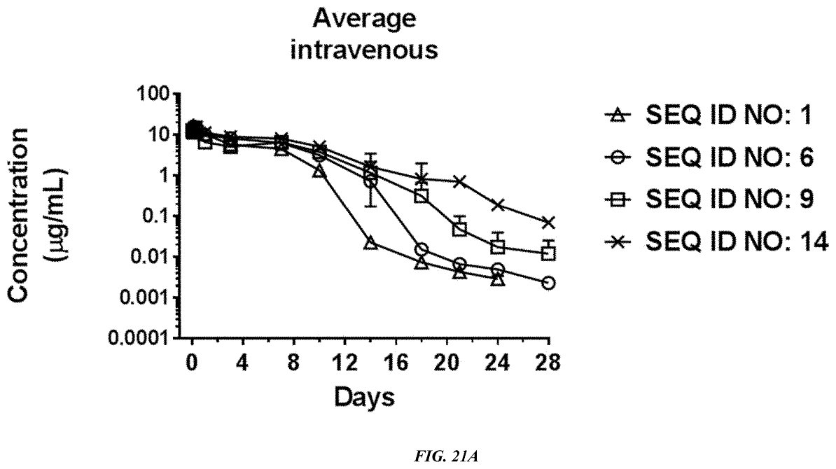

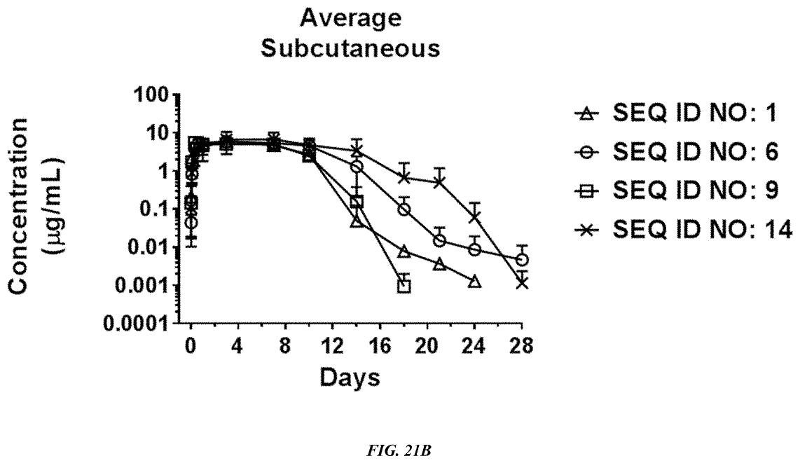

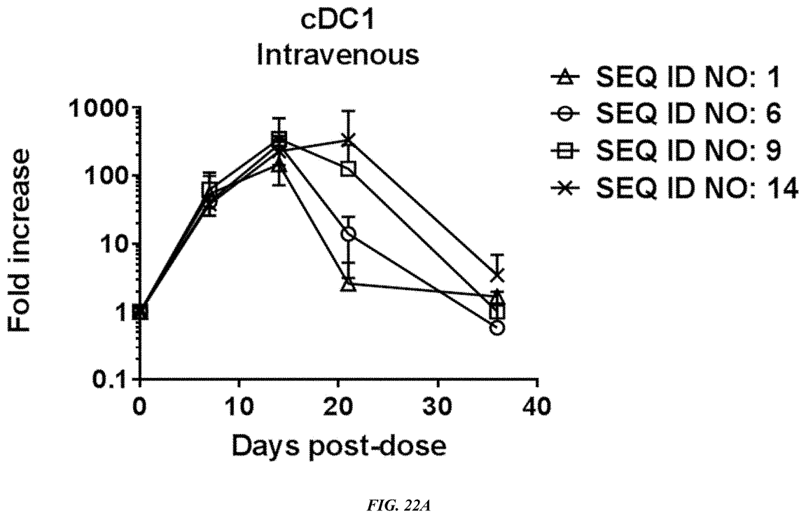

Pharmacokinetic (PK) analysis demonstrated that all 4 Fc-fusions had IgG-like pharmacokinetics with C1 values range from 4.71-7.74 mL/d/kg (Table 21) following IV administration in cynomologus macaques. Similar pharmacokinetics were observed following subcutaneous administration with bioavailability ranging from 66.8-91.4%. SEQ ID NOs: 9 and 14 containing the M252Y/S254T/T256E modifications had reduced clearance relative to unmodified sequence, consistent with improved FcRn binding. The results are summarized in Table 21 and depicted in FIGS. 21A-21B.

TABLE 21
Single dose PK values for FLT3L-F Variants SEQ ID NOs: 1, 6, 9
and 14 in Cynomolgus macaques

| FLT3L-Fc | | | | |
|---|---|---|---|---|
| SEQ | Intravenous | | Subcutaneous | |
| ID NO: | AUC₀₋ₗₐₛₜ | | AUC₀₋ₗₐₛₜ | |
| Variant | (μg*d/mL) | Cl (mL/d/kg) | (μg*d/mL) | Cl/F (mL/d/kg) |
| 1 | 59.3 ± 5.29 | 7.74 ± 1.35 | 54.2 ± 5.04 | 9.29 ± 0.91 |
| 6 | 82.4 ± 15.6 | 6.23 ± 1.31 | 66.2 ± 20.1 | 7.98 ± 2.10 |
| 9 | 73.8 ± 12.2 | 6.89 ± 1.08 | 49.3 ± 3.36 | 9.16 ± 1.01 |
| 14 | 107 ± 11.6 | 4.71 ± 0.502 | 79.4 ± 30.4 | 7.04 ± 3.27 |

### Example 22

### Ability of FLT3L-Fc Variants to Promote cDC1 Proliferation in Cynomolgus Macaque

In this example, we compared the ability of four different FLT3L-Fc variants (SEQ ID NOs: 1, 6, 9 and 14) to induce proliferation and expansion of conventional dendritic cell subtype 1 (cDC1) in Cynomolgus macaque.

### Methods

Whole blood samples from cynomolgus macaques administered one of SEQ ID NO: 1, 6, 9, or 14 at day 0 were drawn into Sodium Heparin blood collection tubes at indicated time points. One hundred microliters of each sample were transferred to FACS tubes containing FACS antibodies and Fc blocker. Cells were incubated at room temperature for 20 min, then washed twice with 1×DPBS-CMF. Residual red blood cells were then lysed for 8-12 minutes in the dark at room temperature with 1 mL 1×FACS Lyse (BD Biosciences). After incubation, the samples were centrifuged and washed once by adding 1×DPBS-CMF (1 mL). Samples were then resuspended in 125 μL 1×DPBS-CMF and 100 μL CountBright Beads for acquisition on the Canto FACS analyzer (BD Biosciences). Raw data were analyzed by FlowJo X (BD Bioscience).

### Results

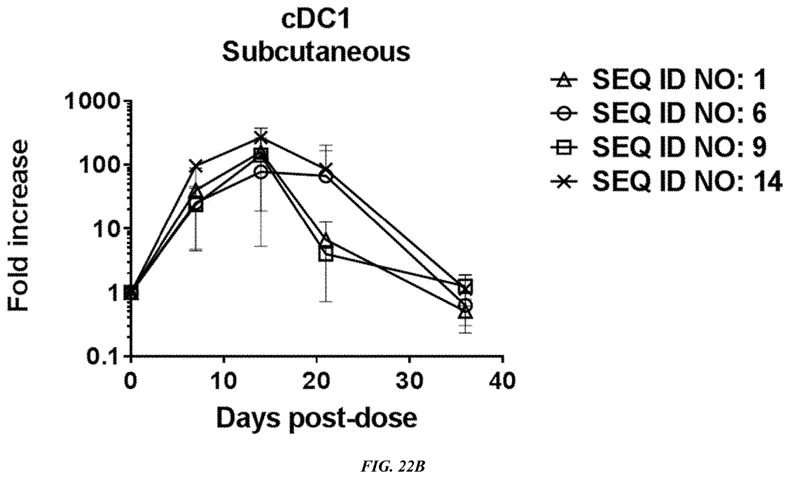

These data demonstrated a similar ability of the four human FLT3-ligand Fc fusion proteins with different Fc variants (SEQ ID: 1, 6, 9, and 14) to expand conventional dendritic cell subtype 1 (cDC1) in peripheral blood of Cynomolgus macaque from day 0 to day 14 after single-dose intravenous or subcutaneous administration. The cDC1 number of each group gradually declined to baseline levels between days 14 to 38 after test article administration, at a rate which paralleled the corresponding decline in serum levels of FLT3-ligand Fc fusion proteins as shown in FIG. 21. These results are depicted in FIGS. 22A-22B.

### Example 23

### Anti-Tumor Activity of Murine Surrogate FLT3L-Fc Fusion Protein

In this example, we demonstrated the anti-tumor activity of a murine surrogate FLT3L-Fc fusion protein (SEQ ID NO:20) in a mouse tumor model.

### Methods

Eight-week old C57Bl/6 mice (Jackson Laboratory) were inoculated with 2.5×10⁵ MC38 cells subcutaneously. Mice were then randomized when tumor volume reached 45-55 mm³ on day 0 and dosed intraperitoneally with SEQ ID NO: 20 or an Fc-silent (N297A) mouse IgG2a isotype control at indicated concentration within the same day. Tumors were measured 3 times a week using calipers. Tumor volumes were calculated using the following equation: (longest diameter*shortest diameter²)/2.

### Results

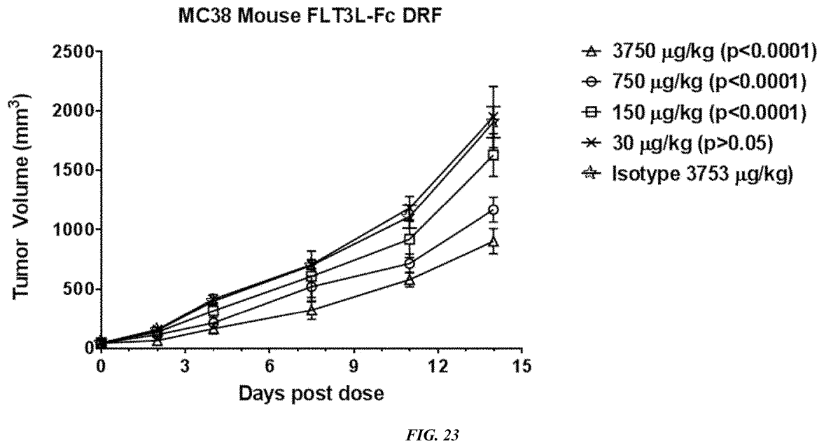

The data demonstrated a dose-dependent tumor growth inhibition of MC38 tumors in C57BL/6 mice after a single-dose intraperitoneal administration by the mouse FLT3-ligand Fc-fusion protein (SEQ ID NO:20) at day 0. Groups dosed with 150 μg/kg, 750 μg/kg and 3750 μg/kg of SEQ ID NO:20 show significantly slower tumor growth rates compared to the isotype control group. The results are summarized in Table 22 and depicted in FIG. 23.

TABLE 22
Tumor Growth Inhibition (TGI) at Day 14 Post-Dose for SEQ ID
NO: 20 Relative to Isotype Control in MC38 Mouse Tumor Model

| Dose | TGI (%) | p-value |
|---|---|---|
| 3750 μg/kg | 52.67 | <0.0001 |
| 750 μg/kg | 38.68 | <0.0001 |
| 150 μg/kg | 14.51 | <0.0035 |
| 30 μg/kg | −2.35; | not significant |

### Example 24

### Intratumoral and Peripheral Expansion of cDC1 in Tumor Bearing Mice

In this example, we demonstrated the intratumoral and peripheral expansion of conventional dendritic cell subtype 1 (cDC1) in tumor bearing mice by FLT3L-Fc fusion protein using a murine surrogate (SEQ ID NO:20).

### Methods

Tumors and spleens were harvested at Day 7 post administration and shipped 0/N at 4° C. in HypoThermosol solution (BioLife Solutions) from the CROs. Tumors and spleens were then dissociated using the gentleMACS Dissociator (Miltenyi Biotec) with heaters, following manufacturer's protocol. After enzymatic digestion, cell suspension was filtered through a 70 μm cell strainer. The remaining tube and strainer were rinsed 1× with 15-20 ml of RPMI and collected with the rest of the sample. Cells were centrifuged at 500×g for 5 min at room temperature. Supernatant was discarded and cells were washed 1× with PBS. Residual red blood cells from spleens were lysed by adding 2 ml of ACK lysis buffer to each sample for 1-2 min at room temperature. FACS staining Buffer (BD Bioscience) was added to the samples to stop the ACK lysis activity. Cells were spun down and washed additionally with PBS. Samples were then stained with Live/Dead Fixable Aqua Dead Cell Stain Kit (ThermoFisher) at 1:750 dilution for 15 min at 4° C. Ten microliters of cells from each sample were taken for count by 123count eBeads (eBiosciences) following manufacturer's protocol. Cells were washed 2× with FACS staining buffer, then Fc blocked for 30 min at 4° C. FACS antibodies (Biolegend) were directly added to the blocked samples and incubated at 4° C. for 30 min without spinning down or washing out the Fc block. Cells were washed 2×, resuspended in Staining Buffer, and analyzed by LSR Fortessa FACS analyzer. Raw data were analyzed by FlowJo X (BD Bioscience).

### Results

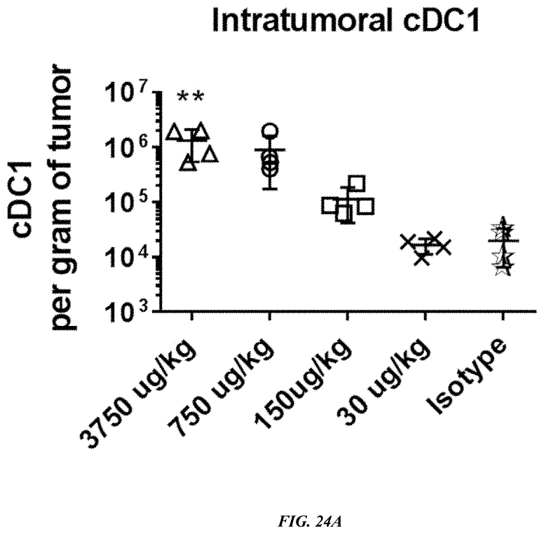

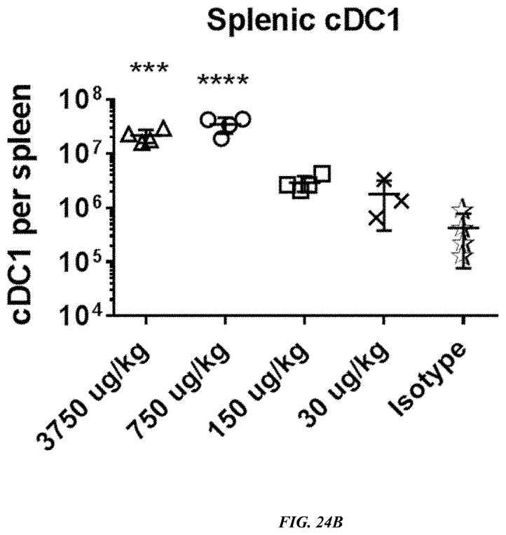

The results demonstrated a dose-dependent increase of conventional dendritic cell subtype 1 (cDC1) number in tumors (FIG. 24A) or in spleens (FIG. 24B) in the MC38 tumor model after a single-dose intraperitoneal administration by the mouse surrogate FLT3-ligand Fc-fusion protein (SEQ ID NO:20) at day 0. Groups dosed 750 □g/kg and 3750 μg/kg of SEQ ID NO:20 showed a significant increase of cDC1 number in tumors compared to the isotype group, while groups dosed 750 μg/kg and 3750 μg/kg of SEQ ID NO:20 show significant increase of cDC1 number in spleens compared to the isotype group. Similar increase of cDC1 number in tumors and spleens was observed in groups dosed with 750 μg/kg and 3750 μg/kg of SEQ ID NO:20. The results are summarized in Table 23 and depicted in FIG. 24.

TABLE 23
cDC1 Fold-Change in Tumor and Spleen at day 7 Post-Dose for SEQ
ID NO: 20 Relative to Isotype Control in MC38 Mouse Tumor Model

| | cDC1 fold change in | cDC1 fold change in |
|---|---|---|
| Dose | tumor | spleen |
| 3750 μg/kg | 65.57 | 50.7 |
| 750 μg/kg | 44.21 | 81.76 |
| 150 μg/kg | 4.72 | 6.84 |
| 30 μg/kg | −0.17 | 4.18 |

### Example 25

### Sialic Acid Content Evaluation of Eight FLT3L-Fc Variants

In this example, we determined the total sialic acid content of eight Flt3L-Fc constructs. To evaluate sialic acid content, we performed chemical release of sialic acids followed by fluorescence labeling and reversed-phase separation with fluorescence detection.

### Methods

Proteins from Flt3L-Fc constructs SEQ ID NOs: 1-8 were diluted to 10 or 50 μg/mL in water. Sialic acids were released by mild acid hydrolysis with acetic acid, separated from the protein by filtration, and fluorescently labeled with 1,2-diamino-4,5-methylenedioxybenzene dihydrochloride (DMB) by a reductive amination reaction. The labeled sialic acid products were then separated by reversed-phase chromatography using a C18 column with fluorescence detection at 373 nm (excitation) and 448 nm (emission). The concentrations of N-acetylneuraminic acid (NANA) and N-Glycolylneuraminic acid (NGNA) present were determined from an identically labeled 6-point standard curve and expressed as a mole to mole ratio of sialic acid to protein content.

### Results

The results are summarized in Table 24.

TABLE 24
Sialic Acid Content of SEQ ID NO. 1-8

| FLT3L-Fc | | |
|---|---|---|
| Variant | mol/mol | mol/mol |
| SEQ ID NO: | NANA | NGNA |
| 1 | 16.5 | ND* |
| 2 | 13.7 | ND |
| 3 | 15.2 | ND |
| 4 | 17.4 | ND |
| 5 | 14.3 | ND |
| 6 | 15.4 | ND |
| 7 | 8.1 | ND |
| 8 | 13.0 | ND |

*ND = not detected

The data obtained for SEQ ID NOs: 1-8 showed similar amounts of N-acetylneuraminic acid (NANA) in each sample, at an average of 15 mol/mol. One exception was noted, in which SEQ ID NO: 7 contained only 8 mol/mol sialic acid. A subtle trend was also observed in which sialic acid content decreased in constructs containing C-terminal truncations of the Flt3L portion of the fusion proteins (constructs SEQ ID NO: 2 and 7 relative to SEQ ID NO:1; constructs SEQ ID NO: 6 and 8 relative to SEQ ID NO: 4), indicating this region contained the majority of sialic acid. Furthering this idea was the observation that N-glycan removal from the ligand domain did not result in a major loss of sialic acid content, shown by comparison of SEQ ID NO: 5 and SEQ ID NO: 1.

Total sialic acid content was shown to have a positive correlation to PK in mAbs and Fc-fusion proteins, consistent with the reporting of, e.g., Li, et al., J Pharm Sci (2015) 104:1866-1884; and Liu, et al., Protein Cell. (2018) 9(1):15-32.

### Example 26

### Sialic Acid Content Evaluation of Four FLT3L-Fc Variants

In this example, we determined the sialic acid content on the two FLT3L N-glycans at positions 100 and 123 of SEQ ID NOs: 1, 6, 9, and 14 (“Asn100” and “Asn123,” respectively).

### Methods

Protein digest followed by reversed-phase separation with detection by mass spectrometry was performed.

Following denaturation, reduction and cysteine carboxymethylation, proteins from SEQ ID NOs: 1, 6, 9, and 14 were digested for 6 hours at 37° C. using a 1:10 enzyme:substrate (w:w) ratio of Lys-C and Glu-C Mix. Digestion was then quenched by adding trifluoroacetic acid to 0.1% final concentration. The resulting Lys-C/Glu-C peptides were separated by reverse phase gradient UPLC on a C18 column. The separation of the peptides was monitored at 214 nm prior to elution into the inlet of a Thermo Scientific QE HF Orbitrap mass spectrometer operating in positive, data-dependent acquisition mode. N-glycan peptide assignments were based on matching the observed masses of the intact peptides in the LC/MS analysis to the masses predicted based on a theoretical Lys-C and Glu-C digest of SEQ ID NOs: 1, 6, 9, and 14.

### Results

The results are presented in Table 25.

TABLE 25
Sialylalation Analysis of FLT3L-Fc
Variants SEQ ID NOs: 1, 6, 9, and 14

| | SEQ ID | | SEQ ID | | SEQ ID | | SEQ ID | |
|---|---|---|---|---|---|---|---|---|
| | NO. 1 | | NO. 6 | | NO. 9 | | NO. 14 | |
| | Sialylated | Occu- | Sialylated | Occu- | Sialylated | Occu- | Sialylated | Occu- |
| | Peptides¹ | pancy² | Peptides¹ | pancy² | Peptides¹ | pancy² | Peptides¹ | pancy² |
| | (%) | (%) | (%) | (%) | (%) | (%) | (%) | (%) |
| Asn | 43 | 66 | 43 | 64 | 33 | 61 | 46 | 63 |
| 100 | | | | | | | | |
| Asn | 63 | 70 | 57 | 68 | 39 | 63 | 57 | 67 |
| 123 | | | | | | | | |

| | Relative abundance of sialylated | | | |
|---|---|---|---|---|
| | FLT3L N-glycans corrected by occupancy (%) | | | |
| Asn | 65 | 68 | 54 | 72 |
| 100 | | | | |
| Asn | 90 | 84 | 62 | 94 |
| 123 | | | | |
| | Average across the two sites (%) | | | |
| | 77 | 76 | 58 | 83 |

¹FLT3L N-glycans with one or more sialic acid unit. Percentage determined including asialylated peptides
²N-glycan occupancy at the two sites was determined by peptide mapping

The molecular masses of the N-glycosylated peptides were consistent with the predicted masses based on the amino acid sequences of the FTL3L-Fc constructs. FTL3L-Fc SEQ ID NOs 1, 6, and 14 contained a consistent level of species with one or more sialic acid unit (43-63%, Table 25). FTL3L-Fc SEQ ID NO: 9 showed a decrease of approximately 10% at Asn 100 and 24% at Asn 123 (Table 25). Ligand N-glycans site occupancy for SEQ ID NOs: 1, 6, 9 and 14 was consistent across all four molecules tested and ranged from 61% to 70% across the two sites (Table 25).

The percent of ligand N-glycans species containing at least one sialic acid unit were corrected by the percent occupancy at each site and averaged across the two sites to allow a direct comparison across the constructs (Table 25). After correction and averaging, FTL3L-Fc SEQ ID NOs: 1, 6, and 14 showed a level of overall ligand sialylated N-glycans ranging from 76 to 83%; while FTL3L-Fc SEQ ID NO: 9 showed a lower overall level at 58%.

### Example 27

### Conformational Stability of FLT3L-Fc Variants

In this example, we evaluated the conformational stability of FLT3L-Fc variants SEQ ID NOs: 1 to 9 and SEQ ID NO: 14.

Each FLT3L-Fc construct was prepared in a matrix of 20 mM sodium phosphate, 9% sucrose, 0.02% PS80 pH 6.5 and loaded into a capillary. Intrinsic fluorescence was measured by a Nano differential scanning fluorimetry (NanoTemper) instrument as the samples were heated from 25-95° C. The ratio of fluorescence signal at 350/330 nm was plotted versus temperature to compare melting profiles of each sample. Duplicate measurements were performed for each construct.

The data obtained for FLT3L-Fc variants SEQ ID NOs: 1 to 8 show similar conformational stability, with onset temperatures (Tₒₙ) greater than 55° C. for each FLT3L-Fc variant. FLT3L-Fc SEQ ID NO 3 has a lower Tₘₗ compared to the three other IgG4 FLT3L-Fc constructs, indicating that the L235E mutation is slightly destabilizing resulting in lower conformational stability. Results of the IgG1 FLT3L-Fc constructs also show that FLT3L-Fc SEQ ID NO 5 has a decreased Tₒₙ and Tₘₗ compared with the three other IgG1 constructs (Table 26).

The half-life enhancing YTE mutation lowers the Tₘₗ value of FLT3L-Fc variants SEQ ID NOs: 9 and 14. Onset temperatures remain well above physiological temperature despite this shift (Table 26).

TABLE 26
Conformational Stability by Nano Differential Scanning Fluorimetry

| FLT3L-Fc Variant | | |
|---|---|---|
| SEQ ID NO: | Tₒₙ (□C) | Tₘ₁(□C) |
| 1 | 60.3 | 67.4 |
| 2 | 60.6 | 67.6 |
| 3 | 59.5 | 65.3 |
| 4 | 63.5 | 68.8 |
| 5 | 55.0 | 66.3 |
| 6 | 64.2 | 68.4 |
| 7 | 59.5 | 71.3 |
| 8 | 64.3 | 68.9 |
| 9 | 51.0 | 55.2 |
| 14 | 49.4 | 52.3 |

### Example 28

### FLT3L-Fc and Anti-PD1 Combination Study

In this example, we demonstrated the combined effects of anti-PD1 antibody (clone RMP1-14) and a FLT3L-Fc fusion protein using SEQ ID NO:20 (a murine surrogate) in a syngeneic mouse tumor model.

### Methods:

Eight-week old C57BL/6 mice (Jackson Laboratory) were inoculated with 2.5×10⁵ MC38 cells subcutaneously. Mice were then randomized when tumor volume reached 45-55 mm³ on day 0 and dosed intraperitoneally with FTL3L-Fc SEQ ID NO:20, anti-mouse PD-1 (clone RMP1-14) or an isotype control at the indicated concentrations and dosing frequencies. Tumors were measured at least 2 times a week using calipers. Tumor volumes were calculated using the following equation: (longest diameter*shortest diameter²)/2.

### Results

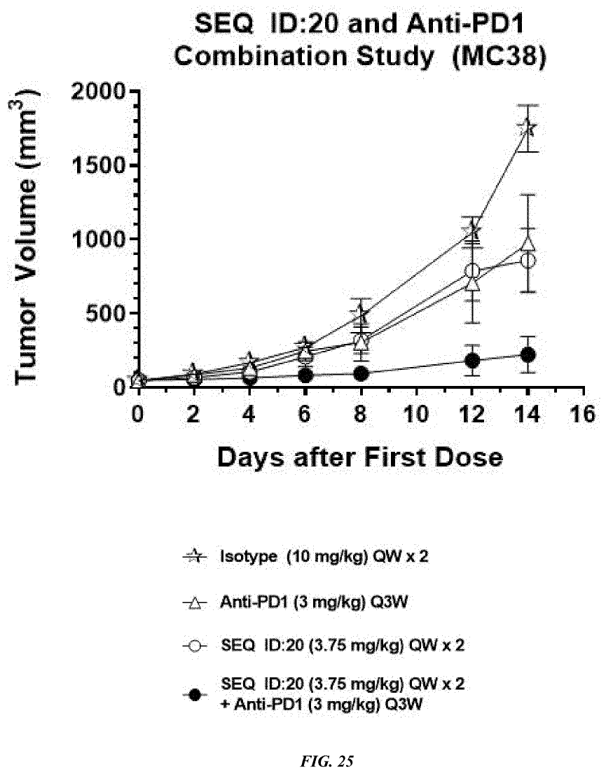

The results demonstrated a modest growth inhibition of MC38 tumors in C57BL/6 mice after single agent treatment with either one of the mouse FLT3-ligand Fc-fusion protein (SEQ ID NO:20) or anti-mouse PD-1, while combined treatment with the anti-PD1 antibody (clone RMP1-14) and the FLT3L-Fc fusion protein yielded strong tumor growth inhibition. The results are depicted in FIG. 25.

### Example 29

### FLT3L-Fc and Anti-CTLA4 Combination Study

In this example, we demonstrated the combined effects of anti-CTLA4 antibody (clone 9D9) and a FLT3L-Fc fusion protein using SEQ ID NO:20 (a murine surrogate) in a syngeneic mouse tumor model.

### Methods:

Nine-week old BALB/c mice (Taconic) were inoculated with 8×10⁵ CT26 cells subcutaneously. Mice were then randomized when tumor volume reached 60-70 mm³ on day 0 and dosed intraperitoneally with FTL3L-Fc SEQ ID NO:20, anti-mouse CTLA4 (clone 9D9) or an isotype control at the indicated concentrations and dosing frequencies. Tumors were measured at least 2 times a week using calipers. Tumor volumes were calculated using the following equation: (longest diameter*shortest diameter²)/2.

### Results:

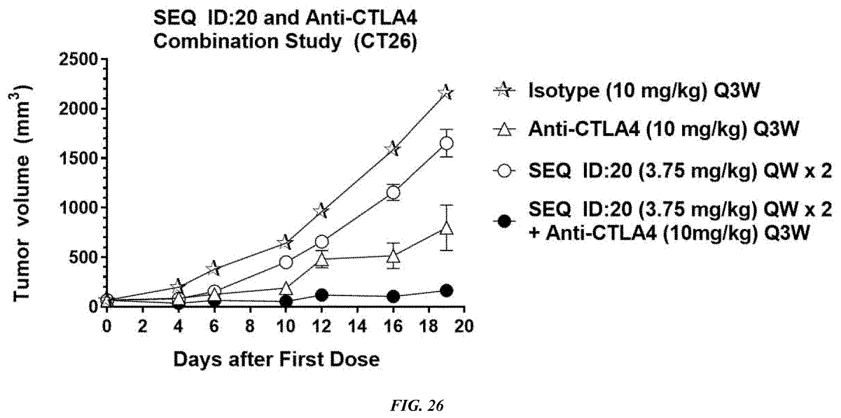

The results demonstrated a modest growth inhibition of CT26 tumors in BALB/c mice after single agent treatment with either one of the mouse FLT3-ligand Fc-fusion protein (SEQ ID NO:20) or anti-mouse CTLA4, while combined treatment with the anti-CTLA4 antibody (clone 9D9) and the FLT3L-Fc fusion protein yielded strong tumor growth inhibition. The results are depicted in FIG. 26.

### Example 30

### Effect of FLT3L on the Immunogenicity of an HBV Vaccine in a Mouse Model of Chronic HBV

We evaluated the potential effect of FLT3L on the immunogenicity of an HBV vaccine in the context of chronic HBV using an Adeno-Associated Virus (AAV)-HBV mouse model (Dion, et al., J Virol. (2013) 87(10):5554-63; and Yang, et al., Cell Mol Immunol. (2014) 11(1):71-8). Other immunomodulators including antibodies targeting PD-1, CTLA-4 and CD137 were also tested.

### Methods

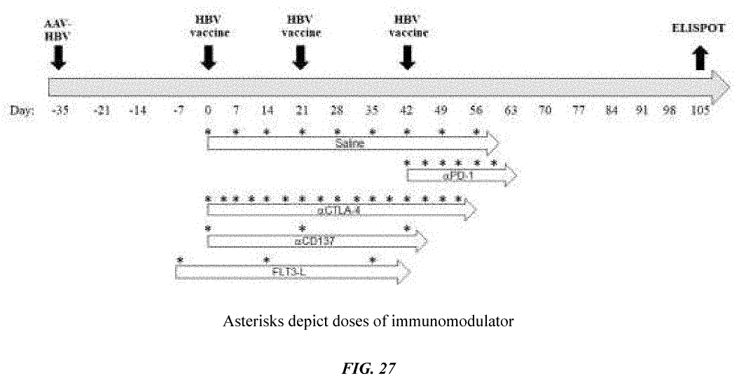

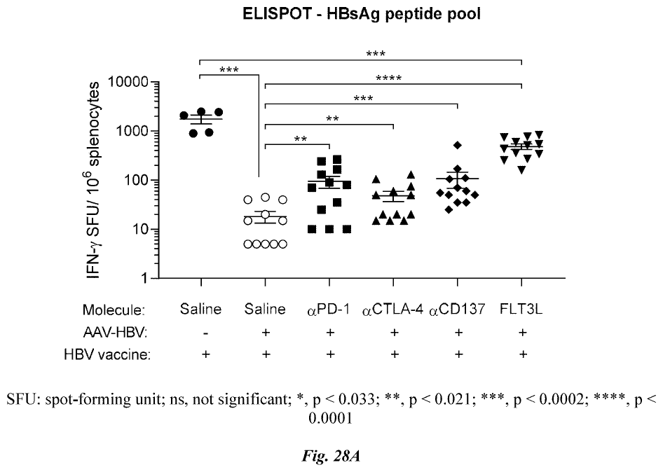

In this model, C57BL/6 mice were transduced with an AAV vector encoding a 1.2× length HBV genome (AAV-HBV mice), resulting in persistent HBV protein and virion production in hepatocytes, accompanied by antigenemia and viremia in serum. AAV-HBV mice were administered 3 doses of an HBV vaccine that is an arenavirus vector expressing HBV antigens including HBsAg, core and polymerase. Mice were treated with saline, mouse FLT3L, anti-mouse inhibitory PD-1, anti-mouse inhibitory CTLA-4 or anti-mouse stimulatory CD137 antibodies. A control group of mice received the HBV vaccine alone but no AAV-HBV to determine how the immunogenicity of the HBV vaccine was affected in the context of chronic HBV. HBV-specific IFN-γ ELISPOT was performed using spleens of all animals at the end of the study (day 105 post first vaccination). A diagram of this AAV-HBV immunogenicity study is shown in FIG. 27 and treatment groups are shown in Table 27. Data are expressed after subtraction of background signal in no-peptide control wells. Statistical analysis was performed using Mann-Whitney non-parametric test.

### Results

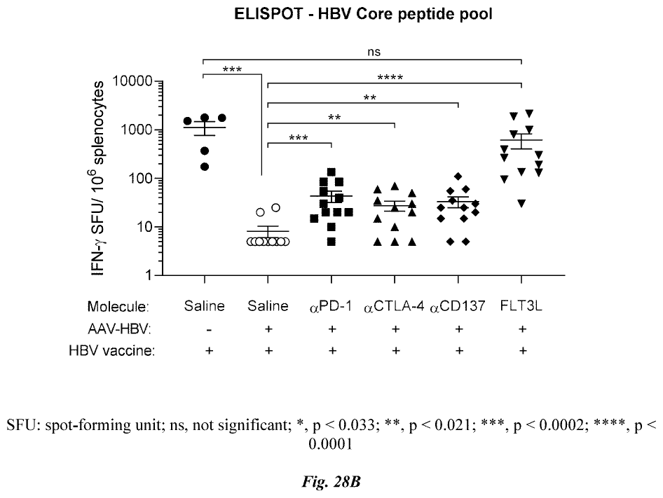

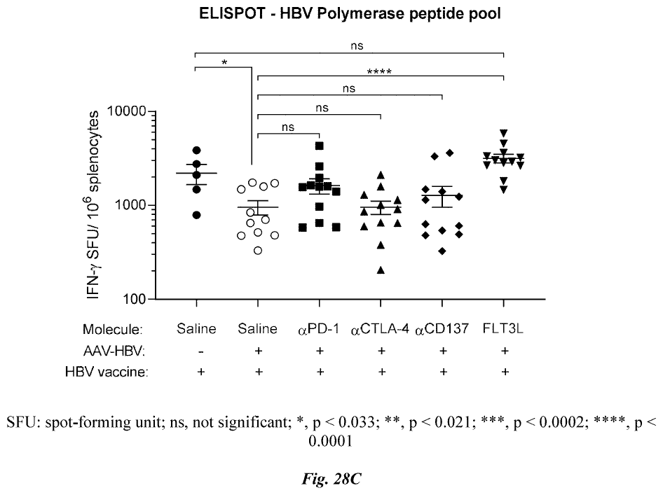

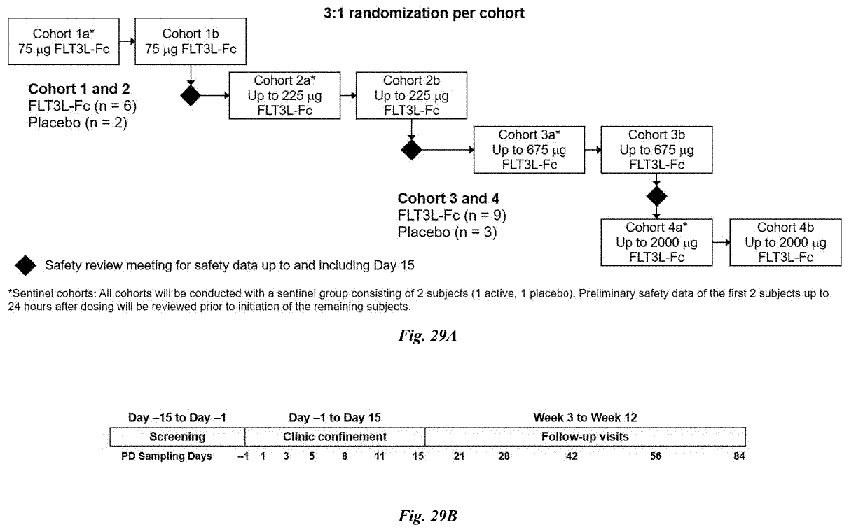

IFN-γ ELISPOT responses specific for HBsAg, HBV core and HBV polymerase are summarized in FIGS. 28A-C. Robust ELISPOT responses were observed for all 3 HBV antigens in mice without persistent HBV. In contrast, the ELISPOT responses obtained from AAV-HBV mice that received the HBV vaccine alone were significantly reduced, demonstrating T cell tolerance against the HBV proteins in AAV-HBV mice. In these mice, combined administration of FLT3L and HBV vaccine significantly increased the HBV-specific IFN-γ ELISPOT responses for all 3 HBV antigens. A comparable effect (except for HBV pol-specific responses) was observed with the other immunomodulators anti PD-1, anti CTLA-4 and anti CD137 antibodies although with lower magnitude.

TABLE 27
Study Groups in AAV-HBV Immunogenicity Study

| | | AAV- | HBV | | |
|---|---|---|---|---|---|
| Group | N | HBV | vaccine | Immunomodulator | Molecule and Dose |
| 1 | 11 | Yes | Yes | Vehicle | Saline |
| 2 | 12 | Yes | Yes | α-PD-1 | Clone RMP1-14 |
| | | | | | 8 mg/kg/dose |
| 3 | 12 | Yes | Yes | α-CTLA-4 | Clone 9D9 |
| | | | | | 10 mg/kg/dose |
| 4 | 12 | Yes | Yes | α-CD137 | Clone mAb8 |
| | | | | | 2.5 mg/kg/dose |
| 5 | 12 | Yes | Yes | FLT3L | Mouse surrogate of |
| | | | | | FLT3L-Fc |
| | | | | | (SEQ ID NO: 20) |
| | | | | | 1 mg/kg/dose |
| 6 | 5 | No | Yes | Vehicle | Saline |

### Example 31

### A Phase 1 Study in Healthy Volunteers to Evaluate the SingleDose Pharmacokinetics, Safety, and Tolerability of a FLT3L-Fc Fusion Protein

A study was conducted in healthy volunteeres (HVs) to evaluate the pharmacokinetics (PK), safety, and tolerability of escalating single doses of the FLT3L-Fc fusion protein comprising the amino acid sequence of SEQ ID NO: 14. An exploratory objective of this study is to evaluate the pharmacodynamics (PD) and PK/PD relationships for the FLT3L-Fc fusion protein.

### Methods

This was a first-in-human placebo-controlled study of a FLT3L-Fc fusion protein in healthy volunteers to evaluate the safety, PK, and PD of escalating single doses (ranging from 75 μg to 2000 μg) of the FLT3L-Fc fusion protein (SEQ ID NO: 14). The study was blinded to the subjects and the investigator. Each dose cohort enrolled 8-12 healthy subjects who received the FLT3L-Fc fusion protein or placebo as single IV infusion at 3:1 ratio. Subjects were observed in the Phase 1 unit for 15 days and then for 12 weeks as outpatients. As part of the PD evaluation, the changes in the number of cDC1s and cDC subtype 2 (cDC2) were investigated.

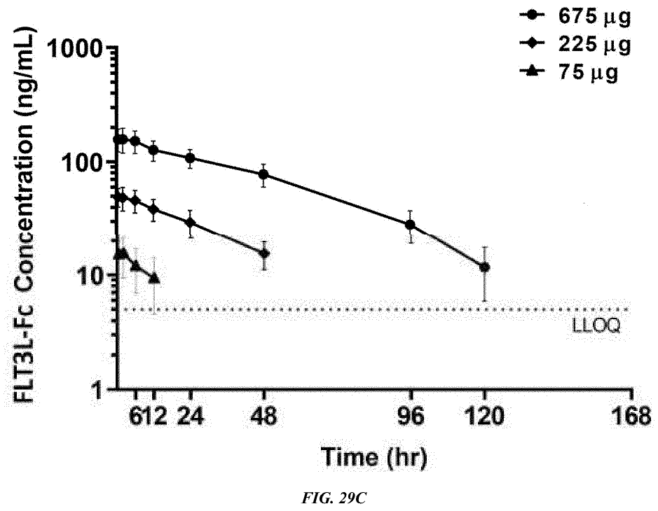

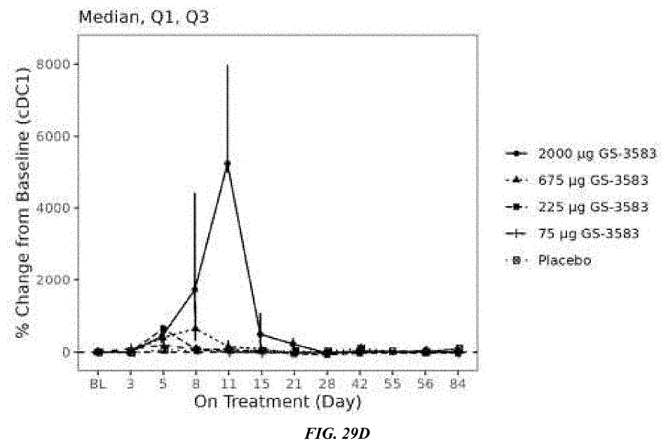

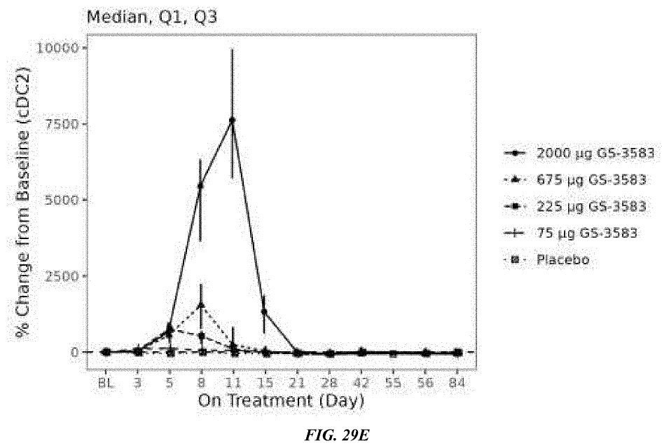

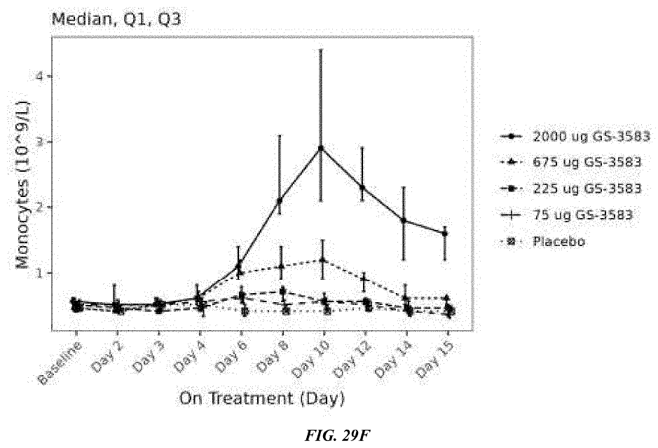

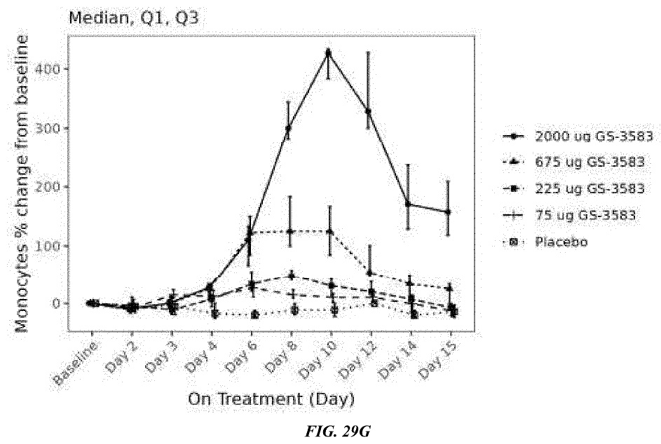

A high-level overview of the study design is described below and shown in FIG. 29A.

Healthy adults of ages 18-45 years were enrolled.

Screened subjects were admitted on Day −1 and confined to the study center until Day 15. Subjects returned for follow-up visits with discharge.

The FLT3L-Fc fusion protein or placebo was administered as an intravenous (IV) infusion on Day 1. The study treatments within each cohort are shown in Table 28 and the sampling days are shown in FIG. 29B.

TABLE 28
Study Treatments within Cohorts 1 to 4

| Cohort | Day 1 |
|---|---|
| 1 | 75 μg FLT3L-Fc fusion protein or placebo, administered IV as an infusion |
| 2 | Up to 225 μg^{a} FLT3L-Fc fusion protein or placebo, administered IV as an infusion |
| 3 | Up to 675 μg^{a} FLT3L-Fc fusion protein or placebo, administered IV as an infusion |
| 4 | Up to 2000 μg^{a} FLT3L-Fc fusion protein or |
| | placebo, administered IV as an infusion |

IV = intravenously;
PD = pharmacodynamics;
PK = pharmacokinetic
^{a}= the doses and number of cohorts may be adjusted based on the safety, tolerability, and available PK and PD data from previous dose groups in the study.

### Pharmacokinetic Assessments

### Serum Pharmacokinetic Collection

Serum concentrations of the FLT3L-Fc fusion protein was determined, and PK parameters were estimated. Intensive PK sampling occurred relative to the start time of infusion of the FLT3L-Fc fusion protein or placebo at Day 1 at predose (≤30 minutes before start of infusion), end of infusion, 2, 6, 12, 24, 48, 96, 120, 168, 240, and 336 hours. Additional samples will be collected at Days 21 (±1), 28 (±1), 42 (±1), 56 (±3), and 84 (±3) at the same time of day as start of infusion. An additional sample was collected at the ET visit (if applicable).

PD assessments

### PD Biomarkers

Whole blood biomarker samples were collected and PBMCs and plasma were isolated to measure PD biomarkers for the FLT3L-Fc fusion protein on Days −1, 1, 3, 5, 8, 11, 15, 21, 28, 42, 56, and 84 relative to the start of infusion. A separate blood sample to determine leukocyte count was drawn at the same time points. Additionally, serum was prepared from whole blood biomarker samples at the following time points:
Day −1: single sample
Day 1 at predose and 168 and 336 hours after the start of infusion
An additional sample was collected at the Day 28 (±1) visit

### Results

Safety, PK and PD data from cohorts 1 to 3 were determined. The subject characteristics and PD results for cohorts 1 to 4 are shown in Table 29.

TABLE 29

| | Cohort | | | |
|---|---|---|---|---|
| | 1 | 2 | 3 | 4 |
| Subjects treated | 8 | 8 | 12 | 12 |
| (A = active; P = placebo) | (6A; 2P) | (6A; 2P) | (9A; 3P) | (9A; 3P) |
| Age, years | 32 (20, 38) | 27 (23, 45) | 22 (18, 45) | 31.5 (18, 42) |
| median (range) | | | | |
| Male n (%) | 5 (62.5%) | 5 (62.5%) | 8 (66.7%) | 9 (75.0%) |
| cDC1 peak cell count* | Day 5 | Day 5 | Day 8 | Day 11 |
| median (Q1, Q3) | 69.6 (62.9, 89.2) | 169.0 (121.1, | 147.2 (130.1, | 922.2 (528.6, |
| | | 215.1) | 258.6) | 1355.2) |
| cDC1, fold change | Day 5 | Day 5 | Day 8 | Day 11 |
| from baseline* | 1.85 (1.38, 2.4) | 6.42 (5.62, 7.17) | 6.47 (3.29, 13.31) | 52.33 (50.01, 79.53) |
| median (Q1, Q3) | | | | |
| cDC2 peak cell count* | Day 5 | Day 5 | Day 8 | Day 11 |
| median (Q1, Q3) | 1346.0 (1124.8, | 2937.0 | 10677.6 | 36741.8 |
| | 1395.1) | (1679.8, | (7565.6, | (29835.8, |
| | | 3731.9) | 13702.6) | 55246.2) |
| cDC2, fold change | Day 5 | Day 5 | Day 8 | Day 11 |
| from baseline* | 1.20 (0.71, | 7.61 (3.21, | 17.30 (7.86, | 76.33 (57.33, |
| median (Q1, Q3) | 1.85) | 8.03) | 22.13) | 99.42) |

*Data shown only from the subjects who received FLT3L-Fc fusion protein (SEQ ID NO: 14); placebo data are excluded

Concentration-time profiles of FLT3L-Fc fusion protein following single IV infusion to healthy volunteers (cohorts 1 to 3) are shown in FIG. 29C. A summary of single-dose preliminary serum pharmacokinetic parameters of FLT3L-Fc fusion protein following administration of 75, 225, 675, or 2000 μg FLT3L-Fc fusion protein in healthy volunteers is shown in Table 30.

TABLE 30
Summary of Single-Dose Preliminary Serum Pharmacokinetic
Parameters of FLT3L-Fc Following Administration of 75, 225, or
675 μg FLT3L-Fc in Healthy Volunteers

| | Cohort 1: | Cohort 2: | Cohort 3: | Cohort 4: |
|---|---|---|---|---|
| | 75 μg | 225 μg | 675 μg | 2000 μg |
| PK Parameter | (N = 6) | (N = 6) | (N = 9) | (N = 9) |
| Cₘₐₓ (ng/mL) | 16 (35) | 51 (19) | 162 (24) | 570 (13) |
| Tₘₐₓ (h) | 2.0 (0.5, 2.0) | 0.5 (0.5, 2.0) | 2.0 (0.5, 6.0) | 0.5 (0.5, 6.0) |
| AUC_{inf} | NE* | NE* | 8840 (19) | 47900 (15) |
| (ng•h/mL) | | | | |
| t_{1/2} (h) | NE* | NE* | 34 (27, 42) | 53 (34, 74) |

Data presented as mean (% CV). Tₘₐₓ and t_{1/2} are presented as median (range).
*Parameters may not be robustly estimated because FLT3L-Fc concentrations fell below the LLOQ prior to the terminal phase for the 75 and 225 μg doses
NE = not estimated.

FIG. 29D shows a comparison of cDC1 cell quantitative changes in cohorts 1 to 4. FIG. 29E shows a comparison of cDC2 cell quantitative changes in cohorts 1 to 4. As shown in FIGS. 29D and E, a dose-dependent, transient increase in cDC1 cells and cDC2 cells was observed in healthy volunteers treated with FLT3L-Fc fusion protein.

FIGS. 29F-29G show changes in circulating monocytes over time in cohorts 1 to 4. FIG. 29F shows the number of monocytes over time in cohorts 1 to 4. FIG. 29G shows the percent change from baseline over time in cohorts 1 to 4. As shown in FIGS. 29F-29G, monocyte levels in FLT3L-Fc fusion protein treated healthy volunteeres increase over time with a peak at Day 10 and decline gradually from Day 10 to Day 15.

These results demonstrate that FLT3L-Fc fusion protein was well tolerated. For cohorts 1 to 4, there have been no serious or grade 3 or higher adverse events. Preliminary PK analysis suggested dose-dependent increase in FLT3L-Fc fusion protein exposure (AUC and Cₘₐₓ). Preliminary PD analysis shows that administration of FLT3L-Fc fusion protein resulted in dose-dependent increases in cDC1/cDC2 cells that peaked between days 5 to 11, with increasingly later peaks occurring at higher doses, and returned to baseline within three weeks of drug administration.

### Conclusions

FLT3L-Fc fusion protein single doses of up to 2,000 μg were well tolerated and there were no SAEs, deaths, or discontinuations due to AEs. FLT3L-Fc fusion protein induced dose dependent expansion of dendritic cells in the periphery. In patients with cancer, this increase in dendritic cells can be utilized to enhance anti-tumor immune responses to immuno-oncology therapies.

### Example 32

### A Phase 1b Dose Escalation Study to Evaluate the Safety, Tolerability, Pharmacokinetics, and Preliminary Efficacy of a FLT3L-Fc Fusion Protein in Subjects with Advanced Solid Tumors

A phase 1b, open-label, multicenter, dose-finding study was conducted to evaluate safety, tolerability, pharmacokinetics (PK), and preliminary efficacy of aFLT3L-Fc fusion protein comprising the amino acid sequence of SEQ ID NO: 14 as monotherapy in subjects with advanced solid tumors. The study also determined the maximum-tolerated dose (MTD) or recommended phase 2 dose (RP2D) of FLT3L-Fc fusion protein (SEQ ID NO: 14) as monotherapy in subjects with advanced solid tumors.

### Methods Summary

Approximately 33 adults aged 18 years with a histologically or cytologically confirmed locally advanced or metastatic malignant solid tumor that is refractory to or intolerant of standard therapy or for which no standard therapy is available will be enrolled. The study employs a 3+3 dose escalation design in which FLT3L-Fc fusion protein is administered intravenously for up to 52 weeks or until progressive disease (PD) or unacceptable toxicity. Up to five dose escalation cohorts have been planned. The MTD is the highest dose with incidence of dose-limiting toxicity (DLT) in <33% of 6 or more patients in the first 28 days of FLT3L-Fc fusion protein dosing; RP2D will be determined.

### Study Design

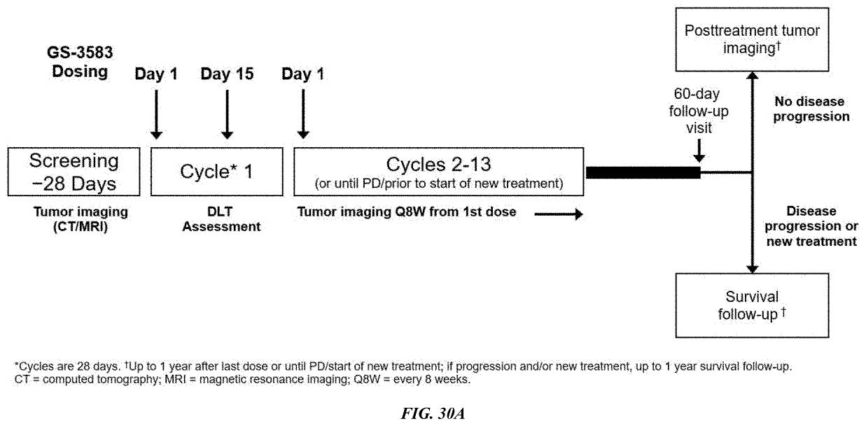

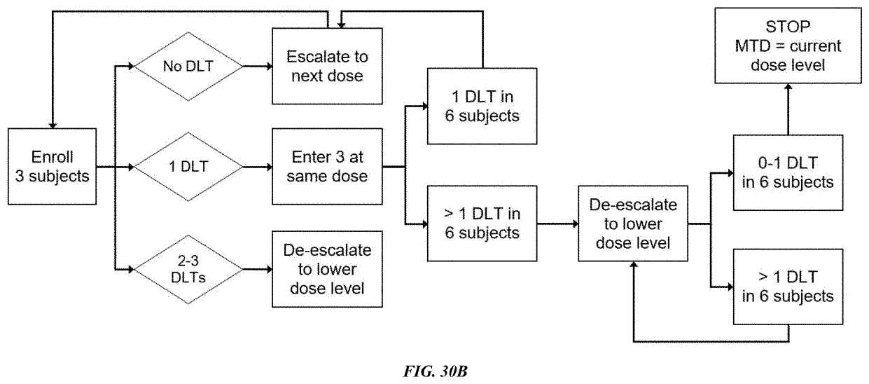

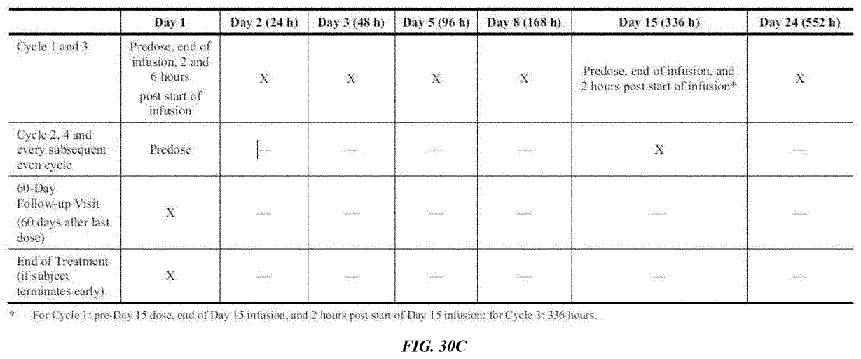

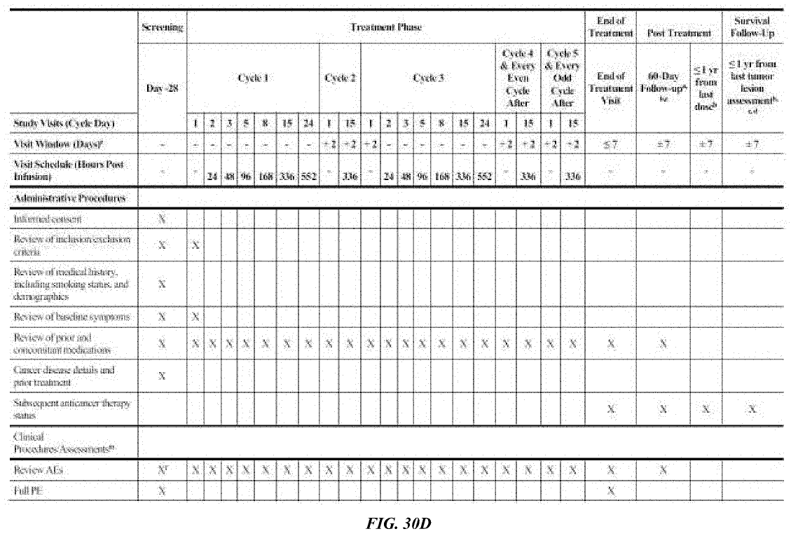

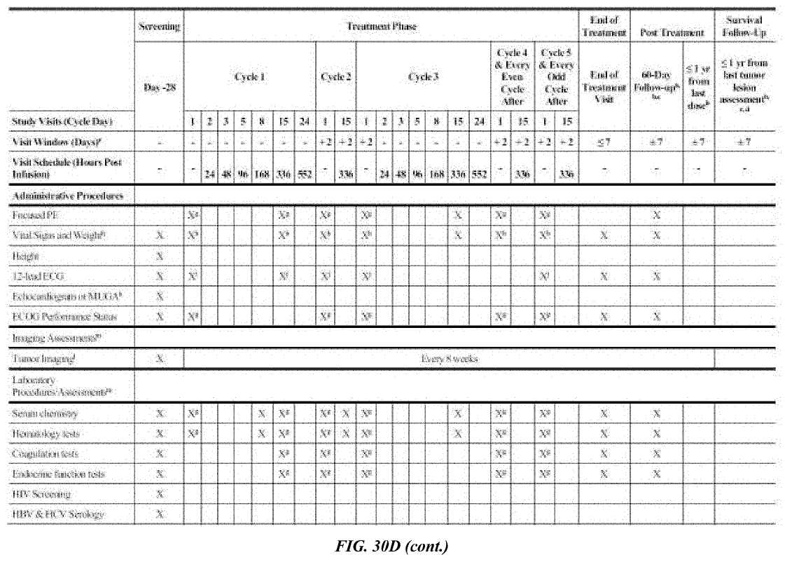

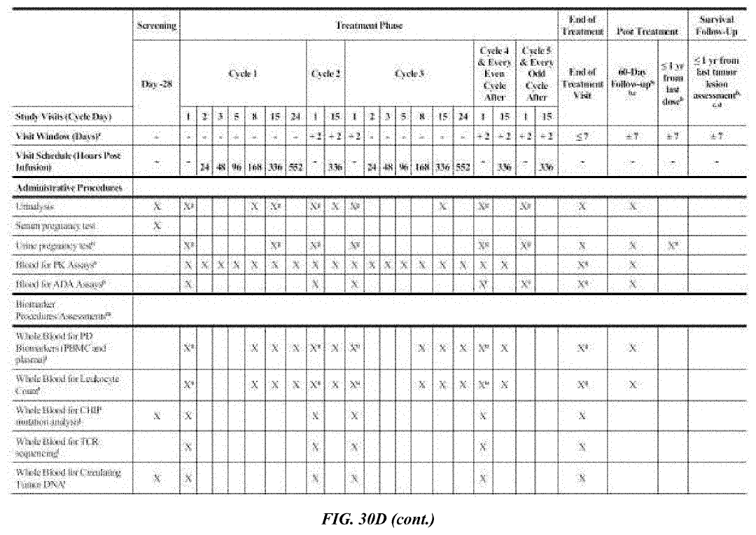

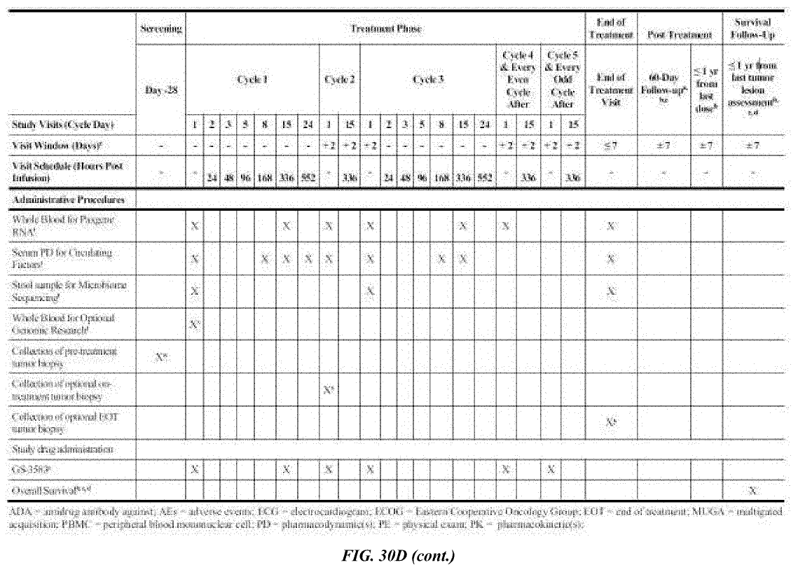

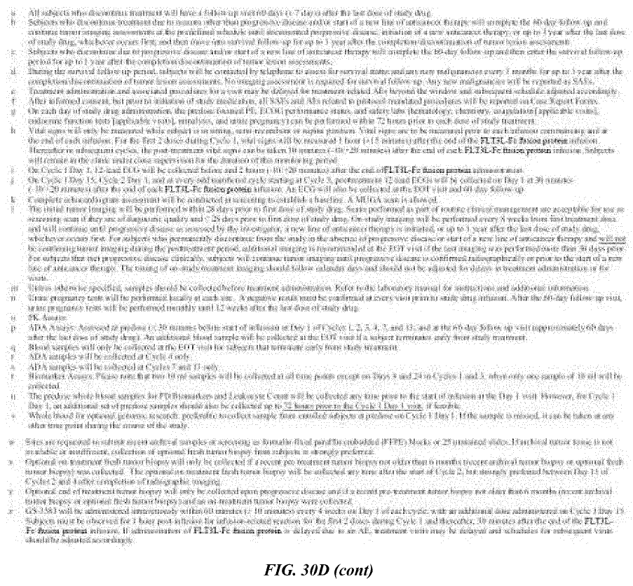

This is an open-label study to evaluate the safety, tolerability, PK, and preliminary efficacy of FLT3L-Fc fusion protein in subjects with advanced solid tumors to determine the MTD or RP2D level of FLT3L-Fc fusion protein as monotherapy. An overview of the study design is shown in FIG. 30A.

The study consists of a standard 3+3 dose escalation scheme with the following proposed escalating dose levels and schedules of FLT3L-Fc fusion protein: 675 μg, 2000 μg, 6,000 μg, 12,000 μg, and 20,000 μg administered on Days 1 and 15 of Cycle 1 and on Day 1 of each subsequent 4-week/28-day cycle.

### Objectives

The primary objectives of this study are as follows:
To characterize the safety and tolerability of FLT3L-Fc fusion protein as monotherapy in subjects with advanced solid tumors
To determine the MTD or RP2D of FLT3L-Fc fusion protein as monotherapy in subjects with advanced solid tumors

The secondary objectives of this study are as follows:
To characterize the PK of FLT3L-Fc fusion protein in subjects with advanced solid tumors
To evaluate the immunogenicity of FLT3L-Fc fusion protein in subjects with advanced solid tumors

The exploratory objectives of this study are as follows:
To evaluate the preliminary efficacy of FLT3L-Fc fusion protein as monotherapy in subjects with advanced solid tumors
To evaluate the effect of FLT3L-Fc fusion protein as monotherapy on PD markers in subjects with advanced solid tumors
To characterize dose- and/or exposure-response relationships of FLT3L-Fc fusion protein in subjects with advanced solid tumors
To characterize immunologic changes associated with FLT3L-Fc fusion protein as monotherapy and correlation with clinical response in subjects with advanced solid tumors

### Endpoints

Primary, secondary, and other endpoints of this study are described below.

Primary endpoints:
a. Incidence of DLTs
b. Incidence of adverse events and laboratory abnormalities by National Cancer Institute Common Terminology Criteria for Adverse Events (NCI CTCAE) v5.0

Secondary endpoints:
a. FLT3L-Fc PK parameters

Other endpoints
a. Overall response rate (ORR) and confirmed ORR, per Response Evaluation Criteria in Solid Tumors (RECIST) 1.1
b. Progression-free survival
c. Duration of response
d. Time to response
e. Overall survival
f Changes in pharmacodynamic (PD) markers of FLT3L-Fc fusion protein
g. Other PK parameters of interest
h. Correlations between FLT3L-Fc fusion protein PK and PD

Assessments include safety, PK, PD, including cDCs, immunogenicity, and efficacy by RECIST 1.1 in CT/MRI imaging.

### Dose Escalation

Subjects with advanced solid tumors who have failed or are intolerant to standard therapy or for whom no standard therapy exists will be sequentially enrolled at progressively higher dose levels to receive FLT3L-Fc fusion protein as monotherapy.

Dose escalation will proceed using a standard 3+3 design as shown in FIG. 30B. The starting dose will be 675 μg and may be modified if the results of the Phase 1a healthy volunteer study (Study GS-US-496-5619) are available prior to the initiation of this study. Subsequent doses of 2000 μg, 6000 μg, 12,000 μg, and 20,000 μg are planned. Dose level increases will be 3-fold or less (Table 31). FLT3L-Fc fusion protein will be administered on Days 1 and 15 of Cycle 1 and on Day 1 of each subsequent 4-week/28-day cycle for up to 52 weeks or until the subject meets study treatment discontinuation criteria.

TABLE 31
Dose Escalation Dose Levels
Dose Level Schema

| Cohort | FLT3L-Fc fusion protein (μg) | Fold Increase |
|---|---|---|
| 1 | 675 | — |
| 2 | 2000 | ≤3× |
| 3 | 6000 | ≤3× |
| 4 | 12000 | ≤2× |
| 5 | 20000 | ≤1.67× |

DLTs will be assessed during the first 28 days of therapy at each dose level. Dose escalation will occur if no subjects experience a DLT. If 1 subject experiences a DLT, an additional 3 subjects will be enrolled. If 2 subjects experience a DLT, dose de-escalation will occur. A minimum of 6 subjects need to be treated at a dose level before the dose can be deemed as the MTD.

### Dose Escalation Criteria

For any given cohort, the sponsor may elect to hold dosing, select an intermediate dose, or stop study enrollment at any time based on review of the preliminary safety data.

Based on review of relevant safety and PK data by the SRT (safety review team) and in discussion with the investigator, escalation to a higher dose will occur only in the absence of DLTs and/or meeting any prespecified stopping criteria.

Dose Limiting Toxicity (DLT) Definition

A DLT is any toxicity defined below occurring with FLT3L-Fc fusion protein monotherapy during the DLT assessment period (from Day 1 through Day 28) considered at least possibly related to FLT3L-Fc fusion protein monotherapy.

A DLT may lead to permanent withdrawal of FLT3L-Fc fusion protein for the subject after discussion between the investigator and sponsor. Study drug related AEs requiring permanent treatment discontinuation are listed in Table 33.

1) Hematologic
Grade≥3 thrombocytopenia with clinically significant bleeding (ie, requires hospitalization, transfusion of blood products, or other urgent medical intervention)
Grade≥3 febrile neutropenia (absolute neutrophil count [ANC]<1.0×109/L and fever>101° F./38.3° C.)
Any Grade 4 hematologic laboratory abnormalities/adverse events (AEs) regardless of duration will be considered DLTs with the exception of:
   Grade 4 lymphopenia
   Grade 4 neutropenia lasting ≤7 days that is not associated with fever (the use of growth factors is permitted)
   Grade 4 anemia explained by underlying disease

2) Non-Hematologic
Grade≥3 non-hematologic toxicity, except:
   Transient (≤3 days) Grade 3 fatigue, local reactions, headache, nausea, emesis, or diarrhea that are controlled with medical management and/or resolves to Grade≤1 or baseline
   Any Grade 3 endocrinopathy that is adequately controlled by hormonal replacement
   Grade 3 AE of tumor flare (defined as local pain, irritation, or rash localized at sites of known or suspected tumor)
   Transient (≤3 days) Grade 3 flu-like symptoms or fever, which are controlled with medical management
   An event clearly associated with the underlying disease, progressive disease, a
   concomitant medication, or comorbidity
Grade≥2 non-hematologic treatment-emergent adverse event (TEAE) that in the opinion of the investigator is of potential clinical significance such that further dose escalation would expose subjects to unacceptable risk
Any Grade 3 or Grade 4 elevation in aspartate aminotransferase (AST) or alanine
aminotransferase (ALT) associated with a Grade 2 elevation in bilirubin lasting ≥7 days
Any irAE for which FLT3L-Fc fusion protein should be permanently discontinued as described in Table 33 (eg, any grade encephalitis, Grade≥3 myocarditis, reoccurrence of the same Grade 3 adverse reaction)

3) Dosing/Procedures-Related Toxicities
Inability to receive the first 2 doses of FLT3L-Fc fusion protein in Cycle 1 because of related toxicity, even if the toxicity does not meet DLT criteria defined above (regardless of dosing schedule)
Note: Exceptions include DLT exclusions mentioned above.

4) Grade 5 Event (ie, Death)

### Duration of Treatment

FLT3L-Fc fusion protein will be administered up to a total duration of 52 weeks. Subjects will be treated with study drug until unacceptable toxicity, progressive disease, or other reasons for discontinutation of study treatment listed below:
Intercurrent illness that would, in the judgment of the investigator, affect assessments of clinical status to a significant degree.
Unacceptable toxicity, or toxicity that, in the judgment of the investigator, compromises the ability to continue study-specific procedures or is considered not to be in the subject's best interest
Progressive Disease
Initiation of a new anti-cancer Therapy
Death
Lost to follow-up
Subject request to discontinue for any reason
Investigator's discretion
Protocol Violation or major protocol deviation
Pregnancy during the study
Discontinuation of the study at the request of sponsor, a regulatory agency, or an IRB or IEC

### Dosing and Administration of FLT3L-Fc Fusion Protein

FLT3L-Fc fusion protein will be administered as an IV infusion over 60 (±10) minutes. The proposed dose and the dosing frequency of FLT3L-Fc fusion protein are 675 μg, 2000 μg, 6000 μg, 12,000 μg, and 20,000 μg administered on Days 1 and 15 of Cycle 1 and on Day 1 of each subsequent 4-week/28-day cycle.

Premedications should not be administered routinely prior to dosing of FLT3L-Fc fusion protein unless a previous infusion reaction occurred. Refer to Treatment of Infusion-Related Reactions section and Table 32 for subsequent premedication recommendations following FLT3L-Fc fusion protein-related infusion reactions.

FLT3L-Fc fusion protein should be administered IV over approximately 60 minutes (±10 minutes) at the research clinic by a qualified staff member. Infusions will be followed immediately with a saline flush of the IV line, per institutional guidelines. Modifications of the infusion rate due to infusion-related reactions are described in the Treatment of Infusion-Related Reactions section and Table 32. The study drug will be administered without regard to food.

Subjects receiving FLT3L-Fc fusion protein should be monitored for infusion-related reactions. This includes the measurement of vital signs prior to each infusion commencing and at the end of each infusion. For the first 2 doses during Cycle 1, vital signs will be measured 1 hour (±15 minutes) after the end of the FLT3L-Fc fusion protein infusion. Thereafter in subsequent cycles, the post-treatment vital signs can be taken 30 minutes (−10/+20 minutes) after the end of the FLT3L-Fc fusion protein infusion. Subjects will remain in the clinic under close supervision for the duration of this monitoring period.

### Dose Modifications and Treatment Delay

### Dose Modifications

Intrasubject dose reduction of FLT3L-Fc fusion protein is not permitted; the need for a dose reduction is considered a DLT, and the subject will be discontinued from treatment. Intrasubject dose escalation of FLT3L-Fc fusion protein may be permitted once the MTD has been determined, at investigator's discretion.

### Treatment Delay

Study treatment may be delayed due to any AE, laboratory abnormality, or intercurrent illness which, in the judgment of the investigator, warrants a delay.

Subjects who have drug-related toxicities that meet the criteria for dose delay should have study drug treatment delayed until criteria to resume treatment are met. Subjects who fail to receive 3 or more consecutive doses of FLT3L-Fc fusion protein due to treatment delay will be discontinued from the study unless agreed otherwise with the sponsor medical monitor.

### Treatment of Infusion-Related Reactions

Subjects receiving FLT3L-Fc fusion protein should be monitored for infusion-related reactions. This includes the measurement of vital signs prior to each infusion commencing and at the end of each infusion. For the first 2 doses during Cycle 1, vital signs will be measured 1 hour (±15 minutes) after the end of the FLT3L-Fc fusion protein infusion. Thereafter in subsequent cycles, the post-treatment vital signs can be taken 30 minutes (−10\+20 minutes) after the end of the FLT3L-Fc fusion protein infusion. Subjects will remain in the clinic under close supervision for the duration of this monitoring period. Subjects with mild or moderate infusion-related reactions may receive FLT3L-Fc fusion protein with close monitoring.

Premedication with an antipyretic or antihistamine for subsequent treatment administration may be considered. For severe infusion-related reactions, FLT3L-Fc fusion protein infusion must be discontinued, and appropriate medical therapy should be administered (Table 32).

TABLE 32
FLT3L-Fc Infustion-Related Reactions Management Guidelines

| | | Premedication |
|---|---|---|
| | | at Subsequent |
| | | Dose |
| CTCAE Grade | Treatment | Administration |
| Grade 1 | Increase monitoring of vital signs as medically | None |
| Mild transient reaction; | indicated until the subject is deemed | |
| infusion | medically stable | |
| interruption not | in the opinion of the investigator. | |
| indicated; intervention | | |
| not indicated. | | |
| Grade 2 | Stop infusion and monitor symptoms. | Subject may be |
| Requires infusion | Additional appropriate medical therapy may | premedicated |
| interruption but | include | prior to infusion |
| responds promptly to | but is not limited to the following: | with the |
| symptomatic treatment | IV fluids | following: |
| (eg, antihistamines, | Antihistamines | Diphenhydramine |
| nonsteroidal anti- | NSAIDs | 25-50 mg PO (or |
| inflammatory drugs | Acetaminophen | equivalent dose |
| [NSAIDs], narcotics, IV | Narcotics | of |
| fluids); | Increase monitoring of vital signs as medically | antihistamine). |
| prophylactic medications | indicated until the subject is deemed | Acetaminophen |
| indicated for ≤ 24 hours. | medically stable | 500-1000 mg PO |
| | in the opinion of the investigator. | (or equivalent |
| | If symptoms resolve within 1 hour of stopping | dose of |
| | drug infusion, the infusion may be | antipyretic). |
| | restarted at 50% of the original infusion | |
| | rate. Otherwise, dose administration will | |
| | be held until symptoms resolve, and the | |
| | subject should be premedicated for the | |
| | next scheduled dose. Subjects who develop | |
| | Grade 2 toxicity despite adequate | |
| | premedication should be permanently | |
| | discontinued from further study drug | |
| | administration. | |
| Grade 3 or 4 | Stop infusion. | No subsequent |
| Grade 3 | Additional appropriate medical therapy may | dosing. |
| Prolonged (ie, not | include | |
| rapidly responsive to | but is not limited to: | |
| symptomatic | IV fluids | |
| medication and/or brief | Antihistamines | |
| interruption of infusion); | NSAIDs | |
| recurrence of symptoms | Acetaminophen | |
| following initial | Narcotics | |
| improvement; | Oxygen | |
| hospitalization indicated | Pressor | |
| for other | Corticosteroids | |
| clinical sequelae (eg, | Epinephrine | |
| renal impairment, | Increase monitoring of vital signs as medically | |
| pulmonary | indicated until the subject is deemed | |
| infiltrates). | medically stable in the opinion of the | |
| Grade 4 | investigator. | |
| Life-threatening; pressor | Hospitalization may be indicated. | |
| or | Subject is permanently discontinued from | |
| ventilatory support | further study drug administration. | |
| indicated. | | |

CTCAE = Common Terminology Criteria for Adverse Events;
IV = intravenous;
NSAID = nonsteroidal anti-inflammatory drug;
PO = orally

Subjects who do not experience any infusion-related reactions of Grade 1 or higher during or after the infusion may be released from monitoring after 1 hour if they are otherwise stable. Subjects with any infusion-related reactions must be managed as per the guidelines in Table 32, and monitoring will continue until any infusion-related reactions have abated to less than Grade 1 and at least 1 hour has passed from the completion of the entire infusion and flushing the line.

Acute infusion-related reactions (which can include cytokine release syndrome, angioedema, or anaphylaxis) are different from allergic/hypersensitive reactions, although some of the manifestations are common to both AEs. Signs and symptoms usually develop during or shortly after drug infusion and generally resolve completely within 24 hours of completion of infusion. Signs/symptoms may include allergic reaction/hypersensitivity (including drug fever); arthralgia (joint pain); bronchospasm; cough; dizziness; dyspnea (shortness of breath); fatigue (asthenia, lethargy, malaise); headache; hypertension; hypotension; myalgia (muscle pain); nausea; pruritus/itching; rash/desquamation; rigors/chills; sweating (diaphoresis); tachycardia; tumor pain (onset or exacerbation of tumor pain due to treatment); urticaria (hives, welts, wheals); and vomiting.

Table 32 shows treatment guidelines for subjects who experience an infusion-related reaction associated with administration of FLT3L-Fc fusion protein. For individual subjects, once the FLT3L-Fc fusion protein infusion rate has been decreased by 50% or interrupted due to an infusion-related reaction, it must remain decreased for all subsequent infusions for that subject. If the subject has a second infusion related reaction that is Grade≥2 at the slower infusion rate, then infusion should be stopped, and the subject should permanently discontinue treatment. If a subject experiences a Grade 3 or Grade 4 infusion-related reaction at any time, the subject must permanently discontinue treatment (Table 32). If an infusion-related reaction occurs, all details about drug preparation and infusion must be recorded.

Clinically significant, abnormal 12-lead safety electrocardiograms (ECGs) should be repeated. Subjects who have 2 consecutive ECGs showing a new absolute QTcF duration>500 ms, or a QTc>60 ms over the corresponding baseline value must discontinue any medications that could prolong the QT interval. Subject's concomitant medications should be reviewed to determine a potential etiology for the ECG changes. Appropriate intervention (ie, cardiology evaluation, telemetry monitoring, management of electrolyte abnormalities) in response to treatmentemergent QT interval prolongation should be initiated.

Should infusion-related reactions be considered a significant safety issue by the SRT, the SRT may decide to recommend premedication with an antihistamine and acetaminophen approximately 30 to 60 minutes before each dose of FLT3L-Fc fusion protein (eg, 25-50 mg diphenhydramine, 500-1000 mg acetaminophen or equivalent dose of antipyretic). This regimen may be modified based on local treatment standards and guidelines, as appropriate.

All subjects will be given information on and instructions regarding both infusion-related reactions (which are expected to be most likely in the hour after completion of the infusion) and adverse events with a potential immunologic etiology (irAEs) before leaving the study site.

### Criteria to Resume Treatment

Subjects may resume treatment with FLT3L-Fc fusion protein when drug-related AE(s) resolve(s) to Grade 1 or baseline value, with the following clarifications and certain exceptions:
Subjects may resume treatment in the presence of Grade 2 fatigue.
Subjects with combined drug-related elevations in AST or ALT>3×ULN and total bilirubin>2×ULN for longer than 7 days should have their treatment
permanently discontinued.
Drug-related Grade 2 pulmonary toxicity or colitis must have resolved to baseline before treatment is resumed.
Drug-related endocrinopathies adequately controlled with only physiologic hormone
replacement may resume treatment. (Exception: Study treatment does not need to be delayed/interrupted for subjects who experience immune-related hypothyroidism [Grades 1-2].)
Subjects who received systemic corticosteroids for management of any drug-related toxicity must be off corticosteroids or have tapered down to an equivalent dose of prednisone≤10 mg/day.

### Study Discontinuation Criteria

A subject will be discontinued from the study for any of the following reasons:
Withdrawal of consent
Investigator's discretion
Death
Lost to follow-up
Discontinuation of the study at the request of sponsor, a regulatory agency, or an institutional review board (IRB) or independent ethics committee (IEC)

### Permanent Treatment Discontinuation Due to Adverse Events

Study drug treatment must be discontinued for drug-related AEs described in Table 33.

TABLE 33
Drug-Related Adverse Events Requiring Discontinuation of FLT3L-Fc Fusion Protein

| Toxicity Category* | Quantifying Severity/Duration |
|---|---|
| Ophthalmic | Grade 2 uveitis, blurred vision, eye pain, and/or reduction of |
| | visual acuity that does not respond to topical therapy and does not |
| | improve to Grade 1 severity within 2 weeks OR requires systemic |
| | treatment |
| | Grade 3 or higher uveitis |
| Gastrointestinal | Grade 3 or higher diarrhea, colitis |
| Neurologic | Grade 3 or higher neurologic toxicities |
| Dermatologic | Grade 3 or higher skin AE, suspected Stevens-Johnson syndrome, |
| | or toxic epidermal necrolysis |
| Pulmonary | Grade 2 pneumonitis or interstitial lung disease that does not |
| | improve after 2 weeks or worsening despite dose delay and |
| | systemic corticosteroids |
| | Grade 3 or higher pneumonitis |
| Endocrinopathies | Grade 3 endocrinopathies not adequately controlled with |
| | physiologic hormone replacement |
| | Grade 4 or higher endocrinopathies |
| Hypersensitivity | Grade 3 or higher bronchospasm, hypersensitivity reaction, or |
| | infusionrelated reaction |
| Laboratory | Thrombocytopenia |
| | Grade 3 thrombocytopenia lasting > 7 days or associated with |
| | clinically significant bleeding (ie, requires hospitalization, |
| | transfusion of blood products, or other urgent medical |
| | intervention) |
| | Grade 4 thrombocytopenia |
| | Neutropenia |
| | Grade 4 neutropenia lasting > 7 days |
| | Electrolyte Abnormalities |
| | Grade 4 electrolyte abnormalities with clinical sequelae |
| | Grade 4 electrolyte abnormalities unable to be corrected with |
| | supplementation/appropriate management within 72 hours of |
| | onset |
| | Liver Function Abnormalities |
| | AST or ALT > 5 × ULN |
| | Total bilirubin > 3 × ULN |
| | Concurrent AST or ALT > 3 × ULN AND total bilirubin > 2 × |
| | ULN lasting > 7 days |
| Other | Grade 2 or 3 adverse event (laboratory or non-laboratory) that |
| | does not resolve in ≤ 12 weeks |
| | Recurrence of Grade 3 or higher adverse event (laboratory or |
| | nonlaboratory) |
| | Grade 4 non-hematologic adverse event |

### Treatment Beyond Initial Progressive Disease

Subjects who experience initial radiologic progressive disease and are clinically worsened will discontinue study drug treatment and no further imaging is required.

Subjects who experience initial radiologic progressive disease but improve clinically are considered to have initial RECIST 1.1-defined progressive disease and will be permitted, with sponsor's approval, to continue with study drug treatment. These subjects will be reevaluated using the same imaging modality no less than 4 weeks later (after the last imaging with initial RECIST 1.1-defined progressive disease) to assess whether study drug treatment will be continued. To continue study drug treatment beyond initial RECIST 1.1-defined progressive disease, they must meet all the following criteria:
There is investigator-assessed clinical benefit from the study drug treatment
Subject is clinically stable
Subject is tolerating study drug, and
There is an agreement with sponsor

The assessment of clinical benefit should take into account whether the subject is clinically deteriorating and unlikely to receive further benefit from continued study drug treatment. The following criteria need to be taken into consideration:
Absence of clinical symptoms and signs (including worsening of laboratory values) indicating progressive disease,
No decline in ECOG performance status attributed to underlying malignancy, and
Absence of rapid progression of disease or of progressive tumor at critical anatomical sites (eg, spinal cord compression) requiring urgent alternative medical intervention.

### End of Study

End of study will be defined as when the last subject reaches the last scheduled follow-up time point (including the 60-day follow-up or survival follow-up, whichever occurs the latest), or is lost to follow-up, withdraws from the study, death, or the time at which the sponsor closes the study.

### Poststudy Care

Upon discontinuation from study treatment, subjects will receive the care upon which they and their physician(s) agree. Subjects will be followed for survival and AEs as specified in the Adverse Events and Serious Adverse Events described below.

### Adverse Events

Following initiation of study medication, collect all AEs, regardless of cause or relationship, until 60 days after the last administration of study drug and report them on the eCRFs as instructed.

All AEs should be followed until resolution or until the AE is stable, if possible. Sponsor may request that certain AEs be followed beyond the protocol-defined follow-up period

### Serious Adverse Events

All SAEs, regardless of cause or relationship, that occur after the subject first consents to participate in the study (i.e., signing the ICF) and throughout the duration of the study, including the 60-day follow-up visit, must be reported on the applicable electronic case report forms (eCRFs) and sponsor as instructed below in this section. This also includes any SAEs resulting from protocol-associated procedures performed after the informed consent form is signed.

Any SAEs and deaths that occur within 60 days of the last dose of study drug, regardless of causality, should also be reported.

Investigators are not obligated to actively seek SAEs after the 60-day follow-up visit; however, if the investigator learns of any SAEs that occur after the protocol-defined follow-up period has concluded and the event is deemed relevant to the use of study drug, the investigator should promptly document and report the event to sponsor.

All new malignancies, other than which was being studied during treatment with FLT3L-Fc fusion protein, that occur during the study or the posttreatment/survival follow-up period up to 1 year after the completion/discontinuation of lesion tumor assessments will be deemed medically important and reported as an SAE.

### Subject Population

This study will enroll approximately 33 subjects with advanced solid tumors.

A subject who fails to receive all FLT3L-Fc fusion protein treatments or fails to complete all safety assessments in the DLT period for reasons other than DLT will be replaced.

### Key Inclusion Criteria

Subjects must meet all of the following inclusion criteria to be eligible for participation in this study:
a. Histologically or cytologically confirmed locally advanced or metastatic malignant solid tumor that is refractory to or intolerant of standard therapy or for which no standard therapy is available
b. Measurable disease on imaging based on RECIST 1.1
c. Eastern Cooperative Oncology Group performance status of 2
d. Life expectancy of 3 months in the opinion of the investigator
e. Adequate organ function as assessed by hematological, renal, and hepatic parameters, and no clinically significant coagulopathy as indicated by the laboratory values in Table 34.

TABLE 34

| System | Laboratory Values |
|---|---|
| Hematological | |
| Absolute Neutrophil Count (ANC) | ≥ 1.5 × 10⁹/L |
| Platelets | ≥ 100 × 10⁹/L |
| Hemoglobin | ≥ 8 g/dL (≥ 10 g/dL in patients with |
| | cardiac disease) |
| Renal | |
| Creatinine Clearance | ≥ 50 mL/min by the Cockcroft- |
| | Gault method |
| Hepatic | |
| Total bilirubin | ≤ 1.5 × ULN |
| AST (SGOT) and ALT (SGPT) | ≤ 3 × ULN (≤ 5 × ULN in patients |
| | with liver metastases) |
| Coagulation | |
| International Normalized Ratio | ≤1.5 ULN unless the subject is |
| (INR) | receiving anticoagulant therapy |
| or Prothrombin Time (PT) | |
| Activated Partial Thromboplastin | ≤1.5 □ ULN unless the subject is |
| Time (aPTT) | receiving anticoagulant therapy |

ALT = alanine aminotransferase;
AST = aspartate aminotransferase;
SGOT = serum glutamic oxaloacetic transaminase;
SGPT = serum glutamic pyruvic transaminase;
ULN = upper limit of normal
a. All screening laboratory tests must be reviewed by the investigator and be acceptable prior to enrollment.
b. Hematologic laboratory values must be met at screening visit and maintained without transfusion and growth factors prior to the first dose of study drug.
c. Subjects on full-dose oral anti coagulation, must be on a stable dose (minimum duration 14 days prior to the Screening Visit). Subjects on low molecular weight heparin will be allowed. In subjects receiving warfarin, the recommended INR is ≤ 3.0 with no active bleeding (ie, no bleeding within 14 days prior to first dose of study drug).

### Key Exclusion Criteria

Subjects who meet any of the following exclusion criteria are not eligible to be enrolled in this study:
a. Prior systemic cytotoxic chemotherapy, biological therapy, radiotherapy, or major surgery within 3 weeks of Cycle 1 Day 1; a 1-week washout is permitted for palliative radiation to non-central nervous system (CNS) disease with sponsor approval
b. Known severe hypersensitivity reactions (NCI CTCAE Grade 3) to fully human monoclonal antibodies or fusion proteins, FLT3L-Fc fusion protein formulation excipients, or severe reaction to immuno-oncology agents, such as colitis or pneumonitis requiring treatment with corticosteroids, any history of anaphylaxis, or uncontrolled asthma
c. Concurrent active malignancy other than nonmelanoma skin cancer, carcinoma in situ of the cervix, or superficial bladder cancer who has undergone potentially curative therapy with no evidence of disease. Other previous malignancies are allowed if disease free for >2 years
d. History of hematological malignancy, monoclonal gammopathy of undetermined significance, or other preleukemic states
e. Known CNS metastasis(es), unless metastases are treated and stable and do not require systemic corticosteroids at least 1 week prior to study treatment. Individuals with a history of carcinomatous meningitis are excluded regardless of clinical stability
f. Active or history of autoimmune disease that has required systemic treatment within 2 years of the start of study treatment

### Rationale for the Study

The therapeutic hypothesis of FLT3L-Fc fusion protein is that FLT3 agonism will convert cold, uninflamed tumors into warm and hot, T-cell inflamed tumors. Cold tumors are not sensitive to PD-L1 blockade; however, FLT3L-Fc fusion protein treatment is expected to enhance the efficacy of PD-L1 blockade due to the resulting T-cell infiltration into the tumor in response to DC recruitment and expansion of intratumoral DCs. The current study GS-US-496-5657 is designed to assess the safety, tolerability, PK, and preliminary efficacy of FLT3L-Fc fusion protein given as a monotherapy and determine the maximum tolerated dose (MTD). The study will also explore the PK and PK-PD relationship as evaluated by peripheral DC expansion. Once the MTD of FLT3L-Fc fusion protein has been determined, future studies will explore the combination of FLT3L-Fc fusion protein with other agents.

### Rationale for Dose Selection of FLT3L-Fc Fusion Protein

Selection of the starting dose in this study (675m) is supported by the PK/PD assessment of the published CDX-301 data and the exposure-response relationship for FLT3L-Fc fusion protein in cynomolgus monkeys indicating that the proposed starting dose of FLT3L-Fc fusion protein is predicted to have negligible biological activity (myeloid DC expansion). The starting dose is further supported by the favorable safety of the recombinant FLT3 ligand, CDX-301, in a Phase 1 multiple-dose, dose-ranging study previously conducted in HVs (Anandasabapathy, et al., Bone Marrow Transplant (2015) 50(7):924-30). Based on the current PK/PD-guided predictions and the published dose-response data of CDX-301 and the exposure-response relationship for FLT3L-Fc fusion protein in cynomolgus monkeys (in terms of cDC1 expansion), FLT3L-Fc fusion protein doses of 2000m may induce cDC1 expansion at therapeutically relevant levels and doses of up to 20,000m administered every 4 weeks may help sustain the peak expansion of cDC1 before returning to the baseline. A 4-week dosing interval may be suitable for testing the concept of clinical efficacy in patients with solid tumors.

The nonclinical safety profile of FLT3L-Fc fusion protein has been characterized in a 4-week GLP IV repeatdose toxicity study with 4-week recovery period in cynomolgus monkeys. FLT3L-Fc fusion protein was well tolerated when given IV at dosages of up to 10 mg/kg/week for 4 weeks. The no observed adverse effect level (NOAEL) in the cynomolgus toxicity study was determined to be 10 mg/kg/week, corresponding to an average Cmax of 385 μg/mL and AUCtau of 40,100 μg·h/mL on Day 22.

On a mg/kg basis, the starting dose of 675 μg FLT3L-Fc fusion protein in the present study represents a 900-fold safety margin relative to the NOAEL in cynomolgus monkeys. Based on the current PK projections, FLT3L-Fc fusion protein 675 μg multiple doses are predicted to result in Cmax of 267 ng/mL and AUCtau of 9494 ngh/mL in humans, which correspond to approximately 1440- and 4224-fold safety margins, respectively, relative to the Cmax and AUCtau of the 10 mg/kg NOAEL dose in cynomolgus monkeys.

The proposed starting dose, dose levels, and dosing frequency of FLT3L-Fc fusion protein in the current study may be adjusted based on data from the first-in-human study in healthy volunteers (Example 31). The starting dose of the current study may be adjusted such that any dose level that is found to be well tolerated in healthy volunteers will not be repeated.

The recommended Phase 2 dose (RP2D) to be evaluated in Phase 2 will be selected based on all relevant clinical data from all subjects treated in Phase 1b dose escalation, will consider toxicities through 28 days of dosing, and will not exceed the MTD.

### Risk/Benefit Assessment for the Study

The proposed Phase 1b dose escalation study aims to evaluate FLT3L-Fc fusion protein in patients with advanced malignancies as a single agent to evaluate safety and to determine the MTD. Nonclinical investigations provide sufficient evidence that patients with advanced solid tumors administered FLT3L-Fc fusion protein would not be exposed to unjustifiable risks.

Based on the nonclinical safety profile of FLT3L-Fc fusion protein, the anticipated risks in patients will be related to FLT3 agonism and may include expansion of cDC1 and cDC2 cell populations, peripheral monocytes, lymphocytes, total white blood cells, neutrophils, and eosinophils to a lesser degree. There may also be changes in serum cytokine changes that could reflect the change in dendritic cell populations; however, this is unlikely to lead to acute release of cytokines or cytokine storm which has not been observed in cynomolgus monkeys, the most sensitive species in the nonclinical toxicology studies.

There is postulated risk of developing secondary hematological malignancies with FLT3 activation, based on the observation that aberrant expression of FLT3 is commonly found in hematopoietic malignancies, in particular, acute myeloid leukemia. In most cases, this is due to activating mutations in the FLT3 gene that promote ligand-independent continuous signaling. This theoretical risk has been mitigated by limiting the exposure of patients to FLT3L-Fc fusion protein to 12 months. Moreover, the related recombinant FLT3L (CDX-301) has safely been administered to more than 500 subjects, including over 300 patients for up to 6 months with cancer with no report of secondary leukemia development. There is also theoretical risk of immune related adverse events (irAEs) with agents that modulate the immune system and therefore, any subjects with a history of active autoimmune diseases will be excluded from the study. Some subjects may receive FLT3L-Fc fusion protein alongside SBRT at the discretion of the investigator in line with the local protocol. The recombinant FLT3L (CDX-301) has been administered in combination with SBRT in patients with non-small cell lung cancer; this combination has been well tolerated.

Participants may develop ADAs to FLT3L-Fc fusion protein which may interfere with the physiological actions of endogenous FLT3L. Patients will be monitored for the development of ADAs and any potential sequalae throughout their treatment period with FLT3L-Fc fusion protein and at the end of study.

There may be no direct benefit to subjects participating in this study; however, data from this study will support further development of FLT3L-Fc fusion protein for the treatment of subjects with advanced solid tumors.

Based on available information, the benefit/risk balance for this study is considered justifiable.

### Pharmacokinetic Assessments

### Pharmacokinetic Parameters

FLT3L-Fc fusion protein concentrations will be determined by a validated method. The PK parameters to be estimated and reported may include, but may not be limited to, Cmax, AUCtau, Ctrough, Tmax and CL. Unresolved missing data may be imputed when analysis integrity is affected. The conservative principle will be used for data imputation. Noncompartmental techniques will be used to analyze the PK. Compartmental modeling (eg, population PK) analysis may be conducted.

### PK Sample Collection

Blood sample collection for FLT3L-Fc fusion protein PK characterization will be conducted throughout the study. The time for collection of PK blood draws should always be referenced from the start of the infusion. It is important to record all infusion start dates/times, infusion end dates/times, infusion interruption(s) start and end dates/times, infusion flush end dates/times, and blood sample collection dates/times completely and accurately (and to the nearest minute).

At Cycles 1 and 3, blood will be collected at predose (<30 minutes before start of infusion), end of infusion (+5 minutes), and 2 hours (±10 minutes), 6 hours (±0.5 hours), Day 2 (24 hours [±2 hours]), Day 3 (48 hours [±4 hours]), Day 5 (96 hours [±4 hours]), Day 8 (168 hours [±12 hours]), Day 15 (For Cycle 1: pre-Day 15 dose [<30 minutes before start of infusion], end of Day 15 infusion (+5 minutes), and 2 hours post start of Day 15 infusion (±10 minutes); for Cycle 3: 336 hours [±12 hours]), and Day 24 (552 hours [±12 hours]) post start of the Day 1 infusion (FIG. 30C).

In addition, samples will be collected on Day 1 (predose), and Day 15 (336 hours) of Cycles 2, 4, and every subsequent even numbered cycle thereafter, and at the 60-day follow-up visit (approximately 60 days after last dose). An additional blood sample will be collected at the EOT visit if a subject terminates early from study treatment.

### Biomarker Assessments

### Biomarker Sample Collection

Blood samples will be collected to assess pharmacodynamic responses, immunological response to FLT3L-Fc fusion protein, and correlates of clinical efficacy and/or safety. Samples will include:
Whole Blood for PD Biomarkers (PBMC and Plasma):
   Day 1 of Cycle 1 and Cycle 3 at predose, Day 8 (168 hours [±12 hours]), Day 15 (336 hours [±12 hours], must be collected predose at Cycle 1), and Day 24 (552 hours [±12 hours]) post start of the Day 1 infusion. The predose samples will be collected any time prior to the start of infusion at the Day 1 visit. However, for Cycle 1 Day 1, an additional set of predose samples should be collected up to 72 hours prior to the Cycle 1 Day 1 visit, if feasible.
   Predose on Day 1, and Day 15 (336 hours [±12 hours]) post start of the Day 1 infusion of Cycles 2, 4, and every subsequent even numbered cycle thereafter
   End of treatment (for subjects that terminate early from study treatment only)
   60-day follow-up visit (approximately 60 days after last dose)
   Two 10 ml samples will be collected at all time points except on Days 8 and 24 in Cycles 1 and 3, when only one sample of 10 ml will be collected.
Whole Blood for Leukocyte Count: at the same time points as the PD Biomarkers (PBMC and plasma) samples above
Whole Blood sample for CHIP mutation analysis: at Screening; anytime on Day 1 of Cycles 1, 2, 3, and every subsequent even cycle; and EOT
Whole Blood sample for TCR sequencing: anytime on Day 1 of Cycles 1, 2, 3, and every subsequent even numbered cycle; and EOT
Whole Blood for assessing Circulating Tumor DNA: at Screening; anytime on Day 1 of Cycles 1, 2, 3, and every subsequent even cycle; and EOT
Whole Blood Paxgene RNA: at predose on Day 1 and Day 15 of Cycle 1, predose on Day 1 of Cycle 2, predose on Day 1 of Cycle 3, any time on Day 15 of Cycle 3, and predose on Day 1 at every subsequent even numbered cycle; and EOT
Serum PD for Circulating Factors: at predose on Day 1 of Cycles 1, 2 and 3; Days 8 and 15 of Cycles 1 and 3; Day 24 of Cycle 1; and EOT Stool Sample for Microbiome Sequencing: at predose on Day 1 of Cycles 1
and 3, and EOT. Subjects will collect a sample up to 72 hours prior to the study visit and be reminded to bring in the sample upon their arrival to the clinic.
Whole Blood for Optional Genomic analysis: preferable to collect at predose on Cycle 1 Day 1, but any other time point during the course of the study is also acceptable
Archival tumor tissue biopsy collected at screening for evaluation of tumor mutations, if available.
Optional fresh pre-treatment, on-treatment, and EOT tumor tissue biopsies to assess tumor PD. If archival tumor tissue is not available or insufficient, collection of the optional fresh pre-treatment tumor biopsy is preferred. Optional fresh on-treatment and end of treatment (progressive disease) tumor biopsies will only be collected if a recent pre-treatment tumor biopsy not older than 6 months (recent archival tumor biopsy or optional fresh tumor biopsy) was collected.
Optional biopsies are requested from subjects with accessible disease and should be core needle or excision biopsies.
   The pre-treatment biopsy can be obtained any time after the last line of therapy and prior to the first dose of study drug.
   The on-treatment biopsy can be obtained any time after start of Cycle 2, but strongly preferred between Day 15 of Cycles 2 and 4 after completion of radiographic imaging.
   The EOT biopsy will be collected at progression only for subjects with progressive disease.

### Immunogenicity Assessments

The immunogenicity assessments will be conducted to detect and measure antidrug antibody (ADA) against FLT3L-Fc fusion protein. Subjects will have ADA assessed at predose (<30 minutes before start of infusion) on Day 1 of Cycle 1, 2, 3, 4, 7, and 13; and at 60-day follow-up visit (60 days after last dose). An additional blood sample will be collected at the EOT visit if a subject terminates early from study treatment.

### Efficacy Assessments

### Response Assessment

Response assessment will be performed according to RECIST 1.1 (Eisenhauer, et al., Eur J Cancer (2009) 45(2):228-47).

For all subjects, tumor response assessment will be performed by CT scan with contrast or magnetic resonance imaging (MRI) of the chest/abdomen/pelvis (plus other regions as required for specific tumor types). All scans performed at baseline and other imaging performed as clinically required (other supportive imaging) will be repeated at subsequent visits. In general, lesions detected at baseline should be followed using the same imaging methodology and preferably the same imaging equipment at subsequent tumor evaluation visits.

For each subject, the investigator will designate 1 or more of the following measures of tumor status to follow for determining response: CT or MRI images of primary and/or metastatic tumor masses, physical examination findings, and results of other assessments. All available images collected during the study period will be considered. The most appropriate measures to evaluate a subject's tumor status should be used. Measure(s) chosen for sequential evaluation during the study must correspond to measures used to document progressive tumor status that qualifies the subject for enrollment.

Subjects who experience initial radiologic progressive disease and are doing well clinically are considered to have initial RECIST 1.1-defined progressive disease and will be permitted, with sponsor's approval, to continue with study drug treatment. These subjects will be reevaluated using the same imaging modality no less than 4 weeks later (after the last imaging with initial RECIST 1.1-defined progressive disease) to assess whether study drug treatment will be continued. If initial progression is based on occurrence of a new lesion in an area not scanned at baseline, an on-study scan no less than 4 weeks from initial observation of new lesion should be considered before performing the end of treatment visit.

Tumor responses to treatment will be assigned based on evaluation of response of target, nontarget, and new lesions according to RECIST 1.1 (all measurements should be recorded in metric notation; see Eisenhauer, et al., Eur J Cancer (2009) 45(2):228-47. To assess objective response, tumor burden at baseline will be estimated and used for comparison with subsequent measurements. At baseline, tumor lesions will be categorized in target and non-target lesions as described in Eisenhauer, et al., Eur J Cancer (2009) 45(2):228-47.

Results for these evaluations will be recorded with as much specificity as possible so that pretreatment and post-treatment results will provide the best opportunity for evaluating tumor response.

Any complete response (CR) or partial response (PR) should be confirmed by CT or MRI scan as described in Eisenhauer, et al., Eur J Cancer (2009) 45(2):228-47 no less than 4 weeks after initial assessment.

The investigator may perform scans in addition to a scheduled study scan for medical reasons or if progressive disease is suspected.

Subjects who may receive SBRT at investigator's decision will not have their designated target lesion (per RECIST 1.1) subjected to SBRT.

### Tumor Assessments

### Tissue Collection

Tumor tissue for biomarker analysis will be collected at the time of screening from an archival tumor biopsy, obtained preferably either at the time of or after the diagnosis of advanced disease has been made, and from a site not previously irradiated.

If a tumor biopsy was obtained from a target lesion during eligibility assessment, it is preferred that a new baseline scan be obtained.

Additional tissue may be collected for subjects who consent to the optional fresh tumor tissue biopsies.

If a subject signs the consent for the optional fresh tumor biopsies, one will be collected pre-treatment (if archival tumor tissue is not available), on-treatment any time after the start of Cycle 2, but strongly preferred between Day 15 of Cycles 2 and 4 after completion of radiographic imaging, and at the time of progressive disease, if clinically applicable and feasible. These biopsies will be used for exploratory assessment of pharmacodynamics.

Optional fresh on-treatment and end of treatment (progressive disease) tumor biopsies will only be collected if a pre-treatment tumor biopsy (archival or fresh) was collected.

For additional details and instructions regarding tissue requirements, collection, storage, and shipment, refer to the study laboratory manual.

### Tumor Imaging

Investigator-assessed imaging will be performed at defined time points. The initial tumor imaging to establish baseline disease will be performed ≤28 days prior to first dose of study drug. Scans performed as part of routine clinical management are acceptable for use as screening scan if they are of diagnostic quality and ≤28 days prior to first dose of study drug. On-study imaging as listed in Table 35 will be performed as specified below and in FIG. 30D.

TABLE 35
Postbaseline Imaging Schedule

| | Postbaseline Imaging | Duration of Tumor Imaging |
|---|---|---|
| Cohort | Schedule | Assessments |
| All | Every 8 weeks from the | Until progressive disease is assessed |
| cohorts | first treatment dose | by investigator, new line of |
| | (Visit window ± 7 days) | anticancer therapy, or 1 year after |
| | | last treatment dose, whichever occurs |
| | | first |

The timing of on-study treatment imaging should follow study calendar days starting from Cycle 1 Day 1 and should not be adjusted for delays in treatment administration or for visits. The same imaging technique should be used in a subject throughout the study. In general, lesions detected at baseline should be followed using the same imaging methodology and preferably the same imaging equipment at subsequent tumor evaluation visits. The investigator may perform scans in addition to a scheduled study scan for medical reasons or if progressive disease is suspected.

For subjects who permanently discontinued all study drug(s) in the absence of progressive disease (e.g., experienced unexpected toxicity) and/or start of new anticancer therapy, CT or MRI imaging should continue to be performed at the predefined schedule, until documented progressive disease, initiation of a new anticancer therapy other than the study treatment, or up to 1 year after the last dose of study drug, whichever occurs first; and then move into survival follow-up for up to 1 year after the completion/discontinuation of tumor lesion assessments.

For subjects who permanently discontinue from the study in the absence of progressive disease/or start of a new line of anticancer therapy and will not be continuing tumor imaging during the posttreatment period (ie, withdrew consent), additional imaging is recommended at the EOT visit if the last imaging was performed more than 30 days prior.

For subjects that met progressive disease clinically, subjects will continue tumor imaging until progressive disease is confirmed radiographically or prior to the start of a new line of anticancer therapy.

### Treatment Assessments

### Baseline/Cycle 1 Day 1 Predose Assessments

The following will be performed and documented prior to study treatment administration:
Review inclusion and exclusion criteria
Review baseline symptoms
Focused physical examination (may be performed within 72 hours prior to the first dose of study treatment)
Vital signs (temperature, pulse, respiratory rate, and blood pressure)
Weight
Standard 12-lead ECG
Assess ECOG performance status (may be performed within 72 hours prior to the first dose of study treatment)
Obtain predose blood samples for:
   Hematology and serum chemistry (may be performed within 72 hours prior to the first dose of study treatment)
   FLT3L-Fc fusion protein PK (<30 minutes before start of infusion)
   Immunogenicity assessment (<30 minutes before start of infusion)
   Whole blood for PD biomarkers (PBMC and plasma). The predose sample will be collected any time prior to the start of infusion at the Day 1 visit. An additional set of predose samples should be collected up to 72 hours prior to the Cycle 1 Day 1 visit, if feasible.
   Whole blood for leukocyte count. The predose sample will be collected any time prior to the start of infusion at the Day 1 visit. An additional set of predose samples should be collected up to 72 hours prior to the Cycle 1 Day 1 visit, if feasible.
   Whole blood for paxgene RNA
   Serum PD for circulating factors
   Whole blood for optional genomic research (for subjects that provide additional consent)
Obtain predose urine samples for:
   Urinalysis (may be performed within 72 hours prior to the first dose of study treatment)
   Urine pregnancy test for females of childbearing potential (may be performed within 72 hours prior to the first dose of study treatment)
Obtain predose stool sample for microbiome sequencing. Subjects will collect a sample up to 72 hours prior to the study visit and be reminded to bring in the sample upon their arrival to the clinic.
Review and record all AEs and concomitant medications

Following the completion of the above assessments, the subject will be enrolled and administered study drug.

### Cycle 1 Day 1 Postdose Assessments

The following will be performed and documented after administration of study treatment:
Obtain postdose blood samples for:
   FLT3L-Fc fusion protein PK (at end of infusion [+5 minutes], 2 hours [±10 minutes], and 6 hours [±0.5 hours] post start of infusion)
   Whole blood for CHIP mutation analysis
   Whole blood for TCR sequencing
   Whole blood for circulating tumor DNA
Vital signs (temperature, pulse, respiratory rate, and blood pressure; 1 hour [+15 minutes] after end of infusion)
Standard 12-lead ECG (2 hours [−10/+20 minutes] after end of infusion)
Observation for 1 hour after end of infusion for irAEs

### Clinical Laboratory Assessments

The central laboratory will be responsible for chemistry, hematology, coagulation, endocrine function, urinalysis, HIV, HBV and HCV serology, and serum pregnancy testing as well as processing and/or storage of other study samples. Specific instructions for processing, labeling, and shipping samples will be provided in a central laboratory manual. The date and time of sample collection will be reported to the central laboratory.

If central laboratory results are not available, local laboratories may be used for dosing decisions. Local laboratory assessments resulting in a dose change or as part of an AE assessment, which is not supported by central laboratory results, will be reported on the eCRF. Gilead's standard reference ranges will be used.

Urine pregnancy tests will be performed locally at the site as shown in FIG. 30D. Following the 60-day follow-up visit, urine pregnancy tests will be performed monthly until 12 weeks after the last dose of study drug.

Laboratory tests for screening should be performed within 28 days prior to the first dose of study treatment. Results must be reviewed by the investigator or qualified designee and found to be acceptable prior to the first dose of study treatment. The report of the results must be retained as a part of the subject's medical record or source documents. Blood samples for study-related tests will be collected at time points specified in FIG. 30D.

### Planned Analyses

### Interim Analysis

### Dose-Escalation Analysis

For the purpose of making the decision to escalate to the next dose level/cohort, interim analyses of relevant safety and available PK data will be conducted by sponsor after all subjects in each cohort have completed dosing and the follow-up period in the DLT period as defined in the Dose Escalation section. Safety assessments (eg, AEs, ECG, and laboratory results) will be displayed by cohort to facilitate the decision to dose escalate.

### Final Analysis

The final analysis will be performed after all subjects have completed or discontinued from the study, outstanding data queries have been resolved or adjudicated as unresolvable, and the data have been cleaned and finalized.

### Analysis Conventions

### Analysis Sets

### All Enrolled Analysis Set

The All Enrolled Analysis Set includes all subjects who received a study subject identification number. This will be the primary analysis set for by-subject listings.

### DLT Analysis Set

The DLT Analysis Set includes all subjects who were enrolled for dose escalation, received all treatments of FLT3L-Fc fusion protein and completed safety procedures through Day 28 (inclusive) or experienced a DLT prior to Day 28. Determination of MTD or RP2D will be based on the DLT Evaluable Analysis Set.

### Safety Analysis Set

The Safety Analysis Set will include all subjects who received at least 1 dose of FLT3L-Fc fusion protein. This will be the primary analysis set for safety analysis.

### Full Analysis Set

The Full Analysis Set includes all enrolled subjects who received at least 1 dose of FLT3L-Fc fusion protein. This will be the primary analysis set for efficacy analyses.

### Pharmacokinetics (PK) Analysis Set

The PK Analysis Set includes all enrolled subjects who received at least 1 dose of FLT3L-Fc fusion protein and have at least 1 non-missing postdose concentration value reported by the PK laboratory. This will be the primary analysis set for all PK analyses.

### Immunogenicity Analysis Set

The Immunogenicity Analysis Set includes all enrolled subjects who received at least 1 dose of FLT3L-Fc fusion protein and had at least 1 ADA test. This will be the primary analysis set for immunogenicity data analyses.

### Biomarker Analysis Set

The Biomarker Analysis Set includes all enrolled subjects who received at least 1 dose of FLT3L-Fc fusion protein and have at least 1 evaluable biomarker measurement available. This will be the primary analysis set for all biomarker data analyses.

### Demographic and Baseline Characteristics Analysis

Demographic and baseline measurements will be summarized using standard descriptive methods. Demographic summaries will include sex, race/ethnicity, and age. Baseline data will include a summary of body weight, height, and body mass index.

### Efficacy Analysis

The ORR and the corresponding 90% CI based on the Clopper-Pearson exact method will be provided. Subjects who do not have sufficient baseline or on-study tumor assessment to characterize response will be counted as non-responders.

PFS, DOR, and OS will be analyzed using Kaplan-Meier (KM) method. The median, 25% and 75% percentiles will be provided along with the corresponding 90% CI. In addition, the estimated rate at selected time point, such as 3 months, 6 months, 12 months, 18 months, and 24 months will be reported. The detailed censoring rules will be described in the statistical analysis plan.

TTR will be summarized using descriptive statistics.

### Safety Analysis

All safety data collected on or after the date that study drug was first administered up to the last dose date plus 60 days will be summarized by dose level and/or treatment (according to the study drug received) using the Safety Analysis Set.

For categorical safety data including incidence of AEs and categorizations of laboratory data, count and percentage of subjects will be summarized. For continuous safety data including laboratory data, descriptive summary statistics (mean, standard deviation, minimum, quartiles, median and maximum) will be summarized.

### Pharmacokinetic Analysis

Serum concentrations for FLT3L-Fc fusion protein will be summarized by nominal sampling time using descriptive statistics (ie, sample size, arithmetic mean, geometric mean, % coefficient of variation, standard deviation, median, minimum, and maximum) by dose level/cohort. Serum concentrations of FLT3L-Fc fusion protein over time may be plotted in semi-logarithmic and linear formats as mean±standard deviation by dose level/cohort.

Pharmacokinetic parameters (AUCtau, Cmax, Ctrough, Tmax, CL, etc, as appropriate) will be listed and summarized using descriptive statistics by dose level/cohort.

### Biomarker Analysis

Pharmacodynamics data will be listed and summarized using descriptive statistics by treatment and population. Descriptive summaries for change from baseline will also be provided over time by dose level. Correlation of PD responses, immunologic changes with FLT3L-Fc fusion protein treatment, and other biomarkers with clinical response and/or safety may be explored as appropriate.

### Immunogenicity Analysis

Immunogenicity to FLT3L-Fc fusion protein will be evaluated based upon the incidence of ADA formation. Number and percentage of positive or negative ADA results at each specified time point will be summarized by dose level/cohort using the Immunogenicity Analysis Set. Supporting data including treatment, nominal sampling day, actual date and time of sampling, and ADA results, will be included in a listing.

### Description and Handling of FLT3L-Fc Fusion Protein

FLT3L-Fc fusion protein is a lyophilized powder for reconstitution that is formulated with histidine, sucrose, and polysorbate 80 for stabilization. Each vial is manufactured to contain 10 mg of lyophilized drug product to be reconstituted with sterile water for injection. The quantity in each vial ensures a minimum total volume of 5.0 mL can be withdrawn after reconstitution per instructions. The solution will have a pH of 5.9 after reconstitution

### Results

Nine patients were enrolled in 3 dose escalation cohorts, corresponding to cohorts 2-4. Baseline demographic and diseases characteristics for the nine patients are shown in Table 36. Median (range) age was 71 (44-79); 4 (44%) patients were male. Tumor types were pancreatic (n=3), ovarian (n=4), and rectal (n=2).

TABLE 36
Baseline Demographic and Disease Characteristics

| Characteristic | N = 9 |
|---|---|
| Age (years), median (range) | 71 (44-79) |
| Male/Female, n (%) | 4 (44.4)/5 (55.6) |
| ECOG performance status, n (1%) | |
| 0 | 1 (11.1) |
| 1 | 8 (88.9) |
| Solid tumor type, n (%) | |
| Pancreatic | 3 (33.3) |
| Ovarian* | 4 (44.4) |
| Rectal | 2 (22.2) |

*Two ovarian cancer patients identified after Dec. 3, 2021, the data cut-off date
ECOG = Eastern Cooperative Oncology Group

Treatment-related treatment-emergent adverse events (TEAEs) by grade are shown in Table 37. No DLTs or discontinuation due to adverse events (AE) have been observed. DLTs were evaluated during the first 28 days. Three patients had Grade≥3 AEs which were recorded as serious AEs, none of which were considered related to administration of FLT3L-FC fusion protein (SEQ ID NO: 14).

TABLE 37
Treatment-Related Treatment-Emergent Adverse Events
(TEAEs) by Grade

| | Dose | Dose | Dose | |
|---|---|---|---|---|
| | Level 1 | Level 2 | Level 3 | |
| | 2 mg | 6 mg | 12 mg | Total |
| TEAE | (N = 3) | (N = 3) | (N = 3) | (N = 9) |
| Bone pain, n (%) | 0 | 2 (66.7) | 0 | 2 (22.2) |
| Grade 1 | 0 | 2 (66.7) | 0 | 2 (22.2) |
| Fatigue, n (%) | 1 (33.3) | 1 (33.3) | 0 | 2 (22.2) |
| Grade 1 | 1 (33.3) | 1 (33.3) | 0 | 2 (22.2) |
| Tumor pain, n (%) | 1 (33.3) | 1 (33.3) | 0 | 2 (22.2) |
| Grade 2 | 1 (33.3) | 1 (33.3) | 0 | 2 (22.2) |
| Abdominal pain, n (%) | 0 | 1 (33.3) | 0 | 1 (11.1) |
| Grade 1 | 0 | 1 (33.3) | 0 | 1 (11.1) |
| Diarrhea, n (%) | 0 | 1 (33.3) | 0 | 1 (11.1) |
| Grade 2 | 0 | 1 (33.3) | 0 | 1 (11.1) |
| Nausea, n (%) | 0 | 1 (33.3) | 0 | 1 (11.1) |
| Grade 2 | 0 | 1 (33.3) | 0 | 1 (11.1) |
| Night sweats, n (%) | 0 | 0 | 1 (33.3) | 1 (11.1) |
| Grade 1 | 0 | 0 | 1 (33.3) | 1 (11.1) |
| Pruritus, n (%) | 1 (33.3) | 0 | 0 | 1 (11.1) |
| Grade 1 | 1 (33.3) | 0 | 0 | 1 (11.1) |

No subjects experienced any Grade 3 or higher treatment-related TEAS

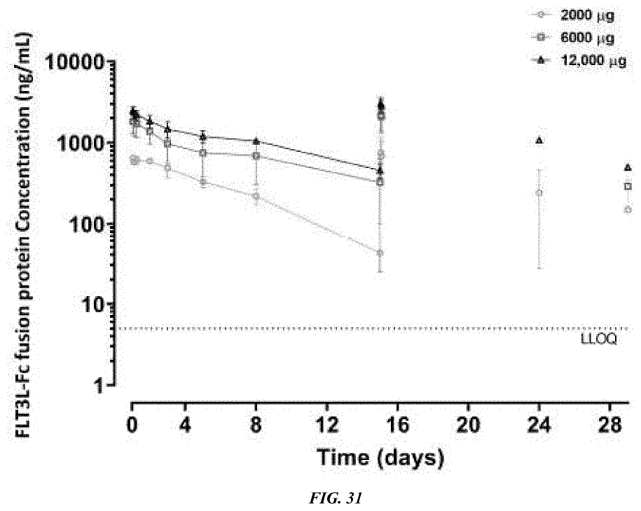

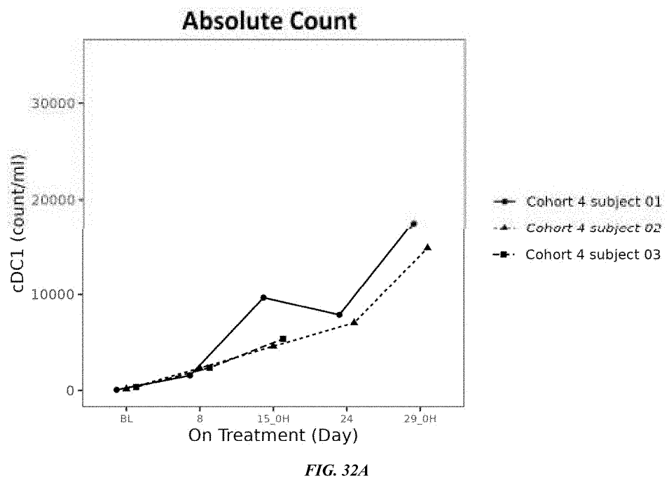

Dose dependent increase in FLT3L-FC fusion protein exposure was observed in the evaluated dose range 2 to 12 mg with target-mediated drug disposition appearing to be saturated at doses above the dose level for Cohort 3 (e.g., 6 mg) Dose Level 2. FLT3L-FC fusion protein accumulation was observed at higher dose levels (FIG. 31). Summary of FLT3L-Fc fusion protein serum pharmacokinetics parameters is shown in Table 38.

TABLE 38
Summary of FTL3L-Fc Fusion Protein Serum PK Parameters

| | Dose Level 1 | Dose Level 2 | Dose Level 3 |
|---|---|---|---|
| PK Parameter | 2 mg (N = 3) | 6 mg (N = 3) | 12 mg (N = 3) |
| Cₘₐₓ (μg/mL), mean (% CV) | 0.643 (6) | 1.86 (30) | 2.52 (10) |
| Tₘₐₓ (h), median (range) | 1 (1-1) | 2 (1-6) | 1 (1-2) |
| C_{trough} (μg/mL), mean | 0.0425 (134) | 0.324 (92) | 0.457 (20) |
| (% CV) | | | |
| AUC₀₋₁₅ (μg*h/mL), mean | 82.2 (24) | 248 (50) | 352 (12) |
| (% CV) | | | |

AUC₀₋₁₅ = area under the plasma concentration vs. time curve from 0 to 15;
Cₘₐₓ = maximum concentration;
C_{trough} = trough plasma concentration;
CV = coefficient of variation;
Tₘₐₓ = time to maximum concentration.

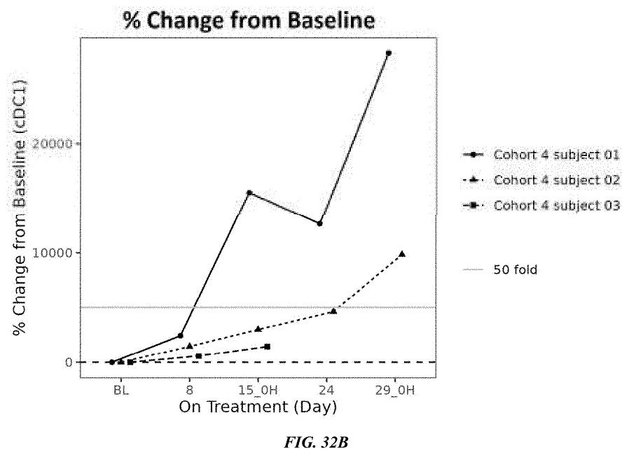

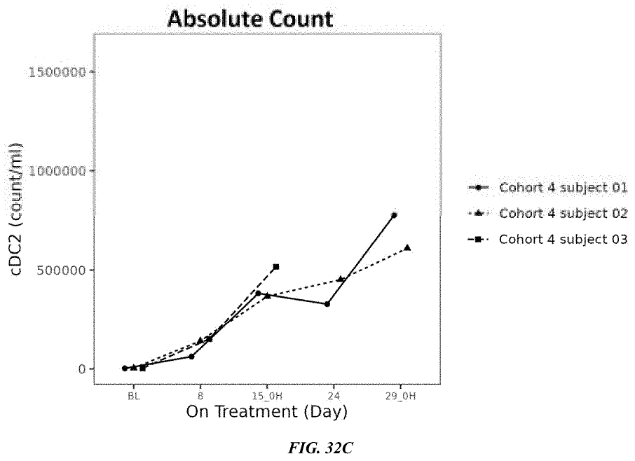

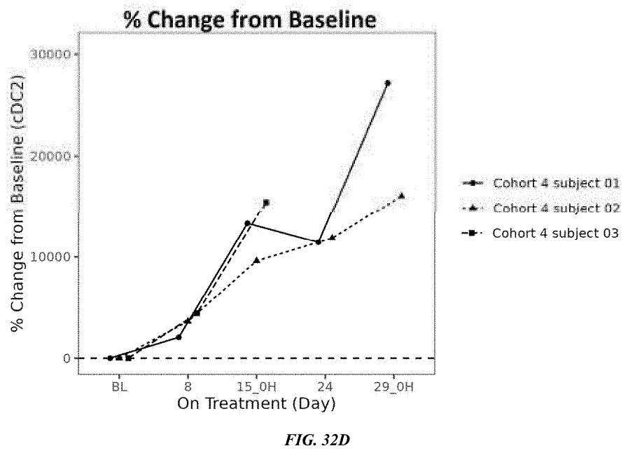

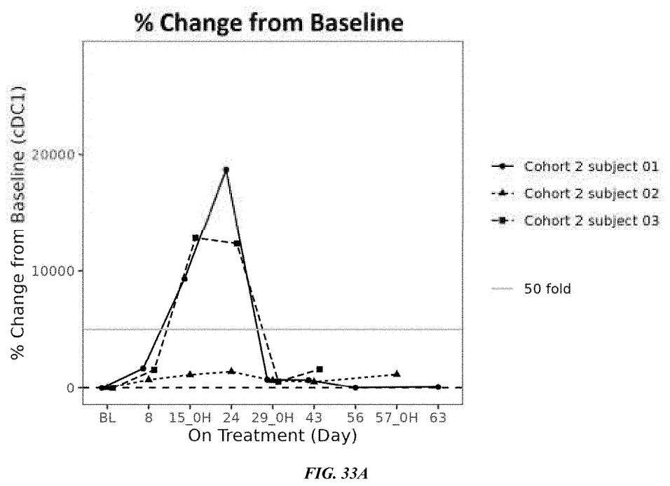

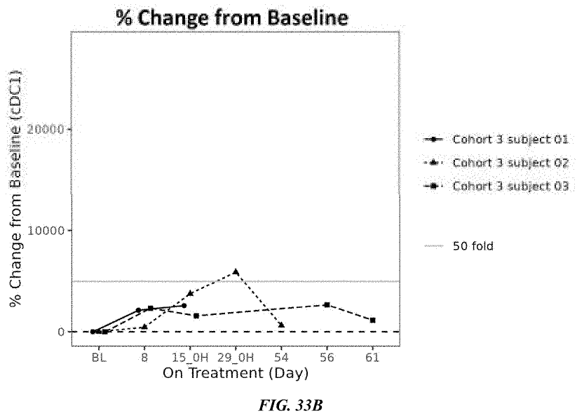

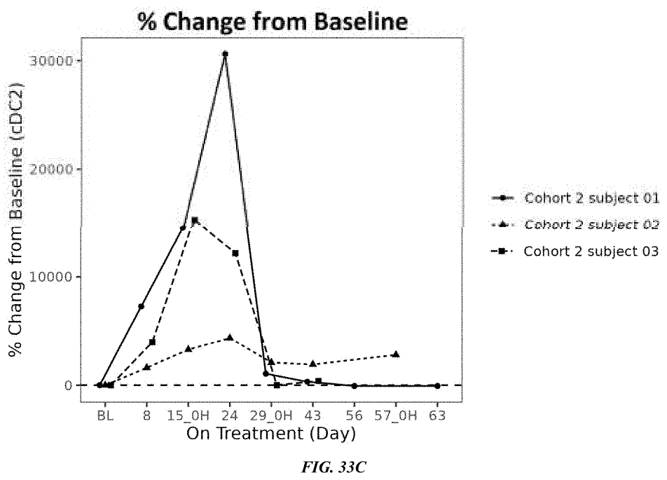

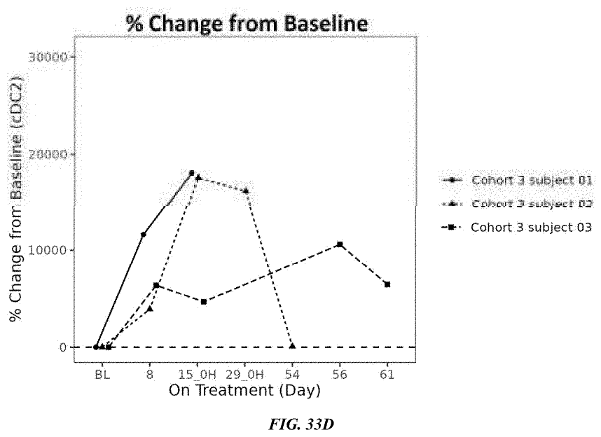

FLT3L-Fc fusion protein treatment resulted in expansions of cDC1 and cDC2 at all 3 doses (Table 39); a dose-dependent trend in the magnitude and the durability of cDC expansion was observed. cDC1 and cDC2 in all 3 subjects of the 12-mg dose level showed continuous increases over time with repeated dosing up to C2D1 (FIGS. 32A-D). Two of the 3 subjects in the 12-mg dose level had >50-fold increases in cDC1 cells by C2D1. cDC1 and cDC2 expansions were higher in magnitude and more durable with increasing dose levels (FIGS. 32B and D and 33A-D). At the highest dose evaluated, FLT3L-Fc fusion protein produced ≥100-fold expansion of both cDC1 and cDC2 at multiple time points. Dose escalation on the study is still ongoing.

TABLE 39

| | Cohort (mg FLT3L-FC fusion protein) | | |
|---|---|---|---|
| | 2 (2 mg) | 3 (6 mg) | 4 (12 mg) |
| Median peak cDC1 fold | 124 (188-15) | 83 (0) | 191 (283-99) |
| expansion (range) | | | |
| Observation of peak cDC1 | C1D24 | C2D15 | C2D1 |
| expansion | | | |
| Median peak cDC2 fold | 147 (154-34) | 201 (0) | 217 (273-162) |
| expansion (range) | | | |
| Observation of peak cDC2 | C1D15 | C2D15 | C2D1 |
| expansion | | | |

### Example 33

### A Phase 2 Study Evaluating the Safety and Efficacy of Novel Treatment Combinations in Patients with Lung Cancer: Patients with Disease Progression after Receiving Systemic Treatment for Non-Small Cell Lung Carcinoma

This study evaluates the efficacy and safety of a combination treatment regimen comprising sacituzumab govitecan and a FLT3L-Fc fusion protein for participants with advanced non-small cell lung carcinoma (NSCLC) or metastatic NSCLC (mNSCLC) with progression on or recurrence after platinum-based chemotherapy and PD-1/PD-L1 immunotherapy, received either in combination or sequentially. Participants with EGFR, ALK, or any other known actionable genomic alterations must have also received treatment with at least 1 approved tyrosine kinase inhibitor appropriate to the genomic alteration.

### Study Design Overview

This is a Phase 2, open-label, multicenter, randomized, controlled study to evaluate novel treatment combinations in patients with lung cancer, specifically, participants with advanced NSCLC or mNSCLC who progressed on or after receiving platinum-based chemotherapy in combination or sequentially with anti-PD-1/PD-L1 therapy. This study consists of a preliminary stage and an expansion stage. In the preliminary stage, an experimental treatment arm (e.g., combination treatment arm comprising sacituzumab govitecan and a FLT3L-Fc fusion protein (SEQ ID NO: 14)) is compared to the historical standard of care benchmark and not directly to each other within the study. In the expansion stage, an investigational treatment arm (e.g., combination treatment arm) is compared to the comparator arm (e.g., sacituzumab govitecan only) of the study. During the preliminary stage, approximately 23 participants are enrolled in the treatment arm. The decision to proceed with the expansion stage is based on the results of clinical efficacy, safety, and tolerability data analysis, as well as the overall landscape at the time of expansion. The ultimate decision to proceed to expansion stage is at sponsor's discretion. If the treatment arm has minimal clinical activity or unacceptable toxicity, the study will not proceed to the expansion stage. Additional participants may be enrolled to ensure adequate enrollment in the treatment arm with respect to study demographics and participant characteristics, such as predictive biomarkers, to facilitate subgroup analyses. Participants receive study treatment until progressive disease as assessed by the investigator, unacceptable toxicity, death, or another treatment discontinuation criterion is met.

### Objectives

The primary objective is to evaluate the objective response rate (ORR) assessed per RECIST version 1.1.

The secondary objectives are (a) to evaluate the efficacy of the treatment combination; and (b) to evaluate the safety and tolerability of the treatment combination.

Exploratory objectives are (a) to evaluate biomarkers in blood and tumor biopsy samples, as applicable to treatment; (b) to explore biomarkers that may predict response/resistance to therapy; and (c) to characterize the pharmacokinetics and immunogenicity, as applicable.

### Endpoints

The primary endpoint is the objective response rate (ORR), defined as the proportion of participants achieving a complete response (CR) or partial response (PR). CR and PR require confirmation at least 4 weeks after the first detection of response and as assessed by the investigator according to RECIST version 1.1.

The secondary endpoints are:
Progression-free survival (PFS), defined as the time from the date of randomization until disease progression (PD), as assessed by the investigator according to RECIST version 1.1 or death, whichever comes first.
Duration of response (DOR), defined as the time from the first response (CR or PR) until the first documented PD, as assessed by the investigator according to RECIST version 1.1 or death, whichever comes first.
Overall survival (OS), defined as the time from the date of randomization until death from any cause.
The incidence of treatment-emergent adverse events (TEAEs), treatment-related adverse events and laboratory abnormalities.

Exploratory endpoints are:
Change in biomarkers in response to treatment
Correlation of clinical response with biomarkers at baseline and/or on treatment/progression
Peak and trough concentrations over time and antidrug antibodies over time

### Dosing and Administration

During the preliminary stage, participants are administered a combination of a FLT3L-Fc fusion protein (SEQ ID NO: 14) and sacituzumab govitecan (e.g., combination arm). During the expansion stage, participants are administered sacituzumab govitecan (e.g., comparator arm) or a combination of a FLT3L-Fc fusion protein (SEQ ID NO: 14) and sacituzumab govitecan (e.g., combination arm). For the comparator arm, patients are infused with sacituzumab govitecan at a dose of 10 mg/kg. For the combination arm, participants are administered FLT3L-Fc fusion protein intravenously over 60 (±10) minutes at a dose of 12,000 μg, followed by a 30-60-minute observation period, followed by an infusion of sacituzumab govitecan at a dose of 10 mg/kg. For both arms, the first infusion of sacituzumab govitecan on cycle 1, day 1 (C1D1) is over 3 hours. For both arms, subsequent infusions of sacituzumab govitecan may be administered over 60-120 (±5) minutes followed by a 30-minute observation period. For the comparator arm, sacituzumab is administered on days 1 and 8. For the combination arm, FLT3L-Fc fusion protein is administered on day 1 and sacituzumab is administered on days 1 and 8. Treatment with FLT3L-Fc fusion protein may continue for up to 8 treatment cycles. For both arms, Participants may be infused with sacituzumab govetican until PD or unacceptable toxicity.

### Sacituzumab Govitecan Dose Modifications and Treatment Delays

The major toxicities of sacituzumab govitecan are expected to be GI symptoms and neutropenia. Table 37 summarizes recommendations for sacituzumab govitecan dose reductions and discontinuations for treatment-related toxicities.

Sacituzumab govitecan dose reductions and interruptions are managed based on toxicity severity. Leukopenia or lymphopenia in the absence of neutropenia does not require dose modification. The sacituzumab govitecan dose must not be re-escalated following a dose reduction. The sacituzumab govitecan treatment must be discontinued if there is a 3-week dose delay from the planned treatment date due to treatment-related toxicity or 5-week dose delay for all other reasons.

In cases where the toxicity is attributed to the combination partner only, reinitiation of sacituzumab govitecan as a monotherapy may be considered after communication with and agreement by the sponsor.

TABLE 37
Recommended Dose Modification Schedule for Sacituzumab Govitecan

| | | |
|---|---|---|
| Adverse Reaction | Occurrence | Dose Modification or Action |

Severe neutropenia

| | | |
|---|---|---|
| Grade 4 neutropenia ≥ 7 days, | First | Reduce dose to 7.5 mg/kg |
| OR | | and |
| Grade 3 or 4 febrile neutropenia | | administer G-CSF |
| OR | Second | Reduce dose to 5 mg/kg and |
| At time of scheduled treatment, Grade 3 or 4 | | administer G-CSF |
| neutropenia that delays dosing by 2 or 3 weeks | Third | Discontinue Treatment |
| for recovery to ≤ Grade 1 | | |
| At time of scheduled treatment, Grade 3 or 4 | First | Discontinue Treatment |
| neutropenia that delays dosing beyond 3 weeks | | |
| for recovery to ≤ Grade 1 | | |

Severe nonneutropenic toxicity

| | | |
|---|---|---|
| Grade 4 nonhematologic toxicity of any | First | Reduce dose to 7.5 mg/kg |
| duration, | Second | Reduce dose to 5 mg/kg |
| OR | Third | Discontinue treatment |
| Any Grade 3 or 4 nausea, vomiting, or diarrheaa | | |
| due to treatment that is not controlled with | | |
| antiemetics and antidiarrheal agents | | |
| OR | | |
| Other Grade 3 or 4 nonhematologic toxicity | | |
| persisting > 48 hours despite optimal medical | | |
| management, | | |
| OR | | |
| At time of scheduled treatment, Grade 3 or 4 | | |
| nonneutropenic hematologic or | | |
| nonhematologic toxicity that delays dose by 2 | | |
| or 3 weeks for recovery to ≤ Grade 1 | | |
| In the event of Grade 3 or 4 nonneutropenic | First | Discontinue treatment |
| hematologic or nonhematologic toxicity that | | |
| does not recover to ≤ Grade 1 within 3 weeks | | |

Infusion-Related Toxicities

| | | |
|---|---|---|
| Grade 2 or Grade 3 infusion-related reactions | Recurrent | Discontinue treatment |
| despite optimal management | | |
| Grade 4 infusion-related reaction | First | Discontinue treatment |

G-CSF = granulocyte colony-stimulating factor;
NCI-CTCAE = National Cancer Institute-Common Terminology Criteria for Adverse Events
The severity of AEs are graded using the Common Terminology Criteria for Adverse Events Version 5.
a For Grade 1-2 treatment-related diarrhea, administer loperamide at an initial dose of 4 mg, followed by 2 mg with every episode of diarrhea to a maximum dose of 16 mg/day. If diarrhea is not resolved after 24 hours, consider adding diphenoxylate/atropine. Consider adding octreotide 100 to 150 mcg subcutaneous 3 times per day if diarrhea persists.

Sacituzumab govitecan is administered in 21-day cycles on Days 1 and 8; the next cycle should start 14 days after the Day 8 dose (i.e., the Day 8 infusion is counted as the first day of that 14-day period). However, visit windows of 1 day prior to and 2 days after the scheduled infusion are permitted. The scheduled Day 1 and Day 8 infusions may be delayed for up to 3 weeks for treatment-related toxicities.

Instructions for dose delays and dose reductions for specific toxicities are summarized below. See Table 38 for when sacituzumab govitecan can be administered based on ANC. Withhold sacituzumab govitecan administration for Grade 3 nausea or Grade 3 or 4 diarrhea or vomiting at the time of scheduled treatment administration and resume sacituzumab govitecan when resolved to ≤Grade 1. For other toxicities, dosing may be delayed for >Grade 2 toxicities for a maximum of 3 weeks per investigator assessment. If the toxicity has improved to ≤Grade 2 the dose should be administered at that time. For a toxicity that delays Day 8 dosing, if dosing is delayed for more than 1 week, dosing should resume on Day 1 of next cycle to minimize treatment gap. Regardless of whether the Day 8 dose is delayed for toxicity, there should be 14 days between the Day 8 infusion and the Day 1 infusion of the next cycle.

TABLE 38
Guidance for Premedication and Prophylaxis for Toxicities Associated with
Sacituzumab Govitecan

| Potential Reaction | Premedication and Prophylaxis Guidance |
|---|---|
| Infusion-related | Antipyretics and H1/H2 blockers should be administered before |
| reaction | each sacituzumab govitecan infusion |
| | Corticosteroids (hydrocortisone 50 mg or equivalent PO or IV) |
| | may be administered prior to infusions. |
| Nausea and vomiting | Premedication with a 2-drug antiemetic regimen is |
| | recommended. |
| | If nausea and vomiting are persistent, a 3-drug regimen may be |
| | used, including a 5-HT3 inhibitor (ondansetron or |
| | palonosetron, or other agents according to local practices), an |
| | NK1-receptor antagonist (fosaprepitant or aprepitant), and |
| | dexamethasone (10 mg PO or IV). |
| | Anticipatory nausea can be treated with olanzapine. |
| Neutropenia | Complete blood counts must be obtained prior to each |
| | sacituzumab govitecan infusion and treatment should be |
| | administered if ANC meets the following criteria: |
| | Day 1: ANC ≥ 1500/mm3 |
| | Day 8: ANC ≥ 1000/mm3 |
| | The routine prophylactic use of growth factors is not required; |
| | however, prophylactic administration should comply with |
| | current ASCO/ESMO guidelines for use of growth factors. |

ANC = absolute neutrophil count;
ASCO = American Society of Clinical Oncology;
ESMO = European Society for Medical Oncology,
IV = intravenous;
PO = orally;
SG = sacituzumab govitecan

Palliative radiotherapy is permitted. If there is clear evidence of clinical benefit, treatment may be continued after completion of palliative radiotherapy. In this case, sacituzumab govitecan administration should be interrupted 1 week before the procedure and reinstated no earlier than 2 weeks after the procedure. In the event a patient requires surgery, sacituzumab govitecan should be interrupted 1 week before the procedure if clinically feasible and dosing should be held for 2 weeks after the procedure. Dosing may resume thereafter if the patient is clinically stable. Extensive surgical procedures (eg, abdominal, cranial surgeries) may require suspension of dosing for 4 weeks to allow for an adequate period for healing before dosing may resume. The study medical monitor must approve continuation of therapy with sacituzumab govitecan before resumption of dosing.

Treatment interruptions for reasons other than resolution of toxicities/procedures are not permitted outside of the permitted visit windows.

### Treatment Modifications of FLT3L-Fc Fusion Protein

Treatment modifications of FLT3L-Fc fusion protein, including dose delays, temporary interruptions, or permanent treatment discontinuation, may be required in the event of treatment-related toxicity.

As such, participants randomized to FLT3L-Fc fusion protein who experience toxicity are to withhold or discontinue FLT3L-Fc fusion protein. Dose reductions are not permitted.

In situations in which treatment can be restarted, FLT3L-Fc fusion protein should be restarted concurrently. If the combination has been withheld for more than 3 consecutive doses because of toxicity, the participant should permanently discontinue the combination unless otherwise agreed with the medical monitor.

### Permanent Discontinuation From Treatment Study

Any study treatment may be discontinued in the following instances:
Intercurrent illness that would, in the judgment of the investigator, affect assessments of clinical status to a significant degree.
Unacceptable toxicity, or toxicity that, in the judgment of the investigator, compromises the ability to continue study-specific procedures or is considered not to be in the participant's best interest.
Death.
Disease progression with no evidence of clinical benefit.
Initiation of alternative anticancer therapy, including any study drug. Palliative and/or supportive medication is permitted as detailed herein.
Pregnancy during the study.
Investigator or treating physician decision.
Participant or legally acceptable representative request, with or without a stated reason.
Participant noncompliance with study drugs.
Lost to follow-up.
Discontinuation of the study at the request of sponsor or a regulatory agency/institutional review board (IRB)/independent ethics committee (IEC).
Unforeseen global pandemic or natural disaster.

The reason for treatment discontinuation must be reported.

Study drug discontinuation should not result in participant discontinuation from the study. Participants should continue to be followed for safety, disease progression (if relevant), and survival.

### Discontinuation Criteria

Participants are to discontinue the study under any of the following instances:
Death
Participant withdrawal of consent from the study
Lost to follow-up
Discontinuation of the study at the request of the sponsor or a regulatory agency/IRB/IEC

The reason for study discontinuation must be reported.

### Lost to Follow-Up

A participant is considered lost to follow-up if the participant repeatedly fails to return for scheduled visits and is unable to be contacted by the study site.

The following actions must be taken if a participant fails to return to the clinic for a required study visit:
The site must attempt to contact the participant and reschedule the missed visit as soon a possible, counsel the participant on the importance of maintaining the assigned visit schedule, and ascertain whether the participant wishes to and/or should continue in the study.
Before a participant is deemed lost to follow-up, the investigator or designee must make every effort to regain contact with the participant, with at least 3 attempts by a combination of phone calls, text messages, email messages, and if necessary, a certified letter to the participant's last known mailing address or local equivalent methods. These contact attempts should be documented in the participant's source documents. If a participant does not respond within 1 month after the third contact attempt, the participant is considered lost to follow-up.
For participants who are considered lost to follow-up prior to completion of all protocol-required study assessments or survival follow-up as described in Tables 40 and 41, the investigator may search publicly available records (where permitted by local laws and regulations) to ascertain survival status unless the participant has withdrawn consent for such follow-up. This ensures reduced risk of missing critical efficacy data.

### End of Study

The end of the study is defined as the date on which the last participant in the clinical study completes the last study visit/call or when the sponsor decides to end the study. The sponsor reserves the right to terminate the study at any time for any reason (including safety).

### Adverse Events and Toxicity Management

### Requirements for AE, SAE, and Other Reportable Safety Event Collection

TABLE 39
Time Period for Collecting AE, SAE, and other Reportable Safety Event Information

| Reportable Safety Event | Time Period^{a} |
|---|---|
| All SAEs and any AEs related | After signing informed consent until first dose of study |
| to a protocol-mandated | treatment |
| procedure | |
| All AEs and SAEs regardless | Following first dose of study treatment until 100 days after |
| of causality | last administration of study treatment |
| Pregnancy in a study | Following initiation of study drug until 6 months after last |
| participant or female partner of | administration of study treatment for a female participant or |
| a study participant | 4 months for female partner of a participant |
| Special situations reports^{b} | Following first dose of study treatment until 100 days after |
| | last dose of study treatment |

AE = adverse event;
SAE = serious adverse event
^{a}Investigators are not obligated to actively seek AE or SAE information after the end of the reporting period. However, if the investigator learns of any SAE, including a death, at any time after the end of the reporting period, and he/she considers the event to be reasonably related to study treatment or study participation, the event should be promptly reported to the sponsor or its designee. Every effort should be made to follow AEs and SAEs considered related to study treatment or protocol-related procedures until a final outcome can be reported.
^{b}Includes special situations reports that involve sponsor products including concomitant medications that are not considered study drug.

### Toxicity Management

For Grade 3 and 4 toxicities, relationship to study drugs, clinical status of participant, and investigator assessment of participant safety should inform participant withdrawal from dosing. Abnormal laboratory values should be repeated when necessary and followed until resolution and as clinically appropriate. Treatment-emergent toxicities are noted by the investigator and brought to the attention of the medical monitor, and the appropriate course of action will be discussed and decided. Whether or not considered treatment-related, all participants experiencing AEs must be monitored periodically until symptoms subside, any abnormal laboratory values have resolved or returned to baseline levels or they are considered irreversible, or until there is satisfactory explanation for the changes observed.

### Participant Population

Approximately 23 participants with advanced or metastic NSCLC are enrolled in the treatment arm (e.g., combination arm) during the preliminary stage of this study. If the combination treatment is demonstrating efficacy, the study may proceed to the expansion stage, in which 55 participants with advanced or metastic NSCLC are enrolled in the treatment arm (e.g., combination arm). The expansion stage will also include

### Inclusion Criteria

Subjects must meet all of the following inclusion criteria to be eligible for participation in this study:
1) Histologically or cytologically documented NSCLC with documented evidence of stage IV NSCLC disease at time of start of study treatment (based on the American Joint Committee on Cancer, Eighth Edition).
2) Participants willing to provide adequate tumor tissue as specified in the Laboratory Manual, ideally a new biopsy. Tumor biopsy must have been performed on or after progression on prior line of therapy and before enrollment with no anticancer treatment between collection of tissue and enrollment.
Note: If performing a tumor biopsy on or after progression on prior line of therapy is not clinically feasible or advisable, the participant may be allowed to enroll upon agreement with the investigator and the Medical Monitor. Biopsies obtained before receiving prior line of therapy may be permitted if a recent biopsy is not feasible and upon agreement with the investigator and the Medical Monitor.
3) Testing for EGFR and ALK is required. Testing for other actionable genomic alterations is recommended and to be performed as per local standard of care and availability of targeted treatment.
Note: Participants with EGFR, ALK, or any other known actionable genomic alterations must have also received treatment with at least 1 approved TKI appropriate to the genomic alteration.
4) Participants must have progressed or experienced disease recurrence after receiving platinum-based chemotherapy in combination with anti-PD-1 or anti-PD-L1 antibody or sequential treatment with platinum-based chemotherapy and anti-PD-1 or anti-PD-L1 antibody (in any order).
Note: Includes participants who received prior platinum-based chemoradiotherapy (with or without maintenance anti-PD-L1 antibody) for Stage III disease. To be considered to have progressed during or after prior treatment with platinum-based chemotherapy, participants should have either received prior platinum-based chemotherapy in the recurrent/metastatic setting or have experienced disease progression within 6 months of last dose of platinum based chemotherapy administered as part of concurrent chemoradiation for Stage III disease or as neoadjuvant or adjuvant therapy. To be considered to have progressed during or after prior treatment with an anti-PD-L1 antibody, participants should have either received this therapy in the recurrent/metastatic setting or have experienced disease progression during “maintenance” treatment following concurrent chemoradiation for Stage III disease or as adjuvant or neoadjuvant treatment for early-stage NSCLC.
5) Documented radiographic disease progression while on or after receiving the most recent treatment regimen for advanced or metastatic NSCLC.
Note: Participants who discontinued most recent treatment due to intolerable toxicity while enrolled on another substudy within this master protocol are permitted.

### Exclusion Criteria

Subjects who meet any of the following exclusion criteria are not eligible to be enrolled in this study:
1) Previously received lung cancer treatment with any of the following:
   a) Topoisomerase 1 inhibitors. Any agent including an ADC containing a chemotherapeutic agent targeting topoisomerase 1.
   b) Trop-2-targeted therapy.
   c) Docetaxel as monotherapy or in combination with other agents.
2) Have an active autoimmune disease that required systemic treatment in the past 2 years defined as treatment with disease-modifying agents, corticosteroids, or immunosuppressive drugs. Replacement therapy (e.g., thyroxine, insulin, or physiologic corticosteroid replacement therapy for adrenal or pituitary insufficiency) is not considered a form of systemic treatment.
3) Has had an allogenic tissue/solid organ transplant.
4) Previously known or existing FLT3 mutation of any kind.
   Rationale for this Study

Sacituzumab govitecan has been evaluated as a monotherapy in patients with metastatic NSCLC in 2 clinical studies: IMMU-132-01 (completed; 54 patients enrolled) and IMMU-132-11 (ongoing; preliminary results are not available yet). In IMMU-132-01, objective response rate (ORR) based on local response assessment was 16.7% for the NSCLC population; all responses were partial responses (PRs). The majority of patients had at least a 30% reduction in the size of the target lesion. Median duration of response (DOR) by local assessment was 6.0 months (range: 2.5 to 21.0). The Kaplan-Meier estimate of the percentage of patients with a response of 6 months was 44.4% (95% CI: 13.6, 71.9).

The therapeutic hypothesis of FLT3L-Fc fusion protein is that FLT3 agonism promotes T-cell infiltration into the tumor in response to dendritic cell (DC) recruitment and expansion of intratumoral DCs. The GS-US-496-5657 study, which is described in Example 32, is designed to assess the safety, tolerability, PK, and preliminary efficacy of FLT3L-Fc fusion protein given as a monotherapy and determine the maximum tolerated dose (MTD). The study also explores the PK and PK-PD relationship as evaluated by peripheral DC expansion. As discussed in Example 32, 9 total patients have enrolled in 3 dose escalation cohorts (2,000 μg, 6,000 μg, and 12,000 μg). To date, no DLTs or discontinuation due to AE have been observed. Three patients had Grade≥3 AEs which were also recorded as SAEs, none of which were considered related to FLT3L-Fc fusion protein. No deaths related to FLT3L-Fc fusion protein have been reported.

The rationale for the sacituzumab govitecan and FLT3L-Fc fusion protein combination treatment is based on their synergistic mechanisms of action. Immunogenic cell death from the sacituzumab govitecan treatment is hypothesized to provide immunostimulation to conventional dendritic cells (type 1) expanded by FLT3L-Fc fusion protein which may, in turn, improve the response to treatment. Taken together, the proposed dosing regimen for sacituzumab govitecan and FLT3L-Fc fusion protein in this study is expected to be safe and efficacious.

### Risk/Benefit Assessment for the Study

Despite recent improvements in the outcomes of patients with advanced or metastatic NSCLC with the development of immune checkpoint inhibitors, there remains a significant unmet medical need as the overall survival of this group of patients remains poor. Even though much progress has been made in development of targeted treatments for specific genomic alterations, eventually most patients have progressive disease on these treatments after which they are treated with platinum chemotherapy and immune checkpoint inhibitors. For patients whose cancers have failed the aforementioned treatments, very few treatment options exist, and they represent a patient population of high unmet medical need. Therefore, there is a need to identify and develop new combination regimens that are more efficacious and less toxic than currently available treatments. However, it cannot be guaranteed that participants in clinical studies will benefit directly from the study treatment or participation.

Given the ongoing need for novel agents and combinations in the treatment of second-line NSCLC, the benefit/risk balance for this study is considered positive.

### Pharmacokinetics and Immunogenicity Assessments

Serum samples are collected for study drug concentration and, if applicable, immunogenicity as outlined in Tables 40 and 41. Additionally, ad hoc samples may be collected at any time if there is a suspected safety issue. The following sacituzumab govitecan-related PK analytes are evaluated at the specified time points: sacituzumab govitecan, total SN-38, free SN-38, and total antibody.

Samples collected for analysis of study drug concentration and immunogenicity may also be used to evaluate safety or efficacy aspects related to concerns arising during or after the study. For participants with positive ADA at the 100-day follow-up visit, additional blood ADA samples may be collected every 4 months (±1 month) up to 1 year from the last dose of study drug, or until the ADA becomes negative, or until the participant starts a subsequent anticancer therapy, or withdraws consent from the study, whichever occurs first.

### UGT1A1 Genotype

UGT1A1 genotype is evaluated from a blood sample collected according to the Tables 40 and 41.

### Biomarker Testing at Enrollment

Local tumor tissue or liquid biopsy testing will be conducted according to Tables 40 and 41 if EGFR or ALK status is unknown. If local testing is unavailable, tumor tissue testing can be performed by the central laboratory. Results must be available prior to enrollment. Diagnostic testing for EGFR and ALK alterations in the central laboratory to determine eligibility at screening will be performed with tests that are approved. EGFR testing will be performed using the Cobas® EGFR Mutation Test (Roche). For ALK, the Vysis ALK Break Apart FISH probe test (Abbott) will be used.

### Biomarker Samples to Address the Study Objectives

Biological specimens are collected from all participants who have provided consent to participate in these studies and may be used to evaluate the association of systemic and/or tissuebased biomarkers with study drug response (including efficacy and/or AEs), resistance, and/or dosage selection, and to better understand the biological pathways, biology of lung cancer and/or the validation of a companion diagnostic for lung cancer or study treatments. Because biomarker science is a rapidly evolving area of investigation, and AEs in particular are difficult to predict, it may not be possible to prospectively specify all tests that may be performed on the specimens provided. The specific analyses include but may not be limited to the biomarkers and assays listed below. The testing outlined below is based upon the current state of scientific knowledge. It may be modified during or after the end of the study to remove tests no longer indicated and/or to add new tests based upon new state-of-the-art knowledge.

Biomarkers (in blood and tissue) may include, but are not limited to, protein expression, analyses of specific immune and tumor signatures (RNA), as well as tumor mutational burden and tumor mutations (DNA). Tumor and blood samples are collected to measure biomarkers of response and resistance and to better understand molecular attributes predictive of treatment in lung cancer. Examples may include, but are not be limited to, PD-L1 and Trop-2-expression, other proteins as well as mutations/gene expression (WES/RNAseq) related to any of the study treatments or related to lung cancer, tumor mutational burden, oncogenic mutations, composition of immune subsets in tumor microenvironment, and pathological features of the tumor. The time points of biomarker sample collection are specified in Tables 40 and 41.

Mandatory blood specimen are collected for the extraction of DNA for genomic testing (genomic sequencing and ctDNA) and correlation with response, as well as control samples for tumor genetic analyses and other exploratory biomarkers. Samples are also used for genotyping to test for polymorphisms of genes that could regulate or be involved in the disposition of sacituzumab govitecan. The control blood sample for genomic sequencing should be collected on Day 1, before administration of the first dose of study drug, but may be collected at any time during the study, if necessary. The sample for ctDNA and plasma should be collected at predose on Cycle 1 Day 1 (C1D1), multiple times on treatment, and at progression/EOT. Details on sample collection are specified in Tables 40 and 41.

A mandatory tumor sample is collected for all participants at screening. If an archival sample already exists that was obtained prior to enrollment, it can be used, if it was obtained after first-line treatment and the participant received no antitumor therapy between sample collection and enrollment. If performing a tumor biopsy prior to enrollment is not clinically feasible or advisable, the participant may be allowed to enroll upon agreement with the investigator and the Gilead Medical Monitor. If no data at the site are available on EGFR and ALK alterations, the samples are also used to test for EGFR and ALK aberrations to determine eligibility. The tumor samples are also used to determine TROP-2 and PD-L1 expression and explore other biomarkers as described herein. Tumor tissue is collected preferentially as tumor blocks, if archival. If not available, freshly sectioned unstained slides may be submitted. If a new biopsy is collected, the whole biopsy is provided. Bone biopsies, fine needle aspirates, and cytology samples are not acceptable samples. Samples from irradiated tumors or samples with limited tumor content are not be acceptable. Please refer to the Laboratory Manual for more detail. In addition to the diagnostic testing for EGFR/ALK aberrations, the tumor samples may be evaluated for other exploratory biomarkers that include, but are not limited to, immunohistochemistry staining, including but not limited to PD-L1 and Trop2, RNA sequencing, and/or whole-exome sequencing. An optional on treatment/EOT/progression biopsy is collected to explore markers of response or resistance to study treatments and changes of biomarkers on treatment.

### Analysis Conventions

### Analysis Sets

This study includes the following analysis sets:

Preliminary Stage Efficacy Set: All participants to be randomized into an experimental treatment arm (i.e., combination arm) in the preliminary stage who take at least 1 dose of each study drug. This is the primary analysis set for efficacy analyses for the preliminary stage. Participants who discontinue without having taken at least 1 dose of study drug are replaced.

Full Analysis Set: Defined for an experimental combination therapy by combining the corresponding PSE Analysis Set and ESITT Analysis Set. This analysis set is used for sensitivity efficacy analyses.

Safety Analysis Set: All participants who receive at least one dose of any study treatment, based on the treatment actually received.

PK Analysis Set is defined for a study treatment, and includes participants to be randomized into the treatment arm in the preliminary and expansion stages and receive at least 1 dose of study drug and have at least 1 measurable post-treatment serum/plasma concentration

Biomarker Analysis Set: All participants who have at least one biomarker data point and receive at least one dose of study treatment Demographic and Baseline Characteristics Analysis

Demographic and baseline measurements are summarized by treatment arm using standard descriptive methods.

### Efficacy Analysis

### Analysis of Primary Efficacy Endpoint

In the preliminary stage, the primary analysis of ORR assessed by the investigator per RECIST version 1.1 is conducted in the PSE Analysis Set. Participants with no postbaseline disease assessment are considered as nonresponders. A 90% CI is calculated for ORR using the exact binomial distribution (Clopper C J, Pearson E S. The Use of Confidence or Fiducial Limits Illustrated in the Case of the Binomial. Biometrika 1934; 26 (4):404-13).

In the expansion stage, the primary analysis of ORR assessed by the investigator per RECIST version 1.1 is conducted in the ESITT Analysis Set; sensitivity analysis is conducted in the FAS. Participants with no postbaseline disease assessment are considered as nonresponders. Based on stratification at randomization at the beginning of expansion stage, strataadjusted difference of ORR between the experimental treatment and the comparator treatment, using the Miettinen-Nurminen method adjusted by the inverse-variance weight, and its 90% CI are calculated (Agresti A. Categorical Data Analysis, 3rd Edition. 3rd Edition ed. Hoboken, N.J.: John John Wiley & Sons Inc.; 2013; Miettinen O, Nurminen M. Comparative analysis of two rates. Stat Med 1985; 4 (2):213-26). A 90% CI is also be calculated for ORR using the exact binomial distribution (Clopper 1934).

### Analysis of Secondary Efficacy Endpoints

In the preliminary stage, analysis of duration of response (DOR) is performed using the Kaplan-Meier method based on participants who achieved objective response in the PSE Analysis Set. Median, Q1 and Q3 are provided, along with the corresponding 90% CI. In the expansion stage, analysis of DOR is performed using the same method in the ESITT Analysis Set and FAS.

For the expansion stage, the analysis of PFS and OS are performed in the ESITT Analysis Set. The treatment effect is estimated by hazard ratio along with its 90% CI using the Cox proportional hazards regression model stratified by the stratification factors at randomization. Kaplan-Meier curves are provided by treatment arm. Median, Q1, Q3 of PFS (and OS, respectively), and the probability of progression-free survival (and overall survival, respectively) at selected landmark points are provided along with the corresponding 90% CIs using the Kaplan-Meier method. A sensitivity analysis of PFS and OS are performed in the FAS.

If the experimental combination therapy does not enter the expansion stage, the primary analysis of PFS and OS is performed using the Kaplan-Meier method in the PSE Analysis Set. Medians, Q1, Q3 of PFS (and OS, respectively), the probability of progression-free survival (and overall survival, respectively) at selected landmark points is provided along with corresponding 90% CIs.

The PFS time is censored at date of the last evaluable assessment prior to subsequent anticancer therapy, if any, or at date of the last evaluable assessment prior to missed or not evaluable (NE) assessments if documented disease progression or death occurs after ≥2 consecutively missed or NE disease assessments. The detailed PFS censoring rule is specified in the SAP.

### Safety Analysis

The treatment-emergent period is defined as the time period from the first dose of study treatment to the earlier of 100 days following the last dose of study treatment or the initiation of subsequent anticancer therapy. Treatment emergent adverse event (TEAE) incidence are summarized in tables. Relevant changes in vital signs and clinical laboratory parameters are analyzed.

The safety variables to be analyzed include exposure to study treatment, AEs, deaths, clinical laboratory test results (hematology and chemistry), physical examination, vital sign measurements, and ECGs. In general, continuous variables are summarized using descriptive statistics (n, mean, median, standard deviation, standard error, and range). Categorical variables are summarized using frequencies and percentages. No formal statistical testing is planned.

Incidence of treatment-emergent laboratory abnormalities, defined as values that increase at least 1 toxicity grade from baseline at any time after baseline up to and including the date of last dose of study treatment [plus 100 days]), are summarized by treatment group. If baseline data are missing, any graded abnormality (i.e., at least a Grade 1) is considered treatment emergent. Laboratory abnormalities that occur before the first dose of study drug or after the participant has been discontinued from treatment for at least 30 days are included in a data listing.

### Pharmacokinetic Analysis

Exploratory evaluations of plasma concentrations and PK parameters (ie, Cmax, Ctrough) of study drugs over time are listed and summarized for all study treatments using descriptive statistics, based on the PK Analysis Set.

Data from this study may be combined with data from other studies with study treatment for population PK and exposure response analyses. If applicable, results from such analyses may be summarized in a separate report, rather than in a clinical study report. Population PK and exposure response analyses of data from this study only may not be conducted.

### Biomarker Analysis

Exploratory biomarker analyses, including change in the biomarkers in response to treatment and correlation of clinical response with biomarkers at baseline and/or on treatment/progression, are based on the appropriate corresponding Biomarker Analysis Set. The data derived from biomarker samples is analyzed depending on the available data, particularly in relationship to clinical outcomes and safety. The result of the biomarker assessment is reported either in the CSR or separately in a scientific report or publication.

### Immunogenicity Analysis

The rate of anti-drug antibody positivity over time is evaluated for each study drug, if applicable, both in each treatment arm and for the combined population based on the Immunogenicity Analysis Set. Detailed summary of ADA prevalence, incidence (treatment-emergent and treatment-boosted), transience, and persistence is calculated by treatment arm and for the combined participant population. Titer summaries at each time point per participant may also be produced for ADA-positive participants. If the ADA is further characterized as neutralizing or otherwise, the overall rate of neutralizing antibody occurrence is also reported.

Exploratory evaluations may be conducted to determine the relationship between immunogenicity assay positivity and one or more safety, PK, or efficacy parameters. These analyses and any others may be reported in a separate PK/ADA report, which are provided as an appendix to the clinical study report. ADA-positive samples may be analyzed further in a validated neutralizing antibody assay, if applicable.

### Description and Handling of FLT3L-Fc Fusion Protein

FLT3L-Fc fusion protein is supplied in two presentations as a liquid or a lyophilized drug product and intended for IV administration.

### Lyophilized Formulation

The FLT3L-Fc fusion protein 10 mg lyophilized powder for reconstitution is a sterile, preservative-free, white to slightly yellow powder composed of FLT3L-Fc fusion protein, histidine buffer, sucrose, and polysorbate 80, with a pH of 5.9 after reconstitution.

The FLT3L-Fc fusion protein lyophilized powder for reconstitution is filled into single-use 20R Type I, clear glass vials, closed with coated elastomeric stoppers, and sealed with aluminum overseals and flip-off caps. Each vial is manufactured to contain 10 mg of lyophilized drug product to be reconstituted with sterile water for injection. The quantity in each vial ensures a minimum total volume of 5.0 mL can be withdrawn after reconstitution per instructions. The reconstituted drug product is intended for IV delivery.

All labels for FLT3L-Fc fusion protein lyophilized powder for reconstitution drug products to be distributed to centers in the United States (US) and other participating countries shall be labeled to meet applicable requirements of the US Food and Drug Administration (FDA), European Union (EU) guideline to Good Manufacturing Practice—Annex 13 (Investigational Medicinal Products), and/or other local regulations.

### Liquid Formulation

FLT3L-Fc fusion protein injection is formulated as a sterile, clear, preservative-free liquid composed of 20 mM histidine/histidine-HCl, 263 mM sucrose, and 0.02% (weight-to-volume ratio [w/v]) polysorbate 80 at pH 5.9. It is supplied in a 6 mL vial with a deliverable volume of 5 mL containing 10 mg of FLT3L-Fc fusion protein at a concentration of 2 mg/mL.

FLT3L-Fc fusion protein should be stored at 2° C. to 8° C. Storage conditions are specified on the study drug label. Until dispensed to the subject, all study drug should be stored in a securely locked area, accessible only to authorized site personnel.

To ensure stability and proper identification, the study drug should be stored in the containers in which they were supplied until dosing the subject.

Consideration should be given to handling, preparation, and disposal through measures that minimize drug contact with the body. Appropriate precautions should be followed to avoid direct eye contact or exposure when handling.

### Description and Handling of Sacituzumab Govitecan

Sacituzumab govitecan is supplied as a sterile, off-white to yellowish lyophilized powder in single-dose glass vials. It is formulated in 2-(N-morpholino) ethane sulfonic acid (MES) buffer containing trehalose and polysorbate 80 and contains no preservatives. Following reconstitution, the concentration of sacituzumab govitecan is 10 mg/mL. The pH of the reconstituted solution is approximately 6.5. Each vial is manufactured to ensure a deliverable quantity of 180 mg of sacituzumab govitecan.

Sacituzumab govitecan is packaged in single-use, 50R, glass vials, closed with coated elastomeric stoppers and capped with flip-off caps with aluminum overseals.

Study drug(s) to be distributed to centers in the US and other participating countries shall be labeled to meet applicable requirements of the US FDA, EU Guidelines to Good Manufacturing Practice, Medicinal Products for Human and Veterinary Use, Annex 13 (Investigational Medicinal Products), and/or other local regulations.

The glass vials of sacituzumab govitecan must be stored under refrigeration (2° C. to 8° C.) and protected from light until use. Since the formulated drug product contains no preservative, vials should be used only once. Refer to the current version of the Pharmacy Manual for additional details.

TABLE 40
Study Protocol Table: Sacituzumab
Govitecan + FLT3L-Fc fusion protein (Combination Arm)

| | | | | | | Safety | Survival |
|---|---|---|---|---|---|---|---|
| Treatment | | | | | | Follow- | Follow- |
| Cycle (Window) | | | | | | up | up |
| | | | C2 and | | | 100 days | Every |
| C1 | | | beyond | | | after | 12 |
| D1 | D8 | D15 | D1 | D8 | EOT | last dose | weeks |
| Windows +/−days | | | | | | | |
| 0 | 3 | 3 | 3 | 3 | 7 | 7 | 14 |

Administrative Procedures

| | |
|---|---|
| Randomization³ | X |

Clinical Procedures/Assessments

| Focused physical | X^{b} | X | X | X |
|---|---|---|---|---|
| examination | | | | |
| Concomitant | Throughout study | | | |
| medications | | | | |
| Vital signs, | X | X | X | X |
| weight | | | | |
| ECOG | X | X | X | X |
| performance | | | | |
| status | | | | |
| 12-lead ECG^{c} | X | X | | |

Imaging/Efficacy Assessments

| | |
|---|---|
| Tumor imaging | Imaging is performed Q6W from |
| and response | C1D1 for the first 48 weeks and |
| assessments | then Q12W until PD as assessed |
| (CT/MRI)^{d} | by the investigator according to |
| | RECIST version 1.1 or initiation of |
| | subsequent anticancer therapy. |
| Bone scan, bone | As clinically indicated |
| MRI, or 18F- | |
| FDG PET scan^{e} | |
| Brain MRI^{d} | As clinically indicated |

Safety Assessments

| | |
|---|---|
| Review of | Collect all AEs and SAEs per |
| adverse events | reporting window in Table 42 |

Laboratory Procedures/Assessments^{f}

| Hematology | X^{g} | X | X | X | X | X | X |
|---|---|---|---|---|---|---|---|
| (Table 43) | | | | | | | |
| Serum chemistry | X | X | X | X | X | X | X |
| including LFTs | | | | | | | |
| (Table 43) | | | | | | | |
| Urinalysis | | | | X | | X | X |
| (Table 43) | | | | | | | |
| TSH, total T3, | X | | | X^{h} | | | |
| free T4 | | | | | | | |
| Urine or serum | X | | | X | | X | X |
| pregnancy testⁱ | | | | | | | |
| Tumor tissue^{j} | X | | | | | X | |
| Genomics whole | X | | | | | | |
| blood biomarker | | | | | | | |
| sample^{k} | | | | | | | |
| Blood biomarker | X | | | X^{l} | | X | |
| (ctDNA | | | | | | | |
| analysis)^{l} | | | | | | | |
| Blood biomarker | X | | | X^{l} | | X | |
| (EDTA plasma)^{l} | | | | | | | |
| Blood sample for | X | | | | | | |
| UGT genotype | | | | | | | |

| | |
|---|---|
| PK samples: | X^{m} |
| sacituzumab | |
| govitecan^{m} | |
| PK samples: | Xⁿ |
| FLT3L-Fc | |
| fusion proteinⁿ | |

| | | |
|---|---|---|
| Immunogenicity: | X^{o} | X^{o} |
| sacituzumab | | |
| govitecan^{o} | | |
| Immunogenicity: | X^{p} | X^{p} |
| FLT3L-Fc | | |
| fusion protein^{p} | | |

Other Assessments

| | |
|---|---|
| Survival follow- | X |
| up | |
| Subsequent | X |
| anticancer | |
| therapy (after | |
| treatment | |
| discontinuation) | |

Study Drug Administration
According to Treatment Group Assignment^{s}

| | | | | |
|---|---|---|---|---|
| Premedication | X | X | X | X |
| for sacituzumab | | | | |
| govitecan for | | | | |
| CINV | | | | |
| Sacituzumab | X | X | X | X |
| govitecan 10 | | | | |
| mg/kg IV^{q} | | | | |
| FLT3L-Fc | X | | X | |
| fusion protein^{r} | | | | |

AE = adverse event;
AUC = area under the curve;
C = Cycle;
CINV = chemotherapy-induced nauseaand vomiting;
CR = complete response;
CT = computed tomography;
ctDNA = circulating tumor DNA;
D = Day;
ECG = electrocardiogram;
ECOG = Eastern CooperativeOncology Group;
EOT = end of treatment;
FDG = F-fluorodeoxyglucose;
FSH = follicle-stimulating hormone;
INR = International normalized ratio;
IV = intravenously;
LFT = liver function test;
MRI = magnetic resonance imaging;
PD = progressive disease;
PET = positron emission tomography;
PR = partial response;
PT = prothrombin time test;
PTT = partial thromboplastin time;
Q3W = every 3 weeks;
QW = weekly;
SAE = serious adverse event;
T3 = triiodothyronine;
T4 = thyroxine;
TSH = thyroid stimulating hormone
^{a}Randomization should occur on CID1 if possible or within 5 days prior.
^{b}The C1D1 physical examination is not required if a complete physical examination was performed within 3 days before study drug administration.
^{c}Abnormal findings should be evaluated as clinically indicated, including repeated ECGs. ECGs may be done at other time points during the study if clinically indicated.
^{d}CT or MRI scans with IV contrast (unless contrast use is medically contraindicated) of chest, abdomen, pelvis, and any other involved disease sites are required in all participants. In participants with stable brain lesion identified as a target lesion for response, brain MRI will also be required at all response assessments. For each participant, the same imaging technique should be used throughout the study. Clinical progression leading to participant discontinuation should be documented by CT or MRI scan if clinically feasible. Participants who discontinue treatment due to toxicity or for any reason other than objective progression will continue to obtain radiologic response assessments according to the protocol-required schedule until PD or initiation of subsequent anticancer therapy. For participants with evidence of CR and PR, a confirmatory scan must be obtained a minimum of 4 weeks after initial documentation of response or at next scheduled assessment. Imaging schedule is based on calendar days. Additional CT or MRI scans may be performed at the discretion of the investigator to assess disease status as medically indicated. These results should be recorded.
^{e}If a participant has known or suspected bone metastasis, a bone scan (99m-technetium polyphosphonate scintigraphy, whole-body bone MRI, or 18F-NaF/FDG PET) to assess bone metastases will be performed within 6 weeks before randomization (historical scans are acceptable). In participants whose body CT/MRI scans indicate that CRhas been achieved, a bone scan or 18F-NaF/FDG PET will be required at confirmation of CRto exclude the presence of new bone metastases or if clinically indicated, and will occur within 1 week, but not more than 2 weeks following a CR as assessed by the investigator. For each participant, the same imaging technique used at screening should be used throughout the study to ensure comparability. Lesions detected on bone scans must be followed with cross-sectional imaging.
^{f}Obtain as clinically indicated. May be obtained more frequently at the discretion of the treating physician if abnormal results warrant follow-up. Results of unscheduled tests should be documented.
^{g}The C1D1 hematology laboratory testing does not need to be conducted if the screening tests were performed within 3 days before study treatment administration.
^{h}TSH, Total T3, and Free T4 is to be collected on Day 1 of odd cycles only.
ⁱThe C1D1 pregnancy test does not need to be repeated if the screening pregnancy test was performed within the 72 hours before study treatment administration. A negative serum pregnancy test must be confirmed for female participants of childbearing potential at screening and prior to dosing on CID 1, and a serum or urine pregnancy test can be performed prior to dosing on Day 1 of each subsequent cycle. Posttreatment pregnancy testing will continue every month after the last dose of study drug up to 6 months after the end of treatment per the duration of required contraception. Testing during the posttreatment period may be done at home and the result self-reported by the participant.
^{j}At C1D1 and at progression/EOT. The on-treatment tumor sample can be obtained at progression or EOT. The progression/EOT tissue sample is optional. Please refer to Laboratory Manual for details.
^{k}Predose at CID1 (can be collected later if missed).
^{l}The biomarker samples for ctDNA and other biomarkers will be collected predose C1D1 and predose at first 3 response assessments (at Weeks 6, 12, and 18 along with the tumor imaging and scans) and at EOT/progression. Please see Laboratory Manual for details.
^{m}Collection times for sacituzumab govitecan PK samples are at predose and after the end of infusion on Day 1 and Day 8 of Cycles 1, 2, 3, 6 and 10. Thereafter, PK samples will be collected at predose on Day 8 every 8 cycles (C18D8, C26D8, etc.) and at the EOT visit. The collection window is −30 minutes prior to the start of infusion of the first drug for predose samples and ±30 minutes at the end of infusion for postdose samples. Ad hoc samples may be collected at any time if there is a suspected safety issue.
ⁿCollection times for FLT3L-Fc fusion protein PK samples are at predose and after the end of infusion on Day 1 and Day 8 (at the same time as sacituzumab govitecan PK collection) on Cycles 1, 2, 3, and 6 and at the EOT visit. The collection window is −30 minutes prior to the start of infusion of the first drug for predose samples and ±30 minutes at the end of infusion for postdose samples. Ad hoc samples may be collected at any time if there is a suspected safety issue.
^{o}Immunogenicity samples for sacituzumab govitecan are collected predose on Day 1 of Cycles 1, 2, 3, 6 and 10. Thereafter, samples will be collected predose on Day 8 every 8 cycles (C18D8, C26D8, etc.) and at the EOT visit. The collection window for the ADA sample is −30 minutes prior to the start of infusion of the first drug for predose samples. If participants permanently discontinue all study treatments, samples will be collected at the 100-day safety follow-up visit. In cases where the safety follow-up visit will not be performed, both PK and immunogenicity samples will be collected at EOT visit. For participants with positive ADA at the 100-day follow-up visit, additional blood ADA samples may be collected every 4 months (±1 month) up to 1 year from the last dose of study drug, or until the ADA becomes negative, or until the participant starts a subsequent anticancer therapy, or withdraws consent from the study, whichever occurs first,
^{p}Immunogenicity samples for FLT3L-Fc fusion protein are collected predose on Day 1 of Cycles 1, 2, 3, 6, and at the EOT visit. The collection window for the ADA sample is −30 minutes prior to the start of infusion of first drug for predose samples. If participants permanently discontinue all study treatments, samples will be collected at the 100-day safety follow-up visit. In cases where the safety follow-up visit will not be performed, both PK and immunogenicity samples will be collected at EOT visit. For participants with positive ADA at the 100-day follow-up visit, additional blood ADA samples may be collected every 4 months (±1 month) up to 1 year from the last dose of study drug, or until the ADA becomes negative, or until the participant starts a subsequent anticancer therapy, or withdraws consent from the study, whichever occurs first.
^{q}Complete blood counts must be obtained prior to each sacituzumab govitecan infusion and treatment should be administered if ANC meets the following criteria: Day 1: ANC ≥1500/mm3, Day 8: ANC ≥1000/mm3. Administer sacituzumab govitecan over 60-120 (±5) minutes followed by a 30-minute observation period followed by FLT3L-Fc fusion protein administered intravenously over 60 minutes. Sacituzumab dosing should continue until PD or unacceptable toxicity.
^{r}Administer FLT3L-Fc fusion protein at a dose of 12,000 ug IV Q3W until Cycle 8.
^{s}Administration of first component of treatment regimen must occur on C1D1. Due to intensive schedule of procedures on C1D1, remainder of study treatment and associated premedications and PK/immunogenicity sampling may flow in to C1D2.

TABLE 41
Study Protocol Table: Sacituzumab Govitecan (Comparator Arm)

| Treatment | | | | | | Safety | Survival |
|---|---|---|---|---|---|---|---|
| Cycle (Window) | | | | | | Follow-up | Follow- |
| | | | C2 and | | | 100 days | up |
| C1 | | | beyond | | | after | Every |
| D1 | D8 | D15 | D1 | D8 | EOT | last dose | 12 weeks |
| Windows +/− days | | | | | | | |
| 0 | 3 | 3 | 3 | 3 | 7 | 7 | 14 |

Administrative Procedures

| | |
|---|---|
| Randomization^{a} | X |

Clinical Procedures/Assessments

| Focused physical | X^{b} | X | X | X |
|---|---|---|---|---|
| examination | | | | |
| Concomitant | Throughout study | | | |
| medications | | | | |
| Vital signs, | X | X | X | X |
| weight | | | | |
| ECOG | X | X | X | X |
| performance | | | | |
| status | | | | |

Imaging/Efficacy Assessments

| | |
|---|---|
| Tumor imaging | Imaging is performed Q6W from |
| and response | C1D1 for the first 48 weeks and then |
| assessments | Q12W until PD as assessed by the |
| (CT/MRI)^{c} | investigator according to RECIST |
| | version 1.1 or initiation of subsequent |
| | anticancer therapy. |
| Bone scan, bone | As clinically indicated |
| MRI, or 18F- | |
| FDG PET scan^{d} | |
| Brain MRI^{c} | As clinically indicated |

Safety Assessments

| | |
|---|---|
| Review of | Collect all AEs and SAEs per |
| adverse events | reporting window in Table 42 |

Laboratory Procedures/Assessmentse

| Hematology | X^{f} | X | X | X | X | X | X |
|---|---|---|---|---|---|---|---|
| (Table 43) | | | | | | | |
| Serum chemistry | X | X | X | X | X | X | X |
| including LFTs | | | | | | | |
| (Table 43) | | | | | | | |
| Urinalysis (Table | | | | X | | X | X |
| 43) | | | | | | | |
| TSH, total T3, | X | | | X^{g} | | | |
| free T4 | | | | | | | |
| Urine or serum | X | | | X | | X | X |
| pregnancy test^{h} | | | | | | | |
| Tumor tissueⁱ | X | | | | | X | |
| Genomics whole | X | | | | | | |
| blood biomarker | | | | | | | |
| sample^{j} | | | | | | | |
| Blood biomarker | X | | | X^{k} | | X | |
| (ctDNA | | | | | | | |
| analysis)^{k} | | | | | | | |
| Blood biomarker | X | | | X^{k} | | X | |
| (EDTA plasma)^{k} | | | | | | | |
| Blood sample for | X | | | | | | |
| UGT genotype | | | | | | | |

| | |
|---|---|
| PK samples^{l} | X^{l} |

| | | |
|---|---|---|
| Immunogenicity^{m} | X^{m} | X^{m} |

Other Assessments

| | |
|---|---|
| Survival | X |
| follow-up | |
| Subsequent | X |
| anticancer | |
| therapy (after | |
| treatment | |
| discontinuation) | |

Study Drug Administration
According to Treatment Group Assignment^{s}

| | | | | |
|---|---|---|---|---|
| Premedication | X | X | X | X |
| for sacituzumab | | | | |
| govitecan for | | | | |
| CINV | | | | |
| Sacituzumab | X | X | X | X |
| govitecan 10 | | | | |
| mg/kg IVⁿ | | | | |

AE = adverse event;
AUC = area under the curve;
C = Cycle;
CINV = chemotherapy-induced nausea and vomiting;
CR = complete response;
CT = computed tomography;
ctDNA = circulating tumor DNA;
D = Day;
ECG = electrocardiogram;
ECOG = Eastern Cooperative Oncology Group;
EOT = end of treatment;
FDG = F-fluorodeoxyglucose;
FSH = follicle-stimulating hormone;
INR = International normalized ratio;
IV = intravenously;
LFT = liver function test;
MRI = magnetic resonance imaging;
PD = progressive disease;
PET = positron emission tomography;
PR = partial response;
PT = prothrombin time test;
PTT = partial thromboplastin time;
Q3W = every 3 weeks;
QW = weekly;
SAE = serious adverse event;
T3 = triiodothyronine;
T4 = thyroxine;
TSH = thyroid stimulating hormone
^{a}Randomization should occur on CID1 if possible or within 5 days prior.
^{b}The C1D1 physical examination is not required if a complete physical examination was performed within 3 days before study drug administration.
^{c}CT or MRI scans with IV contrast (unless contrast use is medically contraindicated) of chest, abdomen, pelvis, and any other involved disease sites are required in all participants. In participants with stable brain lesion identified as a target lesion for response, brain MRI will also be required at all response assessments. For each participant, the same imaging technique should be used throughout the study. Clinical progression leading to participant discontinuation should be documented by CT or MRI scan if clinically feasible. Participants who discontinue treatment due to toxicity or for any reason other than objective progression will continue to obtain radiologic response assessments according to the protocol-required schedule until PD or initiation of subsequent anticancer therapy. For participants with evidence of CR and PR, a confirmatory scan must be obtained a minimum of 4 weeks after initial documentation of response or at next scheduled assessment. Imaging schedule is based on calendar days. Additional CT or MRI scans may be performed at the discretion of the investigator to assess disease status as medically indicated. These results should be recorded.
^{d}If a participant has known or suspected bone metastasis, a bone scan (99m-technetium polyphosphonate scintigraphy, whole-body bone MRI, or 18F-NaF/FDG PET) to assess bone metastases will be performed within 6 weeks before randomization (historical scans are acceptable). In participants whose body CT/MRI scans indicate that CRhas been achieved, a bone scan or 18F- NaF/FDG PET will be required at confirmation of CRto exclude the presence of new bone metastases or if clinically indicated, and will occur within 1 week, but not more than 2 weeks following a CR as assessed by the investigator. For each participant, the same imaging technique used at screening should be used throughout the study to ensure comparability. Lesions detected on bone scans must be followed with cross-sectional imaging.
^{e}Obtain as clinically indicated. May be obtained more frequently at the discretion of the treating physician if abnormal results warrant follow-up. Results of unscheduled tests should be documented,
^{f}The C1D1 hematology laboratory testing does not need to be conducted if the screening tests were
performed within 3 days before study treatment administration.
^{g}TSH, Total T3, and Free T4 is to be collected on Day 1 of odd cycles only.
^{h}The C1D1 pregnancy test does not need to be repeated if the screening pregnancy test was performed within the 72 hours before study treatment administration. A negative serum pregnancy test must be confirmed for female participants of childbearing potential at screening and prior to dosing on CID1, and a serum or urine pregnancy test can be performed prior to dosing on Day 1 of each subsequent cycle. Posttreatment pregnancy testing will continue every month after the last dose of study drug up to 6 months after the end of treatment per the duration of required contraception. Testing during the posttreatment period may be done at home and the result self-reported by the participant.
ⁱAt C1D1 and at progression/EOT. The on-treatment tumor sample can be obtained at progression or EOT. The progression/EOT tissue sample is optional. Please refer to Laboratory Manual for details,
^{j}Predose at CID1 (can be collected later if missed).
^{k}The biomarker samples for ctDNA and other biomarkers will be collected predose C1D1 and predose at first 3 response assessments (at Weeks 6, 12, and 18 along with the tumor imaging and scans) and at EOT/progression. Please see Laboratory Manual for details.
^{l}Collection times for sacituzumab govitecan PK samples are at predose and after the end of infusion on Day 1 and Day 8 of Cycles 1, 2, 3, 6 and 10. Thereafter, PK samples will be collected at predose on Day 8 every 8 cycles (C18D8, C26D8, etc.) and at the EOT visit. The collection window is −30 minutes prior to the start of infusion of the first drug for predose samples and ±30 minutes at the end of infusion for postdose samples. Ad hoc samples may be collected at any time if there is a suspected safety issue.
^{m}Immunogenicity samples for sacituzumab govitecan are collected predose on Day 1 of Cycles 1, 2, 3, 6 and 10. Thereafter, samples will be collected predose on Day 8 every 8 cycles (C18D8, C26D8, etc.) and at the EOT visit. The collection window for the ADA sample is −30 minutes prior to the start of infusion of the first drug for predose samples. If participants permanently discontinue all study treatments, samples will be collected at the 100-day safety follow-up visit. In cases where the safety follow-up visit will not be performed, both PK and immunogenicity samples will be collected at EOT visit. For participants with positive ADA at the 100-day follow-up visit, additional blood ADA samples may be collected every 4 months (±1 month) up to 1 year from the last dose of study drug, or until the ADA becomes negative, or until the participant starts a subsequent anticancer therapy, or withdraws consent from the study, whichever occurs first,
ⁿComplete blood counts must be obtained prior to each sacituzumab govitecan infusion and treatment should be administered if ANC meets the following criteria: Day 1: ANC ≥1500/mm3, Day 8: ANC ≥1000/mm3. Administer sacituzumab govitecan over 60-120 (±5) minutes followed by a 30-minute observation period followed by FLT3L-Fc fusion protein administered intravenously over 60 minutes. Sacituzumab dosing should continue until PD or unacceptable toxicity.
^{o}Administration of first component of treatment regimen must occur on CID1. Due to intensive schedule of procedures on C1D1, remainder of study treatment and associated premedications and PK/immunogenicity sampling may flow in to C1D2.

TABLE 42
Time Period for Collecting AE, SAE, and Other Reportable Safety Event Information

| Reportable Safety Event | Time Period^{a} |
|---|---|
| All SAEs and any AEs related to a | After signing informed consent until first dose of study |
| protocol-mandated procedure | treatment |
| All AEs and SAEs regardless of | Following first dose of study treatment until 100 days |
| causality | after last administration of study treatment |
| Pregnancy in a study participant or | Following initiation of study drug until 6 months after |
| female partner of a study | last administration of study treatment for a female |
| participant | participant or 4 months for female partner of a |
| | participant |
| Special situations reports^{b} | Following first dose of study treatment until 100 days |
| | after last dose of study treatment |

AE = adverse event;
SAE = serious adverse event
^{a}Investigators are not obligated to actively seek AE or SAE information after the end of the reporting period. However, if the investigator learns of any SAE, including a death, at any time after the end of the reporting period, and he/she considers the event to be reasonably related to study treatment or study participation, the event should be promptly reported to the sponsor or its designee. Every effort should be made to follow AEs and SAEs considered related to study treatment or protocol-related procedures until a final outcome can be reported.
^{b}Includes special situations reports that involve Gilead products including concomitant medications that are not considered study drug.

TABLE 43
Laboratory Analytes

| Safety Laboratory Measurements | | | | |
|---|---|---|---|---|
| Chemistry | | | | |
| (Serum or | | Coagu- | | Other Laboratory |
| Plasma) | Hematology | lation | Urinalysis^{a} | Measurements |
| Albumin | Hemoglobin | PT/INR | Specific | TSH, total T3, |
| ALP | Platelet count | PTT or | gravity | and |
| ALT (SGPT) | WBC count | aPTT | pH | free T4 |
| AST (SGOT) | WBC | | Blood | FSH^{c} |
| BUN/urea^{b} | differential | | Glucose | LDH^{d} |
| Calcium | (includes | | Protein | Pregnancy test^{c} |
| Chloride | absolute | | Ketones | Uric acid^{d} |
| Serum | neutrophil | | | Pharmacokinetics |
| Creatinine^{g} | count, | | | ADAs |
| Glucose | absolute | | | Biomarker |
| Magnesium | lymphocyte | | | studies |
| Phosphorus | count, basophil, | | | HIV serology |
| Potassium | eosinophil, | | | Hepatitis B and C |
| Sodium | monocyte) | | | serologies^{f} |
| Total bilirubin | | | | |
| Total protein | | | | |

ADAs = antidrug antibodies;
ALP = alkaline phosphatase;
ALT = alanine aminotransferase;
ANC = absolute neutrophil count;
AST = aspartate aminotransferase;
BUN = blood urea nitrogen;
eGFR = estimated glomerular filtration rate;
FSH = folliclestimulating hormone;
INR = international normalized ratio;
LDH = lactate dehydrogenase;
PT = prothrombin time;
(a)PTT = (activated) partial thromboplastin time;
T3 = triiodothyronine;
T4 = thyroxine;
TSH = thyroid stimulating hormone;
WBC = white blood cell
Refer to Substudy Procedures Tables for collection time points.
^{a}Urinalysis performed on a freshly voided clean sample by dipstick. If dipstick findings are abnormal based on the investigator’s judgment, then a microscopic evaluation will be performed to assess the abnormal findings. Only abnormal results will be captured.
^{b}Sites that perform urea testing instead of BUN testing can capture urea test results.
^{c}Conduct as needed per the substudy protocols for determination of childbearing potential.
^{d}May be tested more frequently at the discretion of the treating physician if abnormal results warrant follow-up. Results of unscheduled tests should be documented.
^{e}In female participants of childbearing potential, pregnancy testing will be performed according to Tables 40 and 41 and as discussed in the substudy protocols.
^{f}HBcAb and HCV antibody if status is unknown; HBsAg, HBV DNA, and/or HCV RNA reflex testing if abnormalities present.
^{g}For creatinine clearance calculation

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXEMPLARY EMBODIMENTS

Exemplary embodiments of the inventions disclosed herein are provided below.

1. A method of preventing, reducing and/or inhibiting the recurrence, growth, proliferation, migration and/or metastasis of a cancer cell or population of cancer cells in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

2. A method of preventing, reducing and/or inhibiting the recurrence, growth, proliferation, migration and/or metastasis of a cancer cell or population of cancer cells in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

3. A method of preventing, reducing and/or inhibiting the recurrence, growth, proliferation, migration and/or metastasis of a cancer cell or population of cancer cells in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

4. A method of preventing, reducing and/or inhibiting the recurrence, growth, proliferation, migration and/or metastasis of a cancer cell or population of cancer cells in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

5. A method of preventing, reducing and/or inhibiting the recurrence, growth, proliferation, migration and/or metastasis of a cancer cell or population of cancer cells in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

6. A method of preventing, reducing and/or inhibiting the recurrence, growth, proliferation, migration and/or metastasis of a cancer cell or population of cancer cells in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

7. A method of preventing, reducing and/or inhibiting the recurrence, growth, proliferation, migration and/or metastasis of a cancer cell or population of cancer cells in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

8. A method of preventing, reducing and/or inhibiting the recurrence, growth, proliferation, migration and/or metastasis of a cancer cell or population of cancer cells in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

9. A method of preventing, reducing and/or inhibiting the recurrence, growth, proliferation, migration and/or metastasis of a cancer cell or population of cancer cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart, and wherein:
a. at least 5 amino acids are truncated from the C-terminus of the FLT3L extracellular domain; and/or
b. the Fc region does not comprise a hinge region.

10. A method of preventing, reducing and/or inhibiting the recurrence, growth, proliferation, migration and/or metastasis of a cancer cell or population of cancer cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart, and wherein:
a. at least 5 amino acids are truncated from the C-terminus of the FLT3L extracellular domain; and/or
b. the Fc region does not comprise a hinge region.

11. A method of preventing, reducing and/or inhibiting the recurrence, growth, proliferation, migration and/or metastasis of a cancer cell or population of cancer cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month, and wherein:
a. at least 5 amino acids are truncated from the C-terminus of the FLT3L extracellular domain; and/or
b. the Fc region does not comprise a hinge region.

12. A method of preventing, reducing and/or inhibiting the recurrence, growth, proliferation, migration and/or metastasis of a cancer cell or population of cancer cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months, and wherein:
a. at least 5 amino acids are truncated from the C-terminus of the FLT3L extracellular domain; and/or
b. the Fc region does not comprise a hinge region.

13. A method of preventing, reducing and/or inhibiting the recurrence, growth, proliferation, migration and/or metastasis of a cancer cell or population of cancer cells in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months, and wherein:
a. at least 5 amino acids are truncated from the C-terminus of the FLT3L extracellular domain; and/or
b. the Fc region does not comprise a hinge region.

14. A method of preventing, reducing and/or inhibiting the recurrence, growth, proliferation, migration and/or metastasis of a cancer cell or population of cancer cells in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

15. A method of preventing, reducing and/or inhibiting the recurrence, growth, proliferation, migration and/or metastasis of a cancer cell or population of cancer cells in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

16. A method of preventing, reducing and/or inhibiting the recurrence, growth, proliferation, migration and/or metastasis of a cancer cell or population of cancer cells in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

17. A method of preventing, reducing and/or inhibiting the recurrence, growth, proliferation, migration and/or metastasis of a cancer cell or population of cancer cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

18. A method of preventing, reducing and/or inhibiting the recurrence, growth, proliferation, migration and/or metastasis of a cancer cell or population of cancer cells in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

19. A method of preventing, reducing and/or inhibiting the recurrence, growth, proliferation, migration and/or metastasis of a cancer cell or population of cancer cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

20. A method of preventing, reducing and/or inhibiting the recurrence, growth, proliferation, migration and/or metastasis of a cancer cell or population of cancer cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

21. A method of treating cancer in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

22. A method of treating cancer in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

23. A method of treating cancer in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

24. A method of treating cancer in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

25. A method of treating cancer in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

26. A method of treating cancer in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

27. A method of treating cancer in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

28. A method of treating cancer in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

29. A method of treating cancer in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart, and wherein:
a. at least 5 amino acids are truncated from the C-terminus of the FLT3L extracellular domain; and/or
b. the Fc region does not comprise a hinge region.

30. A method of treating cancer in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart, and wherein:
a. at least 5 amino acids are truncated from the C-terminus of the FLT3L extracellular domain; and/or
b. the Fc region does not comprise a hinge region.

31. A method of treating cancer in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month, and wherein:
a. at least 5 amino acids are truncated from the C-terminus of the FLT3L extracellular domain; and/or
b. the Fc region does not comprise a hinge region.

32. A method of treating cancer in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months, and wherein:
a. at least 5 amino acids are truncated from the C-terminus of the FLT3L extracellular domain; and/or
b. the Fc region does not comprise a hinge region.

33. A method of treating cancer in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months, and wherein:
a. at least 5 amino acids are truncated from the C-terminus of the FLT3L extracellular domain; and/or
b. the Fc region does not comprise a hinge region.

34. A method of treating cancer in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

35. A method of treating cancer in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

36. A method of treating cancer in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

37. A method of treating cancer in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

38. A method of treating cancer in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

39. A method of treating cancer in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

40. A method of treating cancer in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

41. A method of enhancing, promoting, and/or increasing the tumor infiltration of T-cells and/or NK cells in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

42. A method of enhancing, promoting, and/or increasing the tumor infiltration of T-cells and/or NK cells in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

43. A method of enhancing, promoting, and/or increasing the tumor infiltration of T-cells and/or NK cells in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

44. A method of enhancing, promoting, and/or increasing the tumor infiltration of T-cells and/or NK cells in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

45. A method of enhancing, promoting, and/or increasing the tumor infiltration of T-cells and/or NK cells in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

46. A method of enhancing, promoting, and/or increasing the tumor infiltration of T-cells and/or NK cells in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

47. A method of enhancing, promoting, and/or increasing the tumor infiltration of T-cells and/or NK cells in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

48. A method of enhancing, promoting, and/or increasing the tumor infiltration of T-cells and/or NK cells in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

49. A method of enhancing, promoting, and/or increasing the tumor infiltration of T-cells and/or NK cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart, and

wherein:
a. at least 5 amino acids are truncated from the C-terminus of the FLT3L extracellular domain; and/or
b. the Fc region does not comprise a hinge region.

50. A method of enhancing, promoting, and/or increasing the tumor infiltration of T-cells and/or NK cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart, and

wherein:
a. at least 5 amino acids are truncated from the C-terminus of the FLT3L extracellular domain; and/or
b. the Fc region does not comprise a hinge region.

51. A method of enhancing, promoting, and/or increasing the tumor infiltration of T-cells and/or NK cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month, and

wherein:
a. at least 5 amino acids are truncated from the C-terminus of the FLT3L extracellular domain; and/or
b. the Fc region does not comprise a hinge region.

52. A method of enhancing, promoting, and/or increasing the tumor infiltration of T-cells and/or NK cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months, and

wherein:
a. at least 5 amino acids are truncated from the C-terminus of the FLT3L extracellular domain; and/or
b. the Fc region does not comprise a hinge region.

53. A method of enhancing, promoting, and/or increasing the tumor infiltration of T-cells and/or NK cells in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months, and

wherein:
a. at least 5 amino acids are truncated from the C-terminus of the FLT3L extracellular domain; and/or
b. the Fc region does not comprise a hinge region.

54. A method of enhancing, promoting, and/or increasing the tumor infiltration of T-cells and/or NK cells in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

55. A method of enhancing, promoting, and/or increasing the tumor infiltration of T-cells and/or NK cells in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

56. A method of enhancing, promoting, and/or increasing the tumor infiltration of T-cells and/or NK cells in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

57. A method of enhancing, promoting, and/or increasing the tumor infiltration of T-cells and/or NK cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

58. A method of enhancing, promoting, and/or increasing the tumor infiltration of T-cells and/or NK cells in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

59. A method of enhancing, promoting, and/or increasing the tumor infiltration of T-cells and/or NK cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

60. A method of enhancing, promoting, and/or increasing the tumor infiltration of T-cells and/or NK cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

61. A method of enhancing, promoting, and/or accelerating the recovery from or reversing the effects of lymphopenia in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

62. A method of enhancing, promoting, and/or accelerating the recovery from or reversing the effects of lymphopenia in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

63. A method of enhancing, promoting, and/or accelerating the recovery from or reversing the effects of lymphopenia in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

64. A method of enhancing, promoting, and/or accelerating the recovery from or reversing the effects of lymphopenia in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

65. A method of enhancing, promoting, and/or accelerating the recovery from or reversing the effects of lymphopenia in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

66. A method of enhancing, promoting, and/or accelerating the recovery from or reversing the effects of lymphopenia in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

67. A method of enhancing, promoting, and/or accelerating the recovery from or reversing the effects of lymphopenia in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

68. A method of enhancing, promoting, and/or accelerating the recovery from or reversing the effects of lymphopenia in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

69. A method of enhancing, promoting, and/or accelerating the recovery from or reversing the effects of lymphopenia in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart, and

wherein:
a. at least 5 amino acids are truncated from the C-terminus of the FLT3L extracellular domain; and/or
b. the Fc region does not comprise a hinge region.

70. A method of enhancing, promoting, and/or accelerating the recovery from or reversing the effects of lymphopenia in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart, and

wherein:
a. at least 5 amino acids are truncated from the C-terminus of the FLT3L extracellular domain; and/or
b. the Fc region does not comprise a hinge region.

71. A method of enhancing, promoting, and/or accelerating the recovery from or reversing the effects of lymphopenia in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month, and

wherein:
a. at least 5 amino acids are truncated from the C-terminus of the FLT3L extracellular domain; and/or
b. the Fc region does not comprise a hinge region.

72. A method of enhancing, promoting, and/or accelerating the recovery from or reversing the effects of lymphopenia in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months, and

wherein:
a. at least 5 amino acids are truncated from the C-terminus of the FLT3L extracellular domain; and/or
b. the Fc region does not comprise a hinge region.

73. A method of enhancing, promoting, and/or accelerating the recovery from or reversing the effects of lymphopenia in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months, and

wherein:
a. at least 5 amino acids are truncated from the C-terminus of the FLT3L extracellular domain; and/or
b. the Fc region does not comprise a hinge region.

74. A method of enhancing, promoting, and/or accelerating the recovery from or reversing the effects of lymphopenia in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

75. A method of enhancing, promoting, and/or accelerating the recovery from or reversing the effects of lymphopenia in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

76. A method of enhancing, promoting, and/or accelerating the recovery from or reversing the effects of lymphopenia in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

77. A method of enhancing, promoting, and/or accelerating the recovery from or reversing the effects of lymphopenia in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

78. A method of enhancing, promoting, and/or accelerating the recovery from or reversing the effects of lymphopenia in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

79. A method of enhancing, promoting, and/or accelerating the recovery from or reversing the effects of lymphopenia in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

80. A method of enhancing, promoting, and/or accelerating the recovery from or reversing the effects of lymphopenia in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

81. A method of enhancing, improving, and/or increasing the response to an anticancer therapy in a subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region); and (II) an effective amount of an anticancer agent, wherein:
a. at least 5 amino acids are truncated from the C-terminus of the FLT3L extracellular domain; and/or
b. the Fc region does not comprise a hinge region.

82. A method of enhancing, improving, and/or increasing the response to an anticancer therapy in a subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region); and (II) an effective amount of an anticancer agent, wherein:
a. at least 5 amino acids are truncated from the C-terminus of the FLT3L extracellular domain; and/or
b. the Fc region does not comprise a hinge region.

83. A method of enhancing, improving, and/or increasing the response to an anticancer therapy in a subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region); and (II) an effective amount of an anticancer agent, wherein:
a. at least 5 amino acids are truncated from the C-terminus of the FLT3L extracellular domain; and/or
b. the Fc region does not comprise a hinge region.

84. A method of enhancing, improving, and/or increasing the response to an anticancer therapy in a subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region); and (II) an effective amount of an anticancer agent, wherein:
a. at least 5 amino acids are truncated from the C-terminus of the FLT3L extracellular domain; and/or
b. the Fc region does not comprise a hinge region.

85. A method of enhancing, improving, and/or increasing the response to an anticancer therapy in a human subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region); and (II) an effective amount of an anticancer agent, wherein:
a. at least 5 amino acids are truncated from the C-terminus of the FLT3L extracellular domain; and/or
b. the Fc region does not comprise a hinge region.

86. A method of enhancing, improving, and/or increasing the response to an anticancer therapy in a human subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region); and (II) an effective amount of an anticancer agent, wherein:
a. at least 5 amino acids are truncated from the C-terminus of the FLT3L extracellular domain; and/or
b. the Fc region does not comprise a hinge region.

87. A method of enhancing, improving, and/or increasing the response to an anticancer therapy in a human subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region); and (II) an effective amount of an anticancer agent, wherein:
a. at least 5 amino acids are truncated from the C-terminus of the FLT3L extracellular domain; and/or
b. the Fc region does not comprise a hinge region.

88. A method of enhancing, improving, and/or increasing the response to an anticancer therapy in a human subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region); and (II) an effective amount of an anticancer agent, wherein:
a. at least 5 amino acids are truncated from the C-terminus of the FLT3L extracellular domain; and/or
b. the Fc region does not comprise a hinge region.

89. A method of enhancing, improving, and/or increasing the response to an anticancer therapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart, and (II) an effective amount of an anticancer agent, wherein:
a. at least 5 amino acids are truncated from the C-terminus of the FLT3L extracellular domain; and/or
b. the Fc region does not comprise a hinge region.

90. A method of enhancing, improving, and/or increasing the response to an anticancer therapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart; and (II) an effective amount of an anticancer agent, wherein:
a. at least 5 amino acids are truncated from the C-terminus of the FLT3L extracellular domain; and/or
b. the Fc region does not comprise a hinge region.

91. A method of enhancing, improving, and/or increasing the response to an anticancer therapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month; and (II) an effective amount of an anticancer agent, wherein:
a. at least 5 amino acids are truncated from the C-terminus of the FLT3L extracellular domain; and/or
b. the Fc region does not comprise a hinge region.

92. A method of enhancing, improving, and/or increasing the response to an anticancer therapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months; and (II) an effective amount of an anticancer agent, wherein:
a. at least 5 amino acids are truncated from the C-terminus of the FLT3L extracellular domain; and/or
b. the Fc region does not comprise a hinge region.

93. A method of enhancing, improving, and/or increasing the response to an anticancer therapy in a subject in need thereof, comprising co-administering to the subject (I) between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months; and (II) an effective amount of an anticancer agent,

wherein:
a. at least 5 amino acids are truncated from the C-terminus of the FLT3L extracellular domain; and/or
b. the Fc region does not comprise a hinge region.

94. A method of enhancing, improving, and/or increasing the response to an anticancer therapy in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region); and (II) an effective amount of an anticancer agent, wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

95. A method of enhancing, improving, and/or increasing the response to an anticancer therapy in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region); and (II) an effective amount of an anticancer agent, wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

96. A method of enhancing, improving, and/or increasing the response to an anticancer therapy in a subject in need thereof, comprising co-administering to the subject (I) between about 3 to 8 doses of a fusion protein comprising a human fms related tyrosine kinase 3 ligand (FLT3L) extracellular domain operably linked to an immunoglobulin fragment crystallizable region (Fc region); and (II) an effective amount of an anticancer agent, wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

97. A method of enhancing, improving, and/or increasing the response to an anticancer therapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of an anticancer agent, wherein administering the fusion protein comprises:
A. administering to the subject two or more doses of the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

98. A method of enhancing, improving, and/or increasing the response to an anticancer therapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of an anticancer agent, wherein administering the fusion protein comprises:
A. administering to the subject two or more doses of the fusion protein, wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

99. A method of enhancing, improving, and/or increasing the response to an anticancer therapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of an anticancer agent, wherein administering the fusion protein comprises:
A. administering to the subject two or more doses of an effective amount the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

100. A method of enhancing, improving, and/or increasing the response to an anticancer therapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of an anticancer agent, wherein administering the fusion protein comprises:
A. administering to the subject two or more doses of an effective amount the fusion protein, wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

101. A method of enhancing, improving, and/or increasing the response to sacituzumab govitecan in a subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region); and (II) an effective amount of sacituzumab govitecan, wherein:
a. at least 5 amino acids are truncated from the C-terminus of the FLT3L extracellular domain; and/or
b. the Fc region does not comprise a hinge region.

102. A method of enhancing, improving, and/or increasing the response to sacituzumab govitecan in a subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region); and (II) an effective amount of sacituzumab govitecan, wherein:
a. at least 5 amino acids are truncated from the C-terminus of the FLT3L extracellular domain; and/or
b. the Fc region does not comprise a hinge region.

103. A method of enhancing, improving, and/or increasing the response to sacituzumab govitecan in a subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region); and (II) an effective amount of sacituzumab govitecan, wherein:
a. at least 5 amino acids are truncated from the C-terminus of the FLT3L extracellular domain; and/or
b. the Fc region does not comprise a hinge region.

104. A method of enhancing, improving, and/or increasing the response to sacituzumab govitecan in a subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region); and (II) an effective amount of sacituzumab govitecan, wherein:
a. at least 5 amino acids are truncated from the C-terminus of the FLT3L extracellular domain; and/or
b. the Fc region does not comprise a hinge region.

105. A method of enhancing, improving, and/or increasing the response to sacituzumab govitecan in a human subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region); and (II) an effective amount of sacituzumab govitecan, wherein:
a. at least 5 amino acids are truncated from the C-terminus of the FLT3L extracellular domain; and/or
b. the Fc region does not comprise a hinge region.

106. A method of enhancing, improving, and/or increasing the response to sacituzumab govitecan in a human subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region); and (II) an effective amount of sacituzumab govitecan, wherein:
a. at least 5 amino acids are truncated from the C-terminus of the FLT3L extracellular domain; and/or
b. the Fc region does not comprise a hinge region.

107. A method of enhancing, improving, and/or increasing the response to sacituzumab govitecan in a human subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region); and (II) an effective amount of sacituzumab govitecan, wherein:
a. at least 5 amino acids are truncated from the C-terminus of the FLT3L extracellular domain; and/or
b. the Fc region does not comprise a hinge region.

108. A method of enhancing, improving, and/or increasing the response to sacituzumab govitecan in a human subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region); and (II) an effective amount of sacituzumab govitecan, wherein:
a. at least 5 amino acids are truncated from the C-terminus of the FLT3L extracellular domain; and/or
b. the Fc region does not comprise a hinge region.

109. A method of enhancing, improving, and/or increasing the response to sacituzumab govitecan in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart, and (II) an effective amount of sacituzumab govitecan, wherein:
a. at least 5 amino acids are truncated from the C-terminus of the FLT3L extracellular domain; and/or
b. the Fc region does not comprise a hinge region.

110. A method of enhancing, improving, and/or increasing the response to sacituzumab govitecan in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart; and (II) an effective amount of sacituzumab govitecan, wherein:
a. at least 5 amino acids are truncated from the C-terminus of the FLT3L extracellular domain; and/or
b. the Fc region does not comprise a hinge region.

111. A method of enhancing, improving, and/or increasing the response to sacituzumab govitecan in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month; and (II) an effective amount of sacituzumab govitecan, wherein:
a. at least 5 amino acids are truncated from the C-terminus of the FLT3L extracellular domain; and/or
b. the Fc region does not comprise a hinge region.

112. A method of enhancing, improving, and/or increasing the response to sacituzumab govitecan in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months; and (II) an effective amount of sacituzumab govitecan, wherein:
a. at least 5 amino acids are truncated from the C-terminus of the FLT3L extracellular domain; and/or
b. the Fc region does not comprise a hinge region.

113. A method of enhancing, improving, and/or increasing the response to sacituzumab govitecan in a subject in need thereof, comprising co-administering to the subject (I) between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months; and (II) an effective amount of sacituzumab govitecan, wherein:
a. at least 5 amino acids are truncated from the C-terminus of the FLT3L extracellular domain; and/or
b. the Fc region does not comprise a hinge region.

114. A method of enhancing, improving, and/or increasing the response to sacituzumab govitecan in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region); and (II) an effective amount of sacituzumab govitecan, wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

115. A method of enhancing, improving, and/or increasing the response to sacituzumab govitecan in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region); and (II) an effective amount of sacituzumab govitecan, wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

116. A method of enhancing, improving, and/or increasing the response to sacituzumab govitecan in a subject in need thereof, comprising co-administering to the subject (I) between about 3 to 8 doses of a fusion protein comprising a human fms related tyrosine kinase 3 ligand (FLT3L) extracellular domain operably linked to an immunoglobulin fragment crystallizable region (Fc region); and (II) an effective amount of sacituzumab govitecan, wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

117. A method of enhancing, improving, and/or increasing the response to sacituzumab govitecan in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of sacituzumab govitecan, wherein administering the fusion protein comprises:
A. administering to the subject two or more doses of the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

118. A method of enhancing, improving, and/or increasing the response to sacituzumab govitecan in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of sacituzumab govitecan, wherein administering the fusion protein comprises:
A. administering to the subject two or more doses of the fusion protein, wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

119. A method of enhancing, improving, and/or increasing the response to sacituzumab govitecan in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of sacituzumab govitecan, wherein administering the fusion protein comprises:
A. administering to the subject two or more doses of an effective amount the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

120. A method of enhancing, improving, and/or increasing the response to sacituzumab govitecan in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of sacituzumab govitecan, wherein administering the fusion protein comprises:
A. administering to the subject two or more doses of an effective amount the fusion protein, wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

121. A method of enhancing, improving, and/or increasing the response to magrolimab in a subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region); and (II) an effective amount of magrolimab, wherein:
a. at least 5 amino acids are truncated from the C-terminus of the FLT3L extracellular domain; and/or
b. the Fc region does not comprise a hinge region.

122. A method of enhancing, improving, and/or increasing the response to magrolimab in a subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region); and (II) an effective amount of magrolimab, wherein:
a. at least 5 amino acids are truncated from the C-terminus of the FLT3L extracellular domain; and/or
b. the Fc region does not comprise a hinge region.

123. A method of enhancing, improving, and/or increasing the response to magrolimab in a subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region); and (II) an effective amount of magrolimab, wherein:
a. at least 5 amino acids are truncated from the C-terminus of the FLT3L extracellular domain; and/or
b. the Fc region does not comprise a hinge region.

124. A method of enhancing, improving, and/or increasing the response to magrolimab in a subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region); and (II) an effective amount of magrolimab, wherein:
a. at least 5 amino acids are truncated from the C-terminus of the FLT3L extracellular domain; and/or
b. the Fc region does not comprise a hinge region.

125. A method of enhancing, improving, and/or increasing the response to magrolimab in a human subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region); and (II) an effective amount of magrolimab, wherein:
a. at least 5 amino acids are truncated from the C-terminus of the FLT3L extracellular domain; and/or
b. the Fc region does not comprise a hinge region.

126. A method of enhancing, improving, and/or increasing the response to magrolimab in a human subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region); and (II) an effective amount of magrolimab, wherein:
a. at least 5 amino acids are truncated from the C-terminus of the FLT3L extracellular domain; and/or
b. the Fc region does not comprise a hinge region.

127. A method of enhancing, improving, and/or increasing the response to magrolimab in a human subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region); and (II) an effective amount of magrolimab, wherein:
a. at least 5 amino acids are truncated from the C-terminus of the FLT3L extracellular domain; and/or
b. the Fc region does not comprise a hinge region.

128. A method of enhancing, improving, and/or increasing the response to magrolimab in a human subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region); and (II) an effective amount of magrolimab, wherein:
a. at least 5 amino acids are truncated from the C-terminus of the FLT3L extracellular domain; and/or
b. the Fc region does not comprise a hinge region.

129. A method of enhancing, improving, and/or increasing the response to magrolimab in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart, and (II) an effective amount of magrolimab,

wherein:
a. at least 5 amino acids are truncated from the C-terminus of the FLT3L extracellular domain; and/or
b. the Fc region does not comprise a hinge region.

130. A method of enhancing, improving, and/or increasing the response to magrolimab in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart; and (II) an effective amount of magrolimab, wherein:
a. at least 5 amino acids are truncated from the C-terminus of the FLT3L extracellular domain; and/or
b. the Fc region does not comprise a hinge region.

131. A method of enhancing, improving, and/or increasing the response to magrolimab in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month; and (II) an effective amount of magrolimab,

wherein:
a. at least 5 amino acids are truncated from the C-terminus of the FLT3L extracellular domain; and/or
b. the Fc region does not comprise a hinge region.

132. A method of enhancing, improving, and/or increasing the response to magrolimab in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months; and (II) an effective amount of magrolimab, wherein:
a. at least 5 amino acids are truncated from the C-terminus of the FLT3L extracellular domain; and/or
b. the Fc region does not comprise a hinge region.

133. A method of enhancing, improving, and/or increasing the response to magrolimab in a subject in need thereof, comprising co-administering to the subject (I) between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months; and (II) an effective amount of magrolimab, wherein:
a. at least 5 amino acids are truncated from the C-terminus of the FLT3L extracellular domain; and/or
b. the Fc region does not comprise a hinge region.

134. A method of enhancing, improving, and/or increasing the response to magrolimab in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region); and (II) an effective amount of magrolimab, wherein:
a. each dose comprises between about 200 μg to about 30000 μg of the fusion protein; and

b. at least two of the two or more doses are administered at least 2 weeks apart over a duration of at least 1 month, and

wherein:
a. at least 5 amino acids are truncated from the C-terminus of the FLT3L extracellular domain; and/or
b. the Fc region does not comprise a hinge region.

135. A method of enhancing, improving, and/or increasing the response to magrolimab in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region); and (II) an effective amount of magrolimab, wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

136. A method of enhancing, improving, and/or increasing the response to magrolimab in a subject in need thereof, comprising co-administering to the subject (I) between about 3 to 8 doses of a fusion protein comprising a human fms related tyrosine kinase 3 ligand (FLT3L) extracellular domain operably linked to an immunoglobulin fragment crystallizable region (Fc region); and (II) an effective amount of magrolimab, wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

137. A method of enhancing, improving, and/or increasing the response to magrolimab in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of magrolimab, wherein administering the fusion protein comprises:
A. administering to the subject two or more doses of the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

138. A method of enhancing, improving, and/or increasing the response to magrolimab in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of magrolimab, wherein administering the fusion protein comprises:
A. administering to the subject two or more doses of the fusion protein, wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

139. A method of enhancing, improving, and/or increasing the response to magrolimab in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of magrolimab, wherein administering the fusion protein comprises:
A. administering to the subject two or more doses of an effective amount the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

140. A method of enhancing, improving, and/or increasing the response to magrolimab in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of magrolimab, wherein administering the fusion protein comprises:
A. administering to the subject two or more doses of an effective amount the fusion protein, wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

141. A method of enhancing, improving, and/or increasing the response to an inhibitor of MCL-1 in a subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region); and (II) an effective amount of the MCL-1 inhibitor, wherein:
a. at least 5 amino acids are truncated from the C-terminus of the FLT3L extracellular domain; and/or
b. the Fc region does not comprise a hinge region.

142. A method of enhancing, improving, and/or increasing the response to an inhibitor of MCL-1 in a subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region); and (II) an effective amount of the MCL-1 inhibitor, wherein:
a. at least 5 amino acids are truncated from the C-terminus of the FLT3L extracellular domain; and/or
b. the Fc region does not comprise a hinge region.

143. A method of enhancing, improving, and/or increasing the response to an inhibitor of MCL-1 in a subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region); and (II) an effective amount of the MCL-1 inhibitor, wherein:
a. at least 5 amino acids are truncated from the C-terminus of the FLT3L extracellular domain; and/or
b. the Fc region does not comprise a hinge region.

144. A method of enhancing, improving, and/or increasing the response to an inhibitor of MCL-1 in a subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region); and (II) an effective amount of the MCL-1 inhibitor, wherein:
a. at least 5 amino acids are truncated from the C-terminus of the FLT3L extracellular domain; and/or
b. the Fc region does not comprise a hinge region.

145. A method of enhancing, improving, and/or increasing the response to an inhibitor of MCL-1 in a human subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region); and (II) an effective amount of the MCL-1 inhibitor, wherein:
a. at least 5 amino acids are truncated from the C-terminus of the FLT3L extracellular domain; and/or
b. the Fc region does not comprise a hinge region.

146. A method of enhancing, improving, and/or increasing the response to an inhibitor of MCL-1 in a human subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region); and (II) an effective amount of the MCL-1 inhibitor, wherein:
a. at least 5 amino acids are truncated from the C-terminus of the FLT3L extracellular domain; and/or
b. the Fc region does not comprise a hinge region.

147. A method of enhancing, improving, and/or increasing the response to an inhibitor of MCL-1 in a human subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region); and (II) an effective amount of the MCL-1 inhibitor, wherein:
a. at least 5 amino acids are truncated from the C-terminus of the FLT3L extracellular domain; and/or
b. the Fc region does not comprise a hinge region.

148. A method of enhancing, improving, and/or increasing the response to an inhibitor of MCL-1 in a human subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region); and (II) an effective amount of the MCL-1 inhibitor, wherein:
a. at least 5 amino acids are truncated from the C-terminus of the FLT3L extracellular domain; and/or
b. the Fc region does not comprise a hinge region.

149. A method of enhancing, improving, and/or increasing the response to an inhibitor of MCL-1 in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart, and (II) an effective amount of the MCL-1 inhibitor, wherein:
a. at least 5 amino acids are truncated from the C-terminus of the FLT3L extracellular domain; and/or
b. the Fc region does not comprise a hinge region.

150. A method of enhancing, improving, and/or increasing the response to an inhibitor of MCL-1 in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart; and (II) an effective amount of the MCL-1 inhibitor, wherein:
a. at least 5 amino acids are truncated from the C-terminus of the FLT3L extracellular domain; and/or
b. the Fc region does not comprise a hinge region.

151. A method of enhancing, improving, and/or increasing the response to an inhibitor of MCL-1 in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month; and (II) an effective amount of the MCL-1 inhibitor,

wherein:
a. at least 5 amino acids are truncated from the C-terminus of the FLT3L extracellular domain; and/or
b. the Fc region does not comprise a hinge region.

152. A method of enhancing, improving, and/or increasing the response to an inhibitor of MCL-1 in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months; and (II) an effective amount of the MCL-1 inhibitor, wherein:
a. at least 5 amino acids are truncated from the C-terminus of the FLT3L extracellular domain; and/or
b. the Fc region does not comprise a hinge region.

153. A method of enhancing, improving, and/or increasing the response to an inhibitor of MCL-1 in a subject in need thereof, comprising co-administering to the subject (I) between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months; and (II) an effective amount of the MCL-1 inhibitor, wherein:
a. at least 5 amino acids are truncated from the C-terminus of the FLT3L extracellular domain; and/or
b. the Fc region does not comprise a hinge region.

154. A method of enhancing, improving, and/or increasing the response to an inhibitor of MCL-1 in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region); and (II) an effective amount of the MCL-1 inhibitor, wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

155. A method of enhancing, improving, and/or increasing the response to an inhibitor of MCL-1 in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region); and (II) an effective amount of the MCL-1 inhibitor, wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

156. A method of enhancing, improving, and/or increasing the response to an inhibitor of MCL-1 in a subject in need thereof, comprising co-administering to the subject (I) between about 3 to 8 doses of a fusion protein comprising a human fms related tyrosine kinase 3 ligand (FLT3L) extracellular domain operably linked to an immunoglobulin fragment crystallizable region (Fc region); and (II) an effective amount of the MCL-1 inhibitor, wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

157. A method of enhancing, improving, and/or increasing the response to an inhibitor of MCL-1 in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of the MCL-1 inhibitor, wherein administering the fusion protein comprises:
A. administering to the subject two or more doses of the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

158. A method of enhancing, improving, and/or increasing the response to an inhibitor of MCL-1 in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of the MCL-1 inhibitor, wherein administering the fusion protein comprises:
A. administering to the subject two or more doses of the fusion protein, wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

159. A method of enhancing, improving, and/or increasing the response to an inhibitor of MCL-1 in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of the MCL-1 inhibitor, wherein administering the fusion protein comprises:
A. administering to the subject two or more doses of an effective amount the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

160. A method of enhancing, improving, and/or increasing the response to an inhibitor of MCL-1 in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of the MCL-1 inhibitor, wherein administering the fusion protein comprises:
A. administering to the subject two or more doses of an effective amount the fusion protein, wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

161. A method of promoting, inducing and/or increasing the expansion and/or proliferation of a cell or a population of cells that express fms related tyrosine kinase 3 (FLT3, CD135) in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the expansion and/or proliferation of the cell or population of cells is observed within 5 to 15 days after administration of the fusion protein, and wherein:
a. at least 5 amino acids are truncated from the C-terminus of the FLT3L extracellular domain; and/or
b. the Fc region does not comprise a hinge region.

162. A method of promoting, inducing and/or increasing the expansion and/or proliferation of a cell or a population of cells that express fms related tyrosine kinase 3 (FLT3, CD135) in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the expansion and/or proliferation of the cell or population of cells is observed within 5 to 15 days after administration of the fusion protein, wherein:
a. at least 5 amino acids are truncated from the C-terminus of the FLT3L extracellular domain; and/or
b. the Fc region does not comprise a hinge region.

163. A method of promoting, inducing and/or increasing the expansion and/or proliferation of a cell or a population of cells that express fms related tyrosine kinase 3 (FLT3, CD135) in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the expansion and/or proliferation of the cell or population of cells is observed within 5 to 15 days after administration of the fusion protein, wherein:
a. at least 5 amino acids are truncated from the C-terminus of the FLT3L extracellular domain; and/or
b. the Fc region does not comprise a hinge region.

164. A method of promoting, inducing and/or increasing the expansion and/or proliferation of a cell or a population of cells that express fms related tyrosine kinase 3 (FLT3, CD135) in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the expansion and/or proliferation of the cell or population of cells is observed within 5 to 15 days after administration of the fusion protein, wherein:
a. at least 5 amino acids are truncated from the C-terminus of the FLT3L extracellular domain; and/or
b. the Fc region does not comprise a hinge region.

165. A method of promoting, inducing and/or increasing the expansion and/or proliferation of a cell or a population of cells that express fms related tyrosine kinase 3 (FLT3, CD135) in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the expansion and/or proliferation of the cell or population of cells is observed within 5 to 15 days after administration of the fusion protein, and wherein:
a. at least 5 amino acids are truncated from the C-terminus of the FLT3L extracellular domain; and/or
b. the Fc region does not comprise a hinge region.

166. A method of promoting, inducing and/or increasing the expansion and/or proliferation of a cell or a population of cells that express fms related tyrosine kinase 3 (FLT3, CD135) in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the expansion and/or proliferation of the cell or population of cells is observed within 5 to 15 days after administration of the fusion protein, wherein:
a. at least 5 amino acids are truncated from the C-terminus of the FLT3L extracellular domain; and/or
b. the Fc region does not comprise a hinge region.

167. A method of promoting, inducing and/or increasing the expansion and/or proliferation of a cell or a population of cells that express fms related tyrosine kinase 3 (FLT3, CD135) in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the expansion and/or proliferation of the cell or population of cells is observed within 5 to 15 days after administration of the fusion protein, wherein:
a. at least 5 amino acids are truncated from the C-terminus of the FLT3L extracellular domain; and/or
b. the Fc region does not comprise a hinge region.

168. A method of promoting, inducing and/or increasing the expansion and/or proliferation of a cell or a population of cells that express fms related tyrosine kinase 3 (FLT3, CD135) in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the expansion and/or proliferation of the cell or population of cells is observed within 5 to 15 days after administration of the fusion protein, wherein:
a. at least 5 amino acids are truncated from the C-terminus of the FLT3L extracellular domain; and/or
b. the Fc region does not comprise a hinge region.

169. A method of promoting, inducing and/or increasing the expansion and/or proliferation of a cell or a population of cells that express fms related tyrosine kinase 3 (FLT3, CD135) in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein:
a. at least two of the two or more doses are administered at least two weeks apart; and
b. the expansion and/or proliferation of the cell or population of cells is observed within 5 to 15 days after administration of the fusion protein, and
   wherein:
a. at least 5 amino acids are truncated from the C-terminus of the FLT3L extracellular domain; and/or
b. the Fc region does not comprise a hinge region.

170. A method of promoting, inducing and/or increasing the expansion and/or proliferation of a cell or a population of cells that express fms related tyrosine kinase 3 (FLT3, CD135) in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein:
a. at least two of the two or more doses are administered between 2 to 5 weeks apart; and
b. the expansion and/or proliferation of the cell or population of cells is observed within 5 to 15 days after administration of the fusion protein, and
   wherein:
a. at least 5 amino acids are truncated from the C-terminus of the FLT3L extracellular domain; and/or
b. the Fc region does not comprise a hinge region.
   171. A method of promoting, inducing and/or increasing the expansion and/or proliferation of a cell or a population of cells that express fms related tyrosine kinase 3 (FLT3, CD135) in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein:
a. at least two of the two or more doses are administered at least about 2 weeks apart over a duration of at least about 1 month; and
b. the expansion and/or proliferation of the cell or population of cells is observed within 5 to 15 days after administration of the fusion protein, and
   wherein:
a. at least 5 amino acids are truncated from the C-terminus of the FLT3L extracellular domain; and/or
b. the Fc region does not comprise a hinge region.

172. A method of promoting, inducing and/or increasing the expansion and/or proliferation of a cell or a population of cells that express fms related tyrosine kinase 3 (FLT3, CD135) in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein:
a. at least two of the two or more doses are administered between about 2 to 5 weeks apart over a duration of between about 1 to 4 months; and
b. the expansion and/or proliferation of the cell or population of cells is observed within 5 to 15 days after administration of the fusion protein, and
   wherein:
a. at least 5 amino acids are truncated from the C-terminus of the FLT3L extracellular domain; and/or
b. the Fc region does not comprise a hinge region.

173. A method of promoting, inducing and/or increasing the expansion and/or proliferation of a cell or a population of cells that express fms related tyrosine kinase 3 (FLT3, CD135) in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein:
a. at least two of the doses are administered between about 2 to 5 weeks apart over a duration of between about 1 to 4 months; and
b. the expansion and/or proliferation of the cell or population of cells is observed within 5 to 15 days after administration of the fusion protein, and
   wherein:
a. at least 5 amino acids are truncated from the C-terminus of the FLT3L extracellular domain; and/or
b. the Fc region does not comprise a hinge region.

174. A method of promoting, inducing and/or increasing the expansion and/or proliferation of a cell or a population of cells that express fms related tyrosine kinase 3 (FLT3, CD135) in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein;
b. at least two of the two or more doses are administered at least 2 weeks apart over a duration of at least 1 month; and
c. the expansion and/or proliferation of the cell or population of cells is observed within 5 to 15 days after administration of the fusion protein, and
   wherein:
a. at least 5 amino acids are truncated from the C-terminus of the FLT3L extracellular domain; and/or
b. the Fc region does not comprise a hinge region.

175. A method of promoting, inducing and/or increasing the expansion and/or proliferation of a cell or a population of cells that express fms related tyrosine kinase 3 (FLT3, CD135) in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein;
b. at least two of the two or more doses are administered between about 2 to 5 weeks apart over a duration of between about 1 to 4 months; and
c. the expansion and/or proliferation of the cell or population of cells is observed within 5 to 15 days after administration of the fusion protein, and
   wherein:
a. at least 5 amino acids are truncated from the C-terminus of the FLT3L extracellular domain; and/or
b. the Fc region does not comprise a hinge region.

176. A method of promoting, inducing and/or increasing the expansion and/or proliferation of a cell or a population of cells that express fms related tyrosine kinase 3 (FLT3, CD135) in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein;
b. at least two of the doses are administered between about 2 to 5 weeks apart over a duration of between about 1 to 4 months; and
c. the expansion and/or proliferation of the cell or population of cells is observed within 5 to 15 days after administration of the fusion protein, and
   wherein:
a. at least 5 amino acids are truncated from the C-terminus of the FLT3L extracellular domain; and/or
b. the Fc region does not comprise a hinge region.

177. A method of promoting, inducing and/or increasing the expansion and/or proliferation of a cell or a population of cells that express fms related tyrosine kinase 3 (FLT3, CD135) in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein:
   i. the dosing interval for the two or more doses is one dose every 2 to 4 weeks; and
   ii. the expansion and/or proliferation of the cell or population of cells is observed within 5 to 15 days after administration of the fusion protein, and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

178. A method of promoting, inducing and/or increasing the expansion and/or proliferation of a cell or a population of cells that express fms related tyrosine kinase 3 (FLT3, CD135) in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein:
   i. each dose comprises between about 200 μg to about 30000 μg of the fusion protein;
   ii. the dosing interval for the two or more doses is once every 2 to 4 weeks; and
   iii. the expansion and/or proliferation of the cell or population of cells is observed within 5 to 15 days after administration of the fusion protein, and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

179. A method of promoting, inducing and/or increasing the expansion and/or proliferation of a cell or a population of cells that express fms related tyrosine kinase 3 (FLT3, CD135) in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein:
   i. the dosing interval for the two or more doses is one dose every 2 to 4 weeks; and
   ii. the expansion and/or proliferation of the cell or population of cells is observed within 5 to 15 days after administration of the fusion protein; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

180. A method of promoting, inducing and/or increasing the expansion and/or proliferation of a cell or a population of cells that express fms related tyrosine kinase 3 (FLT3, CD135) in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein:
   i. the two or more doses are administered about 8 to 20 days apart; and
   ii. the expansion and/or proliferation of the cell or population of cells is observed within 5 to 15 days after administration of the fusion protein,
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

181. A method of enhancing, improving, and/or increasing the response to an immunotherapy in a subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region); and (II) an effective amount of the immunotherapy, wherein:
a. at least 5 amino acids are truncated from the C-terminus of the FLT3L extracellular domain; and/or
b. the Fc region does not comprise a hinge region.

182. A method of enhancing, improving, and/or increasing the response to an immunotherapy in a subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region); and (II) an effective amount of the immunotherapy, wherein:
a. at least 5 amino acids are truncated from the C-terminus of the FLT3L extracellular domain; and/or
b. the Fc region does not comprise a hinge region.

183. A method of enhancing, improving, and/or increasing the response to an immunotherapy in a subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region); and (II) an effective amount of the immunotherapy, wherein:
a. at least 5 amino acids are truncated from the C-terminus of the FLT3L extracellular domain; and/or
b. the Fc region does not comprise a hinge region.

184. A method of enhancing, improving, and/or increasing the response to an immunotherapy in a subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region); and (II) an effective amount of the immunotherapy, wherein:
a. at least 5 amino acids are truncated from the C-terminus of the FLT3L extracellular domain; and/or
b. the Fc region does not comprise a hinge region.

185. A method of enhancing, improving, and/or increasing the response to an immunotherapy in a human subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region); and (II) an effective amount of the immunotherapy, wherein:
a. at least 5 amino acids are truncated from the C-terminus of the FLT3L extracellular domain; and/or
b. the Fc region does not comprise a hinge region.

186. A method of enhancing, improving, and/or increasing the response to an immunotherapy in a human subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region); and (II) an effective amount of the immunotherapy, wherein:
a. at least 5 amino acids are truncated from the C-terminus of the FLT3L extracellular domain; and/or
b. the Fc region does not comprise a hinge region.

187. A method of enhancing, improving, and/or increasing the response to an immunotherapy in a human subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region); and (II) an effective amount of the immunotherapy, wherein:
a. at least 5 amino acids are truncated from the C-terminus of the FLT3L extracellular domain; and/or
b. the Fc region does not comprise a hinge region.

188. A method of enhancing, improving, and/or increasing the response to an immunotherapy in a human subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region); and (II) an effective amount of the immunotherapy, wherein:
a. at least 5 amino acids are truncated from the C-terminus of the FLT3L extracellular domain; and/or
b. the Fc region does not comprise a hinge region.

189. A method of enhancing, improving, and/or increasing the response to an immunotherapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart, and (II) an effective amount of the immunotherapy, wherein:
a. at least 5 amino acids are truncated from the C-terminus of the FLT3L extracellular domain; and/or
b. the Fc region does not comprise a hinge region.

190. A method of enhancing, improving, and/or increasing the response to an immunotherapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart; and (II) an effective amount of the immunotherapy, wherein:
a. at least 5 amino acids are truncated from the C-terminus of the FLT3L extracellular domain; and/or
b. the Fc region does not comprise a hinge region.

191. A method of enhancing, improving, and/or increasing the response to an immunotherapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month; and (II) an effective amount of the immunotherapy, wherein:
a. at least 5 amino acids are truncated from the C-terminus of the FLT3L extracellular domain; and/or
b. the Fc region does not comprise a hinge region.

192. A method of enhancing, improving, and/or increasing the response to an immunotherapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months; and (II) an effective amount of the immunotherapy, wherein:
a. at least 5 amino acids are truncated from the C-terminus of the FLT3L extracellular domain; and/or
b. the Fc region does not comprise a hinge region.

193. A method of enhancing, improving, and/or increasing the response to an immunotherapy in a subject in need thereof, comprising co-administering to the subject (I) between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months; and (II) an effective amount of the immunotherapy, wherein:
a. at least 5 amino acids are truncated from the C-terminus of the FLT3L extracellular domain; and/or
b. the Fc region does not comprise a hinge region.

194. A method of enhancing, improving, and/or increasing the response to an immunotherapy in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region); and (II) an effective amount of the immunotherapy, wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

195. A method of enhancing, improving, and/or increasing the response to an immunotherapy in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region); and (II) an effective amount of the immunotherapy, wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

196. A method of enhancing, improving, and/or increasing the response to an immunotherapy in a subject in need thereof, comprising co-administering to the subject (I) between about 3 to 8 doses of a fusion protein comprising a human fms related tyrosine kinase 3 ligand (FLT3L) extracellular domain operably linked to an immunoglobulin fragment crystallizable region (Fc region); and (II) an effective amount of the immunotherapy, wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

197. A method of enhancing, improving, and/or increasing the response to an immunotherapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of the immunotherapy, wherein administering the fusion protein comprises:
A. administering to the subject two or more doses of the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

198. A method of enhancing, improving, and/or increasing the response to an immunotherapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of the immunotherapy, wherein administering the fusion protein comprises:
A. administering to the subject two or more doses of the fusion protein, wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

199. A method of enhancing, improving, and/or increasing the response to an immunotherapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of the immunotherapy, wherein administering the fusion protein comprises:
A. administering to the subject two or more doses of an effective amount the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

200. A method of enhancing, improving, and/or increasing the response to an immunotherapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region); and (II) an effective amount of the immunotherapy, wherein administering the fusion protein comprises:
A. administering to the subject two or more doses of an effective amount the fusion protein, wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

201. A method of inducing the immune system in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), and wherein:
a. at least 5 amino acids are truncated from the C-terminus of the FLT3L extracellular domain; and/or
b. the Fc region does not comprise a hinge region.

202. A method of inducing the immune system in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

203. A method of inducing the immune system in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

204. A method of inducing the immune system in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

205. A method of inducing the immune system in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), and wherein:
a. at least 5 amino acids are truncated from the C-terminus of the FLT3L extracellular domain; and/or
b. the Fc region does not comprise a hinge region.

206. A method of inducing the immune system in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

207. A method of inducing the immune system in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein:
a. at least 5 amino acids are truncated from the C-terminus of the FLT3L extracellular domain; and/or
b. the Fc region does not comprise a hinge region.

208. A method of inducing the immune system in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region wherein:
a. at least 5 amino acids are truncated from the C-terminus of the FLT3L extracellular domain; and/or
b. the Fc region does not comprise a hinge region.

209. A method of inducing the immune system in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least two weeks apart; wherein:
a. at least 5 amino acids are truncated from the C-terminus of the FLT3L extracellular domain; and/or
b. the Fc region does not comprise a hinge region.

210. A method of inducing the immune system in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between 2 to 5 weeks apart; and

wherein:
a. at least 5 amino acids are truncated from the C-terminus of the FLT3L extracellular domain; and/or
b. the Fc region does not comprise a hinge region.

211. A method of inducing the immune system in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered at least about 2 weeks apart over a duration of at least about 1 month, and

wherein:
a. at least 5 amino acids are truncated from the C-terminus of the FLT3L extracellular domain; and/or
b. the Fc region does not comprise a hinge region.

212. A method of inducing the immune system in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein at least two of the two or more doses are administered between about 2 to 5 weeks apart over a duration of between about 1 to 4 months, and

wherein:
a. at least 5 amino acids are truncated from the C-terminus of the FLT3L extracellular domain; and/or
b. the Fc region does not comprise a hinge region.

213. A method of inducing the immune system in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein at least two of the doses are administered between about 2 to 5 weeks apart over a duration of between about 1 to 4 months, and

wherein:
a. at least 5 amino acids are truncated from the C-terminus of the FLT3L extracellular domain; and/or
b. the Fc region does not comprise a hinge region.

214. A method of inducing the immune system in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

215. A method of inducing the immune system in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

216. A method of inducing the immune system in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

217. A method of inducing the immune system in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

218. A method of inducing the immune system in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

219. A method of inducing the immune system in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months, and
   wherein:
a. at least 5 amino acids are truncated from the C-terminus of the FLT3L extracellular domain; and/or
b. the Fc region does not comprise a hinge region.

220. A method of inducing the immune system in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

221. A method of preventing, reducing and/or inhibiting the recurrence, growth, proliferation, migration and/or metastasis of a cancer cell or population of cancer cells in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

222. A method of preventing, reducing and/or inhibiting the recurrence, growth, proliferation, migration and/or metastasis of a cancer cell or population of cancer cells in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

223. A method of preventing, reducing and/or inhibiting the recurrence, growth, proliferation, migration and/or metastasis of a cancer cell or population of cancer cells in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

224. A method of preventing, reducing and/or inhibiting the recurrence, growth, proliferation, migration and/or metastasis of a cancer cell or population of cancer cells in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

225. A method of preventing, reducing and/or inhibiting the recurrence, growth, proliferation, migration and/or metastasis of a cancer cell or population of cancer cells in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

226. A method of preventing, reducing and/or inhibiting the recurrence, growth, proliferation, migration and/or metastasis of a cancer cell or population of cancer cells in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L
b. the Fc region does not comprise a hinge region.

227. A method of preventing, reducing and/or inhibiting the recurrence, growth, proliferation, migration and/or metastasis of a cancer cell or population of cancer cells in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

228. A method of preventing, reducing and/or inhibiting the recurrence, growth, proliferation, migration and/or metastasis of a cancer cell or population of cancer cells in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

229. A method of preventing, reducing and/or inhibiting the recurrence, growth, proliferation, migration and/or metastasis of a cancer cell or population of cancer cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the two or more doses are administered at least two weeks apart, and

wherein:
a. at least 5 amino acids are truncated from the C-terminus of the FLT3L extracellular domain; and/or
b. the Fc region does not comprise a hinge region.

230. A method of preventing, reducing and/or inhibiting the recurrence, growth, proliferation, migration and/or metastasis of a cancer cell or population of cancer cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the two or more doses are administered between 2 to 5 weeks apart, and

wherein:
a. at least 5 amino acids are truncated from the C-terminus of the FLT3L extracellular domain; and/or
b. the Fc region does not comprise a hinge region.

231. A method of preventing, reducing and/or inhibiting the recurrence, growth, proliferation, migration and/or metastasis of a cancer cell or population of cancer cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the two or more doses are administered at least about 2 weeks apart over a duration of at least about 1 month, and

wherein:
a. at least 5 amino acids are truncated from the C-terminus of the FLT3L extracellular domain; and/or
b. the Fc region does not comprise a hinge region.

232. A method of preventing, reducing and/or inhibiting the recurrence, growth, proliferation, migration and/or metastasis of a cancer cell or population of cancer cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the two or more doses are administered between about 2 to 5 weeks apart over a duration of between about 1 to 4 months, and

wherein:
a. at least 5 amino acids are truncated from the C-terminus of the FLT3L extracellular domain; and/or
b. the Fc region does not comprise a hinge region.

233. A method of preventing, reducing and/or inhibiting the recurrence, growth, proliferation, migration and/or metastasis of a cancer cell or population of cancer cells in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the doses are administered between about 2 to 5 weeks apart over a duration of between about 1 to 4 months, and

wherein:
a. at least 5 amino acids are truncated from the C-terminus of the FLT3L extracellular domain; and/or
b. the Fc region does not comprise a hinge region.

234. A method of preventing, reducing and/or inhibiting the recurrence, growth, proliferation, migration and/or metastasis of a cancer cell or population of cancer cells in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

235. A method of preventing, reducing and/or inhibiting the recurrence, growth, proliferation, migration and/or metastasis of a cancer cell or population of cancer cells in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

236. A method of preventing, reducing and/or inhibiting the recurrence, growth, proliferation, migration and/or metastasis of a cancer cell or population of cancer cells in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

237. A method of preventing, reducing and/or inhibiting the recurrence, growth, proliferation, migration and/or metastasis of a cancer cell or population of cancer cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

238. A method of preventing, reducing and/or inhibiting the recurrence, growth, proliferation, migration and/or metastasis of a cancer cell or population of cancer cells in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

239. A method of preventing, reducing and/or inhibiting the recurrence, growth, proliferation, migration and/or metastasis of a cancer cell or population of cancer cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

240. A method of preventing, reducing and/or inhibiting the recurrence, growth, proliferation, migration and/or metastasis of a cancer cell or population of cancer cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

241. A method of treating cancer in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

242. A method of treating cancer in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

243. A method of treating cancer in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

244. A method of treating cancer in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

245. A method of treating cancer in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

246. A method of treating cancer in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

247. A method of treating cancer in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

248. A method of treating cancer in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

249. A method of treating cancer in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the two or more doses are administered at least two weeks apart, and

wherein:
a. at least 5 amino acids are truncated from the C-terminus of the FLT3L extracellular domain; and/or
b. the Fc region does not comprise a hinge region.

250. A method of treating cancer in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the two or more doses are administered between 2 to 5 weeks apart, and

wherein:
a. at least 5 amino acids are truncated from the C-terminus of the FLT3L extracellular domain; and/or
b. the Fc region does not comprise a hinge region.

251. A method of treating cancer in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the two or more doses are administered at least about 2 weeks apart over a duration of at least about 1 month, and

wherein:
a. at least 5 amino acids are truncated from the C-terminus of the FLT3L extracellular domain; and/or
b. the Fc region does not comprise a hinge region.

252. A method of treating cancer in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the two or more doses are administered between about 2 to 5 weeks apart over a duration of between about 1 to 4 months, and

wherein:
a. at least 5 amino acids are truncated from the C-terminus of the FLT3L extracellular domain; and/or
b. the Fc region does not comprise a hinge region.

253. A method of treating cancer in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the doses are administered between about 2 to 5 weeks apart over a duration of between about 1 to 4 months, and

wherein:
a. at least 5 amino acids are truncated from the C-terminus of the FLT3L extracellular domain; and/or
b. the Fc region does not comprise a hinge region.

254. A method of treating cancer in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

255. A method of treating cancer in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

256. A method of treating cancer in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

257. A method of treating cancer in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

258. A method of treating cancer in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

259. A method of treating cancer in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

260. A method of treating cancer in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

261. A method of enhancing, promoting, and/or increasing the tumor infiltration of T-cells and/or NK cells in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

262. A method of enhancing, promoting, and/or increasing the tumor infiltration of T-cells and/or NK cells in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

263. A method of enhancing, promoting, and/or increasing the tumor infiltration of T-cells and/or NK cells in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

264. A method of enhancing, promoting, and/or increasing the tumor infiltration of T-cells and/or NK cells in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

265. A method of enhancing, promoting, and/or increasing the tumor infiltration of T-cells and/or NK cells in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

266. A method of enhancing, promoting, and/or increasing the tumor infiltration of T-cells and/or NK cells in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

267. A method of enhancing, promoting, and/or increasing the tumor infiltration of T-cells and/or NK cells in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

268. A method of enhancing, promoting, and/or increasing the tumor infiltration of T-cells and/or NK cells in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

269. A method of enhancing, promoting, and/or increasing the tumor infiltration of T-cells and/or NK cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the two or more doses are administered at least two weeks apart, and

wherein:
a. at least 5 amino acids are truncated from the C-terminus of the FLT3L extracellular domain; and/or
b. the Fc region does not comprise a hinge region.

270. A method of enhancing, promoting, and/or increasing the tumor infiltration of T-cells and/or NK cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the two or more doses are administered between 2 to 5 weeks apart, and

wherein:
a. at least 5 amino acids are truncated from the C-terminus of the FLT3L extracellular domain; and/or
b. the Fc region does not comprise a hinge region.

271. A method of enhancing, promoting, and/or increasing the tumor infiltration of T-cells and/or NK cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the two or more doses are administered at least about 2 weeks apart over a duration of at least about 1 month, and

wherein:
a. at least 5 amino acids are truncated from the C-terminus of the FLT3L extracellular domain; and/or
b. the Fc region does not comprise a hinge region.

272. A method of enhancing, promoting, and/or increasing the tumor infiltration of T-cells and/or NK cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the two or more doses are administered between about 2 to 5 weeks apart over a duration of between about 1 to 4 months, and

wherein:
a. at least 5 amino acids are truncated from the C-terminus of the FLT3L extracellular domain; and/or
b. the Fc region does not comprise a hinge region.

273. A method of enhancing, promoting, and/or increasing the tumor infiltration of T-cells and/or NK cells in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the doses are administered between about 2 to 5 weeks apart over a duration of between about 1 to 4 months, and

wherein:
a. at least 5 amino acids are truncated from the C-terminus of the FLT3L extracellular domain; and/or
b. the Fc region does not comprise a hinge region.

274. A method of enhancing, promoting, and/or increasing the tumor infiltration of T-cells and/or NK cells in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

275. A method of enhancing, promoting, and/or increasing the tumor infiltration of T-cells and/or NK cells in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

276. A method of enhancing, promoting, and/or increasing the tumor infiltration of T-cells and/or NK cells in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

277. A method of enhancing, promoting, and/or increasing the tumor infiltration of T-cells and/or NK cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

278. A method of enhancing, promoting, and/or increasing the tumor infiltration of T-cells and/or NK cells in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

279. A method of enhancing, promoting, and/or increasing the tumor infiltration of T-cells and/or NK cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

280. A method of enhancing, promoting, and/or increasing the tumor infiltration of T-cells and/or NK cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

281. A method of enhancing, promoting, and/or accelerating the recovery from or reversing the effects of lymphopenia in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

282. A method of enhancing, promoting, and/or accelerating the recovery from or reversing the effects of lymphopenia in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

283. A method of enhancing, promoting, and/or accelerating the recovery from or reversing the effects of lymphopenia in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

284. A method of enhancing, promoting, and/or accelerating the recovery from or reversing the effects of lymphopenia in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

285. A method of enhancing, promoting, and/or accelerating the recovery from or reversing the effects of lymphopenia in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

286. A method of enhancing, promoting, and/or accelerating the recovery from or reversing the effects of lymphopenia in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

287. A method of enhancing, promoting, and/or accelerating the recovery from or reversing the effects of lymphopenia in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

288. A method of enhancing, promoting, and/or accelerating the recovery from or reversing the effects of lymphopenia in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

289. A method of enhancing, promoting, and/or accelerating the recovery from or reversing the effects of lymphopenia in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the two or more doses are administered at least two weeks apart, and

wherein:
a. at least 5 amino acids are truncated from the C-terminus of the FLT3L extracellular domain; and/or
b. the Fc region does not comprise a hinge region.

290. A method of enhancing, promoting, and/or accelerating the recovery from or reversing the effects of lymphopenia in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the two or more doses are administered between 2 to 5 weeks apart, and

wherein:
a. at least 5 amino acids are truncated from the C-terminus of the FLT3L extracellular domain; and/or
b. the Fc region does not comprise a hinge region.

291. A method of enhancing, promoting, and/or accelerating the recovery from or reversing the effects of lymphopenia in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the two or more doses are administered at least about 2 weeks apart over a duration of at least about 1 month, and

wherein:
a. at least 5 amino acids are truncated from the C-terminus of the FLT3L extracellular domain; and/or
b. the Fc region does not comprise a hinge region.

292. A method of enhancing, promoting, and/or accelerating the recovery from or reversing the effects of lymphopenia in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the two or more doses are administered between about 2 to 5 weeks apart over a duration of between about 1 to 4 months, and

wherein:
a. at least 5 amino acids are truncated from the C-terminus of the FLT3L extracellular domain; and/or
b. the Fc region does not comprise a hinge region.

293. A method of enhancing, promoting, and/or accelerating the recovery from or reversing the effects of lymphopenia in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein at least two of the doses are administered between about 2 to 5 weeks apart over a duration of between about 1 to 4 months, and

wherein:
a. at least 5 amino acids are truncated from the C-terminus of the FLT3L extracellular domain; and/or
b. the Fc region does not comprise a hinge region.

294. A method of enhancing, promoting, and/or accelerating the recovery from or reversing the effects of lymphopenia in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

295. A method of enhancing, promoting, and/or accelerating the recovery from or reversing the effects of lymphopenia in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

296. A method of enhancing, promoting, and/or accelerating the recovery from or reversing the effects of lymphopenia in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

297. A method of enhancing, promoting, and/or accelerating the recovery from or reversing the effects of lymphopenia in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

298. A method of enhancing, promoting, and/or accelerating the recovery from or reversing the effects of lymphopenia in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

299. A method of enhancing, promoting, and/or accelerating the recovery from or reversing the effects of lymphopenia in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

300. A method of enhancing, promoting, and/or accelerating the recovery from or reversing the effects of lymphopenia in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

301. A method of enhancing, improving, and/or increasing the response to an anticancer therapy in a subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of an anticancer agent, wherein:
a. at least 5 amino acids are truncated from the C-terminus of the FLT3L extracellular domain; and/or
b. the Fc region does not comprise a hinge region.

302. A method of enhancing, improving, and/or increasing the response to an anticancer therapy in a subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of an anticancer agent, wherein:
a. at least 5 amino acids are truncated from the C-terminus of the FLT3L extracellular domain; and/or
b. the Fc region does not comprise a hinge region.

303. A method of enhancing, improving, and/or increasing the response to an anticancer therapy in a subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of an anticancer agent, wherein:
a. at least 5 amino acids are truncated from the C-terminus of the FLT3L extracellular domain; and/or
b. the Fc region does not comprise a hinge region.

304. A method of enhancing, improving, and/or increasing the response to an anticancer therapy in a subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of an anticancer agent, wherein:
a. at least 5 amino acids are truncated from the C-terminus of the FLT3L extracellular domain; and/or
b. the Fc region does not comprise a hinge region.

305. A method of enhancing, improving, and/or increasing the response to an anticancer therapy in a human subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of an anticancer agent, wherein:
a. at least 5 amino acids are truncated from the C-terminus of the FLT3L extracellular domain; and/or
b. the Fc region does not comprise a hinge region.

306. A method of enhancing, improving, and/or increasing the response to an anticancer therapy in a human subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of an anticancer agent, wherein:
a. at least 5 amino acids are truncated from the C-terminus of the FLT3L extracellular domain; and/or
b. the Fc region does not comprise a hinge region.

307. A method of enhancing, improving, and/or increasing the response to an anticancer therapy in a human subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of an anticancer agent, wherein:
a. at least 5 amino acids are truncated from the C-terminus of the FLT3L extracellular domain; and/or
b. the Fc region does not comprise a hinge region.

308. A method of enhancing, improving, and/or increasing the response to an anticancer therapy in a human subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of an anticancer agent, wherein:
a. at least 5 amino acids are truncated from the C-terminus of the FLT3L extracellular domain; and/or
b. the Fc region does not comprise a hinge region.

309. A method of enhancing, improving, and/or increasing the response to an anticancer therapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered at least two weeks apart, and (II) an effective amount of an anticancer agent,

wherein:
a. at least 5 amino acids are truncated from the C-terminus of the FLT3L extracellular domain; and/or
b. the Fc region does not comprise a hinge region.

310. A method of enhancing, improving, and/or increasing the response to an anticancer therapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered between 2 to 5 weeks apart; and (II) an effective amount of an anticancer agent,

wherein:
a. at least 5 amino acids are truncated from the C-terminus of the FLT3L extracellular domain; and/or
b. the Fc region does not comprise a hinge region.

311. A method of enhancing, improving, and/or increasing the response to an anticancer therapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered at least about 2 weeks apart over a duration of at least about 1 month; and (II) an effective amount of an anticancer agent, wherein:
a. at least 5 amino acids are truncated from the C-terminus of the FLT3L extracellular domain; and/or
b. the Fc region does not comprise a hinge region.

312. A method of enhancing, improving, and/or increasing the response to an anticancer therapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered between about 2 to 5 weeks apart over a duration of between about 1 to 4 months; and (II) an effective amount of an anticancer agent, wherein:
a. at least 5 amino acids are truncated from the C-terminus of the FLT3L extracellular domain; and/or
b. the Fc region does not comprise a hinge region.

313. A method of enhancing, improving, and/or increasing the response to an anticancer therapy in a subject in need thereof, comprising co-administering to the subject (I) between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the doses are administered between about 2 to 5 weeks apart over a duration of between about 1 to 4 months; and (II) an effective amount of an anticancer agent, wherein:
a. at least 5 amino acids are truncated from the C-terminus of the FLT3L extracellular domain; and/or
b. the Fc region does not comprise a hinge region. 314. A method of enhancing, improving, and/or increasing the response to an anticancer therapy in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of an anticancer agent, wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

315. A method of enhancing, improving, and/or increasing the response to an anticancer therapy in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of an anticancer agent, wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L
b. the Fc region does not comprise a hinge region.

316. A method of enhancing, improving, and/or increasing the response to an anticancer therapy in a subject in need thereof, comprising co-administering to the subject (I) between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of an anticancer agent, wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

317. A method of enhancing, improving, and/or increasing the response to an anticancer therapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of an anticancer agent, wherein administering the fusion protein comprises:
A. administering to the subject two or more doses of the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

318. A method of enhancing, improving, and/or increasing the response to an anticancer therapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of an anticancer agent, wherein administering the fusion protein comprises:
A. administering to the subject two or more doses of the fusion protein, wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

319. A method of enhancing, improving, and/or increasing the response to an anticancer therapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of an anticancer agent, wherein administering the fusion protein comprises:
A. administering to the subject two or more doses of an effective amount the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

320. A method of enhancing, improving, and/or increasing the response to an anticancer therapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of an anticancer agent, wherein administering the fusion protein comprises:
A. administering to the subject two or more doses of an effective amount the fusion protein, wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

321. A method of enhancing, improving, and/or increasing the response to sacituzumab govitecan in a subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of sacituzumab govitecan, wherein:
a. at least 5 amino acids are truncated from the C-terminus of the FLT3L extracellular domain; and/or
b. the Fc region does not comprise a hinge region.

322. A method of enhancing, improving, and/or increasing the response to sacituzumab govitecan in a subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of sacituzumab govitecan, wherein:
a. at least 5 amino acids are truncated from the C-terminus of the FLT3L extracellular domain; and/or
b. the Fc region does not comprise a hinge region.

323. A method of enhancing, improving, and/or increasing the response to sacituzumab govitecan in a subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of sacituzumab govitecan, wherein:
a. at least 5 amino acids are truncated from the C-terminus of the FLT3L extracellular domain; and/or
b. the Fc region does not comprise a hinge region.

324. A method of enhancing, improving, and/or increasing the response to sacituzumab govitecan in a subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of sacituzumab govitecan, wherein:
a. at least 5 amino acids are truncated from the C-terminus of the FLT3L extracellular domain; and/or
b. the Fc region does not comprise a hinge region.

325. A method of enhancing, improving, and/or increasing the response to sacituzumab govitecan in a human subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of sacituzumab govitecan, wherein:
a. at least 5 amino acids are truncated from the C-terminus of the FLT3L extracellular domain; and/or
b. the Fc region does not comprise a hinge region.

326. A method of enhancing, improving, and/or increasing the response to sacituzumab govitecan in a human subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of sacituzumab govitecan, wherein:
a. at least 5 amino acids are truncated from the C-terminus of the FLT3L extracellular domain; and/or
b. the Fc region does not comprise a hinge region.

327. A method of enhancing, improving, and/or increasing the response to sacituzumab govitecan in a human subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of sacituzumab govitecan, wherein:
a. at least 5 amino acids are truncated from the C-terminus of the FLT3L extracellular domain; and/or
b. the Fc region does not comprise a hinge region.

328. A method of enhancing, improving, and/or increasing the response to sacituzumab govitecan in a human subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of sacituzumab govitecan, wherein:
a. at least 5 amino acids are truncated from the C-terminus of the FLT3L extracellular domain; and/or
b. the Fc region does not comprise a hinge region.

329. A method of enhancing, improving, and/or increasing the response to sacituzumab govitecan in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered at least two weeks apart, and (II) an effective amount of sacituzumab govitecan,

wherein:
a. at least 5 amino acids are truncated from the C-terminus of the FLT3L extracellular domain; and/or
b. the Fc region does not comprise a hinge region.

330. A method of enhancing, improving, and/or increasing the response to sacituzumab govitecan in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered between 2 to 5 weeks apart; and (II) an effective amount of sacituzumab govitecan,

wherein:
a. at least 5 amino acids are truncated from the C-terminus of the FLT3L extracellular domain; and/or
b. the Fc region does not comprise a hinge region.

331. A method of enhancing, improving, and/or increasing the response to sacituzumab govitecan in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered at least about 2 weeks apart over a duration of at least about 1 month; and (II) an effective amount of sacituzumab govitecan, wherein:
a. at least 5 amino acids are truncated from the C-terminus of the FLT3L extracellular domain; and/or
b. the Fc region does not comprise a hinge region.

332. A method of enhancing, improving, and/or increasing the response to sacituzumab govitecan in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered between about 2 to 5 weeks apart over a duration of between about 1 to 4 months; and (II) an effective amount of sacituzumab govitecan,

wherein:
a. at least 5 amino acids are truncated from the C-terminus of the FLT3L extracellular domain; and/or
b. the Fc region does not comprise a hinge region.

333. A method of enhancing, improving, and/or increasing the response to sacituzumab govitecan in a subject in need thereof, comprising co-administering to the subject (I) between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the doses are administered between about 2 to 5 weeks apart over a duration of between about 1 to 4 months; and (II) an effective amount of sacituzumab govitecan,

wherein:
a. at least 5 amino acids are truncated from the C-terminus of the FLT3L extracellular domain; and/or
b. the Fc region does not comprise a hinge region.

334. A method of enhancing, improving, and/or increasing the response to sacituzumab govitecan in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of sacituzumab govitecan, wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

335. A method of enhancing, improving, and/or increasing the response to sacituzumab govitecan in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of sacituzumab govitecan, wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

336. A method of enhancing, improving, and/or increasing the response to sacituzumab govitecan in a subject in need thereof, comprising co-administering to the subject (I) between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of sacituzumab govitecan, wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

337. A method of enhancing, improving, and/or increasing the response to sacituzumab govitecan in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of sacituzumab govitecan, wherein administering the fusion protein comprises:
A. administering to the subject two or more doses of the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

338. A method of enhancing, improving, and/or increasing the response to sacituzumab govitecan in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of sacituzumab govitecan, wherein administering the fusion protein comprises:
A. administering to the subject two or more doses of the fusion protein, wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

339. A method of enhancing, improving, and/or increasing the response to sacituzumab govitecan in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of sacituzumab govitecan, wherein administering the fusion protein comprises:
A. administering to the subject two or more doses of an effective amount the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

340. A method of enhancing, improving, and/or increasing the response to sacituzumab govitecan in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of sacituzumab govitecan, wherein administering the fusion protein comprises:
A. administering to the subject two or more doses of an effective amount the fusion protein, wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

341. A method of enhancing, improving, and/or increasing the response to magrolimab in a subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of magrolimab, wherein:
a. at least 5 amino acids are truncated from the C-terminus of the FLT3L extracellular domain; and/or
b. the Fc region does not comprise a hinge region.

342. A method of enhancing, improving, and/or increasing the response to magrolimab in a subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of magrolimab, wherein:
a. at least 5 amino acids are truncated from the C-terminus of the FLT3L extracellular domain; and/or
b. the Fc region does not comprise a hinge region.

343. A method of enhancing, improving, and/or increasing the response to magrolimab in a subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of magrolimab, wherein:
a. at least 5 amino acids are truncated from the C-terminus of the FLT3L extracellular domain; and/or
b. the Fc region does not comprise a hinge region.

344. A method of enhancing, improving, and/or increasing the response to magrolimab in a subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of magrolimab, wherein:
a. at least 5 amino acids are truncated from the C-terminus of the FLT3L extracellular domain; and/or
b. the Fc region does not comprise a hinge region.

345. A method of enhancing, improving, and/or increasing the response to magrolimab in a human subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of magrolimab, wherein:
a. at least 5 amino acids are truncated from the C-terminus of the FLT3L extracellular domain; and/or
b. the Fc region does not comprise a hinge region.

346. A method of enhancing, improving, and/or increasing the response to magrolimab in a human subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of magrolimab, wherein:
a. at least 5 amino acids are truncated from the C-terminus of the FLT3L extracellular domain; and/or
b. the Fc region does not comprise a hinge region.

347. A method of enhancing, improving, and/or increasing the response to magrolimab in a human subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of magrolimab, wherein:
a. at least 5 amino acids are truncated from the C-terminus of the FLT3L extracellular domain; and/or
b. the Fc region does not comprise a hinge region.

348. A method of enhancing, improving, and/or increasing the response to magrolimab in a human subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of magrolimab, wherein:
a. at least 5 amino acids are truncated from the C-terminus of the FLT3L extracellular domain; and/or
b. the Fc region does not comprise a hinge region.

349. A method of enhancing, improving, and/or increasing the response to magrolimab in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered at least two weeks apart, and (II) an effective amount of magrolimab, wherein:
a. at least 5 amino acids are truncated from the C-terminus of the FLT3L extracellular domain; and/or
b. the Fc region does not comprise a hinge region.

350. A method of enhancing, improving, and/or increasing the response to magrolimab in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered between 2 to 5 weeks apart; and (II) an effective amount of magrolimab, wherein:
a. at least 5 amino acids are truncated from the C-terminus of the FLT3L extracellular domain; and/or
b. the Fc region does not comprise a hinge region.

351. A method of enhancing, improving, and/or increasing the response to magrolimab in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered at least about 2 weeks apart over a duration of at least about 1 month; and (II) an effective amount of magrolimab, wherein:
a. at least 5 amino acids are truncated from the C-terminus of the FLT3L extracellular domain; and/or
b. the Fc region does not comprise a hinge region.

352. A method of enhancing, improving, and/or increasing the response to magrolimab in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered between about 2 to 5 weeks apart over a duration of between about 1 to 4 months; and (II) an effective amount of magrolimab,

wherein:
a. at least 5 amino acids are truncated from the C-terminus of the FLT3L extracellular domain; and/or
b. the Fc region does not comprise a hinge region.

353. A method of enhancing, improving, and/or increasing the response to magrolimab in a subject in need thereof, comprising co-administering to the subject (I) between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the doses are administered between about 2 to 5 weeks apart over a duration of between about 1 to 4 months; and (II) an effective amount of magrolimab, wherein:
a. at least 5 amino acids are truncated from the C-terminus of the FLT3L extracellular domain; and/or
b. the Fc region does not comprise a hinge region.

354. A method of enhancing, improving, and/or increasing the response to magrolimab in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and (II) an effective amount of magrolimab, wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

355. A method of enhancing, improving, and/or increasing the response to magrolimab in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of magrolimab, wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

356. A method of enhancing, improving, and/or increasing the response to magrolimab in a subject in need thereof, comprising co-administering to the subject (I) between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of magrolimab, wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

357. A method of enhancing, improving, and/or increasing the response to magrolimab in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of magrolimab, wherein administering the fusion protein comprises:
A. administering to the subject two or more doses of the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

358. A method of enhancing, improving, and/or increasing the response to magrolimab in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of magrolimab, wherein administering the fusion protein comprises:
A. administering to the subject two or more doses of the fusion protein, wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

359. A method of enhancing, improving, and/or increasing the response to magrolimab in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of magrolimab, wherein administering the fusion protein comprises:
A. administering to the subject two or more doses of an effective amount the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

360. A method of enhancing, improving, and/or increasing the response to magrolimab in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of magrolimab, wherein administering the fusion protein comprises:
A. administering to the subject two or more doses of an effective amount the fusion protein, wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

361. A method of enhancing, improving, and/or increasing the response to an inhibitor of MCL-1 in a subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the MCL-1 inhibitor, wherein:
a. at least 5 amino acids are truncated from the C-terminus of the FLT3L extracellular domain; and/or
b. the Fc region does not comprise a hinge region.

362. A method of enhancing, improving, and/or increasing the response to an inhibitor of MCL-1 in a subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the MCL-1 inhibitor, wherein:
a. at least 5 amino acids are truncated from the C-terminus of the FLT3L extracellular domain; and/or
b. the Fc region does not comprise a hinge region.

363. A method of enhancing, improving, and/or increasing the response to an inhibitor of MCL-1 in a subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the MCL-1 inhibitor, wherein:
a. at least 5 amino acids are truncated from the C-terminus of the FLT3L extracellular domain; and/or
b. the Fc region does not comprise a hinge region.

364. A method of enhancing, improving, and/or increasing the response to an inhibitor of MCL-1 in a subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the MCL-1 inhibitor, wherein:
a. at least 5 amino acids are truncated from the C-terminus of the FLT3L extracellular domain; and/or
b. the Fc region does not comprise a hinge region.

365. A method of enhancing, improving, and/or increasing the response to an inhibitor of MCL-1 in a human subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the MCL-1 inhibitor, wherein:
a. at least 5 amino acids are truncated from the C-terminus of the FLT3L extracellular domain; and/or
b. the Fc region does not comprise a hinge region.

366. A method of enhancing, improving, and/or increasing the response to an inhibitor of MCL-1 in a human subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the MCL-1 inhibitor, wherein:
a. at least 5 amino acids are truncated from the C-terminus of the FLT3L extracellular domain; and/or
b. the Fc region does not comprise a hinge region.

367. A method of enhancing, improving, and/or increasing the response to an inhibitor of MCL-1 in a human subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the MCL-1 inhibitor, wherein:
a. at least 5 amino acids are truncated from the C-terminus of the FLT3L extracellular domain; and/or
b. the Fc region does not comprise a hinge region.

368. A method of enhancing, improving, and/or increasing the response to an inhibitor of MCL-1 in a human subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the MCL-1 inhibitor, wherein:
a. at least 5 amino acids are truncated from the C-terminus of the FLT3L extracellular domain; and/or
b. the Fc region does not comprise a hinge region.

369. A method of enhancing, improving, and/or increasing the response to an inhibitor of MCL-1 in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered at least two weeks apart, and (II) an effective amount of the MCL-1 inhibitor, wherein:
a. at least 5 amino acids are truncated from the C-terminus of the FLT3L extracellular domain; and/or
b. the Fc region does not comprise a hinge region.

370. A method of enhancing, improving, and/or increasing the response to an inhibitor of MCL-1 in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered between 2 to 5 weeks apart; and (II) an effective amount of the MCL-1 inhibitor, wherein:
a. at least 5 amino acids are truncated from the C-terminus of the FLT3L extracellular domain; and/or
b. the Fc region does not comprise a hinge region.

371. A method of enhancing, improving, and/or increasing the response to an inhibitor of MCL-1 in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered at least about 2 weeks apart over a duration of at least about 1 month; and (II) an effective amount of the MCL-1 inhibitor, wherein:
a. at least 5 amino acids are truncated from the C-terminus of the FLT3L extracellular domain; and/or
b. the Fc region does not comprise a hinge region.

372. A method of enhancing, improving, and/or increasing the response to an inhibitor of MCL-1 in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered between about 2 to 5 weeks apart over a duration of between about 1 to 4 months; and (II) an effective amount of the MCL-1 inhibitor, wherein:
a. at least 5 amino acids are truncated from the C-terminus of the FLT3L extracellular domain; and/or
b. the Fc region does not comprise a hinge region.

373. A method of enhancing, improving, and/or increasing the response to an inhibitor of MCL-1 in a subject in need thereof, comprising co-administering to the subject (I) between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the doses are administered between about 2 to 5 weeks apart over a duration of between about 1 to 4 months; and (II) an effective amount of the MCL-1 inhibitor, wherein:
a. at least 5 amino acids are truncated from the C-terminus of the FLT3L extracellular domain; and/or
b. the Fc region does not comprise a hinge region.

374. A method of enhancing, improving, and/or increasing the response to an inhibitor of MCL-1 in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the MCL-1 inhibitor, wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

375. A method of enhancing, improving, and/or increasing the response to an inhibitor of MCL-1 in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the MCL-1 inhibitor, wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

376. A method of enhancing, improving, and/or increasing the response to an inhibitor of MCL-1 in a subject in need thereof, comprising co-administering to the subject (I) between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the MCL-1 inhibitor, wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

377. A method of enhancing, improving, and/or increasing the response to an inhibitor of MCL-1 in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the MCL-1 inhibitor, wherein administering the fusion protein comprises:
A. administering to the subject two or more doses of the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

378. A method of enhancing, improving, and/or increasing the response to an inhibitor of MCL-1 in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the MCL-1 inhibitor, wherein administering the fusion protein comprises:
A. administering to the subject two or more doses of the fusion protein, wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

379. A method of enhancing, improving, and/or increasing the response to an inhibitor of MCL-1 in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the MCL-1 inhibitor, wherein administering the fusion protein comprises:
A. administering to the subject two or more doses of an effective amount the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

380. A method of enhancing, improving, and/or increasing the response to an inhibitor of MCL-1 in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the MCL-1 inhibitor, wherein administering the fusion protein comprises:
A. administering to the subject two or more doses of an effective amount the fusion protein, wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

381. A method of promoting, inducing and/or increasing the expansion and/or proliferation of a cell or a population of cells that express fms related tyrosine kinase 3 (FLT3, CD135) in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the expansion and/or proliferation of the cell or population of cells is observed within 5 to 15 days after administration of the fusion protein, and wherein:
a. at least 5 amino acids are truncated from the C-terminus of the FLT3L extracellular domain; and/or
b. the Fc region does not comprise a hinge region.

382. A method of promoting, inducing and/or increasing the expansion and/or proliferation of a cell or a population of cells that express fms related tyrosine kinase 3 (FLT3, CD135) in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the expansion and/or proliferation of the cell or population of cells is observed within 5 to 15 days after administration of the fusion protein, wherein:
a. at least 5 amino acids are truncated from the C-terminus of the FLT3L extracellular domain; and/or
b. the Fc region does not comprise a hinge region.

383. A method of promoting, inducing and/or increasing the expansion and/or proliferation of a cell or a population of cells that express fms related tyrosine kinase 3 (FLT3, CD135) in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the expansion and/or proliferation of the cell or population of cells is observed within 5 to 15 days after administration of the fusion protein, wherein:
a. at least 5 amino acids are truncated from the C-terminus of the FLT3L extracellular domain; and/or
b. the Fc region does not comprise a hinge region.

384. A method of promoting, inducing and/or increasing the expansion and/or proliferation of a cell or a population of cells that express fms related tyrosine kinase 3 (FLT3, CD135) in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the expansion and/or proliferation of the cell or population of cells is observed within 5 to 15 days after administration of the fusion protein, wherein:
a. at least 5 amino acids are truncated from the C-terminus of the FLT3L extracellular domain; and/or
b. the Fc region does not comprise a hinge region.

385. A method of promoting, inducing and/or increasing the expansion and/or proliferation of a cell or a population of cells that express fms related tyrosine kinase 3 (FLT3, CD135) in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the expansion and/or proliferation of the cell or population of cells is observed within 5 to 15 days after administration of the fusion protein, and wherein:
a. at least 5 amino acids are truncated from the C-terminus of the FLT3L extracellular domain; and/or
b. the Fc region does not comprise a hinge region.

386. A method of promoting, inducing and/or increasing the expansion and/or proliferation of a cell or a population of cells that express fms related tyrosine kinase 3 (FLT3, CD135) in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the expansion and/or proliferation of the cell or population of cells is observed within 5 to 15 days after administration of the fusion protein, wherein:
a. at least 5 amino acids are truncated from the C-terminus of the FLT3L extracellular domain; and/or
b. the Fc region does not comprise a hinge region.

387. A method of promoting, inducing and/or increasing the expansion and/or proliferation of a cell or a population of cells that express fms related tyrosine kinase 3 (FLT3, CD135) in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the expansion and/or proliferation of the cell or population of cells is observed within 5 to 15 days after administration of the fusion protein, wherein:
a. at least 5 amino acids are truncated from the C-terminus of the FLT3L extracellular domain; and/or
b. the Fc region does not comprise a hinge region.

388. A method of promoting, inducing and/or increasing the expansion and/or proliferation of a cell or a population of cells that express fms related tyrosine kinase 3 (FLT3, CD135) in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the expansion and/or proliferation of the cell or population of cells is observed within 5 to 15 days after administration of the fusion protein, wherein:
a. at least 5 amino acids are truncated from the C-terminus of the FLT3L extracellular domain; and/or
b. the Fc region does not comprise a hinge region.

389. A method of promoting, inducing and/or increasing the expansion and/or proliferation of a cell or a population of cells that express fms related tyrosine kinase 3 (FLT3, CD135) in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least two of the two or more doses are administered at least two weeks apart; and
b. the expansion and/or proliferation of the cell or population of cells is observed within 5 to 15 days after administration of the fusion protein, and
   wherein:
a. at least 5 amino acids are truncated from the C-terminus of the FLT3L extracellular domain; and/or
b. the Fc region does not comprise a hinge region.

390. A method of promoting, inducing and/or increasing the expansion and/or proliferation of a cell or a population of cells that express fms related tyrosine kinase 3 (FLT3, CD135) in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least two of the two or more doses are administered between 2 to 5 weeks apart; and
b. the expansion and/or proliferation of the cell or population of cells is observed within 5 to 15 days after administration of the fusion protein, and
   wherein:
a. at least 5 amino acids are truncated from the C-terminus of the FLT3L extracellular domain; and/or
b. the Fc region does not comprise a hinge region.

391. A method of promoting, inducing and/or increasing the expansion and/or proliferation of a cell or a population of cells that express fms related tyrosine kinase 3 (FLT3, CD135) in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least two of the two or more doses are administered at least about 2 weeks apart over a duration of at least about 1 month; and
b. the expansion and/or proliferation of the cell or population of cells is observed within 5 to 15 days after administration of the fusion protein, and
   wherein:
a. at least 5 amino acids are truncated from the C-terminus of the FLT3L extracellular domain; and/or
b. the Fc region does not comprise a hinge region.

392. A method of promoting, inducing and/or increasing the expansion and/or proliferation of a cell or a population of cells that express fms related tyrosine kinase 3 (FLT3, CD135) in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least two of the two or more doses are administered between about 2 to 5 weeks apart over a duration of between about 1 to 4 months; and
b. the expansion and/or proliferation of the cell or population of cells is observed within 5 to 15 days after administration of the fusion protein, and
   wherein:
a. at least 5 amino acids are truncated from the C-terminus of the FLT3L extracellular domain; and/or
b. the Fc region does not comprise a hinge region.

393. A method of promoting, inducing and/or increasing the expansion and/or proliferation of a cell or a population of cells that express fms related tyrosine kinase 3 (FLT3, CD135) in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least two of the doses are administered between about 2 to 5 weeks apart over a duration of between about 1 to 4 months; and
b. the expansion and/or proliferation of the cell or population of cells is observed within 5 to 15 days after administration of the fusion protein, and
   wherein:
a. at least 5 amino acids are truncated from the C-terminus of the FLT3L extracellular domain; and/or
b. the Fc region does not comprise a hinge region.

394. A method of promoting, inducing and/or increasing the expansion and/or proliferation of a cell or a population of cells that express fms related tyrosine kinase 3 (FLT3, CD135) in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. each dose comprises between about 200 μg to about 30000 μg of the fusion protein;
b. at least two of the two or more doses are administered at least 2 weeks apart over a duration of at least 1 month; and
c. the expansion and/or proliferation of the cell or population of cells is observed within 5 to 15 days after administration of the fusion protein, and
   wherein:
a. at least 5 amino acids are truncated from the C-terminus of the FLT3L extracellular domain; and/or
b. the Fc region does not comprise a hinge region.

395. A method of promoting, inducing and/or increasing the expansion and/or proliferation of a cell or a population of cells that express fms related tyrosine kinase 3 (FLT3, CD135) in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. each dose comprises between about 200 μg to about 30000 μg of the fusion protein;
b. at least two of the two or more doses are administered between about 2 to 5 weeks apart over a duration of between about 1 to 4 months; and
c. the expansion and/or proliferation of the cell or population of cells is observed within 5 to 15 days after administration of the fusion protein, and
   wherein:
a. at least 5 amino acids are truncated from the C-terminus of the FLT3L extracellular domain; and/or
b. the Fc region does not comprise a hinge region.

396. A method of promoting, inducing and/or increasing the expansion and/or proliferation of a cell or a population of cells that express fms related tyrosine kinase 3 (FLT3, CD135) in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. each dose comprises between about 200 μg to about 30000 μg of the fusion protein;
b. at least two of the doses are administered between about 2 to 5 weeks apart over a duration of between about 1 to 4 months; and
c. the expansion and/or proliferation of the cell or population of cells is observed within 5 to 15 days after administration of the fusion protein, and
   wherein:
a. at least 5 amino acids are truncated from the C-terminus of the FLT3L extracellular domain; and/or
b. the Fc region does not comprise a hinge region.

397. A method of promoting, inducing and/or increasing the expansion and/or proliferation of a cell or a population of cells that express fms related tyrosine kinase 3 (FLT3, CD135) in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
   i. the dosing interval for the two or more doses is one dose every 2 to 4 weeks; and
   ii. the expansion and/or proliferation of the cell or population of cells is observed within 5 to 15 days after administration of the fusion protein, and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

398. A method of promoting, inducing and/or increasing the expansion and/or proliferation of a cell or a population of cells that express fms related tyrosine kinase 3 (FLT3, CD135) in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
   i. each dose comprises between about 200 μg to about 30000 μg of the fusion protein;
   ii. the dosing interval for the two or more doses is once every 2 to 4 weeks; and
   iii. the expansion and/or proliferation of the cell or population of cells is observed within 5 to 15 days after administration of the fusion protein, and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

399. A method of promoting, inducing and/or increasing the expansion and/or proliferation of a cell or a population of cells that express fms related tyrosine kinase 3 (FLT3, CD135) in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
   i. the dosing interval for the two or more doses is one dose every 2 to 4 weeks; and
   ii. the expansion and/or proliferation of the cell or population of cells is observed within 5 to 15 days after administration of the fusion protein; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

400. A method of promoting, inducing and/or increasing the expansion and/or proliferation of a cell or a population of cells that express fms related tyrosine kinase 3 (FLT3, CD135) in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
   i. the two or more doses are administered about 8 to 20 days apart; and
   ii. the expansion and/or proliferation of the cell or population of cells is observed within 5 to 15 days after administration of the fusion protein,
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

401. A method of enhancing, improving, and/or increasing the response to an immunotherapy in a subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the immunotherapy, wherein:
a. at least 5 amino acids are truncated from the C-terminus of the FLT3L extracellular domain; and/or
b. the Fc region does not comprise a hinge region.

402. A method of enhancing, improving, and/or increasing the response to an immunotherapy in a subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the immunotherapy, wherein:
a. at least 5 amino acids are truncated from the C-terminus of the FLT3L extracellular domain; and/or
b. the Fc region does not comprise a hinge region.

403. A method of enhancing, improving, and/or increasing the response to an immunotherapy in a subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the immunotherapy, wherein:
a. at least 5 amino acids are truncated from the C-terminus of the FLT3L extracellular domain; and/or
b. the Fc region does not comprise a hinge region.

404. A method of enhancing, improving, and/or increasing the response to an immunotherapy in a subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the immunotherapy, wherein:
a. at least 5 amino acids are truncated from the C-terminus of the FLT3L extracellular domain; and/or
b. the Fc region does not comprise a hinge region.

405. A method of enhancing, improving, and/or increasing the response to an immunotherapy in a human subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the immunotherapy, wherein:
a. at least 5 amino acids are truncated from the C-terminus of the FLT3L extracellular domain; and/or
b. the Fc region does not comprise a hinge region.

406. A method of enhancing, improving, and/or increasing the response to an immunotherapy in a human subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the immunotherapy, wherein:
a. at least 5 amino acids are truncated from the C-terminus of the FLT3L extracellular domain; and/or
b. the Fc region does not comprise a hinge region.

407. A method of enhancing, improving, and/or increasing the response to an immunotherapy in a human subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the immunotherapy, wherein:
a. at least 5 amino acids are truncated from the C-terminus of the FLT3L extracellular domain; and/or
b. the Fc region does not comprise a hinge region.

408. A method of enhancing, improving, and/or increasing the response to an immunotherapy in a human subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the immunotherapy, wherein:
a. at least 5 amino acids are truncated from the C-terminus of the FLT3L extracellular domain; and/or
b. the Fc region does not comprise a hinge region.

409. A method of enhancing, improving, and/or increasing the response to an immunotherapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered at least two weeks apart, and (II) an effective amount of the immunotherapy,

wherein:
a. at least 5 amino acids are truncated from the C-terminus of the FLT3L extracellular domain; and/or
b. the Fc region does not comprise a hinge region.

410. A method of enhancing, improving, and/or increasing the response to an immunotherapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered between 2 to 5 weeks apart; and (II) an effective amount of the immunotherapy, wherein:
a. at least 5 amino acids are truncated from the C-terminus of the FLT3L extracellular domain; and/or
b. the Fc region does not comprise a hinge region.

411. A method of enhancing, improving, and/or increasing the response to an immunotherapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered at least about 2 weeks apart over a duration of at least about 1 month; and (II) an effective amount of the immunotherapy, wherein:
a. at least 5 amino acids are truncated from the C-terminus of the FLT3L extracellular domain; and/or
b. the Fc region does not comprise a hinge region.

412. A method of enhancing, improving, and/or increasing the response to an immunotherapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered between about 2 to 5 weeks apart over a duration of between about 1 to 4 months; and (II) an effective amount of the immunotherapy, wherein:
a. at least 5 amino acids are truncated from the C-terminus of the FLT3L extracellular domain; and/or
b. the Fc region does not comprise a hinge region.

413. A method of enhancing, improving, and/or increasing the response to an immunotherapy in a subject in need thereof, comprising co-administering to the subject (I) between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the doses are administered between about 2 to 5 weeks apart over a duration of between about 1 to 4 months; and (II) an effective amount of the immunotherapy, wherein:
a. at least 5 amino acids are truncated from the C-terminus of the FLT3L extracellular domain; and/or
b. the Fc region does not comprise a hinge region.

414. A method of enhancing, improving, and/or increasing the response to an immunotherapy in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the immunotherapy, wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

415. A method of enhancing, improving, and/or increasing the response to an immunotherapy in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the immunotherapy, wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

416. A method of enhancing, improving, and/or increasing the response to an immunotherapy in a subject in need thereof, comprising co-administering to the subject (I) between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the immunotherapy, wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

417. A method of enhancing, improving, and/or increasing the response to an immunotherapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the immunotherapy, wherein administering the fusion protein comprises:
A. administering to the subject two or more doses of the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

418. A method of enhancing, improving, and/or increasing the response to an immunotherapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the immunotherapy, wherein administering the fusion protein comprises:
A. administering to the subject two or more doses of the fusion protein, wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

419. A method of enhancing, improving, and/or increasing the response to an immunotherapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the immunotherapy, wherein administering the fusion protein comprises:
A. administering to the subject two or more doses of an effective amount the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months; and
   wherein:
a. at least 5 amino acids are truncated from the C-terminus of the FLT3L extracellular domain; and/or
b. the Fc region does not comprise a hinge region.

420. A method of enhancing, improving, and/or increasing the response to an immunotherapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27, and (II) an effective amount of the immunotherapy, wherein administering the fusion protein comprises:
A. administering to the subject two or more doses of an effective amount the fusion protein, wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
c. at least 5 amino acids are truncated from the C-terminus of the FLT3L extracellular domain; and/or
d. the Fc region does not comprise a hinge region.

421. A method of inducing the immune system in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

422. A method of inducing the immune system in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

423. A method of inducing the immune system in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

424. A method of inducing the immune system in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

425. A method of inducing the immune system in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

426. A method of inducing the immune system in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

427. A method of inducing the immune system in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. at least 5 amino acids are truncated from the C-terminus of the FLT3L extracellular domain; and/or
b. the Fc region does not comprise a hinge region.

428. A method of inducing the immune system in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc regionwherein:
a. at least 5 amino acids are truncated from the C-terminus of the FLT3L extracellular domain; and/or
b. the Fc region does not comprise a hinge region.

429. A method of inducing the immune system in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered at least two weeks apart;

wherein:
a. at least 5 amino acids are truncated from the C-terminus of the FLT3L extracellular domain; and/or
b. the Fc region does not comprise a hinge region.

430. A method of inducing the immune system in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered between 2 to 5 weeks apart; and

wherein:
a. at least 5 amino acids are truncated from the C-terminus of the FLT3L extracellular domain; and/or
b. the Fc region does not comprise a hinge region.

431. A method of inducing the immune system in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered at least about 2 weeks apart over a duration of at least about 1 month, and

wherein:
a. at least 5 amino acids are truncated from the C-terminus of the FLT3L extracellular domain; and/or
b. the Fc region does not comprise a hinge region.

432. A method of inducing the immune system in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the two or more doses are administered between about 2 to 5 weeks apart over a duration of between about 1 to 4 months, and

wherein:
a. at least 5 amino acids are truncated from the C-terminus of the FLT3L extracellular domain; and/or
b. the Fc region does not comprise a hinge region.

433. A method of inducing the immune system in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein at least two of the doses are administered between about 2 to 5 weeks apart over a duration of between about 1 to 4 months, and

wherein:
a. at least 5 amino acids are truncated from the C-terminus of the FLT3L extracellular domain; and/or
b. the Fc region does not comprise a hinge region.

434. A method of inducing the immune system in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. each dose comprises between about 200 μg to about 30000 μg of the fusion protein; and
b. at least two of the two or more doses are administered at least 2 weeks apart over a duration of at least 1 month, and
   wherein:
a. at least 5 amino acids are truncated from the C-terminus of the FLT3L extracellular domain; and/or
b. the Fc region does not comprise a hinge region.

435. A method of inducing the immune system in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. each dose comprises between about 200 μg to about 30000 μg of the fusion protein; and
b. at least two of the two or more doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

436. A method of inducing the immune system in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
a. each dose comprises between about 200 μg to about 30000 μg of the fusion protein; and
b. at least two of the doses are administered between about 2 to 5 weeks apart over a duration of between about 1 to 4 months, and
   wherein:
a. at least 5 amino acids are truncated from the C-terminus of the FLT3L extracellular domain; and/or
b. the Fc region does not comprise a hinge region.

437. A method of inducing the immune system in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A, and
   wherein:
a. at least 5 amino acids are truncated from the C-terminus of the FLT3L extracellular domain; and/or
b. the Fc region does not comprise a hinge region.

438. A method of inducing the immune system in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A; and
   wherein:
a. at least 5 amino acids are truncated from the C-terminus of the FLT3L extracellular domain; and/or
b. the Fc region does not comprise a hinge region.

439. A method of inducing the immune system in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months, and
   wherein:
a. at least 5 amino acids are truncated from the C-terminus of the FLT3L extracellular domain; and/or
b. the Fc region does not comprise a hinge region.

440. A method of inducing the immune system in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18 and 21-27; and wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B,
   wherein:
a. at least 5 amino acids are truncated from the C-terminus of the FLT3L extracellular domain; and/or
b. the Fc region does not comprise a hinge region.

441. A method of preventing, reducing and/or inhibiting the recurrence, growth, proliferation, migration and/or metastasis of a cancer cell or population of cancer cells in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

442. A method of preventing, reducing and/or inhibiting the recurrence, growth, proliferation, migration and/or metastasis of a cancer cell or population of cancer cells in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

443. A method of preventing, reducing and/or inhibiting the recurrence, growth, proliferation, migration and/or metastasis of a cancer cell or population of cancer cells in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

444. A method of preventing, reducing and/or inhibiting the recurrence, growth, proliferation, migration and/or metastasis of a cancer cell or population of cancer cells in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

445. A method of preventing, reducing and/or inhibiting the recurrence, growth, proliferation, migration and/or metastasis of a cancer cell or population of cancer cells in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

446. A method of preventing, reducing and/or inhibiting the recurrence, growth, proliferation, migration and/or metastasis of a cancer cell or population of cancer cells in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

447. A method of preventing, reducing and/or inhibiting the recurrence, growth, proliferation, migration and/or metastasis of a cancer cell or population of cancer cells in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

448. A method of preventing, reducing and/or inhibiting the recurrence, growth, proliferation, migration and/or metastasis of a cancer cell or population of cancer cells in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

449. A method of preventing, reducing and/or inhibiting the recurrence, growth, proliferation, migration and/or metastasis of a cancer cell or population of cancer cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the two or more doses are administered at least two weeks apart.

450. A method of preventing, reducing and/or inhibiting the recurrence, growth, proliferation, migration and/or metastasis of a cancer cell or population of cancer cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the two or more doses are administered between 2 to 5 weeks apart.

451. A method of preventing, reducing and/or inhibiting the recurrence, growth, proliferation, migration and/or metastasis of a cancer cell or population of cancer cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the two or more doses are administered at least about 2 weeks apart over a duration of at least about 1 month, and

wherein:
a. at least 5 amino acids are truncated from the C-terminus of the FLT3L extracellular domain; and/or
b. the Fc region does not comprise a hinge region.

452. A method of preventing, reducing and/or inhibiting the recurrence, growth, proliferation, migration and/or metastasis of a cancer cell or population of cancer cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the two or more doses are administered between about 2 to 5 weeks apart over a duration of between about 1 to 4 month.

453. A method of preventing, reducing and/or inhibiting the recurrence, growth, proliferation, migration and/or metastasis of a cancer cell or population of cancer cells in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the doses are administered between about 2 to 5 weeks apart over a duration of between about 1 to 4 months.

454. A method of preventing, reducing and/or inhibiting the recurrence, growth, proliferation, migration and/or metastasis of a cancer cell or population of cancer cells in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein:
a. each dose comprises between about 200 μg to about 30000 μg of the fusion protein; and
b. at least two of the two or more doses are administered at least 2 weeks apart over a duration of at least 1 month.

455. A method of preventing, reducing and/or inhibiting the recurrence, growth, proliferation, migration and/or metastasis of a cancer cell or population of cancer cells in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein:
a. each dose comprises between about 200 μg to about 30000 μg of the fusion protein; and
b. at least two of the two or more doses are administered between about 2 to 5 weeks apart over a duration of between about 1 to 4 months.

456. A method of preventing, reducing and/or inhibiting the recurrence, growth, proliferation, migration and/or metastasis of a cancer cell or population of cancer cells in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein:
a. each dose comprises between about 200 μg to about 30000 μg of the fusion protein; and
b. at least two of the doses are administered between about 2 to 5 weeks apart over a duration of between about 1 to 4 months.

457. A method of preventing, reducing and/or inhibiting the recurrence, growth, proliferation, migration and/or metastasis of a cancer cell or population of cancer cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

458. A method of preventing, reducing and/or inhibiting the recurrence, growth, proliferation, migration and/or metastasis of a cancer cell or population of cancer cells in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

459. A method of preventing, reducing and/or inhibiting the recurrence, growth, proliferation, migration and/or metastasis of a cancer cell or population of cancer cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months.

460. A method of preventing, reducing and/or inhibiting the recurrence, growth, proliferation, migration and/or metastasis of a cancer cell or population of cancer cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B.

461. A method of treating cancer in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

462. A method of treating cancer in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

463. A method of treating cancer in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

464. A method of treating cancer in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

465. A method of treating cancer in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

466. A method of treating cancer in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

467. A method of treating cancer in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

468. A method of treating cancer in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

469. A method of treating cancer in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the two or more doses are administered at least two weeks apart.

470. A method of treating cancer in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the two or more doses are administered between 2 to 5 weeks apart.

471. A method of treating cancer in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the two or more doses are administered at least about 2 weeks apart over a duration of at least about 1 month, and

wherein:
a. at least 5 amino acids are truncated from the C-terminus of the FLT3L extracellular domain; and/or
b. the Fc region does not comprise a hinge region.

472. A method of treating cancer in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the two or more doses are administered between about 2 to 5 weeks apart over a duration of between about 1 to 4 month.

473. A method of treating cancer in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the doses are administered between about 2 to 5 weeks apart over a duration of between about 1 to 4 months.

474. A method of treating cancer in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein:
a. each dose comprises between about 200 μg to about 30000 μg of the fusion protein; and
b. at least two of the two or more doses are administered at least 2 weeks apart over a duration of at least 1 month.

475. A method of treating cancer in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein:
a. each dose comprises between about 200 μg to about 30000 μg of the fusion protein; and
b. at least two of the two or more doses are administered between about 2 to 5 weeks apart over a duration of between about 1 to 4 months.

476. A method of treating cancer in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein:
a. each dose comprises between about 200 μg to about 30000 μg of the fusion protein; and
b. at least two of the doses are administered between about 2 to 5 weeks apart over a duration of between about 1 to 4 months.

477. A method of treating cancer in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

478. A method of treating cancer in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

479. A method of treating cancer in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months.

480. A method of treating cancer in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B.

481. A method of enhancing, promoting, and/or increasing the tumor infiltration of T-cells and/or NK cells in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

482. A method of enhancing, promoting, and/or increasing the tumor infiltration of T-cells and/or NK cells in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

483. A method of enhancing, promoting, and/or increasing the tumor infiltration of T-cells and/or NK cells in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

484. A method of enhancing, promoting, and/or increasing the tumor infiltration of T-cells and/or NK cells in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

485. A method of enhancing, promoting, and/or increasing the tumor infiltration of T-cells and/or NK cells in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

486. A method of enhancing, promoting, and/or increasing the tumor infiltration of T-cells and/or NK cells in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

487. A method of enhancing, promoting, and/or increasing the tumor infiltration of T-cells and/or NK cells in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

488. A method of enhancing, promoting, and/or increasing the tumor infiltration of T-cells and/or NK cells in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

489. A method of enhancing, promoting, and/or increasing the tumor infiltration of T-cells and/or NK cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the two or more doses are administered at least two weeks apart.

490. A method of enhancing, promoting, and/or increasing the tumor infiltration of T-cells and/or NK cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the two or more doses are administered between 2 to 5 weeks apart.

491. A method of enhancing, promoting, and/or increasing the tumor infiltration of T-cells and/or NK cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the two or more doses are administered at least about 2 weeks apart over a duration of at least about 1 month.

492. A method of enhancing, promoting, and/or increasing the tumor infiltration of T-cells and/or NK cells in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the two or more doses are administered between about 2 to 5 weeks apart over a duration of between about 1 to 4 months.

493. A method of enhancing, promoting, and/or increasing the tumor infiltration of T-cells and/or NK cells in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the doses are administered between about 2 to 5 weeks apart over a duration of between about 1 to 4 months.

494. A method of enhancing, promoting, and/or increasing the tumor infiltration of T-cells and/or NK cells in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein:
a. each dose comprises between about 200 μg to about 30000 μg of the fusion protein; and
b. at least two of the two or more doses are administered at least 2 weeks apart over a duration of at least 1 month.

495. A method of enhancing, promoting, and/or increasing the tumor infiltration of T-cells and/or NK cells in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein:
a. each dose comprises between about 200 μg to about 30000 μg of the fusion protein; and
b. at least two of the two or more doses are administered between about 2 to 5 weeks apart over a duration of between about 1 to 4 months.

496. A method of enhancing, promoting, and/or increasing the tumor infiltration of T-cells and/or NK cells in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein:
a. each dose comprises between about 200 μg to about 30000 μg of the fusion protein; and
b. at least two of the doses are administered between about 2 to 5 weeks apart over a duration of between about 1 to 4 months.

497. A method of enhancing, promoting, and/or increasing the tumor infiltration of T-cells and/or NK cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

498. A method of enhancing, promoting, and/or increasing the tumor infiltration of T-cells and/or NK cells in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

499. A method of enhancing, promoting, and/or increasing the tumor infiltration of T-cells and/or NK cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months.

500. A method of enhancing, promoting, and/or increasing the tumor infiltration of T-cells and/or NK cells in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B.

501. A method of enhancing, promoting, and/or accelerating the recovery from or reversing the effects of lymphopenia in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

502. A method of enhancing, promoting, and/or accelerating the recovery from or reversing the effects of lymphopenia in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

503. A method of enhancing, promoting, and/or accelerating the recovery from or reversing the effects of lymphopenia in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

504. A method of enhancing, promoting, and/or accelerating the recovery from or reversing the effects of lymphopenia in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

505. A method of enhancing, promoting, and/or accelerating the recovery from or reversing the effects of lymphopenia in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

506. A method of enhancing, promoting, and/or accelerating the recovery from or reversing the effects of lymphopenia in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

507. A method of enhancing, promoting, and/or accelerating the recovery from or reversing the effects of lymphopenia in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

508. A method of enhancing, promoting, and/or accelerating the recovery from or reversing the effects of lymphopenia in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

509. A method of enhancing, promoting, and/or accelerating the recovery from or reversing the effects of lymphopenia in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the two or more doses are administered at least two weeks apart,

510. A method of enhancing, promoting, and/or accelerating the recovery from or reversing the effects of lymphopenia in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the two or more doses are administered between 2 to 5 weeks apart.

511. A method of enhancing, promoting, and/or accelerating the recovery from or reversing the effects of lymphopenia in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the two or more doses are administered at least about 2 weeks apart over a duration of at least about 1 month.

512. A method of enhancing, promoting, and/or accelerating the recovery from or reversing the effects of lymphopenia in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the two or more doses are administered between about 2 to 5 weeks apart over a duration of between about 1 to 4 months.

513. A method of enhancing, promoting, and/or accelerating the recovery from or reversing the effects of lymphopenia in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein at least two of the doses are administered between about 2 to 5 weeks apart over a duration of between about 1 to 4 months.

514. A method of enhancing, promoting, and/or accelerating the recovery from or reversing the effects of lymphopenia in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein:
a. each dose comprises between about 200 μg to about 30000 μg of the fusion protein; and
b. at least two of the two or more doses are administered at least 2 weeks apart over a duration of at least 1 month.

515. A method of enhancing, promoting, and/or accelerating the recovery from or reversing the effects of lymphopenia in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein:
a. each dose comprises between about 200 μg to about 30000 μg of the fusion protein; and
b. at least two of the two or more doses are administered between about 2 to 5 weeks apart over a duration of between about 1 to 4 months.

516. A method of enhancing, promoting, and/or accelerating the recovery from or reversing the effects of lymphopenia in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, wherein:
a. each dose comprises between about 200 μg to about 30000 μg of the fusion protein; and
b. at least two of the doses are administered between about 2 to 5 weeks apart over a duration of between about 1 to 4 months.

517. A method of enhancing, promoting, and/or accelerating the recovery from or reversing the effects of lymphopenia in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

518. A method of enhancing, promoting, and/or accelerating the recovery from or reversing the effects of lymphopenia in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

519. A method of enhancing, promoting, and/or accelerating the recovery from or reversing the effects of lymphopenia in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months.

520. A method of enhancing, promoting, and/or accelerating the recovery from or reversing the effects of lymphopenia in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B.

521. A method of enhancing, improving, and/or increasing the response to an anticancer therapy in a subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of an anticancer agent.

522. A method of enhancing, improving, and/or increasing the response to an anticancer therapy in a subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of an anticancer agent.

523. A method of enhancing, improving, and/or increasing the response to an anticancer therapy in a subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of an anticancer agent.

524. A method of enhancing, improving, and/or increasing the response to an anticancer therapy in a subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of an anticancer agent.

525. A method of enhancing, improving, and/or increasing the response to an anticancer therapy in a human subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of an anticancer agent.

526. A method of enhancing, improving, and/or increasing the response to an anticancer therapy in a human subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of an anticancer agent.

527. A method of enhancing, improving, and/or increasing the response to an anticancer therapy in a human subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of an anticancer agent.

528. A method of enhancing, improving, and/or increasing the response to an anticancer therapy in a human subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of an anticancer agent.

529. A method of enhancing, improving, and/or increasing the response to an anticancer therapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered at least two weeks apart, and (II) an effective amount of an anticancer agent.

530. A method of enhancing, improving, and/or increasing the response to an anticancer therapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered between 2 to 5 weeks apart; and (II) an effective amount of an anticancer agent.

531. A method of enhancing, improving, and/or increasing the response to an anticancer therapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered at least about 2 weeks apart over a duration of at least about 1 month; and (II) an effective amount of an anticancer agent.

532. A method of enhancing, improving, and/or increasing the response to an anticancer therapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered between about 2 to 5 weeks apart over a duration of between about 1 to 4 months; and (II) an effective amount of an anticancer agent.

533. A method of enhancing, improving, and/or increasing the response to an anticancer therapy in a subject in need thereof, comprising co-administering to the subject (I) between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the doses are administered between about 2 to 5 weeks apart over a duration of between about 1 to 4 months; and (II) an effective amount of an anticancer agent.

534. A method of enhancing, improving, and/or increasing the response to an anticancer therapy in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of an anticancer agent, wherein:
a. each dose comprises between about 200 μg to about 30000 μg of the fusion protein; and
b. at least two of the two or more doses are administered at least 2 weeks apart over a duration of at least 1 month.

535. A method of enhancing, improving, and/or increasing the response to an anticancer therapy in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of an anticancer agent, wherein:
a. each dose comprises between about 200 μg to about 30000 μg of the fusion protein; and
b. at least two of the two or more doses are administered between about 2 to 5 weeks apart over a duration of between about 1 to 4 months.

536. A method of enhancing, improving, and/or increasing the response to an anticancer therapy in a subject in need thereof, comprising co-administering to the subject (I) between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of an anticancer agent, wherein:
a. each dose comprises between about 200 μg to about 30000 μg of the fusion protein; and
b. at least two of the doses are administered between about 2 to 5 weeks apart over a duration of between about 1 to 4 months.

537. A method of enhancing, improving, and/or increasing the response to an anticancer therapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of an anticancer agent, wherein administering the fusion protein comprises:
A. administering to the subject two or more doses of the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

538. A method of enhancing, improving, and/or increasing the response to an anticancer therapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of an anticancer agent, wherein administering the fusion protein comprises:
A. administering to the subject two or more doses of the fusion protein, wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

539. A method of enhancing, improving, and/or increasing the response to an anticancer therapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of an anticancer agent, wherein administering the fusion protein comprises:
A. administering to the subject two or more doses of an effective amount the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months.

540. A method of enhancing, improving, and/or increasing the response to an anticancer therapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of an anticancer agent, wherein administering the fusion protein comprises:
A. administering to the subject two or more doses of an effective amount the fusion protein, wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B.

541. A method of enhancing, improving, and/or increasing the response to sacituzumab govitecan in a subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

542. A method of enhancing, improving, and/or increasing the response to sacituzumab govitecan in a subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

543. A method of enhancing, improving, and/or increasing the response to sacituzumab govitecan in a subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

544. A method of enhancing, improving, and/or increasing the response to sacituzumab govitecan in a subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

545. A method of enhancing, improving, and/or increasing the response to sacituzumab govitecan in a human subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

546. A method of enhancing, improving, and/or increasing the response to sacituzumab govitecan in a human subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

547. A method of enhancing, improving, and/or increasing the response to sacituzumab govitecan in a human subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

548. A method of enhancing, improving, and/or increasing the response to sacituzumab govitecan in a human subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

549. A method of enhancing, improving, and/or increasing the response to sacituzumab govitecan in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered at least two weeks apart, and (II) an effective amount of sacituzumab govitecan.

550. A method of enhancing, improving, and/or increasing the response to sacituzumab govitecan in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered between 2 to 5 weeks apart; and (II) an effective amount of sacituzumab govitecan.

551. A method of enhancing, improving, and/or increasing the response to sacituzumab govitecan in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered at least about 2 weeks apart over a duration of at least about 1 month; and (II) an effective amount of sacituzumab govitecan.

552. A method of enhancing, improving, and/or increasing the response to sacituzumab govitecan in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered between about 2 to 5 weeks apart over a duration of between about 1 to 4 months; and (II) an effective amount of sacituzumab govitecan.

553. A method of enhancing, improving, and/or increasing the response to sacituzumab govitecan in a subject in need thereof, comprising co-administering to the subject (I) between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the doses are administered between about 2 to 5 weeks apart over a duration of between about 1 to 4 months; and (II) an effective amount of sacituzumab govitecan.

554. A method of enhancing, improving, and/or increasing the response to sacituzumab govitecan in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan, wherein:
a. each dose comprises between about 200 μg to about 30000 μg of the fusion protein; and
b. at least two of the two or more doses are administered at least 2 weeks apart over a duration of at least 1 month.

555. A method of enhancing, improving, and/or increasing the response to sacituzumab govitecan in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan, wherein:
a. each dose comprises between about 200 μg to about 30000 μg of the fusion protein; and
b. at least two of the two or more doses are administered between about 2 to 5 weeks apart over a duration of between about 1 to 4 months.

556. A method of enhancing, improving, and/or increasing the response to sacituzumab govitecan in a subject in need thereof, comprising co-administering to the subject (I) between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan, wherein:
a. each dose comprises between about 200 μg to about 30000 μg of the fusion protein; and
b. at least two of the doses are administered between about 2 to 5 weeks apart over a duration of between about 1 to 4 months.

557. A method of enhancing, improving, and/or increasing the response to sacituzumab govitecan in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan, wherein administering the fusion protein comprises:
A. administering to the subject two or more doses of the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

558. A method of enhancing, improving, and/or increasing the response to sacituzumab govitecan in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan, wherein administering the fusion protein comprises:
A. administering to the subject two or more doses of the fusion protein, wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

559. A method of enhancing, improving, and/or increasing the response to sacituzumab govitecan in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan, wherein administering the fusion protein comprises:
A. administering to the subject two or more doses of an effective amount the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months.

560. A method of enhancing, improving, and/or increasing the response to sacituzumab govitecan in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan, wherein administering the fusion protein comprises:
A. administering to the subject two or more doses of an effective amount the fusion protein, wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B.

561. A method of enhancing, improving, and/or increasing the response to magrolimab in a subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of magrolimab.

562. A method of enhancing, improving, and/or increasing the response to magrolimab in a subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of magrolimab.

563. A method of enhancing, improving, and/or increasing the response to magrolimab in a subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of magrolimab.

564. A method of enhancing, improving, and/or increasing the response to magrolimab in a subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of magrolimab.

565. A method of enhancing, improving, and/or increasing the response to magrolimab in a human subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of magrolimab.

566. A method of enhancing, improving, and/or increasing the response to magrolimab in a human subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of magrolimab.

567. A method of enhancing, improving, and/or increasing the response to magrolimab in a human subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of magrolimab.

568. A method of enhancing, improving, and/or increasing the response to magrolimab in a human subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of magrolimab.

569. A method of enhancing, improving, and/or increasing the response to magrolimab in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered at least two weeks apart, and (II) an effective amount of magrolimab.

570. A method of enhancing, improving, and/or increasing the response to magrolimab in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered between 2 to 5 weeks apart; and (II) an effective amount of magrolimab.

571. A method of enhancing, improving, and/or increasing the response to magrolimab in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered at least about 2 weeks apart over a duration of at least about 1 month; and (II) an effective amount of magrolimab.

572. A method of enhancing, improving, and/or increasing the response to magrolimab in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered between about 2 to 5 weeks apart over a duration of between about 1 to 4 months; and (II) an effective amount of magrolimab.

573. A method of enhancing, improving, and/or increasing the response to magrolimab in a subject in need thereof, comprising co-administering to the subject (I) between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the doses are administered between about 2 to 5 weeks apart over a duration of between about 1 to 4 months; and (II) an effective amount of magrolimab.

574. A method of enhancing, improving, and/or increasing the response to magrolimab in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of magrolimab, wherein:
a. each dose comprises between about 200 μg to about 30000 μg of the fusion protein; and
b. at least two of the two or more doses are administered at least 2 weeks apart over a duration of at least 1 month.

575. A method of enhancing, improving, and/or increasing the response to magrolimab in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of magrolimab, wherein:
a. each dose comprises between about 200 μg to about 30000 μg of the fusion protein; and
b. at least two of the two or more doses are administered between about 2 to 5 weeks apart over a duration of between about 1 to 4 months.

576. A method of enhancing, improving, and/or increasing the response to magrolimab in a subject in need thereof, comprising co-administering to the subject (I) between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of magrolimab, wherein:
a. each dose comprises between about 200 μg to about 30000 μg of the fusion protein; and
b. at least two of the doses are administered between about 2 to 5 weeks apart over a duration of between about 1 to 4 months.

577. A method of enhancing, improving, and/or increasing the response to magrolimab in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of magrolimab, wherein administering the fusion protein comprises:
A. administering to the subject two or more doses of the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

578. A method of enhancing, improving, and/or increasing the response to magrolimab in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of magrolimab, wherein administering the fusion protein comprises:
A. administering to the subject two or more doses of the fusion protein, wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

579. A method of enhancing, improving, and/or increasing the response to magrolimab in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of magrolimab, wherein administering the fusion protein comprises:
A. administering to the subject two or more doses of an effective amount the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months.

580. A method of enhancing, improving, and/or increasing the response to magrolimab in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of magrolimab, wherein administering the fusion protein comprises:
A. administering to the subject two or more doses of an effective amount the fusion protein, wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B.

581. A method of enhancing, improving, and/or increasing the response to an inhibitor of MCL-1 in a subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the MCL-1 inhibitor.

582. A method of enhancing, improving, and/or increasing the response to an inhibitor of MCL-1 in a subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the MCL-1 inhibitor.

583. A method of enhancing, improving, and/or increasing the response to an inhibitor of MCL-1 in a subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the MCL-1 inhibitor.

584. A method of enhancing, improving, and/or increasing the response to an inhibitor of MCL-1 in a subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the MCL-1 inhibitor.

585. A method of enhancing, improving, and/or increasing the response to an inhibitor of MCL-1 in a human subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the MCL-1 inhibitor.

586. A method of enhancing, improving, and/or increasing the response to an inhibitor of MCL-1 in a human subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the MCL-1 inhibitor.

587. A method of enhancing, improving, and/or increasing the response to an inhibitor of MCL-1 in a human subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the MCL-1 inhibitor.

588. A method of enhancing, improving, and/or increasing the response to an inhibitor of MCL-1 in a human subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the MCL-1 inhibitor.

589. A method of enhancing, improving, and/or increasing the response to an inhibitor of MCL-1 in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered at least two weeks apart, and (II) an effective amount of the MCL-1 inhibitor.

590. A method of enhancing, improving, and/or increasing the response to an inhibitor of MCL-1 in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered between 2 to 5 weeks apart; and (II) an effective amount of the MCL-1 inhibitor.

591. A method of enhancing, improving, and/or increasing the response to an inhibitor of MCL-1 in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered at least about 2 weeks apart over a duration of at least about 1 month; and (II) an effective amount of the MCL-1 inhibitor.

592. A method of enhancing, improving, and/or increasing the response to an inhibitor of MCL-1 in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered between about 2 to 5 weeks apart over a duration of between about 1 to 4 months; and (II) an effective amount of the MCL-1 inhibitor.

593. A method of enhancing, improving, and/or increasing the response to an inhibitor of MCL-1 in a subject in need thereof, comprising co-administering to the subject (I) between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the doses are administered between about 2 to 5 weeks apart over a duration of between about 1 to 4 months; and (II) an effective amount of the MCL-1 inhibitor.

594. A method of enhancing, improving, and/or increasing the response to an inhibitor of MCL-1 in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the MCL-1 inhibitor, wherein:
a. each dose comprises between about 200 μg to about 30000 μg of the fusion protein; and
b. at least two of the two or more doses are administered at least 2 weeks apart over a duration of at least 1 month.

595. A method of enhancing, improving, and/or increasing the response to an inhibitor of MCL-1 in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the MCL-1 inhibitor, wherein:
a. each dose comprises between about 200 μg to about 30000 μg of the fusion protein; and
b. at least two of the two or more doses are administered between about 2 to 5 weeks apart over a duration of between about 1 to 4 months.

596. A method of enhancing, improving, and/or increasing the response to an inhibitor of MCL-1 in a subject in need thereof, comprising co-administering to the subject (I) between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the MCL-1 inhibitor, wherein:
a. each dose comprises between about 200 μg to about 30000 μg of the fusion protein; and
b. at least two of the doses are administered between about 2 to 5 weeks apart over a duration of between about 1 to 4 months.

597. A method of enhancing, improving, and/or increasing the response to an inhibitor of MCL-1 in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the MCL-1 inhibitor, wherein administering the fusion protein comprises:
A. administering to the subject two or more doses of the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

598. A method of enhancing, improving, and/or increasing the response to an inhibitor of MCL-1 in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the MCL-1 inhibitor, wherein administering the fusion protein comprises:
A. administering to the subject two or more doses of the fusion protein, wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

599. A method of enhancing, improving, and/or increasing the response to an inhibitor of MCL-1 in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the MCL-1 inhibitor, wherein administering the fusion protein comprises:
A. administering to the subject two or more doses of an effective amount the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months.

600. A method of enhancing, improving, and/or increasing the response to an inhibitor of MCL-1 in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the MCL-1 inhibitor, wherein administering the fusion protein comprises:
A. administering to the subject two or more doses of an effective amount the fusion protein, wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B.

601. A method of promoting, inducing and/or increasing the expansion and/or proliferation of a cell or a population of cells that express fms related tyrosine kinase 3 (FLT3, CD135) in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the expansion and/or proliferation of the cell or population of cells is observed within 5 to 15 days after administration of the fusion protein.

602. A method of promoting, inducing and/or increasing the expansion and/or proliferation of a cell or a population of cells that express fms related tyrosine kinase 3 (FLT3, CD135) in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the expansion and/or proliferation of the cell or population of cells is observed within 5 to 15 days after administration of the fusion protein.

603. A method of promoting, inducing and/or increasing the expansion and/or proliferation of a cell or a population of cells that express fms related tyrosine kinase 3 (FLT3, CD135) in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the expansion and/or proliferation of the cell or population of cells is observed within 5 to 15 days after administration of the fusion protein.

604. A method of promoting, inducing and/or increasing the expansion and/or proliferation of a cell or a population of cells that express fms related tyrosine kinase 3 (FLT3, CD135) in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the expansion and/or proliferation of the cell or population of cells is observed within 5 to 15 days after administration of the fusion protein.

605. A method of promoting, inducing and/or increasing the expansion and/or proliferation of a cell or a population of cells that express fms related tyrosine kinase 3 (FLT3, CD135) in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the expansion and/or proliferation of the cell or population of cells is observed within 5 to 15 days after administration of the fusion protein.

606. A method of promoting, inducing and/or increasing the expansion and/or proliferation of a cell or a population of cells that express fms related tyrosine kinase 3 (FLT3, CD135) in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the expansion and/or proliferation of the cell or population of cells is observed within 5 to 15 days after administration of the fusion protein.

607. A method of promoting, inducing and/or increasing the expansion and/or proliferation of a cell or a population of cells that express fms related tyrosine kinase 3 (FLT3, CD135) in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the expansion and/or proliferation of the cell or population of cells is observed within 5 to 15 days after administration of the fusion protein.

608. A method of promoting, inducing and/or increasing the expansion and/or proliferation of a cell or a population of cells that express fms related tyrosine kinase 3 (FLT3, CD135) in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the expansion and/or proliferation of the cell or population of cells is observed within 5 to 15 days after administration of the fusion protein.

609. A method of promoting, inducing and/or increasing the expansion and/or proliferation of a cell or a population of cells that express fms related tyrosine kinase 3 (FLT3, CD135) in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
a. at least two of the two or more doses are administered at least two weeks apart; and
b. the expansion and/or proliferation of the cell or population of cells is observed within 5 to 15 days after administration of the fusion protein.

610. A method of promoting, inducing and/or increasing the expansion and/or proliferation of a cell or a population of cells that express fms related tyrosine kinase 3 (FLT3, CD135) in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
a. at least two of the two or more doses are administered between 2 to 5 weeks apart; and
b. the expansion and/or proliferation of the cell or population of cells is observed within 5 to 15 days after administration of the fusion protein.

611. A method of promoting, inducing and/or increasing the expansion and/or proliferation of a cell or a population of cells that express fms related tyrosine kinase 3 (FLT3, CD135) in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
a. at least two of the two or more doses are administered at least about 2 weeks apart over a duration of at least about 1 month; and
b. the expansion and/or proliferation of the cell or population of cells is observed within 5 to 15 days after administration of the fusion protein.

612. A method of promoting, inducing and/or increasing the expansion and/or proliferation of a cell or a population of cells that express fms related tyrosine kinase 3 (FLT3, CD135) in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
a. at least two of the two or more doses are administered between about 2 to 5 weeks apart over a duration of between about 1 to 4 months; and
b. the expansion and/or proliferation of the cell or population of cells is observed within 5 to 15 days after administration of the fusion protein.

613. A method of promoting, inducing and/or increasing the expansion and/or proliferation of a cell or a population of cells that express fms related tyrosine kinase 3 (FLT3, CD135) in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
a. at least two of the doses are administered between about 2 to 5 weeks apart over a duration of between about 1 to 4 months; and
b. the expansion and/or proliferation of the cell or population of cells is observed within 5 to 15 days after administration of the fusion protein.

614. A method of promoting, inducing and/or increasing the expansion and/or proliferation of a cell or a population of cells that express fms related tyrosine kinase 3 (FLT3, CD135) in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
a. each dose comprises between about 200 μg to about 30000 μg of the fusion protein;
b. at least two of the two or more doses are administered at least 2 weeks apart over a duration of at least 1 month; and
c. the expansion and/or proliferation of the cell or population of cells is observed within 5 to 15 days after administration of the fusion protein.

615. A method of promoting, inducing and/or increasing the expansion and/or proliferation of a cell or a population of cells that express fms related tyrosine kinase 3 (FLT3, CD135) in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
a. each dose comprises between about 200 μg to about 30000 μg of the fusion protein;
b. at least two of the two or more doses are administered between about 2 to 5 weeks apart over a duration of between about 1 to 4 months; and
c. the expansion and/or proliferation of the cell or population of cells is observed within 5 to 15 days after administration of the fusion protein.

616. A method of promoting, inducing and/or increasing the expansion and/or proliferation of a cell or a population of cells that express fms related tyrosine kinase 3 (FLT3, CD135) in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
a. each dose comprises between about 200 μg to about 30000 μg of the fusion protein;
b. at least two of the doses are administered between about 2 to 5 weeks apart over a duration of between about 1 to 4 months; and
c. the expansion and/or proliferation of the cell or population of cells is observed within 5 to 15 days after administration of the fusion protein.

617. A method of promoting, inducing and/or increasing the expansion and/or proliferation of a cell or a population of cells that express fms related tyrosine kinase 3 (FLT3, CD135) in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
   i. the dosing interval for the two or more doses is one dose every 2 to 4 weeks; and
   ii. the expansion and/or proliferation of the cell or population of cells is observed within 5 to 15 days after administration of the fusion protein, and
B. administering one or more subsequent doses of an effective amount of the fusion protein to the subject, wherein the dosing interval between the last dose of step A and the first dose of step B is greater than the dosing interval for the two or more doses of step A.

618. A method of promoting, inducing and/or increasing the expansion and/or proliferation of a cell or a population of cells that express fms related tyrosine kinase 3 (FLT3, CD135) in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
   i. each dose comprises between about 200 μg to about 30000 μg of the fusion protein;
   ii. the dosing interval for the two or more doses is once every 2 to 4 weeks; and
   iii. the expansion and/or proliferation of the cell or population of cells is observed within 5 to 15 days after administration of the fusion protein, and
B. administering one or more subsequent doses of an effective amount of the fusion protein to the subject, wherein the dosing interval between the last dose of step A and the first dose of step B is greater than the dosing interval for the two or more doses of step A.

619. A method of promoting, inducing and/or increasing the expansion and/or proliferation of a cell or a population of cells that express fms related tyrosine kinase 3 (FLT3, CD135) in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
   i. the dosing interval for the two or more doses is one dose every 2 to 4 weeks; and
   ii. the expansion and/or proliferation of the cell or population of cells is observed within 5 to 15 days after administration of the fusion protein; and
B. administering one or more subsequent doses of an effective amount of the fusion protein to the subject, wherein the dosing interval between the last dose of step A and the first dose of step B is between about 6 weeks to about 8 months.

620. A method of promoting, inducing and/or increasing the expansion and/or proliferation of a cell or a population of cells that express fms related tyrosine kinase 3 (FLT3, CD135) in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
   i. the two or more doses are administered about 8 to 20 days apart; and
   ii. the expansion and/or proliferation of the cell or population of cells is observed within 5 to 15 days after administration of the fusion protein,
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B.

621. A method of enhancing, improving, and/or increasing the response to an immunotherapy in a subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the immunotherapy.

622. A method of enhancing, improving, and/or increasing the response to an immunotherapy in a subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the immunotherapy.

623. A method of enhancing, improving, and/or increasing the response to an immunotherapy in a subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the immunotherapy.

624. A method of enhancing, improving, and/or increasing the response to an immunotherapy in a subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the immunotherapy.

625. A method of enhancing, improving, and/or increasing the response to an immunotherapy in a human subject in need thereof, comprising co-administering to the subject (I) at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the immunotherapy.

626. A method of enhancing, improving, and/or increasing the response to an immunotherapy in a human subject in need thereof, comprising co-administering to the subject (I) at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the immunotherapy.

627. A method of enhancing, improving, and/or increasing the response to an immunotherapy in a human subject in need thereof, comprising co-administering to the subject (I) at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the immunotherapy.

628. A method of enhancing, improving, and/or increasing the response to an immunotherapy in a human subject in need thereof, comprising co-administering to the subject (I) between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the immunotherapy.

629. A method of enhancing, improving, and/or increasing the response to an immunotherapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered at least two weeks apart, and (II) an effective amount of the immunotherapy.

630. A method of enhancing, improving, and/or increasing the response to an immunotherapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered between 2 to 5 weeks apart; and (II) an effective amount of the immunotherapy.

631. A method of enhancing, improving, and/or increasing the response to an immunotherapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered at least about 2 weeks apart over a duration of at least about 1 month; and (II) an effective amount of the immunotherapy.

632. A method of enhancing, improving, and/or increasing the response to an immunotherapy in a subject in need thereof, comprising co-administering to the subject (I) two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered between about 2 to 5 weeks apart over a duration of between about 1 to 4 months; and (II) an effective amount of the immunotherapy.

633. A method of enhancing, improving, and/or increasing the response to an immunotherapy in a subject in need thereof, comprising co-administering to the subject (I) between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the doses are administered between about 2 to 5 weeks apart over a duration of between about 1 to 4 months; and (II) an effective amount of the immunotherapy.

634. A method of enhancing, improving, and/or increasing the response to an immunotherapy in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the immunotherapy, wherein:
a. each dose comprises between about 200 μg to about 30000 μg of the fusion protein; and
b. at least two of the two or more doses are administered at least 2 weeks apart over a duration of at least 1 month.

635. A method of enhancing, improving, and/or increasing the response to an immunotherapy in a subject in need thereof, comprising co-administering to the subject (I) two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the immunotherapy, wherein:
a. each dose comprises between about 200 μg to about 30000 μg of the fusion protein; and
b. at least two of the two or more doses are administered between about 2 to 5 weeks apart over a duration of between about 1 to 4 months.

636. A method of enhancing, improving, and/or increasing the response to an immunotherapy in a subject in need thereof, comprising co-administering to the subject (I) between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the immunotherapy, wherein:
a. each dose comprises between about 200 μg to about 30000 μg of the fusion protein; and
b. at least two of the doses are administered between about 2 to 5 weeks apart over a duration of between about 1 to 4 months.

637. A method of enhancing, improving, and/or increasing the response to an immunotherapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the immunotherapy, wherein administering the fusion protein comprises:
A. administering to the subject two or more doses of the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

638. A method of enhancing, improving, and/or increasing the response to an immunotherapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the immunotherapy, wherein administering the fusion protein comprises:
A. administering to the subject two or more doses of the fusion protein, wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

639. A method of enhancing, improving, and/or increasing the response to an immunotherapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the immunotherapy, wherein administering the fusion protein comprises:
A. administering to the subject two or more doses of an effective amount the fusion protein, wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months.

640. A method of enhancing, improving, and/or increasing the response to an immunotherapy in a subject in need thereof, comprising co-administering to the subject (I) a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14, and (II) an effective amount of the immunotherapy, wherein administering the fusion protein comprises:
A. administering to the subject two or more doses of an effective amount the fusion protein, wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B.

641. A method of inducing the immune system in a subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

642. A method of inducing the immune system in a subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

643. A method of inducing the immune system in a subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

644. A method of inducing the immune system in a subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

645. A method of inducing the immune system in a human subject in need thereof, comprising administering to the subject at least about 200 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
a. at least 5 amino acids are truncated from the C-terminus of the FLT3L extracellular domain; and/or
b. the Fc region does not comprise a hinge region.

646. A method of inducing the immune system in a human subject in need thereof, comprising administering to the subject at least about 225 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

647. A method of inducing the immune system in a human subject in need thereof, comprising administering to the subject at least about 675 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

648. A method of inducing the immune system in a human subject in need thereof, comprising administering to the subject between about 200 μg to about 30000 μg of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14.

649. A method of inducing the immune system in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered at least two weeks apart.

650. A method of inducing the immune system in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered between 2 to 5 weeks apart.

651. A method of inducing the immune system in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered at least about 2 weeks apart over a duration of at least about 1 month.

652. A method of inducing the immune system in a subject in need thereof, comprising administering to the subject two or more doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the two or more doses are administered between about 2 to 5 weeks apart over a duration of between about 1 to 4 months.

653. A method of inducing the immune system in a subject in need thereof, comprising administering to the subject between about 3 to 8 doses of an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein at least two of the doses are administered between about 2 to 5 weeks apart over a duration of between about 1 to 4 months.

654. A method of inducing the immune system in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
a. each dose comprises between about 200 μg to about 30000 μg of the fusion protein; and
b. at least two of the two or more doses are administered at least 2 weeks apart over a duration of at least 1 month.

655. A method of inducing the immune system in a subject in need thereof, comprising administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
a. each dose comprises between about 200 μg to about 30000 μg of the fusion protein; and
b. at least two of the two or more doses are administered between about 2 to 5 weeks apart over a duration of between about 1 to 4 months.

656. A method of inducing the immune system in a subject in need thereof, comprising administering to the subject between about 3 to 8 doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
a. each dose comprises between about 200 μg to about 30000 μg of the fusion protein; and
b. at least two of the doses are administered between about 2 to 5 weeks apart over a duration of between about 1 to 4 months.

657. A method of inducing the immune system in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

658. A method of inducing the immune system in a subject in need thereof, comprising:
A. administering to the subject two or more doses of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein:
   i. each dose comprises between about 200 μg to about 30000 μg of the fusion protein; and
   ii. the dosing interval for the two or more doses is once every 2 to 4 weeks; and
B. administering one or more subsequent doses of an effective amount of the fusion protein to the subject, wherein the dosing interval between the last dose of step A and the first dose of step B is greater than the dosing interval for the two or more doses of step A.

659. A method of inducing the immune system in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the dosing interval for the two or more doses is one dose every 2 to 4 weeks; and
B. administering one or more subsequent doses of an effective amount of the fusion protein to the subject, wherein the dosing interval between the last dose of step A and the first dose of step B is between about 6 weeks to about 8 months.

660. A method of inducing the immune system in a subject in need thereof, comprising:
A. administering to the subject two or more doses of an effective amount a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and wherein the two or more doses are administered about 8 to 20 days apart;
B. administering to the subject two or more subsequent doses of an effective amount of the fusion protein, wherein the dosing interval for the two or more subsequent doses is between about 21 to 36 days apart;
C. pausing administration of the fusion protein to the subject for a period of between about 6 weeks to about 8 months; and
D. repeating the administration of any one of steps A and B.

661. A method of preventing, reducing and/or inhibiting the recurrence, growth, proliferation, migration and/or metastasis of a cancer cell or population of cancer cells in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

662. A method of treating and/or inhibiting cancer in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

663. A method of enhancing, promoting, and/or increasing the tumor infiltration of T-cells and/or NK cells in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

664. A method of enhancing, promoting, and/or accelerating the recovery from or reversing the effects of lymphopenia in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

665. A method of promoting, inducing and/or increasing the expansion and/or proliferation of a cell or a population of cells that express fms related tyrosine kinase 3 (FLT3, CD135) in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

666. A method of inducing the immune system in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of sacituzumab govitecan.

667. A method of preventing, reducing and/or inhibiting the recurrence, growth, proliferation, migration and/or metastasis of a cancer cell or population of cancer cells in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor.

668. A method of treating and/or inhibiting cancer in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor.

669. A method of enhancing, promoting, and/or increasing the tumor infiltration of T-cells and/or NK cells in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor.

670. A method of enhancing, promoting, and/or accelerating the recovery from or reversing the effects of lymphopenia in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor.

671. A method of promoting, inducing and/or increasing the expansion and/or proliferation of a cell or a population of cells that express fms related tyrosine kinase 3 (FLT3, CD135) in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor.

672. A method of inducing the immune system in a subject in need thereof, comprising administering to the subject (I) an effective amount of a fusion protein comprising a human fms related tyrosine kinase 3 ligand (FLT3L) extracellular domain operably linked to an immunoglobulin fragment crystallizable region (Fc region), wherein the fusion protein comprises the amino acid sequence of SEQ ID NO: 14; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor.

673. A method of preventing, reducing and/or inhibiting the recurrence, growth, proliferation, migration and/or metastasis of a cancer cell or population of cancer cells in a subject in need thereof, comprising administering to the subject (I) an effective amount of a human fms related tyrosine kinase 3 ligand (FLT3L) modulator; and (II) an effective amount of sacituzumab govitecan.

674. A method of treating and/or inhibiting cancer in a subject in need thereof, comprising administering to the subject (I) an effective amount of a human fms related tyrosine kinase 3 ligand (FLT3L) modulator; and (II) an effective amount of sacituzumab govitecan.

675. A method of enhancing, promoting, and/or increasing the tumor infiltration of T-cells and/or NK cells in a subject in need thereof, comprising administering to the subject (I) an effective amount of a human fms related tyrosine kinase 3 ligand (FLT3L) modulator; and (II) an effective amount of sacituzumab govitecan.

676. A method of enhancing, promoting, and/or accelerating the recovery from or reversing the effects of lymphopenia in a subject in need thereof, comprising administering to the subject (I) an effective amount of a human fms related tyrosine kinase 3 ligand (FLT3L) modulator; and (II) an effective amount of sacituzumab govitecan.

677. A method of promoting, inducing and/or increasing the expansion and/or proliferation of a cell or a population of cells that express fms related tyrosine kinase 3 (FLT3, CD135) in a subject in need thereof, comprising administering to the subject (I) an effective amount of a human fms related tyrosine kinase 3 ligand (FLT3L) modulator; and (II) an effective amount of sacituzumab govitecan.

678. A method of inducing the immune system in a subject in need thereof, comprising administering to the subject (I) an effective amount of a human fms related tyrosine kinase 3 ligand (FLT3L) modulator; and (II) an effective amount of sacituzumab govitecan.

679. A method of preventing, reducing and/or inhibiting the recurrence, growth, proliferation, migration and/or metastasis of a cancer cell or population of cancer cells in a subject in need thereof, comprising administering to the subject (I) an effective amount human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor.

680. A method of treating and/or inhibiting cancer in a subject in need thereof, comprising administering to the subject (I) an effective amount of a human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor.

681. A method of enhancing, promoting, and/or increasing the tumor infiltration of T-cells and/or NK cells in a subject in need thereof, comprising administering to the subject (I) an effective amount of a fusion protein comprising a human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor.

682. A method of enhancing, promoting, and/or accelerating the recovery from or reversing the effects of lymphopenia in a subject in need thereof, comprising administering to the subject (I) an effective amount of a human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor.

683. A method of promoting, inducing and/or increasing the expansion and/or proliferation of a cell or a population of cells that express fms related tyrosine kinase 3 (FLT3, CD135) in a subject in need thereof, comprising administering to the subject (I) an effective amount of a human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor.

684. A method of inducing the immune system in a subject in need thereof, comprising administering to the subject (I) an effective amount of a human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor.

685. The method of any one of embodiments 673-684, wherein the FLT3L modulator is a fusion protein comprising a FLT3L protein or fragment thereof and an Fc protein or fragment thereof.

686. The method of embodiment 685, wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18, 21-27, 114, and 115.

687. The method of embodiment 685, wherein the Fc protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 111.

688. The method of embodiment 685 or 687, wherein residues 13-17 of SEQ ID NO: 111 comprise the amino acid sequence PVAGT (SEQ ID NO: 116) and residue 76 of SEQ ID NO: 111 is a glycine.

689. The method of embodiment 685, 687, or 688, wherein the FLT3L protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NOs: 112, 113, or 117.

690. The method of embodiment 685, 687, or 688, wherein the FLT3L protein or fragment thereof comprises CDX-301.

691. The method of any one of embodiments 667-672, wherein the immunoconjugate is co-administered with the fusion protein.

692. The method of any one of embodiments 679-691, the immunoconjugate is co-administered with the FLT3L modulator.

693. The method of any one of embodiments 679-692, wherein the FLT3L modulator comprises the amino acid sequence of any one of SEQ ID NOs: 101-105 and 107.

694. The method of any one of embodiments 679-693, wherein the immunoconjugate comprises datopotamab deruxtecan (DS-1062).

695. The method of embodiment 691, 692, or 693, wherein the immunconjugate comprises an anti-Trop2-ADC.

696. The method of claim 695, wherein the anti-Trop-2 ADC comprises a topoisomerase I inhibitor.

697. The method of claim 696, wherein the topoisomerase I inhibitor is selected from irinotecan, topetecan and SN-38.

698. The method of any one of claims 695-697, wherein the anti-Trop-2 ADC has a structural formula of mAb-CL2A-SN-38, with a structure represented by:

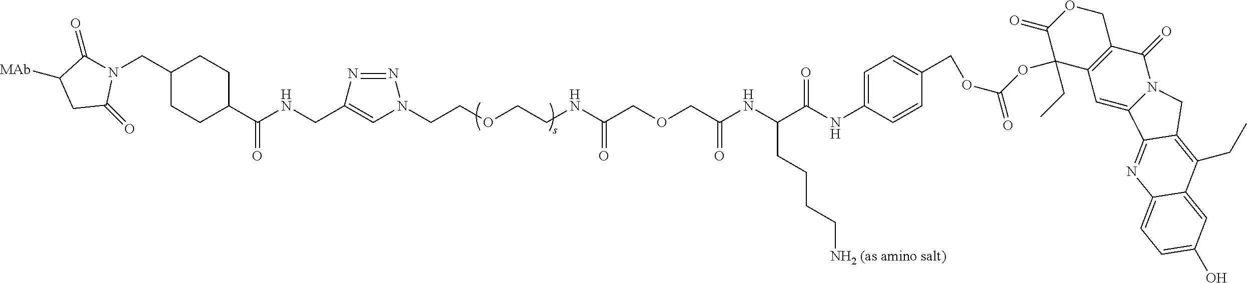

(described, e.g., in U.S. Pat. No. 7,999,083).

699. The method of any one of claims 695-697, wherein the anti-Trop-2 ADC comprises sacituzumab (hRS7; described, e.g., in WO2003074566, FIGS. 3 and 4).

700. The method of any one of claims 695-697, wherein the anti-Trop-2 ADC is selected from sacituzumab govitecan, datopotamab deruxtecan (DS-1062), ESG-401, SKB-264, DAC-02 and BAT-8003.

701. The method of any one of claims 695-697, wherein the anti-Trop-2 ADC comprises sacituzumab govitecan.

702. The method of embodiment 691, wherein the immunoconjugate comprises an anti-Trop2 antibody.

703. The method of any one of embodiments 667-672, wherein the FLT3R agonist is co-administered with the fusion protein.

704. The method of embodiment 703, wherein the FLT3R agonist is selected from an antibody, small molecule, or cytokine.

705. The method of any one of embodiments 667-672, wherein the anti-PD1 antibody is co-administered with the fusion protein.

706. The method of embodiment 705, wherein the anti-PD1 antibody is selected from balstilimab, budigalimab, camrelizumab, cemiplimab, cetrelimab, dostarlimab, genolimzumab, nivolumab, pembrolizumab, pidilizumab, prolgolimab, retifanlimab, sasanlimab, sintilimab, spartalizumab, tislelizumab, toripalimab, and zimberelimab.

707. The method of any one of embodiments 667-672, wherein the anti-Tigit antibody is co-administered with the fusion protein.

708. The method of embodiment 707, wherein the anti-Tigit antibody is AB-308, AGEN-1307 (AGEN-1327), AGEN-1777, AK127, BMS-986207, domvanalimab, EOS-448, etigilimab, JS006, ociperlimab, SEA-TGT (SGN-TGT), tiragolumab, and vibostolimab.

709. The method of any one of embodiments 667-672, wherein the CD73 inhibitor is co-administered with the fusion protein.

710. The method of embodiment 709, wherein the CD73 inhibitor is a small molecule.

711. The method of embodiment 709 or 710, wherein the CD73 inhibitor is AB680 (quemliclustat).

712. The method of any one of embodiments 667-672, wherein the adenosine receptor antagonist is co-administered with the fusion protein.

713. The method of embodiment 712, wherein the adenosine receptor antagonist is a small molecule.

714. The method of embodiment 712 or 713, wherein the adenosine receptor antagonist is AB729 (etrumadenant).

715. The method of any one of embodiments 667-672, wherein the anti-CCR8 antibody is co-administered with the fusion protein.

716. The method of embodiment 715, wherein the anti-CCR8 antibody is JTX-1811.

717. The method of any one of embodiments 161-180, 381-400, 601-620, 665, 671, and 691-716, wherein the cell or population of cells that express FLT3 comprise dendritic cells (e.g., cDC1 cells and/or cDC2 cells), monocyte-derived dendritic cells (moDCs), and/or progenitor cells thereof.

718. The method of any one of embodiments 161-180, 381-400, 601-620, 665, 671, and 691-716, wherein the cell or population of cells that express FLT3 comprise hematopoietic progenitor cells.

719. The method of embodiment 718, wherein the hematopoietic progenitor cells are selected from the group consisting of: Common Lymphoid Progenitors (CLPs), Early Progenitors with Lymphoid and Myeloid potential (EPLMs,), granulocyte-monocyte (GM) progenitors (GMP), monocyte-derived dendritic cells (moDCs) progenitors, and early multi-potent progenitors (MPP) within the Lineage⁻kit⁺Sca1− (LSK) compartment.

720. The method of any one of embodiments 161-180, 381-400, 601-620, 665, 671, and 691-719, wherein the cell or population of cells are expanded within a solid tumor.

721. The method of any one of embodiments 161-180, 381-400, 601-620, 665, 671, and 691-720, wherein conventional dendritic cells (e.g., cDC1 and/or cDC2) are expanded or induced to proliferate.

722. The method of embodiment 721, wherein cDC1 dendritic cells (e.g., positive for surface expression of X-C motif chemokine receptor 1 (XCR1), thrombomodulin (THBD, CD141), and C-type lectin domain containing 9A (CLEC9A)) are expanded or induced to proliferate.

723. The method of embodiment 722, wherein cDC2 dendritic cells (e.g., positive for surface expression of CD1c molecule (BDCA) are expanded or induced to proliferate.

724. The method of any one of embodiments 161-180, 381-400, 601-620, 665, 671, and 691-723, wherein peak expansion of the cell or population of cells is observed within 7 to 14 days after administration of the fusion protein.

725. The method of any one of embodiments 161-180, 381-400, 601-620, 665, 671, and 691-723, wherein peak expansion of the cell or population of cells is observed within 7 to 10 days after administration of the fusion protein.

726. The method of any one of embodiments 161-180, 381-400, 601-620, 665, 671, and 691-723, wherein peak expansion of the cell or population of cells is observed within 8 to 14 days after administration of the fusion protein.

727. The method of any one of embodiments 161-180, 381-400, 601-620, 665, 671, and 691-723, wherein peak expansion of the cell or population of cells is observed within 8 to 10 days after administration of the fusion protein.

728. The method of any one of embodiments 161-180, 381-400, 601-620, 665, 671, and 691-723, wherein peak expansion of the cell or population of cells is observed within about 5, 6, 7, 8, 9, 10, 11, 12, 13, 14, or 15 days after administration of the fusion protein.

729. The method of any preceding embodiment, wherein administering the fusion protein comprises administering a polynucleotide encoding the fusion protein.

730. The method of embodiment 729, wherein the polynucleotide is selected from the group consisting of DNA, cDNA, RNA or mRNA.

731. The method of embodiment 729 or 730, wherein the polynucleotide comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from the group consisting of SEQ ID NOs: 28-70.

732. The method of embodiment 729 or 730, wherein the polynucleotide comprises a nucleic acid selected from the group consisting of SEQ ID NOs: 28-70.

733. The method of any one of embodiments 729-732, wherein the polynucleotide is delivered via a vector.

734. The method of embodiment 733, wherein the vector is a plasmid vector or a viral vector.

735. The method of embodiment 734, wherein the viral vector comprises an oncolytic viral vector.

736. The method of embodiment 734 or 735, wherein the viral vector comprises a DNA virus or a RNA virus.

737. The method of any one of embodiments 734-736, wherein the viral vector is from a viral family selected from the group consisting of: Adenoviridae (e.g., Adenovirus), Arenaviridae (e.g., lymphocytic choriomeningitis mammarenavirus, Cali mammarenavirus (a.k.a., Pichinde mammarenavirus), Poxviridae (e.g., Vaccinia virus), Herpesviridae (e.g., Herpesvirus, e.g., HSV-1), Parvoviridae (e.g., Parvovirus H1), Reoviridae (e.g., Reovirus), Picornaviridae (e.g., Coxsackievirus, Seneca Valley Virus, Poliovirus), Paramyxoviridae (e.g., Measles virus, Newcastle disease virus (NDV)), Rhabdoviridae (e.g., Vesicular stomatitis virus (VSV)), Togaviridae (e.g., Alphavirus, Sindbis virus), Enteroviridae (e.g., Echovirus).

738. The method of any one of embodiments 1-737, wherein the fusion protein is formulated for delivery via a lipid nanoparticle, micelle, liposome, or capsule.

739. The method of embodiment 738, wherein the fusion protein is formulated for delivery via a lipid nanoparticle.

740. The method of any one of embodiments 1-80, 101-300, 321-520, and 541-739, further comprising co-administering to the subject an anticancer agent.

741. The method of any one of embodiments 81-100, 301-320, 521-540, and 740, wherein the anticancer agent is an anti-neoplastic or chemotherapeutic agent.

742. The method of embodiment 741, wherein the anti-neoplastic or chemotherapeutic agent is selected from the group consisting of a nucleoside analog (e.g., 5-fluorouracil, gemcitabine, cytarabine, cladribine, pentostatin, fludarabine), a taxane (e.g., paclitaxel, nab-paclitaxel, docetaxel, cabazitaxel), a platinum coordination complex (cisplatin, carboplatin, oxaliplatin, nedaplatin, triplatin tetranitrate, phenanthriplatin, picoplatin, satraplatin, dicycloplatin, eptaplatin, lobaplatin, miriplatin), a dihydrofolate reductase (DHFR) inhibitor (e.g., methotrexate, trimetrexate, pemetrexed), a topoisomerase inhibitor (e.g., doxorubicin, daunorubicin, dactinomycin, eniposide, epirubicin, etoposide, idarubicin, irinotecan, mitoxantrone, pixantrone, sobuzoxane, topotecan, irinotecan, MM-398 (liposomal irinotecan), vosaroxin and GPX-150, aldoxorubicin, AR-67, mavelertinib, AST-2818, avitinib (ACEA-0010), irofulven (MGI-114)), an alkylating agent (e.g., a nitrogen mustard (e.g., cyclophosphamide, chlormethine, uramustine or uracil mustard, melphalan, chlorambucil, ifosfamide, bendamustine, temozolomide, carmustine), a nitrosourea (e.g., carmustine, lomustine, streptozocin), an alkyl sulfonate (e.g., busulfan)), and mixtures thereof.

743. The method of any one of embodiments 81-100, 301-320, 521-540, and 740, wherein the anticancer agent is an agonist or activator of a toll-like receptor (TLR) or a stimulator of interferon genes (STING) receptor.

744. The method of embodiment 743, wherein the TLR agonist or activator is selected from the group consisting of a TLR2 agonist, a TLR3 agonist, a TLR4 agonist, a TLR5 agonist, a TLR7 agonist, a TLR8 agonist and a TLR9 agonist.

745. The method of embodiment 744, wherein the TLR7 agonist is selected from the group consisting of GS-9620 (vesatolimod), DS-0509, LHC-165 and TMX-101 (imiquimod), and/or wherein the TLR8 agonist is selected from the group consisting of GS-9688 and NKTR-262 (dual TLR7/TLR8 agonist).

746. The method of embodiment 743, wherein the STING receptor agonist or activator is selected from the group consisting of ADU-S100 (MIW-815), SB-11285, MK-1454, SR-8291, AdVCA0848, GSK-532, SYN-STING, MSA-1, SR-8291, 5,6-dimethylxanthenone-4-acetic acid (DMXAA), cyclic-GAMP (cGAMP) and cyclic-di-AMP.

747. The method of embodiment 740, wherein the anticancer agent is an immune checkpoint inhibitor.

748. The method of any one of embodiments 1-180, 201-400, 421-620, and 641-743, further comprising co-administering to the subject an immunotherapy.

749. The method of any one of embodiments 181-200, 401-421, 621-640, and 748, wherein the immunotherapy comprises co-administering one or more antibodies or antigen-binding antibody fragments thereof, or antibody-drug conjugates thereof, CD3-targeting multi-specific molecules, NK cell-activating receptor-targeting multi-specific molecules, or non-immunoglobulin antigen-binding domains or antibody mimetic proteins directed against one or more targets or tumor associated antigens (TAAs) selected from the group consisting of: CD19, MS4A1 (CD20), CD22, IL2RA (CD25), CD27, TNFRSF8 (CD30), CD33, CD37, CD38, CD40, CD44, CD48, CD52, CD70, NT5E (CD73), ENTPD1 (CD39), CD74, CD79b, CD80, CD86, IL3RA (CD123), PROM1 (CD133), CD137, SDC1 (CD138), alpha fetoprotein (AFP), c-Met; c-Kit; C-type lectin domain family 12 member A (CLEC12A, CLL1, CD371); C-type lectin domain containing 9A (CLEC9A, CD370); cadherin 3 (CDH3, p-cadherin, PCAD); carbonic anhydrase 6 (CA6); carbonic anhydrase 9 (CA9, CAIX); carcinoembryonic antigen related cell adhesion molecule 3 (CEACAM3); carcinoembryonic antigen related cell adhesion molecule 5 (CEACAM5); carcinoembryonic antigen related cell adhesion molecule 6 (CEACAM6, CD66c); chorionic somatomammotropin hormone 1 (CSH1, CS1); coagulation factor III, tissue factor (F3, TF); collectin subfamily member 10 (COLEC10); delta like canonical Notch ligand 3 (DLL3); ectonucleotide pyrophosphatase/phosphodiesterase 3 (ENPP3); ephrin A1 (EFNA1); epidermal growth factor receptor (EGFR); EGFR variant III (EGFRvIII); EPH receptor A2 (EPHA2); epithelial cell adhesion molecule (EPCAM); erb-b2 receptor tyrosine kinase 2 (ERBB2; HER2); fibroblast activation protein alpha (FAP); fibroblast growth factor receptor 2 (FGFR2); fibroblast growth factor receptor 3 (FGFR3); folate hydrolase 1 (FOLH1, PSMA); folate receptor 1 (FOLR1, FRa); GD2 ganglioside; glycoprotein NMB (GPNMB, osteoactivin); guanylate cyclase 2C (GUCY2C, GCC); human papillomavirus (HPV) E6; HPV E7; major histocompatibility complex (MHC) class I-presented neoantigens, major histocompatibility complex (MHC) class II-presented neoantigens, major histocompatibility complex, class I, E (HLA-E); major histocompatibility complex, class I, F (HLA-F); major histocompatibility complex, class I, G (HLA-G, MHC-G); integrin subunit beta 7 (ITGB7); leukocyte immunoglobulin like receptor B1 (LILRB1, ILT2); leukocyte immunoglobulin like receptor B2 (LILRB2, ILT4); LY6/PLAUR domain containing 3 (LYPD3, C4.4A); glypican 3 (GPC3); KRAS proto-oncogene, GTPase (KRAS); MAGE family member A1 (MAGEA1); MAGE family member A3 (MAGEA3); MAGE family member A4 (MAGEA4); MAGE family member A11 (MAGEA11); MAGE family member C1 (MAGEC1); MAGE family member C2 (MAGEC2); MAGE family member D1 (MAGED1); MAGE family member D2 (MAGED2); mesothelin (MSLN); mucin 1 (MUC1) and splice variants thereof (e.g., MUC1/C, D, and Z); mucin 16 (MUC16); necdin (NDN); nectin cell adhesion molecule 4 (NECTIN4); SLIT and NTRK like family member 6 (SLITRK6); promyelocytic leukemia (PML, TRIM19); protein tyrosine kinase 7 (inactive) (PTK7); SLAM family member 6 (SLAMF6, CD352); SLAM family member 7 (SLAMF7, 19A, CD319, CRACC, CS1); sialic acid binding Ig like lectin 7 (SIGLEC7); sialic acid binding Ig like lectin 9 (SIGLEC9); solute carrier family 34 (sodium phosphate), member 2 (SLC34A2); solute carrier family 39 member 6 (SLC39A6; LIV1); STEAP family member 1 (STEAP1); TNF receptor superfamily member 4 (TNFRSF4, OX40 or CD134); TNF superfamily member 9 (TNFSF9; 4-1BB-L, CD137L); TNF receptor superfamily member 10a (TNFRSF10A, DR4, CD261, TRAILR1); TNF receptor superfamily member 10b (TNFRSF10B, DR5, CD262, TRAILR2); TNF receptor superfamily member 13B (TNFRSF13B; CD267, TACI, IGAD2); TNF receptor superfamily member 17 (TNFRSF17, BCMA, CD269); TNF receptor superfamily member 18 (TNFRSF18, GITR or CD357); transferrin (TF); transforming growth factor beta 1 (TGFB1); trophoblast glycoprotein (TPBG, 5T4); trophinin (TRO, MAGED3); tumor associated calcium signal transducer 2 (TACSTD2, TROP2, EGP1); Fucosyl GM1; sialyl Lewis adhesion molecule (sLe); and Lewis Y antigen.

750. The method of embodiment 749, wherein the one or more antibodies or antigen-binding antibody fragments thereof, or antibody-drug conjugates thereof, CD3-targeting multi-specific molecules, NK cell-activating receptor-targeting multi-specific molecules, or non-immunoglobulin antigen-binding domains or antibody mimetic proteins binds to an epitope of a target or tumor associated antigen (TAA) presented in a major histocompatibility complex (MHC) molecule.

751. The method of embodiment 750, wherein the NK cell-activating receptor is selected from the group consisting of CD16, NKp30, NKp44, NKp46, NKp80 and NKG2D.

752. The method of any one of embodiments 181-200, 401-420, 621-640, and 748, wherein the immunotherapy comprises co-administering one or more cellular therapies selected from the group consisting of: natural killer (NK) cells, NK-T cells, T cells, cytokine-induced killer (CIK) cells, macrophage (MAC) cells, tumor infiltrating lymphocytes (TILs) and dendritic cells (DCs).

753. The method of embodiment 752, wherein the one or more cellular therapies comprise a T cell therapy selected from the group consisting of: alpha/beta TCR T cells, gamma/delta TCR T cells, regulatory T (Treg) cells and TRuC™ T cells.

754. The method of embodiment 752, wherein the one or more cellular therapies comprise a NK cell therapy comprising NK-92 cells.

755. The method of any one of embodiments 752-754, wherein the one or more cellular therapies comprise cells that are autologous, syngeneic or allogeneic to the subject.

756. The method of any one of embodiments 752-755, wherein the one or more cellular therapies comprise cells comprising chimeric antigen receptors (CARs).

757. The method of any one of embodiments 752-756, wherein the cells in the cellular therapy bind to a target or tumor associated antigen (TAA) selected from the group consisting of selected from the group consisting of: CD19, MS4A1 (CD20), CD22, IL2RA (CD25), CD27, TNFRSF8 (CD30), CD33, CD37, CD38, CD40, CD44, CD48, CD52, CD70, NTSE (CD73), ENTPD1 (CD39), CD74, CD79b, CD80, CD86, IL3RA (CD123), PROM1 (CD133), CD137, SDC1 (CD138), alpha fetoprotein (AFP), c-Met; c-Kit; C-type lectin domain family 12 member A (CLEC12A, CLL1, CD371); C-type lectin domain containing 9A (CLEC9A, CD370); cadherin 3 (CDH3, p-cadherin, PCAD); carbonic anhydrase 6 (CA6); carbonic anhydrase 9 (CA9, CAIX); carcinoembryonic antigen related cell adhesion molecule 3 (CEACAM3); carcinoembryonic antigen related cell adhesion molecule 5 (CEACAM5); carcinoembryonic antigen related cell adhesion molecule 6 (CEACAM6, CD66c); chorionic somatomammotropin hormone 1 (CSH1, CS1); coagulation factor III, tissue factor (F3, TF); collectin subfamily member 10 (COLEC10); delta like canonical Notch ligand 3 (DLL3); ectonucleotide pyrophosphatase/phosphodiesterase 3 (ENPP3); ephrin A1 (EFNA1); epidermal growth factor receptor (EGFR); EGFR variant III (EGFRvIII); EPH receptor A2 (EPHA2); epithelial cell adhesion molecule (EPCAM); erb-b2 receptor tyrosine kinase 2 (ERBB2; HER2); fibroblast activation protein alpha (FAP); fibroblast growth factor receptor 2 (FGFR2); fibroblast growth factor receptor 3 (FGFR3); folate hydrolase 1 (FOLH1, PSMA); folate receptor 1 (FOLR1, FRα); GD2 ganglioside; glycoprotein NMB (GPNMB, osteoactivin); guanylate cyclase 2C (GUCY2C, GCC); human papillomavirus (HPV) E6; HPV E7; major histocompatibility complex (MHC) class I-presented neoantigens, major histocompatibility complex (MHC) class II-presented neoantigens, major histocompatibility complex, class I, E (HLA-E); major histocompatibility complex, class I, F (HLA-F); major histocompatibility complex, class I, G (HLA-G, MHC-G); integrin subunit beta 7 (ITGB7); leukocyte immunoglobulin like receptor B1 (LILRB1, ILT2); leukocyte immunoglobulin like receptor B2 (LILRB2, ILT4); LY6/PLAUR domain containing 3 (LYPD3, C4.4A); glypican 3 (GPC3); KRAS proto-oncogene, GTPase (KRAS); MAGE family member A1 (MAGEA1); MAGE family member A3 (MAGEA3); MAGE family member A4 (MAGEA4); MAGE family member A11 (MAGEA11); MAGE family member C1 (MAGEC1); MAGE family member C2 (MAGEC2); MAGE family member D1 (MAGED1); MAGE family member D2 (MAGED2); mesothelin (MSLN); mucin 1 (MUC1) and splice variants thereof (e.g., MUC1/C, D, and Z); mucin 16 (MUC16); necdin (NDN); nectin cell adhesion molecule 4 (NECTIN4); SLIT and NTRK like family member 6 (SLITRK6); promyelocytic leukemia (PML, TRIM19); protein tyrosine kinase 7 (inactive) (PTK7); SLAM family member 6 (SLAMF6, CD352); SLAM family member 7 (SLAMF7, 19A, CD319, CRACC, CS1); sialic acid binding Ig like lectin 7 (SIGLEC7); sialic acid binding Ig like lectin 9 (SIGLEC9); solute carrier family 34 (sodium phosphate), member 2 (SLC34A2); solute carrier family 39 member 6 (SLC39A6; LIV1); STEAP family member 1 (STEAP1); TNF receptor superfamily member 4 (TNFRSF4, OX40 or CD134); TNF superfamily member 9 (TNFSF9; 4-1BB-L, CD137L); TNF receptor superfamily member 10a (TNFRSF10A, DR4, CD261, TRAILR1); TNF receptor superfamily member 10b (TNFRSF10B, DR5, CD262, TRAILR2); TNF receptor superfamily member 13B (TNFRSF13B; CD267, TACI, IGAD2); TNF receptor superfamily member 17 (TNFRSF17, BCMA, CD269); TNF receptor superfamily member 18 (TNFRSF18, GITR or CD357); transferrin (TF); transforming growth factor beta 1 (TGFB1); trophoblast glycoprotein (TPBG, 5T4); trophinin (TRO, MAGED3); tumor associated calcium signal transducer 2 (TACSTD2, TROP2, EGP1); Fucosyl GM1; sialyl Lewis adhesion molecule (sLe); and Lewis Y antigen.

758. The method of any one of embodiments 752-757, wherein the cells in the cellular therapy bind to an epitope of a target or tumor associated antigen (TAA) presented in a major histocompatibility complex (MHC) molecule.

759. The method of any one of embodiments 750, 751, and 758, wherein the TAA is a cancer testis antigen.

760. The method of embodiment 759, wherein the cancer testis antigen is selected from the group consisting of acrosin binding protein (ACRBP), alpha fetoprotein (AFP), A-kinase anchoring protein 4 (AKAP4), ATPase family AAA domain containing 2 (ATAD2), kinetochore scaffold 1 (KNL1; a.k.a., CASC5), centrosomal protein 55 (CEP55), cancer/testis antigen 1A (CTAG1A; a.k.a., ESO1; CT6.1; LAGE-2; LAGE2A; NY-ESO-1), cancer/testis antigen 1B (CTAG1B; a.k.a., CT6.1, CTAG, CTAG1, ESO1, LAGE-2, LAGE2B, NY-ESO-1), cancer/testis antigen 2 (CTAG2; a.k.a., CAMEL, CT2, CT6.2, CT6.2a, CT6.2b, ESO2, LAGE-1, LAGE2B), CCCTC-binding factor like (CTCFL), catenin alpha 2 (CTNNA2), cancer/testis antigen 83 (CT83), cyclin A1 (CCNA1), DEAD-box helicase 43 (DDX43), developmental pluripotency associated 2 (DPPA2), fetal and adult testis expressed 1 (FATE1), FMR1 neighbor (FMR1NB), HORMA domain containing 1 (HORMAD1), insulin like growth factor 2 mRNA binding protein 3 (IGF2BP3), leucine zipper protein 4 (LUZP4), lymphocyte antigen 6 family member K (LY6K), maelstrom spermatogenic transposon silencer (MAEL), MAGE family member A1 (MAGEA1); MAGE family member A3 (MAGEA3); MAGE family member A4 (MAGEA4); MAGE family member A11 (MAGEA11); MAGE family member C1 (MAGEC1); MAGE family member C2 (MAGEC2); MAGE family member D1 (MAGED1); MAGE family member D2 (MAGED2), kinesin family member 20B (KIF20B; a.k.a., MPHOSPH1), NUF2 component of NDC80 kinetochore complex (NUF2), nuclear RNA export factor 2 (NXF2), PAS domain containing repressor 1 (PASD1), PDZ binding kinase (PBK), piwi like RNA-mediated gene silencing 2 (PIWIL2), preferentially expressed antigen in melanoma (PRAME), sperm associated antigen 9 (SPAG9), sperm protein associated with the nucleus, X-linked, family member A1 (SPANXA1), SPANX family member A2 (SPANXA2), SPANX family member C (SPANXC), SPANX family member D (SPANXD), SSX family member 1 (SSX1), SSX family member 2 (SSX2), synaptonemal complex protein 3 (SYCP3), testis expressed 14, intercellular bridge forming factor (TEX14), transcription factor Dp family member 3 (TFDP3), serine protease 50 (PRSS50, a.k.a., TSP50), TTK protein kinase (TTK) and zinc finger protein 165 (ZNF165).

761. The method of embodiment 750, wherein the non-immunoglobulin antigen-binding domains or antibody mimetic proteins are selected from the group consisting of adnectins, affibody molecules, affilins, affimers, affitins, alphabodies, anticalins, peptide aptamers, armadillo repeat proteins (ARMs), atrimers, avimers, designed ankyrin repeat proteins (DARPins®), fynomers, knottins, Kunitz domain peptides, monobodies, and nanoCLAMPs.

762. The method of any one of embodiments 181-200, 401-421, 621-640, and 748, wherein the immunotherapy comprises co-administering one or more antagonists or inhibitors of an inhibitory immune checkpoint protein or receptor and/or one or more activators or agonists of a stimulatory immune checkpoint protein or receptor.

763. The method of embodiment 762, wherein the immune checkpoint inhibitor is a small molecule inhibitor of CD274 (PDL1, PD-L1), programmed cell death 1 (PDCD1, PD1, PD-1) or CTLA4.

764. The method of embodiment 763, wherein the small molecule inhibitor of CD274 or PDCD1 is selected from the group consisting of GS-4224, GS-4416, INCB086550 and MAX10181.

765. The method of any one of embodiments 181-200, 401-421, 621-640, and 748, wherein the immunotherapy comprises co-administering one or more agents that selectively deplete suppressive myeloid cells.

766. The method of embodiment 765, wherein the suppressive myeloid cells are selected from tumor-associated macrophages (TAM) and myeloid derived suppressor cells (MDSC).

767. The method of embodiment 765 or 766, wherein the one or more agents that selectively deplete suppressive myeloid cells comprise an antibody or antigen-binding fragment thereof that selectively binds to a cell surface receptor selected from the group consisting of colony stimulating factor 1 receptor (CSF1R), C-C motif chemokine receptor 2 (CCR2), C-C motif chemokine ligand 2 (CCL2), triggering receptor expressed on myeloid cells 2 (TREM2), complement C5a receptor 1 (C5AR1) and combinations thereof.

768. The method of embodiment 767, wherein the cytokine or chemokine therapy comprises co-administering one or more immunostimulatory cytokines or chemokines that promote or increase the proliferation or activation of T cells (including alpha/beta TCR T cells and gamma/delta TCR T cells), NK-T cells, NK cells, and/or dendritic cells.

769. The method of embodiment 768, wherein the one or more immunostimulatory cytokines or chemokines are selected from the group consisting of: IL-2, IL-12, IL-15, IL-18, IL-21, interferon (IFN)-α, IFN-β, IFN-γ, CXCL9/Mig (monokine induced by interferon-γ), CXCL10/IP10 (interferon-γ-inducible 10 kDa protein) and CXCL11/I-TAC (interferon-inducible T cell α-chemoattractant), CXCL4/PF4 (platelet factor 4), monocyte chemoattractant protein 2 (MCP-2), macrophage inflammatory protein 1 alpha (MIP-1a), macrophage inflammatory protein 1 beta (MIP-1β) and regulated on activation normal T expressed and secreted protein (RANTES).

770. The method of any one of embodiments 181-200, 401-420, 621-640, and 748, wherein the immunotherapy comprises co-administering an immune checkpoint protein or receptor.

771. The method of embodiment 770, wherein the immune checkpoint protein or receptor is selected from the group consisting of: CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAET1E; ULBP4); retinoic acid early transcript 1G (RAET1G; ULBP5); retinoic acid early transcript 1L (RAET1L; ULBP6); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); killer cell lectin like receptor G1 (KLRG1; CLEC15A, MAFA, 2F1); sialic acid binding Ig like lectin 7 (SIGLEC7); and sialic acid binding Ig like lectin 9 (SIGLEC9).

772. The method of any one of embodiments 181-200, 401-420, 621-640, and 748, wherein the immunotherapy comprises co-administering one or more blockers or inhibitors of a T-cell inhibitory immune checkpoint protein or receptor.

773. The method of embodiment 772, wherein the T-cell inhibitory immune checkpoint protein or receptor is selected from the group consisting of CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1).

774. The method of any one of embodiments 181-200, 401-420, 621-640, and 748, wherein the immunotherapy comprises co-administering one or more agonists or activators of one or more T-cell stimulatory immune checkpoint proteins or receptors.

775. The method of embodiment 774, wherein the T-cell stimulatory immune checkpoint proteins or receptors are selected from the group consisting of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155).

776. The method of any one of embodiments 1-100, 161-320, 381-540, and 601-775, further comprising co-administering to the subject sacituzumab govitecan.

777. The method of any one of embodiments 1-100, 161-320, 381-540, and 601-775, further comprising co-administering to the subject an anti-CD47 antibody.

778. The method of embodiment 777, wherein the anti-CD47 antibody is magrolimab.

779. The method of any one of embodiments 1-100, 161-320, 381-540, and 601-775, further comprising co-administering to the subject an inhibitor of MCL-1.

780. The method of any one of embodiments 141-160, 361-380, 581-600, and 779, wherein the inhibitor of MCL1 is selected from the group consisting of GS-9716, AMG-176, AMG-397, S-64315, AZD-5991, 483-LM, A-1210477, UMI-77 and JKY-5-037.

781. The method of any one of embodiments 1-780, wherein the fusion protein is co-administered with one or more therapeutic agents selected from the group consisting of AGEN1884 (zalifrelimab), AGEN1181, AGEN2034 (balstilimab), AGEN1307, AGEN2373, AGEN1223 and GS-1423 (AGEN1423).

782. The method of any one of embodiments 1-781, wherein the fusion protein is co-administered with a vaccine.

783. The method of embodiment 782, wherein the vaccine is selected from the group consisting of an antiviral vaccine, an antibacterial vaccine and an anticancer vaccine.

784. The method of embodiment 782, wherein the vaccine comprises an antiviral vaccine against a virus selected from the group consisting of hepatitis A virus (HAV), hepatitis B virus (HBV), human immunodeficiency virus (HIV), cytomegalovirus (CMV), a herpes simplex virus (HSV), Epstein-Barr virus (EBV), human orthopneumovirus or human respiratory syncytial virus (RSV), human papillomavirus (HPV), varicella-zoster virus, measles virus, mumps virus, poliovirus vaccine, influenza virus, paramyxovirus, rotavirus, Zika virus, Dengue virus and Ebola virus.

785. The method of embodiment 782, wherein the vaccine comprises an antibacterial vaccine against a bacterium selected from the group consisting of Mycobacterium tuberculosis, pertussis, tetanus, diphtheria, meningococcus, pneumococcus, Haemophilus influenza, cholera, typhoid, and anthrax.

786. The method of any one of embodiments 1-785, wherein the fusion protein is co-administered with an oncolytic viral vector.

787. The method of embodiment 786, wherein the oncolytic viral vector comprises a DNA virus or an RNA virus.

788. The method of embodiment 786 or 787, wherein the viral vector is from a viral family selected from the group consisting of: Adenoviridae (e.g., Adenovirus), Arenaviridae (e.g., lymphocytic choriomeningitis mammarenavirus, Cali mammarenavirus (a.k.a., Pichinde mammarenavirus), Poxviridae (e.g., Vaccinia virus), Herpesviridae (e.g., Herpesvirus, e.g., HSV-1), Parvoviridae (e.g., Parvovirus H1), Reoviridae (e.g., Reovirus), Picornaviridae (e.g., Coxsackievirus, Seneca Valley Virus, Poliovirus), Paramyxoviridae (e.g., Measles virus, Newcastle disease virus (NDV)), Rhabdoviridae (e.g., Vesicular stomatitis virus (VSV)), Togaviridae (e.g., Alphavirus, Sindbis virus), Enteroviridae (e.g., Echovirus).

789. The method of any one of embodiments 1-788, wherein the fusion protein is co-administered with an immunostimulatory therapy, a cytokine therapy, a chemokine therapy, a cellular therapy, a gene therapy, and combinations thereof.

790. The method of any one of embodiments 1-789, wherein the fusion protein is co-administered with a FOLFOX regimen, a FOLFIRI regimen, a FOLFOXIRI regimen or a FOLFIRINOX regimen.

791. The method of any one of embodiments 1-790, wherein the fusion protein is co-administered with a targeted E3 ligase ligand conjugate.

792. The method of any one of embodiments 1-791, wherein the fusion protein is co-administered with one or more additional therapeutic agents comprising an activator or agonist of:
a toll-like receptor (TLR);
a stimulator of interferon genes (STING) receptor;
inducible T cell costimulator (ICOS, CD278); and/or
a TNF receptor superfamily (TNFRSF) member.

793. The method of embodiment 792, wherein the TNF receptor superfamily (TNFRSF) member is selected from the group consisting of: TNFRSF1A, TNFRSF1B, TNFRSF4 (OX40), TNFRSF5 (CD40), TNFRSF6 (FAS), TNFRSF7 (CD27), TNFRSF8 (CD30), TNFRSF9 (4-1BB, CD137), TNFRSF10A (CD261, DR4, TRAILR1), TNFRSF10B (CD262, DR5, TRAILR2), TNFRSF10C (CD263, TRAILR3), TNFRSF10D (CD264, TRAILR4), TNFRSF11A (CD265, RANK), TNFRSF11B, TNFRSF12A (CD266), TNFRSF13B (CD267), TNFRSF13C (CD268), TNFRSF16 (NGFR, CD271), TNFRSF17 (BCMA, CD269), TNFRSF18 (GITR, CD357), TNFRSF19, TNFRSF21 (CD358, DR6), and TNFRSF25 (DR3).

794. The method of embodiment 793, wherein:
the TNFRSF4 (OX40 or CD134) activator or agonist comprises INCAGN1949, tavolimab (MEDI0562), pogalizumab (MOXR0916/RG7888), MEDI6469, BMS-986178, PF-04518600, GSK3174998, IBI101, ATOR-1015, ABBV-368 or SL-279252;
the TNFRSF9 (4-1BB or CD137) activator or agonist comprises urelumab, BMS-663513, utomilumab (PF-05082566), CTX-471, MP-0310, ADG-106, ATOR-1017 or AGEN2373; and/or
the TNFRSF18 (GITR or CD357) activator or agonist comprises GWN323, MEDI1873, MK-1248, MK-4166, TRX518, INCAGN1876, BMS-986156, BMS-986256, AMG-228, ASP1951 (PTZ 522), FPA-154 or OMP-336B11.

795. The method of any one of embodiments 792-794, comprising co-administering a molecule that concurrently binds to TNF receptor superfamily member 4 (TNFRSF4, OX40 or CD134) and TNF receptor superfamily member 18 (TNFRSF18, GITR or CD357).

796. The method of embodiment 792, wherein the TLR agonist or activator is selected from the group consisting of a TLR2 agonist, a TLR3 agonist, a TLR4 agonist, a TLR5 agonist, a TLR7 agonist, a TLR8 agonist and a TLR9 agonist.

797. The method of any one of embodiments 1-796, wherein the fusion protein is co-administered with a SIRPα targeting agent.

798. The method of embodiment 797, wherein the SIRPα targeting agent is selected from the group consisting of AL-008, RRx-001, CTX-5861, FSI-189 (GS-0189), ES-004, BI765063, ADU1805, and CC-95251.

799. The method of any one of embodiments 1-798, wherein the fusion protein is co-administered with one or more additional therapeutic agents comprising an inhibitor or antagonist of:
protein tyrosine phosphatase, non-receptor type 11 (PTPN11 or SHP2),
myeloid cell leukemia sequence 1 (MCL1) apoptosis regulator,
mitogen-activated protein kinase kinase kinase kinase 1 (MAP4K1) (also called Hematopoietic Progenitor Kinase 1 (HPK1)),
phosphatidylinositol-4,5-bisphosphate 3-kinase, including catalytic subunit alpha (PIK3CA), catalytic subunit beta (PIK3CB), catalytic subunit gamma (PIK3CG) and catalytic subunit delta (PIK3CD),
diacylglycerol kinase alpha (DGKA, DAGK, DAGK1 or DGK-alpha),
diacylglycerol kinase alpha (DGKA, DAGK, DAGK1 or DGK-alpha),
5′-nucleotidase ecto (NTSE or CD73),
ectonucleoside triphosphate diphosphohydrolase 1 (ENTPD1 or CD39),
transforming growth factor beta 1 (TGFB1 or TGFβ),
heme oxygenase 1 (HMOX1, HO-1 or HO1),
heme oxygenase 2 (HMOX2, HO-2 or HO2),
vascular endothelial growth factor A (VEGFA or VEGF),
erb-b2 receptor tyrosine kinase 2 (ERBB2, HER2, HER2/neu or CD340),
epidermal growth factor receptor (EGFR, ERBB, ERBB1 or HER1),
ALK receptor tyrosine kinase (ALK, CD246),
poly(ADP-ribose) polymerase 1 (PARP1),
poly(ADP-ribose) polymerase 2 (PARP2),
TCDD inducible poly(ADP-ribose) polymerase (TIPARP, PARP7),
cyclin dependent kinase 4 (CDK4),
cyclin dependent kinase 6 (CDK6),
TNF receptor superfamily member 14 (TNFRSF14, HVEM, CD270),
T cell immunoreceptor with Ig and ITIM domains (TIGIT),
X-linked inhibitor of apoptosis (XIAP, BIRC4, IAP-3),
baculoviral IAP repeat containing 2 (BIRC2, cIAP1),
baculoviral IAP repeat containing 3 (BIRC3, cIAP2),
baculoviral IAP repeat containing 5 (BIRC5, survivin),
C-C motif chemokine receptor 2 (CCR2, CD192),
C-C motif chemokine receptor 5 (CCR5, CD195),
C-C motif chemokine receptor 8 (CCR8, CDw198),
C-X-C motif chemokine receptor 2 (CXCR2, CD182),
C-X-C motif chemokine receptor 3 (CXCR3, CD182, CD183),
C-X-C motif chemokine receptor 4 (CXCR4, CD184),
cytokine inducible SH2 containing protein (CISH),
arginase (ARG1, ARG2),
carbonic anhydrase (CA1, CA2, CA3, CA4, CA5A, CA5B, CA6, CA7, CA8, CA9, CA10, CA11, CA12, CA13, CA14),
prostaglandin-endoperoxide synthase 1 (PTGS1, COX-1),
prostaglandin-endoperoxide synthase 2 (PTGS2, COX-2),
secreted phospholipase A2,
prostaglandin E synthase (PTGES, PGES),
arachidonate 5-lipoxygenase (ALOX5, 5-LOX),
soluble epoxide hydrolase 2 (EPHX2),
indoleamine 2,3-dioxygenase 1 (IDO1),
indoleamine 2,3-dioxygenase 2 (IDO2),
hypoxia inducible factor 1 subunit alpha (HIF1A),
angiopoietin 1 (ANGPT1),
Endothelial TEK tyrosine kinase (TIE-2, TEK),
Janus kinase 1 (JAK1),
catenin beta 1 (CTNNB1),
histone deacetylase 9 (HDAC9),
5′-3′ exoribonuclease 1 (XRN1), and/or
WRN RecQ like helicase (WRN).

800. The method of embodiment 799, wherein the inhibitor comprises an antibody or an antigen-binding fragment thereof, or antibody-drug conjugate thereof, CD3-targeting multi-specific molecule, NK cell-activating receptor-targeting multi-specific molecule, non-immunoglobulin antigen binding molecule or antibody mimetic protein.

801. The method of embodiment 800, wherein the NK cell-activating receptor is selected from the group consisting of CD16, NKp30, NKp44, NKp46, NKp80 and NKG2D.

802. The method of embodiment 799, wherein the inhibitor comprises an inhibitory nucleic acid.

803. The method of embodiment 799, wherein the inhibitor comprises a small organic molecule.

804. The method of any one of embodiments 799, 802, and 803, wherein the inhibitor of 5′-nucleotidase ecto (NT5E or CD73) is selected from the group consisting of MEDI9447 (oleclumab), CPI-006, BMS-986179, IPH5301, TJ4309 (TJD5), NZV-930, AB-680, PSB-12379, PSB-12441, PSB-12425, CB-708, GS-1423 (AGEN-1423) and PBF-1662.

805. The method of any one of embodiments 799, 802, and 803, wherein the inhibitor of CCR2 and/or CCR5 is selected from the group consisting of BMS-813160, PF-04136309 and CCX-872.

806. The method of any one of embodiments 799,802, and 803, wherein the inhibitor of PTPN11 or SHP2 is selected from the group consisting of TNO155 (SHP-099), RMC-4550, JAB-3068 and RMC-4630.

807. The method of any one of embodiments 799,802, and 803, wherein the inhibitor of Janus kinase 1 (JAK1) is selected from the group consisting of filgotinib, tofacitinib, baricitinib and ABT-494.

808. The method of any one of embodiments 1-807, wherein the fusion protein is co-administered with one or more additional therapeutic agents comprising a regulatory T-cell (Treg) inhibitor.

809. The method of embodiment 808, wherein the Treg inhibitor comprises an antibody or antigen-binding fragment thereof that selectively binds to a cell surface receptor selected from the group consisting of C-C motif chemokine receptor 4 (CCR4), C-C motif chemokine receptor 7 (CCR7), C-C motif chemokine receptor 8 (CCR8), C-X-C motif chemokine receptor 4 (CXCR4; CD184), TNFRSF4 (OX40), TNFRSF18 (GITR, CD357), TNFRSF9 (4-1BB, CD137), cytotoxic T-lymphocyte associated protein 4 (CTLA4, CD152), programmed cell death 1 (PDCD1, PD-1), Sialyl Lewis x (CD15s), CD27, ectonucleoside triphosphate diphosphohydrolase 1 (ENTPD1; CD39), protein tyrosine phosphatase receptor type C (PTPRC; CD45), neural cell adhesion molecule 1 (NCAM1; CD56), selectin L (SELL; CD62L), integrin subunit alpha E (ITGAE; CD103), interleukin 7 receptor (IL7R; CD127), CD40 ligand (CD40LG; CD154), folate receptor alpha (FOLR1), folate receptor beta (FOLR2), leucine rich repeat containing 32 (LRRC32; GARP), IKAROS family zinc finger 2 (IKZF2; HELIOS), inducible T cell costimulatory (ICOS; CD278), lymphocyte activating 3 (LAG3; CD223), transforming growth factor beta 1 (TGFB1), hepatitis A virus cellular receptor 2 (HAVCR2; CD366; TIM3), T cell immunoreceptor with Ig and ITIM domains (TIGIT), TNF receptor superfamily member 1B (CD120b; TNFR2), IL2RA (CD25) and combinations thereof.

810. The method of any one of embodiment 1-809, further wherein the subject receives radiation therapy.

811. The method of embodiment 810, where the radiation therapy comprises stereotactic body radiation therapy (SBRT).

812. The method of any one of embodiments 81-160, 181-200, 301-380, 401-420, 521-600, 621-640, and 740-811, wherein the fusion protein is administered prior to co-administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy.

813. The method of embodiment 812, wherein the fusion protein is administered at least 1, 2, 3, 4, 5, 6, 7, 8, 9, or 10 days prior to administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy.

814. The method of any one of embodiments 81-160, 181-200, 301-380, 401-420, 521-600, 621-640, and 740-811, wherein the fusion protein is administered after administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy.

815. The method of embodiment 814, wherein the fusion protein is administered at least 1, 2, 3, 4, 5, 6, 7, 8, 9, or 10 days after administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy.

816. The method of any one of embodiments 81-160, 181-200, 301-380, 401-420, 521-600, 621-640, and 740-811, wherein the fusion protein is administered concurrently with administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy.

817. The method of embodiment 816, wherein the fusion protein is administered within 10, 15, 20, 25, 30, 35, 40, 45, 50, 55, 60, 65, 70, 75, 80, 85, 90, 95, 100, 110, 120, 130, 140, 150, 160, 170, 180, 190, or 200 minutes of administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy.

818. The method of embodiment 816, wherein the fusion protein is administered within 1, 2, 3, 4, 5, 6, 7, 8, 9, 10, 11, 12, 13, 14, or 15 hours of administration of the anticancer agent, immunotherapy, sacituzumab govitecan, anti-CD47 antibody, magrolimab, inhibitor of MCL-1, therapeutic agent, vaccine, oncolytic viral vector, immunostimulatory therapy, cytokine therapy, chemokine therapy, cellular therapy, gene therapy, targeted E3 ligase ligand conjugate, SIRPα targeting agent, and/or radiation therapy.

819. The method of any one of embodiments 1-818, wherein the subject has cancer.

820. The method of any one of embodiments 1-818, wherein the subject is in cancer remission.

821. The method of any one of embodiments 1-818, wherein the subject has a hematological cancer, e.g., a leukemia (e.g., Acute Myelogenous Leukemia (AML), Acute Lymphoblastic Leukemia (ALL), B-cell ALL, Myelodysplastic Syndrome (MDS), myeloproliferative disease (MPD), Chronic Myelogenous Leukemia (CIVIL), Chronic Lymphocytic Leukemia (CLL), undifferentiated leukemia), a lymphoma (e.g., small lymphocytic lymphoma (SLL), mantle cell lymphoma (MCL), follicular lymphoma (FL), T-cell lymphoma, B-cell lymphoma, diffuse large B-cell lymphoma (DLBCL), marginal zone lymphoma (MZL), Waldestrom's macroglobulinemia (WM)) and/or a myeloma (e.g., multiple myeloma (MM)).

822. The method of any one of embodiments 1-818, wherein the subject has a solid tumor.

823. The method of embodiment 822, wherein the solid tumor is a malignant tumor.

824. The method of embodiment 822 or 823, wherein the solid tumor is a metastatic tumor.

825. The method of any one of embodiments 1-824, wherein the subject has a tumor infiltrated with conventional dendritic cells (cDC1).

826. The methods of embodiment 825, wherein the tumor infiltrating dendritic cells express C-C motif chemokine receptor 5 (CCR5, CD195) and/or X-C motif chemokine receptor 1 (XCR1) on their cell surface.

827. The methods of embodiment 825, wherein the tumor infiltrating dendritic cells express one or more cell surface proteins selected from the group consisting of XCR1, cell adhesion molecule 1 (CADM1), C-type lectin domain containing 9A (CLEC9A, CD370), and thrombomodulin (THBD).

828. The methods of embodiment 825, wherein the tumor infiltrating dendritic cells express one or more cell surface proteins selected from the group consisting of CD1A, CD1C, CD1E, signal regulatory protein alpha (SIRPA; CD172A), CD207 and Fc fragment of IgE receptor Ia (FCER1A).

829. The methods of embodiment 825, wherein the tumor infiltrating dendritic cells express one or more proteins selected from the group consisting of basic leucine zipper ATF-like transcription factor 3 (BATF3) and interferon regulatory factor 8 (IRF8).

830. The methods of embodiment 825, wherein the tumor infiltrating dendritic cells express one or more proteins selected from the group consisting of BATF3, IRF8, THBD, CLEC9A and XCR1.

831. The method of any one of embodiments 1-830, wherein the subject has a cancer that detectably expresses or overexpresses one or more cell surface immune checkpoint receptors.

832. The method of embodiment 831, wherein the one or more cell surface immune checkpoint receptors are selected from the group consisting of: CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAET1E; ULBP4); retinoic acid early transcript 1G (RAET1G; ULBP5); retinoic acid early transcript 1L (RAET1L; ULBP6); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); killer cell lectin like receptor G1 (KLRG1; CLEC15A, MAFA, 2F1); sialic acid binding Ig like lectin 7 (SIGLEC7); and sialic acid binding Ig like lectin 9 (SIGLEC9).

833. The method of embodiment 831 or 832, wherein greater than about 50% of the cancer cells detectably express one or more cell surface immune checkpoint receptors (e.g., so-called “hot” cancer or tumor).

834. The method of embodiment 831 or 832, wherein greater than about 1% and less than about 50% of the cancer cells detectably express one or more cell surface immune checkpoint receptors (e.g., so called “warm” cancer or tumor).

835. The method of embodiment 831 or 832, wherein less than about 1% of the cancer cells detectably express one or more cell surface immune checkpoint receptors (e.g., so called “cold” cancer or tumor).

836. The method of any one of embodiments 1-811, wherein the subject has a cancer selected from the group consisting of an epithelial tumor (e.g., a carcinoma, a squamous cell carcinoma, a basal cell carcinoma, a squamous intraepithelial neoplasia), a glandular tumor (e.g., an adenocarcinoma, an adenoma, an adenomyoma), a mesenchymal or soft tissue tumor (e.g., a sarcoma, a rhabdomyosarcoma, a leiomyosarcoma, a liposarcoma, a fibrosarcoma, a dermatofibrosarcoma, a neurofibrosarcoma, a fibrous histiocytoma, an angiosarcoma, an angiomyxoma, a leiomyoma, a chondroma, a chondrosarcoma, an alveolar soft-part sarcoma, an epithelioid hemangioendothelioma, a Spitz tumor, a synovial sarcoma), and a lymphoma.

837. The method of any one of embodiments 1-811, wherein the subject has a solid tumor in or arising from a tissue or organ selected from the group consisting of:
bone (e.g., adamantinoma, aneurysmal bone cysts, angiosarcoma, chondroblastoma, chondroma, chondromyxoid fibroma, chondrosarcoma, chordoma, dedifferentiated chondrosarcoma, enchondroma, epithelioid hemangioendothelioma, fibrous dysplasia of the bone, giant cell tumour of bone, haemangiomas and related lesions, osteoblastoma, osteochondroma, osteosarcoma, osteoid osteoma, osteoma, periosteal chondroma, Desmoid tumor, Ewing sarcoma);
lips and oral cavity (e.g., odontogenic ameloblastoma, oral leukoplakia, oral squamous cell carcinoma, primary oral mucosal melanoma); salivary glands (e.g., pleomorphic salivary gland adenoma, salivary gland adenoid cystic carcinoma, salivary gland mucoepidermoid carcinoma, salivary gland Warthin's tumors);
esophagus (e.g., Barrett's esophagus, dysplasia and adenocarcinoma);
gastrointestinal tract, including stomach (e.g., gastric adenocarcinoma, primary gastric lymphoma, gastrointestinal stromal tumors (GISTs), metastatic deposits, gastric carcinoids, gastric sarcomas, neuroendocrine carcinoma, gastric primary squamous cell carcinoma, gastric adenoacanthomas), intestines and smooth muscle (e.g., intravenous leiomyomatosis), colon (e.g., colorectal adenocarcinoma), rectum, anus;
pancreas (e.g., serous neoplasms, including microcystic or macrocystic serous cystadenoma, solid serous cystadenoma, Von Hippel-Landau (VHL)-associated serous cystic neoplasm, serous cystadenocarcinoma; mucinous cystic neoplasms (MCN), intraductal papillary mucinous neoplasms (IPMN), intraductal oncocytic papillary neoplasms (IOPN), intraductal tubular neoplasms, cystic acinar neoplasms, including acinar cell cystadenoma, acinar cell cystadenocarcinoma, pancreatic adenocarcinoma, invasive pancreatic ductal adenocarcinomas, including tubular adenocarcinoma, adenosquamous carcinoma, colloid carcinoma, medullary carcinoma, hepatoid carcinoma, signet ring cell carcinoma, undifferentiated carcinoma, undifferentiated carcinoma with osteoclast-like giant cells, acinar cell carcinoma, neuroendocrine neoplasms, neuroendocrine microadenoma, neuroendocrine tumors (NET), neuroendocrine carcinoma (NEC), including small cell or large cell NEC, insulinoma, gastrinoma, glucagonoma, serotonin-producing NET, somatostatinoma, VIPoma, solid-pseudopapillary neoplasms (SPN), pancreatoblastoma);
gall bladder (e.g., carcinoma of the gallbladder and extrahepatic bile ducts, intrahepatic cholangiocarcinoma);
neuro-endocrine (e.g., adrenal cortical carcinoma, carcinoid tumors, phaeochromocytoma, pituitary adenomas);
thyroid (e.g., anaplastic (undifferentiated) carcinoma, medullary carcinoma, oncocytic tumors, papillary carcinoma, adenocarcinoma);
liver (e.g., adenoma, combined hepatocellular and cholangiocarcinoma, fibrolamellar carcinoma, hepatoblastoma, hepatocellular carcinoma, mesenchymal, nested stromal epithelial tumor, undifferentiated carcinoma;
hepatocellular carcinoma, intrahepatic cholangiocarcinoma, bile duct cystadenocarcinoma, epithelioid hemangioendothelioma, angiosarcoma, embryonal sarcoma, rhabdomyosarcoma, solitary fibrous tumor, teratoma, York sac tumor, carcinosarcoma, rhabdoid tumor);
kidney (e.g., ALK-rearranged renal cell carcinoma, chromophobe renal cell carcinoma, clear cell renal cell carcinoma, clear cell sarcoma, metanephric adenoma, metanephric adenofibroma, mucinous tubular and spindle cell carcinoma, nephroma, nephroblastoma (Wilms tumor), papillary adenoma, papillary renal cell carcinoma, renal oncocytoma, renal cell carcinoma, succinate dehydrogenase-deficient renal cell carcinoma, collecting duct carcinoma);
breast (e.g., invasive ductal carcinoma, including without limitation, acinic cell carcinoma, adenoid cystic carcinoma, apocrine carcinoma, cribriform carcinoma, glycogen-rich/clear cell, inflammatory carcinoma, lipid-rich carcinoma, medullary carcinoma, metaplastic carcinoma, micropapillary carcinoma, mucinous carcinoma, neuroendocrine carcinoma, oncocytic carcinoma, papillary carcinoma, sebaceous carcinoma, secretory breast carcinoma, tubular carcinoma; lobular carcinoma, including without limitation, pleomorphic carcinoma, signet ring cell carcinoma);
peritoneum (e.g., mesothelioma; primary peritoneal cancer);
female sex organ tissues, including ovary (e.g., choriocarcinoma, epithelial tumors, germ cell tumors, sex cord-stromal tumors), Fallopian tubes (e.g., serous adenocarcinoma, mucinous adenocarcinoma, endometrioid adenocarcinoma, clear cell adenocarcinoma, transitional cell carcinoma, squamous cell carcinoma, undifferentiated carcinoma, Müllerian tumors, adenosarcoma, leiomyosarcoma, teratoma, germ cell tumors, choriocarcinoma, trophoblastic tumors), uterus (e.g., carcinoma of the cervix, endometrial polyps, endometrial hyperplasia, intraepithelial carcinoma (EIC), endometrial carcinoma (e.g., endometrioid carcinoma, serous carcinoma, clear cell carcinoma, mucinous carcinoma, squamous cell carcinoma, transitional carcinoma, small cell carcinoma, undifferentiated carcinoma, mesenchymal neoplasia), leiomyoma (e.g., endometrial stromal nodule, leiomyosarcoma, endometrial stromal sarcoma (ESS), mesenchymal tumors), mixed epithelial and mesenchymal tumors (e.g., adenofibroma, carcinofibroma, adenosarcoma, carcinosarcoma (malignant mixed mesodermal sarcoma—MMMT)), endometrial stromal tumors, endometrial malignant mullerian mixed tumours, gestational trophoblastic tumors (partial hydatiform mole, complete hydatiform mole, invasive hydatiform mole, placental site tumour)), vulva, vagina;
male sex organ tissues, including prostate, testis (e.g., germ cell tumors, spermatocytic seminoma), penis;
bladder (e.g., squamous cell carcinoma, urothelial carcinoma, bladder urothelial carcinoma);
brain, (e.g., gliomas (e.g., astrocytomas, including non-infiltrating, low-grade, anaplastic, glioblastomas; oligodendrogliomas, ependymomas), meningiomas, gangliogliomas, schwannomas (neurilemmomas), craniopharyngiomas, chordomas, Non-Hodgkin lymphomas (NHLs), indolent non-Hodgkin's lymphoma (iNHL), refractory iNHL, pituitary tumors;
eye (e.g., retinoma, retinoblastoma, ocular melanoma, posterior uveal melanoma, iris hamartoma);
head and neck (e.g., nasopharyngeal carcinoma, Endolymphatic Sac Tumor (ELST), epidermoid carcinoma, laryngeal cancers including squamous cell carcinoma (SCC) (e.g., glottic carcinoma, supraglottic carcinoma, subglottic carcinoma, transglottic carcinoma), carcinoma in situ, verrucous, spindle cell and basaloid SCC, undifferentiated carcinoma, laryngeal adenocarcinoma, adenoid cystic carcinoma, neuroendocrine carcinomas, laryngeal sarcoma), head and neck paragangliomas (e.g., carotid body, jugulotympanic, vagal);
thymus (e.g., thymoma);
heart (e.g., cardiac myxoma);
lung (e.g., small cell carcinoma (SCLC), non-small cell lung carcinoma (NSCLC), including squamous cell carcinoma (SCC), adenocarcinoma and large cell carcinoma, carcinoids (typical or atypical), carcinosarcomas, pulmonary blastomas, giant cell carcinomas, spindle cell carcinomas, pleuropulmonary blastoma);
lymph (e.g., lymphomas, including Hodgkin's lymphoma, non-Hodgkin's lymphoma (NHL), indolent non-Hodgkin's lymphoma (iNHL), refractory iNHL, Epstein-Barr virus (EBV)-associated lymphoproliferative diseases, including B cell lymphomas and T cell lymphomas (e.g., Burkitt lymphoma;
large B cell lymphoma, diffuse large B-cell lymphoma (DLBCL), mantle cell lymphoma, indolent B-cell lymphoma, low grade B cell lymphoma, fibrin-associated diffuse large cell lymphoma; primary effusion lymphoma;
plasmablastic lymphoma; extranodal NK/T cell lymphoma, nasal type;
peripheral T cell lymphoma, cutaneous T cell lymphoma, angioimmunoblastic T cell lymphoma; follicular T cell lymphoma; systemic T cell lymphoma), lymphangioleiomyomatosis);
central nervous system (CNS) (e.g., gliomas including astrocytic tumors (e.g., pilocytic astrocytoma, pilomyxoid astrocytoma, subependymal giant cell astrocytoma, pleomorphic xanthoastrocytoma, diffuse astrocytoma, fibrillary astrocytoma, gemistocytic astrocytoma, protoplasmic astrocytoma, anaplastic astrocytoma, glioblastoma (e.g., giant cell glioblastoma, gliosarcoma, glioblastoma multiforme) and gliomatosis cerebri), oligodendroglial tumors (e.g., oligodendroglioma, anaplastic oligodendroglioma), oligoastrocytic tumors (e.g., oligoastrocytoma, anaplastic oligoastrocytoma), ependymal tumors (e.g., subependymom, myxopapillary ependymoma, ependymomas (e.g., cellular, papillary, clear cell, tanycytic), anaplastic ependymoma), optic nerve glioma, and non-gliomas (e.g., choroid plexus tumors, neuronal and mixed neuronal-glial tumors, pineal region tumors, embryonal tumors, medulloblastoma, meningeal tumors, primary CNS lymphomas, germ cell tumors, Pituitary adenomas, cranial and paraspinal nerve tumors, stellar region tumors); neurofibroma, meningioma, peripheral nerve sheath tumors, peripheral neuroblastic tumours (including without limitation neuroblastoma, ganglioneuroblastoma, ganglioneuroma), trisomy 19 ependymoma);
neuroendocrine tissues (e.g., paraganglionic system including adrenal medulla (pheochromocytomas) and extra-adrenal paraganglia ((extra-adrenal) paragangliomas);
skin (e.g., clear cell hidradenoma, cutaneous benign fibrous histiocytomas, cylindroma, hidradenoma, melanoma (including cutaneous melanoma, mucosal melanoma), pilomatricoma, Spitz tumors); and
soft tissues (e.g., aggressive angiomyxoma, alveolar rhabdomyosarcoma, alveolar soft part sarcoma, angiofibroma, angiomatoid fibrous histiocytoma, synovial sarcoma, biphasic synovial sarcoma, clear cell sarcoma, dermatofibrosarcoma protuberans, desmoid-type fibromatosis, small round cell tumor, desmoplastic small round cell tumor, elastofibroma, embryonal rhabdomyosarcoma, Ewing's tumors/primitive neurectodermal tumors (PNET), extraskeletal myxoid chondrosarcoma, extraskeletal osteosarcoma, paraspinal sarcoma, inflammatory myofibroblastic tumor, lipoblastoma, lipoma, chondroid lipoma, liposarcoma/malignant lipomatous tumors, liposarcoma, myxoid liposarcoma, fibromyxoid sarcoma, lymphangioleiomyoma, malignant myoepithelioma, malignant melanoma of soft parts, myoepithelial carcinoma, myoepithelioma, myxoinflammatory fibroblastic sarcoma, undifferentiated sarcoma, pericytoma, rhabdomyosarcoma, non-rhabdomyosarcoma soft tissue sarcoma (NRSTS), soft tissue leiomyosarcoma, undifferentiated sarcoma, well-differentiated liposarcoma.

838. The method of any one of embodiments 1-811, wherein the subject has a cancer selected from the group consisting of a lung cancer, a colorectal cancer, a breast cancer, a prostate cancer, a cervical cancer and a head and neck cancer.

839. The method of any one of embodiments 1-811, wherein the subject has neutropenia or lymphopenia.

840. The method of any one of embodiments 1-839, wherein the subject has received a lymphodepleting chemotherapy regimen.

841. The method of any one of embodiments 1-839, wherein the subject is naïve to or has not received chemotherapy.

842. The method of any one of embodiments 1-841, wherein the subject has bone marrow cells, or is not depleted of bone marrow cells.

843. The method of any one of embodiments 1-842, wherein the subject does not have a mutation in the gene encoding the FLT3 receptor that causes or results in or is associated with cancer.

844. The method of any one of embodiments 201-220, 421-440, and 641-660, wherein the subject is suffering from a virus infection.

845. The method of embodiment 844, wherein the virus infection is caused by a virus selected from the group consisting of hepatitis B virus, human immunodeficiency virus (HIV), and coronavirus.

846. The method of embodiment 845, wherein the coronavirus is selected from the group consisting of Severe Acute Respiratory Syndrom (SARS)-associated virus, Middle East Respiratory Syndrom (MERS)-associated virus, and COVID-19 virus (SARS-CoV-2). 847. The method of any preceding embodiment, wherein between about 600 μg to about 30000 μg, about 600 μg to about 29000 μg, about 600 μg to about 28000 μg, about 600 μg to about 27000 μg, about 600 μg to about 26000 μg, about 600 μg to about 25000 μg, about 600 μg to about 24000 μg, about 600 μg to about 23000 μg, about 600 μg to about 22000 μg, about 600 μg to about 21000 μg, about 600 μg to about 20000 μg, about 600 μg to about 19000 μs, about 600 μg to about 18000 μg, about 600 μg to about 17000 μg, about 600 μg to about 16000 μg, about 600 μg to about 15000 μg, about 600 μg to about 14000 μg, about 600 μg to about 13000 μg, about 600 μg to about 12000 μg, about 600 μg to about 11000 μg, about 600 μg to about 10000 μg, about 1000 μg to about 30000 μg, about 1000 μg to about 29000 μg, about 1000 μg to about 28000 μg, about 1000 μg to about 27000 μg, about 1000 μg to about 26000 μg, about 1000 μg to about 25000 μg, about 1000 μg to about 24000 μg, about 1000 μg to about 23000 μg, about 1000 μg to about 22000 μg, about 1000 μg to about 21000 μg, about 1000 μg to about 20000 μg, about 1000 μg to about 19000 μg, about 1000 μg to about 18000 μs, about 1000 μg to about 17000 μg, about 1000 μg to about 16000 μg, about 1000 μg to about 15000 μg, about 1000 μg to about 14000 μg, about 1000 μg to about 13000 μg, about 1000 μg to about 12000 μg, about 1000 μg to about 11000 μg, about 1000 μg to about 10000 μg, about 2000 μg to about 30000 μg, about 2000 μg to about 29000 μg, about 2000 μg to about 28000 μs, about 2000 μg to about 27000 μg, about 2000 μg to about 26000 μg, about 2000 μg to about 25000 μg, about 2000 μg to about 24000 μg, about 2000 μg to about 23000 μg, about 2000 μg to about 22000 μg, about 2000 μg to about 21000 μg, about 2000 μg to about 20000 μg, about 2000 μg to about 19000 μg, about 2000 μg to about 18000 μg, about 2000 μg to about 17000 μs, about 2000 μg to about 16000 μg, about 2000 μg to about 15000 μg, about 2000 μg to about 14000 μg, about 2000 μg to about 13000 μg, about 2000 μg to about 12000 μg, about 2000 μg to about 11000 μg, about 2000 μg to about 10000 μg of the fusion protein is administered to the subject in a single dose.

848. The method of any preceding embodiment, wherein between about 1000 μg to about 22000 μg of the fusion protein is administered to the subject in a single dose.

849. The method of any preceding embodiment, wherein between about 1500 μg to about 22000 μg of the fusion protein is administered to the subject in a single dose.

850. The method of any preceding embodiment, wherein between about 2000 μg to about 22000 μg of the fusion protein is administered to the subject in a single dose.

851. The method of any preceding embodiment, wherein between about 2000 μg to about 20000 μg of the fusion protein is administered to the subject in a single dose.

852. The method of any preceding embodiment, wherein between about 1000 μg to about 20000 μg of the fusion protein is administered to the subject in a single dose.

853. The method of any preceding embodiment, wherein between about 1000 μg to about 22000 μg of the fusion protein is administered to the subject in a single dose.

854. The method of any preceding embodiment, wherein no more than about 29000 μg, 28000 μg, 27000 μg, 26000 μg, 25000 μg, 24000 μg, 23000 μg, 22000 μg, 21000 μg, 20000 μg, 19000 μg, 18000 μg, 17000 μg, 16000 μg, 15000 μg, 14000 μg, 13000 μg, 12000 μg, 11000 μg, 10000 μg, 9000 μg, 8000 μg, 7000 μg, 6000 μg, or 5000 μg of the fusion protein is administered to the subject in a single dose.

855. The method of any one of embodiments 1-846, wherein no more than about 25000 μg of the fusion protein is administered to the subject in a single dose.

856. The method of any one of embodiments 1-846, wherein no more than about 23000 μg of the fusion protein is administered to the subject in a single dose.

857. The method of any one of embodiments 1-846, wherein no more than about 20000 μg of the fusion protein is administered to the subject in a single dose.

858. The method of any one of embodiments 1-846, wherein no more than about 15000 μg of the fusion protein is administered to the subject in a single dose.

859. The method of any one of embodiments 1-846, wherein no more than about 10000 μg of the fusion protein is administered to the subject in a single dose.

860. The method of any preceding embodiment, wherein at least about 225 μg, 250 μg, 275 μg, 300 μg, 400 μg, 500 μg, 600 μg, 625 μg, 650 μg, 675 μg, 700 μg, 800 μg, 900 μg, 1000 μg, 1100 μg, 1200 μg, 1300 μg, 1400 μg, 1500 μg, 1600 μg, 1700 μg, 1800 μg, 1900 μg, 2000 μg, 2100 μg, 2200 μg, 2300 μg, 2400 μg, 2500 μg, 2600 μg, 2700 μg, 2800 μg, 2900 μg, or 3000 μg of the fusion protein is administered to the subject in a single dose.

861. The method of any one of embodiments 1-846 and 854-859, wherein at least about 800 μg of the fusion protein is administered to the subject in a single dose.

862. The method of any one of embodiments 1-846 and 854-859, wherein at least about 1000 μg of the fusion protein is administered to the subject in a single dose.

863. The method of any one of embodiments 1-846 and 854-859, wherein at least about 1500 μg of the fusion protein is administered to the subject in a single dose.

864. The method of any one of embodiments 1-846 and 854-859, wherein at least about 2000 μg of the fusion protein is administered to the subject in a single dose.

865. The method of any one of embodiments 1-846 and 854-859, wherein at least about 2500 μg of the fusion protein is administered to the subject in a single dose.

866. The method of any one of embodiments 1-846 and 854-859, wherein at least about 3000 μg of the fusion protein is administered to the subject in a single dose.

867. The method of any preceding embodiment, wherein at least two doses of the fusion protein are administered at least 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days apart.

868. The method of embodiment 867, wherein at least two doses of the fusion protein are administered at least 10 days apart.

869. The method of embodiment 867 or 868, wherein at least two doses of the fusion protein are administered at least 14 days apart.

870. The method of any one of embodiments 867-869, wherein at least two doses of the fusion protein are administered at least 21 days apart.

871. The method of embodiment 867-870, wherein at least two doses of the fusion protein are administered at least 28 days apart.

872. The method of embodiment 867, wherein (i) at least two doses of the fusion protein are administered at least 10 days apart; and (ii) at least two additional doses of the fusion protein are administered at least 21 days apart.

873. The method of embodiment 867, wherein (i) at least two doses of the fusion protein are administered at least 14 days apart; and (ii) at least two additional doses of the fusion protein are administered at least 21 days apart. 874. The method of embodiment 867, wherein (i) at least two doses of the fusion protein are administered at least 14 days apart; and (ii) at least two additional doses of the fusion protein are administered at least 28 days apart.

875. The method of any one of embodiments 1-867, wherein at least two doses of the fusion protein are administered at least 1, 2, 3, 4, 5, 6, 7, 8, 9, 10, 11, 12, 13, 14, 15, 16, 17, 18, 19, or 20 weeks apart.

876. The method of embodiment 875, wherein at least two doses of the fusion protein are administered at least 1 week apart.

877. The method of embodiment 875, wherein at least two doses of the fusion protein are administered at least 2 weeks apart.

878. The method of embodiment 875, wherein at least two doses of the fusion protein are administered at least 3 weeks apart.

879. The method of embodiment 875, wherein at least two doses of the fusion protein are administered at least 4 weeks apart.

880. The method of embodiment 875, wherein (i) at least two doses of the fusion protein are administered at least 1 week apart; and (ii) at least two additional doses of the fusion protein are administered at least 3 weeks apart.

881. The method of embodiment 875, wherein (i) at least two doses of the fusion protein are administered at least 2 weeks apart; and (ii) at least two additional doses of the fusion protein are administered at least 3 weeks apart.

882. The method of embodiment 875, wherein (i) at least two doses of the fusion protein are administered at least 2 weeks apart; and (ii) at least two additional doses of the fusion protein are administered at least 4 weeks apart.

883. The method of any preceding embodiment, wherein the method further comprises pausing administration of the fusion protein for at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 weeks.

884. The method of embodiment 883, wherein administration of the fusion protein is paused for at least about 8 weeks.

885. The method of embodiment 883, wherein administration of the fusion protein is paused for at least about 10 weeks.

886. The method of embodiment 883, wherein administration of the fusion protein is paused for at least about 12 weeks.

887. The method of embodiment 883, wherein administration of the fusion protein is paused for at least about 14 weeks.

888. The method of embodiment 883, wherein administration of the fusion protein is paused for at least about 16 weeks.

889. The method of embodiment 883, wherein administration of the fusion protein is paused for at least about 18 weeks.

890. The method of embodiment 883, wherein administration of the fusion protein is paused for at least about 20 weeks.

891. The method of any preceding embodiment, wherein the method further comprises pausing administration of the fusion protein for at least about 1, 2, 3, 4, 5, 6, 7, 8, 9, 10, 11, or 12 months.

892. The method of embodiment 891, wherein administration of the fusion protein is paused for at least about 2 months.

893. The method of embodiment 891, wherein administration of the fusion protein is paused for at least about 3 months.

894. The method of embodiment 891, wherein administration of the fusion protein is paused for at least about 4 months.

895. The method of embodiment 891, wherein administration of the fusion protein is paused for at least about 5 months.

896. The method of embodiment 891, wherein administration of the fusion protein is paused for at least about 6 months.

897. The method of any one of embodiments 883-896, wherein at least about 2, 3, 4, 5, 6, 7, 8, 9, 10, 11, 12, 13, 14, 15, 16, 17, 18, 19, or 20 doses of the fusion protein are administered to the subject prior to pausing administration of the fusion protein.

898. The method of embodiment 897, wherein at least about 3 doses of the fusion protein are administered to the subject prior to pausing administration of the fusion protein.

899. The method of embodiment 897, wherein at least about 4 doses of the fusion protein are administered to the subject prior to pausing administration of the fusion protein.

900. The method of embodiment 897, wherein at least about 5 doses of the fusion protein are administered to the subject prior to pausing administration of the fusion protein.

901. The method of embodiment 897, wherein at least about 6 doses of the fusion protein are administered to the subject prior to pausing administration of the fusion protein.

902. The method of embodiment 897, wherein at least about 7 doses of the fusion protein are administered to the subject prior to pausing administration of the fusion protein.

903. The method of embodiment 897, wherein at least about 8 doses of the fusion protein are administered to the subject prior to pausing administration of the fusion protein.

904. The method of any one of embodiments 883-903, wherein less than about 20, 19, 18, 17, 16, 15, 14, 13, 12, 11, 10, or 9 doses of the fusion protein are administered to the subject prior to pausing administration of the fusion protein.

905. The method of embodiment 904, wherein less than about 12 doses of the fusion protein are administered to the subject prior to pausing administration of the fusion protein.

906. The method of embodiment 904, wherein less than about 10 doses of the fusion protein are administered to the subject prior to pausing administration of the fusion protein.

907. The method of embodiment 904, wherein less than about 8 doses of the fusion protein are administered to the subject prior to pausing administration of the fusion protein.

908. The method of any one of embodiments 883-907, wherein about 2 to about 15, about 2 to about 12, about 2 to about 10, about 2 to about 8, about 3 to about 15, about 3 to about 12, about 3 to about 10, about 3 to about 8, about 4 to about 15, about 4 to about 12, about 4 to about 10, about 4 to about 8, about 5 to about 15, about 5 to about 12, about 5 to about 10, about 5 to about 8, about 6 to about 15, about 6 to about 12, about 6 to about 10, about 6 to about 8 doses of the fusion protein are administered to the subject prior to pausing administration of the fusion protein.

909. The method of embodiment 908, wherein about 2 to about 10 doses of the fusion protein are administered to the subject prior to pausing administration of the fusion protein.

910. The method of embodiment 908, wherein about 3 to about 12 doses of the fusion protein are administered to the subject prior to pausing administration of the fusion protein.

911. The method of embodiment 908, wherein about 3 to about 9 doses of the fusion protein are administered to the subject prior to pausing administration of the fusion protein.

912. The method of embodiment 908, wherein about 4 to about 12 doses of the fusion protein are administered to the subject prior to pausing administration of the fusion protein.

913. The method of embodiment 908, wherein about 4 to about 9 doses of the fusion protein are administered to the subject prior to pausing administration of the fusion protein.

914. The method of any preceding embodiment, wherein a plurality of doses of the fusion protein is administered over a duration of at least about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks.

915. The method of embodiment 914, wherein the plurality of doses of the fusion protein is administered over a duration of at least about 6 weeks.

916. The method of embodiment 914, wherein the plurality of doses of the fusion protein is administered over a duration of at least about 8 weeks.

917. The method of embodiment 914, wherein the plurality of doses of the fusion protein is administered over a duration of at least about 10 weeks.

918. The method of embodiment 914, wherein the plurality of doses of the fusion protein is administered over a duration of at least about 14 weeks.

919. The method of embodiment 914, wherein the plurality of doses of the fusion protein is administered over a duration of at least about 18 weeks.

920. The method of embodiment 914, wherein the plurality of doses of the fusion protein is administered over a duration of at least about 20 weeks.

921. The method of embodiment 914, wherein the plurality of doses of the fusion protein is administered over a duration of at least about 30 weeks.

922. The method of any preceding embodiment, wherein a plurality of doses of the fusion protein is administered over a duration of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 months.

923. The method of embodiment 922, wherein the plurality of doses of the fusion protein is administered over a duration of at least about 2 months.

924. The method of embodiment 922, wherein the plurality of doses of the fusion protein is administered over a duration of at least about 3 months.

925. The method of embodiment 922, wherein the plurality of doses of the fusion protein is administered over a duration of at least about 4 months.

926. The method of embodiment 922, wherein the plurality of doses of the fusion protein is administered over a duration of at least about 6 months.

927. The method of embodiment 922, wherein the plurality of doses of the fusion protein is administered over a duration of at least about 10 months.

928. The method of embodiment 922, wherein the plurality of doses of the fusion protein is administered over a duration of at least about 12 months.

929. The method of embodiment 922, wherein the plurality of doses of the fusion protein is administered over a duration of at least about 14 months.

930. The method of any preceding embodiment, wherein the fusion protein is administered to the subject intravenously, intratumorally, subcutaneously, intradermally, intramuscularly, intraperitoneally, intravesically, intracranially, intrathecally, intracavitary or intraventricularly.

931. The method of any one of embodiments 1-929, wherein the fusion protein is administered to the subject intravenously.

932. The method of any one of embodiments 1-929, wherein the fusion protein is administered to the subject subcutaneously.

1.-127. (canceled)

128. A method of preventing, reducing and/or inhibiting the recurrence, growth, proliferation, migration and/or metastasis of a cancer cell or population of cancer cells in a subject in need thereof or treating and/or inhibiting cancer in a subject in need thereof or enhancing, promoting, and/or increasing the tumor infiltration of T-cells and/or NK cells in a subject in need thereof or enhancing, promoting, and/or accelerating the recovery from or reversing the effects of lymphopenia in a subject in need thereof or promoting, inducing and/or increasing the expansion and/or proliferation of a cell or a population of cells that express fms related tyrosine kinase 3 (FLT3, CD135) in a subject in need thereof or inducing the immune system in a subject in need thereof, comprising administering to the subject (I) an effective amount of a human fms related tyrosine kinase 3 ligand (FLT3L) modulator; and (II) an effective amount of sacituzumab govitecan.

129. (canceled)

130. (canceled)

131. (canceled)

132. (canceled)

133. (canceled)

134. A method of preventing, reducing and/or inhibiting the recurrence, growth, proliferation, migration and/or metastasis of a cancer cell or population of cancer cells in a subject in need thereof or treating and/or inhibiting cancer in a subject in need thereof or enhancing, promoting, and/or increasing the tumor infiltration of T-cells and/or NK cells in a subject in need thereof or enhancing, promoting, and/or accelerating the recovery from or reversing the effects of lymphopenia in a subject in need thereof or promoting, inducing and/or increasing the expansion and/or proliferation of a cell or a population of cells that express fms related tyrosine kinase 3 (FLT3, CD135) in a subject in need thereof or inducing the immune system in a subject in need thereof, comprising administering to the subject (I) an effective amount human fms related tyrosine kinase 3 ligand (FLT3L) modulator; and (II) an effective amount of one or more therapeutic agents selected from the group consisting of an immunoconjugate, FLT3R agonist, anti-PD1 antibody, anti-PDL1 antibody, anti-Tigit antibody, anti-TREM1/2 antibody, anti-CCR8 antibody, MCL-1 inhibitor, anti-CD47 antibody, adenosine pathway inhibitor.

135. (canceled)

136. (canceled)

137. (canceled)

138. (canceled)

139. (canceled)

140. The method of claim 128, wherein the FLT3L modulator is a fusion protein comprising a FLT3L protein or fragment thereof and an Fc protein or fragment thereof.

141. The method of claim 140, wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18, 21-27, 114, and 115.

142. The method of claim 140, wherein the Fc protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 111.

143. The method of claim 142, wherein residues 13-17 of SEQ ID NO: 111 comprise the amino acid sequence PVAGT (SEQ ID NO: 116) and residue 76 of SEQ ID NO: 111 is a glycine.

144. The method of claim 140, wherein the FLT3L protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NOs: 112, 113, or 117.

145. The method of claim 140, wherein the FLT3L protein or fragment thereof comprises CDX-301.

146. The method of claim 134, wherein the immunoconjugate is co-administered with the FLT3L modulator.

147. The method of claim 128, wherein the FLT3L modulator comprises the amino acid sequence of any one of SEQ ID NOs: 101-105 and 107.

148. The method of claim 134, wherein the immunoconjugate comprises datopotamab deruxtecan (DS-1062).

149. The method of claim 134, wherein the FLT3L modulator is a fusion protein comprising a FLT3L protein or fragment thereof and an Fc protein or fragment thereof.

150. The method of claim 149, wherein the fusion protein comprises an amino acid sequence that is at least 80%, at least 85%, at least 90%, at least 91%, at least 92%, at least 93%, at least 94%, at least 95%, at least 96%, at least 97%, at least 98%, or at least 99% identical to an amino acid sequence selected from the group consisting of SEQ ID NOs: 1-18, 21-27, 114, and 115.

151. The method of claim 149, wherein the Fc protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NO: 111.

152. The method of claim 151, wherein residues 13-17 of SEQ ID NO: 111 comprise the amino acid sequence PVAGT (SEQ ID NO: 116) and residue 76 of SEQ ID NO: 111 is a glycine.

153. The method of claim 149, wherein the FLT3L protein or fragment thereof comprises an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence of SEQ ID NOs: 112, 113, or 117.

154. The method of claim 149, wherein the FLT3L protein or fragment thereof comprises CDX-301.

155. The method of claim 134, wherein the FLT3L modulator comprises the amino acid sequence of any one of SEQ ID NOs: 101-105 and 107.


SEQUENCE LISTING (117 sequences)

SEQ ID NO: 1 - protein, 369 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 364 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 386 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 386 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 369 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNIARLLQETSEQLVALKPWITRQNFARCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 381 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 359 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 376 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 369 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 364 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 386 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 386 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 369 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNIARLLQETSEQLVALKPWITRQNFARCLELQCQPDSSTLPPPWSPRPLEATAPTAPQGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 381 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 359 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 376 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 376 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNIARLLQETSEQLVALKPWITRQNFARCLELQCQPDSSTLPPPWSPRPESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 376 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNIARLLQETSEQLVALKPWITRQNFARCLELQCQPDSSTLPPPWSPRPESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 389 aa, Artificial Sequence
  Description of Artificial Sequence Synthetic polypeptide
TPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 20 - protein, 389 aa, Artificial Sequence
  Description of Artificial Sequence Synthetic polypeptide
TPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTSTQLLALKPCIGKACQNFSRCLEVQCQPDSSTLLPPRSPIALEATELPEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 21 - protein, 369 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 22 - protein, 369 aa, Artificial Sequence
  Description of Artificial Sequence Synthetic polypeptide
TQDCSFQYSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 369 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTECAFQPPPSCLRFVQTNISRLLQETSEQLVALKPWITRQNFSRCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 369 aa, Artificial Sequence
  Description of Artificial Sequence Synthetic polypeptide
TQDCSFQYSPISSDFAVKIRELSDYLLQDYPVTVASNLQDEELCGGLWRLVLAQRWMERLKTVAGSKMQGLLERVNTEIHFVTECAFQPPPSCLRFVQTNISRLLQETSEQLVALKPWITRQNFSRCLELQCQPDSSTLPPPWSPRPLEATAPTAPQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 381 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNIARLLQETSEQLVALKPWITRQNFARCLELQCQPDSSTLPPPWSPRPLEATAESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 381 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDAAALPPPWSPRPLEATAESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 381 aa, Artificial Sequence
  Description of Artificial Sequence Synthetic polypeptide
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDAAALPPPWAPRPLEATAEAKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - DNA, 1107 bp, Artificial Sequence
  Description of Artificial Sequence Synthetic polynucleotide
acacaggattgcagcttccagcacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccaagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctctggaagccacagctcccaccgctcctcaaggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 29 - DNA, 1107 bp, Artificial Sequence
  Description of Artificial Sequence Synthetic polynucleotide
acccaggactgctccttccagcactcccctatctcttccgacttcgccgtgaagatcagagagctgtccgactacctgctgcaggactaccctgtgaccgtggccagcaatctgcaggacgaagaactgtgtggtggcctgtggcgactggtgttggctcagagatggatggaacggctgaaaaccgtggccggctctaagatgcagggcctgctggaaagagtgaacaccgagatccacttcgtgaccaagtgcgcctttcagcctcctccatcctgcctgagattcgtgcagaccaacatctcccggctgctgcaagagacatccgagcagctggtggctctgaagccctggatcacccggcagaacttctctcggtgtctggaactgcagtgtcagcccgactcttctaccctgcctccaccttggagccccagacctttggaagctaccgctccaacagctcctcaaggcggcccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgccacctagccgggaagagatgaccaagaaccaggtgtccctgacctgcctggtcaagggcttctacccctctgatatcgccgtggaatgggagagcaatggccagcctgagaacaactacaagaccacacctcctgtgctggactccgacggctcattcttcctgtactccaagctgacagtggacaagtccagatggcagcagggcaacgtgttctcctgctccgtgatgcacgaggctctgcacaaccactacacccagaagtccctgtctctgtcccctggcaaa

SEQ ID NO: 30 - DNA, 1092 bp, Artificial Sequence
  Description of Artificial Sequence Synthetic polynucleotide
acacaggattgcagcttccagcacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccaagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctctggaagccacagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 31 - DNA, 1092 bp, Artificial Sequence
  Description of Artificial Sequence Synthetic polynucleotide
acccaggactgctccttccagcactcccctatctcttccgacttcgccgtgaagatcagagagctgtccgactacctgctgcaggactatcctgtgaccgtggccagcaacctgcaggatgaagaactgtgtggtggactgtggcgactggtgctggctcagagatggatggaaagactgaaaaccgtggccggctccaagatgcagggactgctggaaagagtgaacacagagatccacttcgtgaccaagtgcgcctttcagcctcctccatcctgcctgagattcgtgcagaccaacatctcccggctgctgcaagagacatctgagcagctggtggccctgaagccttggatcacccggcagaacttctctcggtgcctggaactgcagtgtcagcctgattcttctaccctgcctccaccttggagccctagacctttggaagctacagctggcggcccaagcgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaacctcaggtttacaccctgccacctagccgggaagagatgaccaaaaaccaggtgtccctgacctgcctggtcaagggcttctacccatccgatatcgccgtggaatgggagtctaacggccagcctgagaacaactacaagaccacacctcctgtgctggactccgacggctcattcttcctgtactccaagctgacagtggacaagtctcggtggcagcagggcaacgtgttctcctgttctgtgatgcacgaggccctgcacaaccactacacccagaagtccctgtctctgtcccctggcaaa

SEQ ID NO: 32 - DNA, 1158 bp, Artificial Sequence
  Description of Artificial Sequence Synthetic polynucleotide
acacaggattgcagcttccagcacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccaagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctctggaagccacagctcccaccgctcctcaagaatctaagtacggccctccctgccctccttgcccagcccctgaatttgagggcggaccctccgtgttcctgttccccccaaagcccaaggacaccctgatgatcagccggacccccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgtccctgggcaaa

SEQ ID NO: 33 - DNA, 1158 bp, Artificial Sequence
  Description of Artificial Sequence Synthetic polynucleotide
acccaggactgctccttccagcactcccctatctcttccgacttcgccgtgaagatcagagagctgtccgactacctgctgcaggactatcctgtgaccgtggccagcaacctgcaggatgaagaactgtgtggtggactgtggcgactggtgctggctcagagatggatggaaagactgaaaaccgtggccggctccaagatgcagggactgctggaaagagtgaacacagagatccacttcgtgaccaagtgcgcctttcagcctcctccatcctgcctgagattcgtgcagaccaacatctcccggctgctgcaagagacatctgagcagctggtggccctgaagccttggatcacccggcagaacttctctcggtgcctggaactgcagtgtcagcctgattcttctaccctgcctccaccttggagccctagacctttggaggctacagctcctaccgctcctcaagagtctaagtacggccctccttgtcctccatgtcctgctccagaatttgaaggcggcccaagcgtgttcctgtttcctccaaagcctaaggacaccctgatgatctctcggacccctgaagtgacctgcgtggtggtggatgtgtctcaagaggaccccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgcctagctccatcgaaaagaccatctccaaggccaagggccagccaagagaacctcaggtgtacacactgcctccaagccaagaggaaatgaccaagaaccaggtgtccctgacctgcctggtcaagggcttctacccatccgatatcgccgtggaatgggagtctaacggccagcctgagaacaactacaagaccacacctcctgtgctggactccgacggctccttctttctgtactctcgcctgaccgtggacaagtctagatggcaagagggcaacgtgttctcctgctctgtgatgcacgaggccctgcacaaccactacacccagaagtccctgtctctgtccctgggcaaa

SEQ ID NO: 34 - DNA, 1158 bp, Artificial Sequence
  Description of Artificial Sequence Synthetic polynucleotide
acacaggattgcagcttccagcacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccaagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctctggaagccacagctcccaccgctcctcaagagtctaagtacggccctccttgtcctccatgtcctgctccagaagctgctggcggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggccagcctagggaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggcaaa

SEQ ID NO: 35 - DNA, 1158 bp, Artificial Sequence
  Description of Artificial Sequence Synthetic polynucleotide
acccaggactgctccttccagcactcccctatctcttccgacttcgccgtgaagatcagagagctgtccgactacctgctgcaggactatcctgtgaccgtggccagcaacctgcaggatgaagaactgtgtggtggactgtggcgactggtgctggctcagagatggatggaaagactgaaaaccgtggccggctccaagatgcagggactgctggaaagagtgaacacagagatccacttcgtgaccaagtgcgcctttcagcctcctccatcctgcctgagattcgtgcagaccaacatctcccggctgctgcaagagacatctgagcagctggtggccctgaagccttggatcacccggcagaacttctctcggtgcctggaactgcagtgtcagcctgattcttctaccctgcctccaccttggagccctagacctttggaggctacagctcctaccgctcctcaagagtctaagtacggccctccttgtcctccatgtcctgctccagaagctgctggcggaccaagcgttttcctgtttcctccaaagcctaaggacaccctgatgatctctcggacccctgaagtgacctgcgtggtggtggatgtgtctcaagaggaccccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgcctagctccatcgaaaagaccatctccaaggccaagggccagccaagagaacctcaggtgtacacactgcctccaagccaagaggaaatgaccaagaaccaggtgtccctgacctgcctggtcaagggcttctacccatccgatatcgccgtggaatgggagtctaacggccagcctgagaacaactacaagaccacacctcctgtgctggactccgacggctccttctttctgtactctcgcctgaccgtggacaagtctagatggcaagagggcaacgtgttctcctgctctgtgatgcacgaggccctgcacaaccactacacccagaagtccctgtctctgtccctgggcaaa

SEQ ID NO: 36 - DNA, 1185 bp, Artificial Sequence
  Description of Artificial Sequence Synthetic polynucleotide
atgacagttttggctccagcttggtcccctacaacctacctgctgctgctgttgctgctctcctctggcctgtctggcacccaggactgttccttccagcactcccctatctccagcgacttcgccgtgaagatcagagagctgtccgactatctgctgcaggactaccctgtgaccgtggccagcaatctgcaggacgaagaactgtgtggtggcctgtggcgactggtgttggctcagagatggatggaacggctgaaaaccgtggccggctctaagatgcagggcctgctggaaagagtgaacaccgagatccacttcgtgaccaagtgcgcctttcagcctcctccatcctgcctgagattcgtgcagaccaatatcgcccggctgctgcaagagacatccgagcagctggtggctctgaagccctggatcaccagacagaacttcgcccggtgtctggaactgcagtgtcagcctgacagctctaccctgcctccaccttggagccctagacctctggaagctaccgctccaaccgctcctcaagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 37 - DNA, 1107 bp, Artificial Sequence
  Description of Artificial Sequence Synthetic polynucleotide
acccaggactgctccttccagcactcccctatctcttccgacttcgccgtgaagatcagagagctgtccgactacctgctgcaggactatcctgtgaccgtggccagcaacctgcaggatgaagaactgtgtggtggactgtggcgactggtgctggctcagagatggatggaaagactgaaaaccgtggccggctccaagatgcagggactgctggaaagagtgaacacagagatccacttcgtgaccaagtgcgcctttcagcctcctccatcctgcctgagattcgtgcagaccaatatcgcccggctgctgcaagagacatctgagcagctggtggccctgaagccttggatcaccagacagaacttcgcccggtgcctggaactgcagtgtcagcctgattcttctaccctgcctccaccttggagccctagacctttggaggctacagctcctactgctcctcaaggcggcccaagcgttttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaacctcaggtttacaccctgccacctagccgggaagagatgaccaaaaaccaggtgtccctgacctgcctggtcaagggcttctacccatccgatatcgccgtggaatgggagtctaacggccagcctgagaacaactacaagaccacacctcctgtgctggactccgacggctcattcttcctgtactccaagctgacagtggacaagtctcggtggcagcagggcaacgtgttctcctgttctgtgatgcacgaggccctgcacaaccactacacccagaagtccctgtctctgtcccctggcaaa

SEQ ID NO: 38 - DNA, 1143 bp, Artificial Sequence
  Description of Artificial Sequence Synthetic polynucleotide
acacaggattgcagcttccagcacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccaagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctctggaagccacagctgagtctaagtacggccctccttgtcctccatgtcctgctccagaagctgctggcggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggccagcctagggaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggcaaa

SEQ ID NO: 39 - DNA, 1143 bp, Artificial Sequence
  Description of Artificial Sequence Synthetic polynucleotide
acccaggactgctccttccagcactcccctatctcttccgacttcgccgtgaagatcagagagctgtccgactacctgctgcaggactatcctgtgaccgtggccagcaacctgcaggatgaagaactgtgtggtggactgtggcgactggtgctggctcagagatggatggaaagactgaaaaccgtggccggctccaagatgcagggactgctggaaagagtgaacacagagatccacttcgtgaccaagtgcgcctttcagcctcctccatcctgcctgagattcgtgcagaccaacatctcccggctgctgcaagagacatctgagcagctggtggccctgaagccttggatcacccggcagaacttctctcggtgcctggaactgcagtgtcagcctgattcttctaccctgcctccaccttggagccctagacctctggaagctaccgccgagtctaagtacggacctccttgtcctccatgtcctgctccagaagctgctggcggaccaagcgttttcctgtttcctccaaagcctaaggacaccctgatgatctctcggacccctgaagtgacctgcgtggtggtggatgtgtctcaagaggaccccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgcctagctccatcgaaaagaccatctccaaggccaagggccagccaagagaacctcaggtgtacacactgcctccaagccaagaggaaatgaccaagaaccaggtgtccctgacctgcctggtcaagggcttctacccatccgatatcgccgtggaatgggagtctaacggccagcctgagaacaactacaagaccacacctcctgtgctggactccgacggctccttctttctgtactctcgcctgaccgtggacaagtctagatggcaagagggcaacgtgttctcctgctctgtgatgcacgaggccctgcacaaccactacacccagaagtccctgtctctgtccctgggcaaa

SEQ ID NO: 40 - DNA, 1077 bp, Artificial Sequence
  Description of Artificial Sequence Synthetic polynucleotide
acacaggattgcagcttccagcacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccaagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 41 - DNA, 1077 bp, Artificial Sequence
  Description of Artificial Sequence Synthetic polynucleotide
acccaggactgctccttccagcactcccctatctcttccgacttcgccgtgaagatcagagagctgtccgactacctgctgcaggactatcctgtgaccgtggccagcaacctgcaggatgaagaactgtgtggtggactgtggcgactggtgctggctcagagatggatggaaagactgaaaaccgtggccggctccaagatgcagggactgctggaaagagtgaacacagagatccacttcgtgaccaagtgcgcctttcagcctcctccatcctgcctgagattcgtgcagaccaacatctcccggctgctgcaagagacatctgagcagctggtggccctgaagccttggatcacccggcagaacttctctcggtgcctggaactgcagtgtcagcctgattcttctaccctgcctccaccttggagtcctagacctggcggaccaagcgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaacctcaggtttacaccctgccacctagccgggaagagatgaccaaaaaccaggtgtccctgacctgcctggtcaagggcttctacccatccgatatcgccgtggaatgggagtctaacggccagcctgagaacaactacaagaccacacctcctgtgctggactccgacggctcattcttcctgtactccaagctgacagtggacaagtctcggtggcagcagggcaacgtgttctcctgttctgtgatgcacgaggccctgcacaaccactacacccagaagtccctgtctctgtcccctggcaaa

SEQ ID NO: 42 - DNA, 1128 bp, Artificial Sequence
  Description of Artificial Sequence Synthetic polynucleotide
acacaggattgcagcttccagcacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccaagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctgagtctaagtacggccctccttgtcctccatgtcctgctccagaagctgctggcggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggccagcctagggaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggcaaa

SEQ ID NO: 43 - DNA, 1128 bp, Artificial Sequence
  Description of Artificial Sequence Synthetic polynucleotide
acccaggactgctccttccagcactcccctatctcttccgacttcgccgtgaagatcagagagctgtccgactacctgctgcaggactatcctgtgaccgtggccagcaacctgcaggatgaagaactgtgtggtggactgtggcgactggtgctggctcagagatggatggaaagactgaaaaccgtggccggctccaagatgcagggactgctggaaagagtgaacacagagatccacttcgtgaccaagtgcgcctttcagcctcctccatcctgcctgagattcgtgcagaccaacatctcccggctgctgcaagagacatctgagcagctggtggccctgaagccttggatcacccggcagaacttctctcggtgcctggaactgcagtgtcagcctgattcttctaccctgcctccaccttggagccctcggcctgaatctaagtatggccctccttgtcctccatgtcctgctccagaagctgctggcggaccaagcgttttcctgtttcctccaaagcctaaggacaccctgatgatctctcggacccctgaagtgacctgcgtggtggtggatgtgtctcaagaggaccccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgcctagctccatcgaaaagaccatctccaaggccaagggccagccaagagaacctcaggtgtacacactgcctccaagccaagaggaaatgaccaagaaccaggtgtccctgacctgcctggtcaagggcttctacccatccgatatcgccgtggaatgggagtctaacggccagcctgagaacaactacaagaccacacctcctgtgctggactccgacggctccttctttctgtactctcgcctgaccgtggacaagtctagatggcaagagggcaacgtgttctcctgctctgtgatgcacgaggccctgcacaaccactacacccagaagtccctgtctctgtccctgggcaaa

SEQ ID NO: 44 - DNA, 1107 bp, Artificial Sequence
  Description of Artificial Sequence Synthetic polynucleotide
acacaggattgcagcttccagcacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccaagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctctggaagccacagctcccaccgctcctcaaggcggaccgtcagtcttcctcttccccccaaaacccaaggacaccctctacatcacccgggaacctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 45 - DNA, 1107 bp, Artificial Sequence
  Description of Artificial Sequence Synthetic polynucleotide
acccaggactgctccttccagcactcccctatctcttccgacttcgccgtgaagatcagagagctgtccgactacctgctgcaggactatcctgtgaccgtggccagcaacctgcaggatgaagaactgtgtggtggactgtggcgactggtgctggctcagagatggatggaaagactgaaaaccgtggccggctccaagatgcagggactgctggaaagagtgaacacagagatccacttcgtgaccaagtgcgcctttcagcctcctccatcctgcctgagattcgtgcagaccaacatctcccggctgctgcaagagacatctgagcagctggtggccctgaagccttggatcacccggcagaacttctctcggtgcctggaactgcagtgtcagcctgattcttctaccctgcctccaccttggagccctagacctttggaggctacagctcctactgctcctcaaggcggcccaagcgttttcctgtttcctccaaagcctaaggacaccctgtacatcacccgcgagcctgaagtgacatgcgtggtggtggatgtgtcccacgaggaccccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaacctcaggtttacaccctgccacctagccgggaagagatgaccaaaaaccaggtgtccctgacctgcctggtcaagggcttctacccatccgatatcgccgtggaatgggagtctaacggccagcctgagaacaactacaagaccacacctcctgtgctggactccgacggctcattcttcctgtactccaagctgacagtggacaagtctcggtggcagcagggcaacgtgttctcctgttctgtgatgcacgaggccctgcacaaccactacacccagaagtccctgtctctgtcccctggcaaa

SEQ ID NO: 46 - DNA, 1092 bp, Artificial Sequence
  Description of Artificial Sequence Synthetic polynucleotide
acacaggattgcagcttccagcacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccaagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctctggaagccacagctgggggaccgtcagtcttcctcttccccccaaaacccaaggacaccctctacatcacccgggaacctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 47 - DNA, 1092 bp, Artificial Sequence
  Description of Artificial Sequence Synthetic polynucleotide
acccaggactgctccttccagcactcccctatctcttccgacttcgccgtgaagatcagagagctgtccgactacctgctgcaggactatcctgtgaccgtggccagcaacctgcaggatgaagaactgtgtggtggactgtggcgactggtgctggctcagagatggatggaaagactgaaaaccgtggccggctccaagatgcagggactgctggaaagagtgaacacagagatccacttcgtgaccaagtgcgcctttcagcctcctccatcctgcctgagattcgtgcagaccaacatctcccggctgctgcaagagacatctgagcagctggtggccctgaagccttggatcacccggcagaacttctctcggtgcctggaactgcagtgtcagcctgattcttctaccctgcctccaccttggagccctagacctttggaagctacagctggcggcccaagcgtgttcctgtttcctccaaagcctaaggacaccctgtacatcacccgcgagcctgaagtgacatgcgtggtggtggatgtgtcccacgaggaccccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaacctcaggtttacaccctgccacctagccgggaagagatgaccaaaaaccaggtgtccctgacctgcctggtcaagggcttctacccatccgatatcgccgtggaatgggagtctaacggccagcctgagaacaactacaagaccacacctcctgtgctggactccgacggctcattcttcctgtactccaagctgacagtggacaagtctcggtggcagcagggcaacgtgttctcctgttctgtgatgcacgaggccctgcacaaccactacacccagaagtccctgtctctgtcccctggcaaa

SEQ ID NO: 48 - DNA, 1158 bp, Artificial Sequence
  Description of Artificial Sequence Synthetic polynucleotide
acacaggattgcagcttccagcacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccaagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctctggaagccacagctcccaccgctcctcaagaatctaagtacggccctccctgccctccttgcccagcccctgaatttgagggcggaccctccgtgttcctgttccccccaaagcccaaggacaccctgtacatcacccgggaacccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgtccctgggcaaa

SEQ ID NO: 49 - DNA, 1158 bp, Artificial Sequence
  Description of Artificial Sequence Synthetic polynucleotide
acccaggactgctccttccagcactcccctatctcttccgacttcgccgtgaagatcagagagctgtccgactacctgctgcaggactatcctgtgaccgtggccagcaacctgcaggatgaagaactgtgtggtggactgtggcgactggtgctggctcagagatggatggaaagactgaaaaccgtggccggctccaagatgcagggactgctggaaagagtgaacacagagatccacttcgtgaccaagtgcgcctttcagcctcctccatcctgcctgagattcgtgcagaccaacatctcccggctgctgcaagagacatctgagcagctggtggccctgaagccttggatcacccggcagaacttctctcggtgcctggaactgcagtgtcagcctgattcttctaccctgcctccaccttggagccctagacctttggaggctacagctcctaccgctcctcaagagtctaagtacggccctccttgtcctccatgtcctgctccagaatttgaaggcggcccaagcgtgttcctgtttcctccaaagcctaaggacaccctgtacatcacccgcgagcctgaagtgacatgcgtggtggtggatgtgtcccaagaggaccccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgcctagctccatcgaaaagaccatctccaaggccaagggccagccaagagaacctcaggtgtacacactgcctccaagccaagaggaaatgaccaagaaccaggtgtccctgacctgcctggtcaagggcttctacccatccgatatcgccgtggaatgggagtctaacggccagcctgagaacaactacaagaccacacctcctgtgctggactccgacggctccttctttctgtactctcgcctgaccgtggacaagtctagatggcaagagggcaacgtgttctcctgctctgtgatgcacgaggccctgcacaaccactacacccagaagtccctgtctctgtccctgggcaaa

SEQ ID NO: 50 - DNA, 1158 bp, Artificial Sequence
  Description of Artificial Sequence Synthetic polynucleotide
acacaggattgcagcttccagcacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccaagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctctggaagccacagctcccaccgctcctcaagagtctaagtacggccctccttgtcctccatgtcctgctccagaagctgctggcggcccttccgtgtttctgttccctccaaagcctaaggacaccctgtacatcacccgggaacccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgtccctgggcaaa

SEQ ID NO: 51 - DNA, 1158 bp, Artificial Sequence
  Description of Artificial Sequence Synthetic polynucleotide
acccaggactgctccttccagcactcccctatctcttccgacttcgccgtgaagatcagagagctgtccgactacctgctgcaggactatcctgtgaccgtggccagcaacctgcaggatgaagaactgtgtggtggactgtggcgactggtgctggctcagagatggatggaaagactgaaaaccgtggccggctccaagatgcagggactgctggaaagagtgaacacagagatccacttcgtgaccaagtgcgcctttcagcctcctccatcctgcctgagattcgtgcagaccaacatctcccggctgctgcaagagacatctgagcagctggtggccctgaagccttggatcacccggcagaacttctctcggtgcctggaactgcagtgtcagcctgattcttctaccctgcctccaccttggagccctagacctttggaggctacagctcctaccgctcctcaagagtctaagtacggccctccttgtcctccatgtcctgctccagaagctgctggcggaccaagcgttttcctgtttcctccaaagcctaaggacaccctgtacatcacccgcgagcctgaagtgacatgcgtggtggtggatgtgtcccaagaggaccccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgcctagctccatcgaaaagaccatctccaaggccaagggccagccaagagaacctcaggtgtacacactgcctccaagccaagaggaaatgaccaagaaccaggtgtccctgacctgcctggtcaagggcttctacccatccgatatcgccgtggaatgggagtctaacggccagcctgagaacaactacaagaccacacctcctgtgctggactccgacggctccttctttctgtactctcgcctgaccgtggacaagtctagatggcaagagggcaacgtgttctcctgctctgtgatgcacgaggccctgcacaaccactacacccagaagtccctgtctctgtccctgggcaaa

SEQ ID NO: 52 - DNA, 1185 bp, Artificial Sequence
  Description of Artificial Sequence Synthetic polynucleotide
atgacagttttggctccagcttggtcccctacaacctacctgctgctgctgttgctgctctcctctggcctgtctggcacccaggactgttccttccagcactcccctatctccagcgacttcgccgtgaagatcagagagctgtccgactatctgctgcaggactaccctgtgaccgtggccagcaatctgcaggacgaagaactgtgtggtggcctgtggcgactggtgttggctcagagatggatggaacggctgaaaaccgtggccggctctaagatgcagggcctgctggaaagagtgaacaccgagatccacttcgtgaccaagtgcgcctttcagcctcctccatcctgcctgagattcgtgcagaccaatatcgcccggctgctgcaagagacatccgagcagctggtggctctgaagccctggatcaccagacagaacttcgcccggtgtctggaactgcagtgtcagcctgacagctctaccctgcctccaccttggagccctagacctctggaagctaccgctccaaccgctcctcaagggggaccgtcagtcttcctcttccccccaaaacccaaggacaccctctacatcacccgggaacctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 53 - DNA, 1107 bp, Artificial Sequence
  Description of Artificial Sequence Synthetic polynucleotide
acccaggactgctccttccagcactcccctatctcttccgacttcgccgtgaagatcagagagctgtccgactacctgctgcaggactatcctgtgaccgtggccagcaacctgcaggatgaagaactgtgtggtggactgtggcgactggtgctggctcagagatggatggaaagactgaaaaccgtggccggctccaagatgcagggactgctggaaagagtgaacacagagatccacttcgtgaccaagtgcgcctttcagcctcctccatcctgcctgagattcgtgcagaccaatatcgcccggctgctgcaagagacatctgagcagctggtggccctgaagccttggatcaccagacagaacttcgcccggtgcctggaactgcagtgtcagcctgattcttctaccctgcctccaccttggagccctagacctttggaggctacagctcctactgctcctcaaggcggcccaagcgttttcctgtttcctccaaagcctaaggacaccctgtacatcacccgcgagcctgaagtgacatgcgtggtggtggatgtgtcccacgaggaccccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaacctcaggtttacaccctgccacctagccgggaagagatgaccaaaaaccaggtgtccctgacctgcctggtcaagggcttctacccatccgatatcgccgtggaatgggagtctaacggccagcctgagaacaactacaagaccacacctcctgtgctggactccgacggctcattcttcctgtactccaagctgacagtggacaagtctcggtggcagcagggcaacgtgttctcctgttctgtgatgcacgaggccctgcacaaccactacacccagaagtccctgtctctgtcccctggcaaa

SEQ ID NO: 54 - DNA, 1143 bp, Artificial Sequence
  Description of Artificial Sequence Synthetic polynucleotide
acacaggattgcagcttccagcacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccaagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctctggaagccacagctgagtctaagtacggccctccttgtcctccatgtcctgctccagaagctgctggcggcccttccgtgtttctgttccctccaaagcccaaggacaccctgtacatcacccgggaacccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgtccctgggcaaa

SEQ ID NO: 55 - DNA, 1143 bp, Artificial Sequence
  Description of Artificial Sequence Synthetic polynucleotide
acccaggactgctccttccagcactcccctatctcttccgacttcgccgtgaagatcagagagctgtccgactacctgctgcaggactatcctgtgaccgtggccagcaacctgcaggatgaagaactgtgtggtggactgtggcgactggtgctggctcagagatggatggaaagactgaaaaccgtggccggctccaagatgcagggactgctggaaagagtgaacacagagatccacttcgtgaccaagtgcgcctttcagcctcctccatcctgcctgagattcgtgcagaccaacatctcccggctgctgcaagagacatctgagcagctggtggccctgaagccttggatcacccggcagaacttctctcggtgcctggaactgcagtgtcagcctgattcttctaccctgcctccaccttggagccctagacctctggaagctaccgccgagtctaagtacggacctccttgtcctccatgtcctgctccagaagctgctggcggaccaagcgttttcctgtttcctccaaagcctaaggacaccctgtacatcacccgcgagcctgaagtgacatgcgtggtggtggatgtgtcccaagaggaccccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgcctagctccatcgaaaagaccatctccaaggccaagggccagccaagagaacctcaggtgtacacactgcctccaagccaagaggaaatgaccaagaaccaggtgtccctgacctgcctggtcaagggcttctacccatccgatatcgccgtggaatgggagtctaacggccagcctgagaacaactacaagaccacacctcctgtgctggactccgacggctccttctttctgtactctcgcctgaccgtggacaagtctagatggcaagagggcaacgtgttctcctgctctgtgatgcacgaggccctgcacaaccactacacccagaagtccctgtctctgtccctgggcaaa

SEQ ID NO: 56 - DNA, 1077 bp, Artificial Sequence
  Description of Artificial Sequence Synthetic polynucleotide
acacaggattgcagcttccagcacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccaagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctgggggaccgtcagtcttcctcttccccccaaaacccaaggacaccctctacatcacccgggaacctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 57 - DNA, 1077 bp, Artificial Sequence
  Description of Artificial Sequence Synthetic polynucleotide
acccaggactgctccttccagcactcccctatctcttccgacttcgccgtgaagatcagagagctgtccgactacctgctgcaggactatcctgtgaccgtggccagcaacctgcaggatgaagaactgtgtggtggactgtggcgactggtgctggctcagagatggatggaaagactgaaaaccgtggccggctccaagatgcagggactgctggaaagagtgaacacagagatccacttcgtgaccaagtgcgcctttcagcctcctccatcctgcctgagattcgtgcagaccaacatctcccggctgctgcaagagacatctgagcagctggtggccctgaagccttggatcacccggcagaacttctctcggtgcctggaactgcagtgtcagcctgattcttctaccctgcctccaccttggagtcctagacctggcggaccaagcgtgttcctgtttcctccaaagcctaaggacaccctgtacatcacccgcgagcctgaagtgacatgcgtggtggtggatgtgtcccacgaggaccccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaacctcaggtttacaccctgccacctagccgggaagagatgaccaaaaaccaggtgtccctgacctgcctggtcaagggcttctacccatccgatatcgccgtggaatgggagtctaacggccagcctgagaacaactacaagaccacacctcctgtgctggactccgacggctcattcttcctgtactccaagctgacagtggacaagtctcggtggcagcagggcaacgtgttctcctgttctgtgatgcacgaggccctgcacaaccactacacccagaagtccctgtctctgtcccctggcaaa

SEQ ID NO: 58 - DNA, 1128 bp, Artificial Sequence
  Description of Artificial Sequence Synthetic polynucleotide
acacaggattgcagcttccagcacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccaagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctgagtctaagtacggccctccttgtcctccatgtcctgctccagaagctgctggcggcccttccgtgtttctgttccctccaaagcctaaggacaccctgtacatcacccgggaacccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgtccctgggcaaa

SEQ ID NO: 59 - DNA, 1128 bp, Artificial Sequence
  Description of Artificial Sequence Synthetic polynucleotide
acccaggactgctccttccagcactcccctatctcttccgacttcgccgtgaagatcagagagctgtccgactacctgctgcaggactatcctgtgaccgtggccagcaacctgcaggatgaagaactgtgtggtggactgtggcgactggtgctggctcagagatggatggaaagactgaaaaccgtggccggctccaagatgcagggactgctggaaagagtgaacacagagatccacttcgtgaccaagtgcgcctttcagcctcctccatcctgcctgagattcgtgcagaccaacatctcccggctgctgcaagagacatctgagcagctggtggccctgaagccttggatcacccggcagaacttctctcggtgcctggaactgcagtgtcagcctgattcttctaccctgcctccaccttggagccctcggcctgaatctaagtatggccctccttgtcctccatgtcctgctccagaagctgctggcggaccaagcgttttcctgtttcctccaaagcctaaggacaccctgtacatcacccgcgagcctgaagtgacatgcgtggtggtggatgtgtcccaagaggaccccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgcctagctccatcgaaaagaccatctccaaggccaagggccagccaagagaacctcaggtgtacacactgcctccaagccaagaggaaatgaccaagaaccaggtgtccctgacctgcctggtcaagggcttctacccatccgatatcgccgtggaatgggagtctaacggccagcctgagaacaactacaagaccacacctcctgtgctggactccgacggctccttctttctgtactctcgcctgaccgtggacaagtctagatggcaagagggcaacgtgttctcctgctctgtgatgcacgaggccctgcacaaccactacacccagaagtccctgtctctgtccctgggcaaa

SEQ ID NO: 60 - DNA, 1128 bp, Artificial Sequence
  Description of Artificial Sequence Synthetic polynucleotide
acccaggactgttccttccagcactcccctatctccagcgacttcgccgtgaagatcagagagctgtccgactatctgctgcaggactaccctgtgaccgtggccagcaatctgcaggacgaagaactgtgtggtggcctgtggcgactggtgttggctcagagatggatggaacggctgaaaaccgtggccggctctaagatgcagggcctgctggaaagagtgaacaccgagatccacttcgtgaccaagtgcgcctttcagcctcctccatcctgcctgagattcgtgcagaccaatatcgcccggctgctgcaagagacatccgagcagctggtggctctgaagccctggatcaccagacagaacttcgcccggtgtctggaactgcagtgtcagcctgacagctctaccctgcctccaccttggagccctagacctgagtctaagtacggccctccttgtcctccatgtcctgctccagaagctgctggcggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggccagcctagggaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggcaaa

SEQ ID NO: 61 - DNA, 1128 bp, Artificial Sequence
  Description of Artificial Sequence Synthetic polynucleotide
acccaggactgttccttccagcactcccctatctccagcgacttcgccgtgaagatcagagagctgtccgactatctgctgcaggactaccctgtgaccgtggccagcaatctgcaggacgaagaactgtgtggtggcctgtggcgactggtgttggctcagagatggatggaacggctgaaaaccgtggccggctctaagatgcagggcctgctggaaagagtgaacaccgagatccacttcgtgaccaagtgcgcctttcagcctcctccatcctgcctgagattcgtgcagaccaatatcgcccggctgctgcaagagacatccgagcagctggtggctctgaagccctggatcaccagacagaacttcgcccggtgtctggaactgcagtgtcagcctgacagctctaccctgcctccaccttggagccctagacctgagtctaagtacggccctccttgtcctccatgtcctgctccagaagctgctggcggcccttccgtgtttctgttccctccaaagcccaaggacaccctgtacatcacccgggaacccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgtccctgggcaaa

SEQ ID NO: 62 - DNA, 1167 bp, Artificial Sequence
  Description of Artificial Sequence Synthetic polynucleotide
acccctgactgctacttcagccactctcctatctccagcaacttcaaagtgaagttccgcgagctgaccgaccatctgctgaaggactatcctgtgaccgtggccgtgaacctgcaggacgaaaagcactgcaaggccctgtggtccctgtttctggcccagagatggatcgagcagctgaaaaccgtggctggctccaagatgcagaccctgctggaagatgtgaacaccgagatccacttcgtgaccagctgcaccttccagcctctgcctgagtgcctgagattcgtgcagaccaacatctcccacctgttgaaggacacatgcacccagctgctggccctgaagccttgtatcggcaaggcctgccagaacttctcccggtgtctggaagtgcagtgccagcctgactcctccacactgctgccacctagaagccctatcgctctggaagctaccgagctgcctgagcctagaggccctaccatcaagccttgtcctccatgcaagtgccccgctcctaatgctgctggtggcccttccgtgttcatcttcccacctaagatcaaggacgtgctgatgatctccctgtctcctatcgtgacctgcgtggtggtggacgtgtccgaggatgatcctgacgtgcagatcagttggttcgtgaacaacgtggaagtgcacaccgctcagacccagacacacagagaggactacaacagcaccctgagagtggtgtctgccctgcctatccagcaccaggattggatgtccggcaaagaattcaagtgcaaagtgaacaacaaggacctgggcgctcccatcgagcggaccatctctaagcctaagggatccgtcagagcccctcaggtgtacgttctgcctccacctgaggaagagatgaccaagaaacaagtgaccctgacctgcatggtcaccgacttcatgcccgaggacatctacgtggaatggaccaacaacggcaagaccgagctgaactacaagaacaccgagcctgtgctggactccgacggctcctacttcatgtactccaagctgcgcgtcgagaagaagaactgggtcgagagaaactcctactcctgctccgtggtgcacgagggcctgcacaatcaccacaccaccaagtccttctctcggacccctggcaaa

SEQ ID NO: 63 - DNA, 1167 bp, Artificial Sequence
  Description of Artificial Sequence Synthetic polynucleotide
acccctgactgctacttcagccactctcctatctccagcaacttcaaagtgaagttccgcgagctgaccgaccatctgctgaaggactatcctgtgaccgtggccgtgaacctgcaggacgaaaagcactgcaaggccctgtggtccctgtttctggcccagagatggatcgagcagctgaaaaccgtggctggctccaagatgcagaccctgctggaagatgtgaacaccgagatccacttcgtgaccagctgcaccttccagcctctgcctgagtgcctgagattcgtgcagaccaacatctcccacctgttgaaggacacatccacccagctgctggccctgaagccttgtatcggcaaggcctgccagaacttctcccggtgtctggaagtgcagtgccagcctgactcctccacactgctgccacctagaagccctatcgctctggaagctaccgagctgcctgagcctagaggccctaccatcaagccttgtcctccatgcaagtgccccgctcctaatgctgctggtggcccttccgtgttcatcttcccacctaagatcaaggacgtgctgatgatctccctgtctcctatcgtgacctgcgtggtggtggacgtgtccgaggatgatcctgacgtgcagatcagttggttcgtgaacaacgtggaagtgcacaccgctcagacccagacacacagagaggactacaacagcaccctgagagtggtgtctgccctgcctatccagcaccaggattggatgtccggcaaagaattcaagtgcaaagtgaacaacaaggacctgggcgctcccatcgagcggaccatctctaagcctaagggatccgtcagagcccctcaggtgtacgttctgcctccacctgaggaagagatgaccaagaaacaagtgaccctgacctgcatggtcaccgacttcatgcccgaggacatctacgtggaatggaccaacaacggcaagaccgagctgaactacaagaacaccgagcctgtgctggactccgacggctcctacttcatgtactccaagctgcgcgtcgagaagaagaactgggtcgagagaaactcctactcctgctccgtggtgcacgagggcctgcacaatcaccacaccaccaagtccttctctcggacccctggcaaa

SEQ ID NO: 64 - DNA, 1107 bp, Artificial Sequence
  Description of Artificial Sequence Synthetic polynucleotide
acacaggattgcagcttccagcacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccaagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctctggaagccacagctcccaccgctcctcaaggcggaccgtcagtctttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtgtacacaaagcctccaagccgggaagagatgaccaagaaccaggtgtccctgagctgcctggtcaagggcttttaccccagcgacattgccgtggaatgggagagcaatggccagcctgagaacaactacaagaccaccgtgcctgtgctggacagcgacggctcttttagactggccagctacctgaccgtggacaagagcagatggcagcagggcaacgtgttcagctgcagcgtgatgcacgaggccctgcacaaccactacacccagaagtccctgtctctgagccccggcaaa

SEQ ID NO: 65 - DNA, 1107 bp, Artificial Sequence
  Description of Artificial Sequence Synthetic polynucleotide
acacaggattgcagcttccagtacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccaagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctctggaagccacagctcccaccgctcctcaaggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 66 - DNA, 1107 bp, Artificial Sequence
  Description of Artificial Sequence Synthetic polynucleotide
acacaggattgcagcttccagcacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccgagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctctggaagccacagctcccaccgctcctcaaggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 67 - DNA, 1107 bp, Artificial Sequence
  Description of Artificial Sequence Synthetic polynucleotide
acacaggattgcagcttccagtacagccccatcagcagcgatttcgccgtgaagatcagagagctgagcgactacctgctgcaggactaccctgtgaccgtggccagcaatctgcaggacgaagaactgtgtggtggactgtggcgactggtgctggctcagagatggatggaacggctgaaaacagtggccggcagcaagatgcagggactgctggaaagagtgaacaccgagatccacttcgtgaccgagtgcgccttccagcctcctccatcctgcctgagattcgtgcagaccaacatcagcagactgctgcaagagacaagcgagcagctggtggccctgaagccttggatcaccagacagaacttcagccggtgcctggaactgcagtgtcagcccgatagcagcacactgcctccgccttggagtcctagacctctggaagccacagctcccaccgctcctcaaggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 68 - DNA, 1143 bp, Artificial Sequence
  Description of Artificial Sequence Synthetic polynucleotide
acccaggactgttccttccagcactcccctatctccagcgacttcgccgtgaagatcagagagctgtccgactatctgctgcaggactaccctgtgaccgtggccagcaatctgcaggacgaagaactgtgtggtggcctgtggcgactggtgttggctcagagatggatggaacggctgaaaaccgtggccggctctaagatgcagggcctgctggaaagagtgaacaccgagatccacttcgtgaccaagtgcgcctttcagcctcctccatcctgcctgagattcgtgcagaccaatatcgcccggctgctgcaagagacatccgagcagctggtggctctgaagccctggatcaccagacagaacttcgcccggtgtctggaactgcagtgtcagcctgacagctctaccctgcctccaccttggagccctagacctctggaagctaccgctgagtctaagtacggccctccttgtcctccatgtcctgctccagaagctgctggcggcccttccgtgtttctgttccctccaaagcccaaggacaccctgtacatcacccgggaacccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgtccctgggcaaa

SEQ ID NO: 69 - DNA, 1143 bp, Artificial Sequence
  Description of Artificial Sequence Synthetic polynucleotide
acccaggactgttccttccagcactcccctatctccagcgacttcgccgtgaagatcagagagctgtccgactatctgctgcaggactaccctgtgaccgtggccagcaatctgcaggacgaagaactgtgtggtggcctgtggcgactggtgttggctcagagatggatggaacggctgaaaaccgtggccggctctaagatgcagggcctgctggaaagagtgaacaccgagatccacttcgtgaccaagtgcgcctttcagcctcctccatcctgcctgagattcgtgcagaccaacatctcccggctgctgcaagagacatccgagcagctggtggctctgaagccctggatcacccggcagaacttctctcggtgtctggaactgcagtgtcagcctgatgctgccgctttgcctccaccttggagccctagacctctggaagctaccgccgagtctaagtacggacctccttgtcctccatgtcctgctccagaagctgctggcggcccttccgtgtttctgttccctccaaagcccaaggacaccctgtacatcacccgggaacccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgtccctgggcaaa

SEQ ID NO: 70 - DNA, 1143 bp, Artificial Sequence
  Description of Artificial Sequence Synthetic polynucleotide
acccaggactgttccttccagcactcccctatctccagcgacttcgccgtgaagatcagagagctgtccgactatctgctgcaggactaccctgtgaccgtggccagcaatctgcaggacgaagaactgtgtggtggcctgtggcgactggtgttggctcagagatggatggaacggctgaaaaccgtggccggctctaagatgcagggcctgctggaaagagtgaacaccgagatccacttcgtgaccaagtgcgcctttcagcctcctccatcctgcctgagattcgtgcagaccaacatctcccggctgctgcaagagacatccgagcagctggtggctctgaagccctggatcacccggcagaacttctctcggtgtctggaactgcagtgtcagcctgatgctgccgctttgcctcctccttgggctcctcgacctctggaagctacagccgaggctaagtatggccctccttgtcctccatgtcctgctccagaagctgctggcggcccttccgtgtttctgttccctccaaagcccaaggacaccctgtacatcacccgggaacccgaagtgacctgcgtggtggtggatgtgtcccaggaagatcccgaggtgcagttcaattggtacgtggacggcgtggaagtgcacaacgccaagaccaagcccagagaggaacagttcaacagcacctaccgggtggtgtccgtgctgacagtgctgcaccaggactggctgaacggcaaagagtacaagtgcaaggtgtccaacaagggcctgcccagctccatcgagaaaaccatcagcaaggccaagggccagccccgcgaaccccaggtgtacacactgcctccaagccaggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtacagcagactgaccgtggacaagagccggtggcaggaaggcaacgtgttcagctgcagcgtgatgcacgaggccctgcacaaccactacacccagaagtccctgtctctgtccctgggcaaa

SEQ ID NO: 71 - protein, 147 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRP

SEQ ID NO: 72 - protein, 148 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPL

SEQ ID NO: 73 - protein, 149 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLE

SEQ ID NO: 74 - protein, 150 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEA

SEQ ID NO: 75 - protein, 151 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEAT

SEQ ID NO: 76 - protein, 152 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATA

SEQ ID NO: 77 - protein, 153 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAP

SEQ ID NO: 78 - protein, 154 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPT

SEQ ID NO: 79 - protein, 155 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTA

SEQ ID NO: 80 - protein, 156 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAP

SEQ ID NO: 81 - protein, 157 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQ

SEQ ID NO: 82 - protein, 17 aa, Unknown
  Description of Unknown Serum albumin signal peptide
KWVTFISLLFLFSSAYS

SEQ ID NO: 83 - protein, 26 aa, Unknown
  Description of Unknown FLT3L signal peptide
MTVLAPAWSPTTYLLLLLLLSSGLSG

SEQ ID NO: 84 - protein, 27 aa, Unknown
  Description of Unknown FLT3L signal peptide
MTVLAPAWSPNSSLLLLLLLLSPCLRG

SEQ ID NO: 85 - protein, 5 aa, Homo sapiens
PTAPQ

SEQ ID NO: 86 - protein, 6 aa, Homo sapiens
APTAPQ

SEQ ID NO: 87 - protein, 7 aa, Homo sapiens
TAPTAPQ

SEQ ID NO: 88 - protein, 8 aa, Homo sapiens
ATAPTAPQ

SEQ ID NO: 89 - protein, 9 aa, Homo sapiens
EATAPTAPQ

SEQ ID NO: 90 - protein, 10 aa, Homo sapiens
LEATAPTAPQ

SEQ ID NO: 91 - protein, 7 aa, Homo sapiens
PTAPQPP

SEQ ID NO: 92 - protein, 8 aa, Homo sapiens
APTAPQPP

SEQ ID NO: 93 - protein, 9 aa, Homo sapiens
TAPTAPQPP

SEQ ID NO: 94 - protein, 10 aa, Homo sapiens
ATAPTAPQPP

SEQ ID NO: 95 - protein, 11 aa, Homo sapiens
EATAPTAPQPP

SEQ ID NO: 96 - protein, 12 aa, Homo sapiens
LEATAPTAPQPP

SEQ ID NO: 97 - protein, 5 aa, Homo sapiens
ESKYG

SEQ ID NO: 98 - protein, 159 aa, Mus musculus
TPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPR

SEQ ID NO: 99 - protein, 20 aa, Artificial Sequence
  Description of Artificial Sequence Synthetic peptide
  MISC_FEATURE (1)..(20): This sequence may encompass 3-4 “Gly Gly Gly Gly Ser” repeating units
GGGGSGGGGSGGGGSGGGGS

SEQ ID NO: 100 - protein, 12 aa, Artificial Sequence
  Description of Artificial Sequence Synthetic peptide
ESKYGPPCPPCP

SEQ ID NO: 101 - protein, 15 aa, Artificial Sequence
  Description of Artificial Sequence Synthetic peptide
EPKSCDKTHTCPPCP

SEQ ID NO: 102 - protein, 232 aa, Artificial Sequence
  Description of Artificial Sequence Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 212 aa, Artificial Sequence
  Description of Artificial Sequence Synthetic polypeptide
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 212 aa, Artificial Sequence
  Description of Artificial Sequence Synthetic polypeptide
GGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 229 aa, Artificial Sequence
  Description of Artificial Sequence Synthetic polypeptide
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 106 - protein, 229 aa, Artificial Sequence
  Description of Artificial Sequence Synthetic polypeptide
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 107 - protein, 229 aa, Artificial Sequence
  Description of Artificial Sequence Synthetic polypeptide
ESKYGPPCPPCPAPEFEGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 108 - protein, 15 aa, Artificial Sequence
  Description of Artificial Sequence Synthetic peptide
RRCPLYISYDPVCRR

SEQ ID NO: 109 - protein, 19 aa, Artificial Sequence
  Description of Artificial Sequence Synthetic peptide
RRRRCPLYISYDPVCRRRR

SEQ ID NO: 110 - protein, 20 aa, Artificial Sequence
  Description of Artificial Sequence Synthetic peptide
EPKSCDKTHTCPPCPAPELL

SEQ ID NO: 111 - protein, 226 aa, Artificial Sequence
  source: /note=“Description of Artificial Sequence Synthetic polypeptide”
DKTHTCPPCPAPPVAGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 141 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPP

SEQ ID NO: 113 - protein, 235 aa, Homo sapiens
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 114 - protein, 367 aa, Artificial Sequence
  source: /note=“Description of Artificial Sequence Synthetic polypeptide”
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPDKTHTCPPCPAPPVAGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 393 aa, Artificial Sequence
  source: /note=“Description of Artificial Sequence Synthetic polypeptide”
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPDKTHTCPPCPAPPVAGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 5 aa, Artificial Sequence
  source: /note=“Description of Artificial Sequence Synthetic peptide”
PVAGP

SEQ ID NO: 117 - protein, 235 aa, Artificial Sequence; identical to SEQ ID NO: 113
  source: /note=“Description of Artificial Sequence Synthetic peptide”